SEQUENCE LISTING (453 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 609 aa, Homo sapiens [sequence omitted]

SEQ ID NO: 3 - protein, 8 aa, unidentified
  source 1..8: note = MMP7 cleavage domain sequence
KRALGLPG

SEQ ID NO: 4 - protein, 40 aa, unidentified
  source 1..40: note = MMP7 cleavage domain sequence
DEDEDEDEDEDEDEDERPLALWRSDRDRDRDRDRDRDRDR

SEQ ID NO: 5 - skipped

SEQ ID NO: 6 - protein, 5 aa, unidentified
  source 1..5: note = MMP9 cleavage domain sequence
LEATA

SEQ ID NO: 7 - protein, 10 aa, unidentified
  source 1..10: note = MMP11 cleavage domain sequence
GGAANLVRGG

SEQ ID NO: 8 - protein, 10 aa, unidentified
  source 1..10: note = MMP14 cleavage domain sequence
SGRIGFLRTA

SEQ ID NO: 9 - protein, 6 aa, unidentified
  source 1..6: note = MMP cleavage domain sequence
PLGLAG

SEQ ID NO: 10 - protein, 6 aa, unidentified
  VARIANT 6: note = Any amino acid
  source 1..6: note = MMP cleavage domain sequence
PLGLAX

SEQ ID NO: 11 - protein, 6 aa, unidentified
  MOD_RES 4: note = s-methyl cysteine
  source 1..6: note = MMP cleavage domain sequence
PLGXAG

SEQ ID NO: 12 - protein, 8 aa, unidentified
  source 1..8: note = MMP cleavage domain sequence
ESPAYYTA

SEQ ID NO: 13 - protein, 6 aa, unidentified
  source 1..6: note = MMP cleavage domain sequence
RLQLKL

SEQ ID NO: 14 - protein, 7 aa, unidentified
  source 1..7: note = MMP cleavage domain sequence
RLQLKAC

SEQ ID NO: 15 - protein, 7 aa, unidentified
  MOD_RES 3: note = citrulline
  MOD_RES 5: note = homophenylalanine
  source 1..7: note = MMP2, MMP9, MMP14 cleavage domain sequence
EPXGXYL

SEQ ID NO: 16 - protein, 5 aa, unidentified
  source 1..5: note = Urokinase plasminogen activator (uPA) cleavage domain sequence
SGRSA

SEQ ID NO: 17 - protein, 4 aa, unidentified
  source 1..4: note = Urokinase plasminogen activator (uPA) cleavage domain sequence
DAFK

SEQ ID NO: 18 - protein, 5 aa, unidentified
  source 1..5: note = Urokinase plasminogen activator (uPA) cleavage domain sequence
GGGRR

SEQ ID NO: 19 - protein, 4 aa, unidentified
  source 1..4: note = Lysosomal Enzyme cleavage domain sequence
GFLG

SEQ ID NO: 20 - protein, 4 aa, unidentified
  source 1..4: note = Lysosomal Enzyme cleavage domain sequence
ALAL

SEQ ID NO: 21-22 - skipped (2 entries)

SEQ ID NO: 23 - protein, 5 aa, unidentified
  MOD_RES 3: note = s-ethyl cysteine
  source 1..5: note = Cathepsin D cleavage domain sequence
PIXFF

SEQ ID NO: 24 - protein, 8 aa, unidentified
  source 1..8: note = Cathepsin K cleavage domain sequence
GGPRGLPG

SEQ ID NO: 25 - protein, 6 aa, unidentified
  source 1..6: note = Prostate Specific Antigen cleavage domain sequence
HSSKLQ

SEQ ID NO: 26 - protein, 7 aa, unidentified
  source 1..7: note = Prostate Specific Antigen cleavage domain sequence
HSSKLQL

SEQ ID NO: 27 - protein, 9 aa, unidentified
  source 1..9: note = Prostate Specific Antigen cleavage domain sequence
HSSKLQEDA

SEQ ID NO: 28 - protein, 10 aa, unidentified
  source 1..10: note = Herpes Simplex Virus Protease cleavage domain sequence
LVLASSSFGY

SEQ ID NO: 29 - protein, 10 aa, unidentified
  source 1..10: note = HIV Protease cleavage domain sequence
GVSQNYPIVG

SEQ ID NO: 30 - protein, 10 aa, unidentified
  source 1..10: note = CMV Protease cleavage domain sequence
GVVQASCRLA

SEQ ID NO: 31 - skipped

SEQ ID NO: 32 - protein, 6 aa, unidentified
  source 1..6: note = Thrombin cleavage domain sequence
DPRSFL

SEQ ID NO: 33 - protein, 6 aa, unidentified
  source 1..6: note = Thrombin cleavage domain sequence
PPRSFL

SEQ ID NO: 34 - protein, 4 aa, unidentified
  source 1..4: note = Caspase-3 cleavage domain sequence
DEVD

SEQ ID NO: 35 - protein, 5 aa, unidentified
  source 1..5: note = Caspase-3 cleavage domain sequence
DEVDP

SEQ ID NO: 36 - protein, 8 aa, unidentified
  source 1..8: note = Caspase-3 cleavage domain sequence
KGSGDVEG

SEQ ID NO: 37 - protein, 6 aa, unidentified
  source 1..6: note = Interleukin 1-beta converting enzyme cleavage domain sequence
GWEHDG

SEQ ID NO: 38 - protein, 7 aa, unidentified
  source 1..7: note = Enterokinase cleavage domain sequence
EDDDDKA

SEQ ID NO: 39 - protein, 9 aa, unidentified
  source 1..9: note = FAP cleavage domain sequence
KQEQNPGST

SEQ ID NO: 40 - protein, 6 aa, unidentified
  source 1..6: note = Kallikrein 2 cleavage domain sequence
GKAFRR

SEQ ID NO: 41 - protein, 4 aa, unidentified
  source 1..4: note = Plasmin cleavage domain sequence
DAFK

SEQ ID NO: 42 - protein, 4 aa, unidentified
  source 1..4: note = Plasmin cleavage domain sequence
DVLK

SEQ ID NO: 43 - protein, 4 aa, unidentified
  source 1..4: note = Plasmin cleavage domain sequence
DAFK

SEQ ID NO: 44 - protein, 7 aa, unidentified
  source 1..7: note = TOP cleavage domain sequence
ALLLALL

SEQ ID NO: 45 - protein, 652 aa, synthetic construct
QVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 46 - protein, 652 aa, synthetic construct
QVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 47 - protein, 553 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 48 - protein, 553 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 49 - protein, 553 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 50 - protein, 682 aa, synthetic construct
QVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 51 - protein, 667 aa, synthetic construct
QVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGMKGLPGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 52 - protein, 682 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSHHHHHH

SEQ ID NO: 53 - protein, 393 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 54 - protein, 423 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 55 - protein, 682 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSHHHHHH

SEQ ID NO: 56 - protein, 682 aa, synthetic construct
QVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 57 - protein, 393 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 58 - protein, 423 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 59 - protein, 669 aa, synthetic construct
QVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSGGGGSGGGGSGGGGSDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKHHHHHHEPEA

SEQ ID NO: 60 - protein, 669 aa, synthetic construct
QVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKGGGGSGGGGSGGGGSQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSHHHHHHEPEA

SEQ ID NO: 61 - protein, 689 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSGGGGSGGGGSGGGGSDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSHHHHHHEPEA

SEQ ID NO: 62 - protein, 689 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKGGGGSGGGGSGGGGSQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSHHHHHHEPEA

SEQ ID NO: 63 - protein, 272 aa, unidentified
  source 1..272: note = IL2Ra sequence
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 64 - protein, 551 aa, unidentified
  source 1..551: note = IL2Rb sequence
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 65 - protein, 369 aa, unidentified
  source 1..369: note = IL2Rg sequence
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 66 - protein, 520 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHH

SEQ ID NO: 67 - protein, 524 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASHHHHHH

SEQ ID NO: 68 - protein, 940 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSGGGGSGGGGSGGGGSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 69 - protein, 1069 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSGGGGSGGGGSGGGGSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSHHHHHHEPEA

SEQ ID NO: 70 - protein, 1069 aa, synthetic construct
QVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSGGGGSGGGGSGGGGSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 71 - protein, 940 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSGGGGSGGGGSGGGGSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHHEPEA

SEQ ID NO: 72 - protein, 940 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHHEPEA

SEQ ID NO: 73 - protein, 940 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 74 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 75 - protein, 215 aa, Mus musculus
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 76 - protein, 862 aa, unidentified
  source 1..862: note = IL12Rb-2 sequence
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 77 - protein, 662 aa, unidentified
  source 1..662: note = IL12Rb-1 sequence
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 78 - protein, 328 aa, Homo sapiens; identical to SEQ ID NO: 74

SEQ ID NO: 79 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 80 - protein, 399 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 81 - protein, 547 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 82 - protein, 547 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCGGGGSGGGGSGGGGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 83 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 84 - protein, 155 aa, Mus musculus
MNATHCILALQLFLMAVSGCYCHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 85 - protein, 699 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 86 - protein, 436 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 87 - protein, 428 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 88 - protein, 489 aa, unidentified
  source 1..489: note = IFNgR1 sequence
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 89 - protein, 337 aa, unidentified
  source 1..337: note = IFNgR2 sequence
MRPTLLWSLLLLLGVFAAAAAAPPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 90 - protein, 528 aa, synthetic construct
QVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 91 - protein, 658 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSHHHHHH

SEQ ID NO: 92 - protein, 1337 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHH

SEQ ID NO: 93 - protein, 1485 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCGGGGSGGGGSGGGGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHH

SEQ ID NO: 94 - protein, 698 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCGGGGSGGGGSGGGGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 95 - protein, 699 aa, synthetic construct; identical to SEQ ID NO: 85

SEQ ID NO: 96 - protein, 554 aa, synthetic construct
MDMRVPAQLLGLLLLWLRGARCQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 97 - protein, 554 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSHHHHHHEPEA

SEQ ID NO: 98 - protein, 702 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSHHHHHHEPEA

SEQ ID NO: 99 - protein, 702 aa, synthetic construct
MDMRVPAQLLGLLLLWLRGARCQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 100 - protein, 702 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSHHHHHHEPEA

SEQ ID NO: 101 - protein, 832 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARGVGAFRPYRKHEWGQGTLVTVSRGGGGSGGGGSGGGGSSSELTQDPAVSVALGQTVRITCQGDSLRSYYASWYQQKPGQAPVLVIYGKNNRPSGIPDRFSGSSSGNTASLTTTGAQAEDEADYYCNSSPFEHNLVVFGGGTKLTVLHHHHHHEPEA

SEQ ID NO: 102 - protein, 276 aa, synthetic construct
MDMRVPAQLLGLLLLWLRGARCEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARGVGAFRPYRKHEWGQGTLVTVSRGGGGSGGGGSGGGGSSSELTQDPAVSVALGQTVRITCQGDSLRSYYASWYQQKPGQAPVLVIYGKNNRPSGIPDRFSGSSSGNTASLTTTGAQAEDEADYYCNSSPFEHNLVVFGGGTKLTVLHHHHHHEPEA

SEQ ID NO: 103 - protein, 573 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCHHHHHHEPEA

SEQ ID NO: 104 - protein, 573 aa, synthetic construct
MDMRVPAQLLGLLLLWLRGARCHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 105 - protein, 1511 aa, synthetic construct
MDMRVPAQLLGLLLLWLRGARCHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHHEPEA

SEQ ID NO: 106 - protein, 1511 aa, synthetic construct
MDMRVPAQLLGLLLLWLRGARCEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCHHHHHHEPEA

SEQ ID NO: 107 - protein, 2110 aa, synthetic construct
MDMRVPAQLLGLLLLWLRGARCEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHHEPEA

SEQ ID NO: 108 - protein, 2120 aa, synthetic construct
MDMRVPAQLLGLLLLWLRGARCEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSGGGGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGGSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHHEPEA

SEQ ID NO: 109 - protein, 2130 aa, synthetic construct
MDMRVPAQLLGLLLLWLRGARCEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSGGGGSGGGGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGGSGGGGSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHHEPEA

SEQ ID NO: 110 - protein, 703 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 111 - protein, 832 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSHHHHHHEPEA

SEQ ID NO: 112 - protein, 832 aa, synthetic construct
MDMRVPAQLLGLLLLWLRGARCQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 113 - protein, 549 aa, synthetic construct
MDMRVPAQLLGLLLLWLRGARCRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSHHHHHH

SEQ ID NO: 114 - protein, 579 aa, synthetic construct
MDMRVPAQLLGLLLLWLRGARCRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSSGGPGPAGMKGLPGSGGGGSGGGGSGGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSHHHHHH

SEQ ID NO: 115 - protein, 809 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 116 - protein, 938 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 117 - protein, 938 aa, synthetic construct; identical to SEQ ID NO: 116

SEQ ID NO: 118 - protein, 938 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSHHHHHH

SEQ ID NO: 119 - protein, 962 aa, synthetic construct
MDMRVPAQLLGLLLLWLRGARCIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 120 - protein, 832 aa, synthetic construct
MDMRVPAQLLGLLLLWLRGARCIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHHEPEA

SEQ ID NO: 121 - protein, 1091 aa, synthetic construct
MDMRVPAQLLGLLLLWLRGARCIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSHHHHHHEPEA

SEQ ID NO: 122 - protein, 1091 aa, synthetic construct
MDMRVPAQLLGLLLLWLRGARCQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 123 - protein, 1091 aa, synthetic construct
MDMRVPAQLLGLLLLWLRGARCQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 124 - protein, 674 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSHHHHHH

SEQ ID NO: 125 - protein, 674 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 126 - protein, 425 aa, synthetic construct
MDMRVPAQLLGLLLLWLRGARCELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 127 - protein, 425 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQHHHHHH

SEQ ID NO: 128 - protein, 555 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 129 - protein, 555 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 130 - protein, 555 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 131 - protein, 575 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 132 - protein, 704 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGMKGLPGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSHHHHHH

SEQ ID NO: 133 - protein, 689 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 134 - protein, 545 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 135 - protein, 575 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 136 - protein, 421 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 137 - protein, 806 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGSGSGSGSGSGSGSGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGSGSGSGSGSGSGSGSQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGSGSGSGSGSGSGSGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 138 - protein, 676 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGSGSGSGSGSGSGSGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGSGSGSGSGSGSGSGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 139 - protein, 421 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGSGSGSGSGSGSGSGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 140 - protein, 420 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 141 - protein, 550 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 142 - protein, 420 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 143 - protein, 290 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 144 - protein, 291 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 145 - protein, 689 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 146 - protein, 704 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSHHHHHH

SEQ ID NO: 147 - protein, 674 aa, synthetic construct
MDMRVPAQLLGLLLLWLRGARCQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 148 - protein, 704 aa, synthetic construct
MDMRVPAQLLGLLLLWLRGARCEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSHHHHHH

SEQ ID NO: 149 - protein, 704 aa, synthetic construct
MDMRVPAQLLGLLLLWLRGARCQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 150 - protein, 689 aa, synthetic construct
MDMRVPAQLLGLLLLWLRGARCEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSHHHHHH

SEQ ID NO: 151 - protein, 700 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSRGETGPAAPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 152 - protein, 276 aa, synthetic construct; identical to SEQ ID NO: 102

SEQ ID NO: 153 - protein, 279 aa, synthetic construct
MDMRVPAQLLGLLLLWLRGARCQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSGGGGSGGGGSGGGGSDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKHHHHHHEPEA

SEQ ID NO: 154 - protein, 279 aa, synthetic construct
MDMRVPAQLLGLLLLWLRGARCQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSSGGPGPAGMKGLPGSDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKHHHHHHEPEA

SEQ ID NO: 155 - protein, 279 aa, synthetic construct
MDMRVPAQLLGLLLLWLRGARCDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKGGGGSGGGGSGGGGSQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSHHHHHHEPEA

SEQ ID NO: 156 - protein, 279 aa, synthetic construct
MDMRVPAQLLGLLLLWLRGARCDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKSGGPGPAGMKGLPGSQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSHHHHHHEPEA

SEQ ID NO: 157 - protein, 295 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 158 - protein, 765 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHHEPEA

SEQ ID NO: 159 - protein, 765 aa, synthetic construct; identical to SEQ ID NO: 158

SEQ ID NO: 160 - protein, 765 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHHEPEA

SEQ ID NO: 161 - protein, 708 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSHHHHHHEPEA

SEQ ID NO: 162 - protein, 708 aa, synthetic construct
MDMRVPAQLLGLLLLWLRGARCQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 163 - protein, 708 aa, synthetic construct
MDMRVPAQLLGLLLLWLRGARCQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 164 - protein, 161 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 165 - protein, 708 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKGGGGSGGGGSGGGGSQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSHHHHHHEPEA

SEQ ID NO: 166 - protein, 708 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 167 - protein, 708 aa, synthetic construct
MDMRVPAQLLGLLLLWLRGARCQVQLQESGGGLVQAGGSLRLSCAASGRIFSIDIMSWYRQAPGKQRELVARITRGGTISYDDSVKGRFTISRDNAKNTVYLQMNSLKPEDTGVYYCNALYGTDYWGKGTQVTVSSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 168 - protein, 450 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 169 - protein, 198 aa, synthetic construct
MDMRVPAQLLGLLLLWLRGARCCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKHHHHHHEPEA

SEQ ID NO: 170 - protein, 328 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKHHHHHHEPEA

SEQ ID NO: 171 - protein, 328 aa, synthetic construct
MDMRVPAQLLGLLLLWLRGARCCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 172 - protein, 1396 aa, synthetic construct
MDMRVPAQLLGLLLLWLRGARCEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHHEPEA

SEQ ID NO: 173 - protein, 797 aa, synthetic construct
MDMRVPAQLLGLLLLWLRGARCEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKHHHHHHEPEA

SEQ ID NO: 174 - protein, 797 aa, synthetic construct
MDMRVPAQLLGLLLLWLRGARCCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHHEPEA

SEQ ID NO: 175 - protein, 587 aa, synthetic construct
MDMRVPAQLLGLLLLWLRGARCQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 176 - protein, 587 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSQVQLQESGGGLAQAGGSLSLSCAASGFTVSNSVMAWYRQTPGKQREFVAIINSVGSTNYADSVKGRFTISRDNAKNTVYLQMNNLKPEDTAVYVCNRNFDRIYWGQGTQVTVSSHHHHHHEPEA

SEQ ID NO: 177 - protein, 458 aa, synthetic construct
MDMRVPAQLLGLLLLWLRGARCEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHHEPEA

SEQ ID NO: 178 - protein, 579 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 179 - protein, 579 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPPGGPAGIGPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPPGGPAGIGPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 180 - protein, 579 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 181 - protein, 581 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPPLAQKLKSSPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPPLAQKLKSSPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 182 - protein, 613 aa, synthetic construct
MDMRVPAQLLGLLLLWLRGARCAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPPGGPAGIGALFKSSFPPLAQKLKSSPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPPGGPAGIGALFKSSFPPLAQKLKSSPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 183-190 - skipped (8 entries)

SEQ ID NO: 191 - protein, 267 aa, synthetic construct
MDMRVPAQLLGLLLLWLRGARCEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 192 - protein, 269 aa, synthetic construct
MDMRVPAQLLGLLLLWLRGARCQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 193 - protein, 171 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEHHHHHH

SEQ ID NO: 194 - protein, 520 aa, synthetic construct; identical to SEQ ID NO: 66

SEQ ID NO: 195 - protein, 141 aa, synthetic construct
QVQLQESGGGLVQAGGSLRLSCAASGRTFSSVYDMGWFRQAPGKDREFVARITESARNTRYADSVRGRFTISRDNAKNTVYLQMNNLELEDAAVYYCAADPQTVVVGTPDYWGQGTQVTVSSAAAYPYDVPDYGSHHHHHH

SEQ ID NO: 196 - protein, 247 aa, synthetic construct
QSVLTQPPSVSGAPGQRVTISCTGSSSNIGSNTVKWYQQLPGTAPKLLIYGNDQRPSGVPDRFSGSKSGTSASLAITGLQAEDEADYYCQSYDRYTHPAYVFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 197 - protein, 247 aa, synthetic construct
QSVLTQPPSVSGAPGQRVTISCTGSSSNIGSNTVKWYQQLPGTAPKLLIYYNDQRPSGVPDRFSGSKSGTSASLAITGLQAEDEADYYCQSYDRYTHPAYVFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 198 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARHGSHDNWGQGTMVTVSSHHHHHH

SEQ ID NO: 199 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYEGSNKYYADSVKGRFTISRDNSKNTLYLQMNSLRAEDTAVYYCKTHGSHDNWGQGTMVTVSSHHHHHH

SEQ ID NO: 200 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYAESVKGRFTISRDNSKNTLYLQMNSLRAEDTAVYYCKTHGSHDNWGQGTMVTVSSHHHHHH

SEQ ID NO: 201 - protein, 247 aa, synthetic construct
QSVLTQPPSVSGAPGQRVTISCSGSRSNIGSQTVKWYQQLPGTAPKLLIYYNDQRPSGVPDRFSGSKSGTSASLAITGLQAEDEADYYCQSYERYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 202 - protein, 247 aa, synthetic construct
QSVLTQPPSVSGAPGQRVTISCSGSRSNIGSQTVKWYQQLPGTAPKLLIYYNDQRPSGVPDRFSGSKSGTSASLAITGLQAEDEADYYCQSYSRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 203 - protein, 247 aa, synthetic construct
QSVLTQPPSVSGAPGQRVTISCSGSESNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 204 - protein, 247 aa, synthetic construct
QSVLTQPPSVSGAPGQRVTISCSGSSSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 205 - protein, 247 aa, synthetic construct
QSVLTQPPSVSGAPGQRVTISCSGSRSNIGDNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 206 - protein, 247 aa, synthetic construct
QSVLTQPPSVSGAPGQRVTISCSGSRSNIGENTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 207 - protein, 247 aa, synthetic construct
QSVLTQPPSVSGAPGQRVTISCSGSRSNIGSDTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 208 - protein, 247 aa, synthetic construct
QSVLTQPPSVSGAPGQRVTISCSGSRSNIGSETVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 209 - protein, 247 aa, synthetic construct
QSVLTQPPSVSGAPGQRVTISCSGSRSNIGSNDVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 210 - protein, 247 aa, synthetic construct
QSVLTQPPSVSGAPGQRVTISCSGSRSNIGSNTVDWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 211 - protein, 247 aa, synthetic construct
QSVLTQPPSVSGAPGQRVTISCSGSRSNIGSNTVEWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 212 - protein, 247 aa, synthetic construct
QSVLTQPPSVSGAPGQRVTISCSGSRSNIGSNTVKWYQQLPGTAPKLLIYYNDQDPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 213 - protein, 247 aa, synthetic construct
QSVLTQPPSVSGAPGQRVTISCSGSRSNIGSNTVKWYQQLPGTAPKLLIYYNDQEPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 214 - protein, 247 aa, synthetic construct
QSVLTQPPSVSGAPGQRVTISCSGSRSNIGSNTVKWYQQLPGTAPKLLIYYNDQRPDGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 215 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDEYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 216 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTDPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 217 - protein, 247 aa, synthetic construct
QSVLTQPPSVSGAPGQRVTISCSGSESNIGSNTVKWYQQLPGTAPKLLIYYNDQEPSGVPDRFSGSKSGTSASLAITGLQAEDEADYYCQSYDEYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 218 - protein, 247 aa, synthetic construct
QSVLTQPPSVSGAPGQRVTISCSGSESNIGSNDVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 219 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFESYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 220 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSEYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 221 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSDYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 222 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIEYDGSNKYYADSVKGRFTISRDNSKNTLYLQMNSLRAEDTAVYYCKTHGSHDNWGQGTMVTVSSHHHHHH

SEQ ID NO: 223 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIDYDGSNKYYADSVKGRFTISRDNSKNTLYLQMNSLRAEDTAVYYCKTHGSHDNWGQGTMVTVSSHHHHHH

SEQ ID NO: 224 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNDYYADSVKGRFTISRDNSKNTLYLQMNSLRAEDTAVYYCKTHGSHDNWGQGTMVTVSSHHHHHH

SEQ ID NO: 225 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNEYYADSVKGRFTISRDNSKNTLYLQMNSLRAEDTAVYYCKTHGSHDNWGQGTMVTVSSHHHHHH

SEQ ID NO: 226 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVEGRFTISRDNSKNTLYLQMNSLRAEDTAVYYCKTHGSHDNWGQGTMVTVSSHHHHHH

SEQ ID NO: 227 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSEDNWGQGTMVTVSSHHHHHH

SEQ ID NO: 228 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIEYDGSNKYYADSVEGRFTISRDNSKNTLYLQMNSLRAEDTAVYYCKTHGSHDNWGQGTMVTVSSHHHHHH

SEQ ID NO: 229 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIEYDGSNKYYADSVEGRFTISRDNSKNTLYLQMNSLRAEDTAVYYCKTHGSEDNWGQGTMVTVSSHHHHHH

SEQ ID NO: 230 - protein, 247 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 231 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 232 - protein, 137 aa, synthetic construct
QVQLQESGGGLVQTGGSLRLSCTTSGTIFSGYTMGWYRQAPGEQRELVAVISGGGDTNYADSVKGRFTISRDNTKDTMYLQMNSLKPEDTAVYYCYSREVTPPWKLYWGQGTQVTVSSAAAYPYDVPDYGSHHHHHH

SEQ ID NO: 233 - protein, 137 aa, synthetic construct
QVQLQESGGGLVQEGGSLRLSCAASERIFSTDVMGWYRQAAEKQRELVAVVSARGTTNYLDAVKGRFTISRDNARNTLTLQMNDLKPEDTASYYCYVRETTSPWRIYWGQGTQVTVSSAAAYPYDVPDYGSHHHHHH

SEQ ID NO: 234 - protein, 138 aa, synthetic construct
QVQLQESGGGLVQAGGSLRLSCAASGSIFSANAMGWYRQAPGKQRELVAVISSGGSTNYADSVKGRFTISRDNAKNTVYLQMNSLKPEDTAVYYCMYSGSYYYTPNDYWGQGTQVTVSSAAAYPYDVPDYGSHHHHHH

SEQ ID NO: 235 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 236 - protein, 138 aa, synthetic construct
EVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSSAAAYPYDVPDYGSHHHHHH

SEQ ID NO: 237 - protein, 138 aa, synthetic construct
EVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSSAAAYPYDVPDYGSHHHHHH

SEQ ID NO: 238 - protein, 138 aa, synthetic construct
EVQLLESGGGLVQPGGSLRLSCAASGSIFSANAMGWVRQAPGKGLEWVSVISSGGSTNYADSVKGRFTISRDNSKNTLYLQMNSLRAEDTAVYYCMYSGSYYYTPNDYWGQGTLVTVSSAAAYPYDVPDYGSHHHHHH

SEQ ID NO: 239 - protein, 138 aa, synthetic construct
QVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSSAAAYPYDVPDYGSHHHHHH

SEQ ID NO: 240 - protein, 138 aa, synthetic construct
EVQLLESGGGLVQPGGSLRLSCAASGSIFSANAMGWYRQAPGKGRELVAVISSGGSTNYADSVKGRFTISRDNSKNTVYLQMNSLRAEDTAVYYCMYSGSYYYTPNDYWGQGTLVTVSSAAAYPYDVPDYGSHHHHHH

SEQ ID NO: 241 - protein, 138 aa, synthetic construct
QVQLLESGGGLVQAGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSSAAAYPYDVPDYGSHHHHHH

SEQ ID NO: 242 - protein, 137 aa, synthetic construct
EVQLLESGGGLVQPGGSLRLSCAASERIFSTDVMGWYRQAPGKQRELVAVVSARGTTNYLDAVKGRFTISRDNSKNTLYLQMNSLRAEDTAVYYCYVRETTSPWRIYWGQGTLVTVSSAAAYPYDVPDYGSHHHHHH

SEQ ID NO: 243 - protein, 137 aa, synthetic construct
EVQLLESGGGLVQPGGSLRLSCAASERIFSTDVMGWYRQAPGKGLELVAVVSARGTTNYLDAVKGRFTISRDNSKNTLYLQMNSLRAEDTAVYYCYVRETTSPWRIYWGQGTLVTVSSAAAYPYDVPDYGSHHHHHH

SEQ ID NO: 244 - protein, 137 aa, synthetic construct
EVQLLESGGGLVQPGGSLRLSCAASERIFSTDVMGWVRQAPGKGLEWVSVVSARGTTNYLDAVKGRFTISRDNSKNTLYLQMNSLRAEDTAVYYCYVRETTSPWRIYWGQGTLVTVSSAAAYPYDVPDYGSHHHHHH

SEQ ID NO: 245 - protein, 137 aa, synthetic construct
QVQLLESGGGLVQPGGSLRLSCAASERIFSTDVMGWYRQAPGKQRELVAVVSARGTTNYLDAVKGRFTISRDNSKNTLYLQMNSLRAEDTAVYYCYVRETTSPWRIYWGQGTLVTVSSAAAYPYDVPDYGSHHHHHH

SEQ ID NO: 246 - protein, 137 aa, synthetic construct
EVQLLESGGGLVQPGGSLRLSCAASERIFSTDVMGWYRQAPGKGRELVAVVSARGTTNYLDSVKGRFTISRDNSKNTLYLQMNSLRAEDTAVYYCYVRETTSPWRIYWGQGTLVTVSSAAAYPYDVPDYGSHHHHHH

SEQ ID NO: 247 - protein, 137 aa, synthetic construct
QVQLLESGGGLVQEGGSLRLSCAASERIFSTDVMGWYRQAAGKQRELVAVVSARGTTNYLDAVKGRFTISRDNSKNTLYLQMNSLRAEDTASYYCYVRETTSPWRIYWGQGTLVTVSSAAAYPYDVPDYGSHHHHHH

SEQ ID NO: 248 - protein, 137 aa, synthetic construct
EVQLLESGGGLVQPGGSLRLSCAASERIFSTDVMGWYRQAPGKGLELVAVVSARGTTNYLDSVKGRFTISRDNSKNTLYLQMNSLRAEDTAVYYCYVRETTSPWRIYWGQGTLVTVSSAAAYPYDVPDYGSHHHHHH

SEQ ID NO: 249 - protein, 137 aa, synthetic construct
EVQLLESGGGLVQPGGSLRLSCATSGTIFSGYTMGWYRQAPGKQRELVAVISGGGDTNYADSVKGRFTISRDNSKDTMYLQMNSLRAEDTAVYYCYSREVTPPWKLYWGQGTLVTVSSAAAYPYDVPDYGSHHHHHH

SEQ ID NO: 250 - protein, 137 aa, synthetic construct
EVQLLESGGGLVQPGGSLRLSCATSGTIFSGYTMGWYRQAPGKGLELVAVISGGGDTNYADSVKGRFTISRDNSKDTMYLQMNSLRAEDTAVYYCYSREVTPPWKLYWGQGTLVTVSSAAAYPYDVPDYGSHHHHHH

SEQ ID NO: 251 - protein, 137 aa, synthetic construct
EVQLLESGGGLVQPGGSLRLSCAASGTIFSGYTMGWVRQAPGKGLEWVSVISGGGDTNYADSVKGRFTISRDNSKNTLYLQMNSLRAEDTAVYYCYSREVTPPWKLYWGQGTLVTVSSAAAYPYDVPDYGSHHHHHH

SEQ ID NO: 252 - protein, 137 aa, synthetic construct
QVQLLESGGGLVQPGGSLRLSCATSGTIFSGYTMGWYRQAPGKQRELVAVISGGGDTNYADSVKGRFTISRDNSKDTMYLQMNSLRAEDTAVYYCYSREVTPPWKLYWGQGTLVTVSSAAAYPYDVPDYGSHHHHHH

SEQ ID NO: 253 - protein, 137 aa, synthetic construct
EVQLLESGGGLVQPGGSLRLSCATSGTIFSGYTMGWYRQAPGKQRELVAVISGGGDTNYADSVKGRFTISRDNSKNTMYLQMNSLRAEDTAVYYCYSREVTPPWKLYWGQGTLVTVSSAAAYPYDVPDYGSHHHHHH

SEQ ID NO: 254 - protein, 137 aa, synthetic construct
EVQLLESGGGLVQPGGSLRLSCATSGTIFSGYTMGWYRQAPGKGRELVAVISGGGDTNYADSVKGRFTISRDNSKNTMYLQMNSLRAEDTAVYYCYSREVTPPWKLYWGQGTLVTVSSAAAYPYDVPDYGSHHHHHH

SEQ ID NO: 255 - protein, 137 aa, synthetic construct
QVQLLESGGGLVQTGGSLRLSCATSGTIFSGYTMGWYRQAPGKQRELVAVISGGGDTNYADSVKGRFTISRDNSKDTMYLQMNSLRAEDTAVYYCYSREVTPPWKLYWGQGTLVTVSSAAAYPYDVPDYGSHHHHHH

SEQ ID NO: 256 - protein, 137 aa, synthetic construct
EVQLLESGGGLVQPGGSLRLSCATSGTIFSGYTMGWYRQAPGKGLELVAVISGGGDTNYADSVKGRFTISRDNSKNTMYLQMNSLRAEDTAVYYCYSREVTPPWKLYWGQGTLVTVSSAAAYPYDVPDYGSHHHHHH

SEQ ID NO: 257 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKALIYSASFRYSGVPSRFSGSGSGTDFTLTISSLQPEDFATYYCQQYYTYPYTFGGGTKVEIKHHHHHH

SEQ ID NO: 258 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIKHHHHHH

SEQ ID NO: 259 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIKHHHHHH

SEQ ID NO: 260 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKSSEKLWANVAWYQQKPGKAPKALIYSASFRYSGVPSRFSGSGSGTDFTLTISSLQPEDFATYYCQQYYTYPYTFGGGTKVEIKHHHHHH

SEQ ID NO: 261 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKSSEKLWANVAWYQQKPGKAPKSLIYSASFRYSGVPSRFSGSGSGTDFTLTISSLQPEDFATYYCQQYYTYPYTFGGGTKVEIKHHHHHH

SEQ ID NO: 262 - protein, 214 aa, synthetic construct
DIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - protein, 214 aa, synthetic construct
DIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 264 - protein, 214 aa, synthetic construct
DIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 363 aa, synthetic construct
DIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSS

SEQ ID NO: 266 - protein, 363 aa, synthetic construct
DIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSS

SEQ ID NO: 267 - protein, 363 aa, synthetic construct
DIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSS

SEQ ID NO: 268 - protein, 363 aa, synthetic construct
DIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSS

SEQ ID NO: 269 - protein, 214 aa, synthetic construct
DIQMTQSPSSLSASVGDRVTITCKSSEKLWANVAWYQQKPGKAPKSLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - protein, 214 aa, synthetic construct
DIQMTQSPSSLSASVGDRVTITCKSSEKLWANVAWYQQKPGKAPKLLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 214 aa, synthetic construct
DIQMTQSPSSLSASVGDRVTITCKAREKLWSAVAWYQQKPGKAPKLLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKSSEKLWANVAWYQQKPGKAPKALIYSASFRYSGVPSRFSGSGSGTDFTLTISSLQPEDFATYYCQQYYTYPYTFGGGTKVEIK

SEQ ID NO: 273 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKSSEKLWANVAWYQQKPGKAPKSLIYSASFRYSGVPSRFSGSGSGTDFTLTISSLQPEDFATYYCQQYYTYPYTFGGGTKVEIK

SEQ ID NO: 274 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKSSEKLWANVAWYQQKPGKAPKLLIYSASFRYSGVPSRFSGSGSGTDFTLTISSLQPEDFATYYCQQYYTYPYTFGGGTKVEIK

SEQ ID NO: 275 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKSSEKLWANVAWYQQKPGKAPKSLIYSASFRYSGVPSRFSGSGSGTDFTLTISSLQPEDFATYYCQQYYTYPYTFGCGTKVEIK

SEQ ID NO: 276 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKSSEKLWANVAWYQQKPGKAPKLLIYSASFRYSGVPSRFSGSGSGTDFTLTISSLQPEDFATYYCQQYYTYPYTFGCGTKVEIK

SEQ ID NO: 277 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKSSEKLWANVAWYQQKPGKCPKALIYSASFRYSGVPSRFSGSGSGTDFTLTISSLQPEDFATYYCQQYYTYPYTFGGGTKVEIK

SEQ ID NO: 278 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKAREKLWSAVAWYQQKPGKAPKLLIYSASFRYSGVPSRFSGSGSGTDFTLTISSLQPEDFATYYCQQYYTYPYTFGGGTKVEIK

SEQ ID NO: 279 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKLLIYSASFRYSGVPSRFSGSGSGTDFTLTISSLQPEDFATYYCQQYYTYPYTFGGGTKVEIK

SEQ ID NO: 280 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKLLIYSASFRYSGVPSRFSGSGSGTDFTLTISSLQPEDFATYYCQQYYTYPYTFGCGTKVEIK

SEQ ID NO: 281 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 282 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIK

SEQ ID NO: 283 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIK

SEQ ID NO: 284 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIK

SEQ ID NO: 285 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIK

SEQ ID NO: 286 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 287 - protein, 642 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 288 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 289 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIK

SEQ ID NO: 290 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIK

SEQ ID NO: 291 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIK

SEQ ID NO: 292 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIK

SEQ ID NO: 293 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIK

SEQ ID NO: 294 - protein, 659 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 295 - protein, 659 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIK

SEQ ID NO: 296 - protein, 659 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIK

SEQ ID NO: 297 - protein, 659 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIK

SEQ ID NO: 298 - protein, 659 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIK

SEQ ID NO: 299 - protein, 659 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIK

SEQ ID NO: 300 - protein, 662 aa, synthetic construct
MDMRVPAQLLGLLLLWLRGARCVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPALFKSSFPPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 301 - protein, 214 aa, synthetic construct; identical to SEQ ID NO: 264

SEQ ID NO: 302 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGCGTKVEIK

SEQ ID NO: 303 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKASQNVGTNVGWYQQKPGKCPKALIYSASFRYSGVPSRFSGSGSGTDFTLTISSLQPEDFATYYCQQYYTYPYTFGGGTKVEIK

SEQ ID NO: 304 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKALIYSASFRYSGVPSRFSGSGSGTDFTLTISSLQPEDFATYYCQQYYTYPYTFGCGTKVEIK

SEQ ID NO: 305 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIK

SEQ ID NO: 306 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIK

SEQ ID NO: 307 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIK

SEQ ID NO: 308 - protein, 541 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSS

SEQ ID NO: 309 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIK

SEQ ID NO: 310 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIK

SEQ ID NO: 311 - protein, 811 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIK

SEQ ID NO: 312 - protein, 811 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIK

SEQ ID NO: 313 - protein, 811 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIK

SEQ ID NO: 314 - protein, 811 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIK

SEQ ID NO: 315 - protein, 691 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSS

SEQ ID NO: 316 - protein, 691 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSS

SEQ ID NO: 317 - protein, 792 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 318 - protein, 676 aa, synthetic construct
EVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIKSGGPGPAGLYAQPGSGGGGSGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSTFKFYMPKKATELKHLQCLEEELKPLEEVLNLAQSKNFHLRPRDLISNINVIVLELKGSETTFMCEYADETATIVEFLNRWITFCQSIISTLTGGSSSTKKTQLQLEHLLLDLQMILNGINNYKNPKLTRMLSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 319 - protein, 676 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIKSGGPGPAGLYAQPGSGGGGSGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSTFKFYMPKKATELKHLQCLEEELKPLEEVLNLAQSKNFHLRPRDLISNINVIVLELKGSETTFMCEYADETATIVEFLNRWITFCQSIISTLTGGSSSTKKTQLQLEHLLLDLQMILNGINNYKNPKLTRMLSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 320 - protein, 676 aa, synthetic construct
EVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIKSGGPGPAGLYAQPGSGGGGSGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSTFKFYMPKKATELKHLQCLEEELKPLEEVLNLAQSKNFHLRPRDLISNINVIVLELKGSETTFMCEYADETATIVEFLNRWITFCQSIISTLTGGSSSTKKTQLQLEHLLLDLQMILNGINNYKNPKLTRMLSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 321 - protein, 676 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIKSGGPGPAGLYAQPGSGGGGSGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSTFKFYMPKKATELKHLQCLEEELKPLEEVLNLAQSKNFHLRPRDLISNINVIVLELKGSETTFMCEYADETATIVEFLNRWITFCQSIISTLTGGSSSTKKTQLQLEHLLLDLQMILNGINNYKNPKLTRMLSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 322 - protein, 556 aa, synthetic construct
EVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSSSGGPGPAGLYAQPGSGGGGSGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSTFKFYMPKKATELKHLQCLEEELKPLEEVLNLAQSKNFHLRPRDLISNINVIVLELKGSETTFMCEYADETATIVEFLNRWITFCQSIISTLTGGSSSTKKTQLQLEHLLLDLQMILNGINNYKNPKLTRMLSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 323 - protein, 556 aa, synthetic construct
EVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSSSGGPGPAGLYAQPGSGGGGSGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSTFKFYMPKKATELKHLQCLEEELKPLEEVLNLAQSKNFHLRPRDLISNINVIVLELKGSETTFMCEYADETATIVEFLNRWITFCQSIISTLTGGSSSTKKTQLQLEHLLLDLQMILNGINNYKNPKLTRMLSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 324 - protein, 657 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCSGGPGPAGLYAQPGSGGGGSGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSTFKFYMPKKATELKHLQCLEEELKPLEEVLNLAQSKNFHLRPRDLISNINVIVLELKGSETTFMCEYADETATIVEFLNRWITFCQSIISTLTGGSSSTKKTQLQLEHLLLDLQMILNGINNYKNPKLTRMLSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 325 - protein, 640 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 326 - protein, 659 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKALIYSASFRYSGVPSRFSGSGSGTDFTLTISSLQPEDFATYYCQQYYTYPYTFGCGTKVEIK

SEQ ID NO: 327 - protein, 659 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIK

SEQ ID NO: 328 - protein, 659 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIK

SEQ ID NO: 329 - protein, 659 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIK

SEQ ID NO: 330 - protein, 659 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIK

SEQ ID NO: 331 - protein, 539 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSS

SEQ ID NO: 332 - protein, 463 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 463 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 811 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIKGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 335 - protein, 811 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 336 - protein, 811 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIKGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 337 - protein, 811 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIKGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 338 - protein, 691 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSSGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 339 - protein, 691 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSGGGGSGGGGSGGGGSGGGGSGGGGSGGGGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSSGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 340 - protein, 811 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIKGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 341 - protein, 811 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 342 - protein, 811 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIKGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 343 - protein, 811 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIKGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 344 - protein, 691 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSSGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 345 - protein, 691 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSSGGGGSGGGGSGGGGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 346 - protein, 696 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIKGGGGSGGGGSGGGGSGGGGSGGGGSGGGGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSS

SEQ ID NO: 347 - protein, 696 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSS

SEQ ID NO: 348 - protein, 696 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIKGGGGSGGGGSGGGGSGGGGSGGGGSGGGGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSS

SEQ ID NO: 349 - protein, 696 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIKGGGGSGGGGSGGGGSGGGGSGGGGSGGGGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSS

SEQ ID NO: 350 - protein, 696 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGCGTKVEIKGGGGSGGGGSGGGGSGGGGSGGGGSGGGGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSS

SEQ ID NO: 351 - protein, 696 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIKGGGGSGGGGSGGGGSGGGGSGGGGSGGGGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSS

SEQ ID NO: 352 - protein, 696 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIKGGGGSGGGGSGGGGSGGGGSGGGGSGGGGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSS

SEQ ID NO: 353 - protein, 696 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIKGGGGSGGGGSGGGGSGGGGSGGGGSGGGGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSS

SEQ ID NO: 354 - protein, 6 aa, synthetic construct
  source 1..6: note = 6xHis tag
HHHHHH

SEQ ID NO: 355 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGCGTKVEIK

SEQ ID NO: 356 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKASQNVGTNVGWYQQKPGKCPKALIYSASFRYSGVPSRFSGSGSGTDFTLTISSLQPEDFATYYCQQYYTYPYTFGGGTKVEIK

SEQ ID NO: 357 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKALIYSASFRYSGVPSRFSGSGSGTDFTLTISSLQPEDFATYYCQQYYTYPYTFGCGTKVEIK

SEQ ID NO: 358 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKAREKLWSAVAWYQQKPGKCPKALIYSASFRYSGVPSRFSGSGSGTDFTLTISSLQPEDFATYYCQQYYTYPYTFGGGTKVEIK

SEQ ID NO: 359 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKCLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGCGTKVEIK

SEQ ID NO: 360 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIK

SEQ ID NO: 361 - protein, 427 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLLESGGGLVQPGGSLRLSCAASGSIFSANAMGWYRQAPGKQRELVAVISSGGSTNYADSVKGRFTISRDNSKNTVYLQMNSLRAEDTAVYYCMYSGSYYYTPNDYWGQGTLVTVSS

SEQ ID NO: 362 - protein, 528 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 363 - protein, 642 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 364 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIKHHHHHH

SEQ ID NO: 365 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKAREKLWSAVAWYQQKPGKAPKALIYSASFRYSGVPSRFSGSGSGTDFTLTISSLQPEDFATYYCQQYYTYPYTFGGGTKVEIKHHHHHH

SEQ ID NO: 366 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIKHHHHHH

SEQ ID NO: 367 - protein, 609 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 368 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESSLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 369 - protein, 935 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSS

SEQ ID NO: 370 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGCGTKVTVLGGGGSGGGGSGGGGSQVQLVESGGGVVQPGRSLRLSCAASGFTFSSYGMHWVRQAPGKCLEWVAFIRYDGSNKYYADSVKGRFTISRDNSKNTLYLQMNSLRAEDTAVYYCKTHGSHDNWGQGTMVTVSS

SEQ ID NO: 371 - protein, 930 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTCPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGCGTMVTVSS

SEQ ID NO: 372 - protein, 553 aa, synthetic construct; identical to SEQ ID NO: 49

SEQ ID NO: 373 - protein, 444 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGQVQLQESGGGLVQTGGSLRLSCTTSGTIFSGYTMGWYRQAPGEQRELVAVISGGGDTNYADSVKGRFTISRDNTKDTMYLQMNSLKPEDTAVYYCYSREVTPPWKLYWGQGTQVTVSSAAAYPYDVPDYGSHHHHHH

SEQ ID NO: 374 - protein, 444 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGQVQLQESGGGLVQEGGSLRLSCAASERIFSTDVMGWYRQAAEKQRELVAVVSARGTTNYLDAVKGRFTISRDNARNTLTLQMNDLKPEDTASYYCYVRETTSPWRIYWGQGTQVTVSSAAAYPYDVPDYGSHHHHHH

SEQ ID NO: 375 - protein, 445 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGQVQLQESGGGLVQAGGSLRLSCAASGSIFSANAMGWYRQAPGKQRELVAVISSGGSTNYADSVKGRFTISRDNAKNTVYLQMNSLKPEDTAVYYCMYSGSYYYTPNDYWGQGTQVTVSSAAAYPYDVPDYGSHHHHHH

SEQ ID NO: 376 - protein, 557 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGMKGLPGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 377 - protein, 557 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKLLIYSASFRYSGVPSRFSGSGSGTDFTLTISSLQPEDFATYYCQQYYTYPYTFGGGTKVEIKHHHHHHEPEA

SEQ ID NO: 378 - protein, 557 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKGLIYSASFRYSGVPSRFSGSGSGTDFTLTISSLQPEDFATYYCQQYYTYPYTFGGGTKVEIKHHHHHHEPEA

SEQ ID NO: 379 - protein, 557 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 380 - protein, 1027 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHHEPEA

SEQ ID NO: 381 - protein, 1022 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 382 - protein, 1336 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSTFKFYMPKKATELKHLQCLEEELKPLEEVLNLAQSKNFHLRPRDLISNINVIVLELKGSETTFMCEYADETATIVEFLNRWITFCQSIISTLTGGSSSTKKTQLQLEHLLLDLQMILNGINNYKNPKLTRMLSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHH

SEQ ID NO: 383 - protein, 817 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKSGGPGPAGMKGLPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQHHHHHH

SEQ ID NO: 384 - protein, 817 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQGGGGSGGGGSGGGGSGGGGSGGGGSGGGGSSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 385 - protein, 817 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 386 - protein, 682 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSTFKFYMPKKATELKHLQCLEEELKPLEEVLNLAQSKNFHLRPRDLISNINVIVLELKGSETTFMCEYADETATIVEFLNRWITFCQSIISTLTGGSSSTKKTQLQLEHLLLDLQMILNGINNYKNPKLTRMLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 387 - protein, 682 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKSGGPGPAGMKGLPGSGGGGSGGGGSGGGGSGGGGSGGGGSGGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSTFKFYMPKKATELKHLQCLEEELKPLEEVLNLAQSKNFHLRPRDLISNINVIVLELKGSETTFMCEYADETATIVEFLNRWITFCQSIISTLTGGSSSTKKTQLQLEHLLLDLQMILNGINNYKNPKLTRMLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 388 - protein, 553 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKSLIYSASFRYSGVPSRFSGSGSGTDFTLTISSLQPEDFATYYCQQYYTYPYTFGGGTKVEIKHHHHHH

SEQ ID NO: 389 - protein, 553 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGQAPRLLIYSASFRYSGVPSRFSGSGSGTDFTLTISSLQPEDFATYYCQQYYTYPYTFGGGTKVEIKHHHHHH

SEQ ID NO: 390 - protein, 667 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 391 - protein, 667 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 392 - protein, 667 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGMKGLPGSGGGGSGGGGSGGGGSGGGGSGGGGSGGGGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKSGGPGPAGMKGLPGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKHHHHHH

SEQ ID NO: 393 - protein, 665 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPGPAGMKGLPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGMKGLPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 394 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 395 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 396 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIK

SEQ ID NO: 397 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIK

SEQ ID NO: 398 - protein, 427 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSS

SEQ ID NO: 399 - protein, 426 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLLESGGGLVQPGGSLRLSCAASERIFSTDVMGWYRQAPGKQRELVAVVSARGTTNYLDAVKGRFTISRDNSKNTLYLQMNSLRAEDTAVYYCYVRETTSPWRIYWGQGTLVTVSS

SEQ ID NO: 400 - protein, 811 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 401 - protein, 811 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 402 - protein, 811 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIK

SEQ ID NO: 403 - protein, 811 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIK

SEQ ID NO: 404 - protein, 691 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSS

SEQ ID NO: 405 - protein, 690 aa, synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPGPAGLYAQPGSEVQLLESGGGLVQPGGSLRLSCAASERIFSTDVMGWYRQAPGKQRELVAVVSARGTTNYLDAVKGRFTISRDNSKNTLYLQMNSLRAEDTAVYYCYVRETTSPWRIYWGQGTLVTVSS

SEQ ID NO: 406 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 407 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 408 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIK

SEQ ID NO: 409 - protein, 661 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIK

SEQ ID NO: 410 - protein, 541 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSS

SEQ ID NO: 411 - protein, 540 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLLESGGGLVQPGGSLRLSCAASERIFSTDVMGWYRQAPGKQRELVAVVSARGTTNYLDAVKGRFTISRDNSKNTLYLQMNSLRAEDTAVYYCYVRETTSPWRIYWGQGTLVTVSS

SEQ ID NO: 412 - protein, 925 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 413 - protein, 925 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKAREKLWSAVAWYQQKPGKAPKSLIYSASFRYSGVPSRFSGSGSGTDFTLTISSLQPEDFATYYCQQYYTYPYTFGGGTKVEIK

SEQ ID NO: 414 - protein, 925 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIK

SEQ ID NO: 415 - protein, 925 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKVTEKVWGNVAWYQQKPGKAPISLIYSPSLRKSGVPSRFSGSGSGTDFTLTISSLQPEDFATYYCQQYYTYPYTFGGGTKVEIK

SEQ ID NO: 416 - protein, 805 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLLESGGGLVQPGGSLRLSCAASGSIFSANAMGWYRQAPGKGLELVAVISSGGSTNYADSVKGRFTISRDNSKNTVYLQMNSLRAEDTAVYYCMYSGSYYYTPNDYWGQGTLVTVSS

SEQ ID NO: 417 - protein, 804 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGPGPAGLYAQPGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEYQGGGGSGGGGSGGGGSGGGGSGGGGSGGGGSSGGPGPAGLYAQPGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGGGGSGGGGSSGGPGPAGLYAQPGSEVQLLESGGGLVQPGGSLRLSCAASERIFSTDVMGWYRQAPGKQRELVAVVSARGTTNYLDAVKGRFTISRDNSKNTLYLQMNSLRAEDTAVYYCYVRETTSPWRIYWGQGTLVTVSS

SEQ ID NO: 418 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGGGSGGGGSDIQMTQSPSSLSASVGDRVTITCKASQNVGTNVGWYQQKPGKAPKSLIYSASFRYSGVPSRFSGSGSGTDFTLTISSLQPEDFATYYCQQYYTYPYTFGGGTKVEIKHHHHHH

SEQ ID NO: 419 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKHHHHHH

SEQ ID NO: 420 - protein, 245 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSSGGPGPAGLYAQPGSDIQMTQSPSSLSASVGDRVTITCKASQNVGTNVGWYQQKPGKAPKSLIYSASFRYSGVPSRFSGSGSGTDFTLTISSLQPEDFATYYCQQYYTYPYTFGGGTKVEIKHHHHHH

SEQ ID NO: 421 - protein, 612 aa, synthetic construct
LVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPGPAGMKGLPGSLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHH

SEQ ID NO: 422 - protein, 1030 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQGGGGSGGGGSGGSLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQGGGGSGGGGSGGSLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHH

SEQ ID NO: 423 - protein, 466 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGSGGPGPAGMKGLPGSGGGGSGGGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKGGGGSGGGSGGPGPAGMKGLPGSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 424 - protein, 426 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 425 - protein, 426 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 426 - protein, 428 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPPLAQKLKSSPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPPLAQKLKSSPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 427 - protein, 425 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGLYAQPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPGPAGLYAQPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 428 - protein, 425 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPALFKSSFPPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 429 - protein, 427 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPPLAQKLKSSPGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKESGGPPLAQKLKSSPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 430 - protein, 728 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSCDLPQTHNLRNKRALTLLVQMRRLSPLSCLKDRKDFGFPQEKVDAQQIKKAQAIPVLSELTQQILNIFTSKDSSAAWNTTLLDSFCNDLHQQLNDLQGCLMQQVGVQEFPLTQEDALLAVRKYFHRITVYLREKKHSPCAWEVVRAEVWRALSSSANVLGRLREEKSGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 431 - protein, 947 aa, synthetic construct
LVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHH

SEQ ID NO: 432 - protein, 1574 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQGGGGSGGGGSGGSLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQGGGGSGGGGSGGSLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQGGGGSGGGGSGGSLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHH

SEQ ID NO: 433 - protein, 745 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGSGGPGPAGMKGLPGSGGGGSGGGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCGGGGSGGGSGGPGPAGMKGLPGSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGSGGPGPAGMKGLPGSGGGGSGGGSHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRCGGGGSGGGSGGPGPAGMKGLPGSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 434 - protein, 936 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 435 - protein, 1405 aa, synthetic construct
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALASGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 436 - protein, 1405 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSSGGPGPAGMKGLPGSEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHH

SEQ ID NO: 437 - protein, 1048 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 438 - protein, 1048 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSSGGPGPAGMKGLPGSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKHHHHHH

SEQ ID NO: 439 - protein, 936 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLSGGPGPAGMKGLPGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSHHHHHH

SEQ ID NO: 440 - protein, 817 aa, synthetic construct
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSGGPGPAGMKGLPGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSASGGPGPAGMKGLPGSGGGGSGGGGSGGGGSGGGGSGGGGSGGGGSQVQLQESGGGLVQAGGSLRLSCAASGRTFSSVYDMGWFRQAPGKDREFVARITESARNTRYADSVRGRFTISRDNAKNTVYLQMNNLELEDAAVYYCAADPQTVVVGTPDYWGQGTQVTVSSHHHHHH

SEQ ID NO: 441 - protein, 547 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGPALFKSSFPPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGGSGGGGSGGGGSGGGGSGGGGSSGGPALFKSSFPPGSEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGCGTTVTVSSGGGGSGGGGSGGGGSDIQMTQSPSSLSASVGDRVTITCKVTEKVWGNVAWYQQKPGKCPISLIYSPSLRKSGVPSRFSGSGSGTDFTLTISSLQPEDFATYYCQQYYTYPYTFGGGTKVEIK

SEQ ID NO: 442 - protein, 5 aa, unidentified
  VARIANT 3: note = Any amino acid
  source 1..5: note = Sortase A cleavage site
LPXTG

SEQ ID NO: 443 - protein, 25 aa, synthetic construct
  VARIANT 6..25: note = GGGGS repeats may be deleted
GGGGSGGGGSGGGGSGGGGSGGGGS

SEQ ID NO: 444 - protein, 20 aa, synthetic construct
  VARIANT 5..20: note = GGGS repeats may be deleted
GGGSGGGSGGGSGGGSGGGS

SEQ ID NO: 445 - protein, 7 aa, unidentified
  source 1..7: note = protease-cleavable sequence
GPLGVRG

SEQ ID NO: 446 - protein, 8 aa, unidentified
  source 1..8: note = protease-cleavable sequence
IPVSLRSG

SEQ ID NO: 447 - protein, 8 aa, unidentified
  source 1..8: note = protease-cleavable sequence
VPLSLYSG

SEQ ID NO: 448 - protein, 10 aa, unidentified
  source 1..10: note = protease-cleavable sequence
SGESPAYYTA

SEQ ID NO: 449 - protein, 5 aa, synthetic construct
GGGGS

SEQ ID NO: 450 - protein, 6 aa, synthetic construct
GSGSGS

SEQ ID NO: 451 - protein, 13 aa, synthetic construct
GSGGGSGGGSGGT

SEQ ID NO: 452 - protein, 15 aa, synthetic construct
GGGGSGGGGSGGGGS

SEQ ID NO: 453 - protein, 20 aa, synthetic construct
GGGGSGGGGSGGGGSGGGGS

(19) United States
(12) Patent Application Publication
(10) Pub. No.: US 2026/0250345 A1
(43) Pub. Date: Aug. 27, 2026

(54) ACTIVATABLE CYTOKINE POLYPEPTIDES AND METHODS OF USE THEREOF

(71) Applicant: Werewolf Therapeutics, Inc., Watertown, MA (US)

(72) Inventors: William WINSTON, West Newton, MA (US); Heather BRODKIN, West Newton, MA (US); Cynthia SEIDEL-DUGAN, Belmont, MA (US); Daniel HICKLIN, Boston, MA (US); Jose Andres SALMERON-GARCIA, Acton, MA (US)

(21) Appl. No.: 19/562,226

(22) Filed: Mar. 10, 2026

Related U.S. Application Data
(63) Continuation of application No. 18/601,474, filed on Mar. 11, 2024, now Pat. No. 12,617,829.
Application No. 18/601,474 is a continuation of application No. 18/312,245, filed on May 4, 2023.
Application No. 18/312,245 is a continuation of application No. 17/028,643, filed on Sep. 22, 2020, now abandoned.
Application No. 17/028,643 is a continuation-in-part of application No. PCT/US2019/032320, filed on May 14, 2019.
(60) Provisional application No. 62/671,225, filed on May 14, 2018.
(60) Provisional application No. 62/756,504, filed on Nov. 6, 2018.
(60) Provisional application No. 62/756,507, filed on Nov. 6, 2018.
(60) Provisional application No. 62/756,515, filed on Nov. 6, 2018.
(60) Provisional application No. 62/935,605, filed on Nov. 14, 2019.

(51) Int. Cl. C07K 14/555; C07K 14/54; C07K 14/55; C07K 14/715; C07K 16/28

(52) U.S. Cl. CPC C07K 14/555; C07K 14/7156; C07K 14/5434; C07K 14/55; C07K 16/2818; C07K 16/2827; C07K 2319/31

(57) ABSTRACT

The disclosure features fusion proteins that are conditionally active variants of a cytokine of interest. In one aspect, the full-length polypeptides of the invention have reduced or minimal cytokine-receptor activating activity even though they contain a functional cytokine polypeptide. Upon activation, e.g., by cleavage of a linker that joins a blocking moiety, e.g. a steric blocking polypeptide, in sequence to the active cytokine, the cytokine can bind its receptor and effect signaling. Typically, the fusion proteins further comprise an in vivo half-life extension element, which may be cleaved from the cytokine in the tumor microenvironment.

### RELATED APPLICATIONS

This application is a continuation of Ser. No. 18/601,474, filed Mar. 11, 2024, which is a continuation of Ser. No. 18/312,245, filed on May 4, 2023, which is a continuation of Ser. No. 17/208,643, filed Sep. 22, 2020, which is a is a continuation-in-part of PCT/US2019/032320, filed on May 14, 2019, which claims the benefit of U.S. Provisional Application 62/671,225, filed on May 14, 2018, U.S. Provisional Application No. 62/756,504, filed on Nov. 6, 2018, U.S. Provisional Application No. 62/756,507, filed on Nov. 6, 2018, and U.S. Provisional Application No. 62/756,515, filed on Nov. 6, 2018; and claims the benefit of U.S. Provisional Application No. 62/935,605, filed on Nov. 14, 2019, each of which are incorporated herein by reference.

### SEQUENCE LISTING

The instant application contains a Sequence Listing which has been submitted electronically in XML format and is hereby incorporated by reference in its entirety. Said XML file, created on Mar. 18, 2026, is named 761146_200013_SL.xml and is 618267 bytes in size.

### BACKGROUND

The development of mature immunocompetent lymphoid cells from less-committed precursors, their subsequent antigen-driven immune responses, and the suppression of these and unwanted autoreactive responses are highly dependent and regulated by cytokines (including interleukin-2 [IL-2], IL-4, IL-7, IL-9, IL-15, and IL-21) that utilize receptors in the common γ-chain (γc) family (Rochman et al., 2009) and family members including IL-12, 18 and 23. IL-2 is essential for thymic development of Treg cells and critically regulates several key aspects of mature peripheral Treg and antigen-activated conventional T cells. Because of its potent T cell growth factor activity in vitro, IL-2 has been extensively studied in part because this activity offered a potential means to directly boost immunity, e.g., in cancer and AIDS-HIV patients, or a target to antagonize unwanted responses, e.g., transplantation rejection and autoimmune diseases. Although in vitro studies with IL-2 provided a strong rationale for these studies, the function of IL-2 in vivo is clearly much more complex as first illustrated in IL-2-deficient mice, where a rapid lethal autoimmune syndrome, not lack of immunity, was observed (Sadlack et al., 1993, 1995). Similar observations were later made when the gene encoding IL-2Rα (II2ra) and IL-2Rβ (II2rb) were individually ablated (Suzuki et al., 1995; Willerford et al., 1995).

The present invention refers to conditionally active and/or targeted cytokines for use in the treatment of cancer and other diseases dependent on immune up or down regulation. For example, the antitumoral activity of some cytokines is well known and described and some cytokines have already been used therapeutically in humans. Cytokines such as interleukin-2 (IL-2) and interferon α (IFNα) have shown positive antitumoral activity in patients with different types of tumors, such as kidney metastatic carcinoma, hairy cell leukemia, Kaposi sarcoma, melanoma, multiple myeloma, and the like. Other cytokines like IFNβ, the Tumor Necrosis Factor (TNF) α, TNFβ, IL-1, 4, 6, 12, 15 and the CSFs have shown a certain antitumoral activity on some types of tumors and therefore are the object of further studies.

### SUMMARY

Provided herein are therapeutic proteins, nucleic acids that encode the proteins, and compositions and methods of using the proteins and nucleic acids for the treatment of a disease or disorder, such as proliferative disease, a tumorous disease, an inflammatory disease, an immunological disorder, an autoimmune disease, an infectious disease, a viral disease, an allergic reaction, a parasitic reaction, graft-versus-host disease and the like. In certain embodiments, the protein is one or more of, including any combinations, SEQ ID NOs.: 193-271 and the protein referred to herein as:

ACP200
ACP201
ACP202
ACP203
ACP204
ACP205
ACP206
ACP207
ACP208
ACP211
ACP213
ACP214
ACP215
ACP240
ACP241
ACP242
ACP243
ACP244
ACP245
ACP247
ACP284
ACP285
ACP286
ACP287
ACP288
ACP289
ACP290
ACP291
ACP292
ACP296
ACP297
ACP298
ACP299
ACP300
ACP302
ACP303
ACP304
ACP305
ACP306
ACP309
ACP310
ACP311
ACP312
ACP313
ACP314
ACP336
ACP337
ACP338
ACP339
ACP340
ACP341
ACP342
ACP343
ACP344
ACP345
ACP346
ACP347
ACP348
ACP349
ACP350
ACP351
ACP352
ACP353
ACP354
ACP355
ACP356
ACP357
ACP358
ACP359
ACP371
ACP372
ACP373
ACP374
ACP375
ACP376
ACP377
ACP378
ACP379
ACP383
ACP384
ACP385
ACP386
ACP387
ACP388
ACP389
ACP390
ACP391
ACP392
ACP393
ACP394
ACP395
ACP396
ACP397
ACP398
ACP399
ACP400
ACP401
ACP402
ACP403
ACP404
ACP405
ACP406
ACP407
ACP408
ACP409
ACP410
ACP411
ACP412
ACP413
ACP414
ACP415
ACP416
ACP417
ACP418
ACP419
ACP420
ACP421
ACP422
ACP423
ACP424
ACP425
ACP426
ACP427
ACP428
ACP429
ACP430
ACP431
ACP432
ACP433
ACP434
ACP439
ACP440
ACP441
ACP442
ACP443
ACP444
ACP445
ACP446
ACP447
ACP451
ACP452
ACP453
ACP454
ACP455
ACP456
ACP457
ACP458
ACP459
ACP460
ACP461
ACP462
ACP463
ACP464
ACP465
ACP466
ACP467
ACP468
ACP469
ACP470
ACP471

The invention features fusion proteins that are conditionally active variants of a cytokine of interest. In one aspect, the full-length polypeptides of the invention have reduced or minimal cytokine-receptor activating activity even though they contain a functional cytokine polypeptide. Upon activation, e.g., by cleavage of a linker that joins a blocking moiety, e.g. a steric blocking polypeptide, in sequence to the active cytokine, the cytokine, e.g., IL-2, IL-7, IL-12, IL-15, IL-18, IL-21, IL-23, IFNalpha, IFNbeta, IFNgamma, TNFalpha, lymphotoxin, TGF-beta1, TGFbeta2, TGFbeta3, GM-CSF, CXCL10, CCL19, CCL20, CCL21 or functional fragment or mutein of any of the foregoing, can bind its receptor and effect signaling. If desired, the full-length polypeptides can include a blocking polypeptide moiety that also provides additional advantageous properties. For example, the full-length polypeptide can contain a blocking polypeptide moiety that also extends the serum half-life and/or targets the full-length polypeptide to a desired site of cytokine activity. Alternatively, the full-length fusion polypeptides can contain a serum half-life extension element and/or targeting domain that are distinct from the blocking polypeptide moiety. Preferably, the fusion protein contains at least one element or domain capable of extending in vivo circulating half-life. Preferably, this element is removed enzymatically in the desired body location (e.g. protease cleavage in the tumor microenvironment), restoring pharmacokinetic properties to the payload molecule (e.g. IL2 or IFNa) substantially similar to the naturally occurring payload molecule. The fusion proteins may be targeted to a desired cell or tissue. As described herein targeting is accomplished through the action of a blocking polypeptide moiety that also binds to a desired target, or through a targeting domain. The domain that recognizes a target antigen on a preferred target (for example a tumor-specific antigen), may be attached to the cytokine via a cleavable or non-cleavable linker. If attached by a non-cleavable linker, the targeting domain may further aid in retaining the cytokine in the tumor, and it may be considered a retention domain. The targeting domain does not necessarily need to be directly linked to the payload molecule, and it may be linked directly to another element of the fusion protein. This is especially true if the targeting domain is attached via a cleavable linker.

In one aspect is provided a fusion polypeptide comprising a cytokine polypeptide, or functional fragment or mutein thereof, and a blocking moiety, e.g. a steric blocking domain. The blocking moiety is fused to the cytokine polypeptide, directly or through a linker, and can be separated from the cytokine polypeptide by cleavage (e.g, protease mediated cleavage) of the fusion polypeptide at or near the fusion site or linker or in the blocking moiety. For example, when the cytokine polypeptide is fused to a blocking moiety through a linker that contains a protease cleavage site, the cytokine polypeptide is released from the blocking moiety and can bind its receptor, upon protease mediated cleavage of the linker. The linker is designed to be cleaved at the site of desired cytokine activity, for example in the tumor microenvironment, avoiding off-target cytokine activity and reducing overall toxicity of cytokine therapy.

The blocking moiety can also function as a serum half-life extension element. In some embodiments, the fusion polypeptide further comprises a separate serum half-life extension element. In some embodiments, the fusion polypeptide further comprises a targeting domain. In various embodiments, the serum half-life extension element is a water-soluble polypeptide such as optionally branched or multi-armed polyethylene glycol (PEG), full length human serum albumin (HSA) or a fragment that preserves binding to FcRn, an Fc fragment, or a nanobody that binds to FcRn directly or to human serum albumin.

In addition to serum half-life extension elements, the pharmaceutical compositions described herein preferably comprise at least one, or more targeting domains that bind to one or more target antigens or one or more regions on a single target antigen. It is contemplated herein that a polypeptide construct of the invention is cleaved, for example, in a disease-specific microenvironment or in the blood of a subject at the protease cleavage site and that the targeting domain(s) will bind to a target antigen on a target cell. At least one target antigen is involved in and/or associated with a disease, disorder or condition. Exemplary target antigens include those associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease.

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, or fibrotic tissue cell.

Target antigens, in some cases, are expressed on the surface of a diseased cell or tissue, for example a tumor or a cancer cell. Target antigens for tumors include but are not limited to Fibroblast activation protein alpha (FAPa), Trophoblast glycoprotein (5T4), Tumor-associated calcium signal transducer 2 (Trop2), Fibronectin EDB (EDB-FN), fibronectin EIIIB domain, CGS-2, EpCAM, EGFR, HER-2, HER-3, c-Met, FOLR1, FAP, and CEA. Pharmaceutical compositions disclosed herein, also include proteins comprising two antigen binding domains that bind to two different target antigens known to be expressed on a diseased cell or tissue. Exemplary pairs of antigen binding domains include but are not limited to EGFR/CEA, EpCAM/CEA, and HER-2/HER-3.

In some embodiments, the targeting polypeptides independently comprise a scFv, a VH domain, a VL domain, a non-Ig domain, or a ligand that specifically binds to the target antigen. In some embodiments, the targeting polypeptides specifically bind to a cell surface molecule. In some embodiments, the targeting polypeptides specifically bind to a tumor antigen. In some embodiments, the targeting polypeptides specifically and independently bind to a tumor antigen selected from at least one of EpCAM, EGFR, HER-2, HER-3, cMet, CEA, and FOLR1. In some embodiments, the targeting polypeptides specifically and independently bind to two different antigens, wherein at least one of the antigens is a tumor antigen selected from EpCAM, EGFR, HER-2, HER-3, cMet, CEA, and FOLR1. In some embodiments, the targeting polypeptide serves as a retention domain and is attached to the cytokine via a non-cleavable linker.

As described herein, the cytokine blocking moiety can bind to the cytokine and thereby block activation of the cognate receptor of the cytokine.

This disclosure also related to nucleic acids, e.g., DNA, RNA, mRNA, that encode the conditionally active proteins described herein, as well as vectors and host cells that contain such nucleic acids.

This disclosure also relates to pharmaceutical compositions that contain a conditionally active protein, nucleic acid that encodes the conditionally active protein, and vectors and host cells that contain such nucleic acids. Typically, the pharmaceutical composition contains one or more physiologically acceptable carriers and/or excipients.

The disclosure also relates to therapeutic methods that include administering to a subject in need thereof an effective amount of a conditionally active protein, nucleic acid that encodes the conditionally active protein, vector or host cells that contain such a nucleic acid, and pharmaceutical compositions of any of the foregoing. Typically, the subject has, or is at risk of developing, a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease.

The disclosure also relates to the use of a conditionally active protein, nucleic acid that encodes the conditionally active protein, vector or host cells that contain such a nucleic acid, and pharmaceutical compositions of any of the foregoing, for treating a subject in need thereof. Typically the subject has, or is at risk of developing, a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease.

The disclosure also relates to the use of a conditionally active protein, nucleic acid that encodes the conditionally active protein, vector or host cells that contain such a nucleic acid for the manufacture of a medicament for treating a disease, such as a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease.

### BRIEF DESCRIPTION OF THE DRAWINGS

FIG. 1A is a schematic illustrating a protease-activated cytokine or chemokine that includes a blocking moiety. The blocking moiety may optionally function as a serum half-life extending domain. To the left of the arrow the drawing shows that a cytokine is connected to a blocking moiety via a protease-cleavable linker, thus blocking its ability to bind to its receptor. To the right of the arrow the drawing shows that in an inflammatory or tumor environment a protease cleaves at a protease-cleavage site on the linker, releasing the blocking moiety and allowing the cytokine to bind to its receptor.

FIG. 1B is a schematic illustrating a protease-activated cytokine or chemokine wherein HSA (blocking moiety) is directly bound to the cytokine or chemokine of interest, with a protease cleavage site between the HSA and a cytokine or chemokine of interest. To the left of the arrow the drawing shows that a cytokine is connected to a blocking moiety via a protease-cleavable linker, thus blocking its ability to bind to its receptor. To the right of the arrow the drawing shows that in an inflammatory or tumor environment, the protease cleaves at a protease-cleavage site on linker, releasing the blocking moiety and allowing the cytokine to bind to its receptor.

FIG. 1C is a schematic illustrating a protease-activated cytokine or chemokine wherein more than one HSA (blocking moiety) is bound directly to the molecule of interest. If desired, one or more of the HSA can be bonded to the cytokine or chemokine through a linker, such as a linker that contains a protease cleavage site. To the left of the arrow the drawing shows that a cytokine is connected to a blocking moiety via a protease-cleavable linker, thus blocking its ability to bind to its receptor. To the right of the arrow the drawing shows that in an inflammatory or tumor environment, protease cleaves at protease-cleavage site on linker, releasing the blocking moiety and allowing cytokine to bind receptor. The cytokine now has similar pK properties as compared to the native cytokine (e.g., has a short half-life).

FIG. 1D is a schematic illustrating a protease-activated cytokine or chemokine comprising more than one cytokine, of the same type or different type, each of which is bonded to a binding domain through a protease-cleavable linker. To the left of the arrow the drawing shows that a cytokine is connected to a blocking moiety via a protease-cleavable linker, thus blocking its ability to bind to its receptor. To the right of the arrow the drawing shows that in an inflammatory or tumor environment a protease cleaves at a protease cleavage site on linker, releasing the blocking moiety and allowing the cytokine to bind to its receptor.

FIG. 2 is a schematic illustrating a protease-activated cytokine or chemokine comprising a cytokine or chemokine polypeptide, a blocking moiety, and a serum half-life extending domain connected by at least one protease-cleavable linker. To the left of the arrow the drawing shows that a cytokine is connected to a blocking moiety via protease-cleavable linkers, thus blocking its ability to bind to its receptor. It is also bound to a separate half-life extension element, which extends half-life in serum. To the right of the arrow the drawing shows that in an inflammatory or tumor environment a protease cleaves at a protease-cleavage site on linker, thus releasing the serum half-life extension element and the blocking moiety and allowing the cytokine to bind to its receptor. The cytokine now has similar pK properties as compared to the native cytokine (e.g., a short half-life).

FIG. 3 is a schematic illustrating a protease-activated cytokine or chemokine comprising a cytokine or chemokine polypeptide, a blocking moiety, and a targeting domain connected by at least one protease-cleavable linker. To the left of the arrow the drawing shows that a cytokine is connected to a blocking moiety and a targeting domain via a protease-cleavable linker, thus blocking its ability to bind to its receptor. To the right of the arrow the drawing shows that in an inflammatory or tumor microenvironment a protease cleaves at the protease cleavage site in the linker, releasing the targeting domain and the blocking moiety and allowing the cytokine to bind to its receptor.

FIG. 4A is a schematic illustrating a protease-activated cytokine or chemokine comprising a cytokine or chemokine polypeptide, a blocking moiety, a targeting domain, and a serum half-life extending domain connected by at least one protease-cleavable linker, wherein the cytokine polypeptide and the targeting domain are connected by a protease-cleavable linker. To the left of the arrow, the drawing shows that a cytokine polypeptide is connected to targeting domain, blocking moiety, and half-life extension element via protease-cleavable linker(s), thus blocking its ability to bind to its receptor. To the right of the arrow the drawing shows that in an inflammatory or tumor environment, the protease cleaves at a protease-cleavage site on linker(s), releasing the half-life extension element, the targeting domain, and the blocking moiety, and allowing the cytokine to bind to its receptor. The cytokine now has similar pK properties as compared to the native cytokine (e.g., short half-life).

FIG. 4B is a schematic illustrating a protease-activated cytokine or chemokine comprising a cytokine or chemokine polypeptide, a blocking moiety, a targeting domain, and a serum half-life extending domain connected by at least one protease-cleavable linker. To the left of the arrow, the drawing shows that a cytokine is connected to targeting domain, a blocking moiety, and a half-life extension element via protease-cleavable linker(s), thus blocking its ability to bind to its receptor. To the right of the arrow the drawing shows that in an inflammatory or tumor environment, the protease cleaves at a protease-cleavage site on linker(s), releasing the half-life extension element and the blocking moiety and allowing the cytokine to bind to the receptor. The targeting moiety remains bound, keeping the cytokine in the tumor microenvironment. The cytokine now has similar pK properties as compared to the native cytokine (e.g., a short half-life).

FIG. 5 is a schematic illustrating the structure of a variable domain of an immunoglobulin molecule. The variable domains of both light and heavy immunoglobulin chains contain three hypervariable loops, or complementarity-determining regions (CDRs). The three CDRs of a V domain (CDR1, CDR2, CDR3) cluster at one end of the beta barrel. The CDRs are the loops that connect beta strands B-C, C′-C″, and F-G of the immunoglobulin fold, whereas the bottom loops that connect beta strands AB, CC′, C″-D and E-F of the immunoglobulin fold, and the top loop that connects the D-E strands of the immunoglobulin fold are the non-CDR loops.

FIG. 6. Place holder

FIGS. 7A-7H are a series of graphs showing activity of exemplary IL-2 fusion proteins in IL-2 dependent cytotoxic T lymphocyte cell line CTLL-2. Each graph shows results of the IL-2 proliferation assay as quantified by CellTiter-Glo® (Promega) luminescence-based cell viability assay. Each proliferation assay was performed with HSA (FIGS. 7B, 7D, 7F, 7H) or without (FIGS. 7A, 7C, 7E, 7G). Each fusion protein comprises an anti-HSA binder, and both uncleaved and MMP9 protease cleaved versions of the fusion protein were used in each assay.

FIGS. 8A-8F are a series of graphs showing activity of exemplary IL-2 fusion proteins in IL-2 dependent cytotoxic T lymphocyte cell line CTLL-2. Each graph shows results of the IL-2 proliferation assay as quantified by CellTiter-Glo (Promega) luminescence-based cell viability assay. Both uncleaved and MMP9 protease cleaved versions of the fusion protein were used in each assay.

FIGS. 9A-9Z are a series of graphs showing activity of exemplary IL-2 fusion proteins in IL-2 dependent cytotoxic T lymphocyte cell line CTLL-2. Each graph shows results of the IL-2 proliferation assay as quantified by CellTiter-Glo (Promega) luminescence-based cell viability assay. Both uncleaved and MMP9 protease cleaved versions of the fusion protein were used in each assay.

FIG. 10 shows results of protein cleavage assay. Fusion protein ACP16 was run on an SDS-PAGE gel in both cleaved and uncleaved form. As can be seen in the gel, cleavage was complete.

FIGS. 11A-11B are graphs depicting results from a HEK-Blue IL-12 reporter assay performed on human p40/murine p35 IL12 fusion proteins before and after protease cleavage. Constructs ACP35 (FIG. 11A) and ACP34 (FIG. 111B) were tested. Analysis was performed based on quantification of Secreted Alkaline Phosphatase (SEAP) activity using the reagent QUANTI-Blue® (InvivoGen). Results confirm that IL12 protein fusion proteins are active.

FIGS. 12A-12F show a series of graphs depicting the results of HEK-blue assay of four IL-12 fusion proteins, before and after cleavage by MMP9. Analysis was performed based on quantification of Secreted Alkaline Phosphatase (SEAP) activity using the reagent QUANTI-Blue (InvivoGen). The data show greater activity in the cleaved IL12 than in the full fusion protein. Constructs tested were ACP06 (FIG. 12A), ACP07 (FIG. 12C), ACP08 (FIG. 12B), ACP09 (FIG. 12D), ACP10 (FIG. 12E), ACP11 (FIG. 12F).

FIG. 13 shows results of protein cleavage assay. Fusion protein ACP11 was run on an SDS-PAGE gel in both cleaved and uncleaved form. As can be seen in the gel, cleavage was complete.

FIG. 14 is a schematic which depicts a non-limiting example of an inducible cytokine protein, wherein the construct is activated upon protease cleavage of a linker attached between two subunits of the cytokine.

FIGS. 15A-15D are graphs depicting results from a HEK-Blue assay performed on human p40/murine p35 IL12 fusion proteins before and after protease cleavage. Results confirm that IL12 protein fusion proteins are active. Each proliferation assay was performed with HSA or without HSA.

FIGS. 16A-16F are a series of graphs showing activity of exemplary IFNγ fusion proteins compared to activity of mouse IFNγ control using WEHI 279 cell survival assay. Each assay was performed with medium containing HSA (+HSA) or not containing HSA (-HSA). Each fusion protein comprises an anti-HSA binder, and both uncleaved and MMP9 protease cleaved versions of the fusion protein were used in each assay.

FIGS. 17A-17F are a series of graphs showing activity of exemplary IFNγ fusion proteins compared to activity of mouse IFNγ control using B16 reporter assay. Each assay was performed with medium containing HSA (+HSA) or not containing HSA (-HSA). Each fusion protein comprises an anti-HSA binder, and both uncleaved and MMP9 protease cleaved versions of the fusion protein were used in each assay.

FIGS. 18A-18B show results of protein cleavage assay, as described in Example 2. Two constructs, ACP31 (IFN-α fusion protein; FIG. 18A) and ACP55 (IFN-γ fusion protein; 18B), were run on an SDS-PAGE gel in both cleaved and uncleaved form. As can be seen in the gel, cleavage was complete.

FIGS. 19A-19B are a series of graphs (FIGS. 19A and 19B) showing activity of exemplary IFNγ fusion proteins before and after protease cleavage using B16 reporter assay. Each assay was performed with culture medium containing HSA, and each fusion protein comprises an anti-HSA binder. Both uncleaved and MMP9 protease cleaved versions of the fusion protein were used in each assay.

FIGS. 20A-20B are a series of graphs (FIG. 20A and FIG. 20B) showing activity of exemplary IFNa fusion proteins before and after cleavage using a B16 reporter assay. Each assay was performed with medium containing HSA, and each fusion protein comprises an anti-HSA binder. Both uncleaved and MMP9 protease cleaved versions of the fusion protein were used in each assay.

FIGS. 21A-21D are a series of graphs depicting the results of tumor growth studies using the MC38 cell line. FIGS. 21A-C show the effect of IFNγ and IFNγ fusion proteins on tumor growth when injected intraperitoneally (IP) using different dosing levels and schedules (ug=micrograms, BID=twice daily, BIW=twice weekly, QW=weekly). FIG. 21D shows the effect of intratumoral (IT) injection of IFNγ and IL-2 on tumor growth.

FIGS. 22A-22B are a series of graphs showing activity of exemplary IFNγ fusion proteins (ACP51 (FIG. 22A), and ACP52 (FIG. 22B)) cleaved by MMP9 protease compared to activity of uncleaved fusion proteins using B16 reporter assay. Each fusion protein comprises an anti-HSA binder and a tumor targeting domain.

FIGS. 23A-23B are a series of graphs showing activity of exemplary IFNγ fusion proteins (ACP53 and ACP54) cleaved by MMP9 protease compared to activity of uncleaved fusion proteins using B16 reporter assay. Each fusion protein comprises IFNγ directly fused to albumin.

FIGS. 24A-24D are graphs depicting results from a HEK-Blue IL-2 reporter assay performed on IL-2 fusion proteins and recombinant human IL2 (Rec hIL-2). Analysis was performed based on quantification of Secreted Alkaline Phosphatase (SEAP) activity using the reagent QUANTI-Blue (InvivoGen). FIG. 24A shows results of IL-2 constructs ACP132 and ACP 133 with and without albumin. FIG. 24B shows results of IL-2 construct ACP16 cleaved and uncleaved. Results of a protein cleavage assay of ACP16 in cleaved and uncleaved forms is also depicted. FIG. 24C shows results of IL-2 construct ACP153 in cleaved and uncleaved forms. Results of a protein cleavage assay are also depicted. FIG. 24D illustrates the results from a HEK-Blue IL-2 assay using wild-type cytokine, intact fusion protein, and protease-cleaved fusion protein.

FIGS. 25A and 25B are two graphs showing analysis of ACP16 (FIG. 25A) and ACP124 (FIG. 25B) in a HEKBlue IL-2 reporter assay in the presence of HSA. Circles depict the activity of the uncut polypeptide, squares depict activity of the cut polypeptide. FIG. 25C is a graph showing results of a CTLL-2 proliferation assay. CTLL2 cells (ATCC) were plated in suspension at a concentration of 500,000 cells/well in culture media with or without 40 mg/ml human serum albumin (HSA) and stimulated with a dilution series of activatable hIL2 for 72 hours at 37° C. and 5% CO₂. Activity of uncleaved and cleaved activatable ACP16 was tested. Cleaved activatable hIL2 was generated by incubation with active MMP9. Cell activity was assessed using a CellTiter-Glo (Promega) luminescence-based cell viability assay. Circles depict intact fusion protein, and squares depict protease-cleaved fusion protein.

FIGS. 26A-26C are a series of graphs showing activity of fusion proteins in an HEKBlue IL-12 reporter assay. FIG. 26A depicts IL-12/STAT4 activation in a comparison of ACP11 (a human p40/murine p35 IL12 fusion protein) to ACP04 (negative control). FIG. 26B is a graph showing analysis of ACP91 (a chimeric IL-12 fusion protein). Squares depict activity of the uncut ACP91 polypeptide, and triangles depict the activity of the cut polypeptide (ACP91+MMP9). EC50 values for each are shown in the table. FIG. 26C is a graph showing analysis of ACP136 (a chimeric IL-12 fusion protein). Squares depict activity of the uncut ACP136 polypeptide, and triangles depict the activity of the cut polypeptide (ACP136+MMP9). EC50 values for each are shown in the table insert.

FIGS. 27A-27F are a series of graphs showing that cleaved mouse IFNα1 polypeptides ACP31 (FIG. 27A), ACP125 (FIG. 27B), ACP126 (FIG. 27C) are active in an B16-Blue IFN-α/β reporter assay.

FIGS. 28A-28N are a series of graphs depicting the activity of ACP56 (FIG. 28A), ACP57 (FIG. 28B) ACP58 (FIG. 28C), ACP59 (FIG. 28D), ACP60 (FIG. 28E), ACP61+HSA (FIG. 28F), ACP30+HSA (FIG. 28G), ACP73 (FIG. 28H), ACP70+HSA (FIG. 281), ACP71 (FIG. 28J), ACP72 (FIG. 28K), ACP 73 (FIG. 28L), ACP74 (FIG. 28M), and ACP75 (FIG. 28N) in a B16 IFNγ reporter assay. Each fusion was tested for its activity when cut (squares) and uncut (circles).

FIGS. 29A-29B are two graphs showing results of analyzing ACP31 (mouse IFNα1 fusion protein) and ACP11 (a human p40/murine p35 IL12 fusion protein) in a tumor xenograft model. FIG. 29A shows tumor volume over time in mice treated with 33 μg ACP31 (circles), 110 μg ACP31 (triangles), 330 μg ACP31 (diamonds), and as controls 1 g murine wild type IFNα1 (dashed line, squares) and 10 μg mIFNα1 (dashed line, small circles). Vehicle alone is indicated by large open circles. The data show tumor volume decreasing over time in a dose-dependent manner in mice treated with ACP31. FIG. 29B shows tumor volume over time in mice treated with 17.5 μg ACP11 (squares), 175 μg ACP31 (triangles), 525 μg ACP31 (circles), and as controls 2 μg ACP04 (dashed line, triangles) and 10 μg ACP04 (dashed line, diamonds). Vehicle alone is indicated by large open circles. The data show tumor volume decreasing over time in a dose-dependent manner in mice treated with both ACP11 and ACP04 (a human p40/murine p35 IL12 fusion protein).

FIGS. 30A-30F are a series of spaghetti plots showing tumor volume over time in a mouse xenograft tumor model in mice each treated with vehicle alone (FIG. 30A), 2 μg ACP04 (FIG. 30B), 10 μg ACP04 (FIG. 30C, 17.5 g ACP11 (FIG. 30D), 175 μg ACP11 (FIG. 30E), and 525 μg ACP11 (FIG. 30F). Each line represents a single mouse.

FIG. 31A-31C are three graphs showing results of analyzing ACP16 and ACP124 in a tumor xenograft model. FIG. 31A shows tumor volume over time in mice treated with 4.4 μg ACP16 (squares), 17 μg ACP16 (triangles), 70 μg ACP16 (downward triangles), 232 μg ACP16 (dark circles), and as a comparator 12 g wild type IL-2 (dashed line, triangles) and 36 g wild type IL-2 (dashed line, diamonds. Vehicle alone is indicated by large open circles. The data show tumor volume decreasing over time in a dose-dependent manner in mice treated with ACP16 at higher concentrations. FIG. 31B shows tumor volume over time in mice treated with 17 μg ACP124 (squares), 70 μg ACP124 (triangles), 230 μg ACP124 (downward triangles), and 700 μg ACP124. Vehicle alone is indicated by large open circles. FIG. 31C shows tumor volume over time in mice treated with 17 μg ACP16 (triangles), 70 μg ACP16 (circles), 232 μg ACP16 (dark circles), and as a comparator 17 μg ACP124 (dashed line, triangles) 70 μg ACP124 (dashed line, diamonds), 230 μg ACP124 (dashed line, diamonds). Vehicle alone is indicated by dark downward triangles. The data show tumor volume decreasing over time in a dose-dependent manner in mice treated with ACP16, but not ACP124.

FIG. 32A Place holder

FIGS. 32B-32C are a series of spaghetti plots showing activity of fusion proteins in an MC38 mouse xenograft model corresponding to the data shown in FIG. 31. Each line in the plots is a single mouse.

FIG. 33 is a graph showing tumor volume over time in a mouse xenograft model showing tumor growth in control mice (open circles) and AP16-treated mice (squares).

FIGS. 34A-34D are a series of survival plots showing survival of mice over time after treatment with cleavable fusion proteins. FIG. 34A shows data for mice treated with vehicle alone (gray line), 17 μg ACP16 (dark line), and 17 μg ACP124 (dashed line). FIG. 34B shows data for mice treated with vehicle alone (gray line), 70 μg ACP16 (dark line), and 70 μg ACP124 (dashed line). FIG. 34C shows data for mice treated with vehicle alone (gray line), 232 μg ACP16 (dark line), and 230 μg ACP124 (dashed line). FIG. 34D shows data for mice treated with vehicle alone (gray line), 232 μg ACP16 (dark line), and 700 μg ACP124 (dashed line).

FIGS. 35A-35B a series of spaghetti plots showing activity of fusion proteins in an MC38 mouse xenograft model. All mouse groups were given four doses total except for the highest three doses of APC132, wherein fatal toxicity was detected after 1 week/2 doses. Shown are vehicle alone, 17, 55, 70, and 230 μg ACP16, 9, 28, 36, and 119 μg ACP132, and 13, 42, 54, and 177 μg ACP21. Each line in the plots represents an individual animal.

FIGS. 36-41 Place holder

FIGS. 42A-42E shows the results of B16 IFN reporter assays. Inducible interferon constructs of interest were tested before and after cleavage. The relevant wildtype IFN was tested as a control.

FIG. 43 shows binding data of ACP16, ACP10, ACP11

FIGS. 44A-44D depict the activity of cytokine fusion proteins constructs ACP243, ACP244, ACP243, ACP244, and ACP247.

FIGS. 45A-45B shows a series of spider plots showing tumor volume over time during treatment with vehicle, IL-12, ACP1 1 or ACP10.

FIGS. 46A-46D, 47A-47D, 48A-48B, 49A-49I, 50A-50B and 51A-51C shows data (tumor volume and/or body weight) for mice treated with cytokine fusion proteins constructs.

FIGS. 52A-52N, 53A, 53B depict the activity of cytokine fusion proteins constructs.

FIG. 54A-54N shows the results of proliferation assays comparing cut protein, uncut protein, and IL2 as a control.

FIGS. 55A-55N shows the results of HekBlue IL2 reporter assays comparing activity of constructs with and without protease cleavage; IL-2 is included as a control.

FIGS. 56. 57A-57D, 58, 59A-59C, 59E-59Z and 59AA depict the activity of cytokine fusion proteins constructs.

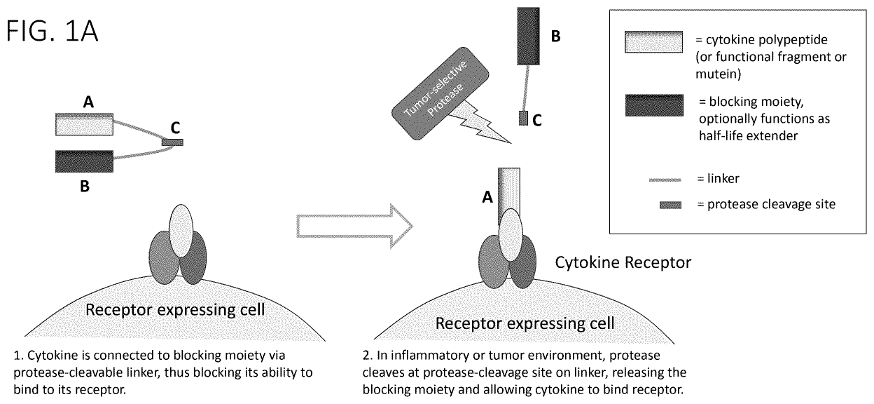

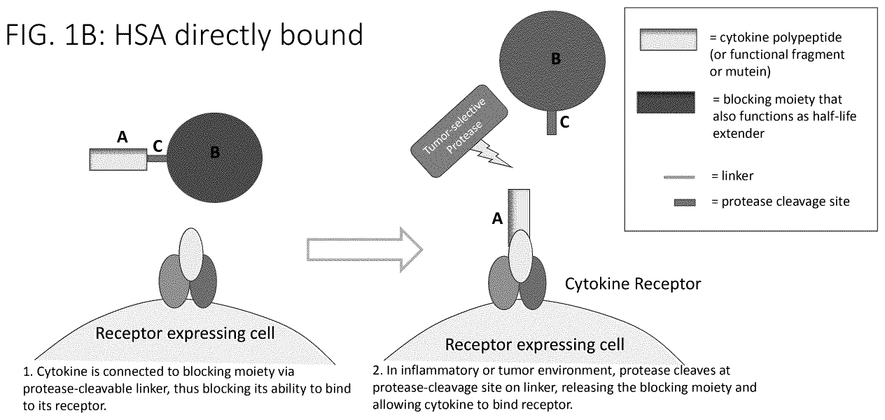

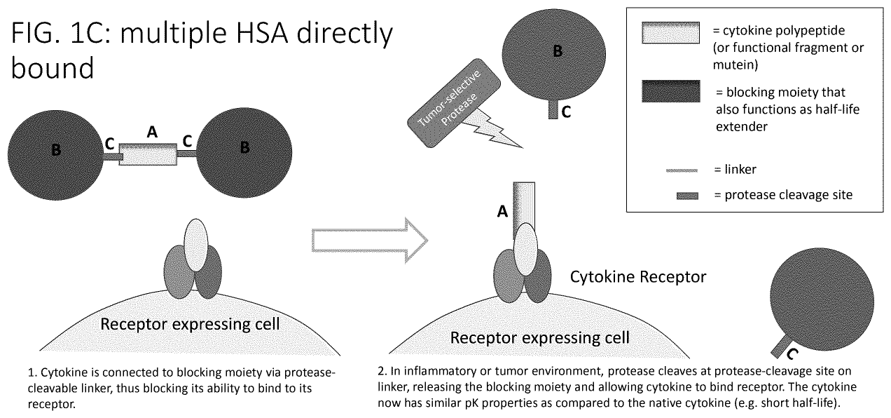

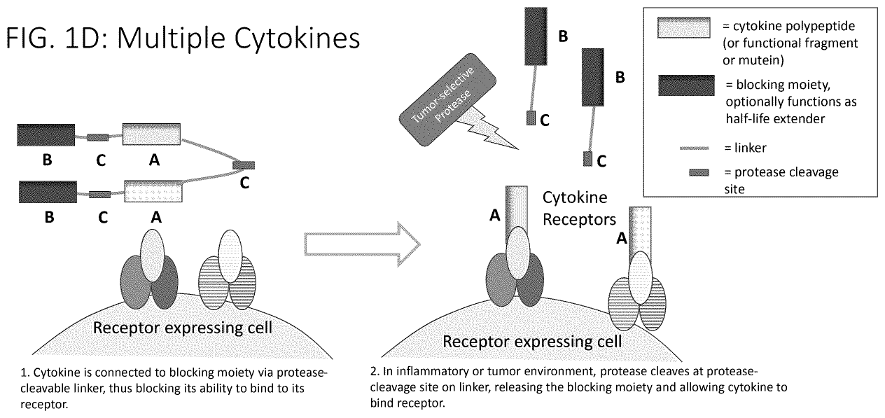

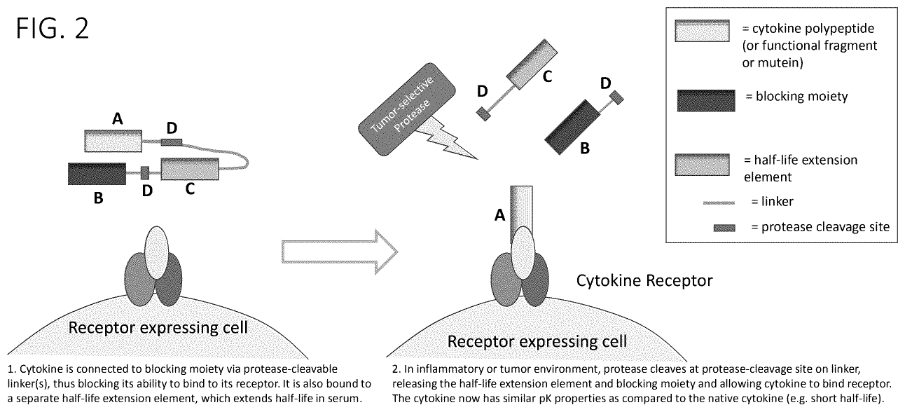

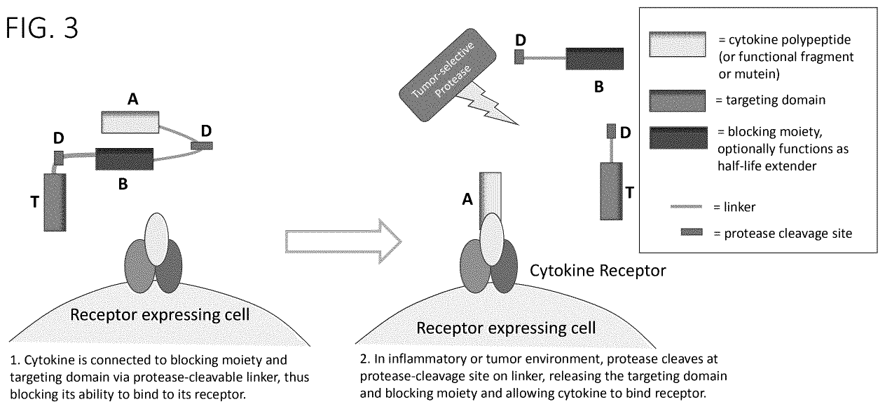

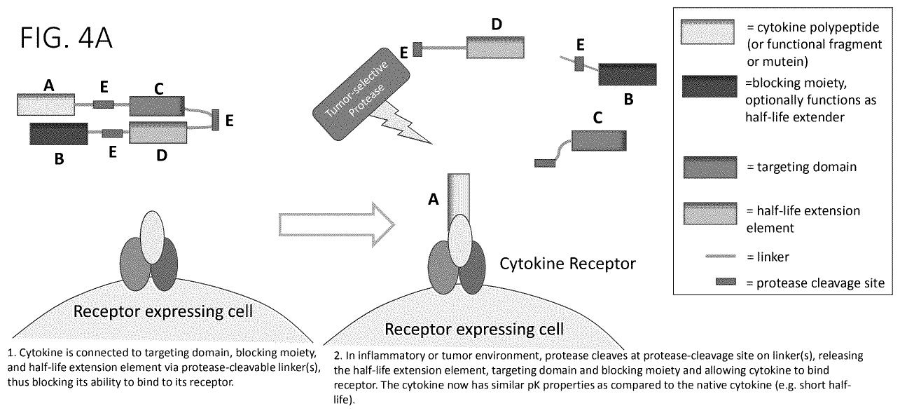

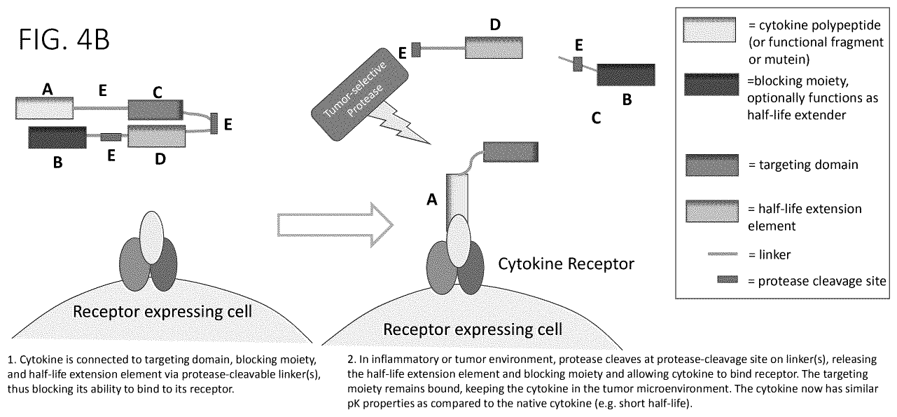

### DETAILED DESCRIPTION

Disclosed herein are methods and compositions to engineer and use constructs comprising inducible cytokines. Cytokines are potent immune agonists, which lead to them being considered promising therapeutic agents for oncology. However, cytokines proved to have a very narrow therapeutic window. Cytokines have short serum half-lives and are also considered to be highly potent. Consequently, therapeutic administration of cytokines produced undesirable systemic effects and toxicities. These were exacerbated by the need to administer large quantities of cytokine in order to achieve the desired levels of cytokine at the intended site of cytokine action (e.g., a tumor). Unfortunately, due to the biology of cytokines and inability to effectively target and control their activity, cytokines did not achieve the hoped-for clinical advantages in the treatment of tumors.

Disclosed herein are fusion proteins that overcome the toxicity and short half-life problems that have severely limited the clinical use of cytokines in oncology. The fusion proteins contain cytokine polypeptides that have receptor agonist activity. But in the context of the fusion protein, the cytokine receptor agonist activity is attenuated and the circulating half-life is extended. The fusion proteins include protease cleave sites, which are cleaved by proteases that are associated with a desired site of cytokine activity (e.g., a tumor), and are typically enriched or selectively present at the site of desired activity. Thus, the fusion proteins are preferentially (or selectively) and efficiently cleaved at the desired site of activity to limit cytokine activity substantially to the desired site of activity, such as the tumor microenvironment. Protease cleavage at the desired site of activity, such as in a tumor microenvironment, releases a form of the cytokine from the fusion protein that is much more active as a cytokine receptor agonist than the fusion protein (typically at least about 100× more active than the fusion protein). The form of the cytokine that is released upon cleavage of the fusion protein typically has a short half-life, which is often substantially similar to the half-life of the naturally occurring cytokine, further restricting cytokine activity to the tumor microenvironment. Even though the half-life of the fusion protein is extended, toxicity is dramatically reduced or eliminated because the circulating fusion protein is attenuated and active cytokine is targeted to the tumor microenvironment. The fusion proteins described herein, for the first time, enable the administration of an effective therapeutic dose of a cytokine to treat tumors with the activity of the cytokine substantially limited to the tumor microenvironment, and dramatically reduces or eliminates unwanted systemic effects and toxicity of the cytokine.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

“Cytokine” is a well-known term of art that refers to any of a class of immunoregulatory proteins (such as interleukin or interferon) that are secreted by cells especially of the immune system and that are modulators of the immune system. Cytokine polypeptides that can be used in the fusion proteins disclosed herein include, but are not limited to transforming growth factors, such as TGF-α and TGF-β (e.g., TGFbeta1, TGFbeta2, TGFbeta3); interferons, such as interferon-α, interferon-β, interferon-γ, interferon-kappa and interferon-omega; interleukins, such as IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21 and IL-25; tumor necrosis factors, such as tumor necrosis factor alpha and lymphotoxin; chemokines (e.g., C—X—C motif chemokine 10 (CXCL10), CCL19, CCL20, CCL21), and granulocyte macrophage-colony stimulating factor (GM-CS), as well as fragments of such polypeptides that active the cognate receptors for the cytokine (i.e., functional fragments of the foregoing). “Chemokine” is a term of art that refers to any of a family of small cytokines with the ability to induce directed chemotaxis in nearby responsive cells.

Cytokines are well-known to have short serum half-lives that frequently are only a few minutes or hours. Even forms of cytokines that have altered amino acid sequences intended to extend the serum half-life yet retain receptor agonist activity typically also have short serum half-lives. As used herein, a “short-half-life cytokine” refers to a cytokine that has a substantially brief half-life circulating in the serum of a subject, such as a serum half-life that is less than 10, less than 15, less than 30, less than 60, less than 90, less than 120, less than 240, or less than 480 minutes. As used herein, a short half-life cytokine includes cytokines which have not been modified in their sequence to achieve a longer than usual half-life in the body of a subject and polypeptides that have altered amino acid sequences intended to extend the serum half-life yet retain receptor agonist activity. This latter case is not meant to include the addition of heterologous protein domains, such as a bona fide half-life extension element, such as serum albumin.

“Sortases” are transpeptidases that modify proteins by recognizing and cleaving a carboxyl-terminal sorting signal embedded in or terminally attached to a target protein or peptide. Sortase A catalyzes the cleavage of the LPXTG motif (SEQ ID NO.: 442) (where X is any standard amino acid) between the Thr and Gly residue on the target protein, with transient attachment of the Thr residue to the active site Cys residue on the enzyme, forming an enzyme-thioacyl intermediate. To complete transpeptidation and create the peptide-monomer conjugate, a biomolecule with an N-terminal nucleophilic group, typically an oligoglycine motif, attacks the intermediate, displacing Sortase A and joining the two molecules.

As used herein, the term “steric blocker” refers to a polypeptide or polypeptide moiety that can be covalently bonded to a cytokine polypeptide directly or indirectly through other moieties such as linkers, for example in the form of a chimeric polypeptide (fusion protein), but otherwise does not covalently bond to the cytokine polypeptide. A steric blocker can non-covalently bond to the cytokine polypeptide, for example though electrostatic, hydrophobic, ionic or hydrogen bonding. A steric blocker typically inhibits or blocks the activity of the cytokine moiety due to its proximity to the cytokine moiety and comparative size. A steric blocker may also block by virtue of recruitment of a large protein binding partner. An example of this is an antibody which binds to serum albumin; while the antibody itself may or may not be large enough to block activation or binding on its own, recruitment of albumin allows for sufficient steric blocking.

As used and described herein, a “half-life extension element” is a part of the chimeric polypeptide that increases the serum half-life and improve pK, for example, by altering its size (e.g., to be above the kidney filtration cutoff), shape, hydrodynamic radius, charge, or parameters of absorption, biodistribution, metabolism, and elimination.

As used herein, the terms “activatable,” “activate,” “induce,” and “inducible” refer to the ability of a protein, i.e. a cytokine, that is part of a fusion protein, to bind its receptor and effectuate activity upon cleavage of additional elements from the fusion protein.

As used herein, “plasmids” or “viral vectors” are agents that transport the disclosed nucleic acids into the cell without degradation and include a promoter yielding expression of the nucleic acid molecule and/or polypeptide in the cells into which it is delivered.

As used herein, the terms “peptide”, “polypeptide”, or “protein” are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

As used throughout, “subject” can be a vertebrate, more specifically a mammal (e.g. a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered.

As used herein, “patient” or “subject” may be used interchangeably and can refer to a subject with a disease or disorder (e.g. cancer). The term patient or subject includes human and veterinary subjects.

As used herein the terms “treatment”, “treat”, or “treating” refers to a method of reducing the effects of a disease or condition or symptom of the disease or condition. Thus, in the disclosed method, treatment can refer to at least about 10%, at least about 20%, at least about 30%, at least about 40%, at least about 50%, at least about 60%, at least about 70%, at least about 80%, at least about 90%, or substantially complete reduction in the severity of an established disease or condition or symptom of the disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms “prevent”, “preventing”, and “prevention” of a disease or disorder refers to an action, for example, administration of the chimeric polypeptide or nucleic acid sequence encoding the chimeric polypeptide, that occurs before or at about the same time a subject begins to show one or more symptoms of the disease or disorder, which inhibits or delays onset or exacerbation of one or more symptoms of the disease or disorder.

As used herein, references to “decreasing”, “reducing”, or “inhibiting” include a change of at least about 10%, of at least about 20%, of at least about 30%, of at least about 40%, of at least about 50%, of at least about 60%, of at least about 70%, of at least about 80%, of at least about 90% or greater as compared to a suitable control level. Such terms can include but do not necessarily include complete elimination of a function or property, such as agonist activity.

An “attenuated cytokine receptor agonist” is a cytokine receptor agonist that has decreased receptor agonist activity as compared to the cytokine receptor's naturally occurring agonist. An attenuated cytokine agonist may have at least about 10×, at least about 50×, at least about 100×, at least about 250×, at least about 500×, at least about 1000× or less agonist activity as compared to the receptor's naturally occurring agonist. When a fusion protein that contains a cytokine polypeptide as described herein is described as “attenuated” or having “attenuated activity”, it is meant that the fusion protein is an attenuated cytokine receptor agonist.

An “intact fusion protein” is a fusion protein in which no domain has been removed, for example by protease cleavage. A domain may be removable by protease cleavage or other enzymatic activity, but when the fusion protein is “intact”, this has not occurred.

As used herein “moiety” refers to a portion of a molecule that has a distinct function within that molecule, and that function may be performed by that moiety in the context of another molecule. A moiety may be a chemical entity with a particular function, or a portion of a biological molecule with a particular function. For example, a “blocking moiety” within a fusion protein is a portion of the fusion protein which is capable of blocking the activity of some or all of the fusion polypeptide. This may be a protein domain, such as serum albumin. Blocking may be accomplished by a steric blocker or a specific blocker. A steric blocker blocks by virtue of size and position and not based upon specific binding; an examples is serum albumin. A specific blocker blocks by virtue of specific interactions with the moiety to be blocked. A specific blocker must be tailored to the particular cytokine or active domain; a steric blocker can be used regardless of the payload, as long as it is large enough.

In general, the therapeutic use of cytokines is strongly limited by their systemic toxicity. TNF, for example, was originally discovered for its capacity of inducing the hemorrhagic necrosis of some tumors, and for its in vitro cytotoxic effect on different tumoral lines, but it subsequently proved to have strong pro-inflammatory activity, which can, in case of overproduction conditions, dangerously affect the human body. As the systemic toxicity is a fundamental problem with the use of pharmacologically active amounts of cytokines in humans, novel derivatives and therapeutic strategies are now under evaluation, aimed at reducing the toxic effects of this class of biological effectors while keeping their therapeutic efficacy.

IL-2 exerts both stimulatory and regulatory functions in the immune system and is, along with other members of the common γ chain (γc) cytokine family, central to immune homeostasis. IL-2 mediates its action by binding to IL-2 receptors (IL-2R), consisting of either trimeric receptors made of IL-2Rα (CD25), IL-2Rβ (CD122), and IL-2Rγ (γc, CD132) chains or dimeric βγ IL-2Rs (1, 3). Both IL-2R variants are able to transmit signal upon IL-2 binding. However, trimeric αβγ IL-2Rs have a roughly 10-100 times higher affinity for IL-2 than dimeric βγ IL-2Rs (3), implicating that CD25 confers high-affinity binding of IL-2 to its receptor but is not crucial for signal transduction. Trimeric IL-2Rs are found on activated T cells and CD4+ forkhead box P3 (FoxP3)+ T regulatory cells (Treg), which are sensitive to IL-2 in vitro and in vivo. Conversely, antigen-experienced (memory) CD8+, CD44 high memory-phenotype (MP) CD8+, and natural killer (NK) cells are endowed with high levels of dimeric βγ IL-2Rs, and these cells also respond vigorously to IL-2 in vitro and in vivo.

Expression of the high-affinity IL-2R is critical for endowing T cells to respond to low concentrations of IL-2 that is transiently available in vivo. IL-2Rα expression is absent on naive and memory T cells but is induced after antigen activation. IL-2Rβ is constitutively expressed by NK, NKT, and memory CD8+ T cells but is also induced on naive T cells after antigen activation. γc is much less stringently regulated and is constitutively expressed by all lymphoid cells. Once the high-affinity IL-2R is induced by antigen, IL-2R signaling upregulates the expression of IL-2Rα in part through Stat5-dependent regulation of II2ra transcription (Kim et al., 2001). This process represents a mechanism to maintain expression of the high-affinity IL-2R and sustain IL-2 signaling while there remains a source of IL-2.

IL-2 is captured by IL-2Rα through a large hydrophobic binding surface surrounded by a polar periphery that results in a relatively weak interaction (Kd 10-8 M) with rapid on-off binding kinetics. However, the IL-2Rα-IL-2 binary complex leads to a very small conformational change in IL-2 that promotes association with IL-2Rβ through a distinct polar interaction between IL-2 and IL-2Rβ. The pseudo-high affinity of the IL2/α/β trimeric complex (i.e. Kd ~300 pM) clearly indicates that the trimeric complex is more stable than either IL2 bound to the a chain alone (Kd=10 nM) or to the β chain alone (Kd=450 nM) as shown by Ciardelli's data. In any event, the IL2/α/β trimer then recruits the γ chain into the quaternary complex capable of signaling, which is facilitated by the large composite binding site on the IL2-bound 3 chain for the γ chain.

In other words, the ternary IL-2Rα-IL-2Rβ-IL-2 complex then recruits γc through a weak interaction with IL-2 and a stronger interaction with IL-2Rβ to produce a stable quaternary high-affinity IL-2R (Kd 10-11 M which is 10 pM). The formation of the high-affinity quaternary IL-2-IL-2R complex leads to signal transduction through the tyrosine kinases Jak1 and Jak3, which are associated with IL-2Rβ and γc, respectively (Nelson and Willerford, 1998). The quaternary IL-2-IL-2R complex is rapidly internalized, where IL-2, IL-2Rβ, and γc are rapidly degraded, but IL-2Rα is recycled to the cell surface (Hémar et al., 1995; Yu and Malek, 2001). Thus, those functional activities that require sustained IL-2R signaling require a continued source of IL-2 to engage IL-2Rα and form additional IL-2-IL-2R signaling complexes.

Interleukin-15 (IL-15), another member of the 4-alpha-helix bundle family of cytokines, has also emerged as an immunomodulator for the treatment of cancer. IL-15 is initially captured via IL-15Ra, which is expressed on antigen-presenting dendritic cells, monocytes and macrophages. IL-15 exhibits broad activity and induces the differentiation and proliferation of T, B and natural killer (NK) cells via signaling through the IL-15/IL-2-R-β(CD122) and the common γ chain (CD132). It also enhances cytolytic activity of CD8+ T cells and induces long-lasting antigen-experienced CD8+CD44 memory T cells. IL-15 stimulates differentiation and immunoglobulin synthesis by B cells and induces maturation of dendritic cells. It does not stimulate immunosuppressive T regulatory cells (Tregs). Thus, boosting IL-15 activity selectively in the tumor micro-environment could enhance innate and specific immunity and fight tumors (Waldmann et al., 2012). IL-15 was initially identified for its ability to stimulate T cell proliferation in an IL-2-like manner through common receptor components (IL-2R/15Rβ-γc) and signaling through JAK1/JAK3 and STAT3/STAT5. Like IL-2, IL-15 has been shown to stimulate proliferation of activated CD4−CD8−, CD4+CD8+, CD4+ and CD8+ T cells as well as facilitate the induction of cytotoxic T-lymphocytes, and the generation, proliferation and activation of NK cells (Waldmann et al., 1999). However, unlike IL-2 which is required to maintain forkhead box P3 (FOXP3)-expressing CD4+CD25+ Treg cells and for the retention of these cells in the periphery, IL-15 has little effect on Tregs (Berger et al., 2009). This is important as FOXP3-expressing CD4+CD25+ Tregs inhibit effector T cells, thereby inhibiting immune responses including those directed against the tumor. IL-2 also has a crucial role in initiating activation induced cell death (AICD), a process that leads to the elimination of self-reactive T cells, whereas IL-15 is an anti-apoptotic factor for T cells (Marks-Konczalik et al., 2000). IL-15 co-delivered with HIV peptide vaccines has been shown to overcome CD4+ T cell deficiency by promoting longevity of antigen-specific CD8+ T cells and blocking TRAIL-mediated apoptosis (Oh et al., 2008). Furthermore, IL-15 promotes the long-term maintenance of CD8+CD44 hi memory T cells (Kanegane et al., 1996).

The importance of IL-15 and IL-15Rα to T and NK cell development is further highlighted by the phenotype of IL-15Ra^{-/-} and IL-15^{-/-} mice. Knockout mice demonstrate decreased numbers of total CD8+ T cells, and are deficient in memory-phenotype CD8+ T cells, NK cells, NK/T cells and some subsets of intestinal intraepithelial lymphocytes, indicating that IL-15 provides essential positive homeostatic functions for these subsets of cells (Lodolce et al., 1996; Kennedy et al., 1998). The similarities in the phenotypes of these two strains of knockout mice suggest the importance of IL-15Rα in maintaining physiologically relevant IL-15 signals.

IL-15 is presented in trans by the IL-15 receptor alpha-chain to the IL-15Rβτyc complex displayed on the surface of T cells and natural killer (NK) cells (Han et al., 2011). The IL-15Ra-chain plays a role of chaperone protein, stabilizes, and increases IL-15 activity (Desbois et al., 2016). It has been shown that exogenous IL-15 may have a limited impact on patients with cancer due to its dependency on IL-15Ra frequently downregulated in cancer patients. Therefore, the fusion protein RLI, composed of the sushi+domain of IL15Ra coupled via a linker to IL-15, has been suggested as an alternative approach to IL15 therapy (Bessard et al., 2009). It was found that administration of soluble IL-15/IL-15Rα complexes greatly enhanced IL-15 serum half-life and bioavailability in vivo (Stoklasek et al., 2010).

In addition to the effects on T and NK cells, IL-15 also has several effects on other components of the immune system. IL-15 protects neutrophils from apoptosis, modulates phagocytosis and stimulates the secretion of IL-8 and IL-1R antagonist. It functions through the activation of JAK2, p38 and ERK1/2 MAPK, Syk kinase and the NF-kB transcriptional factor (Pelletier et al., 2002). In mast cells, IL-15 can act as a growth factor and an inhibitor of apoptosis. In these cells IL-15 activates the JAK2/STAT5 pathway without the requirement of γc binding (Tagaya et al., 1996). IL-15 also induces B lymphocyte proliferation and differentiation, and increases immunoglobulin secretion (Armitage et al., 1995). It also prevents Fas-mediated apoptosis and allows induction of antibody responses partially independent of CD4-help (Demerci et al., 2004; Steel et al., 2010). Monocytes, macrophages and dendritic cells effectively transcribe and translate IL-15. They also respond to IL-15 stimulation. Macrophages respond by increasing phagocytosis, inducing IL-8, IL-12 and MCP-1 expression, and secreting IL-6, IL-8 and TNF α (Budagian et al., 2006). Dendritic cells incubated with IL-15 demonstrate maturation with increased CD83, CD86, CD40, and MHC class II expression, are also resistant to apoptosis, and show enhanced interferon-γ secretion (Anguille et al., 2009).

IL-15 has also been shown to have effects on non-hematological cells including myocytes, adipocytes, endothelial and neural cells. IL-15 has an anabolic effect on muscle and may support muscle cell differentiation (Quinn et al., 1995). It stimulates myocytes and muscle fibers to accumulate contractile protein and is able to slow muscle wasting in rats with cancer-related cachexia (Figueras et al., 2004). IL-15 has also been shown to stimulate angiogenesis (Angiolillo et al., 1997) and induce microglial growth and survival (Hanisch et al., 1997).

Interleukin-7 (IL-7), also of the IL-2/IL-15 family, is a well-characterized pleiotropic cytokine, and is expressed by stromal cells, epithelial cells, endothelial cells, fibroblasts, smooth muscle cells and keratinocytes, and following activation, by dendritic cells (Alpdogan et al., 2005). Although it was originally described as a growth and differentiation factor for precursor B lymphocytes, subsequent studies have shown that IL-7 is critically involved in T-lymphocyte development and differentiation. Interleukin-7 signaling is essential for optimal CD8 T-cell function, homeostasis and establishment of memory (Schluns et al., 2000); it is required for the survival of most T-cell subsets, and its expression has been proposed to be important for regulating T-cell numbers.

IL-7 binds to a dimeric receptor, including IL-7Rα and γ_{c} to form a ternary complex that plays fundamental roles in extracellular matrix remodeling, development, and homeostasis of T and B cells (Mazzucchelli and Durum, 2007). IL-7Rα also cross-reacts to form a ternary complex with thymic stromal lymphopoietin (TSLP) and its receptor (TSLPR), and activates the TSLP pathway, resulting in T and dendritic cell proliferation in humans and further B cell development in mice (Leonard, 2002). Tight regulation of the signaling cascades activated by the complexes are therefore crucial to normal cellular function. Under-stimulation of the IL-7 pathway caused by mutations in the IL-7Rα ectodomain inhibits T and B cell development, resulting in patients with a form of severe combined immunodeficiency (SCID) (Giliani et al., 2005; Puel et al., 1998).

IL-7 has a potential role in enhancing immune reconstitution in cancer patients following cytotoxic chemotherapy. IL-7 therapy enhances immune reconstitution and can augment even limited thymic function by facilitating peripheral expansion of even small numbers of recent thymic emigrants. Therefore, IL-7 therapy could potentially repair the immune system of patients who have been depleted by cytotoxic chemotherapy (Capitini et al., 2010).

Interleukin-12 (IL-12) is a disulfide-linked heterodimer of two separately encoded subunits (p35 and p40), which are linked covalently to give rise to the so-called bioactive heterodimeric (p70) molecule (Lieschke et al., 1997; Jana et al., 2014). Apart from forming heterodimers (IL-12 and IL-23), the p40 subunit is also secreted as a monomer (p40) and a homodimer (p40₂). It is known in the art that synthesis of the heterodimer as a single chain with a linker connecting the p35 to the p40 subunit preserves the full biological activity of the heterodimer. IL-12 plays a critical role in the early inflammatory response to infection and in the generation of Th1 cells, which favor cell-mediated immunity. It has been found that overproduction of IL-12 can be dangerous to the host because it is involved in the pathogenesis of a number of autoimmune inflammatory diseases (e.g. MS, arthritis, type 1 diabetes).

The IL-12 receptor (IL-12R) is a heterodimeric complex consisting of IL-12Rβ1 and IL-12Rβ2 chains expressed on the surface of activated T-cells and natural killer cells (Trinchieri et al., 2003). The IL-12Rβ1 chain binds to the IL-12p40 subunit, whereas IL-12p35 in association with IL-12Rβ2 confers an intracellular signaling ability (Benson et al., 2011). Signal transduction through IL-12R induces phosphorylation of Janus kinase (Jak2) and tyrosine kinase (Tyk2), that phosphorylate and activate signal transducer and activator of transcription (STAT)1, STAT3, STAT4, and STAT5. The specific cellular effects of IL-12 are due mainly to activation of STAT4. IL-12 induces natural killer and T-cells to produce cytokines, in particular interferon (IFN)γ, that mediate many of the proinflammatory activities of IL-12, including CD4+ T-cell differentiation toward the Th1 phenotype (Montepaone et al., 2014).

Regulatory T cells actively suppress activation of the immune system and prevent pathological self-reactivity and consequent autoimmune disease. Developing drugs and methods to selectively activate regulatory T cells for the treatment of autoimmune disease is the subject of intense research and, until the development of the present invention, which can selectively deliver active interleukins at the site of inflammation, has been largely unsuccessful. Regulatory T cells (Treg) are a class of CD4+CD25+ T cells that suppress the activity of other immune cells. Treg are central to immune system homeostasis, and play a major role in maintaining tolerance to self-antigens and in modulating the immune response to foreign antigens. Multiple autoimmune and inflammatory diseases, including Type 1 Diabetes (T1D), Systemic Lupus Erythematosus (SLE), and Graft-versus-Host Disease (GVHD) have been shown to have a deficiency of Treg cell numbers or Treg function.

Consequently, there is great interest in the development of therapies that boost the numbers and/or function of Treg cells. One treatment approach for autoimmune diseases being investigated is the transplantation of autologous, ex vivo-expanded Treg cells (Tang, Q., et al, 2013, Cold Spring Harb. Perspect. Med., 3:1-15). While this approach has shown promise in treating animal models of disease and in several early stage human clinical trials, it requires personalized treatment with the patient's own T cells, is invasive, and is technically complex. Another approach is treatment with low dose Interleukin-2 (IL-2). Treg cells characteristically express high constitutive levels of the high affinity IL-2 receptor, IL2Rαβγ, which is composed of the subunits IL2Rα (CD25), IL2Rβ (CD122), and IL2Rγ (CD132), and Treg cell growth has been shown to be dependent on IL-2 (Malek, T. R., et al., 2010, Immunity, 33:153-65).

Conversely, immune activation has also been achieved using IL-2, and recombinant IL-2 (Proleukin®) has been approved to treat certain cancers. High-dose IL-2 is used for the treatment of patients with metastatic melanoma and metastatic renal cell carcinoma with a long-term impact on overall survival.

Clinical trials of low-dose IL-2 treatment of chronic GVHD (Koreth, J., et al., 2011, N Engl J Med., 365:2055-66) and HCV-associated autoimmune vasculitis patients (Saadoun, D., et al., 2011, N Engl J Med., 365:2067-77) have demonstrated increased Treg levels and signs of clinical efficacy. New clinical trials investigating the efficacy of IL-2 in multiple other autoimmune and inflammatory diseases have been initiated. The rationale for using so-called low dose IL-2 was to exploit the high IL-2 affinity of the trimeric IL-2 receptor which is constitutively expressed on Tregs while leaving other T cells which do not express the high affinity receptor in the inactivated state. Aldesleukin (marketed as Proleukin® by Prometheus Laboratories, San Diego, CA), the recombinant form of IL-2 used in these trials, is associated with high toxicity. Aldesleukin, at high doses, is approved for the treatment of metastatic melanoma and metastatic renal cancer, but its side effects are so severe that its use is only recommended in a hospital setting with access to intensive care (Web address: www.proleukin.com/assets/pdf/proleukin.pdf).

The clinical trials of IL-2 in autoimmune diseases have employed lower doses of IL-2 in order to target Treg cells, because Treg cells respond to lower concentrations of IL-2 than many other immune cell types due to their expression of IL2R alpha (Klatzmann D, 2015 Nat Rev Immunol. 15:283-94). However, even these lower doses resulted in safety and tolerability issues, and the treatments used have employed daily subcutaneous injections, either chronically or in intermittent 5-day treatment courses. Therefore, there is a need for an autoimmune disease therapy that potentiates Treg cell numbers and function, that targets Treg cells more specifically than IL-2, that is safer and more tolerable, and that is administered less frequently.

One approach that has been suggested for improving the therapeutic index of IL-2-based therapy for autoimmune diseases is to use variants of IL-2 that are selective for Treg cells relative to other immune cells. IL-2 receptors are expressed on a variety of different immune cell types, including T cells, NK cells, eosinophils, and monocytes, and this broad expression pattern likely contributes to its pleiotropic effect on the immune system and high systemic toxicity. In particular, activated T effector cells express IL2Rαβγ, as do pulmonary epithelial cells. But, activating T effector cells runs directly counter to the goal of down-modulating and controlling an immune response, and activating pulmonary epithelial cells leads to known dose-limiting side effects of IL-2 including pulmonary edema. In fact, the major side effect of high-dose IL-2 immunotherapy is vascular leak syndrome (VLS), which leads to accumulation of intravascular fluid in organs such as lungs and liver with subsequent pulmonary edema and liver cell damage. There is no treatment of VLS other than withdrawal of IL-2. Low-dose IL-2 regimens have been tested in patients to avoid VLS, however, at the expense of suboptimal therapeutic results.

According to the literature, VLS is believed to be caused by the release of proinflammatory cytokines from IL-2-activated NK cells. However, there is some evidence that pulmonary edema results from direct binding of IL-2 to lung endothelial cells, which expressed low to intermediate levels of functional αβγ IL-2Rs. And, the pulmonary edema associated with interaction of IL-2 with lung endothelial cells was abrogated by blocking binding to CD25 with an anti-CD25 monoclonal antibody (mAb), in CD25-deficient host mice, or by the use of CD122-specific IL-2/anti-IL-2 mAb (IL-2/mAb) complexes, thus preventing VLS.

Treatment with interleukin cytokines other than IL-2 has been more limited. IL-15 displays immune cell stimulatory activity similar to that of IL-2 but without the same inhibitory effects, thus making it a promising immunotherapeutic candidate. Clinical trials of recombinant human IL-15 for the treatment of metastatic malignant melanoma or renal cell cancer demonstrated appreciable changes in immune cell distribution, proliferation, and activation and suggested potential antitumor activity (Conlon et. al., 2014). IL-15 is currently in clinical trials to treat various forms of cancer. However, IL-15 therapy is known to be associated with undesired and toxic effects, such as exacerbating certain leukemias, graft-versus-host disease, hypotension, thrombocytopenia, and liver injury. (Mishra A., et al., Cance Cell, 2012, 22(5):645-55; Alpdogan O. et al., Blood, 2005, 105(2):866-73; Conlon K C et al., J Clin Oncol, 2015, 33(1):74-82.)

IL-7 promotes lymphocyte development in the thymus and maintains survival of naive and memory T cell homeostasis in the periphery. Moreover, it is important for the organogenesis of lymph nodes (LN) and for the maintenance of activated T cells recruited into the secondary lymphoid organs (SLOs) (Gao et. al., 2015). In clinical trials of IL-7, patients receiving IL-7 showed increases in both CD4+ and CD8+ T cells, with no significant increase in regulatory T cell numbers as monitored by FoxP3 expression (Sportes et al., 2008). In clinical trials reported in 2006, 2008 and 2010, patients with different kinds of cancers such as metastatic melanoma or sarcoma were injected subcutaneously with different doses of IL-7. Little toxicity was seen except for transient fevers and mild erythema. Circulating levels of both CD4+ and CD8+ T cells increased significantly and the number of Treg reduced. TCR repertoire diversity increased after IL-7 therapy. However, the anti-tumor activity of IL-7 was not well evaluated (Gao et. al., 2015). Results suggest that IL-7 therapy could enhance and broaden immune responses.

IL-12 is a pleiotropic cytokine, the actions of which create an interconnection between the innate and adaptive immunity. IL-12 was first described as a factor secreted from PMA-induced EBV-transformed B-cell lines. Based on its actions, IL-12 has been designated as cytotoxic lymphocyte maturation factor and natural killer cell stimulatory factor. Due to bridging the innate and adaptive immunity and potently stimulating the production of IFNγ, a cytokine coordinating natural mechanisms of anticancer defense, IL-12 seemed ideal candidate for tumor immunotherapy in humans. However, severe side effects associated with systemic administration of IL-12 in clinical investigations and the very narrow therapeutic index of this cytokine markedly tempered enthusiasm for the use of this cytokine in cancer patients (Lasek et. al., 2014). Approaches to IL-12 therapy in which delivery of the cytokine is tumor-targeted, which may diminish some of the previous issues with IL-12 therapy, are currently in clinical trials for cancers.

The direct use of IL-2 as an agonist to bind the IL-2R and modulate immune responses therapeutically has been problematic due its well-documented therapeutic risks, e.g., its short serum half-life and high toxicity. These risks have also limited the therapeutic development and use of other cytokines. New forms of cytokines that reduce these risks are needed. Disclosed herein are compositions and methods comprising IL-2 and IL-15 and other cytokines, functional fragments and muteins of cytokines as well as conditionally active cytokines designed to address these risks and provide needed immunomodulatory therapeutics.

The present invention is designed to address the shortcomings of direct IL-2 therapy and therapy using other cytokines, for example using cytokine blocking moieties, e.g. steric blocking polypeptides, serum half-life extending polypeptides, targeting polypeptides, linking polypeptides, including protease cleavable linkers, and combinations thereof. Cytokines, including interleukins (e.g., IL-2, IL-7, IL-12, IL-15, IL-18, IL-21 IL-23), interferons (IFNs, including IFNalpha, IFNbeta and IFNgamma), tumor necrosis factors (e.g., TNFalpha, lymphotoxin), transforming growth factors (e.g., TGFbeta1, TGFbeta2, TGFbeta3), chemokines (C—X—C motif chemokine 10 (CXCL10), CCL19, CCL20, CCL21), and granulocyte macrophage-colony stimulating factor (GM-CS) are highly potent when administered to patients. As used herein, “chemokine” means a family of small cytokines with the ability to induce directed chemotaxis in nearby responsive cells Cytokines can provide powerful therapy, but are accompanied by undesired effects that are difficult to control clinically and which have limited the clinical use of cytokines. This disclosure relates to new forms of cytokines that can be used in patients with reduced or eliminated undesired effects. In particular, this disclosure relates to pharmaceutical compositions including chimeric polypeptides (fusion proteins), nucleic acids encoding fusion proteins and pharmaceutical formulations of the foregoing that contain cytokines or active fragments or muteins of cytokines that have decreased cytokine receptor activating activity in comparison to the corresponding cytokine. However, under selected conditions or in a selected biological environment the chimeric polypeptides activate their cognate receptors, often with the same or higher potency as the corresponding naturally occurring cytokine. As described herein, this is typically achieved using a cytokine blocking moiety that blocks or inhibits the receptor activating function of the cytokine, active fragment or mutein thereof under general conditions but not under selected conditions, such as those present at the desired site of cytokine activity (e.g., an inflammatory site or a tumor).

The chimeric polypeptides and nucleic acids encoding the chimeric polypeptides can be made using any suitable method. For example, nucleic acids encoding a chimeric polypeptide can be made using recombinant DNA techniques, synthetic chemistry or combinations of these techniques, and expressed in a suitable expression system, such as in CHO cells. Chimeric polypeptides can similarly be made, for example by expression of a suitable nucleic acid, using synthetic or semi-synthetic chemical techniques, and the like. In some embodiments, the blocking moiety can be attached to the cytokine polypeptide via sortase-mediated conjugation. “Sortases” are transpeptidases that modify proteins by recognizing and cleaving a carboxyl-terminal sorting signal embedded in or terminally attached to a target protein or peptide. Sortase A catalyzes the cleavage of the LPXTG motif (SEQ ID No.: 442) (where X is any standard amino acid) between the Thr and Gly residue on the target protein, with transient attachment of the Thr residue to the active site Cys residue on the enzyme, forming an enzyme-thioacyl intermediate. To complete transpeptidation and create the peptide-monomer conjugate, a biomolecule with an N-terminal nucleophilic group, typically an oligoglycine motif, attacks the intermediate, displacing Sortase A and joining the two molecules.

To form the cytokine-blocking moiety fusion protein, the cytokine polypeptide is first tagged at the N-terminus with a polyglycine sequence, or alternatively, with at the C-terminus with a LPXTG motif (SEQ ID NO.: 442). The blocking moiety or other element has respective peptides attached that serve as acceptor sites for the tagged polypeptides. For conjugation to domains carrying a LPXTG (SEQ ID NO.: 442) acceptor peptide attached via its N-terminus, the polypeptide will be tagged with an N-terminal poly-glycine stretch. For conjugation to domain carrying a poly-glycine peptide attached via its C-terminus, the polypeptide will be tagged at its C-terminus with a LPXTG (SEQ ID NO.: 442) sortase recognition sequence. Recognizing poly-glycine and LPXTG (SEQ ID NO.: 442) sequences, sortase will form a peptide bond between polymer-peptide and tagged polypeptides. The sortase reaction cleaves off glycine residues as intermediates and occurs at room temperature.

A variety of mechanisms can be exploited to remove or reduce the inhibition caused by the blocking moiety. For example, the pharmaceutical compositions can include a cytokine moiety and a blocking moiety, e.g. a steric blocking moiety, with a protease cleavable linker comprising a protease cleavage site located between the cytokine and cytokine blocking moiety or within the cytokine blocking moiety. When the protease cleavage site is cleaved, the blocking moiety can dissociate from cytokine, and the cytokine can then activate cytokine receptor. A cytokine moiety can also be blocked by a specific blocking moiety, such as an antibody, which binds an epitope found on the relevant cytokine.

Any suitable linker can be used. For example, the linker can comprise glycine-glycine, a sortase-recognition motif, or a sortase-recognition motif and a peptide sequence (Gly₄Ser)ₙ (SEQ ID NO.: 443) or (Gly₃Ser)ₙ, (SEQ ID NO.: 444) wherein n is 1, 2, 3, 4 or 5. Typically, the sortase-recognition motif comprises a peptide sequence LPXTG (SEQ ID NO.: 442), where X is any amino acid. In some embodiments, the covalent linkage is between a reactive lysine residue attached to the C-terminal of the cytokine polypeptide and a reactive aspartic acid attached to the N-terminal of the blocker or other domain. In other embodiments, the covalent linkage is between a reactive aspartic acid residue attached to the N-terminal of the cytokine polypeptide and a reactive lysine residue attached to the C-terminal of said blocker or other domain.

Accordingly, as described in detail herein, the cytokine blocking moieties used can be steric blockers. As used herein, a “steric blocker” refers to a polypeptide or polypeptide moiety that can be covalently bonded to a cytokine polypeptide directly or indirectly through other moieties such as linkers, for example in the form of a chimeric polypeptide (fusion protein), but otherwise does not covalently bond to the cytokine polypeptide. A steric blocker can non-covalently bond to the cytokine polypeptide, for example though electrostatic, hydrophobic, ionic or hydrogen bonding. A steric blocker typically inhibits or blocks the activity of the cytokine moiety due to its proximity to the cytokine moiety and comparative size. The steric inhibition of the cytokine moiety can be removed by spatially separating the cytokine moiety from the steric blocker, such as by enzymatically cleaving a fusion protein that contains a steric blocker and a cytokine polypeptide at a site between the steric blocker and the cytokine polypeptide.

As described in greater detail herein, the blocking function can be combined with or due to the presence of additional functional components in the pharmaceutical composition, such as a targeting domain, a serum half-life extension element, and protease-cleavable linking polypeptides. For example, a serum half-life extending polypeptide can also be a steric blocker.

In the interest of presenting a concise disclosure of the full scope of the invention, aspects of the invention are described in detail using the cytokine IL-2 as an exemplary cytokine. However, the invention and this disclosure are not limited to IL-2. It will be clear to a person of skill in the art that this disclosure, including the disclosed methods, polypeptides and nucleic acids, adequately describes and enables the use of other cytokines, fragments and muteins, such as IL-2, IL-7, IL-12, IL-15, IL-18, IL-21 IL-23, IFNalpha, IFNbeta, IFNgamma, TNFalpha, lymphotoxin, TGF-beta1, TGFbeta2, TGFbeta3, GM-CSF, CXCL10, CCL19, CCL20, CCL21 and functional fragments or muteins of any of the foregoing.

Various elements ensure the delivery and activity of IL-2 preferentially at the site of desired IL-2 activity and to severely limit systemic exposure to the interleukin via a blocking and/or a targeting strategy preferentially linked to a serum half-life extension strategy. In this serum half-life extension strategy, the blocked version of interleukin circulates for extended times (preferentially 1-2 or more weeks) but the activated version has the typical serum half-life of the interleukin.

By comparison to a serum half-life extended version, the serum half-life of IL-2 administered intravenously is only ~10 minutes due to distribution into the total body extracellular space, which is large, ~15 L in an average sized adult. Subsequently, IL-2 is metabolized by the kidneys with a half-life of ~2.5 hours. (Smith, K. “Interleukin 2 immunotherapy.” Therapeutic Immunology 240 (2001)). By other measurements, IL-2 has a very short plasma half-life of 85 minutes for intravenous administration and 3.3 hours subcutaneous administration (Kirchner, G. I., et al., 1998, Br J Clin Pharmacol. 46:5-10). In some embodiments of this invention, the half-life extension element is linked to the interleukin via a linker which is cleaved at the site of action (e.g. by inflammation-specific or tumor-specific proteases) releasing the interleukin's full activity at the desired site and also separating it from the half-life extension of the uncleaved version. In such embodiments, the fully active and free interleukin would have very different pharmacokinetic (pK) properties—a half-life of hours instead of weeks. In addition, exposure to active cytokine is limited to the site of desired cytokine activity (e.g., an inflammatory site or tumor) and systemic exposure to active cytokine, and associated toxicity and side effects, are reduced.

Other cytokines envisioned in this invention have similar pharmacology (e.g. IL-15 as reported by Blood 2011 117:4787-4795; doi: doi.org/10.1182/blood-2010-10-311456) as IL-2 and accordingly, the designs of this invention address the shortcomings of using these agents directly, and provide chimeric polypeptides that can have extended half-life and/or be targeted to a site of desired activity (e.g., a site of inflammation or a tumor).

If desired, IL-2 can be engineered to bind the IL-2R complex generally or one of the three IL-2R subunits specifically with an affinity that differs from that of the corresponding wild-type IL-2, for example toto selectively activate Tregs or Teff. For example, IL-2 polypeptides that are said to have higher affinity for the trimeric form of the IL-2 receptor relative to the dimeric beta/gamma form of the I1-2 receptor in comparison to wild type IL-2 can have an amino acid sequence that includes one of the following sets of mutations with respect to SEQ ID NO:1 (a mature IL-2 protein comprising amino acids 21-153 of human IL-2 having the Uniprot Accession No. P60568-1): (a) K64R, V69A, and Q74P; (b) V69A, Q74P, and T101A; (c) V69A, Q74P, and 1128T; (d) N30D, V69A, Q74P, and F103S; (e) K49E, V69A, A73V, and K76E; (f) V69A, Q74P, T101A, and T133N; (g) N30S, V69A, Q74P, and I128A; (h) V69A, Q74P, N88D, and S99P; (i) N30S, V69A, Q74P, and I128T; j) K9T, Q11R, K35R, V69A, and Q74P; (k) AlT, M46L, K49R, E61D, V69A, and H79R; (1) K48E, E68D, N71T, N90H, F103S, and 1114V; (m) S4P, T10A, Q11R, V69A, Q74P, N88D, and T133A; (n) E15K, N30S Y31H, K35R, K48E, V69A, Q74P, and I92T; (o) N30S, E68D, V69A, N71A, Q74P, S75P, K76R, and N90H; (p) N30S, Y31C, T37A, V69A, A73V, Q74P, H79R, and I128T; (q) N26D, N29S, N30S, K54R, E67G, V69A, Q74P, and I92T; (r) K8R, Q13R, N26D, N30T, K35R, T37R, V69A, Q74P, and I92T; and (s) N29S, Y31H, K35R, T37A, K48E, V69A, N71R, Q74P, N88D, and 189V. This approach can also be applied to prepare muteins of other cytokines including interleukins (e.g., IL-2, IL-7, IL-12, IL-15, IL-18, IL-23), interferons (IFNs, including IFNalpha, IFNbeta and IFNgamma), tumor necrosis factors (e.g., TNFalpha, lymphotoxin), transforming growth factors (e.g., TGFbeta1, TGFbeta2, TGFbeta3) and granulocyte macrophage-colony stimulating factor (GM-CS). For example, muteins can be prepared that have desired binding affinity for a cognate receptor.

As noted above, any of the mutant IL-2 polypeptides disclosed herein can include the sequences described; they can also be limited to the sequences described and otherwise identical to SEQ ID NO: 1. Moreover, any of the mutant IL-2 polypeptides disclosed herein can optionally include a substitution of the cysteine residue at position 125 with another residue (e.g., serine) and/or can optionally include a deletion of the alanine residue at position 1 of SEQ ID NO:1.

Another approach to improving the therapeutic index of an IL-2 based therapy is to optimize the pharmacokinetics of the molecule to maximally activate Treg cells. Early studies of IL-2 action demonstrated that IL-2 stimulation of human T cell proliferation in vitro required a minimum of 5-6 hours exposure to effective concentrations of IL-2 (Cantrell, D. A., et. al., 1984, Science, 224: 1312-1316). When administered to human patients, IL-2 has a very short plasma half-life of 85 minutes for intravenous administration and 3.3 hours subcutaneous administration (Kirchner, G. I., et al., 1998, Br J Clin Pharmacol. 46:5-10). Because of its short half-life, maintaining circulating IL-2 at or above the level necessary to stimulate T cell proliferation for the necessary duration necessitates high doses that result in peak IL-2 levels significantly above the EC50 for Treg cells or will require frequent administration. These high IL-2 peak levels can activate IL2Rβγ receptors and have other unintended or adverse effects, for example VLS as noted above. An IL-2 analog, or a multifunctional protein with IL-2 attached to a domain that enables binding to the FcRn receptor, with a longer circulating half-life than IL-2 can achieve a target drug concentration for a specified period of time at a lower dose than IL-2, and with lower peak levels. Such an IL-2 analog will therefore require either lower doses or less frequent administration than IL-2 to effectively stimulate Treg cells. Less frequent subcutaneous administration of an IL-2 drug will also be more tolerable for patients. A therapeutic with these characteristics will translate clinically into improved pharmacological efficacy, reduced toxicity, and improved patient compliance with therapy. Alternatively, IL-2 or muteins of IL-2 (herein, “IL-2*”) can be selectively targeted to the intended site of action (e.g. sites of inflammation or a tumor). This targeting can be achieved by one of several strategies, including the addition of domains to the administered agent that comprise blockers of the IL-2 (or muteins) that are cleaved away or by targeting domains or a combination of the two.

In some embodiments, IL-2* partial agonists can be tailored to bind with higher or lower affinity depending on the desired target; for example, an IL-2* can be engineered to bind with enhanced affinity to one of the receptor subunits and not the others. These types of partial agonists, unlike full agonists or complete antagonists, offer the ability to tune the signaling properties to an amplitude that elicits desired functional properties while not meeting thresholds for undesired properties. Given the differential activities of the partial agonists, a repertoire of IL-2 variants could be engineered to exhibit an even finer degree of distinctive signaling activities, ranging from almost full to partial agonism to complete antagonism.

In some embodiments, the IL-2* has altered affinity for IL-2Rα. In some embodiments, the IL-2* has a higher affinity for IL-2Rα than wild-type IL-2. In other embodiments, the IL-2* has altered affinity for IL-2Rβ. In one embodiment, IL-2* has enhanced binding affinity for IL-2Rβ, e.g., the N-terminus of IL-2Rβ, that eliminates the functional requirement for IL-2Rα. In another embodiment, an IL-2* is generated that has increased binding affinity for IL-2Rβ but that exhibited decreased binding to IL-2Ry, and thereby is defective IL-2Rβry heterodimerization and signaling.

Blocking moieties, described in further detail below, can also be used to favor binding to or activation of one or more receptors. In one embodiment, blocking moieties are added such that IL-2Rβγ binding or activation is blocked but IL-2Rα binding or activation is not changed. In another embodiment, blocking moieties are added such that IL-2Rα binding or activation is diminished. In another embodiment, blocking moieties are added such that binding to and or activation of all three receptors is inhibited. This blocking may be relievable by removal of the blocking moieties in a particular environment, for example by proteolytic cleavage of a linker linking one or more blocking moieties to the cytokine.

A similar approach can be applied to improve other cytokines, particularly for use as immunostimulatory agents, for example for treating cancer. For example, in this aspect, the pharmacokinetics and/or pharmacodynamics of the cytokine (e.g., IL-2, IL-7, IL-12, IL-15, IL-18, IL-21 IL-23, IFNalpha, IFNbeta and IFNgamma, TNFalpha, lymphotoxin, TGFbeta1, TGFbeta2, TGFbeta3 GM-CSF, CXCL10, CCL19, CCL20, and CCL21 can be tailored to maximally activate effector cells (e.g., effect T cells, NK cells) and/or cytotoxic immune response promoting cells (e.g., induce dendritic cell maturation) at a site of desired activity, such as in a tumor, but preferably not systemically.

Thus, provided herein are pharmaceutical compositions comprising at least one cytokine polypeptide, such as interleukins (e.g., IL-2, IL-7, IL-12, IL-15, IL-18, IL-21, IL-23), interferons (IFNs, including IFNalpha, IFNbeta and IFNgamma), tumor necrosis factors (e.g., TNFalpha, lymphotoxin), transforming growth factors (e.g., TGFbeta1, TGFbeta2, TGFbeta3), chemokines (e.g. CXCL10, CCL19, CCL20, CCL21) and granulocyte macrophage-colony stimulating factor (GM-CS) or a functional fragment or mutein of any of the foregoing. The polypeptide typically also includes at least one linker amino acid sequence, wherein the amino acid sequence is in certain embodiments capable of being cleaved by an endogenous protease. In one embodiment, the linker comprises an amino acid sequence comprising HSSKLQ (SEQ ID NO.: 25), GPLGVRG (SEQ ID NO.: 445), IPVSLRSG (SEQ ID NO.: 446), VPLSLYSG (SEQ ID NO. 447), or SGESPAYYTA (SEQ ID NO. 448). In other embodiments, the chimeric polypeptide further contains a blocking moiety, e.g. a steric blocking polypeptide moiety, capable of blocking the activity of the interleukin polypeptide. The blocking moiety, for example, can comprise a human serum albumin (HSA) binding domain or an optionally branched or multi-armed polyethylene glycol (PEG). Alternatively, the pharmaceutical composition comprises a first cytokine polypeptide or a fragment thereof, and blocking moiety, e.g. a steric blocking polypeptide moiety, wherein the blocking moiety blocks the activity of the cytokine polypeptide on the cytokine receptor, and wherein the blocking moiety in certain embodiments comprises a protease cleavable domain. In some embodiments, blockade and reduction of cytokine activity is achieved simply by attaching additional domains with very short linkers to the N or C terminus of the interleukin domain. In such embodiments, it is anticipated the blockade is relieved by protease digestion of the blocking moiety or of the short linker that tethers the blocker to the interleukin. Once the domain is clipped or is released, it will no longer be able to achieve blockade of cytokine activity.

The pharmaceutical composition e.g., chimeric polypeptide can comprise two or more cytokines, which can be the same cytokine polypeptide or different cytokine polypeptides. For example, the two or more different types of cytokines have complementary functions. In some examples, a first cytokine is IL-2 and a second cytokine is IL-12. In some embodiments, each of the two or more different types of cytokine polypeptides have activities that modulate the activity of the other cytokine polypeptides. In some examples of chimeric polypeptides that contain two cytokine polypeptides, a first cytokine polypeptide is T-cell activating, and a second cytokine polypeptide is non-T-cell-activating. In some examples of chimeric polypeptides that contain two cytokine polypeptides, a first cytokine is a chemoattractant, e.g. CXCL10, and a second cytokine is an immune cell activator.

Preferably, the cytokine polypeptides (including functional fragments) that are included in the fusion proteins disclosed herein are not mutated or engineered to alter the properties of the naturally occurring cytokine, including receptor binding affinity and specificity or serum half-life. However, changes in amino acid sequence from naturally occurring (including wild type) cytokine are acceptable to facilitate cloning and to achieve desired expression levels, for example.

### Blocking Moiety

The blocking moiety can be any moiety that inhibits the ability of the cytokine to bind and/or activate its receptor. The blocking moiety can inhibit the ability of the cytokine to bind and/or activate its receptor sterically blocking and/or by noncovalently binding to the cytokine. Examples of suitable blocking moieties include the full length or a cytokine-binding fragment or mutein of the cognate receptor of the cytokine. Antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like that bind the cytokine can also be used. Other suitable antigen-binding domain that bind the cytokine can also be used, include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of suitable blocking polypeptides include polypeptides that sterically inhibit or block binding of the cytokine to its cognate receptor. Advantageously, such moieties can also function as half-life extending elements. For example, a peptide that is modified by conjugation to a water-soluble polymer, such as PEG, can sterically inhibit or prevent binding of the cytokine to its receptor. Polypeptides, or fragments thereof, that have long serum half-lives can also be used, such as serum albumin (human serum albumin), immunoglobulin Fc, transferrin and the like, as well as fragments and muteins of such polypeptides. Antibodies and antigen-binding domains that bind to, for example, a protein with a long serum half-life such as HSA, immunoglobulin or transferrin, or to a receptor that is recycled to the plasma membrane, such as FcRn or transferrin receptor, can also inhibit the cytokine, particularly when bound to their antigen. Examples of such antigen-binding polypeptides include a single chain variable fragment (scFv), single-domain antibody such as a heavy chain variable domain (VH), a light chain variable domain (VL) and a variable domain of camelid-type nanobody (VHH), a dAb and the like. Other suitable antigen-binding domain that bind the cytokine can also be used, include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds.

In illustrative examples, when IL-2 is the cytokine in the chimeric polypeptide, the blocking moiety can be the full length or fragment or mutein of the alpha chain of IL-2 receptor (IL-2Rα) or beta (IL-2Rβ) or gamma chain of IL-2 receptor (IL-2Rγ), an anti-IL-2 single-domain antibody (dAb) or scFv, a Fab, an anti-CD25 antibody or fragment thereof, and anti-HAS dAb or scFv, and the like. In vivo Half-life Extension Elements

Preferably, the chimeric polypeptides comprise an in vivo half-life extension element. Increasing the in vivo half-life of therapeutic molecules with naturally short half-lives allows for a more acceptable and manageable dosing regimen without sacrificing effectiveness. As used herein, a “half-life extension element” is a part of the chimeric polypeptide that increases the in vivo half-life and improve pK, for example, by altering its size (e.g., to be above the kidney filtration cutoff), shape, hydrodynamic radius, charge, or parameters of absorption, biodistribution, metabolism, and elimination. An exemplary way to improve the pK of a polypeptide is by expression of an element in the polypeptide chain that binds to receptors that are recycled to the plasma membrane of cells rather than degraded in the lysosomes, such as the FcRn receptor on endothelial cells and transferrin receptor. Three types of proteins, e.g., human IgGs, HSA (or fragments), and transferrin, persist for much longer in human serum than would be predicted just by their size, which is a function of their ability to bind to receptors that are recycled rather than degraded in the lysosome. These proteins, or fragments of them that retain the FcRn binding are routinely linked to other polypeptides to extend their serum half-life. In one embodiment, the half-life extension element is a human serum albumin (HSA) binding domain. HSA (SEQ ID NO: 2) may also be directly bound to the pharmaceutical compositions or bound via a short linker. Fragments of HSA may also be used. HSA and fragments thereof can function as both a blocking moiety and a half-life extension element. Human IgGs and Fc fragments can also carry out a similar function.

The serum half-life extension element can also be antigen-binding polypeptide that binds to a protein with a long serum half-life such as serum albumin, transferrin and the like. Examples of such polypeptides include antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like. Other suitable antigen-binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of antigen-binding polypeptides include a ligand for a desired receptor, a ligand-binding portion of a receptor, a lectin, and peptides that binds to or associates with one or more target antigens.

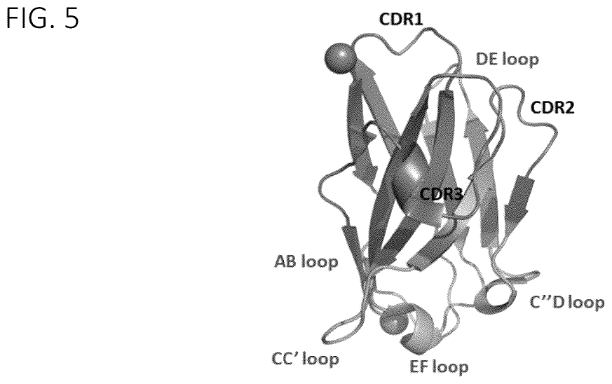

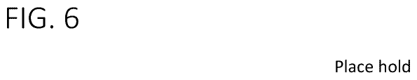

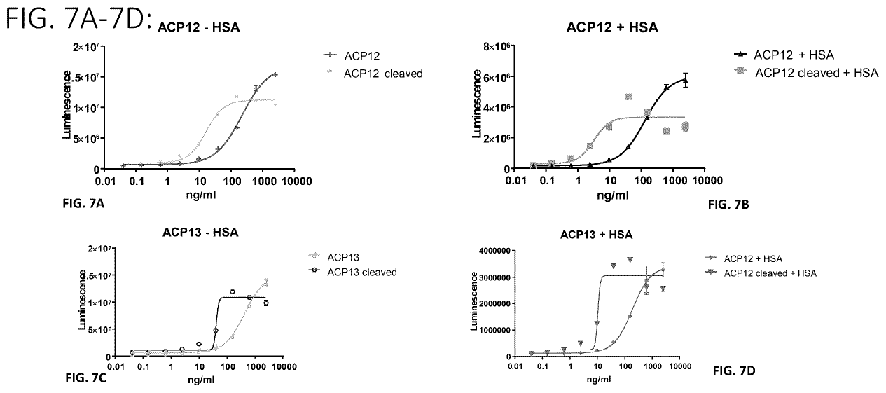

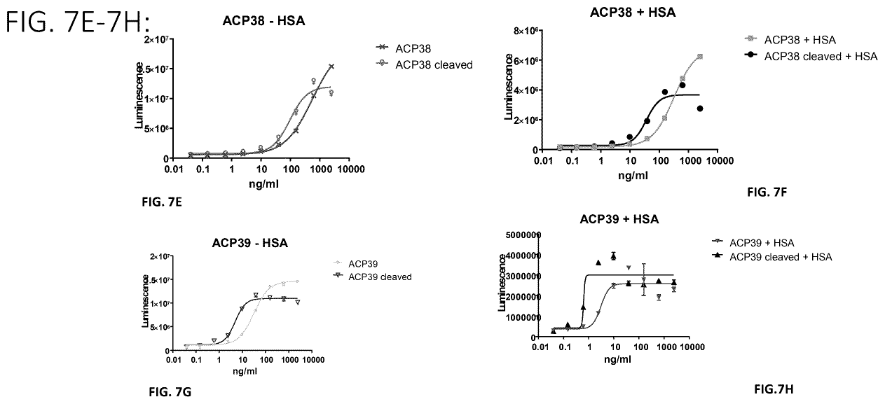

Some preferred serum half-life extension elements are polypeptides that comprise complementarity determining regions (CDRs), and optionally non-CDR loops. Advantageously, such serum half-life extension elements can extend the serum half-life of the cytokine, and also function as inhibitors of the cytokine (e.g., via steric blocking, non-covalent interaction or combination thereof) and/or as targeting domains. In some instances, the serum half-life extension elements are domains derived from an immunoglobulin molecule (Ig molecule) or engineered protein scaffolds that mimic antibody structure and/or binding activity. The Ig may be of any class or subclass (IgG1, IgG2, IgG3, IgG4, IgA, IgE, IgM etc). A polypeptide chain of an Ig molecule folds into a series of parallel beta strands linked by loops. In the variable region, three of the loops constitute the “complementarity determining regions” (CDRs) which determine the antigen binding specificity of the molecule. An IgG molecule comprises at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs) with are hypervariable in sequence and/or involved in antigen recognition and/or usually form structurally defined loops, interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments of this disclosure, at least some or all of the amino acid sequences of FR1, FR2, FR3, and FR4 are part of the “non-CDR loop” of the binding moieties described herein. As shown in FIG. 5, a variable domain of an immunoglobulin molecule has several beta strands that are arranged in two sheets. The variable domains of both light and heavy immunoglobulin chains contain three hypervariable loops, or complementarity-determining regions (CDRs). The three CDRs of a V domain (CDR1, CDR2, CDR3) cluster at one end of the beta barrel. The CDRs are the loops that connect beta strands B-C, C′-C″, and F-G of the immunoglobulin fold, whereas the bottom loops that connect beta strands AB, CC′, C″-D and E-F of the immunoglobulin fold, and the top loop that connects the D-E strands of the immunoglobulin fold are the non-CDR loops. In some embodiments of this disclosure, at least some amino acid residues of a constant domain, CH1, CH2, or CH3, are part of the “non-CDR loop” of the binding moieties described herein. Non-CDR loops comprise, in some embodiments, one or more of AB, CD, EF, and DE loops of a Cl-set domain of an Ig or an Ig-like molecule; AB, CC′, EF, FG, BC, and EC′ loops of a C2-set domain of an Ig or an Ig-like molecule; DE, BD, GF, A(A1A2)B, and EF loops of I(Intermediate)-set domain of an Ig or Ig-like molecule.

Within the variable domain, the CDRs are believed to be responsible for antigen recognition and binding, while the FR residues are considered a scaffold for the CDRs. However, in certain cases, some of the FR residues play an important role in antigen recognition and binding. Framework region residues that affect Ag binding are divided into two categories. The first are FR residues that contact the antigen, thus are part of the binding-site, and some of these residues are close in sequence to the CDRs. Other residues are those that are far from the CDRs in sequence, but are in close proximity to it in the 3-D structure of the molecule, e.g., a loop in heavy chain.

The binding moieties are any kinds of polypeptides. For example, in certain instances the binding moieties are natural peptides, synthetic peptides, or fibronectin scaffolds, or engineered bulk serum proteins. The bulk serum protein comprises, for example, albumin, fibrinogen, or a globulin. In some embodiments, the binding moieties are engineered scaffolds. Engineered scaffolds comprise, for example, sdAb, a scFv, a Fab, a VHH, a fibronectin type III domain, immunoglobulin-like scaffold (as suggested in Halaby et al., 1999. Prot Eng 12(7):563-571), DARPin, cystine knot peptide, lipocalin, three-helix bundle scaffold, protein G-related albumin-binding module, or a DNA or RNA aptamer scaffold.

In some cases, the serum half-life extending element comprises a binding site for a bulk serum protein. In some embodiments, the CDRs provide the binding site for the bulk serum protein. The bulk serum protein is, in some examples, a globulin, albumin, transferrin, IgG1, IgG2, IgG4, IgG3, IgA monomer, Factor XIII, Fibrinogen, IgE, or pentameric IgM. In some embodiments, the CDR form a binding site for an immunoglobulin light chain, such as an Igκ free light chain or an Ig, free light chain.

The serum half-life extension element can be any type of binding domain, including but not limited to, domains from a monoclonal antibody, a polyclonal antibody, a recombinant antibody, a human antibody, a humanized antibody. In some embodiments, the binding moiety is a single chain variable fragment (scFv), single-domain antibody such as a heavy chain variable domain (VH), a light chain variable domain (VL) and a variable domain (VHH) of camelid derived nanobody. In other embodiments, the binding moieties are non-Ig binding domains, i.e., antibody mimetic, such as anticalins, affilins, affibody molecules, affimers, affitins, alphabodies, avimers, DARPins, fynomers, kunitz domain peptides, and monobodies.

In other embodiments, the serum half-life extension element can be a water-soluble polymer or a peptide that is conjugated to a water-soluble polymer, such as PEG. “PEG,” “polyethylene glycol” and “poly(ethylene glycol)” as used herein, are interchangeable and encompass any nonpeptidic water-soluble poly(ethylene oxide). The term “PEG” also means a polymer that contains a majority, that is to say, greater than 50%, of —OCH₂CH₂— repeating subunits. With respect to specific forms, the PEG can take any number of a variety of molecular weights, as well as structures or geometries such as “branched,” “linear,” “forked,” “multifunctional,” and the like, to be described in greater detail below. The PEG is not limited to a particular structure and can be linear (e.g., an end capped, e.g., alkoxy PEG or a bifunctional PEG), branched or multi-armed (e.g., forked PEG or PEG attached to a polyol core), a dendritic (or star) architecture, each with or without one or more degradable linkages. Moreover, the internal structure of the PEG can be organized in any number of different repeat patterns and can be selected from the group consisting of homopolymer, alternating copolymer, random copolymer, block copolymer, alternating tripolymer, random tripolymer, and block tripolymer. PEGs can be conjugated to polypeptide and peptides through any suitable method. Typically a reactive PEG derivative, such as N-hydroxysuccinamidyl ester PEG, is reacted with a peptide or polypeptide that includes amino acids with a side chain that contains an amine, sulfhydryl, carboxylic acid or hydroxyl functional group, such as cysteine, lysine, asparagine, glutamine, theonine, tyrosine, serine, aspartic acid, and glutamic acid.

### Targeting and Retention Domains

For certain applications, it may be desirable to maximize the amount of time the construct is present in its desired location in the body. This can be achieved by including one further domain in the chimeric polypeptide (fusion protein) to influence its movements within the body. For example, the chimeric nucleic acids can encode a domain that directs the polypeptide to a location in the body, e.g., tumor cells or a site of inflammation; this domain is termed a “targeting domain” and/or encode a domain that retains the polypeptide in a location in the body, e.g., tumor cells or a site of inflammation; this domain is termed a “retention domain”. In some embodiments a domain can function as both a targeting and a retention domain. In some embodiments, the targeting domain and/or retention domain are specific to a protease-rich environment. In some embodiments, the encoded targeting domain and/or retention domain are specific for regulatory T cells (Tregs), for example targeting the CCR4 or CD39 receptors. Other suitable targeting and/or retention domains comprise those that have a cognate ligand that is overexpressed in inflamed tissues, e.g., the IL-1 receptor, or the IL-6 receptor. In other embodiments, the suitable targeting and/or retention domains comprise those who have a cognate ligand that is overexpressed in tumor tissue, e.g., Epcam, CEA or mesothelin. In some embodiments, the targeting domain is linked to the interleukin via a linker which is cleaved at the site of action (e.g. by inflammation or cancer specific proteases) releasing the interleukin full activity at the desired site. In some embodiments, the targeting and/or retention domain is linked to the interleukin via a linker which is not cleaved at the site of action (e.g. by inflammation or cancer specific proteases), causing the cytokine to remain at the desired site.

Antigens of choice, in some cases, are expressed on the surface of a diseased cell or tissue, for example a tumor or a cancer cell. Antigens useful for tumor targeting and retention include but are not limited to EpCAM, EGFR, HER-2, HER-3, c-Met, FOLR1, and CEA. Pharmaceutical compositions disclosed herein, also include proteins comprising two targeting and/or retention domains that bind to two different target antigens known to be expressed on a diseased cell or tissue. Exemplary pairs of antigen binding domains include but are not limited to EGFR/CEA, EpCAM/CEA, and HER-2/HER-3.

Suitable targeting and/or retention domains include antigen-binding domains, such as antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like. Other suitable antigen-binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of antigen-binding polypeptides include a ligand for a desired receptor, a ligand-binding portion of a receptor, a lectin, and peptides that binds to or associates with one or more target antigens.

In some embodiments, the targeting and/or retention domains specifically bind to a cell surface molecule. In some embodiments, the targeting and/or retention domains specifically bind to a tumor antigen. In some embodiments, the targeting polypeptides specifically and independently bind to a tumor antigen selected from at least one of Fibroblast activation protein alpha (FAPa), Trophoblast glycoprotein (5T4), Tumor-associated calcium signal transducer 2 (Trop2), Fibronectin EDB (EDB-FN), fibronectin EIIIB domain, CGS-2, EpCAM, EGFR, HER-2, HER-3, cMet, CEA, and FOLR1. In some embodiments, the targeting polypeptides specifically and independently bind to two different antigens, wherein at least one of the antigens is a tumor antigen selected from EpCAM, EGFR, HER-2, HER-3, cMet, CEA, and FOLR1.

The targeting and/or retention antigen can be a tumor antigen expressed on a tumor cell. Tumor antigens are well known in the art and include, for example, EpCAM, EGFR, HER-2, HER-3, c-Met, FOLR1, PSMA, CD38, BCMA, and CEA. 5T4, AFP, B7-H3, Cadherin-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16E6, HPV-16E7, ITGA2, ITGA3, SLC39A6, MAGE, mesothelin, Mucd, Muc16, NaPi2b, Nectin-4, P-cadherin, NY-ESO-1, PRLR, PSCA, PTK7, ROR1, SLC44A4, SLTRK5, SLTRK6, STEAPI, TIM1, Trop2, WT1.

The targeting and/or retention antigen can be an immune checkpoint protein. Examples of immune checkpoint proteins include but are not limited to CD27, CD137, 2B4, TIGIT, CD155, ICOS, HVEM, CD40L, LIGHT, TIM-1, OX40, DNAM-1, PD-L1, PD1, PD-L2, CTLA-4, CD8, CD40, CEACAMI, CD48, CD70, A2AR, CD39, CD73, B7-H3, B7-H4, BTLA, IDO1, IDO2, TDO, KIR, LAG-3, TIM-3, or VISTA.

The targeting and/or retention antigen can be a cell surface molecule such as a protein, lipid or polysaccharide. In some embodiments, a targeting and/or retention antigen is a on a tumor cell, virally infected cell, bacterially infected cell, damaged red blood cell, arterial plaque cell, inflamed or fibrotic tissue cell. The targeting and/or retention antigen can comprise an immune response modulator. Examples of immune response modulator include but are not limited to granulocyte-macrophage colony stimulating factor (GM-CSF), macrophage colony stimulating factor (M-CSF), granulocyte colony stimulating factor (G-CSF), interleukin 2 (IL-2), interleukin 3 (IL-3), interleukin 12 (IL-12), interleukin 15 (IL-15), B7-1 (CD80), B7-2 (CD86), GITRL, CD3, or GITR.

The targeting and/or retention antigen can be a cytokine receptor. Examples, of cytokine receptors include but are not limited to Type I cytokine receptors, such as GM-CSF receptor, G-CSF receptor, Type I IL receptors, Epo receptor, LIF receptor, CNTF receptor, TPO receptor; Type II Cytokine receptors, such as IFN-alpha receptor (IFNAR1, IFNAR2), IFB-beta receptor, IFN-gamma receptor (IFNGRI, IFNGR2), Type II IL receptors; chemokine receptors, such as CC chemokine receptors, CXC chemokine receptors, CX3C chemokine receptors, XC chemokine receptors; tumor necrosis receptor superfamily receptors, such as TNFRSF5/CD40, TNFRSF8/CD30, TNFRSF7/CD27, TNFRSF1A/TNFR1/CD120a, TNFRSF1B/TNFR2/CD120b; TGF-beta receptors, such as TGF-beta receptor 1, TGF-beta receptor 2; Ig super family receptors, such as IL-1 receptors, CSF-1R, PDGFR (PDGFRA, PDGFRB), SCFR.

### Linkers

As stated above, the pharmaceutical compositions comprise one or more linker sequences. A linker sequence serves to provide flexibility between polypeptides, such that, for example, the blocking moiety is capable of inhibiting the activity of the cytokine polypeptide. The linker sequence can be located between any or all of the cytokine polypeptide, the serum half-life extension element, and/or the blocking moiety. As described herein at least one of the linkers is protease cleavable, and contains a (one or more) cleavage site for a (one or more) desired protease. Preferably, the desired protease is enriched or selectively expressed at the desired site of cytokine activity (e.g., the tumor microenvironment). Thus, the fusion protein is preferentially or selectively cleaved at the site of desired cytokine activity.

Suitable linkers can be of different lengths, such as from 1 amino acid (e.g., Gly) to 20 amino acids, from 2 amino acids to 15 amino acids, from 3 amino acids to 12 amino acids, including 4 amino acids to 10 amino acids, amino acids to 9 amino acids, 6 amino acids to 8 amino acids, or 7 amino acids to 8 amino acids, and may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids.

The orientation of the components of the pharmaceutical composition, are largely a matter of design choice and it is recognized that multiple orientations are possible and all are intended to be encompassed by this disclosure. For example, a blocking moiety can be located C-terminally or N-terminally to a cytokine polypeptide.

Proteases known to be associated with diseased cells or tissues include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mirl-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, urokinase plasminogen activator (uPA), enterokinase, prostate-specific antigen (PSA, hK3), interleukin-I1β converting enzyme, thrombin, FAP (FAP-a), dipeptidyl peptidase, meprins, granzymes and dipeptidyl peptidase IV (DPPJV/CD26). Proteases capable of cleaving amino acid sequences encoded by the chimeric nucleic acid sequences provided herein can, for example, be selected from the group consisting of a prostate specific antigen (PSA), a matrix metalloproteinase (MMP), an A Disintigrin and a Metalloproteinase (ADAM), a plasminogen activator, a cathepsin, a caspase, a tumor cell surface protease, and an elastase. The MMP can, for example, be matrix metalloproteinase 2 (MMP2) or matrix metalloproteinase 9 (MMP9).

Proteases useful in the methods disclosed herein are presented in Table 1, and exemplary proteases and their cleavage site are presented in Table 1a:

TABLE 1
Proteases relevant to inflammation and cancer

| | | |
|---|---|---|
| Protease | Specificity | Other aspects |

Secreted by killer T cells:

| | | |
|---|---|---|
| Granzyme B (grB) | Cleaves after Asp | Type of serine protease; strongly |
| | residues (asp-ase) | implicated in inducing perforin-dependent |
| | | target cell apoptosis |
| Granzyme A (grA) | trypsin-like, cleaves after | Type of serine protease; |
| | basic residues | |
| Granzyme H (grH) | Unknown substrate | Type of serine protease; |
| | specificity | Other granzymes are also secreted by |
| | | killer T cells, but not all are present in |
| | | humans |
| Caspase-8 | Cleaves after Asp | Type of cysteine protease; plays essential |
| | residues | role in TCR-induced cellular expansion- |
| | | exact molecular role unclear |
| Mucosa-associated | Cleaves after arginine | Type of cysteine protease; likely acts both |
| lymphoid tissue | residues | as a scaffold and proteolytically active |
| (MALT1) | | enzyme in the CBM-dependent signaling |
| | | pathway |
| Tryptase | Targets: angiotensin I, | Type of mast cell-specific serine protease; |
| | fibrinogen, prourokinase, | trypsin-like; resistant to inhibition by |
| | TGFβ; preferentially | macromolecular protease inhibitors |
| | cleaves proteins after | expressed in mammals due to their |
| | lysine or arginine | tetrameric structure, with all sites facing |
| | residues | narrow central pore; also associated with |
| | | inflammation |

Associated with inflammation:

| | | |
|---|---|---|
| Thrombin | Targets: FGF-2, | Type of serine protease; modulates |
| | HB-EGF, Osteo-pontin, | activity of vascular growth factors, |
| | PDGF, VEGF | chemokines and extracellular proteins; |
| | | strengthens VEGF-induced proliferation; |
| | | induces cell migration; angiogenic factor; |
| | | regulates hemostasis |
| Chymase | Exhibit chymotrypsin- | Type of mast cell-specific serine protease |
| | like specificity, cleaving | |
| | proteins after aromatic | |
| | amino acid residues | |
| Carboxypeptidase A | Cleaves amino acid | Type of zinc-dependent metalloproteinase |
| (MC-CPA) | residues from C-terminal | |
| | end of peptides and | |
| | proteins | |
| Kallikreins | Targets: high molecular | Type of serine protease; modulate |
| | weight | relaxation response; contribute to |
| | kininogen, pro-urokinase | inflammatory response; fibrin degradation |
| Elastase | Targets: E-cadherin, GM- | Type of neutrophil serine protease; |
| | CSF, IL-1, IL-2, IL-6, | degrades ECM components; regulates |
| | IL8, p38^{MAPK}, TNFα, VE- | inflammatory response; activates pro- |
| | cadherin | apoptotic signaling |
| Cathepsin G | Targets: EGF, ENA-78, | Type of serine protease; degrades ECM |
| | IL-8, MCP-1, MMP-2, | components; chemo-attractant of |
| | MT1-MMP, | leukocytes; regulates inflammatory |
| | PAI-1, RANTES, TGFβ, | response; promotes apoptosis |
| | TNFα | |
| PR-3 | Targets: ENA-78, IL-8, | Type of serine protease; promotes |
| | IL-18, JNK, p38^{MAPK}, | inflammatory response; activates pro- |
| | TNFα | apoptotic signaling |
| Granzyme M (grM) | Cleaves after Met and | Type of serine protease; only expressed in |
| | other long, unbranched | NK cells |
| | hydrophobic residues | |
| Calpains | Cleave between Arg and | Family of cysteine proteases; calcium- |
| | Gly | dependent; activation is involved in the |
| | | process of numerous inflammation- |
| | | associated diseases |

TABLE 1a
Exemplary Proteases and Protease Recognition
Sequences

| | Cleavage | SEQ |
|---|---|---|
| | Domain | ID |
| Protease | Sequence | NO: |
| MMP7 | KRALGLPG | 3 |
| MMP7 | (DE)₈RPLALWRS(DR)₈ | 4 |
| MMP9 | PR(S/T)(L/I)(S/T) | 5 |
| MMP9 | LEATA | 6 |
| MMP11 | GGAANLVRGG | 7 |
| MMP14 | SGRIGFLRTA | 8 |
| MMP | PLGLAG | 9 |
| MMP | PLGLAX | 10 |
| MMP | PLGC(me)AG | 11 |
| MMP | ESPAYYTA | 12 |
| MMP | RLQLKL | 13 |
| MMP | RLQLKAC | 14 |
| MMP2, MMP9, MMP14 | EP(Cit)G(Hof)YL | 15 |
| Urokinase plasminogen | SGRSA | 16 |
| activator (uPA) | | |
| Urokinase plasminogen | DAFK | 17 |
| activator (uPA) | | |
| Urokinase plasminogen | GGGRR | 18 |
| activator (uPA) | | |
| Lysosomal Enzyme | GFLG | 19 |
| Lysosomal Enzyme | ALAL | 20 |
| Lysosomal Enzyme | FK | 21 |
| Cathepsin B | NLL | 22 |
| Cathepsin D | PIC(Et)FF | 23 |
| Cathepsin K | GGPRGLPG | 24 |
| Prostate Specific | HSSKLQ | 25 |
| Antigen | | |
| Prostate Specific | HSSKLQL | 26 |
| Antigen | | |
| Prostate Specific | HSSKLQEDA | 27 |
| Antigen | | |
| Herpes Simplex Virus | LVLASSSFGY | 28 |
| Protease | | |
| HIV Protease | GVSQNYPIVG | 29 |
| CMV Protease | GVVQASCRLA | 30 |
| Thrombin | F(Pip)RS | 31 |
| Thrombin | DPRSFL | 32 |
| Thrombin | PPRSFL | 33 |
| Caspase-3 | DEVD | 34 |
| Caspase-3 | DEVDP | 35 |
| Caspase-3 | KGSGDVEG | 36 |
| Interleukin 1β | GWEHDG | 37 |
| converting enzyme | | |
| Enterokinase | EDDDDKA | 38 |
| FAP | KQEQNPGST | 39 |
| Kallikrein 2 | GKAFRR | 40 |
| Plasmin | DAFK | 41 |
| Plasmin | DVLK | 42 |
| Plasmin | DAFK | 43 |
| TOP | ALLLALL | 44 |

Provided herein are pharmaceutical compositions comprising polypeptide sequences. As with all peptides, polypeptides, and proteins, including fragments thereof, it is understood that additional modifications in the amino acid sequence of the chimeric polypeptides (amino acid sequence variants) can occur that do not alter the nature or function of the peptides, polypeptides, or proteins. Such modifications include conservative amino acid substitutions and are discussed in greater detail below. 11591 The compositions provided herein have a desired function. The compositions are comprised of at least a cytokine polypeptide, such as JL-2, JL-7, JL-12, JL-15, JL-18, JL-21, IFNa, or IFNγ, or a chemokine, such as CXCL10, CCL19, CCL20, CCL21, a blocking moiety, e.g. a steric blocking polypeptide, and an optional serum half-life extension element, and an optional targeting polypeptide, with one or more linkers connecting each polypeptide in the composition. The first polypeptide, e.g., an IL-2 mutein, is provided to be an active agent. The blocking moiety is provided to block the activity of the interleukin. The linker polypeptide, e.g., a protease cleavable polypeptide, is provided to be cleaved by a protease that is specifically expressed at the intended target of the active agent. Optionally, the blocking moiety blocks the activity of the first polypeptide by binding the interleukin polypeptide. In some embodiments, the blocking moiety, e.g. a steric blocking peptide, is linked to the interleukin via a protease-cleavable linker which is cleaved at the site of action (e.g. by inflammation-specific or tumor-specific proteases) releasing the cytokine full activity at the desired site.

The protease cleavage site may be a naturally occurring protease cleavage site or an artificially engineered protease cleavage site. The artificially engineered protease cleavage site can be cleaved by more than one protease specific to the desired environment in which cleavage will occur, e.g. a tumor. The protease cleavage site may be cleavable by at least one protease, at least two proteases, at least three proteases, or at least four proteases.

In some embodiments, the linker comprises glycine-glycine, a sortase-recognition motif, or a sortase-recognition motif and a peptide sequence (Gly₄Ser)ₙ (SEQ ID NO.: 443) or (Gly₃Ser)ₙ, (SEQ ID NO.: 444), wherein n is 1, 2, 3, 4 or 5. In one embodiment, the sortase-recognition motif comprises a peptide sequence LPXTG (SEQ ID NO.: 442), where X is any amino acid. In one embodiment, the covalent linkage is between a reactive lysine residue attached to the C-terminal of the cytokine polypeptide and a reactive aspartic acid attached to the N-terminal of the blocking or other moiety. In one embodiment, the covalent linkage is between a reactive aspartic acid residue attached to the N-terminal of the cytokine polypeptide and a reactive lysine residue attached to the C-terminal of the blocking or other moiety.

### Cleavage and Inducibility

As described herein, the activity of the cytokine polypeptide the context of the fusion protein is attenuated, and protease cleavage at the desired site of activity, such as in a tumor microenvironment, releases a form of the cytokine from the fusion protein that is much more active as a cytokine receptor agonist than the fusion protein. For example, the cytokine-receptor activating (agonist) activity of the fusion polypeptide can be at least about 10×, at least about 50×, at least about 100×, at least about 250×, at least about 500×, or at least about 1000× less than the cytokine receptor activating activity of the cytokine polypeptide as a separate molecular entity. The cytokine polypeptide that is part of the fusion protein exists as a separate molecular entity when it contains an amino acid that is substantially identical to the cytokine polypeptide and does not substantially include additional amino acids and is not associated (by covalent or non-covalent bonds) with other molecules. If necessary, a cytokine polypeptide as a separate molecular entity may include some additional amino acid sequences, such as a tag or short sequence to aid in expression and/or purification.

In other examples, the cytokine-receptor activating (agonist) activity of the fusion polypeptide is at least about 10×, at least about 50×, at least about 100×, at least about 250×, at least about 500×, or about 1000× less than the cytokine receptor activating activity of the polypeptide that contains the cytokine polypeptide that is produced by cleavage of the protease cleavable linker in the fusion protein. In other words, the cytokine receptor activating (agonist) activity of the polypeptide that contains the cytokine polypeptide that is produced by cleavage of the protease cleavable linker in the fusion protein is at least about 10×, at least about 50×, at least about 100×, at least about 250×, at least about 500×, or at least about 1000× greater than the cytokine receptor activating activity of the fusion protein.

### Polypeptide Substitutions

The polypeptides described herein can include components (e.g., the cytokine, the blocking moiety) that have the same amino acid sequence of the corresponding naturally occurring protein (e.g., IL-2, IL-15, HSA) or can have an amino acid sequence that differs from the naturally occurring protein so long as the desired function is maintained. It is understood that one way to define any known modifications and derivatives or those that might arise, of the disclosed proteins and nucleic acids that encode them is through defining the sequence variants in terms of identity to specific known reference sequences. Specifically disclosed are polypeptides and nucleic acids which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to the chimeric polypeptides provided herein. For example, provided are polypeptides or nucleic acids that have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to the sequence of any of the nucleic acids or polypeptides described herein. Those of skill in the art readily understand how to determine the identity of two polypeptides or two nucleic acids. For example, the identity can be calculated after aligning the two sequences so that the identity is at its highest level.

Another way of calculating identity can be performed by published algorithms. Optimal alignment of sequences for comparison may be conducted by the local identity algorithm of Smith and Waterman Adv. Appl. Math. 2:482 (1981), by the identity alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of identity can be obtained for nucleic acids by, for example, the algorithms disclosed in Zuker, Science 244:48-52 (1989); Jaeger et al., Proc. Natl. Acad. Sci. USA 86:7706-7710 (1989); Jaeger et al., Methods Enzymol. 183:281-306 (1989), which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

Protein modifications include amino acid sequence modifications. Modifications in amino acid sequence may arise naturally as allelic variations (e.g., due to genetic polymorphism), may arise due to environmental influence (e.g., by exposure to ultraviolet light), or may be produced by human intervention (e.g., by mutagenesis of cloned DNA sequences), such as induced point, deletion, insertion and substitution mutants. These modifications can result in changes in the amino acid sequence, provide silent mutations, modify a restriction site, or provide other specific mutations. Amino acid sequence modifications typically fall into one or more of three classes: substitutional, insertional or deletional modifications. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional modifications are those in which at least one residue has been removed and a different residue inserted in its place. Such substitutions generally are made in accordance with the following Table 2 and are referred to as conservative substitutions.

TABLE 2
Exemplary amino acid substitutions

| Amino Acid | Exemplary Substitutions |
|---|---|
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

Modifications, including the specific amino acid substitutions, are made by known methods. For example, modifications are made by site specific mutagenesis of nucleotides in the DNA encoding the polypeptide, thereby producing DNA encoding the modification, and thereafter expressing the DNA in recombinant cell culture. Techniques for making substitution mutations at predetermined sites in DNA having a known sequence are well known, for example M13 primer mutagenesis and PCR mutagenesis.

Modifications can be selected to optimize binding. For example, affinity maturation techniques can be used to alter binding of the scFv by introducing random mutations inside the complementarity determining regions (CDRs). Such random mutations can be introduced using a variety of techniques, including radiation, chemical mutagens or error-prone PCR. Multiple rounds of mutation and selection can be performed using, for example, phage display.

The disclosure also relates to nucleic acids that encode the chimeric polypeptides described herein, and to the use of such nucleic acids to produce the chimeric polypeptides and for therapeutic purposes. For example, the invention includes DNA and RNA molecules (e.g., mRNA, self-replicating RNA) that encode a chimeric polypeptide and to the therapeutic use of such DNA and RNA molecules.

### Exemplary Compositions

Exemplary fusion proteins of the invention combine the above described elements in a variety of orientations. The orientations described in this section are meant as examples and are not to be considered limiting.

In some embodiments, the fusion protein comprises a cytokine, a blocking moiety and a half-life extension element. In some embodiments, the cytokine is positioned between the half-life extension element and the blocking moiety. In some embodiments, the cytokine is N-terminal to the blocking moiety and the half-life extension element. In some such embodiments, the cytokine is proximal to the blocking moiety; in some such embodiments, the cytokine is proximal to the half-life extension element. At least one protease-cleavable linker must be included in all embodiments, such that the cytokine may be active upon cleavage. In some embodiments, the cytokine is C-terminal to the blocking moiety and the half-life extension element. Additional elements may be attached to one another by a cleavable linker, a non-cleavable linker, or by direct fusion.

In some embodiments, the blocking domains used are capable of extending half-life, and the cytokine is positioned between two such blocking domains. In some embodiments, the cytokine is positioned between two blocking domains, one of which is capable of extending half-life.

In some embodiments, two cytokines are included in the same construct. In some embodiments, the cytokines are connected to two blocking domains each (three in total in one molecule), with a blocking domain between the two cytokine domains. In some embodiments, one or more additional half-life extension domains may be included to optimize pharmacokinetic properties. In some cases, it is beneficial to include two of the same cytokine to facilitate dimerization. An example of a cytokine that works as a dimer is IFN H.

In some embodiments, three cytokines are included in the same construct. In some embodiments, the third cytokine may function to block the other two in place of a blocking domain between the two cytokines.

Preferred half-life extension elements for use in the fusion proteins are human serum albumin (HSA), an antibody or antibody fragment (e.g., scFV, dAb) which binds serum albumin, a human or humanized IgG, or a fragment of any of the foregoing. In some preferred embodiments, the blocking moiety is human serum albumin (HSA), or an antibody or antibody fragment which binds serum albumin, an antibody which binds the cytokine and prevents activation of binding or activation of the cytokine receptor, another cytokine, or a fragment of any of the foregoing. In preferred embodiments comprising an additional targeting domain, the targeting domain is an antibody which binds a cell surface protein which is enriched on the surface of cancer cells, such as EpCAM, FOLR1, and Fibronectin.

Methods of treatment and Pharmaceutical Compositions

Further provided are methods of treating a subject with or at risk of developing an of a disease or disorder, such as proliferative disease, a tumorous disease, an inflammatory disease, an immunological disorder, an autoimmune disease, an infectious disease, a viral disease, an allergic reaction, a parasitic reaction, or graft-versus-host disease. The methods administering to a subject in need thereof an effective amount of a fusion protein as disclosed herein that is typically administered as a pharmaceutical composition. In some embodiments, the method further comprises selecting a subject with or at risk of developing such a disease or disorder. The pharmaceutical composition preferably comprises a blocked cytokine, fragment or mutein thereof that is activated at a site of inflammation or a tumor. In one embodiment, the chimeric polypeptide comprises a cytokine polypeptide, fragment or mutein thereof and a serum half-life extension element. In another embodiment, the chimeric polypeptide comprises a cytokine polypeptide, fragment or mutein thereof and a blocking moiety, e.g. a steric blocking polypeptide, wherein the steric blocking polypeptide is capable of sterically blocking the activity of the cytokine polypeptide, fragment or mutein thereof. In another embodiment, the chimeric polypeptide comprises a cytokine polypeptide, fragment or mutein thereof, a blocking moiety, and a serum half-life extension element.

Inflammation is part of the complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and to initiate tissue repair. Inflammation can occur from infection, as a symptom or a disease, e.g., cancer, atherosclerosis, allergies, myopathies, HIV, obesity, or an autoimmune disease. An autoimmune disease is a chronic condition arising from an abnormal immune response to a self-antigen. Autoimmune diseases that may be treated with the polypeptides disclosed herein include but are not limited to lupus, celiac disease, diabetes mellitus type 1, Graves disease, inflammatory bowel disease, multiple sclerosis, psoriasis, rheumatoid arthritis, and systemic lupus erythematosus.

The pharmaceutical composition can comprise one or more protease-cleavable linker sequences. The linker sequence serves to provide flexibility between polypeptides, such that each polypeptide is capable of inhibiting the activity of the first polypeptide. The linker sequence can be located between any or all of the cytokine polypeptide, fragment or mutein thereof, the blocking moiety, and serum half-life extension element. Optionally, the composition comprises, two, three, four, or five linker sequences. The linker sequence, two, three, or four linker sequences can be the same or different linker sequences. In one embodiment, the linker sequence comprises GGGGS (SEQ ID NO.: 449), GSGSGS (SEQ ID NO.: 450), or G(SGGG)₂SGGT (SEQ ID NO.: 451). In another embodiment, the linker comprises a protease-cleavable sequence selected from group consisting of HSSKLQ (SEQ ID NO.: 25), GPLGVRG (SEQ ID NO.: 445), IPVSLRSG (SEQ ID NO.: 446), VPLSLYSG (SEQ ID NO.: 447, and SGESPAYYTA (SEQ ID NO.: 448).

In some embodiments, the linker is cleaved by a protease selected from the group consisting of a kallikrein, thrombin, chymase, carboxypeptidase A, cathepsin G, an elastase, PR-3, granzyme M, a calpain, a matrix metalloproteinase (MMP), a plasminogen activator, a cathepsin, a caspase, a tryptase, or a tumor cell surface protease.

Suitable linkers can be of different lengths, such as from 1 amino acid (e.g., Gly) to 20 amino acids, from 2 amino acids to 15 amino acids, from 3 amino acids to 12 amino acids, including 4 amino acids to 10 amino acids, amino acids to 9 amino acids, 6 amino acids to 8 amino acids, or 7 amino acids to 8 amino acids, and may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids.

Further provided are methods of treating a subject with or at risk of developing cancer. The methods comprise administering to the subject in need thereof an effective amount of a chimeric polypeptide (a fusion protein) as disclosed herein that is typically administered as a pharmaceutical composition. In some embodiments, the method further comprises selecting a subject with or at risk of developing cancer. The pharmaceutical composition preferably comprises a blocked cytokine, fragment or mutein thereof that is activated at a tumor site. Preferably, the tumor is a solid tumor. The cancer may be, but not limited to, a colon cancer, a lung cancer, a melanoma, a sarcoma, a renal cell carcinoma, and a breast cancer.

The method can further involve the administration of one or more additional agents to treat cancer, such as chemotherapeutic agents (e.g., Adriamycin, Cerubidine, Bleomycin, Alkeran, Velban, Oncovin, Fluorouracil, Thiotepa, Methotrexate, Bisantrene, Noantrone, Thiguanine, Cytaribine, Procarabizine), immuno-oncology agents (e.g., anti-PD-L1, anti-CTLA4, anti-PD-1, anti-CD47, anti-GD2), cellular therapies (e.g, CAR-T, T-cell therapy), oncolytic viruses and the like.

Provided herein are pharmaceutical formulations or compositions containing the chimeric polypeptides and a pharmaceutically acceptable carrier. The herein provided compositions are suitable for administration in vitro or in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical formulation or composition in which it is contained. The carrier is selected to minimize degradation of the active ingredient and to minimize adverse side effects in the subject.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic, although the formulate can be hypertonic or hypotonic if desired. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the chimeric polypeptides or nucleic acid sequences encoding the chimeric polypeptides to humans or other subjects.

The pharmaceutical formulations or compositions are administered in a number of ways depending on whether local or systemic treatment is desired and on the area to be treated. The compositions are administered via any of several routes of administration, including topically, orally, parenterally, intravenously, intra-articularly, intraperitoneally, intramuscularly, subcutaneously, intracavity, transdermally, intrahepatically, intracranially, nebulization/inhalation, or by installation via bronchoscopy. In some embodiments, the compositions are administered locally (non-systemically), including intratumorally, intra-articularly, intrathecally, etc.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

Optionally, the chimeric polypeptides or nucleic acid sequences encoding the chimeric polypeptides are administered by a vector. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based delivery systems. Such methods are well known in the art and readily adaptable for use with the compositions and methods described herein. Such compositions and methods can be used to transfect or transduce cells in vitro or in vivo, for example, to produce cell lines that express and preferably secrete the encoded chimeric polypeptide or to therapeutically deliver nucleic acids to a subject. The components of the chimeric nucleic acids disclosed herein typically are operably linked in frame to encode a fusion protein.

As used herein, plasmid or viral vectors are agents that transport the disclosed nucleic acids into the cell without degradation and include a promoter yielding expression of the nucleic acid molecule and/or polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., Retroviruses, Cold Spring Harbor Laboratory Press (1997), which is incorporated by reference herein for the vectors and methods of making them. The construction of replication-defective adenoviruses has been described (Berkner et al., J. Virol. 61:1213-20 (1987); Massie et al., Mol. Cell. Biol. 6:2872-83 (1986); Haj-Ahmad et al., J. Virol. 57:267-74 (1986); Davidson et al., J. Virol. 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infectious viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided polypeptides and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004).

The provided polypeptides can be delivered by subviral dense bodies (DBs). DBs transport proteins into target cells by membrane fusion. Methods for making and using DBs are described in, for example, Pepperl-Klindworth et al., Gene Therapy 10:278-84 (2003).

The provided polypeptides can be delivered by tegument aggregates. Methods for making and using tegument aggregates are described in International Publication No. WO 2006/110728.

Non-viral based delivery methods, can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Pal Alto, Calif), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns. Such vectors can also be used to make the chimeric polypeptides by expression is a suitable host cell, such as CHO cells.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g. β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g. chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion with the encoded polypeptide. Such tags can be inserted anywhere within the polypeptide including at either the carboxyl or amino terminus.

As used herein, the terms peptide, polypeptide, or protein are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more. As used throughout, subject can be a vertebrate, more specifically a mammal (e.g. a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a disease or disorder (e.g. cancer). The term patient or subject includes human and veterinary subjects.

A subject at risk of developing a disease or disorder can be genetically predisposed to the disease or disorder, e.g., have a family history or have a mutation in a gene that causes the disease or disorder, or show early signs or symptoms of the disease or disorder. A subject currently with a disease or disorder has one or more than one symptom of the disease or disorder and may have been diagnosed with the disease or disorder.

The methods and agents as described herein are useful for both prophylactic and therapeutic treatment. For prophylactic use, a therapeutically effective amount of the chimeric polypeptides or chimeric nucleic acid sequences encoding the chimeric polypeptides described herein are administered to a subject prior to onset (e.g., before obvious signs of cancer or inflammation) or during early onset (e.g., upon initial signs and symptoms of cancer or inflammation). Prophylactic administration can occur for several days to years prior to the manifestation of symptoms of cancer or inflammation. Prophylactic administration can be used, for example, in the preventative treatment of subjects diagnosed with a genetic predisposition to cancer. Therapeutic treatment involves administering to a subject a therapeutically effective amount of the chimeric polypeptides or nucleic acid sequences encoding the chimeric polypeptides described herein after diagnosis or development of cancer or inflammation (e.g., an autoimmune disease). Prophylactic use may also apply when a patient is undergoing a treatment, e.g., a chemotherapy, in which inflammation is expected.

According to the methods taught herein, the subject is administered an effective amount of the agent (e.g., a chimeric polypeptide). The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products.

As used herein the terms treatment, treat, or treating refers to a method of reducing the effects of a disease or condition or symptom of the disease or condition. Thus, in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refers to an action, for example, administration of the chimeric polypeptide or nucleic acid sequence encoding the chimeric polypeptide, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

IL-2 variants have been developed that are selective for IL2Rαβfy relative to IL2Rβγ (Shanafelt, A. B., et al., 2000, Nat Biotechnol.18:1197-202; Cassell, D. J., et. al., 2002, Curr Pharm Des., 8:2171-83). These variants have amino acid substitutions which reduce their affinity for IL2RB. Because IL-2 has undetectable affinity for IL2RG, these variants consequently have reduced affinity for the IL2Rβγ receptor complex and reduced ability to activate IL2Rβγ-expressing cells, but retain the ability to bind IL2RA and the ability to bind and activate the IL2Rαβry receptor complex.

One of these variants, IL2/N88R (Bay 50-4798), was clinically tested as a low-toxicity version of IL-2 as an immune system stimulator, based on the hypothesis that IL2Rβγ-expressing NK cells are a major contributor to toxicity. Bay 50-4798 was shown to selectively stimulate the proliferation of activated T cells relative to NK cells, and was evaluated in phase I/II clinical trials in cancer patients (Margolin, K., et. al., 2007, Clin Cancer Res., 13:3312-9) and HIV patients (Davey, R. T., et. al., 2008, J Interferon Cytokine Res., 28:89-100). These clinical trials showed that Bay 50-4798 was considerably safer and more tolerable than aldesleukin, and also showed that it increased the levels of CD4+CD25+ T cells, a cell population enriched in Treg cells. Subsequent to these trials, research in the field more fully established the identity of Treg cells and demonstrated that Treg cells selectively express IL2Rαβγ (reviewed in Malek, T. R., et al., 2010, Immunity, 33:153-65).

In addition, mutants can be made that selectively alter the affinity for the CD25 chain relative to native 11-2.

IL-2 can be engineered to produce mutants that bind the IL-2R complex generally or the IL-2Rα subunit specifically with an affinity that differs from that of the corresponding wild-type IL-2 or of a presently available mutant (referred to as C125S, as the cysteine residue at position 125 is replaced with a serine residue).

Accordingly, the present invention features mutant interleukin-2 (IL-2*) polypeptides that include an amino acid sequence that is at least 80% identical to wild-type IL-2 (e.g., 85, 87, 90, 95, 97, 98, or 99% identical) and that bind, as compared to WT IL-2, with higher to the IL-2 trimeric receptor relative to the dimeric IL-2 receptor. Typically, the muteins will also bind an IL-2 receptor a subunit (IL-2Rα) with an affinity that is greater than the affinity with which wild type IL-2 binds the IL-2Rα. The amino acid sequence within mutant IL-2 polypeptides can vary from SEQ ID NO:1 (UniProtKB accession number P60568) by virtue of containing (or only containing) one or more amino acid substitutions, which may be considered conservative or non-conservative substitutions. Non-naturally occurring amino acids can also be incorporated. Alternatively, or in addition, the amino acid sequence can vary from SEQ ID NO:1 (which may be considered the “reference” sequence) by virtue of containing and addition and/or deletion of one or more amino acid residues. More specifically, the amino acid sequence can differ from that of SEQ ID NO:1 by virtue of a mutation at least one of the following positions of SEQ ID NO:1: 1, 4, 8, 9, 10, 11, 13, 15, 26, 29, 30, 31, 35, 37, 46, 48, 49, 54, 61, 64, 67, 68, 69, 71, 73, 74, 75, 76, 79, 88, 89, 90, 92, 99, 101, 103, 114, 125, 128, or 133 (or combinations thereof). As noted, as few as one of these positions may be altered, as may two, three, four, five, six, seven, eight, nine, ten, or 11 or more (including up to all) of the positions. For example, the amino acid sequence can differ from SEQ ID NO:1 at positions 69 and 74 and further at one or more of positions 30, 35, and 128. The amino acid sequence can also differ from SEQ ID NO:2 (as disclosed in U.S. Pat. No. 7,569,215, incorporated herein by reference) at one of the following sets of positions: (a) positions 64, 69, and 74; (b) positions 69, 74, and 101; (c) positions 69, 74, and 128; (d) positions 30, 69, 74, and 103; (e) positions 49, 69, 73, and 76; (f) positions 69, 74, 101, and 133; (g) positions 30, 69, 74, and 128; (h) positions 69, 74, 88, and 99; (i) positions 30, 69, 74, and 128; (j) positions 9, 11, 35, 69, and 74; (k) positions 1, 46, 49, 61, 69, and 79; (1) positions 48, 68, 71, 90, 103, and 114; (m) positions 4, 10, 11, 69, 74, 88, and 133; (n) positions 15, 30 31, 35, 48, 69, 74, and 92; (O) positions 30, 68, 69, 71, 74, 75, 76, and 90; (p) positions 30, 31, 37, 69, 73, 74, 79, and 128; (q) positions 26, 29, 30, 54, 67, 69, 74, and 92; (r) positions 8, 13, 26, 30, 35, 37, 69, 74, and 92; and (s) positions 29, 31, 35, 37, 48, 69, 71, 74, 88, and 89. Aside from mutations at these positions, the amino acid sequence of the mutant IL-2 polypeptide can otherwise be identical to SEQ ID NO:1. With respect to specific substitutions, the amino acid sequence can differ from SEQ ID NO:1 by virtue of having one or more of the following mutations: AlT, S4P, K8R, K9T, T10A, Q11R, Q13R, E15K, N26D, N29S, N30S, N30D, N30T, Y31H, Y31C, K35R, T37A, T37R, M46L, K48E, K49R, K49E, K54R, E61D, K64R, E67G, E68D, V69A, N71T, N71A, N71R, A73V, Q74P, S75P, K76E, K76R, H79R, N88D, I89V, N90H, I92T, S99P, T101A, F103S, I114V, I128T, I128A, T133A, or T133N. Our nomenclature is consistent with that of the scientific literature, where the single letter code of the amino acid in the wild-type or reference sequence is followed by its position within the sequence and then by the single letter code of the amino acid with which it is replaced. Thus, AlT designates a substitution of the alanine residue a position 1 with threonine. Other mutant polypeptides within the scope of the invention include those that include a mutant of SEQ ID NO:2 having substitutions at V69 (e.g. A) and Q74 (e.g., P). For example, the amino acid sequence can include one of the following sets of mutations with respect to SEQ ID NO:2: (a) K64R, V69A, and Q74P; (b) V69A, Q74P, and T101A; (c) V69A, Q74P, and I128T; (d) N30D, V69A, Q74P, and F103S; (e) K49E, V69A, A73V, and K76E; (f) V69A, Q74P, T101A, and T133N; (g) N30S, V69A, Q74P, and I128A; (h) V69A, Q74P, N88D, and S99P; (i) N30S, V69A, Q74P, and I128T; j) K9T, Q11R, K35R, V69A, and Q74P; (k) AlT, M46L, K49R, E61D, V69A, and H79R; (1) K48E, E68D, N71T, N90H, F103S, and I114V; (m) S4P, T10A, Q11R, V69A, Q74P, N88D, and T133A; (n) E15K, N30S Y31H, K35R, K48E, V69A, Q74P, and I92T; (o) N30S, E68D, V69A, N71A, Q74P, S75P, K76R, and N90H; (p) N30S, Y31C, T37A, V69A, A73V, Q74P, H79R, and I128T; (q) N26D, N29S, N30S, K54R, E67G, V69A, Q74P, and I92T; (r) K8R, Q13R, N26D, N30T, K35R, T37R, V69A, Q74P, and 192T; and (s) N29S, Y31H, K35R, T37A, K48E, V69A, N71R, Q74P, N88D, and 189V. SEQ ID NO:2 is disclosed in U.S. Pat. No. 7,569,215, which is incorporated herein by reference as an exemplary IL-2 polypeptide sequence that can be used in the invention.

As noted above, any of the mutant IL-2 polypeptides disclosed herein can include the sequences described; they can also be limited to the sequences described and otherwise identical to SEQ ID NO:1. Moreover, any of the mutant IL-2 polypeptides described herein can optionally include a substitution of the cysteine residue at position 125 with another residue (e.g., serine) and/or can optionally include a deletion of the alanine residue at position 1 of SEQ ID NO:1.

The mutant IL-2 polypeptides disclosed herein can bind to the IL-2Rα subunit with a K_{d} of less than about 28 nM (e.g., less than about 25 nM; less than about 5 nM; about 1 nM; less than about 500 pM; or less than about 100 pM). More specifically, a mutant IL-2 polypeptide can have an affinity equilibrium constant less than 1.0 nM (e.g., about 0.8, 0.6, 0.4, or 0.2 nM). Affinity can also be expressed as a relative rate of dissociation from an IL-2Rα subunit or from an IL-2 receptor complex (e.g., a complex expressed on the surface of a cell or otherwise membrane bound). For example, the mutant IL-2 polypeptides can dissociate from, e.g., IL-2Rα, at a decreased rate relative to a wild-type polypeptide or to an IL-2 based therapeutic, e.g., IL-2*. Alternatively, affinity can be characterized as the time, or average time, an IL-2* polypeptide persists on, for example, the surface of a cell expressing an IL-2R. For example, an IL-2*polypeptide can persist on the receptor for at least about 2, 5, 10, 50, 100, or 250 times (or more).

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

The following are examples of methods and compositions of the invention. It is understood that various other embodiments may be practiced, given the general description provided herein.

### Example 1: Detection of IL-2, IL-2 mutein, IL-2Rα and IL-2Rγ in Fusion Proteins by ELISA

IL-2 mutein is detected with a commercially available antibody, e.g., the anti-IL-2 monoclonal (JES6-1A12) (BD Pharmingen; San Jose, Calif.). A positive control is used to show whether the monoclonal antibody recognizes the cytokine or mutein. Antibodies against IL-2Rα and IL-2Rγ chain are also used. Wells of a 96-well plate are coated with an antibody (2.5 g/ml) in PBS. Wells are blocked with 5% non-fat milk in PBS with 0.2% Tween®20 (PBS-M-Tw) and fusion proteins are added for 1-2 hours at 37° C. After washing, an anti-IL-2 biotin-labeled antibody, e.g., JES5H4 (BD Pharmingen) is added and binding is detected using Strepavidin HRP (Southern Biotechnology Associates; Birmingham, Ala.). The ELISA plate is developed by adding 50 μl O-phenylenediamine (OPD) (Sigma-Aldrich) in 0.1M Citrate pH 4.5 and 0.04% H₂O₂, stopped by adding 50 μl/well 2N H₂SO₄and the absorbance was read at 490 n.

### Example 2: Protease Cleavage of Fusion Protein by MMP9 Protease

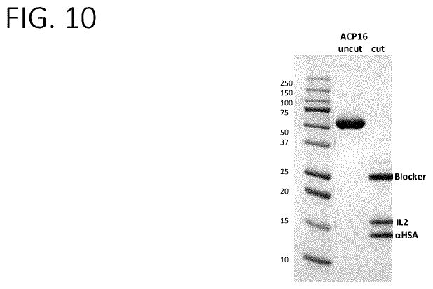

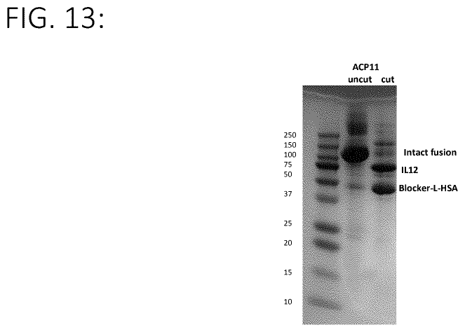

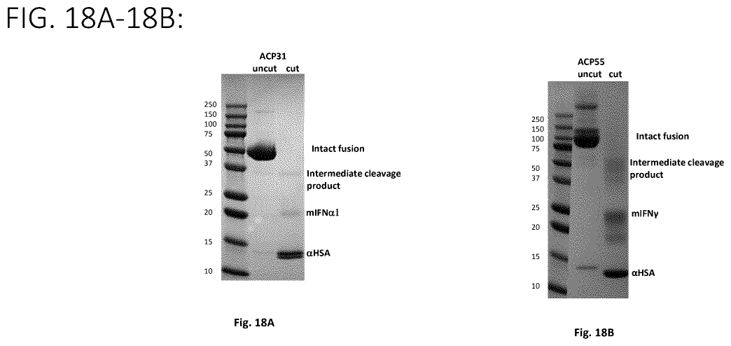

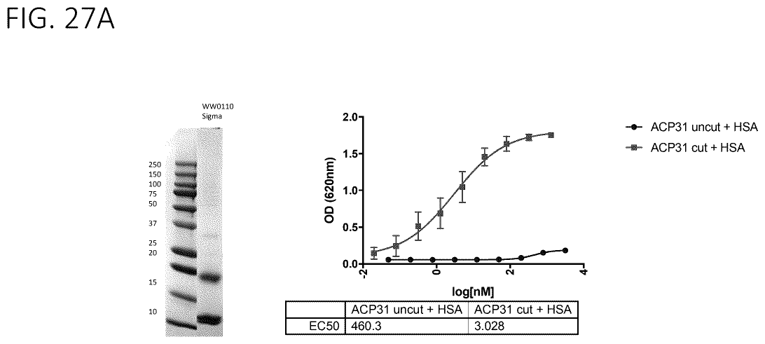

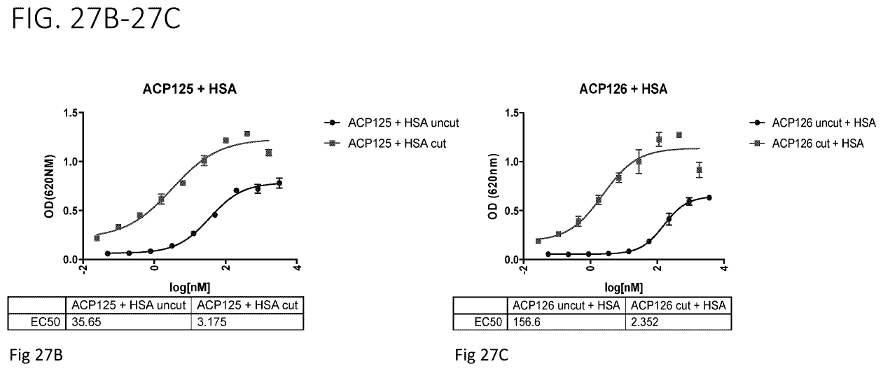

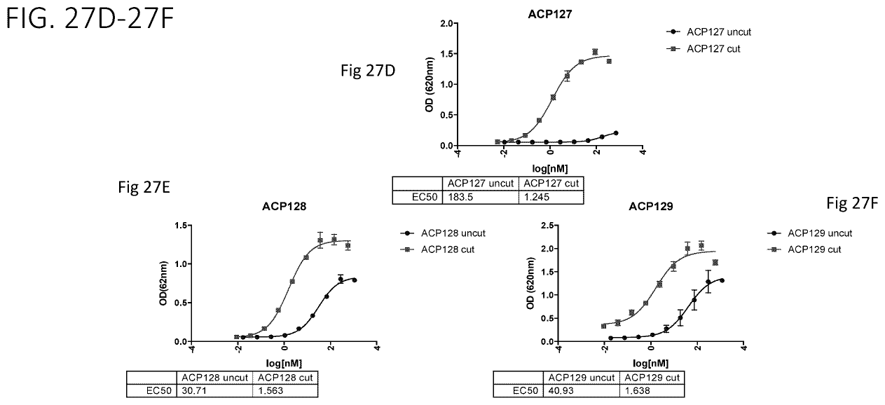

One of skill in the art would be familiar with methods of setting up protein cleavage assay. 100ug of protein in 1×PBS pH 7.4 were cleaved with 1 gg active MMP9 (Sigma catalog #SAE0078-50 or Enzo catalog BML-SE360) and incubated at room temperature for up to 16 hours. Digested protein is subsequently used in functional assays or stored at −80° C. prior to testing. Extent of cleavage was monitored by SDS PAGE using methods well known in the art. As shown in FIGS. 10, 13, 18A, 18B, 24B, 24C, and 27A full cleavage of the fusion proteins by MMP9 protease is seen.

### Example 3: CTLL-2 Assay

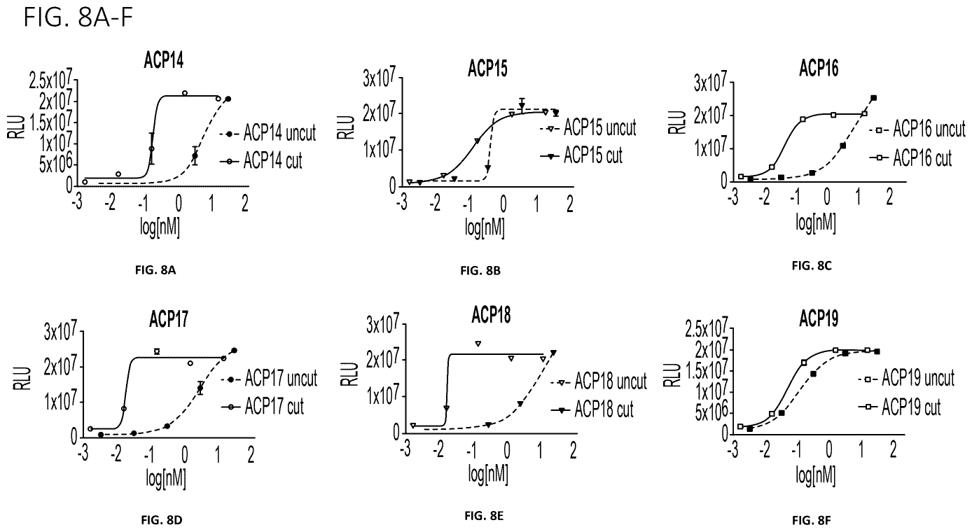

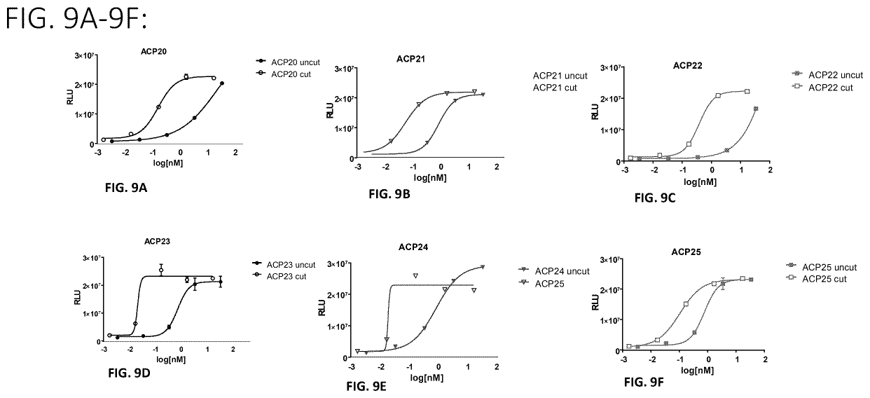

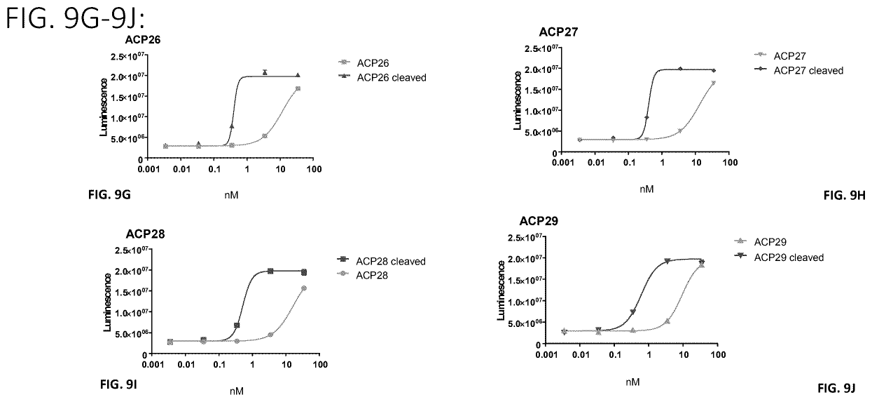

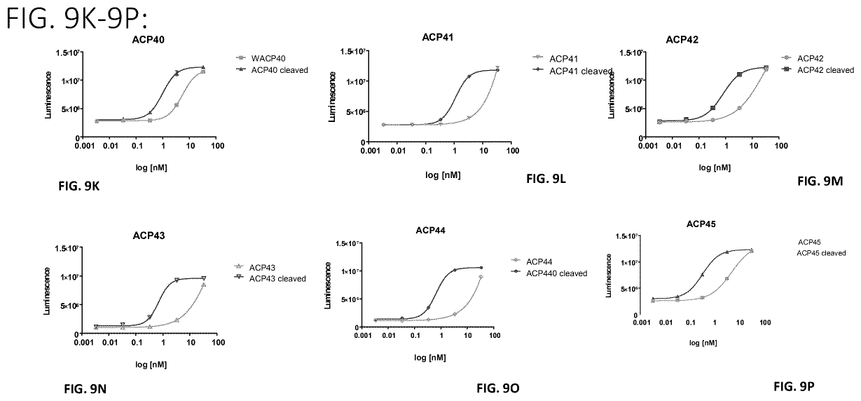

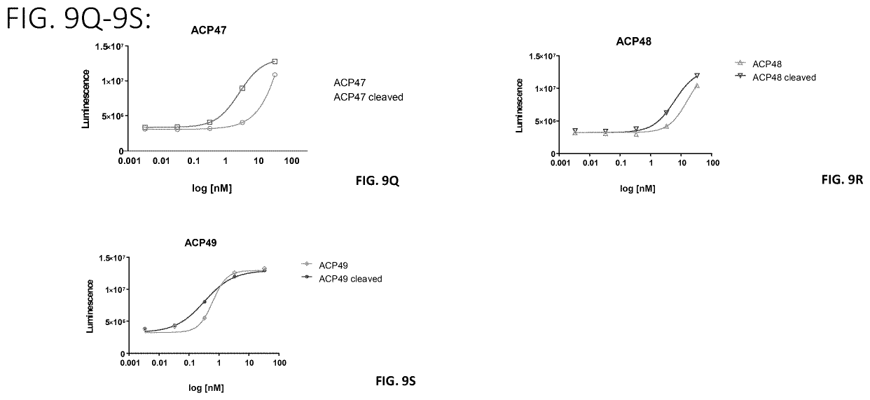

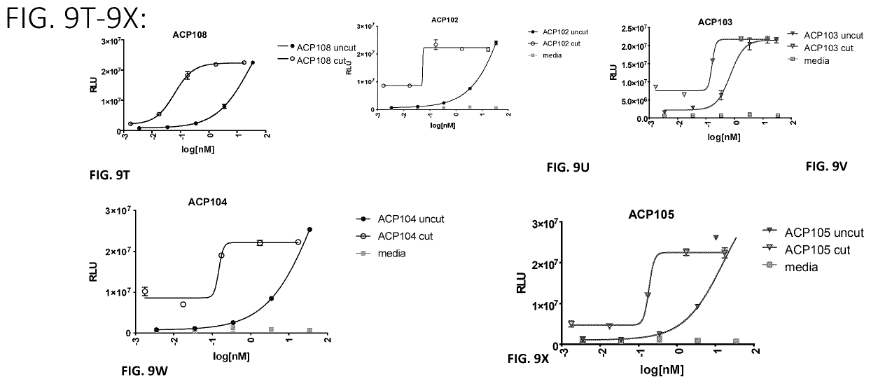

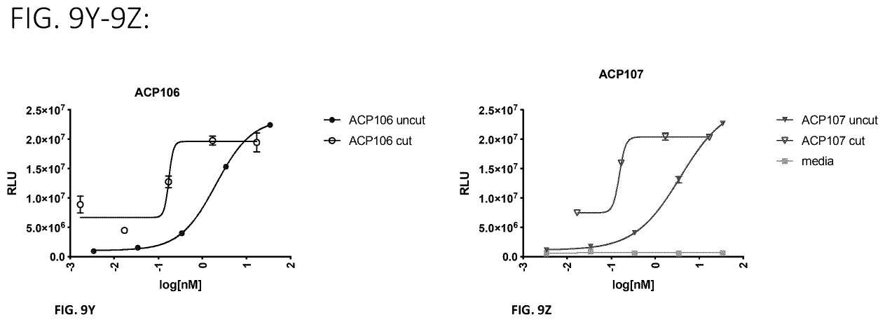

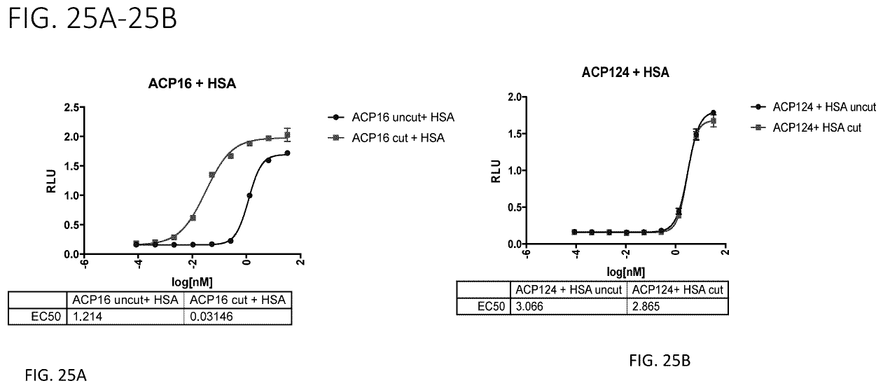

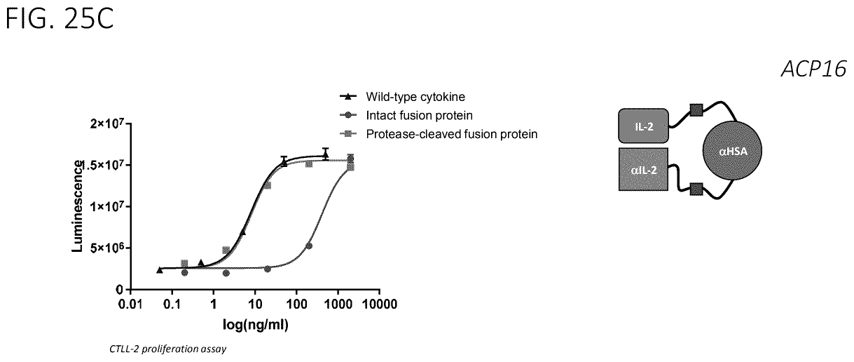

CTLL2 cells (ATCC) were plated in suspension at a concentration of 500,000 cells/well in culture media with or without 40 mg/ml human serum albumin (HSA) and stimulated with a dilution series of recombinant hIL2 or activatable hIL2 for 72 hours at 37° C. and 5% CO₂. Activity of uncleaved and cleaved activatable hIL2 was tested. Cleaved activatable hIL2 was generated by incubation with active MMP9. Cell activity was assessed using a CellTiter-Glo (Promega) luminescence-based cell viability assay. Results are shown in FIGS. 8, 9, and 25.

### Example 4: Protease Cleavage of the IL-2/IL-2Ri/IL-2Rγ Chimeric Polypeptide Results in Increased Accessibility to Antibodies and Biologically Active IL-2 Mutein

The IL-2 mutein fusion proteins are biochemically characterized before and after cleavage with a protease, e.g., PSA. Immunoblot analyses will show that the fusion proteins can be cleaved by PSA and that there is an increase in intensity of the predicted low molecular weight cleavage product of approximately 20 kDa reactive with an anti-IL-2 antibody after treatment of the samples with PSA. The degree of cleavage is dependent upon the amount of PSA as well as the time of incubation. Interestingly, when the fusion protein is analyzed before and after PSA treatment by ELISA, it was found that the apparent amount of IL-2 is increased after PSA cleavage. In this experiment, there is an approximately 2 or 4-fold increase in the apparent amount of IL-2 detected using this sandwich ELISA depending on the construct, suggesting that the antibody binding is partially hindered in the intact fusion protein. Aliquots of the same samples are also analyzed after PSA treatment using the CTLL-2 cell line that requires IL-2 for growth and survival and the viability of cells can be ascertained using the colorimetric MTT assay. In this assay, the more a supernatant can be diluted, the more biologically active IL-2 it contains, and there is an increase in the amount of biologically active IL-2 after PSA cleavage. The amount of IL-2 mutein increase will suggest that after PSA cleavage there is an increase in the predicted low molecular weight cleavage fragment of approximately 20 kDa reactive with an anti-IL-2 antibody, an increase in antibody accessibility, and most importantly, an increase in the amount of biologically active IL-2 mutein.

### Example 5. In Vivo Delivery of a Protease Activated Fusion Protein Results in Decreased Tumor Growth

The chimeric polypeptide is examined to determine if it could have biological effects in vivo. For these experiments a system is used in which tumor cells injected intraperitoneally rapidly and preferentially attach and grow initially on the milky spots, a series of organized immune aggregates found on the omentum (Gerber et al., Am. J. Pathol. 169:1739-52 (2006)). This system offers a convenient way to examine the effects of fusion protein treatment on tumor growth since fusion proteins can be delivered intraperitoneally multiple times and tumor growth can be analyzed by examining the dissociated omental cells. For these experiments, the Colon 38 cell line, a rapidly growing tumor cell line that expresses both MMP2 and MMP9 in vitro, may be used. The omental tissue normally expresses a relatively small amount of MMP2 and MMP9, but, when Colon 38 tumor is present on the omentum, MMP levels increase. Using this tumor model, the ability of IL-2 mutein fusion proteins to affect tumor growth is examined. Colon 38 cells are injected intraperitoneally, allowed to attach and grow for 1 day, and then treated daily with fusion protein interaperitoneally. At day 7, the animals are sacrificed and the omenta examined for tumor growth using flow cytometry and by a colony-forming assay.

### Example 6: Construction of an Exemplary Activatable IL2 Protein Targeting CD20

### Generation of an Activatable IL2 Domain

An IL-2 polypeptide capable of binding to CD20 polypeptide present in a tumor or on a tumor cell is produced as follows. A nucleic acid is produced that contains nucleic acid sequences: (1) encoding an IFNγ polypeptide sequence and (2) one or more polypeptide linkers. Activatable interleukin plasmid constructs can have optional Flag, His or other affinity tags, and are electroporated into HEK293 or other suitable human or mammalian cell lines and purified. Validation assays include T cell activation assays using T cells responsive to IFNγ stimulation in the presence of a protease.

Generation of a scFv CD20 Binding Domain

CD20 is one of the cell surface proteins present on B-lymphocytes. CD20 antigen is found in normal and malignant pre-B and mature B lymphocytes, including those in over 90% of B-cell non-Hodgkin's lymphomas (NHL). The antigen is absent in hematopoietic stem cells, activated B lymphocytes (plasma cells) and normal tissue. As such, several antibodies mostly of murine origin have been described: 1F5, 2B8/C2B8, 2H7, and 1H4.

Human or humanized anti-CD20 antibodies are therefore used to generate scFv sequences for CD20 binding domains of an activatable interleukin protein. DNA sequences coding for human or humanized VL and VH domains are obtained, and the codons for the constructs are, optionally, optimized for expression in cells from Homo sapiens. The order in which the VL and VH domains appear in the scFv is varied (i.e., VL-VH, or VH-VL orientation), and three copies of the “G4S” (SEQ ID NO.: 449) or “G₄S” (SEQ ID NO.: 449) subunit (G₄S)₃ (SEQ ID NO.: 452) connect the variable domains to create the scFv domain. Anti-CD20 scFv plasmid constructs can have optional Flag, His or other affinity tags, and are electroporated into HEK293 or other suitable human or mammalian cell lines and purified. Validation assays include binding analysis by FACS, kinetic analysis using Proteon, and staining of CD20-expressing cells.

### Cloning of DNA Expression Constructs Encoding the Activatable IL2 Protein

The activatable IL2 construct with protease cleavage site domains are used to construct an activatable interleukin protein in combination with an anti-CD20 scFv domain and a serum half-life extension element (e.g., a HSA binding peptide or VH domain). For expression of an activatable interleukin protein in CHO cells, coding sequences of all protein domains are cloned into a mammalian expression vector system. In brief, gene sequences encoding the activatable interleukin domain, serum half-life extension element, and CD20 binding domain along with peptide linkers L1 and L2 are separately synthesized and subcloned. The resulting constructs are then ligated together in the order of CD20 binding domain—L1—IL2 subunit 1-L2-protease cleavage domain -L3-IL2 subunit 2-L4-anti-CD20 scFv -L5-serum half-life extension element to yield a final construct. All expression constructs are designed to contain coding sequences for an N-terminal signal peptide and a C-terminal hexahistidine (6×His)-tag (SEQ ID NO. 354) to facilitate protein secretion and purification, respectively.

### Expression of Activatable IL2 Proteins in Stably Transfected CHO Cells

A CHO cell expression system (Flp-Jn®, Life Technologies), a derivative of CHO-K1 Chinese Hamster ovary cells (ATCC, CCL-61) (Kao and Puck, Proc. Natl. Acad Sci USA 1968; 60(4):1275-81), is used. Adherent cells are subcultured according to standard cell culture protocols provided by Life Technologies.

For adaption to growth in suspension, cells are detached from tissue culture flasks and placed in serum-free medium. Suspension-adapted cells are cryopreserved in medium with 10% DMSO.

Recombinant CHO cell lines stably expressing secreted activatable interleukin proteins are generated by transfection of suspension-adapted cells. During selection with the antibiotic Hygromycin B viable cell densities are measured twice a week, and cells are centrifuged and resuspended in fresh selection medium at a maximal density of 0.1×10⁶ viable cells/mL. Cell pools stably expressing activatable interleukin proteins are recovered after 2-3 weeks of selection at which point cells are transferred to standard culture medium in shake flasks. Expression of recombinant secreted proteins is confirmed by performing protein gel electrophoresis or flow cytometry. Stable cell pools are cryopreserved in DMSO containing medium.

Activatable IL2 proteins are produced in 10-day fed-batch cultures of stably transfected CHO cell lines by secretion into the cell culture supernatant. Cell culture supernatants are harvested after 10 days at culture viabilities of typically >75%. Samples are collected from the production cultures every other day and cell density and viability are assessed. On day of harvest, cell culture supernatants are cleared by centrifugation and vacuum filtration before further use.

Protein expression titers and product integrity in cell culture supernatants are analyzed by SDS-PAGE.

### Purification of Activatable IL2 Proteins

Activatable IL2 proteins are purified from CHO cell culture supernatants in a two-step procedure. The constructs are subjected to affinity chromatography in a first step followed by preparative size exclusion chromatography (SEC) on Superdex 200 in a second step. Samples are buffer-exchanged and concentrated by ultrafiltration to a typical concentration of >1 mg/mL. Purity and homogeneity (typically >90%) of final samples are assessed by SDS PAGE under reducing and non-reducing conditions, followed by immunoblotting using an anti-HSA or anti idiotype antibody as well as by analytical SEC, respectively. Purified proteins are stored at aliquots at −80° C. until use.

### Example 7: Determination of Antigen Affinity by Flow Cytometry

The activatable interleukin proteins of Example 6 are tested for their binding affinities to human CD20⁺ cells and cynomolgus CD20 cells.

CD20 cells are incubated with 100 μL of serial dilutions of the activatable interleukin proteins of Example 1 and at least one protease. After washing three times with FACS buffer the cells are incubated with 0.1 mL of 10 μg/mL mouse monoclonal anti-idiotype antibody in the same buffer for 45 min on ice. After a second washing cycle, the cells are incubated with 0.1 mL of 15 μg/mL FITC-conjugated goat anti-mouse IgG antibodies under the same conditions as before. As a control, cells are incubated with the anti-His IgG followed by the FITC-conjugated goat anti-mouse IgG antibodies without the activatable IL2 proteins. The cells were then washed again and resuspended in 0.2 mL of FACS buffer containing 2 μg/mL propidium iodide (PI) in order to exclude dead cells. The fluorescence of 1×10⁴ living cells is measured using a Beckman-Coulter FC500 MPL flow cytometer using the MXP software (Beckman-Coulter, Krefeld, Germany) or a Millipore Guava EasyCyte flow cytometer using the Incyte software (Merck Millipore, Schwalbach, Germany). Mean fluorescence intensities of the cell samples are calculated using CXP software (Beckman-Coulter, Krefeld, Germany) or Incyte software (Merck Millipore, Schwalbach, Germany). After subtracting the fluorescence intensity values of the cells stained with the secondary and tertiary reagents alone the values are then used for calculation of the K_{D} values with the equation for one-site binding (hyperbola) of the GraphPad Prism (version 6.00 for Windows, GraphPad Software, La Jolla California USA).

CD20 binding and crossreactivity are assessed on the human CD20⁺ tumor cell lines. The K_{D}ratio of crossreactivity is calculated using the K_{D} values determined on the CHO cell lines expressing either recombinant human or recombinant cynomolgus antigens.

### Example 8: Cytotoxicity Assay

The activatable interleukin protein of Example 6 is evaluated in vitro on its mediation of immune response to CD20T target cells.

Fluorescence labeled CD20 REC-1 cells (a Mantle cell lymphoma cell line, ATCC CRL-3004) are incubated with isolated PBMC of random donors or CB15 T-cells (standardized T-cell line) as effector cells in the presence of the activatable IL2 protein of Example 5 and at least one protease. After incubation for 4 h at 37° C. in a humidified incubator, the release of the fluorescent dye from the target cells into the supernatant is determined in a spectrofluorimeter. Target cells incubated without the activatable IL2 protein of Example land target cells totally lysed by the addition of saponin at the end of the incubation serve as negative and positive controls, respectively.

Based on the measured remaining living target cells, the percentage of specific cell lysis is calculated according to the following formula: [1-(number of living targets₍ₛₐₘₚₗₑ₎/number of living targets₍ₛₚₒₙₜₐₙₑₒᵤₛ₎]×100%. Sigmoidal dose response curves and EC₅₀ values are calculated by non-linear regression/4-parameter logistic fit using the GraphPad Software. The lysis values obtained for a given antibody concentration are used to calculate sigmoidal dose-response curves by 4 parameter logistic fit analysis using the Prism software.

### Example 9: Pharmacokinetics of Activatable Interleukin Proteins

The activatable interleukin protein of Example 6 is evaluated for half-time elimination in animal studies.

The activatable IL2 protein is administered to cynomolgus monkeys as a 0.5 mg/kg bolus injection into the saphenous vein. Another cynomolgus monkey group receives a comparable IL2 construct in size, but lacking a serum half-life extension element. A third and fourth group receive an IL2 construct with serum half-life extension element and a cytokine with CD20 and serum half-life extension elements respectively, and both comparable in size to the activatable interleukin protein. Each test group consists of 5 monkeys. Serum samples are taken at indicated time points, serially diluted, and the concentration of the proteins is determined using a binding ELISA to CD20.

Pharmacokinetic analysis is performed using the test article plasma concentrations. Group mean plasma data for each test article conforms to a multi-exponential profile when plotted against the time post-dosing. The data are fit by a standard two-compartment model with bolus input and first-order rate constants for distribution and elimination phases. The general equation for the best fit of the data for i.v. administration is: c(t)=A^{-αt}+Be^{-βt}, where c(t) is the plasma concentration at time t, A and B are intercepts on the Y-axis, and α and βare the apparent first-order rate constants for the distribution and elimination phases, respectively. The α-phase is the initial phase of the clearance and reflects distribution of the protein into all extracellular fluid of the animal, whereas the second or β-phase portion of the decay curve represents true plasma clearance. Methods for fitting such equations are well known in the art. For example, A=D/V(α−k21)/(α−β), B=D/V(β−k21)/(α−β), and α and β(for α>β) are roots of the quadratic equation: r²+(k12+k21+k10)r+k21k10=0 using estimated parameters of V=volume of distribution, k10=elimination rate, k12=transfer rate from compartment 1 to compartment 2 and k21=transfer rate from compartment 2 to compartment 1, and D=the administered dose.

Data analysis: Graphs of concentration versus time profiles are made using KaleidaGraph (KaleidaGraph™ V. 3.09 Copyright 1986-1997. Synergy Software. Reading, Pa.). Values reported as less than reportable (LTR) are not included in the PK analysis and are not represented graphically. Pharmacokinetic parameters are determined by compartmental analysis using WinNonlin software (WinNonlin® Professional V. 3.1 WinNonlin™ Copyright 1998-1999. Pharsight Corporation. Mountain View, Calif.). Pharmacokinetic parameters are computed as described in Ritschel W A and Kearns G L, 1999, IN: Handbook Of Basic Pharmacokinetics Including Clinical Applications, 5^{th} edition, American Pharmaceutical Assoc., Washington, D.C.

It is expected that the activatable interleukin protein of Example 5 has improved pharmacokinetic parameters such as an increase in elimination half-time as compared to proteins lacking a serum half-life extension element.

### Example 10: Xenograft Tumor Model

The activatable IL2 protein of Example 6 is evaluated in a xenograft model.

Female immune-deficient NOD/scid mice are sub-lethally irradiated (2 Gy) and subcutaneously inoculated with 4×10⁶ Ramos RA1 cells into the right dorsal flank. When tumors reach 100 to 200 mm³, animals are allocated into 3 treatment groups. Groups 2 and 3 (8 animals each) are intraperitoneally injected with 1.5×10⁷ activated human T-cells. Three days later, animals from Group 3 are subsequently treated with a total of 9 intravenous doses of 50 gg activatable interleukin protein of Example 1 (qdx9d). Groups 1 and 2 are only treated with vehicle. Body weight and tumor volume are determined for 30 days.

It is expected that animals treated with the activatable interleukin protein of Example 5 have a statistically significant delay in tumor growth in comparison to the respective vehicle-treated control group.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 11: Mouse IFNγ WEHI Cell Survival Assay

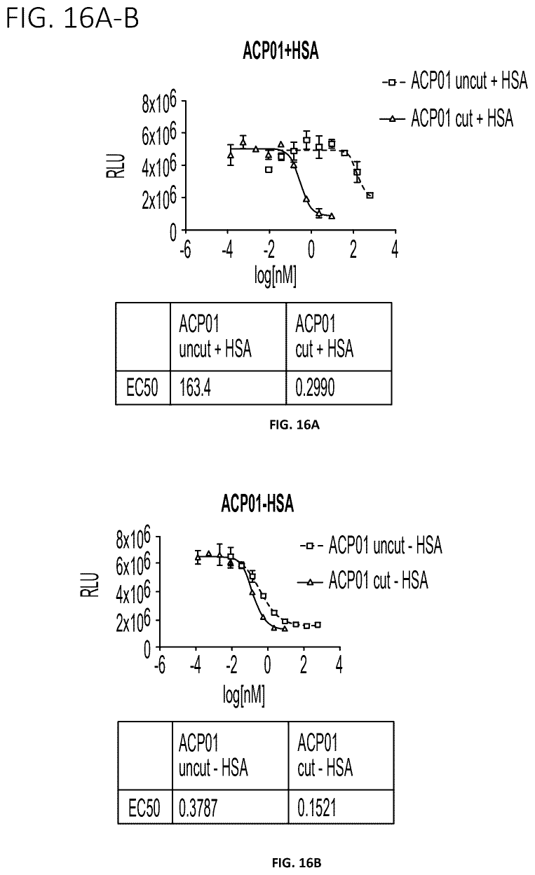

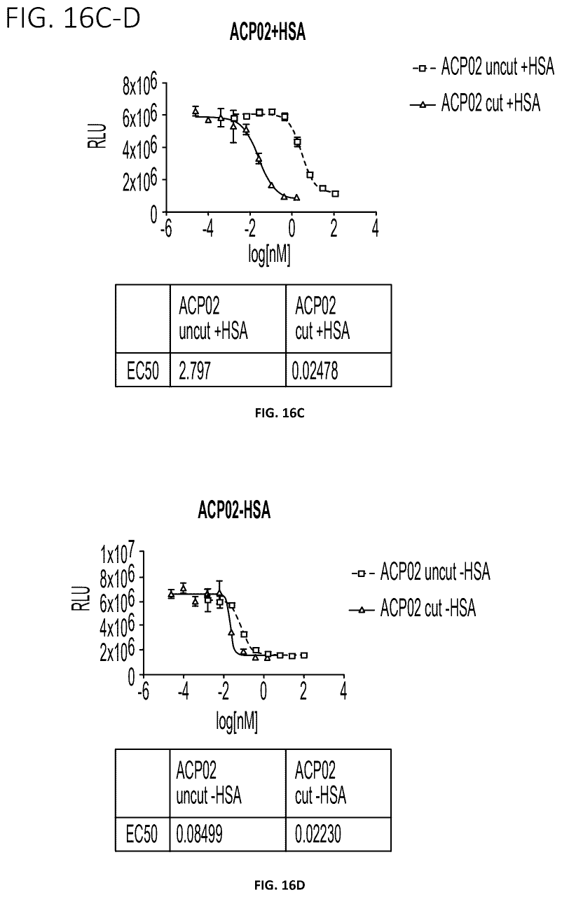

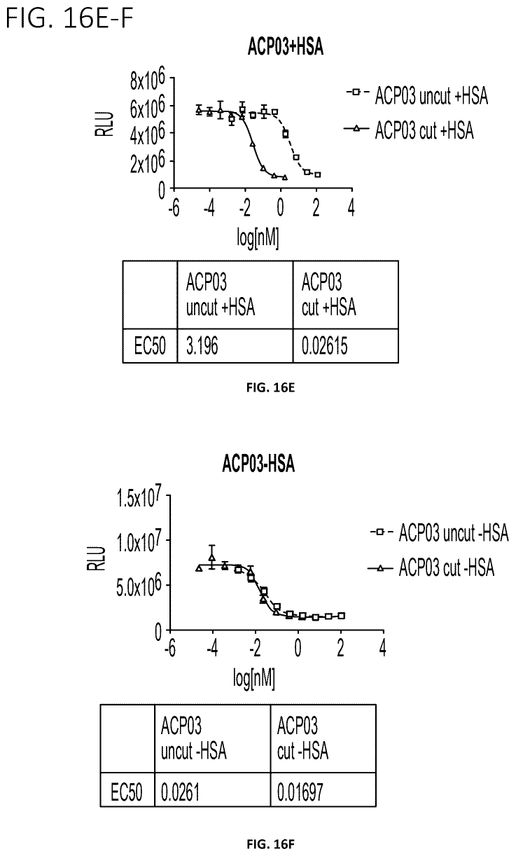

WEHI279 cells (ATCC) were plated in suspension at a concentration of 25,000 cells/well in culture media with or without 1.5% human serum albumin (HSA) and stimulated with a dilution series of recombinant mIFNγ or inducible mIFNγ for 72 hours at 37° C. and 5% CO₂. Activity of uncleaved and cleaved inducible mIFNγ was tested. Cleaved inducible mIFNg was generated by incubation with active MMP9. Cell survival was assessed using a CellTiter-Glo (Promega) luminescence-based cell viability assay. The EC50 values for cleaved inducible mIFNg molecules were at least 100× more potent than un-cleaved inducible mIFNg molecules. As shown in FIGS. 16A-16F, greater inducibility was seen in assays wherein the culture media contained human serum albumin.

### Example 12: Mouse IFNγ B16 reporter and Mouse IFNα/0 B16 reporter cell assays

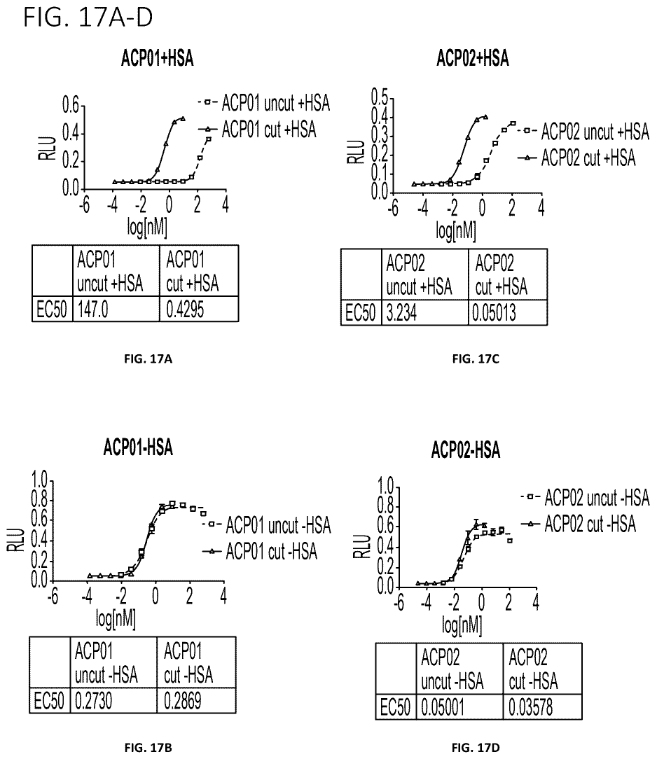

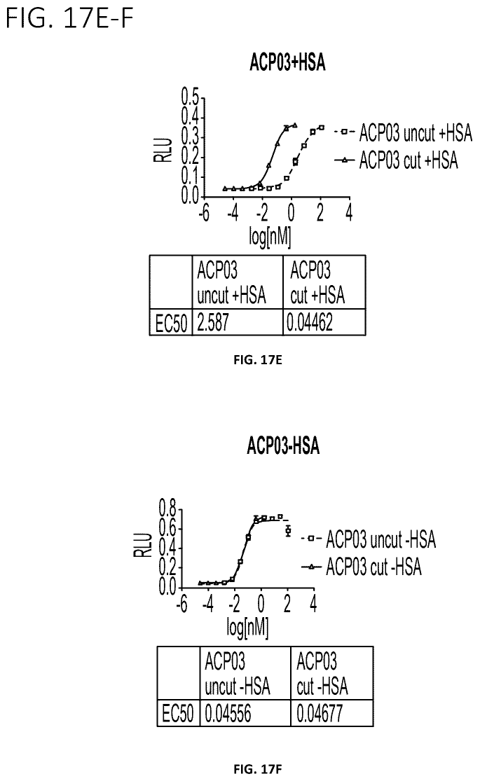

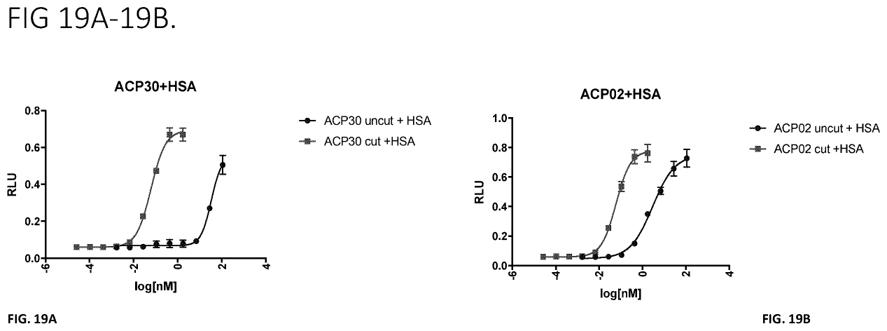

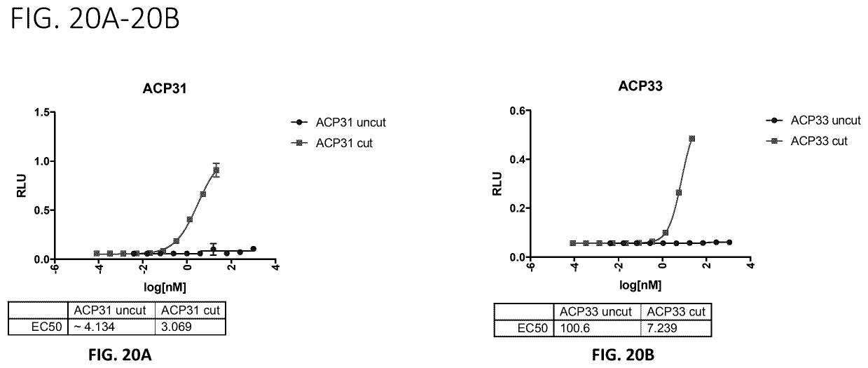

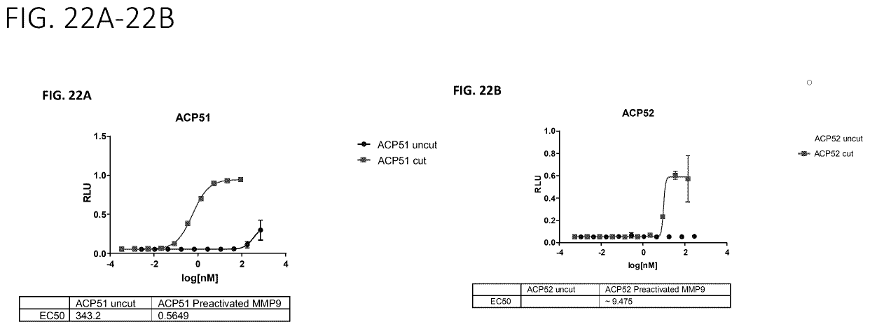

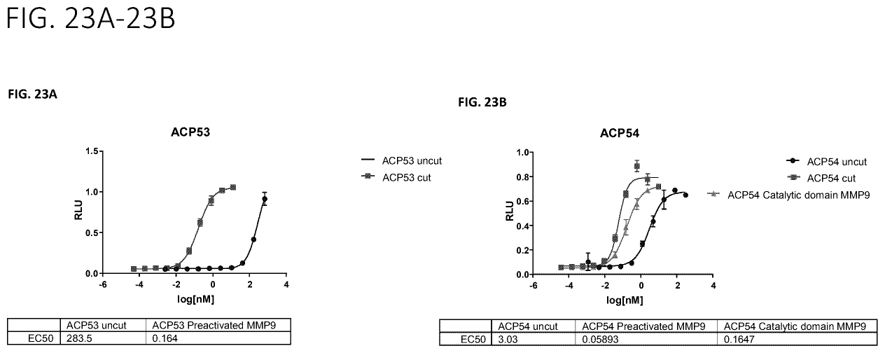

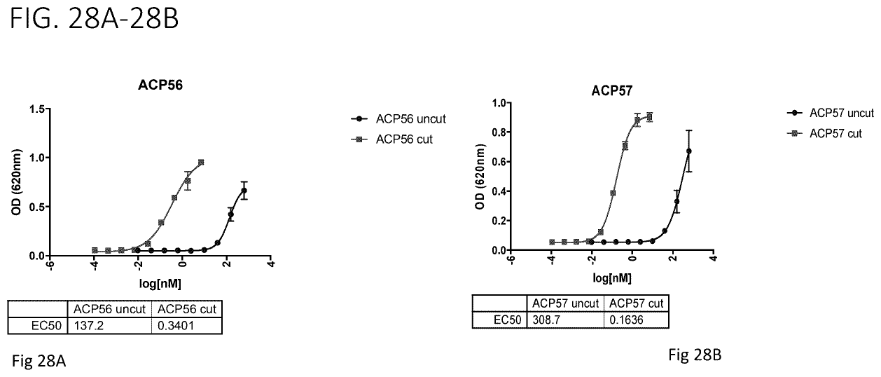

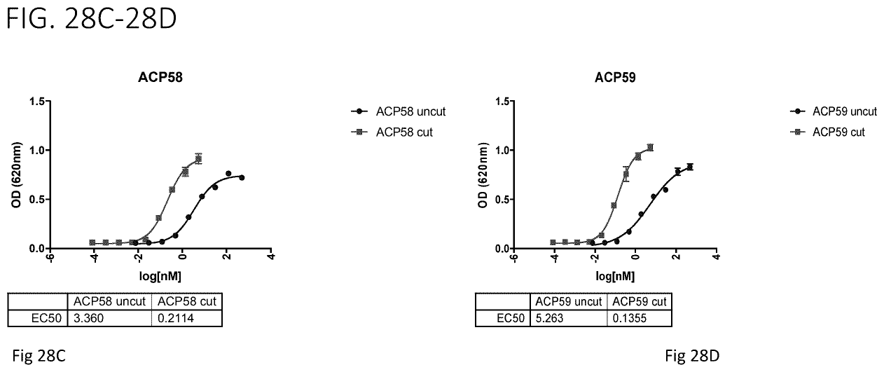

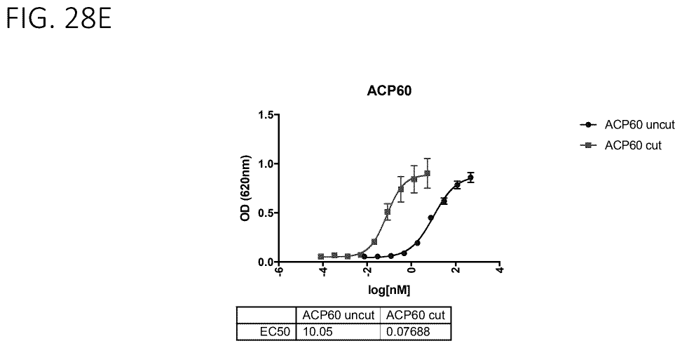

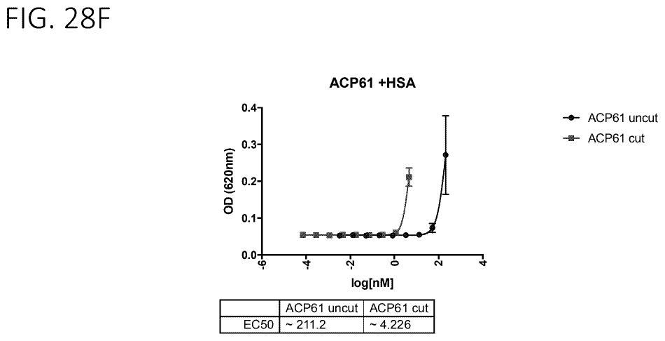

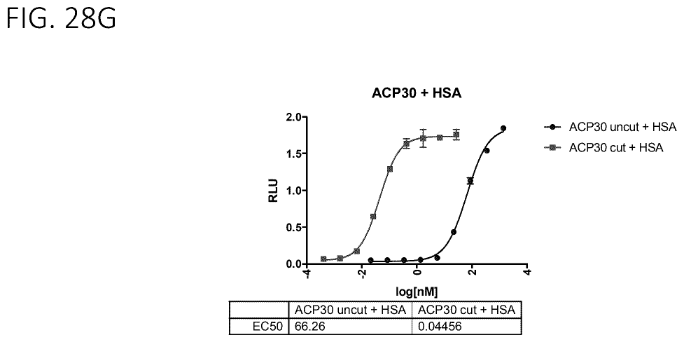

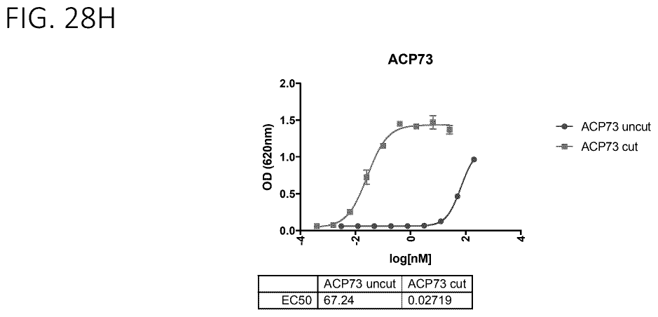

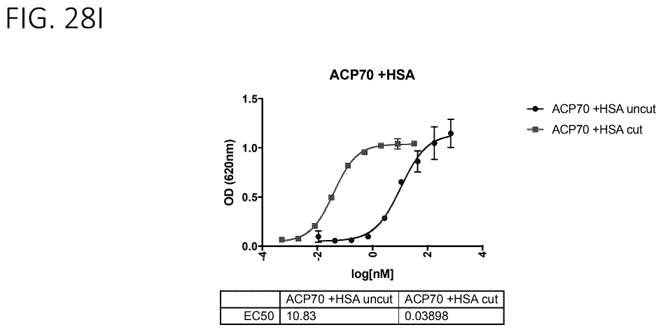

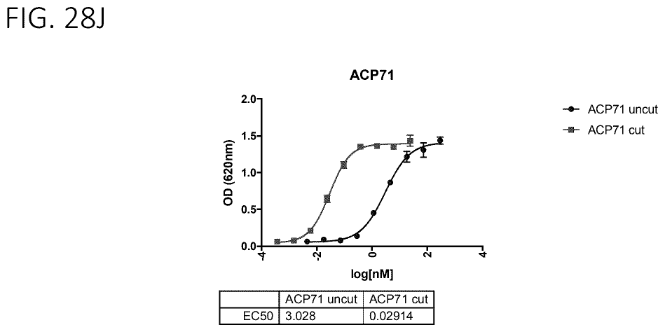

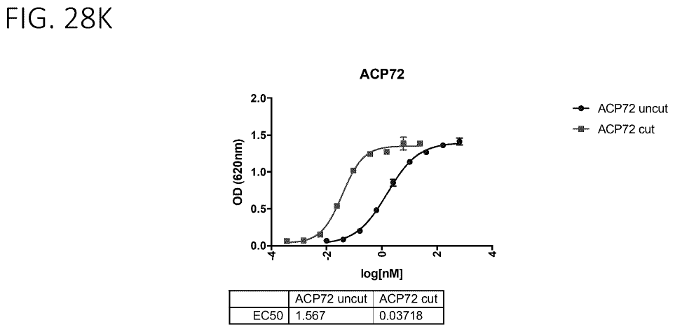

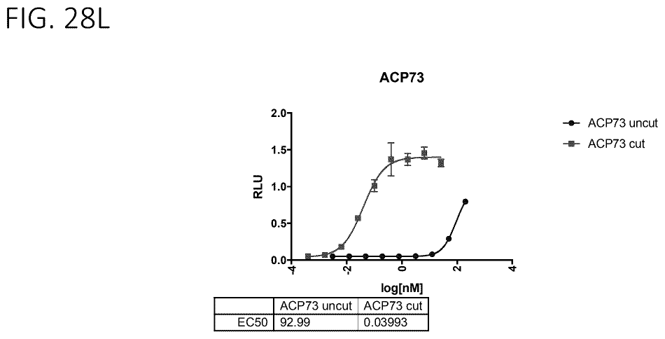

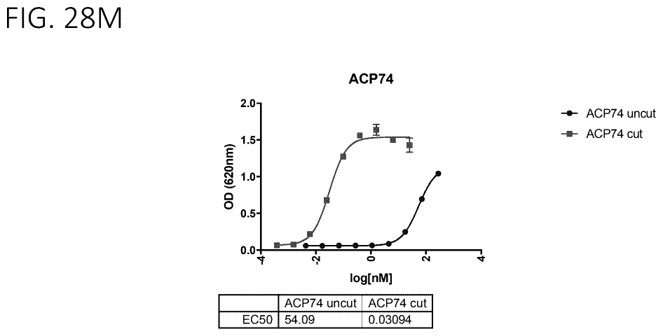

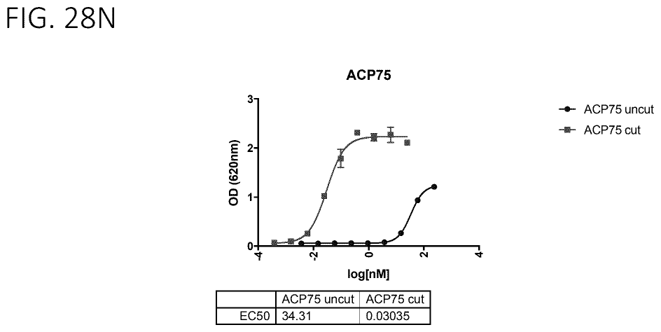

B16-Blue IFNγ cells (InvivoGen) were plated at a concentration of 75,000 cells/well in culture media with or without 1.5% human serum albumin (HSA) and stimulated with a dilution series of recombinant mIFNγ or inducible mIFNγ for 24 hours at 37° C. and 5% CO₂. Activity of uncleaved and cleaved inducible mIFNγ was tested. Cleaved inducible mIFNγ was generated by incubation with active MMP9. Supernatants were harvested, and SEAP activation was assessed by adding QUANTI-Blue Reagent (InvivoGen), incubating at 37° C. for 2 hours, and measuring absorbance at 620 nm. Results are shown in FIGS. 17, 19, 22, 23, and 28. This experiment was repeated with for IFNα fusion proteins using B16-Blue IFNα/β cells. The EC50 values for cleaved inducible mIFNα molecules were at least 100× more potent than un-cleaved inducible mIFNα molecules.

### Example 13. In Vivo Delivery of a Protease Activated Fusion Protein Results in Decreased Tumor Growth

The chimeric polypeptide is examined to determine if it could have biological effects in vivo. For these experiments a system is used in which tumor cells injected intraperitoneally rapidly and preferentially attach and grow initially on the milky spots, a series of organized immune aggregates found on the omentum (Gerber et al., Am. J. Pathol. 169:1739-52 (2006)). This system offers a convenient way to examine the effects of fusion protein treatment on tumor growth since fusion proteins can be delivered intraperitoneally multiple times and tumor growth can be analyzed by examining the dissociated omental cells. For these experiments, the Colon 38 cell line, a rapidly growing tumor cell line that expresses both MMP2 and MMP9 in vitro, may be used. The omental tissue normally expresses a relatively small amount of MMP2 and MMP9, but, when Colon 38 tumor is present on the omentum, MMP levels increase. Using this tumor model, the ability of IFN fusion proteins to affect tumor growth is examined. Colon 38 cells are injected intraperitoneally, allowed to attach and grow for 1 day, and then treated daily with fusion protein interaperitoneally. At day 7, the animals are sacrificed and the omenta examined for tumor growth using flow cytometry and by a colony-forming assay.

### Example 13b: The Chimeric Polypeptide was Examined to Determine its Biological Effects In Vivo

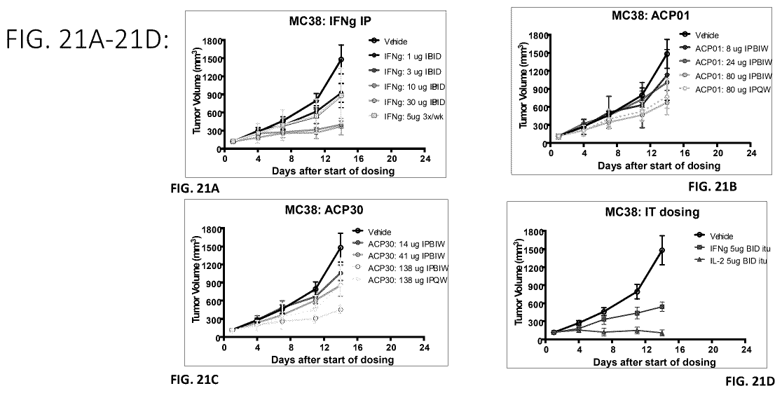

The MC38 cell line, a rapidly growing colon adenocarcinoma cell line that expresses MMP9 in vitro, was used. Using this tumor model, the ability of IFNγ fusion proteins to affect tumor growth was examined. MC38 cells were injected subcutaneously, allowed to grow for 10-14 days, and then treated with fusion protein twice weekly intraperitoneally for a total of four doses, at the levels shown in FIGS. 21A-21D. As a comparator, wild-type mIFNγ was administered at the dose levels indicated, twice daily for 2 weeks on a 5 day on/2 day off schedule (10 total doses). Tumor growth and body weight were monitored approximately twice per week for two weeks.

### Example 14: Construction of an Exemplary IFNγ Protein Targeting CD20

### Generation of an Activatable Cytokine Domain

An IFNγ polypeptide capable of binding to CD20 polypeptide present in a tumor or on a tumor cell is produced as follows. A nucleic acid is produced that contains nucleic acid sequences: (1) encoding an IFNγ polypeptide sequence and (2) one or more polypeptide linkers. Activatable IFNγ plasmid constructs can have optional Flag, His or other affinity tags, and are electroporated into HEK293 or other suitable human or mammalian cell lines and purified. Validation assays include T cell activation assays using T cells responsive to IFNγ stimulation in the presence of a protease.

Generation of a scFv CD20 Binding Domain

CD20 is one of the cell surface proteins present on B-lymphocytes. CD20 antigen is found in normal and malignant pre-B and mature B lymphocytes, including those in over 90% of B-cell non-Hodgkin's lymphomas (NHL). The antigen is absent in hematopoietic stem cells, activated B lymphocytes (plasma cells) and normal tissue. As such, several antibodies mostly of murine origin have been described: 1F5, 2B8/C2B8, 2H7, and 1H4.

Human or humanized anti-CD20 antibodies are therefore used to generate scFv sequences for CD20 binding domains of an activatable IFNγ protein. DNA sequences coding for human or humanized VL and VH domains are obtained, and the codons for the constructs are, optionally, optimized for expression in cells from Homo sapiens. The order in which the VL and VH domains appear in the scFv is varied (i.e., VL-VH, or VH-VL orientation), and three copies of the “G4S” (SEQ ID NO.: 449) or “G₄S” (SEQ ID NO.: 449) subunit (G₄S)₃ (SEQ ID NO.: 452) connect the variable domains to create the scFv domain. Anti-CD20 scFv plasmid constructs can have optional Flag, His or other affinity tags, and are electroporated into HEK293 or other suitable human or mammalian cell lines and purified. Validation assays include binding analysis by FACS, kinetic analysis using Proteon, and staining of CD20-expressing cells.

### Cloning of DNA Expression Constructs Encoding the Activatable IFNγ Protein

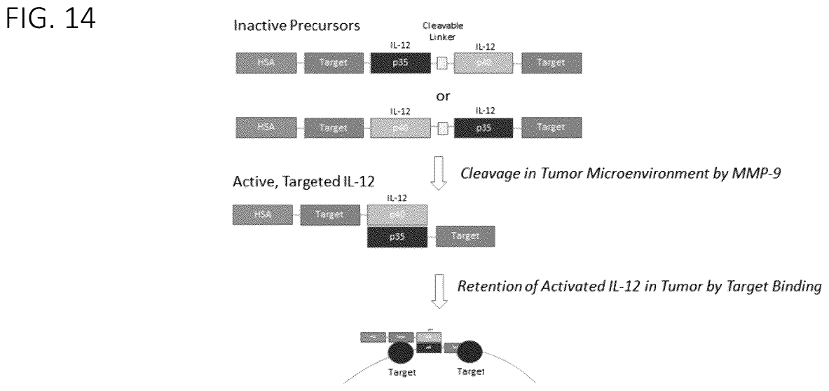

The activatable IFNγ construct with protease cleavage site domains are used to construct an activatable IFNγ protein in combination with an anti-CD20 scFv domain and a serum half-life extension element (e.g., a HSA binding peptide or VH domain), with the domains organized as shown in FIG. 14. For expression of an activatable IFNγ protein in CHO cells, coding sequences of all protein domains are cloned into a mammalian expression vector system. In brief, gene sequences encoding the activatable IFNγ domain, serum half-life extension element, and CD20 binding domain along with peptide linkers L1 and L2 are separately synthesized and subcloned. The resulting constructs are then ligated together in the order of CD20 binding domain -L1-IFNγ subunit 1-L2-protease cleavage domain -L3-IFNγ subunit2-L4-anti-CD20 scFv -L5-serum half-life extension element to yield a final construct. All expression constructs are designed to contain coding sequences for an N-terminal signal peptide and a C-terminal hexahistidine (6×His)-tag (SEQ ID NO.: 354) to facilitate protein secretion and purification, respectively.

### Expression of Activatable IFNγ Proteins in Stably Transfected CHO Cells

A CHO cell expression system (Flp-In®, Life Technologies), a derivative of CHO-K1 Chinese Hamster ovary cells (ATCC, CCL-61) (Kao and Puck, Proc. Natl. Acad Sci USA 1968; 60(4):1275-81), is used. Adherent cells are subcultured according to standard cell culture protocols provided by Life Technologies.

For adaption to growth in suspension, cells are detached from tissue culture flasks and placed in serum-free medium. Suspension-adapted cells are cryopreserved in medium with 10% DMSO.

Recombinant CHO cell lines stably expressing secreted activatable IFNγ proteins are generated by transfection of suspension-adapted cells. During selection with the antibiotic Hygromycin B viable cell densities are measured twice a week, and cells are centrifuged and resuspended in fresh selection medium at a maximal density of 0.1×10⁶ viable cells/mL. Cell pools stably expressing activatable IFNγ proteins are recovered after 2-3 weeks of selection at which point cells are transferred to standard culture medium in shake flasks. Expression of recombinant secreted proteins is confirmed by performing protein gel electrophoresis or flow cytometry. Stable cell pools are cryopreserved in DMSO containing medium.

Activatable IFNγ proteins are produced in 10-day fed-batch cultures of stably transfected CHO cell lines by secretion into the cell culture supernatant. Cell culture supernatants are harvested after 10 days at culture viabilities of typically >75%. Samples are collected from the production cultures every other day and cell density and viability are assessed. On day of harvest, cell culture supernatants are cleared by centrifugation and vacuum filtration before further use.

Protein expression titers and product integrity in cell culture supernatants are analyzed by SDS-PAGE.

### Purification of Activatable IFNγ Proteins

Activatable IFNγ proteins are purified from CHO cell culture supernatants in a two-step procedure. The constructs are subjected to affinity chromatography in a first step followed by preparative size exclusion chromatography (SEC) on Superdex 200 in a second step. Samples are buffer-exchanged and concentrated by ultrafiltration to a typical concentration of >1 mg/mL. Purity and homogeneity (typically >90%) of final samples are assessed by SDS PAGE under reducing and non-reducing conditions, followed by immunoblotting using an anti-HSA or anti idiotype antibody as well as by analytical SEC, respectively. Purified proteins are stored at aliquots at −80° C. until use.

### Example 15: Determination of Antigen Affinity by Flow Cytometry

The activatable IFNγ proteins of Example 1 are tested for their binding affinities to human CD20⁺ cells and cynomolgus CD20 cells.

CD20 cells are incubated with 100 μL of serial dilutions of the activatable IFNγ proteins of Example 1 and at least one protease. After washing three times with FACS buffer the cells are incubated with 0.1 mL of 10 μg/mL mouse monoclonal anti-idiotype antibody in the same buffer for 45 min on ice. After a second washing cycle, the cells are incubated with 0.1 mL of 15 μg/mL FITC-conjugated goat anti-mouse IgG antibodies under the same conditions as before. As a control, cells are incubated with the anti-His IgG followed by the FITC-conjugated goat anti-mouse IgG antibodies without the activatable IFNγproteins. The cells were then washed again and resuspended in 0.2 mL of FACS buffer containing 2 μg/mL propidium iodide (PI) in order to exclude dead cells. The fluorescence of 1×10⁴ living cells is measured using a Beckman-Coulter FC500 MPL flow cytometer using the MXP software (Beckman-Coulter, Krefeld, Germany) or a Millipore Guava EasyCyte flow cytometer using the Incyte software (Merck Millipore, Schwalbach, Germany). Mean fluorescence intensities of the cell samples are calculated using CXP software (Beckman-Coulter, Krefeld, Germany) or Incyte software (Merck Millipore, Schwalbach, Germany). After subtracting the fluorescence intensity values of the cells stained with the secondary and tertiary reagents alone the values are then used for calculation of the K_{D} values with the equation for one-site binding (hyperbola) of the GraphPad Prism (version 6.00 for Windows, GraphPad Software, La Jolla California USA).

CD20 binding and crossreactivity are assessed on the human CD20⁺ tumor cell lines. The K_{D}ratio of crossreactivity is calculated using the K_{D} values determined on the CHO cell lines expressing either recombinant human or recombinant cynomolgus antigens.

### Example 16: Cytotoxicity Assay

The activatable IFNγ protein of Example 5 is evaluated in vitro on its mediation of immune response to CD20 target cells.

Fluorescence labeled CD20⁺ REC-1 cells (a Mantle cell lymphoma cell line, ATCC CRL-3004) are incubated with isolated PBMC of random donors or CB15 T-cells (standardized T-cell line) as effector cells in the presence of the activatable IFNγ protein of Example 5 and at least one protease. After incubation for 4 h at 37° C. in a humidified incubator, the release of the fluorescent dye from the target cells into the supernatant is determined in a spectrofluorimeter. Target cells incubated without the activatable IFNγ protein of Example 5 and target cells totally lysed by the addition of saponin at the end of the incubation serve as negative and positive controls, respectively.

Based on the measured remaining living target cells, the percentage of specific cell lysis is calculated according to the following formula: [1-(number of living targets₍ₛₐₘₚₗₑ₎/number of living targets₍ₛₚₒₙₜₐₙₑₒᵤₛ₎)]×100%. Sigmoidal dose response curves and EC₅₀ values are calculated by non-linear regression/4-parameter logistic fit using the GraphPad Software. The lysis values obtained for a given antibody concentration are used to calculate sigmoidal dose-response curves by 4 parameter logistic fit analysis using the Prism software.

### Example 17: Pharmacokinetics of activatable IFNγ Proteins

The activatable IFNγ protein of Example 5 is evaluated for half-time elimination in animal studies.

The activatable IFNγ protein is administered to cynomolgus monkeys as a 0.5 mg/kg bolus injection into the saphenous vein. Another cynomolgus monkey group receives a comparable cytokine in size, but lacking a serum half-life extension element. A third and fourth group receive a cytokine with serum half-life extension elements and a cytokine with CD20 and serum half-life extension elements respectively, and both comparable in size to the activatable IFNγ protein. Each test group consists of 5 monkeys. Serum samples are taken at indicated time points, serially diluted, and the concentration of the proteins is determined using a binding ELISA to CD20.

Pharmacokinetic analysis is performed using the test article plasma concentrations. Group mean plasma data for each test article conforms to a multi-exponential profile when plotted against the time post-dosing. The data are fit by a standard two-compartment model with bolus input and first-order rate constants for distribution and elimination phases. The general equation for the best fit of the data for i.v. administration is: c(t)=Ae^{-αt}+Be^{-βt}, where c(t) is the plasma concentration at time t, A and B are intercepts on the Y-axis, and α and βare the apparent first-order rate constants for the distribution and elimination phases, respectively. The α-phase is the initial phase of the clearance and reflects distribution of the protein into all extracellular fluid of the animal, whereas the second or β-phase portion of the decay curve represents true plasma clearance. Methods for fitting such equations are well known in the art. For example, A=D/V(α−k21)/(α−β), B=D/V(β−k21)/(α−β), and α and β(for α>β) are roots of the quadratic equation: r²⁺(k12⁺k21⁺k10)r+k2lk10=0 using estimated parameters of V=volume of distribution, k10=elimination rate, k12=transfer rate from compartment 1 to compartment 2 and k21=transfer rate from compartment 2 to compartment 1, and D=the administered dose.

Data analysis: Graphs of concentration versus time profiles are made using KaleidaGraph (KaleidaGraph™ V. 3.09 Copyright 1986-1997. Synergy Software. Reading, Pa.). Values reported as less than reportable (LTR) are not included in the PK analysis and are not represented graphically. Pharmacokinetic parameters are determined by compartmental analysis using WinNonlin software (WinNonlin® Professional V. 3.1 WinNonlin™ Copyright 1998-1999. Pharsight Corporation. Mountain View, Calif.). Pharmacokinetic parameters are computed as described in Ritschel W A and Kearns G L, 1999, IN: Handbook Of Basic Pharmacokinetics Including Clinical Applications, 5^{th} edition, American Pharmaceutical Assoc., Washington, D.C.

It is expected that the activatable IFNγ protein of Example 5 has improved pharmacokinetic parameters such as an increase in elimination half-time as compared to proteins lacking a serum half-life extension element.

### Example 18: Xenograft Tumor Model

The activatable IFNγ protein of Example 5 is evaluated in a xenograft model.

Female immune-deficient NOD/scid mice are sub-lethally irradiated (2 Gy) and subcutaneously inoculated with 4×10⁶ Ramos RA1 cells into the right dorsal flank. When tumors reach 100 to 200 mm³, animals are allocated into 3 treatment groups. Groups 2 and 3 (8 animals each) are intraperitoneally injected with 1.5×10⁷ activated human T-cells. Three days later, animals from Group 3 are subsequently treated with a total of 9 intravenous doses of 50 gg activatable IFNγ protein of Example 5 (qdx9d). Groups 1 and 2 are only treated with vehicle. Body weight and tumor volume are determined for 30 days.

It is expected that animals treated with the activatable IFNγ protein of Example 5 have a statistically significant delay in tumor growth in comparison to the respective vehicle-treated control group.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 19: HEK-Blue Assay

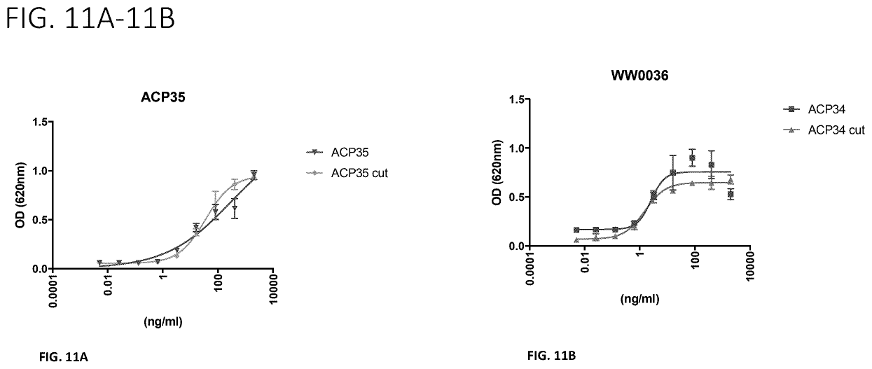

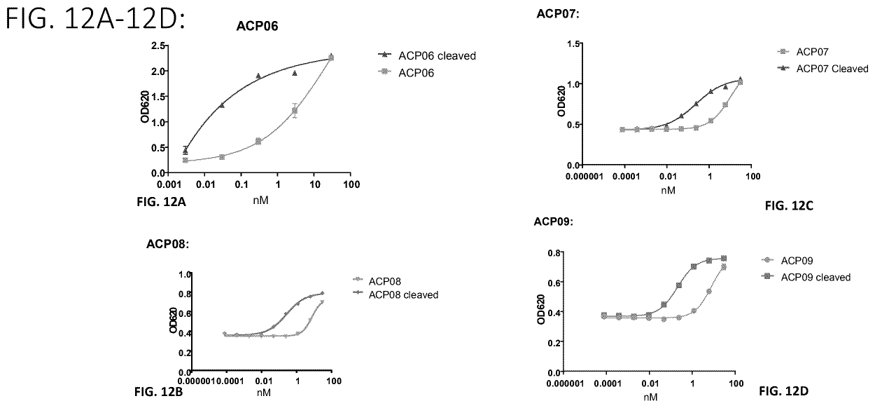

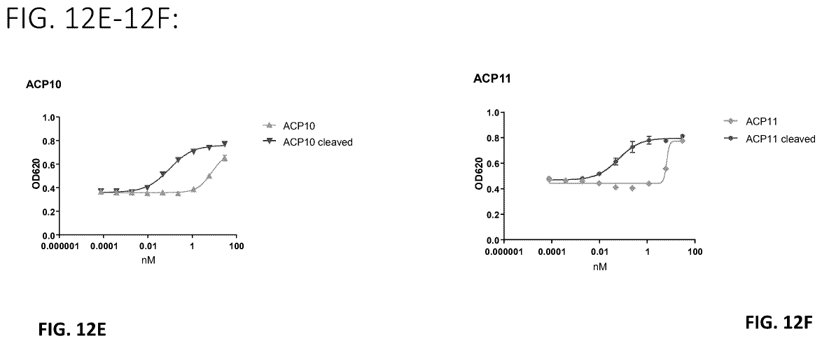

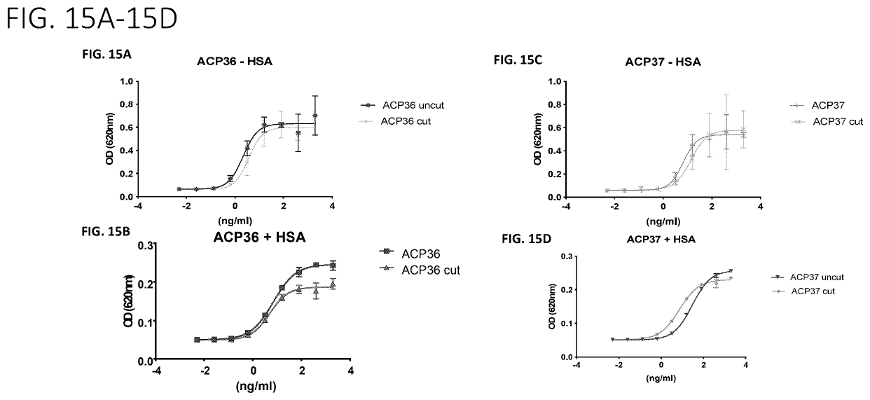

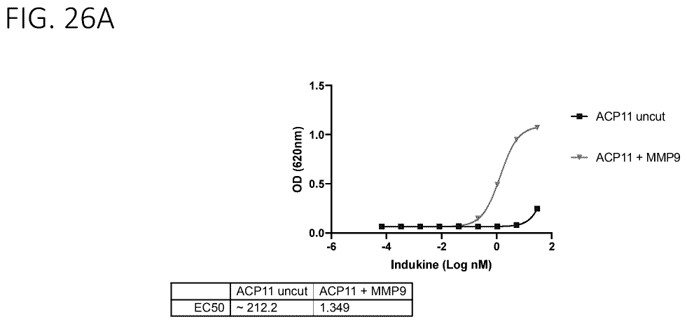

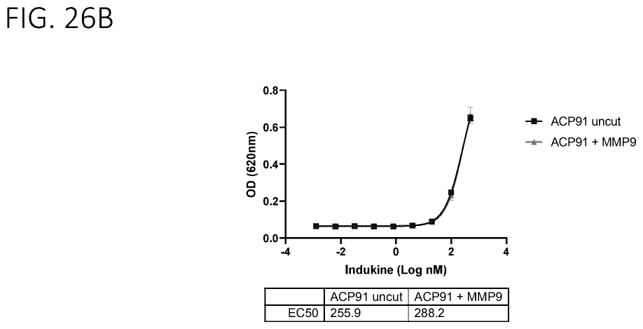

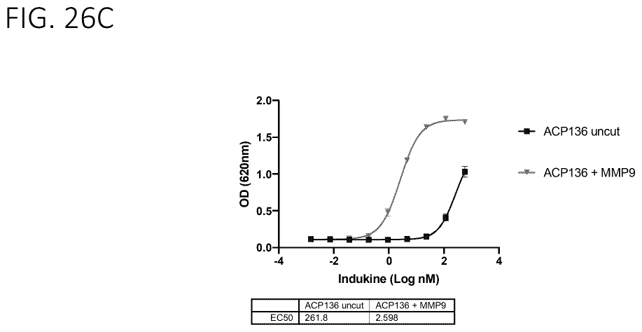

HEK-Blue IL12 cells (InvivoGen) were plated in suspension at a concentration of 250,000 cells/well in culture media with or without 40 mg/ml human serum albumin (HSA) and stimulated with a dilution series of recombinant hIL12, chimeric IL12 (mouse p35/human p40) or activatable hIL12 for 24 hours at 37° C. and 5% CO₂. Activity of uncleaved and cleaved activatable hIL12 was tested. Cleaved inducible hIL12 was generated by incubation with active MMP9. IL12 activity was assessed by quantification of Secreted Alkaline Phosphatase (SEAP) activity using the reagent QUANTI-Blue (InvivoGen), a colorimetric based assay. Results are shown in FIGS. 11, 12, 15, and 26.

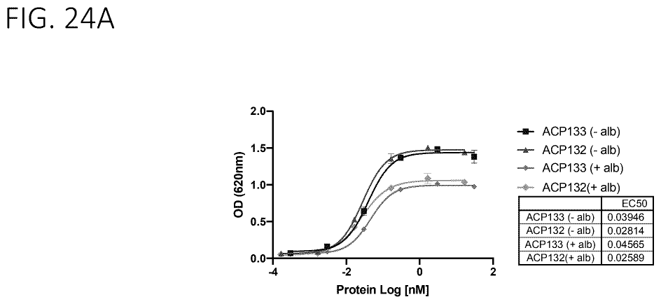

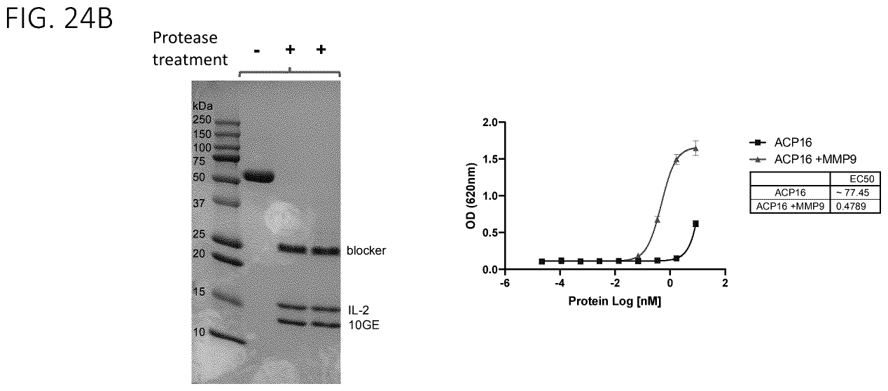

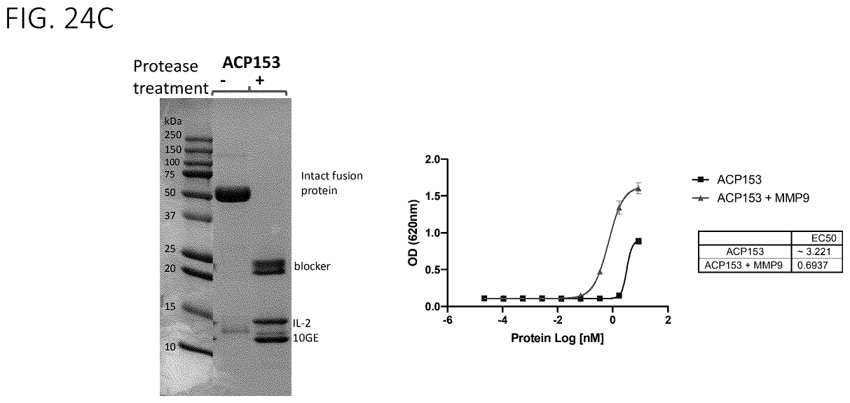

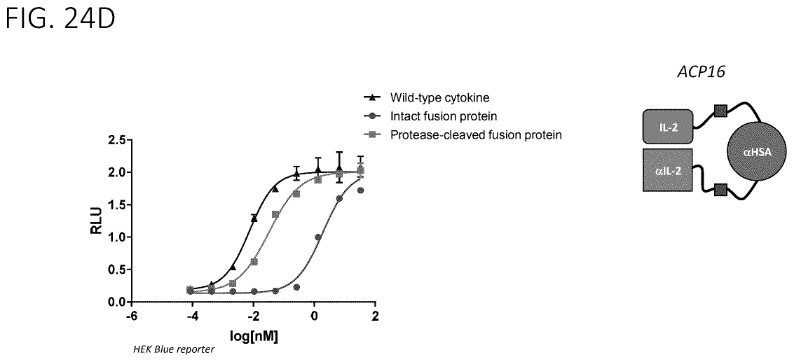

HEK-Blue IL2 cells (InvivoGen) were plated in suspension at a concentration of 50,000 cells/well in culture media with or without 15-40 mg/ml human serum albumin (HSA) and stimulated with a dilution series of recombinant hIL2 or activatable hIL2 for 24 hours at 3711C and 5% CO₂. Activity of uncleaved and cleaved activatable hIL2 was tested. Cleaved inducible hIL2 was generated by incubation with active MMP9. IL12 activity was assessed by quantification of Secreted Alkaline Phosphatase (SEAP) activity using the reagent QUANTI-Blue (InvivoGen), a colorimetric based assay. Results are shown in FIGS. 24A-24D.

### Example 20: Splenocyte T-Blast assay

T-Blasts were induced from murine splenocytes with a 6-day incubation with PHA and a 24 hr incubation with recombinant hIL12. Tblasts were then plated in suspension at a concentration of 200,000 cells/well in culture media with or without 40 mg/ml human serum albumin (HSA) and stimulated with a dilution series of recombinant hIL12 or chimeric IL12 (mouse p35/human p40) or mouse IL12 for 72 hours at 37° C. and 5% CO₂. Activity of uncleaved and cleaved IL12 fusion proteins was tested. Cleaved inducible hIL12 was generated by incubation with active MMP9. IL12 activity was assessed by downstream quantification of IFNγ production using a mIFNγ alpha ELISA.

### Example 21: In Vivo Delivery of a Protease Activated Fusion Protein Results in Decreased Tumor Growth

The chimeric polypeptide is examined to determine if it could have biological effects in vivo. For these experiments a system is used in which tumor cells injected intraperitoneally rapidly and preferentially attach and grow initially on the milky spots, a series of organized immune aggregates found on the omentum (Gerber et al., Am. J. Pathol. 169:1739-52 (2006)). This system offers a convenient way to examine the effects of fusion protein treatment on tumor growth since fusion proteins can be delivered intraperitoneally multiple times and tumor growth can be analyzed by examining the dissociated omental cells. For these experiments, the Colon 38 cell line, a rapidly growing tumor cell line that expresses both MMP2 and MMP9 in vitro, may be used. The omental tissue normally expresses a relatively small amount of MMP2 and MMP9, but, when Colon 38 tumor is present on the omentum, MMP levels increase. Using this tumor model, the ability of IL-2 mutein fusion proteins to affect tumor growth is examined. Colon 38 cells are injected intraperitoneally, allowed to attach and grow for 1 day, and then treated daily with fusion protein interaperitoneally. At day 7, the animals are sacrificed and the omenta examined for tumor growth using flow cytometry and by a colony-forming assay.

### Example 22: Construction of an Exemplary Activatable Interleukin Protein Targeting CD20

### Generation of an Activatable Interleukin Domain

The human IL-12p35 chain canonical sequence is Uniprot Accession No. P29459. The human IL-12p40 chain canonical sequence is Uniprot Accession No. P29460. IL-12p35 and IL-12p40 are cloned into an expression construct. A protease cleavage site is included between the IL-12p35 and IL-12p40 domains. An IL-12 polypeptide capable of binding to CD20 polypeptide present in a tumor or on a tumor cell is produced as follows. A nucleic acid is produced that contains nucleic acid sequences: (1) encoding an IFNγ polypeptide sequence and (2) one or more polypeptide linkers. Activatable interleukin plasmid constructs can have optional Flag, His or other affinity tags, and are electroporated into HEK293 or other suitable human or mammalian cell lines and purified. Validation assays include T cell activation assays using T cells responsive to IL-12 stimulation in the presence of a protease.

Generation of a scFv CD20 Binding Domain

CD20 is one of the cell surface proteins present on B-lymphocytes. CD20 antigen is found in normal and malignant pre-B and mature B lymphocytes, including those in over 90% of B-cell non-Hodgkin's lymphomas (NHL). The antigen is absent in hematopoietic stem cells, activated B lymphocytes (plasma cells) and normal tissue. As such, several antibodies mostly of murine origin have been described: 1F5, 2B8/C2B8, 2H7, and 1H4.

Human or humanized anti-CD20 antibodies are therefore used to generate scFv sequences for CD20 binding domains of an activatable interleukin protein. DNA sequences coding for human or humanized VL and VH domains are obtained, and the codons for the constructs are, optionally, optimized for expression in cells from Homo sapiens. The order in which the VL and VH domains appear in the scFv is varied (i.e., VL-VH, or VH-VL orientation), and three copies of the “G4S” (SEQ ID NO.: 449) or “G₄S” (SEQ ID NO.: 449) subunit (G₄S)₃ (SEQ ID NO.: 452) connect the variable domains to create the scFv domain. Anti-CD20 scFv plasmid constructs can have optional Flag, His or other affinity tags, and are electroporated into HEK293 or other suitable human or mammalian cell lines and purified. Validation assays include binding analysis by FACS, kinetic analysis using Proteon, and staining of CD20-expressing cells.

### Cloning of DNA Expression Constructs Encoding the Activatable Interleukin Protein

The activatable interleukin construct with protease cleavage site domains are used to construct an activatable interleukin protein in combination with an anti-CD20 scFv domain and a serum half-life extension element (e.g., a HSA binding peptide or VH domain). For expression of an activatable interleukin protein in CHO cells, coding sequences of all protein domains are cloned into a mammalian expression vector system. In brief, gene sequences encoding the activatable interleukin domain, serum half-life extension element, and CD20 binding domain along with peptide linkers L1 and L2 are separately synthesized and subcloned. The resulting constructs are then ligated together in the order of CD20 binding domain -L1-IL-12p35-L2-protease cleavage domain -L3-IL-12p40-L4-anti-CD20 scFv -L5-serum half-life extension element to yield a final construct. All expression constructs are designed to contain coding sequences for an N-terminal signal peptide and a C-terminal hexahistidine (6×His)-tag (SEQ ID NO.: 354) to facilitate protein secretion and purification, respectively.

### Expression of Activatable Interleukin Proteins in Stably Transfected CHO Cells

A CHO cell expression system (Flp-In®, Life Technologies), a derivative of CHO-K1 Chinese Hamster ovary cells (ATCC, CCL-61) (Kao and Puck, Proc. Natl. Acad Sci USA 1968; 60(4):1275-81), is used. Adherent cells are subcultured according to standard cell culture protocols provided by Life Technologies.

For adaption to growth in suspension, cells are detached from tissue culture flasks and placed in serum-free medium. Suspension-adapted cells are cryopreserved in medium with 10% DMSO.

Recombinant CHO cell lines stably expressing secreted activatable interleukin proteins are generated by transfection of suspension-adapted cells. During selection with the antibiotic Hygromycin B viable cell densities are measured twice a week, and cells are centrifuged and resuspended in fresh selection medium at a maximal density of 0.1×10⁶ viable cells/mL. Cell pools stably expressing activatable interleukin proteins are recovered after 2-3 weeks of selection at which point cells are transferred to standard culture medium in shake flasks. Expression of recombinant secreted proteins is confirmed by performing protein gel electrophoresis or flow cytometry. Stable cell pools are cryopreserved in DMSO containing medium.

Activatable interleukin proteins are produced in 10-day fed-batch cultures of stably transfected CHO cell lines by secretion into the cell culture supernatant. Cell culture supernatants are harvested after 10 days at culture viabilities of typically >75%. Samples are collected from the production cultures every other day and cell density and viability are assessed. On day of harvest, cell culture supernatants are cleared by centrifugation and vacuum filtration before further use.

Protein expression titers and product integrity in cell culture supernatants are analyzed by SDS-PAGE.

### Purification of Activatable Interleukin Proteins

Activatable interleukin proteins are purified from CHO cell culture supernatants in a two-step procedure. The constructs are subjected to affinity chromatography in a first step followed by preparative size exclusion chromatography (SEC) on Superdex 200 in a second step. Samples are buffer-exchanged and concentrated by ultrafiltration to a typical concentration of >1 mg/mL. Purity and homogeneity (typically >90%) of final samples are assessed by SDS PAGE under reducing and non-reducing conditions, followed by immunoblotting using an anti-HSA or anti idiotype antibody as well as by analytical SEC, respectively. Purified proteins are stored at aliquots at −80° C. until use.

### Example 23: Determination of Antigen Affinity by Flow Cytometry

The activatable interleukin proteins of Example 5 are tested for their binding affinities to human CD20⁺ cells and cynomolgus CD20 cells.

CD20 cells are incubated with 100 μL of serial dilutions of the activatable interleukin proteins of Example 5 and at least one protease. After washing three times with FACS buffer the cells are incubated with 0.1 mL of 10 μg/mL mouse monoclonal anti-idiotype antibody in the same buffer for 45 min on ice. After a second washing cycle, the cells are incubated with 0.1 mL of 15 μg/mL FITC-conjugated goat anti-mouse IgG antibodies under the same conditions as before. As a control, cells are incubated with the anti-His IgG followed by the FITC-conjugated goat anti-mouse IgG antibodies without the activatable interleukin proteins. The cells were then washed again and resuspended in 0.2 mL of FACS buffer containing 2 μg/mL propidium iodide (PI) in order to exclude dead cells. The fluorescence of 1×10⁴ living cells is measured using a Beckman-Coulter FC500 MPL flow cytometer using the MXP software (Beckman-Coulter, Krefeld, Germany) or a Millipore Guava EasyCyte flow cytometer using the Incyte software (Merck Millipore, Schwalbach, Germany). Mean fluorescence intensities of the cell samples are calculated using CXP software (Beckman-Coulter, Krefeld, Germany) or Incyte software (Merck Millipore, Schwalbach, Germany). After subtracting the fluorescence intensity values of the cells stained with the secondary and tertiary reagents alone the values are then used for calculation of the K_{D} values with the equation for one-site binding (hyperbola) of the GraphPad Prism (version 6.00 for Windows, GraphPad Software, La Jolla California USA).

CD20 binding and crossreactivity are assessed on the human CD20⁺ tumor cell lines. The K_{D}ratio of crossreactivity is calculated using the K_{D} values determined on the CHO cell lines expressing either recombinant human or recombinant cynomolgus antigens.

### Example 24: Cytotoxicity Assay

The activatable interleukin protein of Example 5 is evaluated in vitro on its mediation of immune response to CD20⁺ target cells.

Fluorescence labeled CD20 REC-1 cells (a Mantle cell lymphoma cell line, ATCC CRL-3004) are incubated with isolated PBMC of random donors or CB15 T-cells (standardized T-cell line) as effector cells in the presence of the activatable interleukin protein of Example 5 and at least one protease. After incubation for 4 h at 37° C. in a humidified incubator, the release of the fluorescent dye from the target cells into the supernatant is determined in a spectrofluorimeter. Target cells incubated without the activatable interleukin protein of Example 5and target cells totally lysed by the addition of saponin at the end of the incubation serve as negative and positive controls, respectively.

Based on the measured remaining living target cells, the percentage of specific cell lysis is calculated according to the following formula: [1-(number of living targets₍ₛₐₘₚₗₑ₎/number of living targets₍ₛₚₒₙₜₐₙₑₒᵤₛ₎)]×100%. Sigmoidal dose response curves and EC₅₀ values are calculated by non-linear regression/4-parameter logistic fit using the GraphPad Software. The lysis values obtained for a given antibody concentration are used to calculate sigmoidal dose-response curves by 4 parameter logistic fit analysis using the Prism software.

### Example 25: Pharmacokinetics of activatable interleukin Proteins

The activatable interleukin protein of Example 5 is evaluated for half-time elimination in animal studies.

The activatable interleukin protein is administered to cynomolgus monkeys as a 0.5 mg/kg bolus injection into the saphenous vein. Another cynomolgus monkey group receives a comparable cytokine in size, but lacking a serum half-life extension element. A third and fourth group receive a cytokine with serum half-life extension elements and a cytokine with CD20 and serum half-life extension elements respectively, and both comparable in size to the activatable interleukin protein. Each test group consists of 5 monkeys. Serum samples are taken at indicated time points, serially diluted, and the concentration of the proteins is determined using a binding ELISA to CD20.

Pharmacokinetic analysis is performed using the test article plasma concentrations. Group mean plasma data for each test article conforms to a multi-exponential profile when plotted against the time post-dosing. The data are fit by a standard two-compartment model with bolus input and first-order rate constants for distribution and elimination phases. The general equation for the best fit of the data for i.v. administration is: c(t)=Ae^{-αt}+Be^{-βt}, where c(t) is the plasma concentration at time t, A and B are intercepts on the Y-axis, and α and βare the apparent first-order rate constants for the distribution and elimination phases, respectively. The α-phase is the initial phase of the clearance and reflects distribution of the protein into all extracellular fluid of the animal, whereas the second or β-phase portion of the decay curve represents true plasma clearance. Methods for fitting such equations are well known in the art. For example, A=D/V(α−k21)/(α−β), B=D/V(β−k21)/(α−β), and α and β(for α>β) are roots of the quadratic equation: r²⁺(k12⁺k21⁺k10)r+k2lk10=0 using estimated parameters of V=volume of distribution, k10=elimination rate, k12=transfer rate from compartment 1 to compartment 2 and k21=transfer rate from compartment 2 to compartment 1, and D=the administered dose.

Data analysis: Graphs of concentration versus time profiles are made using KaleidaGraph (KaleidaGraph™ V. 3.09 Copyright 1986-1997. Synergy Software. Reading, Pa.). Values reported as less than reportable (LTR) are not included in the PK analysis and are not represented graphically. Pharmacokinetic parameters are determined by compartmental analysis using WinNonlin software (WinNonlin® Professional V. 3.1 WinNonlin™ Copyright 1998-1999. Pharsight Corporation. Mountain View, Calif.). Pharmacokinetic parameters are computed as described in Ritschel W A and Kearns G L, 1999, IN: Handbook Of Basic Pharmacokinetics Including Clinical Applications, 5^{th} edition, American Pharmaceutical Assoc., Washington, D.C.

It is expected that the activatable interleukin protein of Example 5 has improved pharmacokinetic parameters such as an increase in elimination half-time as compared to proteins lacking a serum half-life extension element.

### Example 26: Xenograft Tumor Model

The activatable interleukin protein of Example 5 is evaluated in a xenograft model.

Female immune-deficient NOD/scid mice are sub-lethally irradiated (2 Gy) and subcutaneously inoculated with 4×10⁶ Ramos RA1 cells into the right dorsal flank. When tumors reach 100 to 200 mm³, animals are allocated into 3 treatment groups. Groups 2 and 3 (8 animals each) are intraperitoneally injected with 1.5×10⁷ activated human T-cells. Three days later, animals from Group 3 are subsequently treated with a total of 9 intravenous doses of 50 gg activatable interleukin protein of Example 5 (qdx9d). Groups 1 and 2 are only treated with vehicle. Body weight and tumor volume are determined for 30 days.

It is expected that animals treated with the activatable interleukin protein of Example 5 have a statistically significant delay in tumor growth in comparison to the respective vehicle-treated control group.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 27: MC38 Experiments

The MC38 cell line, a rapidly growing colon adenocarcinoma cell line that expresses MMP9 in vitro, was used. Using this tumor model, the ability of fusion proteins to affect tumor growth was examined.

### Example 27a: MC38 IL-2POC

Agents and Treatment:

| | | | Formulation | | | |
|---|---|---|---|---|---|---|
| Gr. | N | Agent | dose | | Route | Schedule |
| 1^{#} | 10 | Vehicle | — | | ip | biwk x 3 |
| 2 | 7 | ACP16 | 700 | μg/animal | ip | biwk x 3 |
| 3 | 7 | ACP16 | 230 | μg/animal | ip | biwk x 3 |
| 4 | 7 | ACP16 | 70 | μg/animal | ip | biwk x 3 |
| 5 | 7 | ACP16 | 55 | ug/animal | ip | biwk x 3 |
| 6 | 7 | ACP16 | 17 | μg/animal | ip | biwk x 3 |
| 7 | 7 | ACP132 | 361 | μg/animal | ip | biwk x 3 |
| 8 | 7 | ACP132 | 119 | μg/animal | ip | biwk x 3 |
| 9 | 7 | ACP132 | 36 | μg/animal | ip | biwk x 3 |
| 10 | 7 | ACP132 | 28 | μg/animal | ip | biwk x 3 |
| 11 | 7 | ACP132 | 9 | μg/animal | ip | biwk x 3 |
| 12 | 7 | ACP21 | 540 | μg/animal | ip | biwk x 3 |
| 13 | 7 | ACP21 | 177 | μg/animal | ip | biwk x 3 |
| 14 | 7 | ACP21 | 54 | μg/animal | ip | biwk x 3 |
| 15 | 7 | ACP21 | 42 | μg/animal | ip | biwk x 3 |
| 16 | 7 | ACP21 | 13 | μg/animal | ip | biwk x 3 |
| # | −ControlGroup | | | | | |

### Procedures:

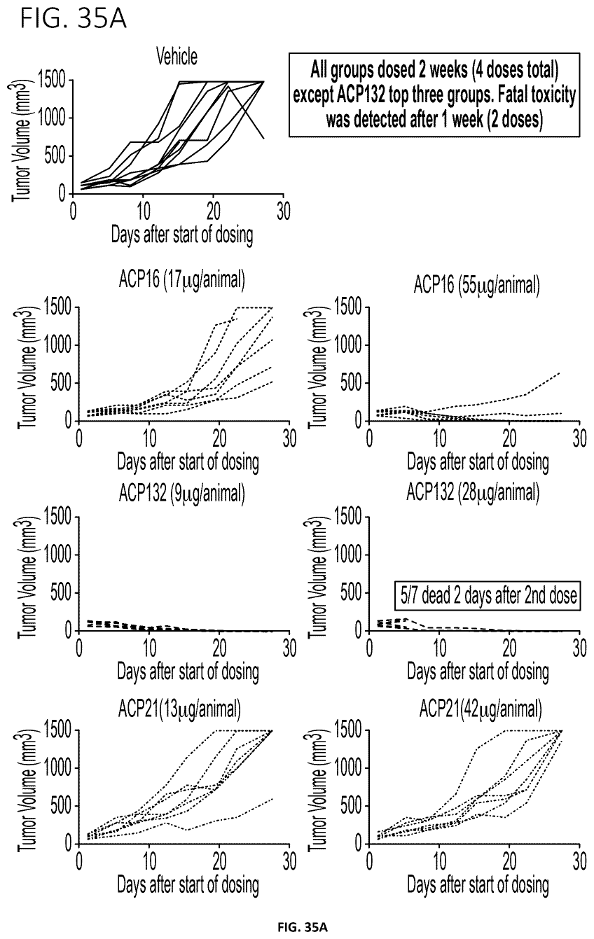

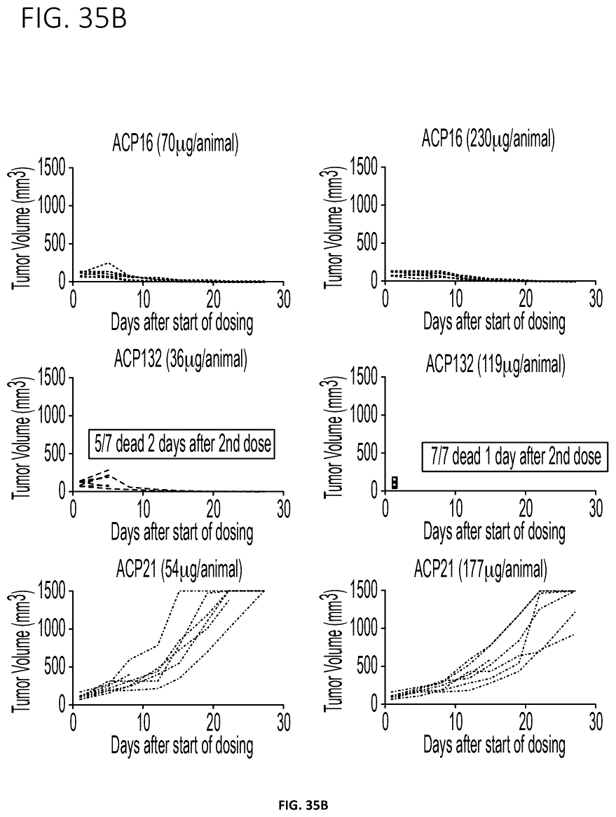

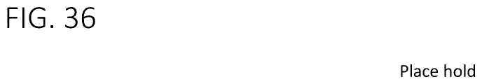

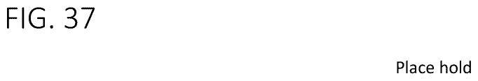

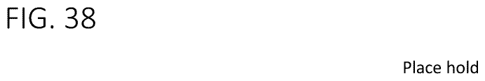

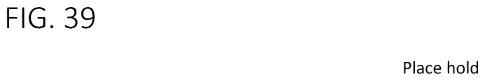

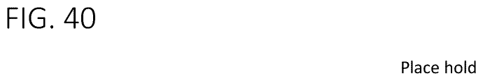

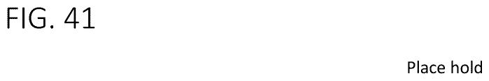

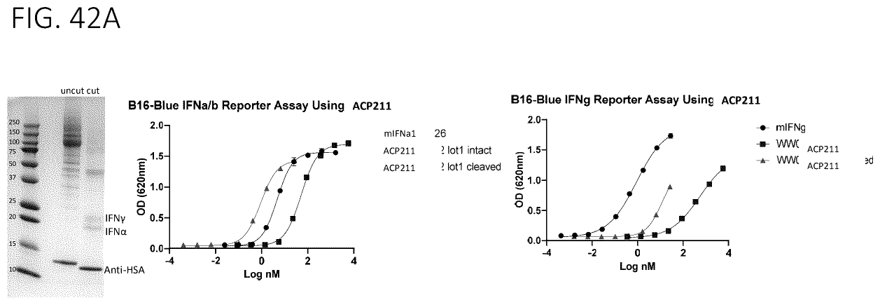

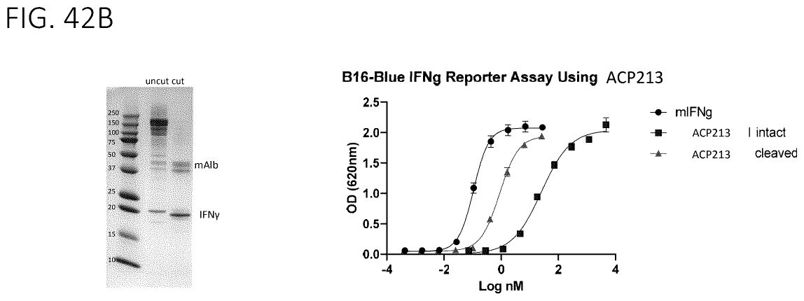

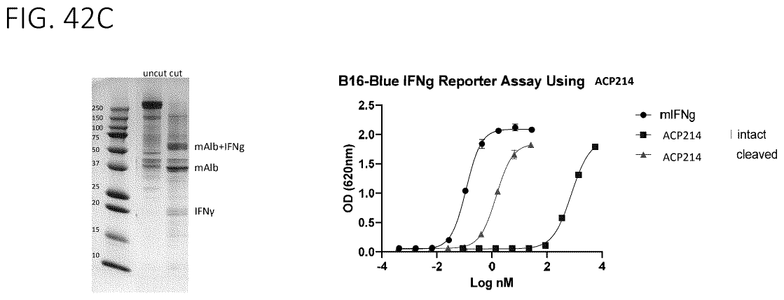

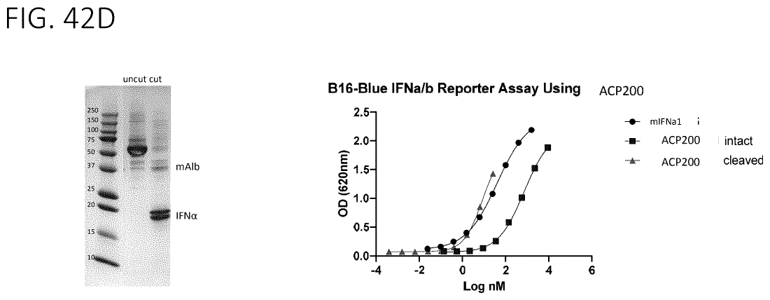

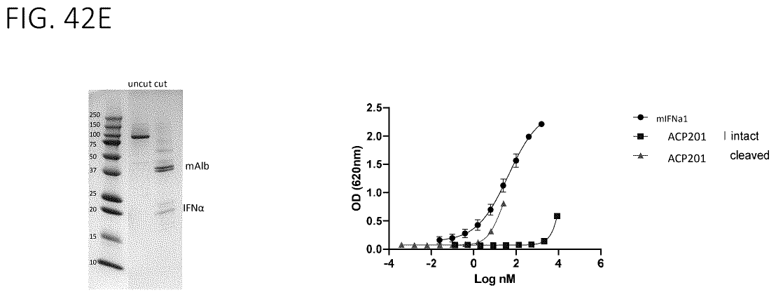

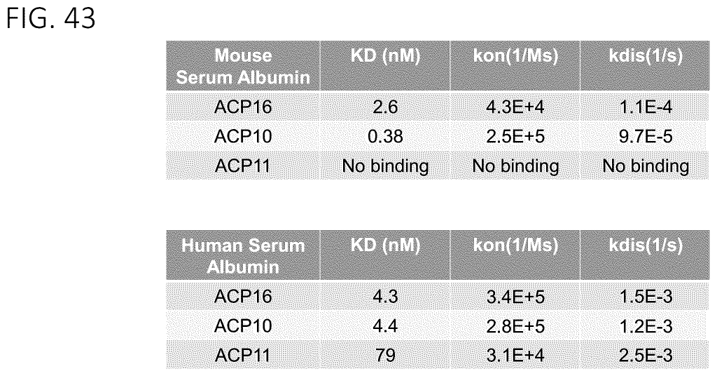

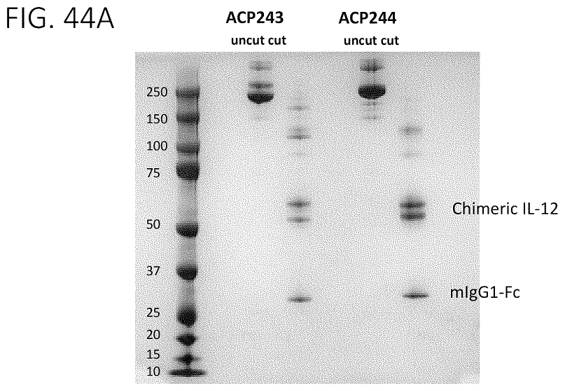

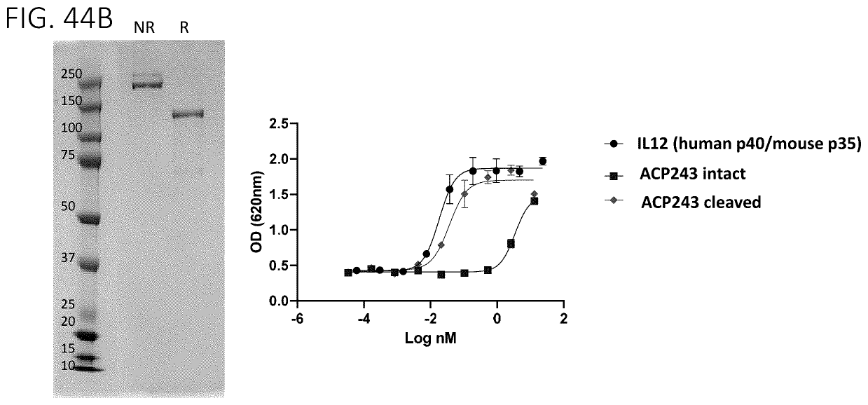

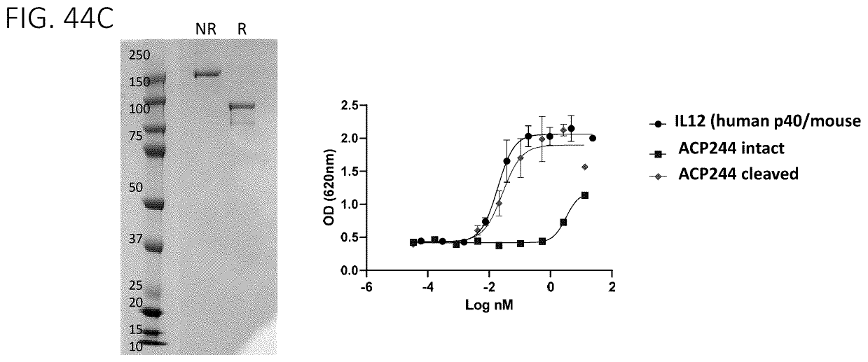

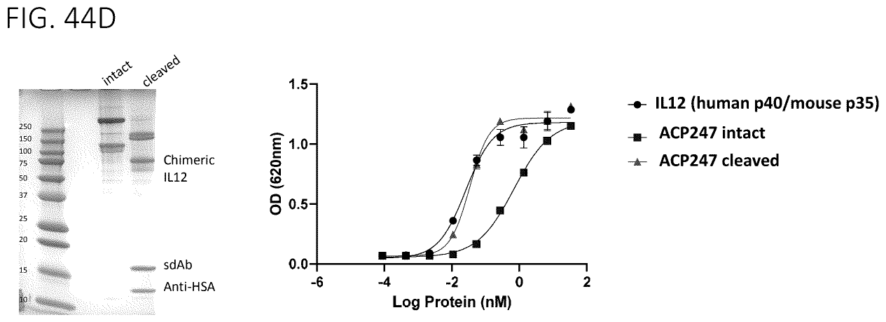

Mice were anaesthetized with isoflurane for implant of cells to reduce the ulcerations. 308 CR female C57BL/6 mice were set up with 5×10⁵ MC38 tumor cells in 0% Matrigel sc in flank. Cell Injection Volume was 0.1 mL/mouse. Mouse age at start date was 8 to 12 weeks. Pair matches were performed when tumors reach an average size of 100-150 mm³ and begin treatment. Body weights were taken at initiation and then biweekly to the end. Caliper measurements were taken biweekly to the end. Any adverse reactions were to be reported immediately. Any individual animal with a single observation of>than 30% body weight loss or three consecutive measurements of>25% body weight loss was euthanized. Any group with a mean body weight loss of>20% or >10% mortality stopped dosing; the group was not euthanized and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 45 days, whichever comes first. Responders were followed longer. When the endpoint was reached, the animals are to be euthanized. 13061 Results are shown in FIG. 35.

### Example 27b: MC38 IL-2 POC. Treatment with ACP16, ACP124 and ACP132

Agents and Treatment:

| | | | Formulation | | | |
|---|---|---|---|---|---|---|
| Gr. | N | Agent | dose | | Route | Schedule |
| 1^{#} | 12 | Vehicle | — | | ip | biwk x 2 |
| 2 | 8 | ACP16 | 4.4 | μg/animal | ip | biwk x 2 |
| 3 | 8 | ACP16 | 17 | μg/animal | ip | biwk x 2 |
| 4 | 8 | ACP16 | 70 | μg/animal | ip | biwk x 2 |
| 5 | 8 | ACP16 | 232 | μg/animal | ip | biwk x 2 |
| 6 | 8 | ACP130 | 19 | μg/animal | ip | biwk x 2 |
| 7 | 8 | ACP130 | 45 | μg/animal | ip | biwk x 2 |
| 8 | 8 | ACP130 | 180 | μg/animal | ip | biwk x 2 |
| 9 | 8 | ACP130 | 600 | μg/animal | ip | biwk x 1 |
| 12 | 8 | ACP124 | 17 | μg/animal | ip | biwk x 2 |
| 13 | 8 | ACP124 | 70 | μg/animal | ip | biwk x 2 |
| 14 | 8 | ACP124 | 230 | μg/animal | ip | biwk x 2 |
| 15 | 8 | ACP124 | 700 | μg/animal | ip | biwk x 2 |
| 16 | 8 | IL-2- | 12 | μg/animal | ip | bid x 5 then 2-day pause then |
| | | WTI | | | | bid x 5 then 2-day pause |
| 17 | 8 | IL-2- | 36 | μg/animal | ip | bid x 5 then 2-day pause then |
| | | WTI | | | | bid x 5 then 2-day pause |
| # | −Control | | | | | |
| | Group | | | | | |

### Procedures:

Mice were anaesthetized with isoflurane for implant of cells to reduce the ulcerations. 308 CR female C57BL/6 mice were set up with 5×10⁵ MC38 tumor cells in 0% Matrigel sc in flank. Cell Injection Volume was 0.1 mL/mouse. Mouse age at start date was 8 to 12 weeks. Pair matches were performed when tumors reach an average size of 100-150 mm³ and begin treatment. Body weights were taken at initiation and then biweekly to the end. Caliper measurements were taken biweekly to the end. Any adverse reactions were to be reported immediately. Any individual animal with a single observation of >than 30% body weight loss or three consecutive measurements of >25% body weight loss was euthanized. Any group with a mean body weight loss of >20% or >10% mortality stopped dosing; the group was not euthanized and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 45 days, whichever comes first. Responders were followed longer. When the endpoint was reached, the animals are to be euthanized.

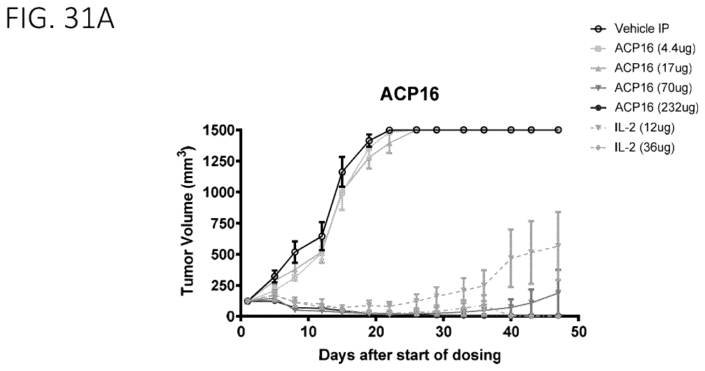

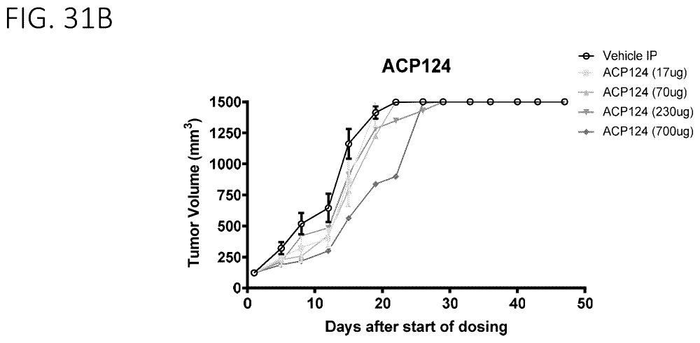

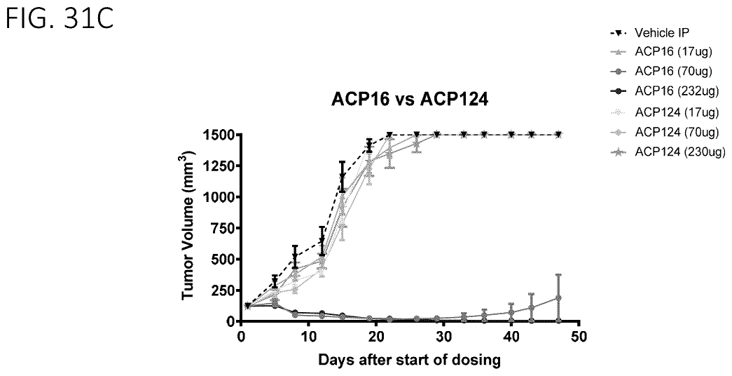

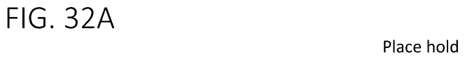

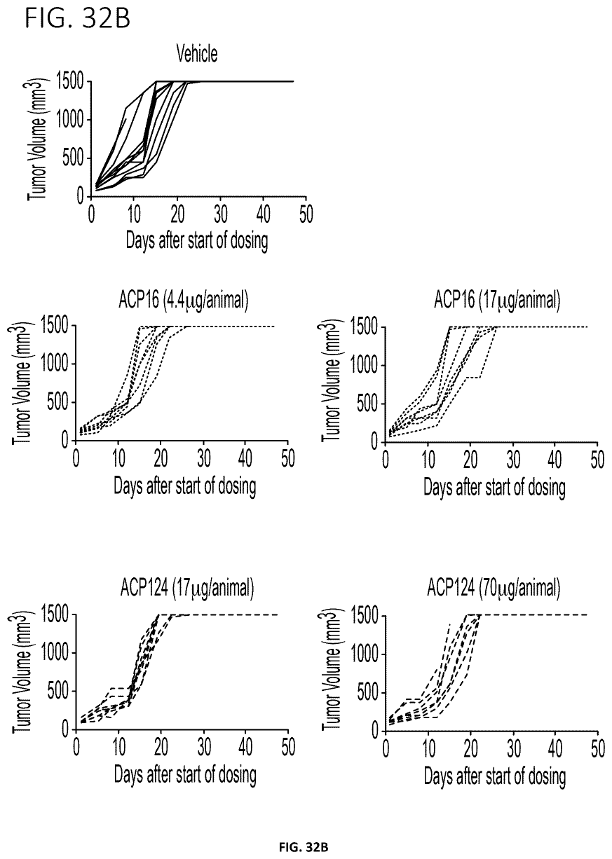

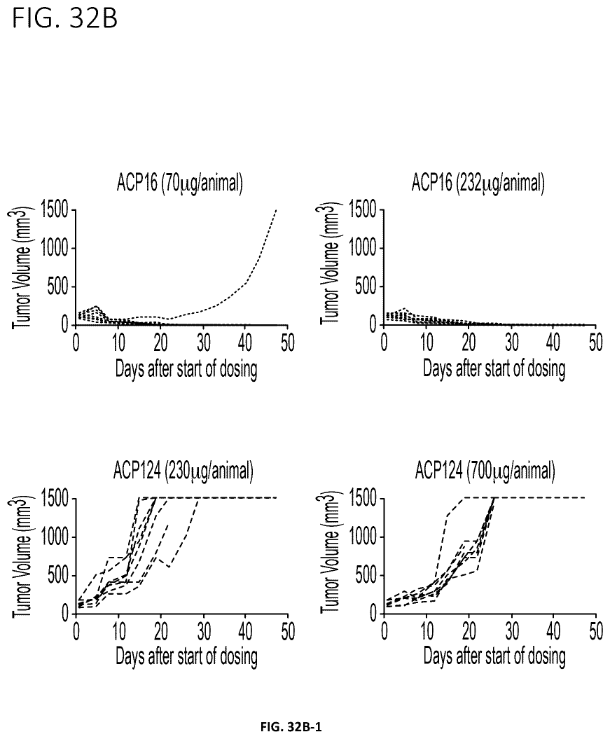

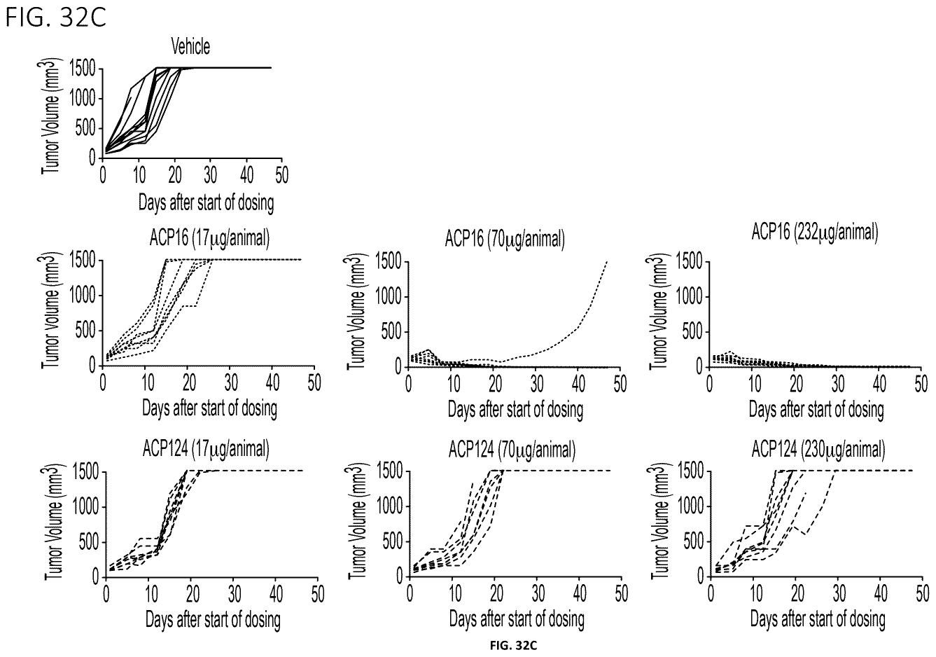

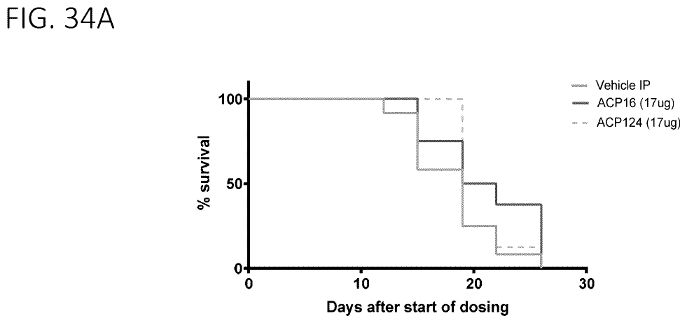

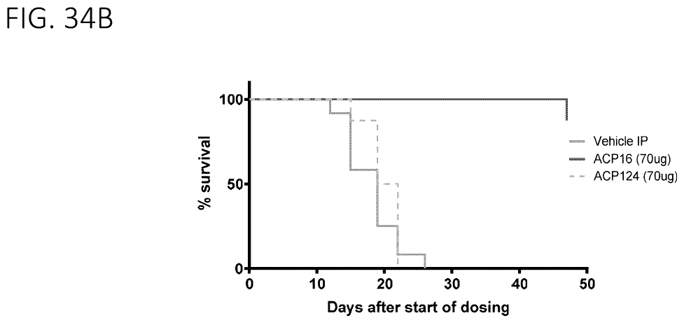

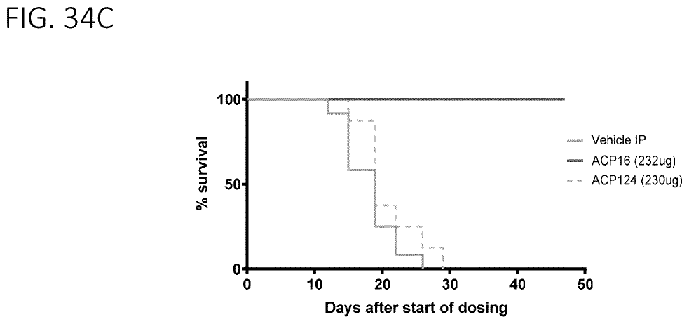

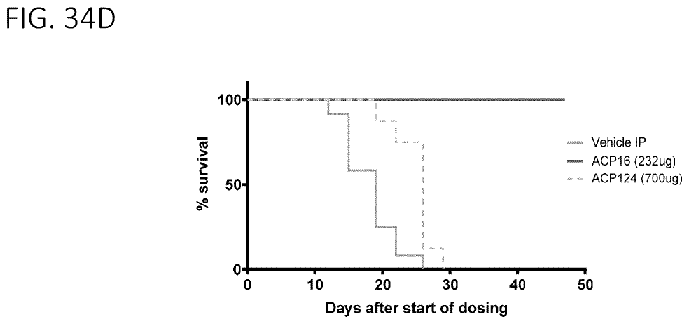

Results are shown in FIGS. 31A-31C and FIGS. 32B-32C. Survival curves are shown in FIGS. 34A-34D.

### Example 27c: MC38 IFNα and IL-12

Agents and Treatment:

| | | | Formulation | | | |
|---|---|---|---|---|---|---|
| Gr. | N | Agent | dose | | Route | Schedule |
| 1^{#} | 12 | Vehicle | — | | ip | biwk x 3 |
| 2 | 8 | ACP11 | 17.5 | μg/animal | ip | biwk x 3 |
| 3 | 8 | ACP11 | 175 | μg/animal | ip | biwk x 3 |
| 4 | 8 | ACP11 | 525 | μg/animal | ip | biwk x 3 |
| 5 | 8 | ACP31 | 33 | μg/animal | ip | biwk x 3 |
| 6 | 8 | ACP31 | 110 | μg/animal | ip | biwk x 3 |
| 7 | 8 | ACP31 | 330 | μg/animal | ip | biwk x 3 |
| 8 | 8 | ACP131 | 1 | μg/animal | ip | bid x 5 then 2-day pause then bid x 5 then 2- |
| | | | | | | day pause |
| 9 | 8 | ACP131 | 10 | μg/animal | ip | bid x 5 then 2-day pause then bid x 5 then 2- |
| | | | | | | day pause |
| 10 | 8 | ACP131 | 30 | μg/animal | ip | bid x 5 then 2-day pause then bid x 5 then 2- |
| | | | | | | day pause |
| 11 | 8 | mIFNa1-WTI | 1 | μg/animal | ip | bid x 5 then 2-day pause then bid x 5 then 2- |
| | | | | | | day pause |
| 12 | 8 | mIFNa1-WTI | 10 | μg/animal | ip | bid x 5 then 2-day pause then bid x 5 then 2- |
| | | | | | | day pause |
| 13 | 8 | IL-12-HM-WTI | 2 | μg/animal | ip | bid x 5 then 2-day pause then bid x 5 then 2- |
| | | | | | | day pause |
| 14 | 8 | IL-12-HM-WTI | 10 | μg/animal | ip | bid x 5 then 2-day pause then bid x 5 then 2- |
| | | | | | | day pause |
| 15 | 8 | ACP131 | 5 | μg/animal | itu | bid x 5 then 2-day pause then bid x 5 then 2- |
| | | | | | | day pause |
| # | −Control | | | | | |
| | Group | | | | | |

### Procedures:

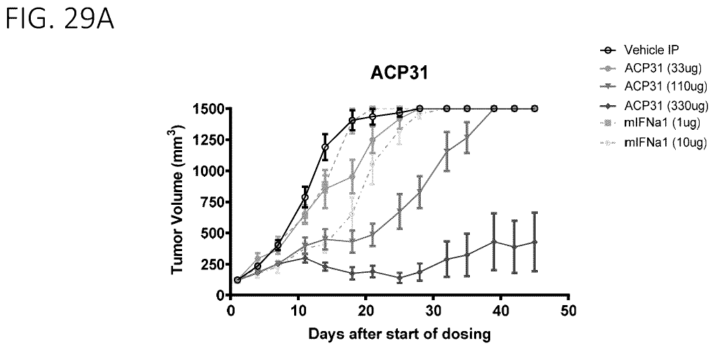

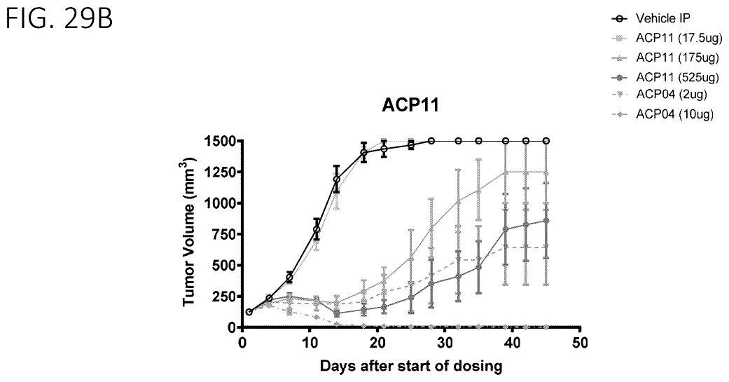

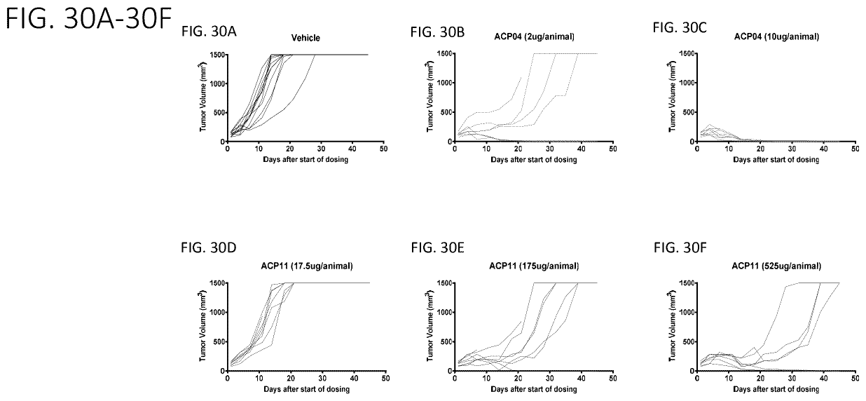

Mice were anaesthetized with isoflurane for implant of cells to reduce the ulcerations. 308 CR female C57BL/6 mice were set up with 5×10⁵ MC38 tumor cells in 0% Matrigel sc in flank. Cell Injection Volume was 0.1 mL/mouse. Mouse age at start date was 8 to 12 weeks. Pair matches were performed when tumors reach an average size of 100-150 mm³ and begin treatment. Body weights were taken at initiation and then biweekly to the end. Caliper measurements were taken biweekly to the end. Any adverse reactions were to be reported immediately. Any individual animal with a single observation of >than 30% body weight loss or three consecutive measurements of >25% body weight loss was euthanized. Any group with a mean body weight loss of >20% or >10% mortality stopped dosing; the group was not euthanized and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 45 days, whichever comes first. Responders were followed longer. When the endpoint was reached, the animals are to be euthanized. Results are show in in FIGS. 29A-29B, and 30A-30F.

### Example 27d: Treatment with ACP16, ACP132, and ACP21

Agents and Treatment:

| | | | Formulation | | | |
|---|---|---|---|---|---|---|
| Gr. | N | Agent | dose | | Route | Schedule |
| 1^{#} | 10 | Vehicle | — | | ip | biwk x 2 |
| 2 | 7 | ACP16 | 17 | μg/animal | ip | biwk x 2 |
| 3 | 7 | ACP16 | 55 | μg/animal | ip | biwk x 2 |
| 4 | 7 | ACP16 | 70 | μg/animal | ip | biwk x 2 |
| 5 | 7 | ACP16 | 230 | μg/animal | ip | biwk x 2 |
| 6 | 7 | ACP132 | 9 | μg/animal | ip | biwk x 2 |
| 7 | 7 | ACP132 | 28 | μg/animal | ip | biwk x 1 |
| 8 | 7 | ACP132 | 36 | μg/animal | ip | biwk x 1 |
| 9 | 7 | ACP132 | 119 | μg/animal | ip | biwk x 1 |
| 10 | 7 | ACP21 | 13 | μg/animal | ip | biwk x 2 |
| 11 | 7 | ACP21 | 42 | μg/animal | ip | biwk x 2 |
| 12 | 7 | ACP21 | 54 | μg/animal | ip | biwk x 2 |
| 13 | 7 | ACP21 | 177 | μg/animal | ip | biwk x 2 |

### Procedures:

Mice were anaesthetized with isoflurane for implant of cells to reduce the ulcerations. CR female C57BL/6 mice were set up with 5×10⁵ MC38 tumor cells in 0% Matrigel sc in flank. Cell Injection Volume was 0.1 mL/mouse. Mouse age at start date was 8 to 12 weeks. Pair matches were performed when tumors reach an average size of 100-150 mm³ and begin treatment. ACP16 was dosed at 17, 55, 70, or 230 μg/animal; ACP132 was dosed at 9, 28, 36, or 119ug/animal; ACP21 was dosed at 13, 42, 54, or 177 μg/animal. Body weights were taken at initiation and then biweekly to the end. Caliper measurements were taken biweekly to the end. Any adverse reactions were to be reported immediately. Any individual animal with a single observation of >than 30% body weight loss or three consecutive measurements of >25% body weight loss was euthanized. Any group with a mean body weight loss of >20% or >1 0% mortality stopped dosing; the group was not euthanized and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 45 days, whichever comes first. Responders were followed longer. When the endpoint was reached, the animals are to be euthanized. Results are shown in FIG. 35.

### Example 27e: MC38 Rechallenge

Cured mice (ACP16-treated) from Example 27b were rechallenged with tumor implantation to determine whether anti-tumor memory had been established from the initial treatments.

Agents and Treatment:

| | | | Formulation | | | |
|---|---|---|---|---|---|---|
| Gr. | N | Agent | dose | | Route | Schedule |
| 1^{#} | 33 | No | — | | — | — |
| | | Treatment | | | | |
| 2 | 7 | ACP16 | 70 | μg/animal | ip | (ACP16 biwkx2) |
| 3 | 8 | ACP16 | 232 | μg/animal | ip | (ACP16 biwkx2) |
| 5 | 5 | IL-2-WTI | 12 | μg/animal | ip | (IL-2-WTI bid x 5 then |
| | | | | | | 2-day pause then bid x |
| | | | | | | 5 then 2-day pause) |
| 6 | 7 | IL-2-WTI | 36 | μg/animal | ip | (IL-2-WTI bid x 5 then |
| | | | | | | 2-day pause then bid x |
| | | | | | | 5 then 2-day pause) |
| # | −Control | | | | | |
| | Group | | | | | |

### Procedures:

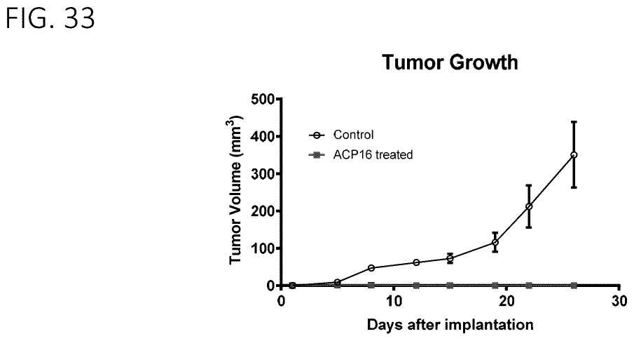

Mice were anaesthetized with isoflurane for implant of cells to reduce the ulcerations. This portion of the study began on the day of implant (Day 1). Group 1 consisted of 33 CR female C57BL/6 mice set up with 5×10⁵ MC38 tumor cells in 0% Matrigel subcutaneously in the flank. Groups 2-6 consisted of 33 CR female C57BL/6 mice set up with 5×10⁵ MC38 tumor cells in 0% Matrigel sc in the left flank. The tumors from the previous MC38 experiment (Example 27b) were implanted in the right flank of each animal. Cell Injection Volume was 0.1 mL/mouse. Age of control mice at initiation was 14 to 17 weeks. These mice were age matched to mice from the previous MC38 experiment (Example 27b). No dosing of active agent occurred during rechallenge. Body Weights were take biweekly until end, as were caliper measurements. Any adverse reactions or death were reported immediately. Any individual animal with a single observation of >than 30% body weight loss or three consecutive measurements of >25% body weight loss was euthanized. Endpoint was tumor growth delay (TGD). Animals were monitored individually. The endpoint of the experiment was a tumor volume of 1000 mm³ or 45 days, whichever comes first. Responders were followed longer when possible. When the endpoint is reached, the animals were euthanized. Results are shown in FIG. 33.

### Example 27f: Treatment with ACP10, ACP11

Agents and Treatment:

| | | | Formulation | | | |
|---|---|---|---|---|---|---|
| Gr. | N | Agent | dose | | Route | Schedule |
| 1^{#} | 12 | Vehicle | — | | ip | biwk x 2 |
| 2 | 8 | ACP11 | 175 | μg/animal | ip | biwk x 2 |
| 3 | 8 | ACP11 | 300 | μg/animal | ip | biwk x 2 |
| 4 | 8 | ACP10 | 5 | μg/animal | ip | biwk x 2 |
| 5 | 8 | ACP10 | 10 | μg/animal | ip | biwk x 2 |
| 6 | 8 | ACP10 | 43 | μg/animal | ip | biwk x 2 |
| 7 | 8 | ACP10 | 43 | μg/animal | ip | qwk x 2 |
| 8 | 8 | ACP10 | 172 | μg/animal | ip | biwk x 2 |
| 9 | 8 | IL-I2- | 5 | μg/animal | ip | bid for 5 days first day 1 dose then |
| | | HM-WTI | | | | 2-day pause then bid for 5 days first |
| | | | | | | day 1 dose then 2-day pause |
| 10 | 8 | IL-12- | 20 | μg/animal | ip | bid for 5 days first day 1 dose then |
| | | HM-WTI | | | | 2-day pause then bid for 5 days first |
| | | | | | | day 1 dose then 2-day pause |

### Procedures:

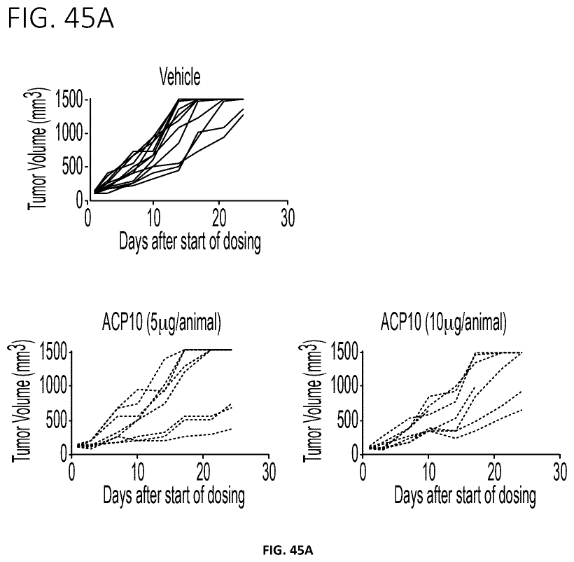

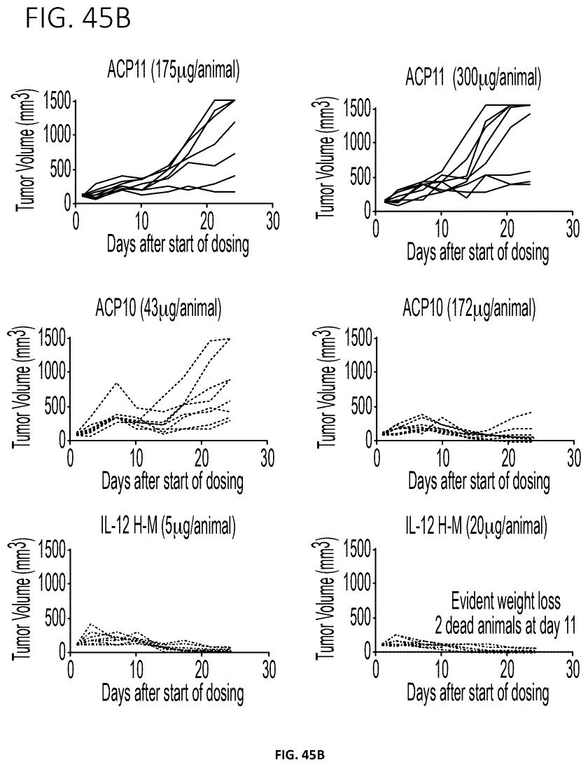

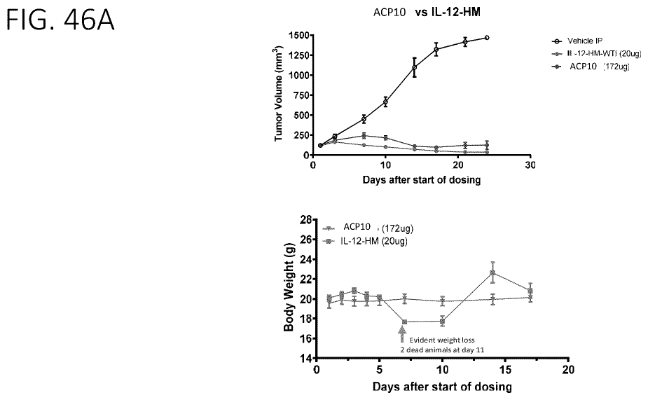

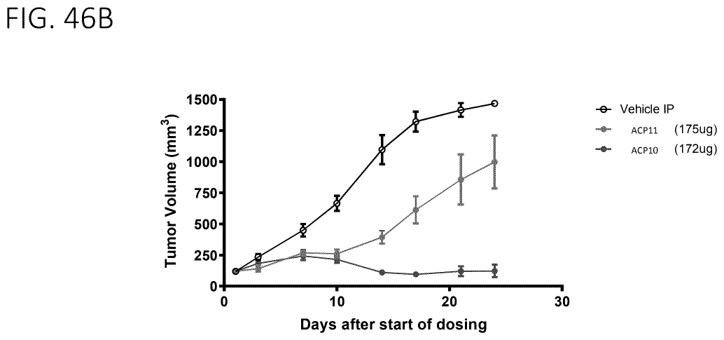

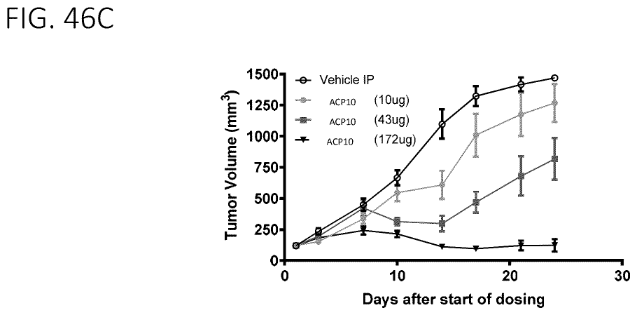

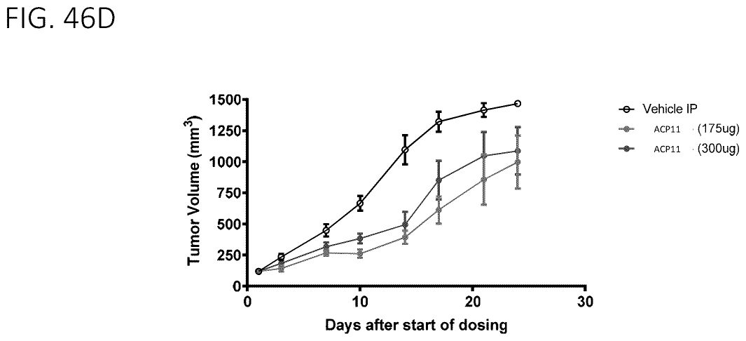

Mice were anaesthetized with isoflurane for implant of cells to reduce the ulcerations. CR female C57BL/6 mice were set up with 5×10⁵ MC38 tumor cells in 0% Matrigel sc in flank. Cell Injection Volume was 0.1 mL/mouse. Mouse age at start date was 8 to 12 weeks. Pair matches were performed when tumors reach an average size of 100-150 mm³ and begin treatment. ACP1I1 was dosed at 175 or 300 jig/animal; ACP10 was dosed at 5, 10, 43, or 172 ug/animal; IL-12-HM-WTI was dosed at 5 or 20 ug/animal. Body weights were taken at initiation and then biweekly to the end. Caliper measurements were taken biweekly to the end. Any adverse reactions were to be reported immediately. Any individual animal with a single observation of >than 30% body weight loss or three consecutive measurements of >25% body weight loss was euthanized. Any group with a mean body weight loss of >20% or >1 0% mortality stopped dosing; the group was not euthanized and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 45 days, whichever comes first. Responders were followed longer. When the endpoint was reached, the animals are to be euthanized. Results are shown in FIG. 45 and FIGS. 46A-46D.

### Example 27g: Treatment with ACP16, APC153, ACP155, ACP156 and ACP292

Agents and Treatment:

| | | | Formulation | | | |
|---|---|---|---|---|---|---|
| Gr. | N | Agent | dose | | Route | Schedule |
| 1^{#} | 12 | Vehicle | — | | ip | biwk x 2 |
| 2 | 8 | ACP16 | 17 | μg/animal | ip | biwk x 2 |
| 3 | 8 | ACP16 | 55 | μg/animal | ip | biwk x 2 |
| 4 | 8 | ACP16 | 230 | μg/animal | ip | biwk x 2 |
| 5 | 8 | ACP155 | 55 | μg/animal | ip | biwk x 2 |
| 6 | 8 | ACP155 | 230 | μg/animal | ip | biwk x 2 |
| 7 | 8 | ACP153 | 55 | μg/animal | ip | biwk x 2 |
| 8 | 8 | ACP153 | 230 | μg/animal | ip | biwk x 2 |
| 9 | 8 | ACP156 | 55 | μg/animal | ip | biwk x 2 |
| 10 | 8 | ACP156 | 230 | μg/animal | ip | biwk x 2 |
| 11 | 8 | ACP292 | 45 | μg/animal | ip | biwk x 2 |
| 12 | 8 | ACP292 | 186 | μg/animal | ip | biwk x 2 |

### Procedures:

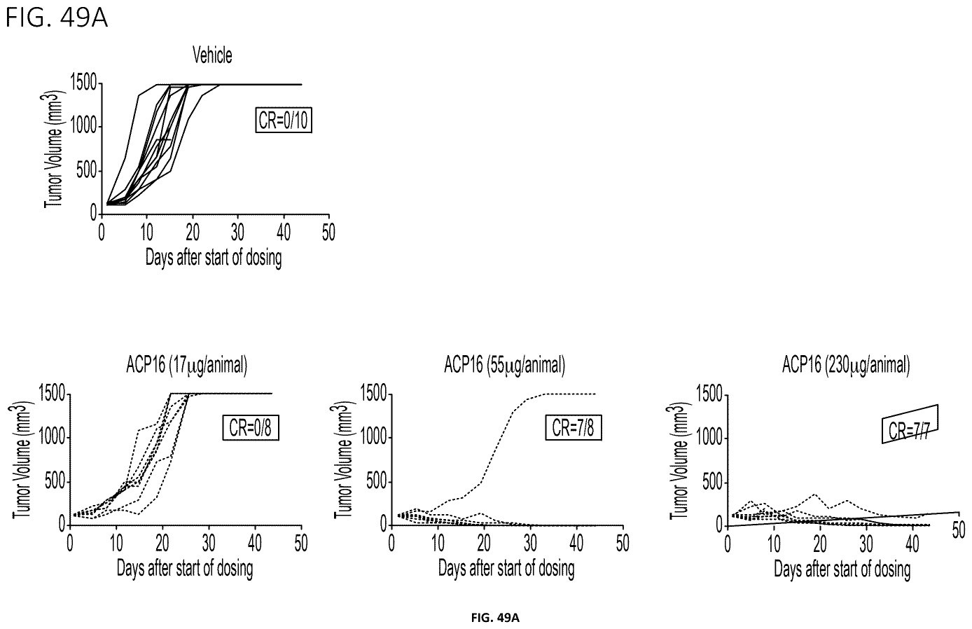

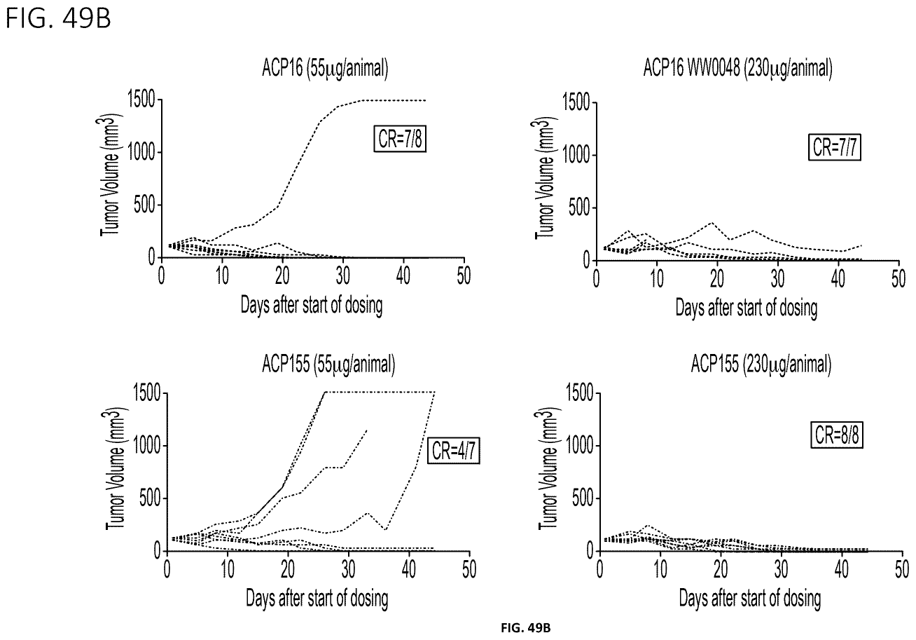

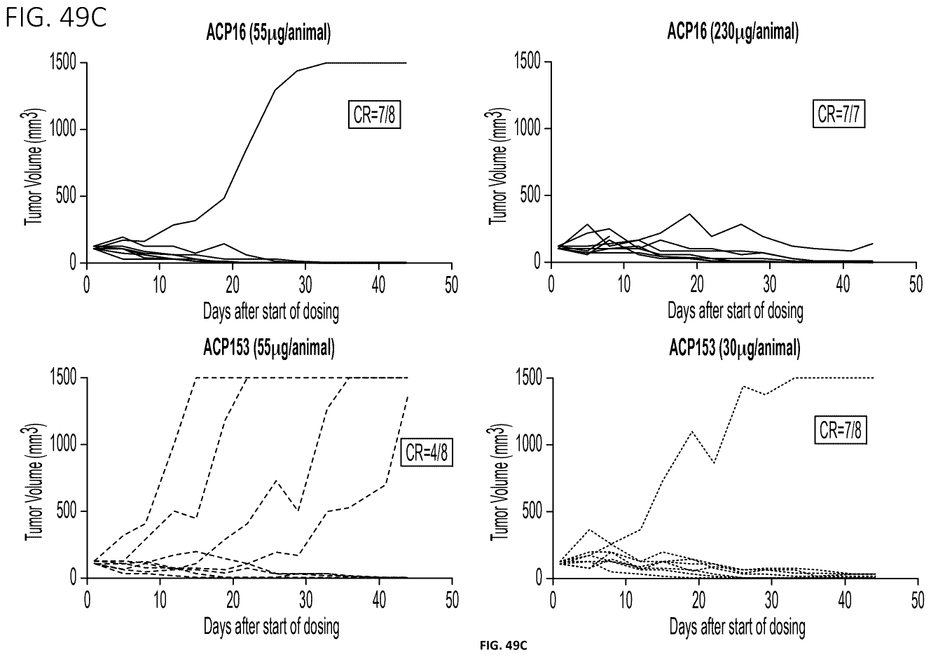

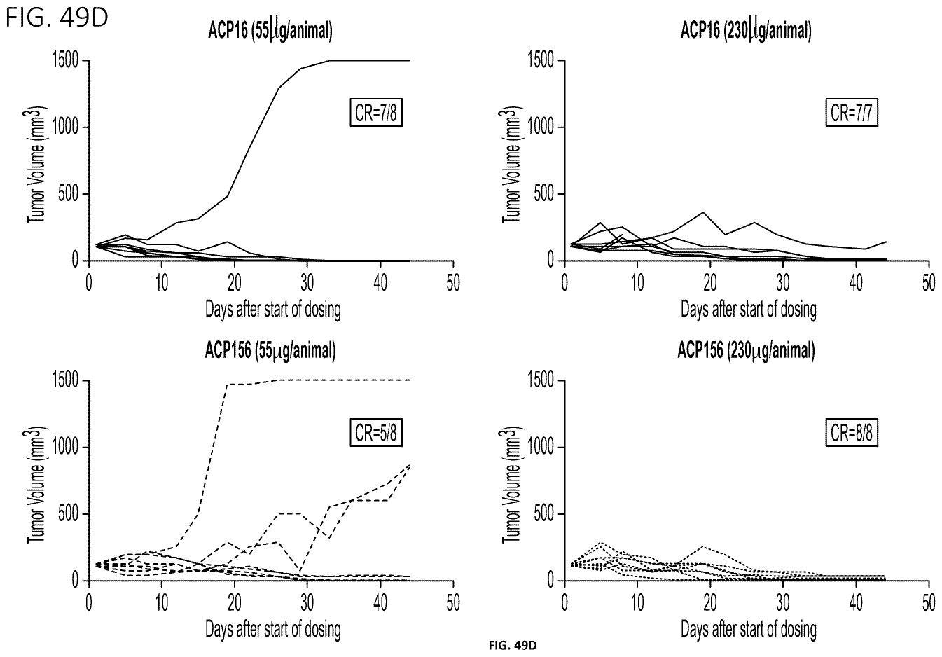

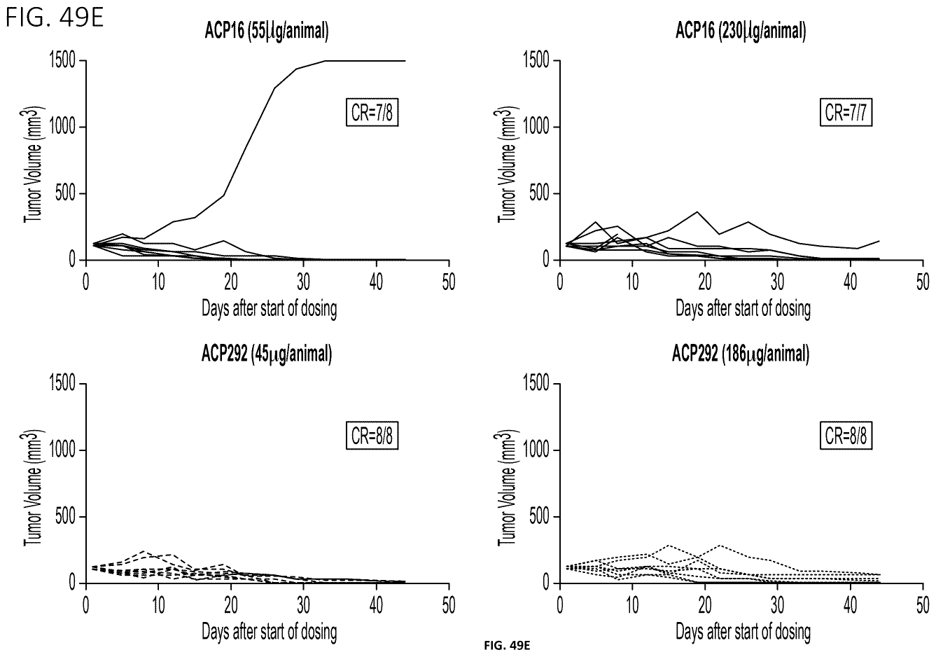

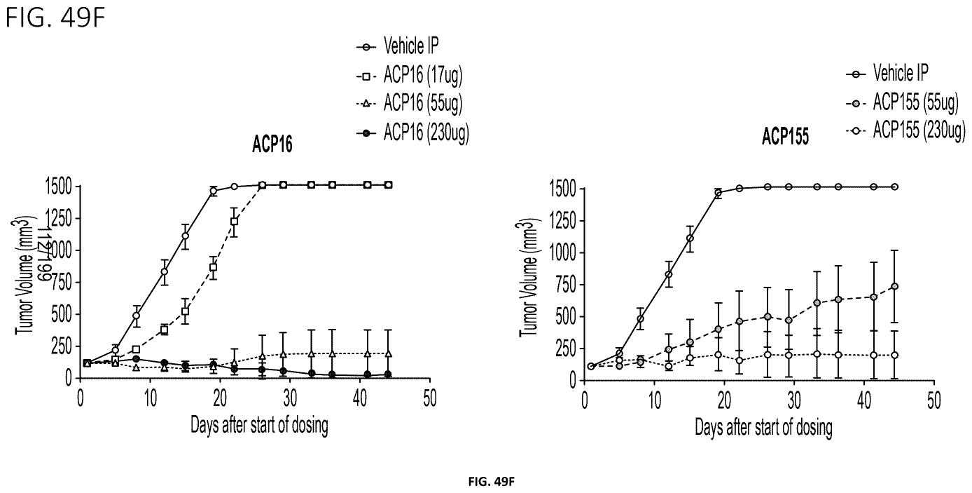

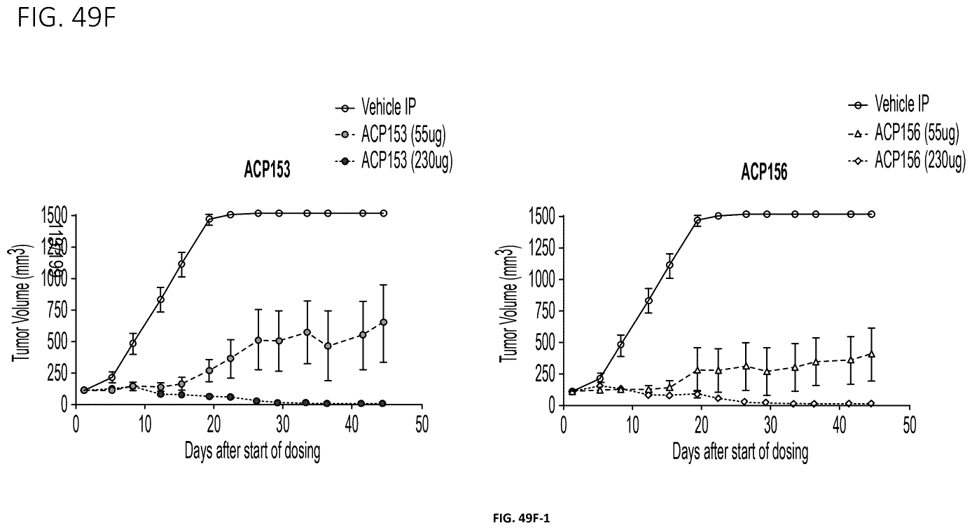

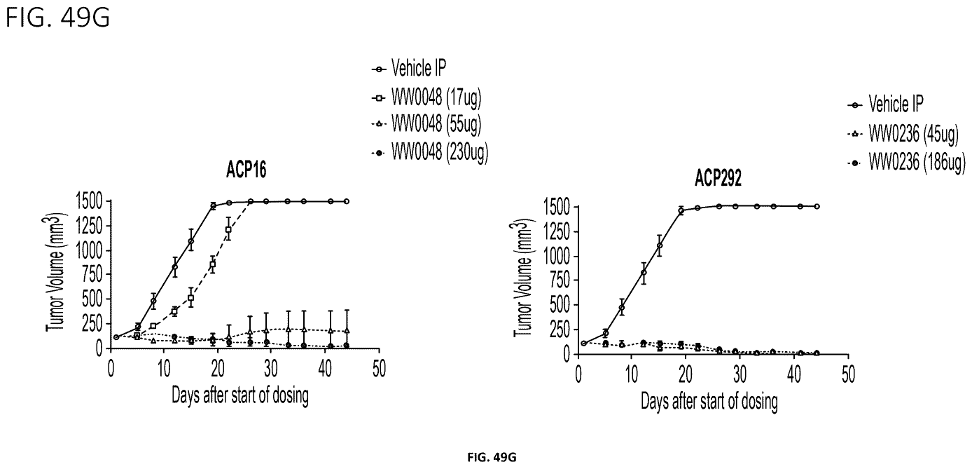

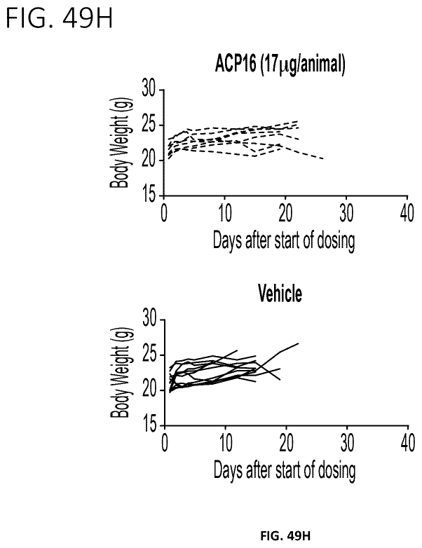

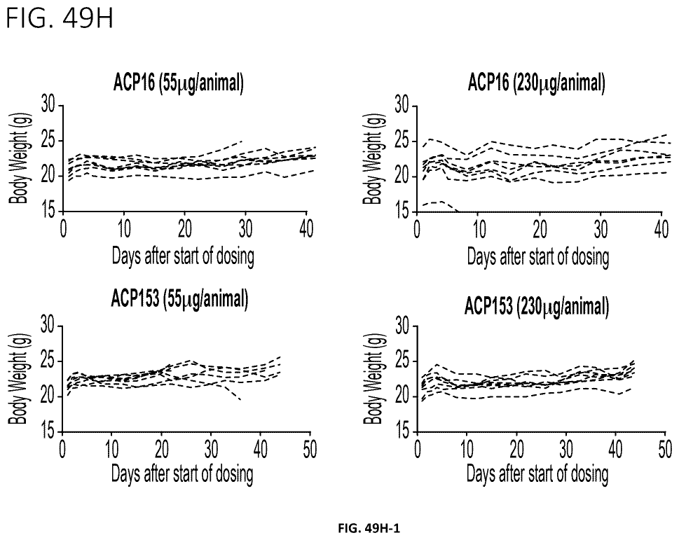

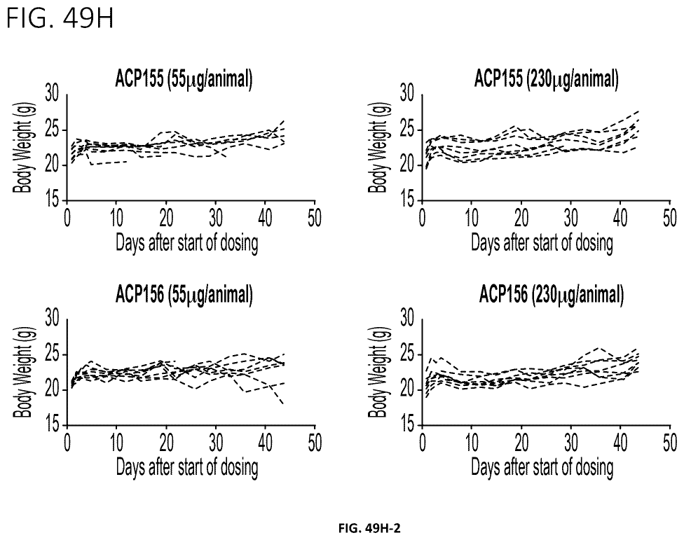

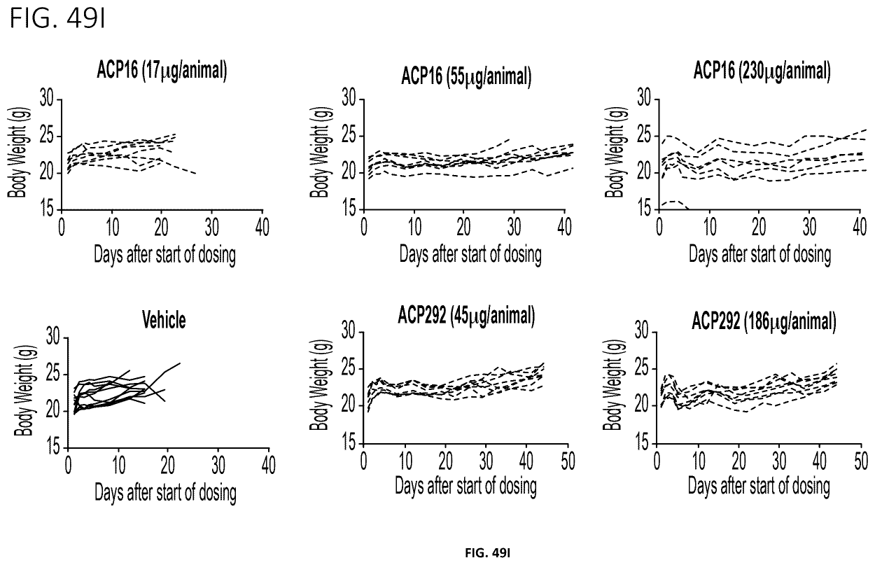

Mice were anaesthetized with isoflurane for implant of cells to reduce the ulcerations. CR female C57BL/6 mice were set up with 5×10⁵ MC38 tumor cells in 0% Matrigel sc in flank. Cell Injection Volume was 0.1 mL/mouse. Mouse age at start date was 8 to 12 weeks. Pair matches were performed when tumors reach an average size of 100-150 mm³ and begin treatment. ACP16 was dosed at 17, 55 or 230 μg/animal; ACP153, ACP155 and ACP156 were dosed at 55 or 230 μg/animal; ACP292 was dosed at 45 or 186 μg/animal. Body weights were taken at initiation and then biweekly to the end. Caliper measurements were taken biweekly to the end. Any adverse reactions were to be reported immediately. Any individual animal with a single observation of >than 30% body weight loss or three consecutive measurements of>25% body weight loss was euthanized. Any group with a mean body weight loss of >20% or >10% mortality stopped dosing; the group was not euthanized and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 45 days, whichever comes first. Responders were followed longer. When the endpoint was reached, the animals are to be euthanized. Results are shown in FIGS. 49A-49I.

### Example 27h: Treatment with ACP16, APC302 and ACP314

Agents and Treatment:

| | | | Formulation | | | |
|---|---|---|---|---|---|---|
| Gr. | N | Agent | dose | | Route | Schedule |
| 1^{#} | 12 | Vehicle | — | | ip | biwk x 2 |
| 2 | 9 | ACP16 | 55 | μg/animal | ip | biwk x 2 |
| 3 | 9 | ACP16 | 230 | μg/animal | ip | biwk x 2 |
| 4 | 9 | ACP302 | 33 | μg/animal | ip | biwk x 2 |
| 5 | 9 | ACP302 | 106 | μg/animal | ip | biwk x 2 |
| 6 | 9 | ACP302 | 442 | μg/animal | ip | biwk x 2 |
| 7 | 9 | ACP302 | 1,344 | μg/animal | ip | biwk x 2 |
| 8 | 9 | ACP314 | 21 | μg/animal | ip | biwk x 2 |
| 9 | 9 | ACP314 | 68 | μg/animal | ip | biwk x 2 |
| 10 | 9 | ACP314 | 283 | μg/animal | ip | biwk x 2 |
| 11 | 9 | ACP314 | 861 | μg/animal | ip | biwk x 2 |

### Procedures:

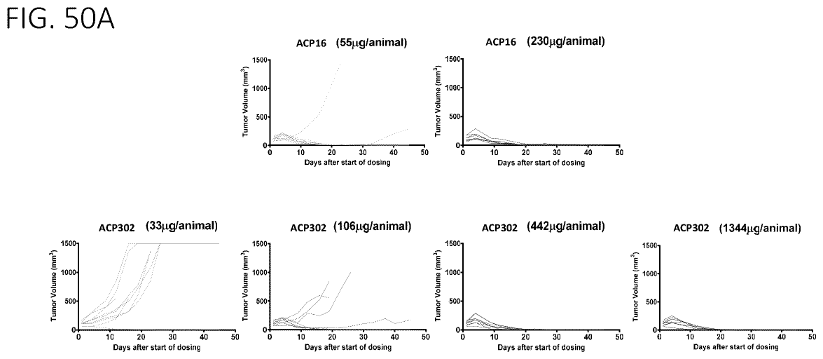

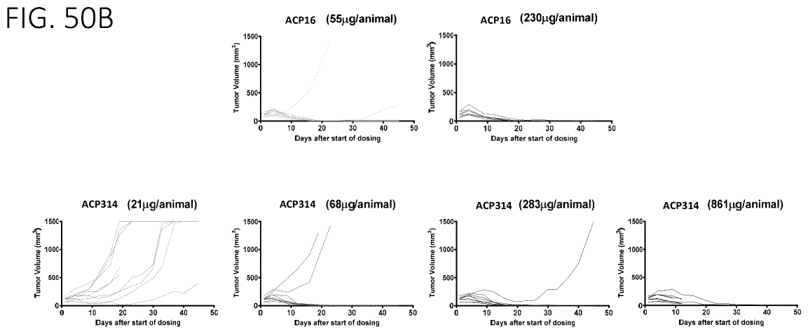

Mice were anaesthetized with isoflurane for implant of cells to reduce the ulcerations. CR female C57BL/6 mice were set up with 5×10⁵ MC38 tumor cells in 0% Matrigel sc in flank. Cell Injection Volume was 0.1 mL/mouse. Mouse age at start date was 8 to 12 weeks. Pair matches were performed when tumors reach an average size of 100-150 mm³ and begin treatment. ACP16 was dosed at 55 or 230 μg/animal; ACP302 was dosed at 33, 106, 442 or 1344 ug/animal; ACP314 was dosed at 21,68, 283 or 861 μg/animal. Body weights were taken at initiation and then biweekly to the end. Caliper measurements were taken biweekly to the end. Any adverse reactions were to be reported immediately. Any individual animal with a single observation of >than 30% body weight loss or three consecutive measurements of >25% body weight loss was euthanized. Any group with a mean body weight loss of >20% or >10% mortality stopped dosing; the group was not euthanized and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 45 days, whichever comes first. Responders were followed longer. When the endpoint was reached, the animals are to be euthanized. Results are shown in FIG. 50A and FIG. 50B.

### Example 27i: Treatment with ACP339

Agents and Treatment:

| Gr. | N | Agent | Formulation dose | | Route | Schedule |
|---|---|---|---|---|---|---|
| 1^{#} | 12 | Vehicle | — | | ip | biwk x 2 |
| 2 | 9 | ACP339 | 55 | μg/animal | ip | biwk x 2 |
| 3 | 9 | ACP339 | 230 | μg/animal | ip | biwk x 2 |
| 4 | 9 | ACP339 | 700 | μg/animal | ip | biwk x 2 |

### Procedures:

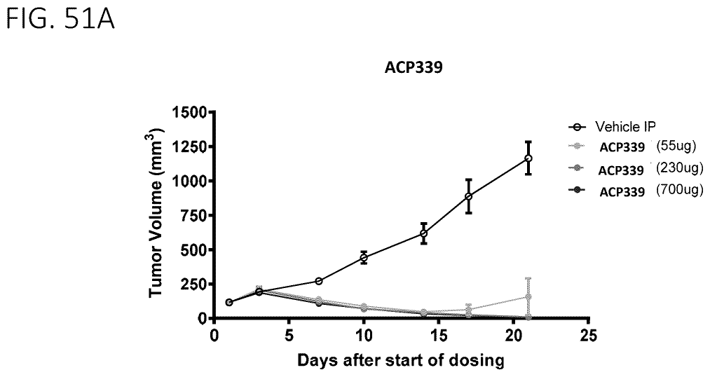

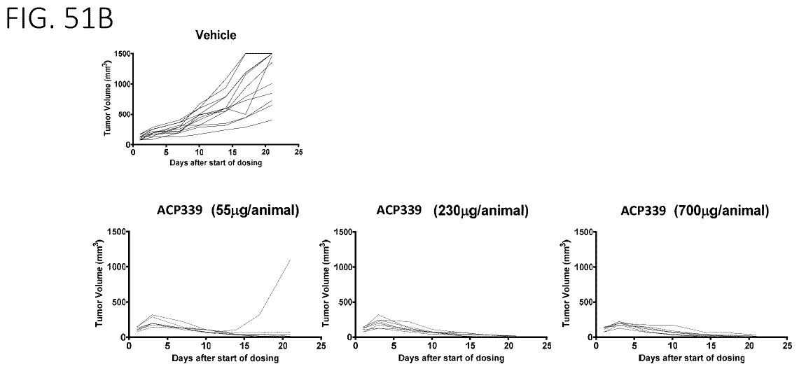

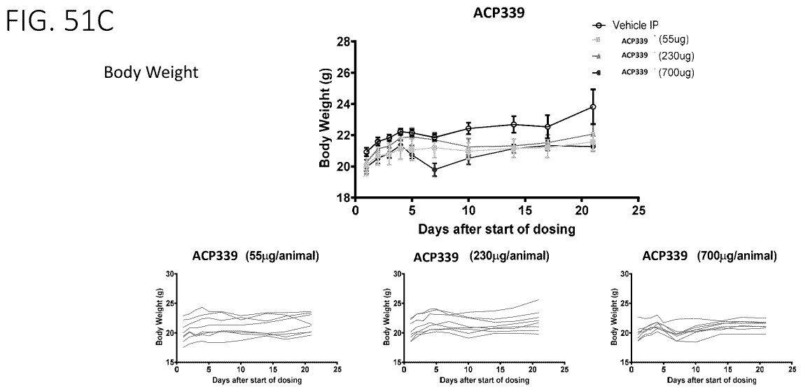

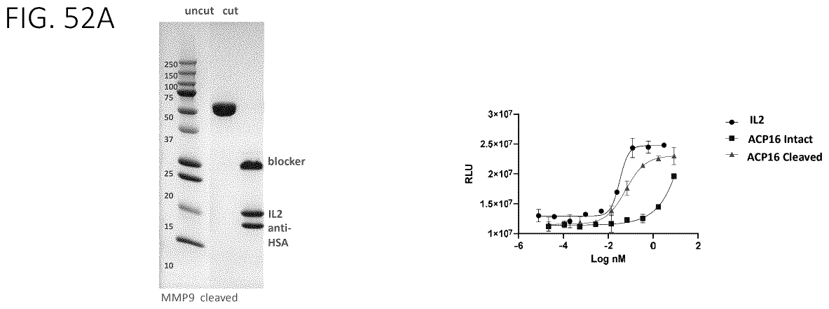

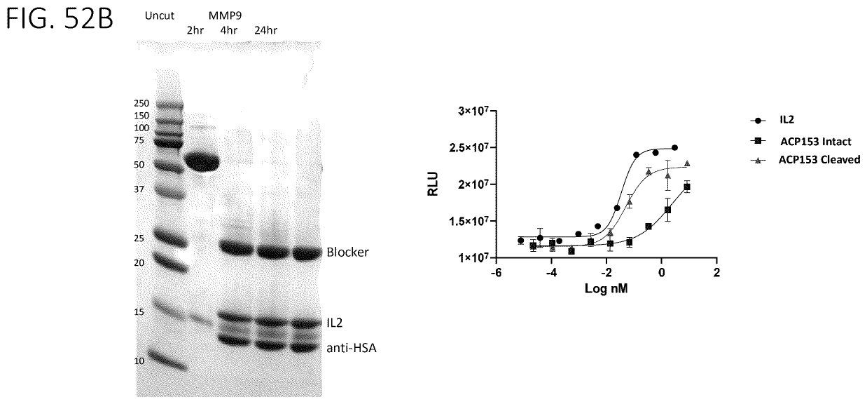

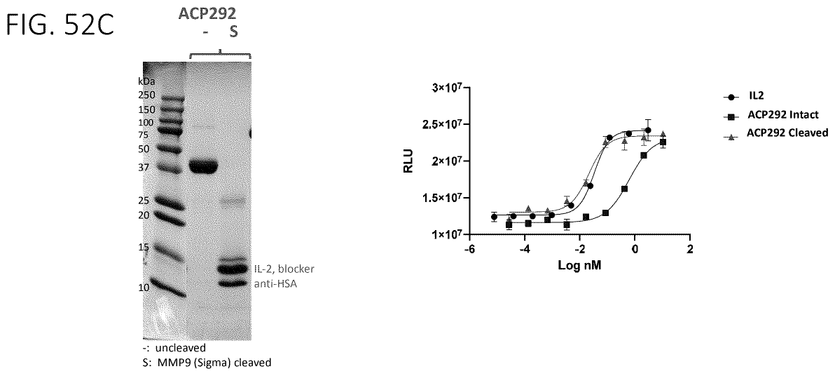

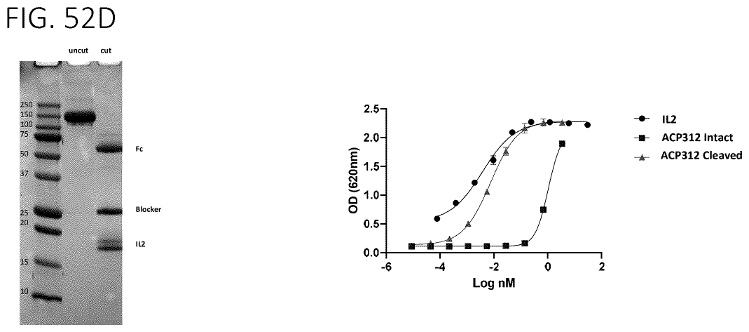

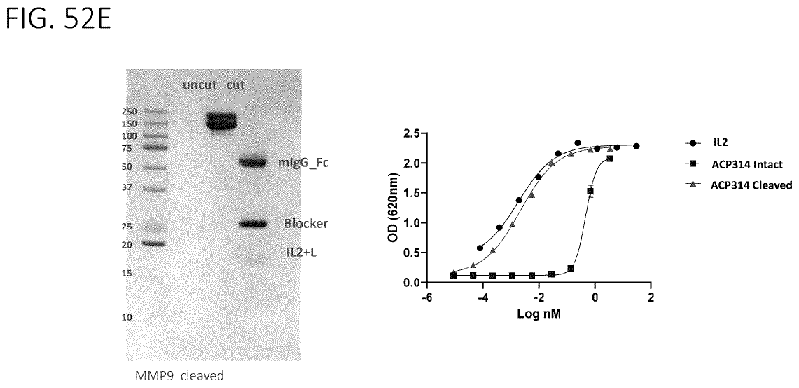

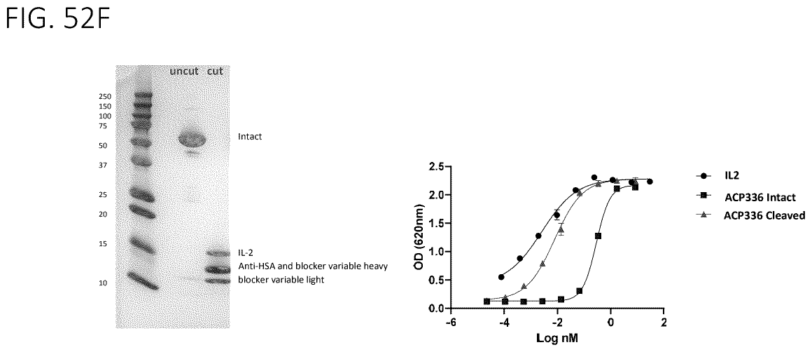

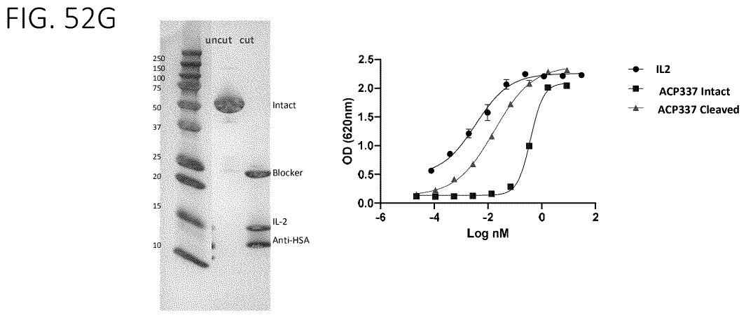

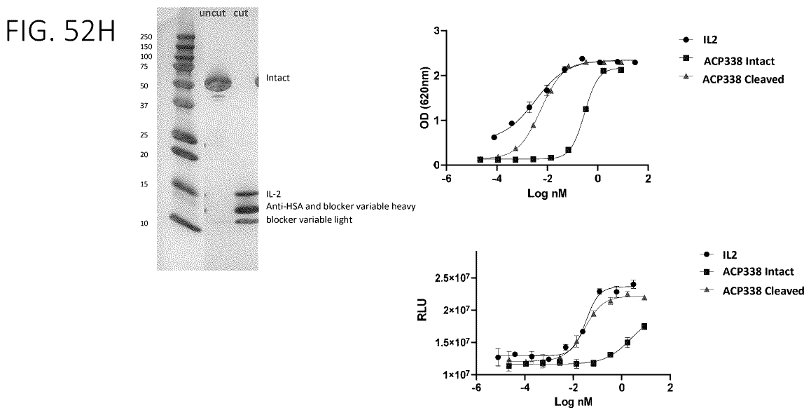

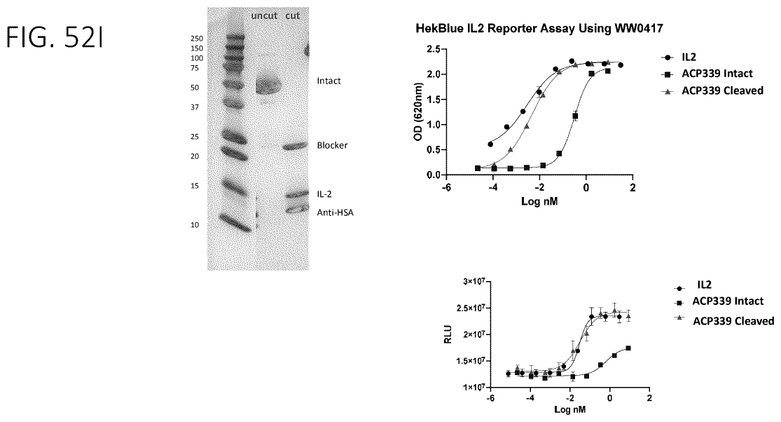

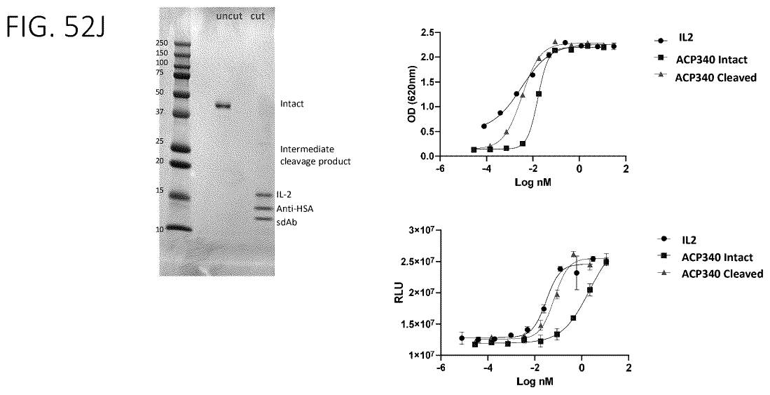

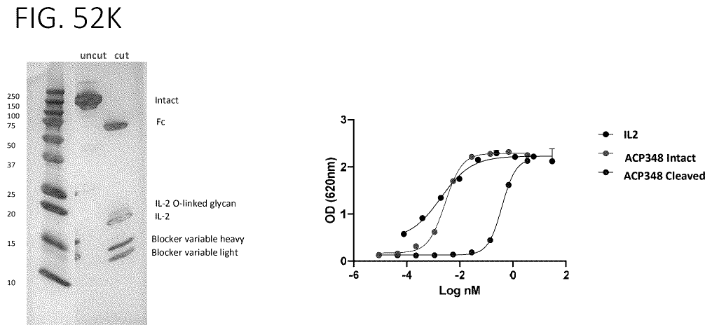

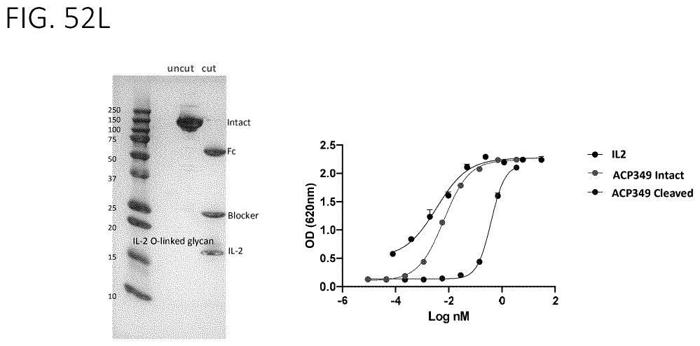

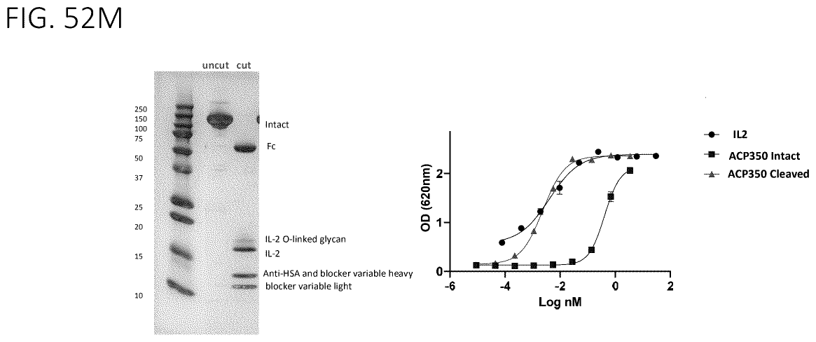

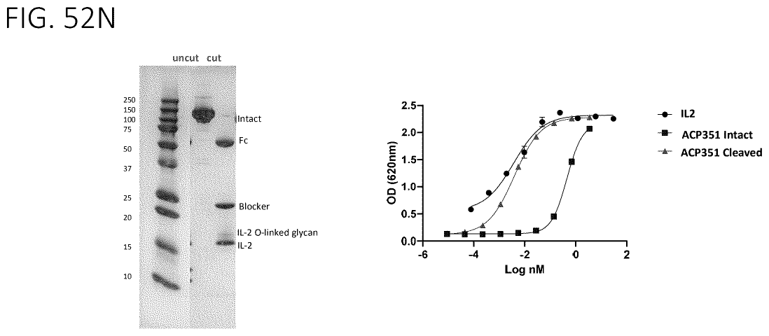

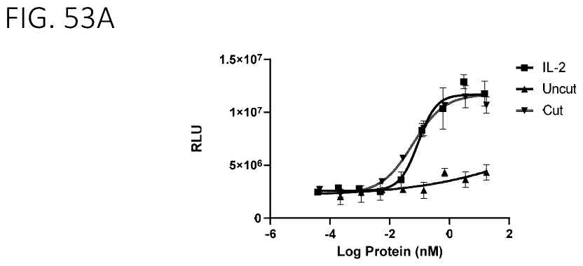

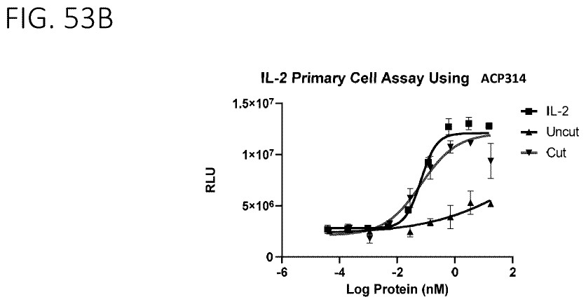

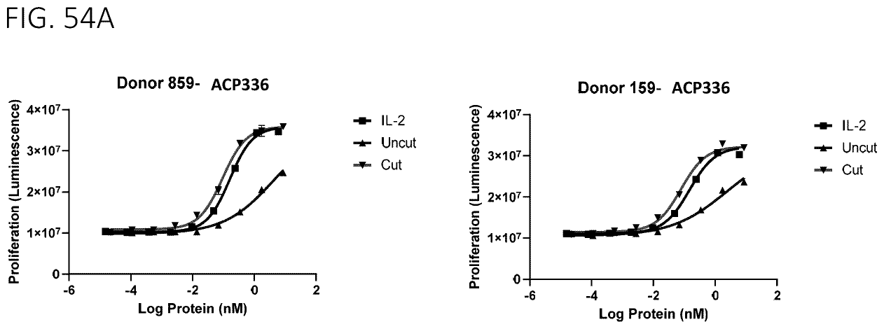

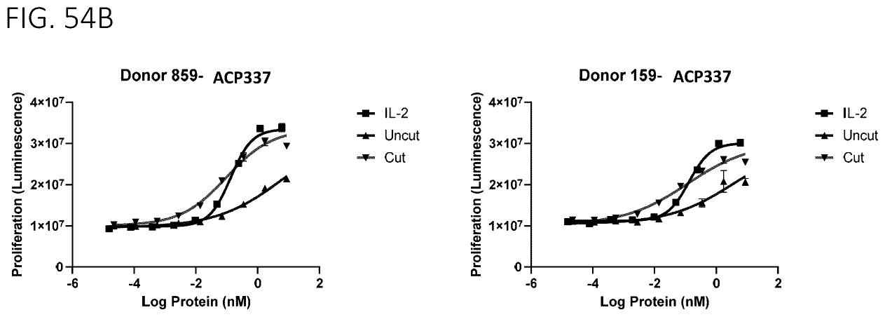

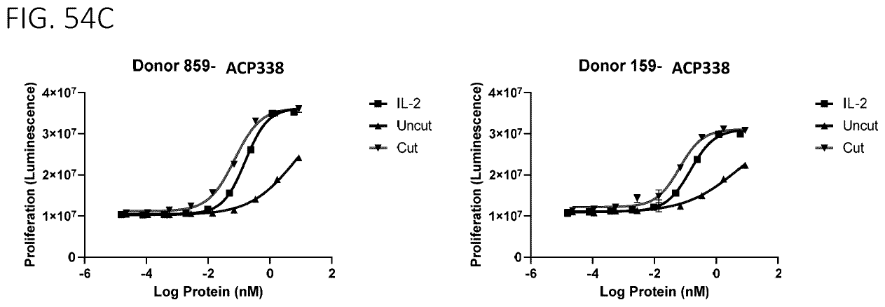

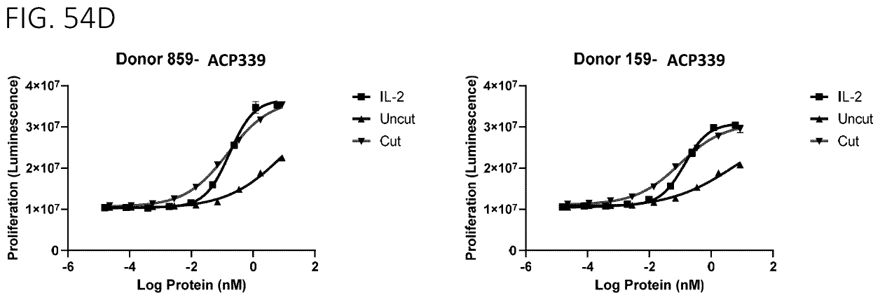

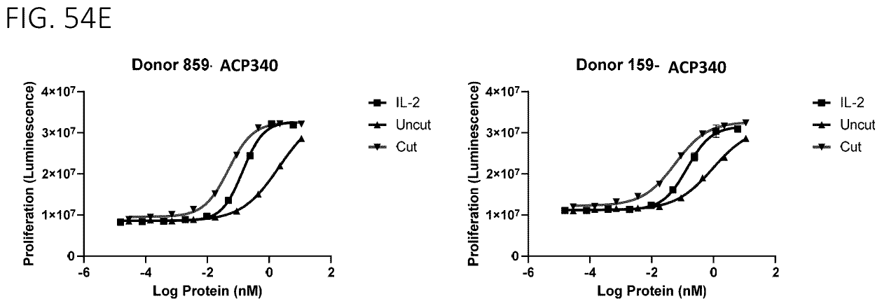

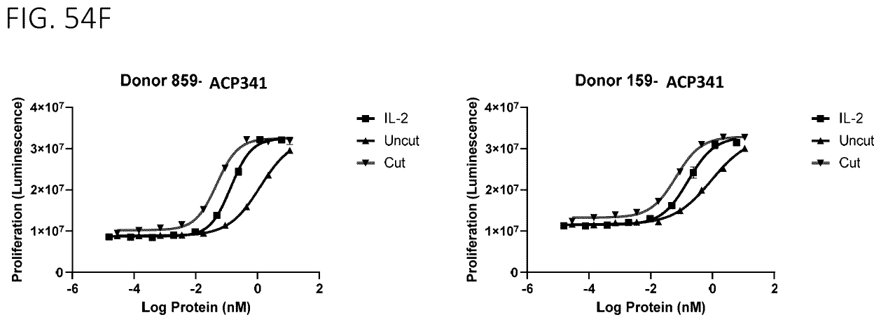

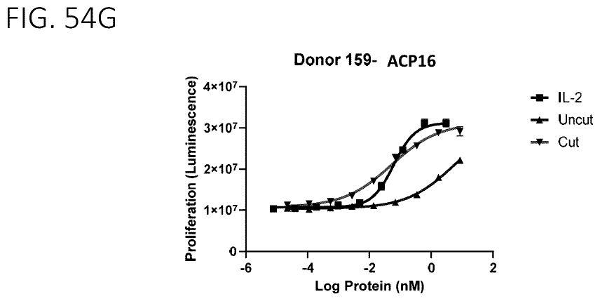

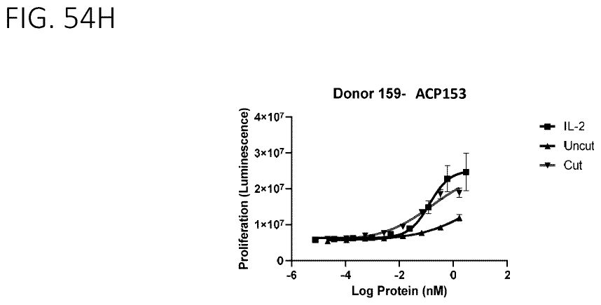

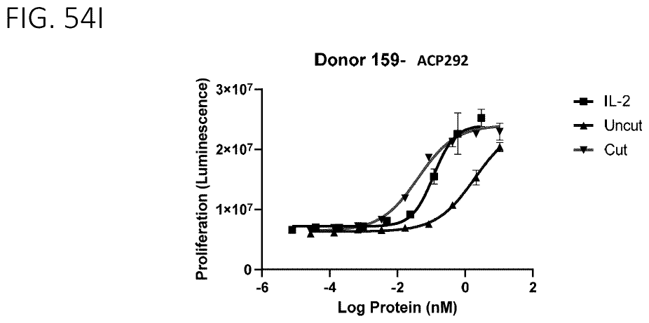

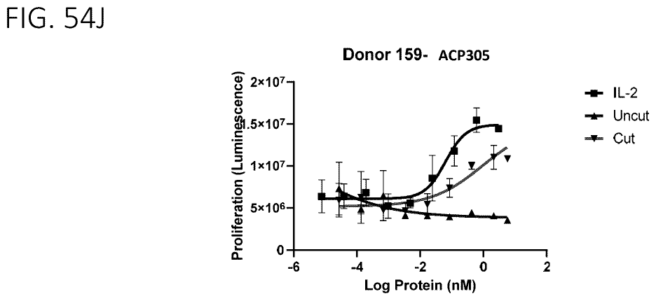

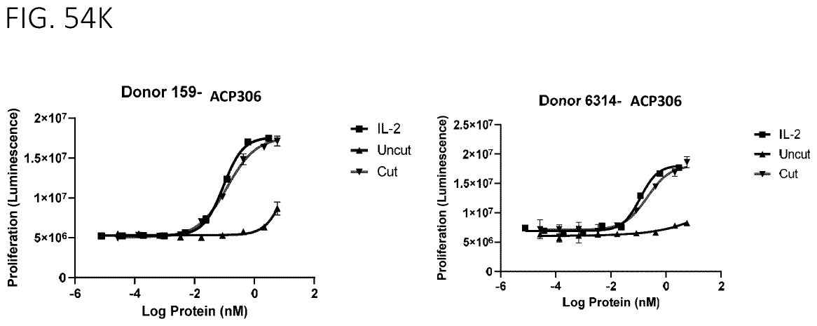

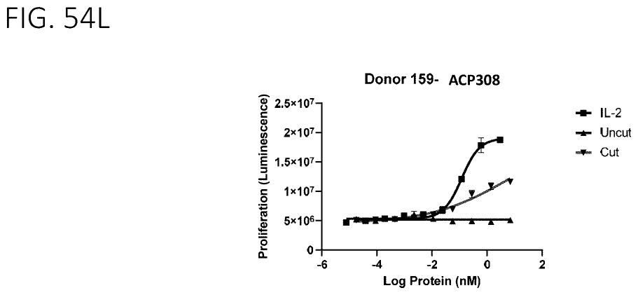

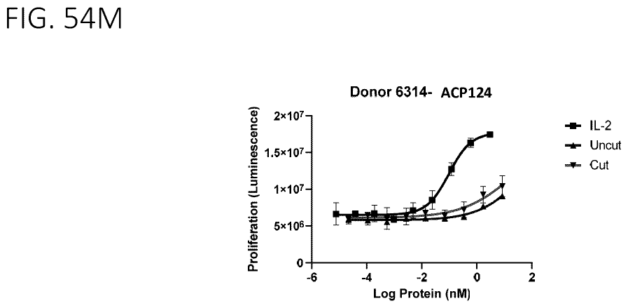

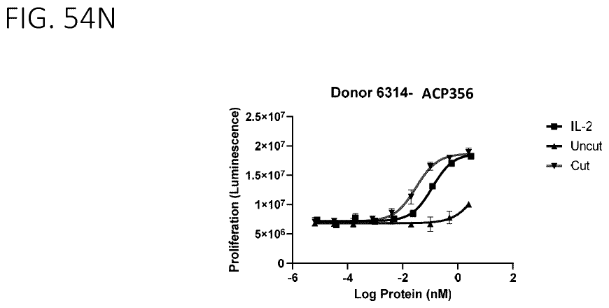

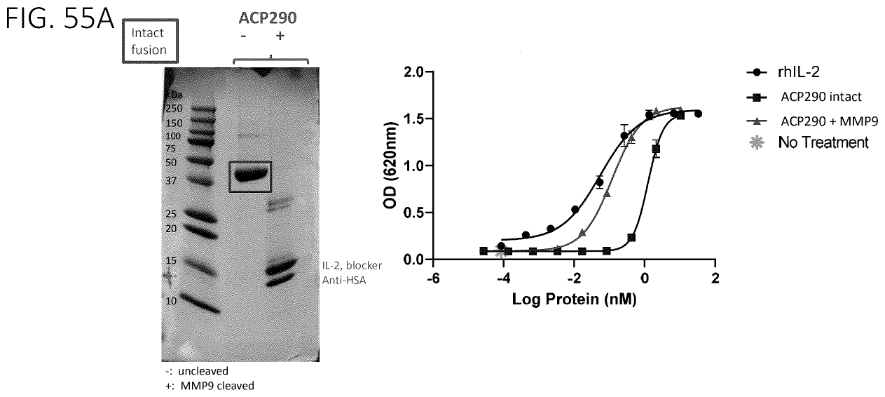

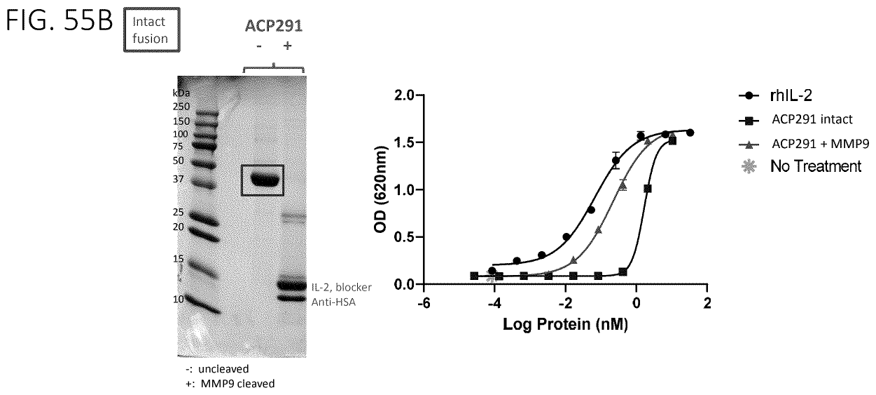

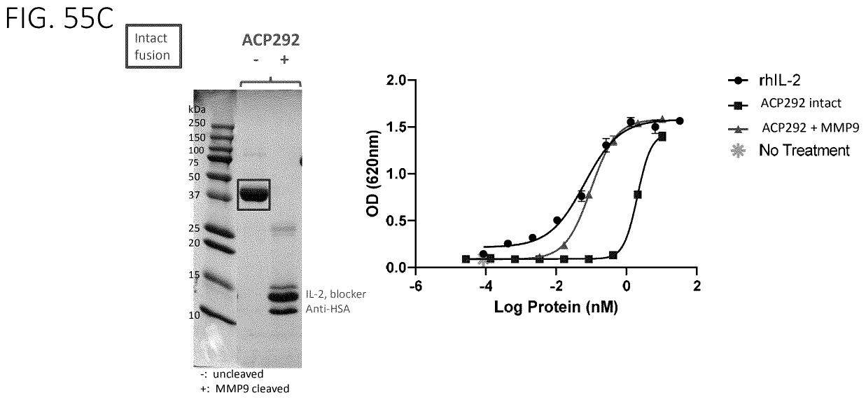

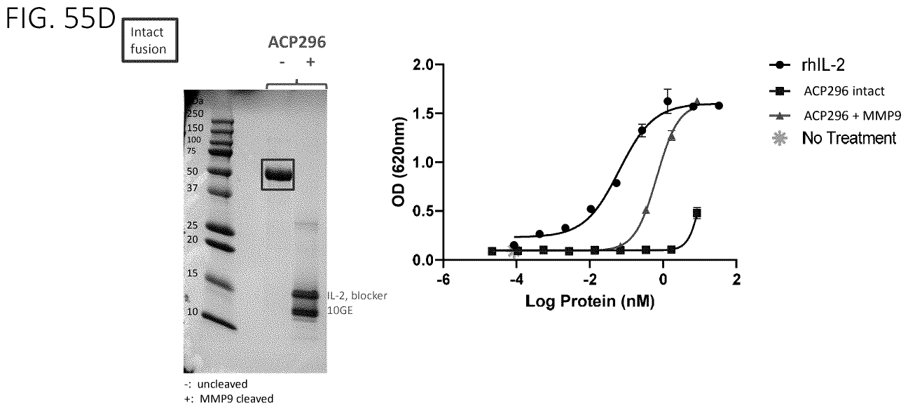

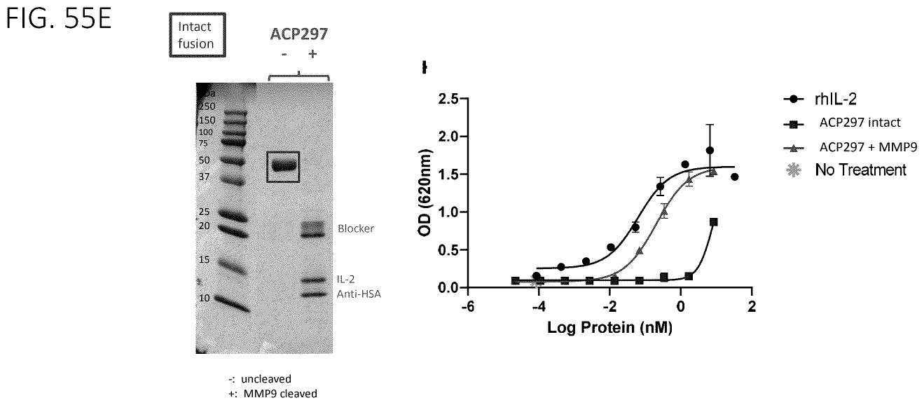

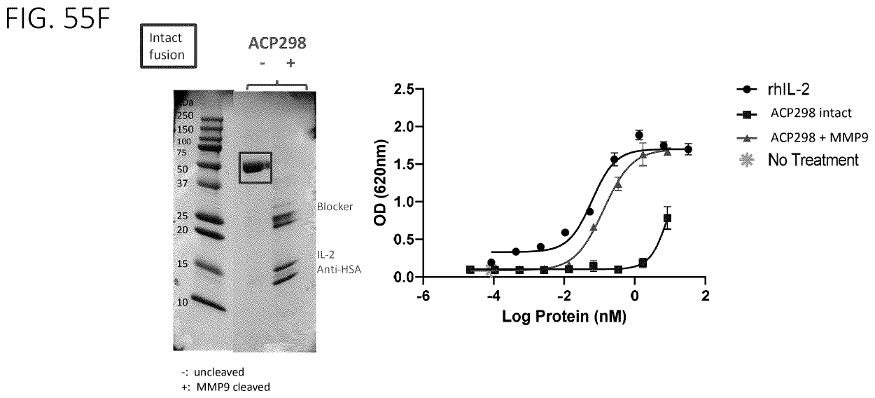

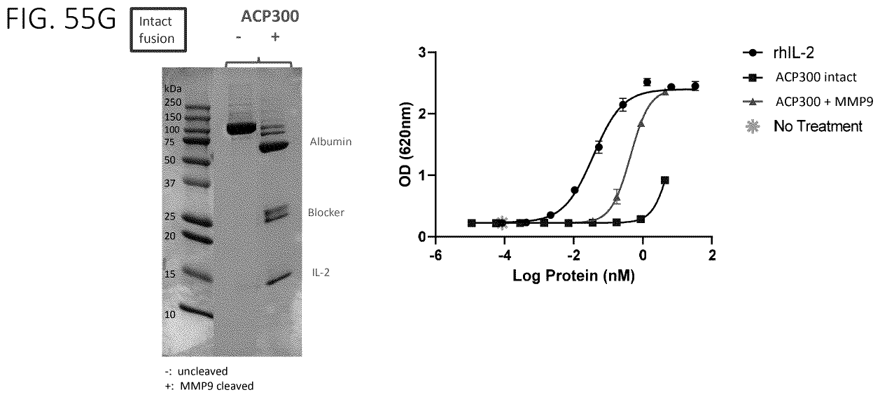

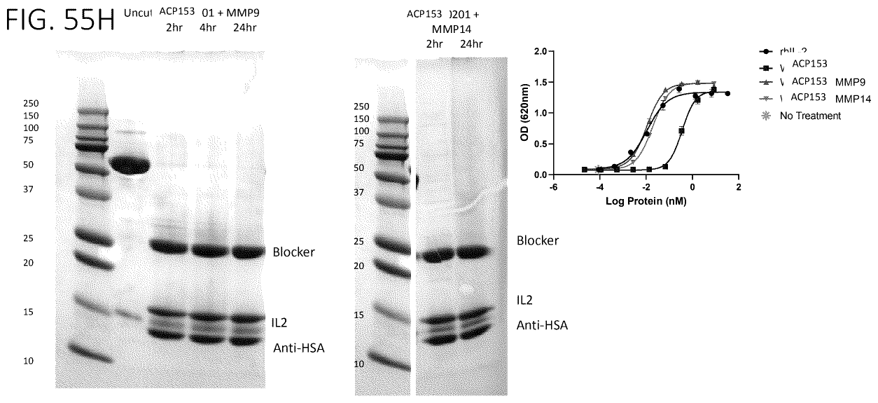

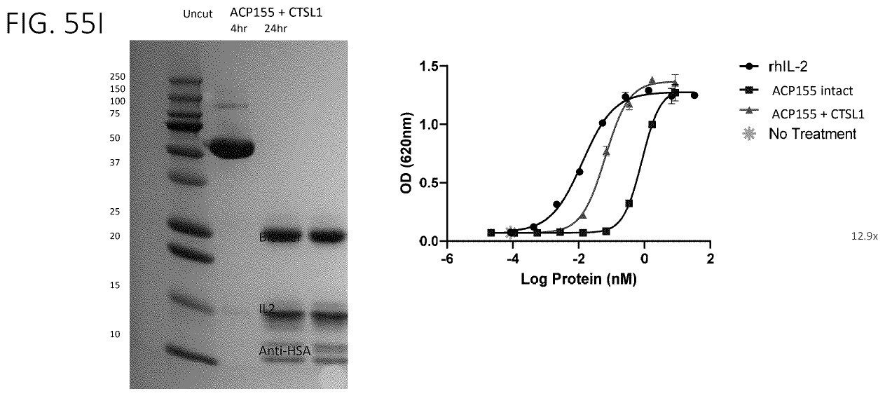

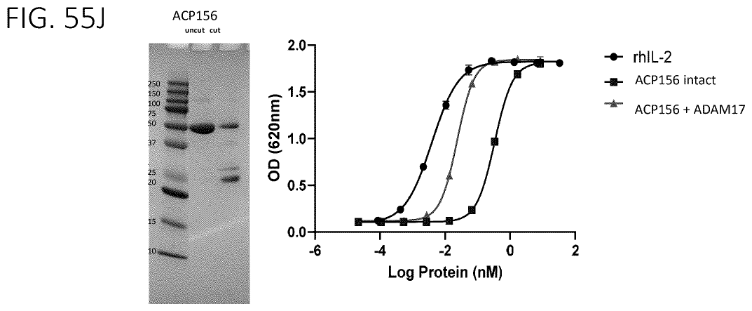

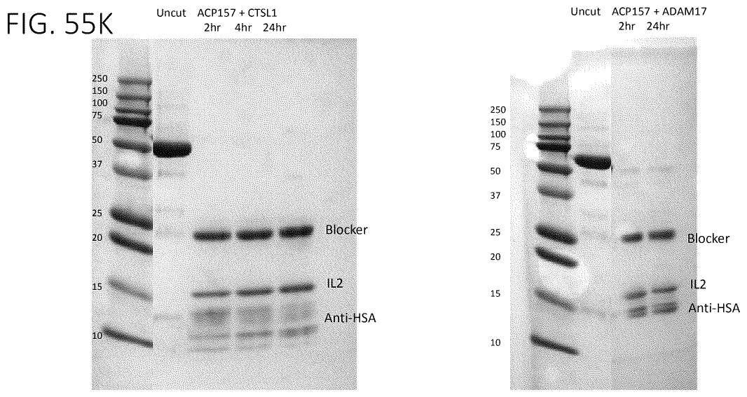

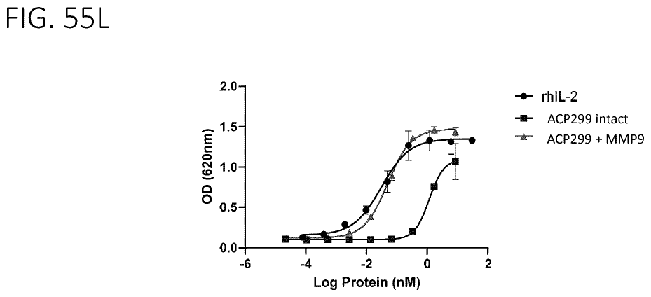

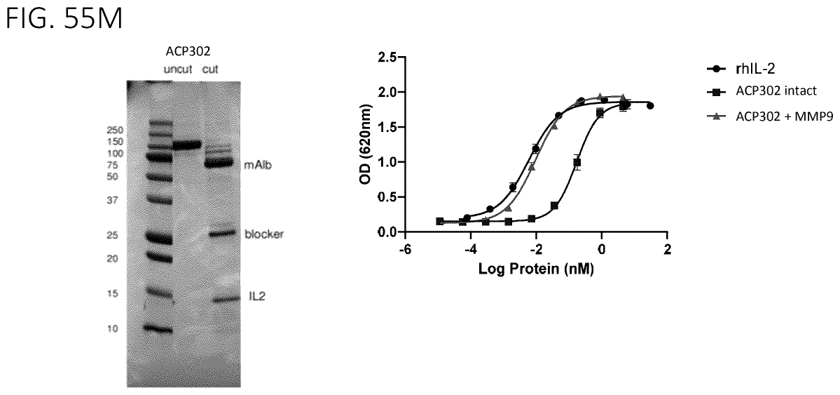

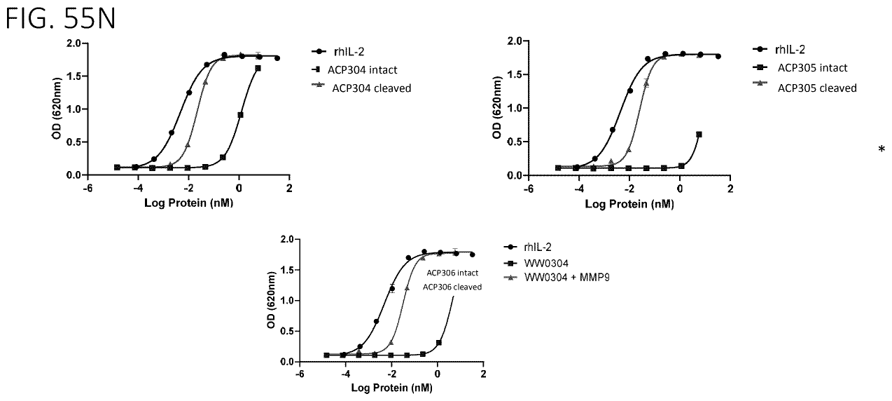

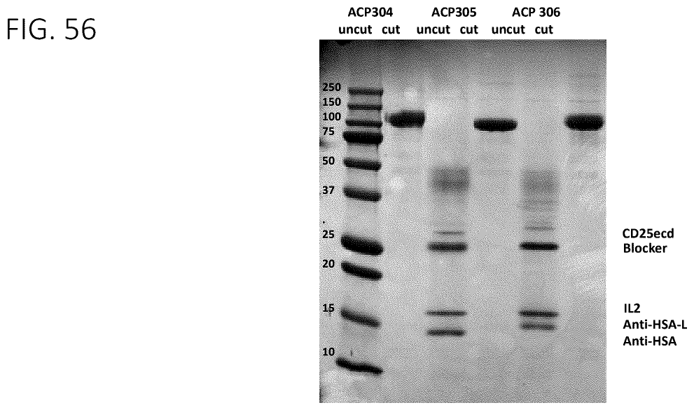

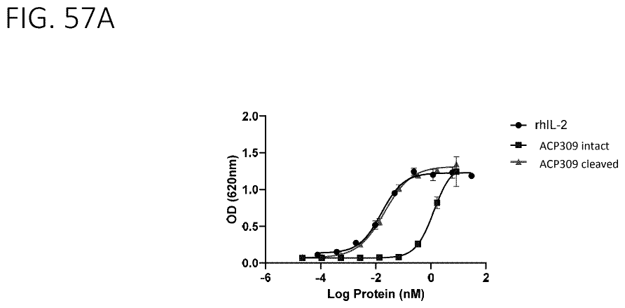

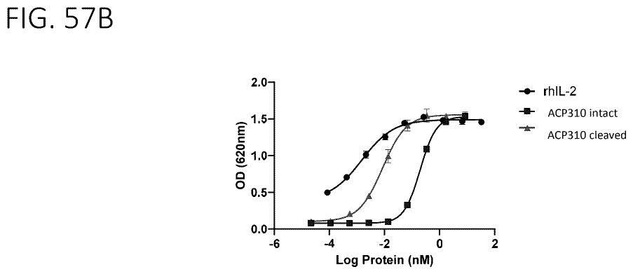

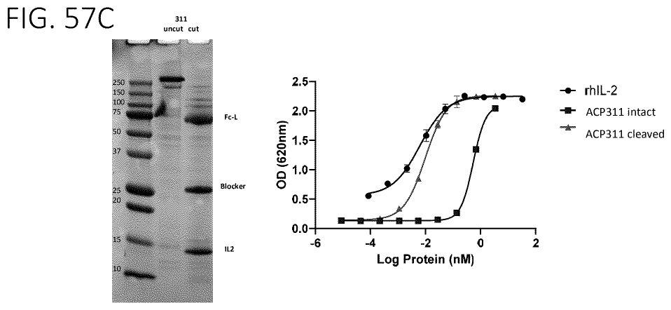

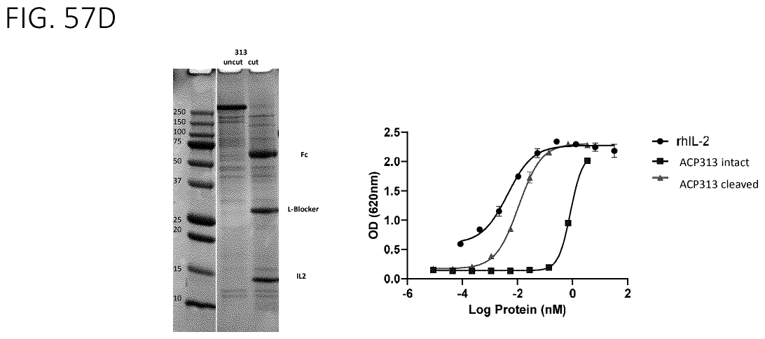

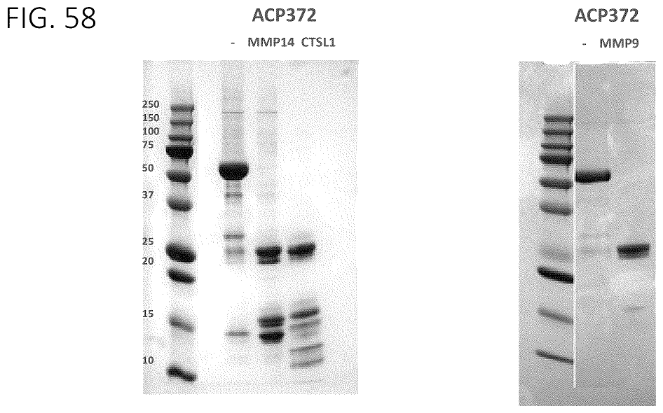

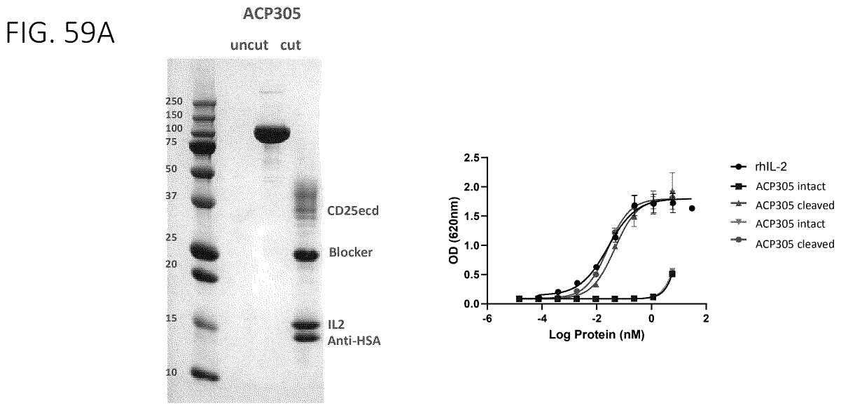

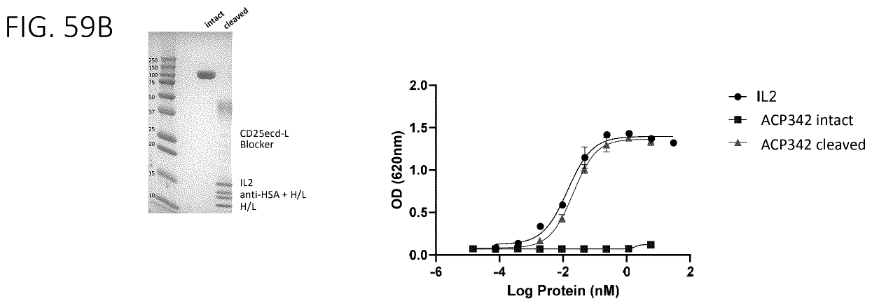

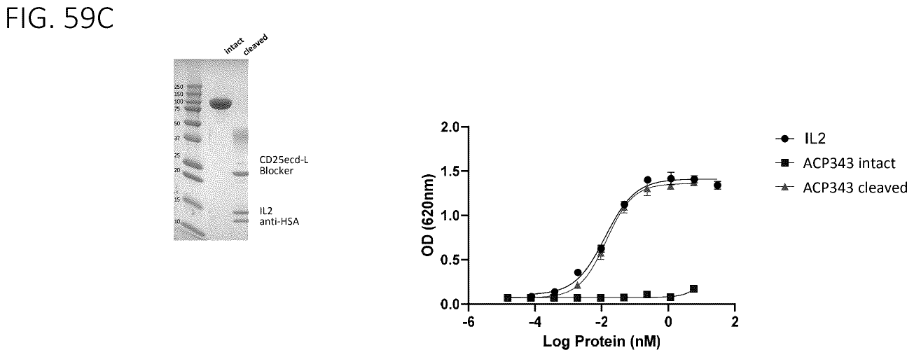

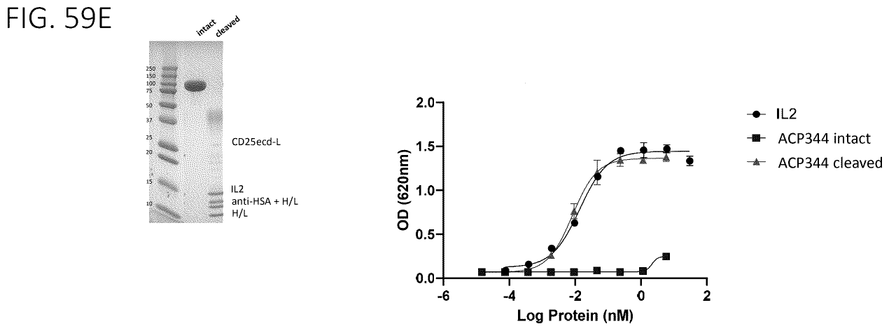

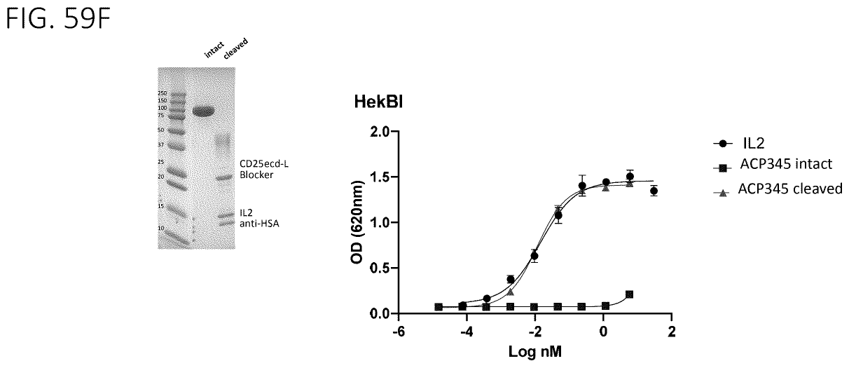

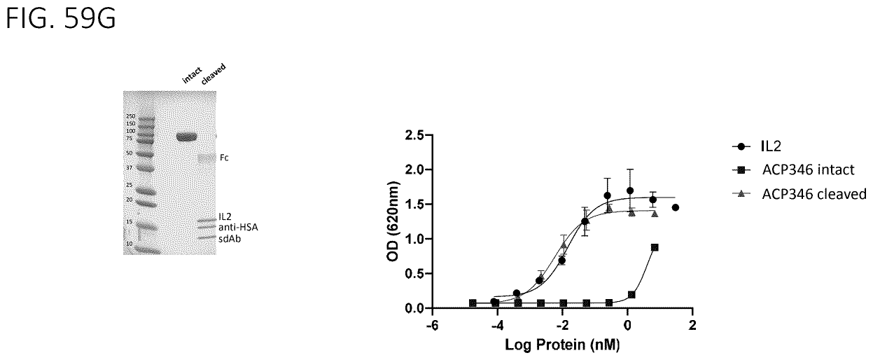

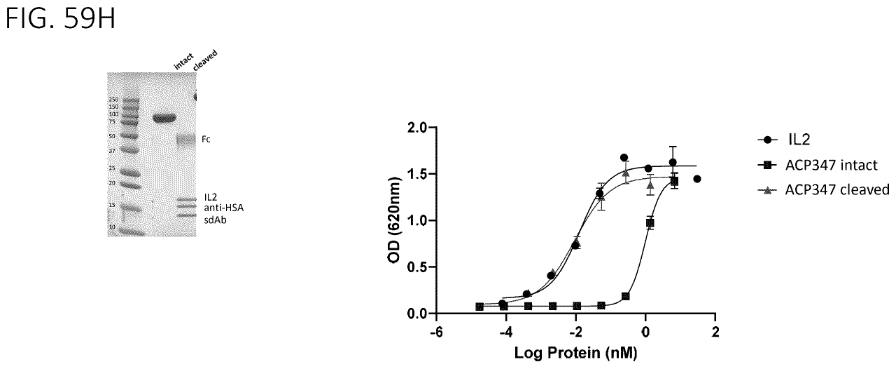

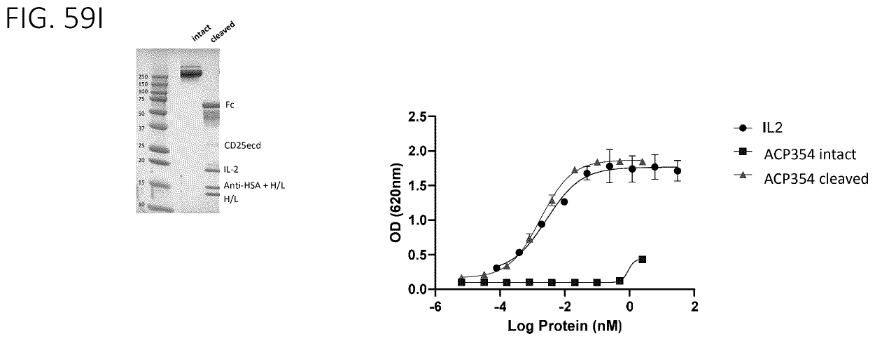

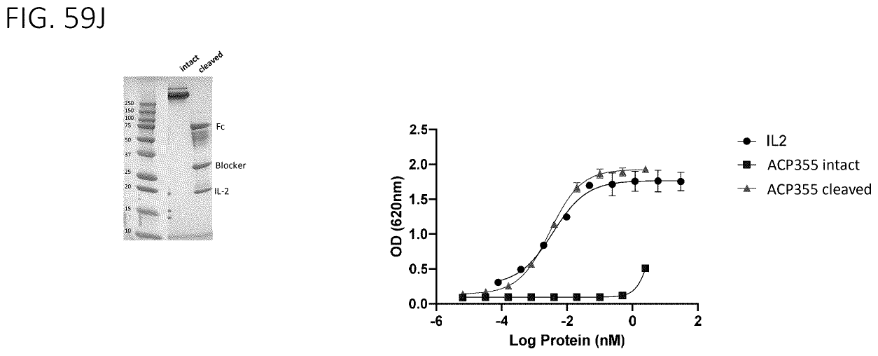

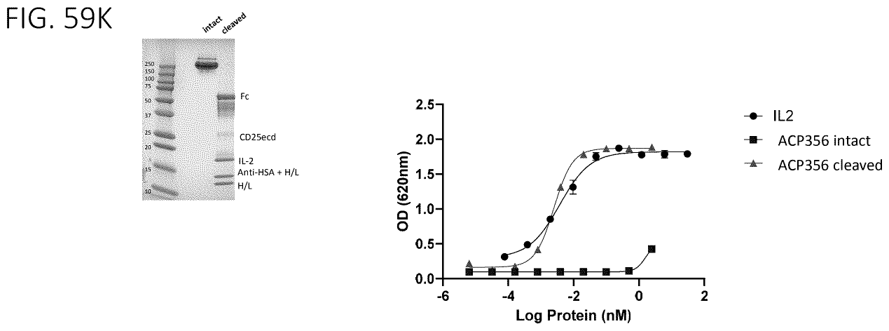

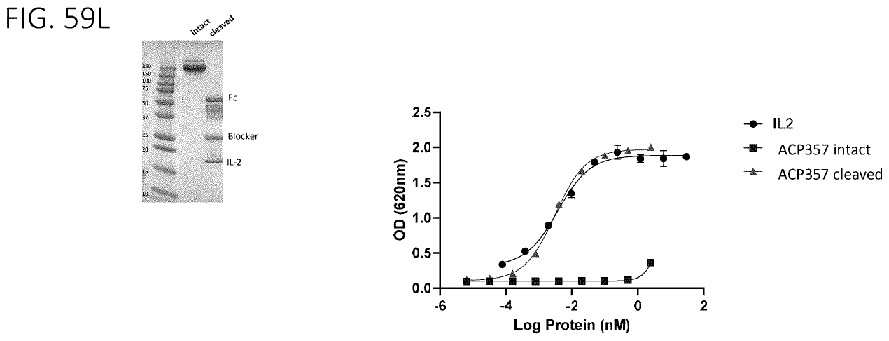

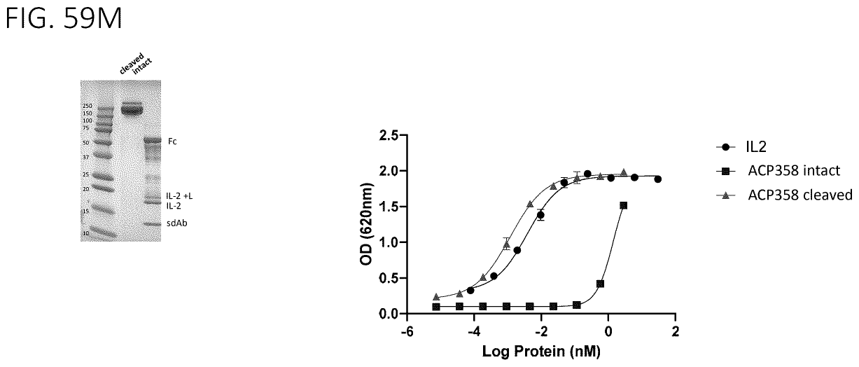

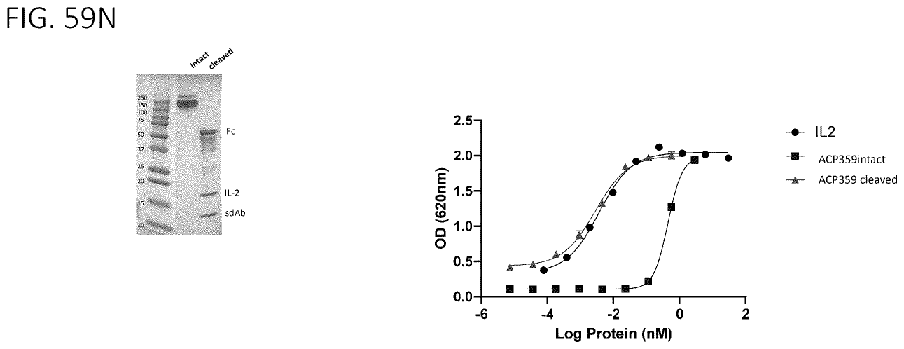

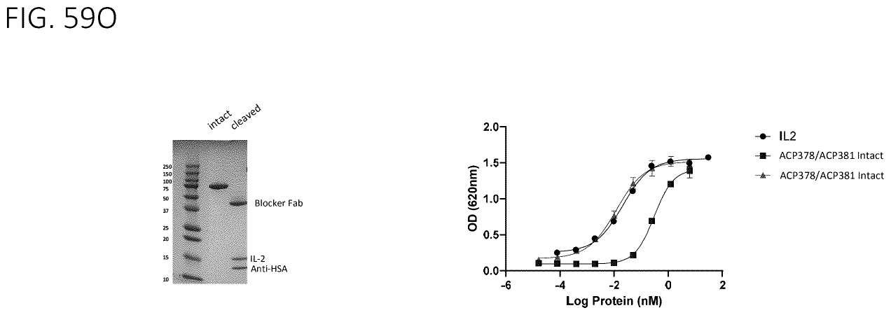

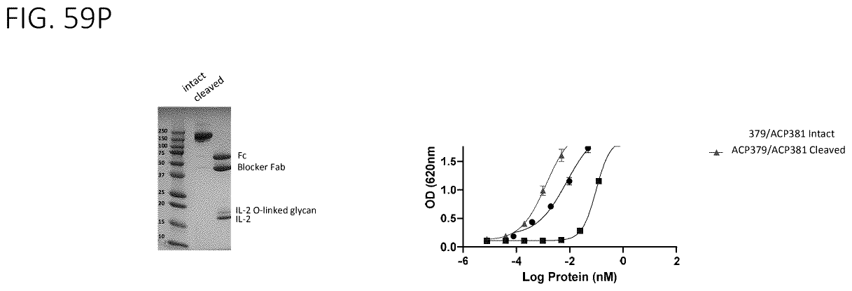

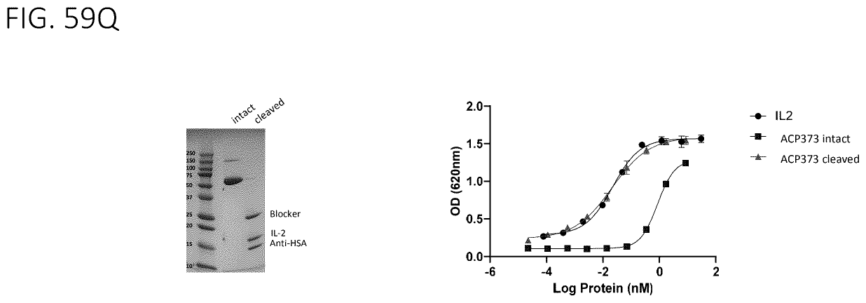

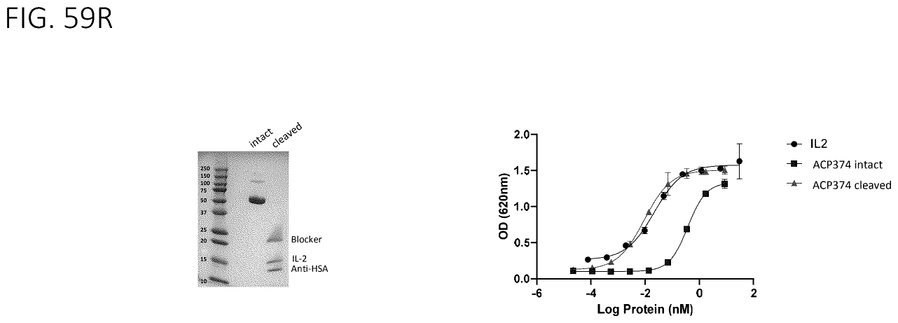

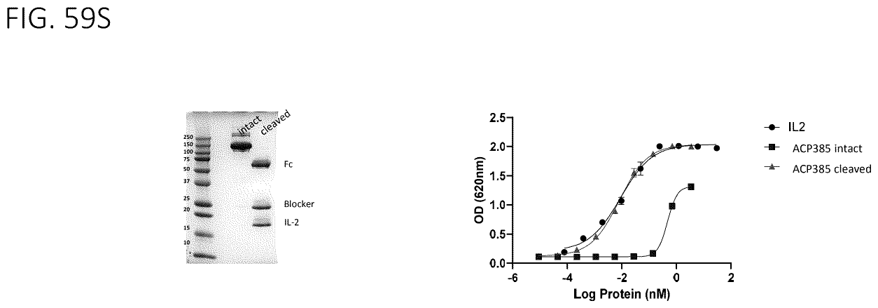

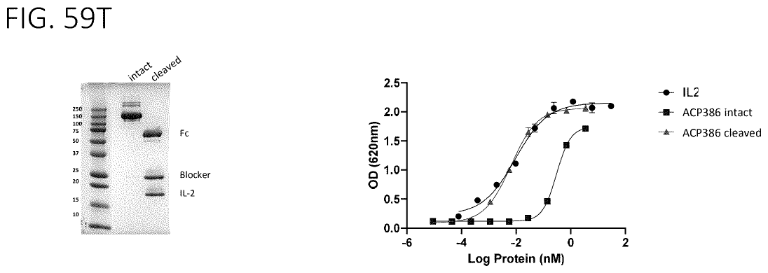

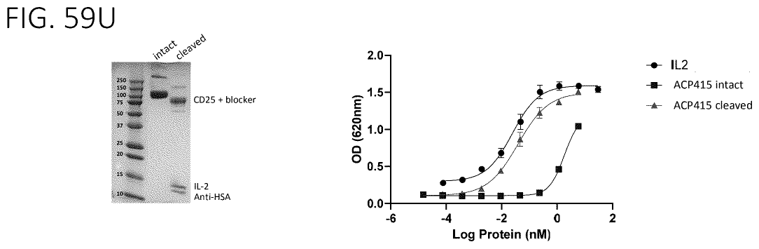

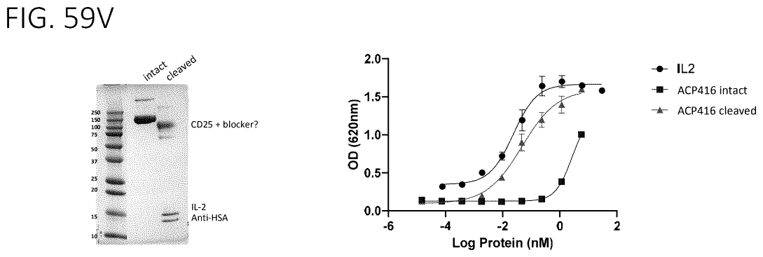

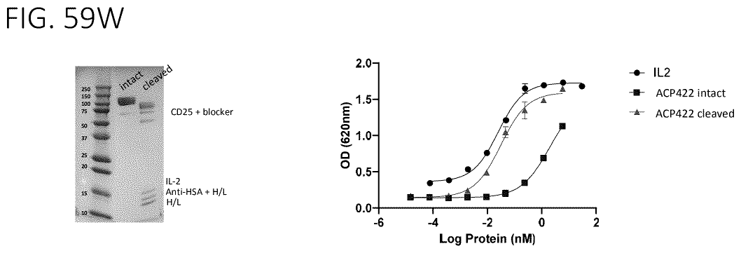

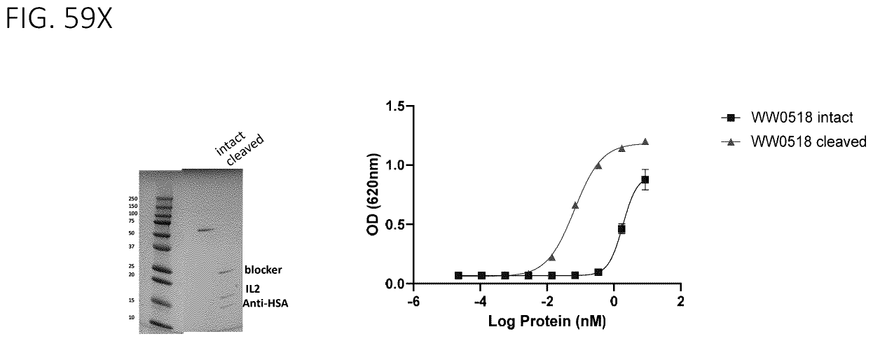

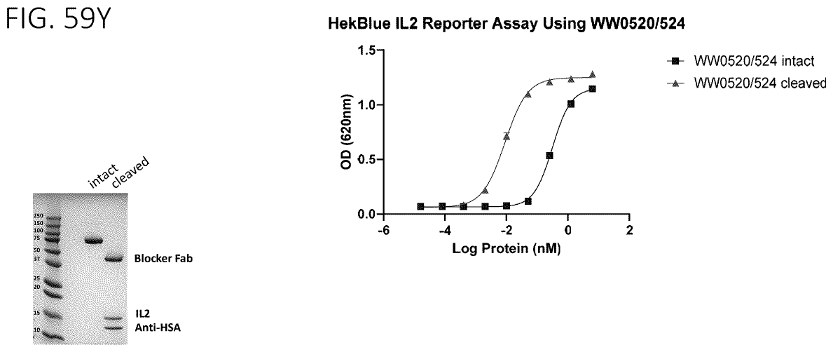

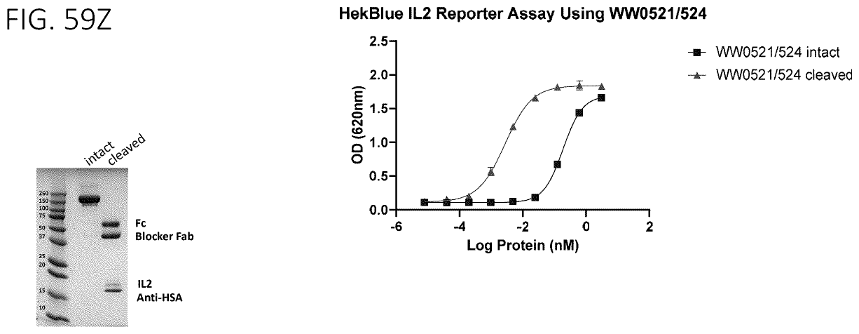

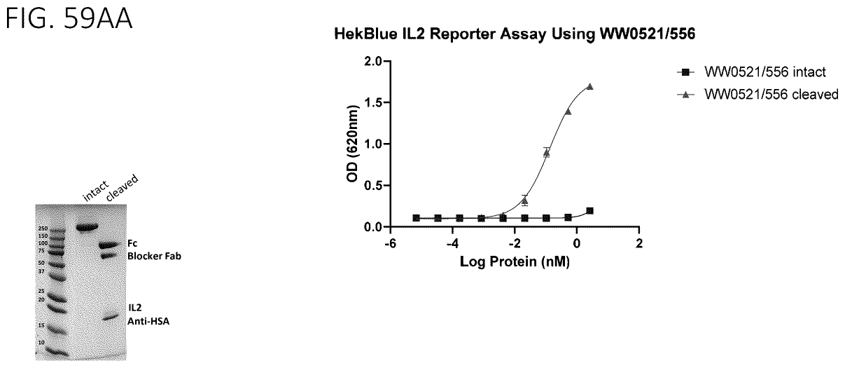

Mice were anaesthetized with isoflurane for implant of cells to reduce the ulcerations. CR female C57BL/6 mice were set up with 5×10⁵ MC38 tumor cells in 0% Matrigel sc in flank. Cell Injection Volume was 0.1 mL/mouse. Mouse age at start date was 8 to 12 weeks. Pair matches were performed when tumors reach an average size of 100-150 mm³ and begin treatment. ACP339 was dosed at 55, 230 or 700 μg/animal. Body weights were taken at initiation and then biweekly to the end. Caliper measurements were taken biweekly to the end. Any adverse reactions were to be reported immediately. Any individual animal with a single observation of >than 30% body weight loss or three consecutive measurements of >25% body weight loss was euthanized. Any group with a mean body weight loss of >20% or >10% mortality stopped dosing; the group was not euthanized and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 45 days, whichever comes first. Responders were followed longer. When the endpoint was reached, the animals are to be euthanized. Results are shown in FIGS. 51A-51C.

### Example 28: CT26 experiments

The CT26 cell line, a rapidly growing colon adenocarcinoma cell line that expresses MMP9 in vitro, was used. Using this tumor model, the ability of fusion proteins to affect tumor growth was examined.

### Example 28a: Treatment with ACP16 alone or in combination with anti-PD1 antibody

Agents and Treatment:

| | | | Formulation | | |
|---|---|---|---|---|---|
| Gr. | N | Agent | dose | Route | Schedule |
| 1^{#} | 12 | vehicle 1// | na// | ip//ip | days 1, 4, 8, 11// |
| | | vehicle 2 | na | | days 3, 6, 10, 13 |
| 2 | 10 | vehicle 1// | na// | ip//ip | days 1, 4, 8, 11// |
| | | ACP16 | 70 μg/animal | | days 3, 6, 10, 13 |
| 3 | 10 | vehicle 1// | na// | ip//ip | days 1, 4, 8, 11// |
| | | ACP16 | 232 μg/animal | | days 3, 6, 10, 13 |
| 4 | 10 | vehicle 1// | na// | ip//ip | days 1, 4, 8, 11// |
| | | ACP16 | 500 μg/animal | | days 3, 6, 10, 13 |
| 5 | 10 | anti-PD-1 RMP1-14// | 200 μg/animal// | ip//ip | days 1, 4, 8, 11// |
| | | vehicle 2 | na | | days 3, 6, 10, 13 |
| 6 | 10 | anti-PD-1 RMP1-14// | 200 μg/animal// | ip//ip | days 1, 4, 8, 11// |
| | | ACP16 | 70 μg/animal | | days 3, 6, 10, 13 |
| 7 | 10 | anti-PD-1 RMP1-14// | 200 μg/animal// | ip//ip | days 1, 4, 8, 11// |
| | | ACP16 | 232 μg/animal | | days 3, 6, 10, 13 |
| 8 | 10 | anti-PD-1 RMP1-14// | 200 μg/animal// | ip//ip | days 1, 4, 8, 11// |
| | | ACP 16 | 500 μg/animal | | days 3, 6, 10, 13 |
| 9 | 10 | vehicle 1// | na// | ip//ip | days 1, 4, 8, 11// |
| | | IL-2 | 12 μg/animal | | bid x 5 first day 1 |
| | | | | | dose per week x 2 |
| 10 | 10 | anti-PD-1 RMP1-14// | 200 μg/animal// | ip//ip | days 1, 4, 8, 11// |
| | | IL-2 | 12 μg/animal | | bid x 5 first day 1 |
| | | | | | dose per week x 2 |

### Procedures:

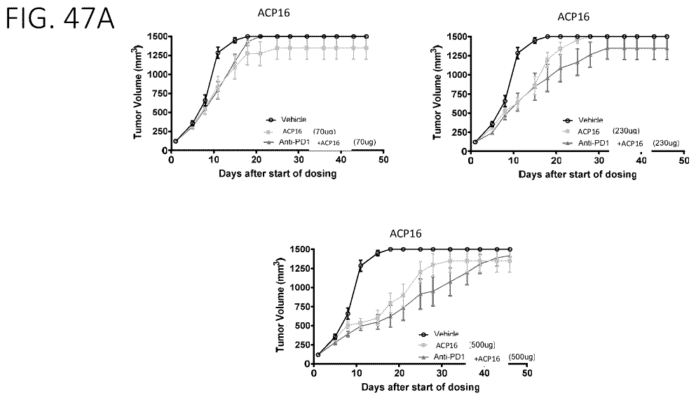

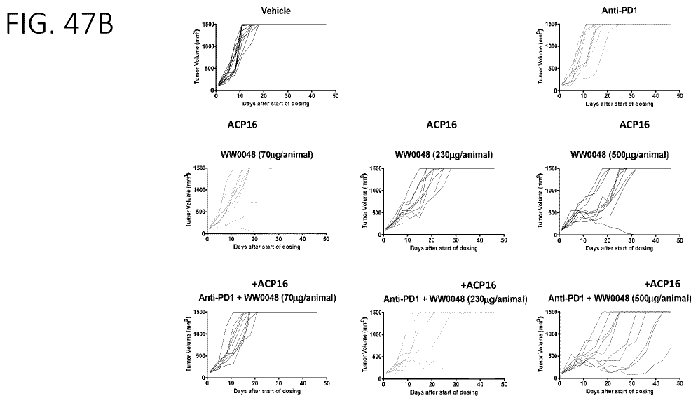

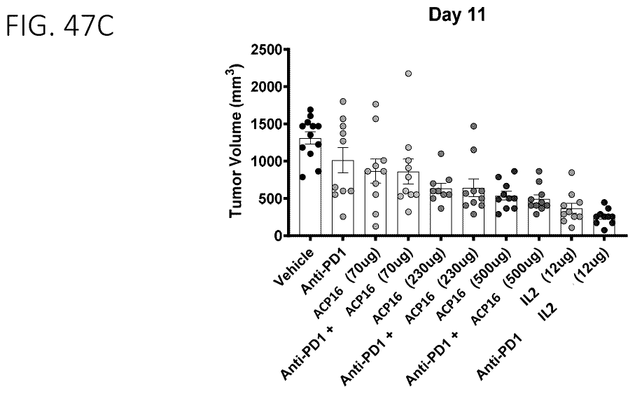

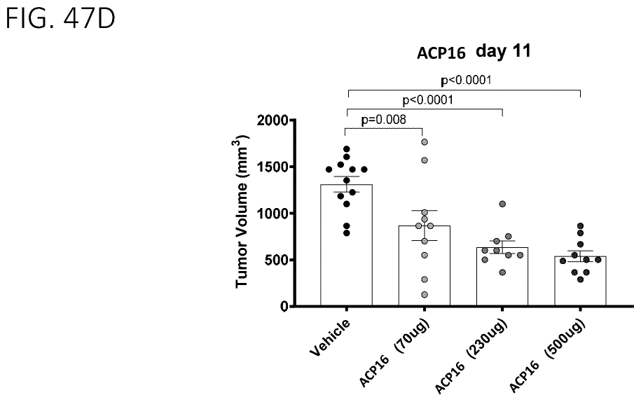

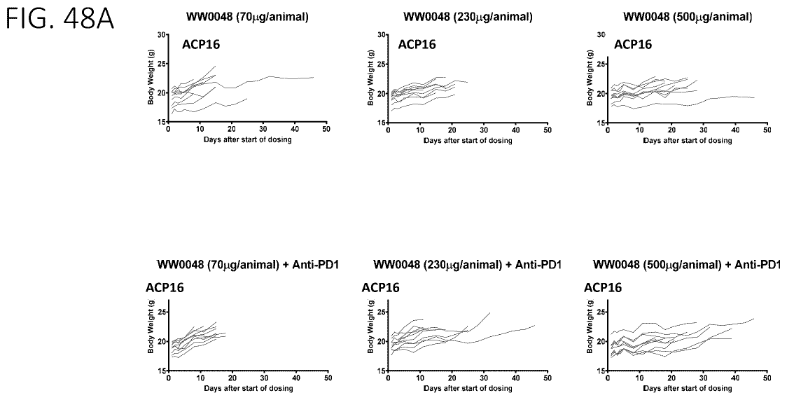

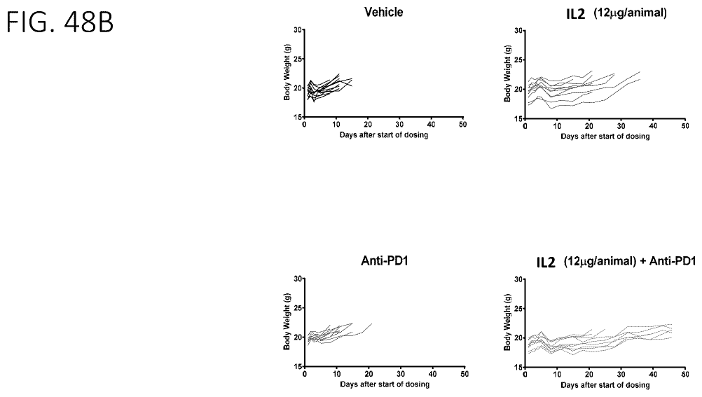

Mice were anaesthetized with isoflurane for implant of cells to reduce the ulcerations. CR female BALB/c mice were set up with 3×10⁵ CT26 tumor cells in 0% Matrigel sc in flank. Cell Injection Volume was 0.1 mL/mouse. Mouse age at start date was 8 to 12 weeks. Pair matches were performed when tumors reach an average size of 100-150 mm³ and begin treatment. ACP16 was dosed at 70, 230 or 500 jig/animal with or without anti-PD−1 antibody (RMP1-14) at 200 jig/animal. Body weights were taken at initiation and then biweekly to the end. Caliper measurements were taken biweekly to the end. Any adverse reactions were to be reported immediately. Any individual animal with a single observation of >than 30% body weight loss or three consecutive measurements of >25% body weight loss was euthanized. Any group with a mean body weight loss of >20% or >10% mortality stopped dosing; the group was not euthanized and recovery is allowed. Within a group with >20% weight loss, individuals hitting the individual body weight loss endpoint were euthanized. If the group treatment related body weight loss is recovered to within 10% of the original weights, dosing resumed at a lower dose or less frequent dosing schedule. Exceptions to non-treatment body weight % recovery were allowed on a case-by-case basis. Endpoint was tumor growth delay (TGD). Animals were monitored individually. The endpoint of the experiment was a tumor volume of 1500 mm³ or 45 days, whichever comes first. Responders were followed longer. When the endpoint was reached, the animals are to be euthanized. Results are shown in FIGS. 47A-47D and FIGS. 48A-48B.

### Example 29. Human Tblast Assay

Pre-stimulated T cells (T-blasts) were used to assess the activity of inducible IL-2 fusion proteins. T-Blasts were induced from human PBMCs with a 3-day incubation with PHA. Tblasts were then plated in suspension at a concentration of 50,000 or 75,000 cells/well in X-VIVO culture media (containing human serum albumin) and stimulated with a dilution series of recombinant IL-2 fusion proteins or human IL-2 for 72 hours at 37° C. and 5% CO₂. Activity of uncleaved and cleaved IL-2 fusion proteins was tested. Cleaved inducible IL-2 was generated by incubation with active MMP9. IL-2 activity was assessed measuring proliferation with CellTiter-Glo.

Sample fusion protein constructs are detailed in Table 3. In table 3, “L” is an abbreviation of “linker”, and “cleav. link.” is an abbreviation of “cleavable linker”. Other abbreviations “mIFNg” indicates mouse interferon gamma (IFNg); “hAlbumin” indicates human serum albumin (HSA); “mAlbumin” indicates mouse serum albumin.

TABLE 3
CONSTRUCT PERMUTATION TABLE (“6xHis” disclosed as SEQ ID NO: 354)

| Construct | |
|---|---|
| Name | Construct Description |
| ACP01 | (anti-HSA)-(cleav. link.)-mouse IFNg-(cleav. link.)-(anti-HSA)-6xHis |
| ACP02 | (anti-HSA)-(cleav. link.)-mouse IFNg-(cleav. link.)-mouse IFNg-(cleav. link.)-(anti- |
| | HSA)-6xHis |
| ACP03 | (anti-HSA)-(cleav. link.)-mouse IFNg-mouse IFNg-(cleav. link.)-(anti-HSA)-6xHis |
| ACP50 | (anti-EpCAM)-(anti-HSA)-(cleav. link.)-mouse IFNg-mouse IFNg-(cleav. link.)-(anti- |
| | HSA)-6xHis |
| ACP51 | (anti-EpCAM)-Linker-(anti-HSA)-(cleav. link.)-mIFNg-(cleav. link.)-(anti-HSA)- |
| | 6xHis |
| ACP52 | (anti-HSA)-(cleav. link.)-mIFNg-(cleav. link.)-(anti-HSA)-Linker-(anti-EpCAM)- |
| | 6xHis |
| ACP53 | mAlbumin-(cleav. link.)-mIFNg-(cleav. link.)-mAlbumin-6xHis |
| ACP54 | mAlbumin-(cleav. link.)-mIFNg-Linker-mIFNg-(cleav. link.)-mAlbumin-6xHis |
| ACP30 | (anti-HSA)-(cleav. link.)-mouse IFNg-(cleav. link.)-(anti-HSA)-(cleav. link.)-mouse |
| | IFNg-(cleav. link.)-(anti-HSA)-6xHis |
| ACP55 | (anti-HSA)-(cleav. link.)-mouse IFNg-(cleav. link.)-(anti-HSA)-(cleav. link.)-mouse |
| | IFNg-(cleav. link.)-(anti-HSA)-6xHis-C-tag |
| ACP56 | (anti-FOLR1)-Linker-(anti-HSA)-(cleav. link.)-mIFNg-(cleav. link.)-(anti-HSA)-6xHis |
| ACP57 | (anti-HSA)-(cleav. link.)-mIFNg-(cleav. link.)-(anti-HSA)-Linker-(anti-FOLR1)-6xHis |
| ACP58 | (anti-HSA)-(cleav. link.)-mIFNg-(cleav. link.)-mIFNg-(cleav. link.)-(anti-HSA)- |
| | Linker-(anti-EpCAM)-6xHis |
| ACP59 | (anti-FOLR1)-Linker-(anti-HSA)-(cleav. link.)-mIFNg-(cleav. link.)-mIFNg-(cleav. |
| | link.)-(anti-HSA)-6xHis |
| ACP60 | (anti-HSA)-(cleav. link.)-mIFNg-(cleav. link.)-mIFNg-(cleav. link.)-(anti-HSA)- |
| | Linker-(anti-FOLR1)-6xHis |
| ACP61 | (anti-HSA)-(cleav. link.)-mIFNg-(cleav. link.)-mIFNg-(cleav. link.)-(anti-HSA)- |
| | Linker-FN(CGS-2)-6xHis |
| ACP63 | anti-FN CGS-2 scFv (Vh/Vl)-6xHis |
| ACP69 | (anti-HSA)-(cleav. link.)-mouse IFNg-(cleav. link.)-(anti-HSA)-(cleav. link.)-mouse |
| | IFNg |
| ACP70 | mouse IFNg-(cleav. link.)-(anti-HSA)-(cleav. link.)-mouse IFNg-(cleav. link.)-(anti- |
| | HSA) |
| ACP71 | mouse IFNg-(cleav. link.)-mAlbumin-(cleav. link.)-mouse IFNg-(cleav. link.)- |
| | mAlbumin |
| ACP72 | mAlbumin-(cleav. link.)-mouse IFNg-(cleav. link.)-mAlbumin-(cleav. link.)-mouse |
| | IFNg |
| ACP73 | mAlbumin-(cleav. link.)-mouse IFNg-(cleav. link.)-mAlbumin-(cleav. link.)-mouse |
| | IFNg-(cleav. link.)-mAlbumin |
| ACP74 | mAlbumin-(cleav. link.)-mouse IFNg-(cleav. link.)-5mer linker-mAlbumin-5mer |
| | linker-(cleav. link.)-mouse IFNg-(cleav. link.)-mAlbumin |
| ACP75 | mAlbumin-(cleav. link.)-mouse IFNg-(cleav. link.)-10mer linker-mAlbumin-10mer |
| | linker-(cleav. link.)-mouse IFNg-(cleav. link.)-mAlbumin |
| ACP78 | (anti-HSA)-Linker-mouse_IFNg-Linker-(anti-HSA)-Linker-mouse_IFNg-Linker-(anti- |
| | HSA)_(non-cleavable_control) |
| ACP134 | Anti-HSA-(cleav. link.)-mouse_IFNg-(cleav. link.)-anti-HSA-(cleav. link.)- |
| | mouse_IFNg-(cleav. link.)-anti-HSA-L-anti-FOLR1 |
| ACP135 | Anti-FOLR1-L-HSA-(cleav. link.)-mouse_IFNg-(cleav. link.)-HSA-(cleav. link.)- |
| | mouse_IFNg-(cleav. link.)-HSA |
| ACP04 | human p40-murine p35-6xHis |
| ACP05 | human p40-human p35-6xHis |
| ACP34 | mouse p35-(cleav. link.)-mouse p40-6xHis |
| ACP35 | mouse p35-GS-(cleav. link.)-GS-mouse p40-6xHis |
| ACP36 | (anti-HSA)-(Cleav. Linker)-mouse p40-mouse p35-(Cleav. Linker)-(anti-HSA)-6xHis |
| ACP37 | (anti-EpCAM)-(anti-HSA)-(Cleav. Linker)-mouse p40-mouse p35-(Cleav. Linker)- |
| | (anti-HSA)-6xHis |
| ACP79 | (anti-EpCAM)-Linker-(anti-HSA)-(cleav. link.)-mIL12-(cleav. link.)-(Anti-HSA)- |
| | 6xHis |
| ACP80 | (anti-HSA)-(cleav. link.)-mIL12-(cleav. link.)-(anti-HSA)-Linker-(anti-EpCAM)- |
| | 6xHis |
| ACP06 | Blocker12-Linker-(cleav. link.)-human p40-Linker-mouse p35-(cleav. link.)-(anti- |
| | HSA)-6xHis |
| ACP07 | Blocker12-Linker-(cleav. link.)-human p40-Linker-mouse p35-(cleav. link.)-(anti- |
| | HSA)-Linker-(anti-FOLR1)-6xHis |
| ACP08 | (anti-FOLR1)-Linker-Blocker12-Linker-(cleav. link.)-human p40-Linker-mouse p35- |
| | (cleav. link.)-(anti-HSA)-6xHis |
| ACP09 | (anti-HSA)-Linker-Blocker12-Linker-(cleav. link.)-human p40-Linker-mouse p35- |
| | 6xHis |
| ACP10 | (anti-HSA)-(cleav. link.)-human p40-L-mouse p35-(cleav. link.)-Linker-Blocker12-6xHis |
| ACP11 | Human_p40-Linker-mouse_p35-(cleav. link.)-Linker-Blocker12-Linker-(anti-HSA)-6xHis |
| ACP91 | human_p40-Linker-mouse_p35-Linker-Linker-Blocker-Linker-(anti-HSA)_(non- |
| | cleavable control) |
| ACP136 | human p40-L-mouse p35-(cleav. link.)-Blocker |
| ACP138 | human_p40-L-mouse_p35-(cleav. link.)-Blocker-L-(anti-HSA)-L-FOLR1 |
| ACP139 | Anti-FOLR1-L-human_p40-L-mouse_p35-(cleav. link.)-Blocker12-L-(anti-HSA) |
| ACP140 | Anti-FOLR1-(cleav. link.)-human_p40-L-mouse_p35-(cleav. link.)-Blocker12-L-(anti-HSA) |
| ACP12 | (anti-EpCAM)-IL2-(cleav. link.)-(anti-HSA)-blocker2-6xHis |
| ACP13 | (anti-EpCAM)-Blocker2-(anti-HSA)-(cleav. link.)-IL2-6xHis |
| ACP14 | Blocker2-Linker-(cleav. link.)-IL2- (cleav. link.)-(anti-HSA)-6xHis |
| ACP15 | Blocker2-Linker-(anti-HSA)-Linker-(cleav. link.)- IL2 -6xHis |
| ACP16 | IL2-(cleav. link.)-(anti-HSA)-Linker-(cleav. link.)-Blocker2-6xHis |
| ACP17 | (anti-EpCAM)-Linker-IL2-(cleav. link.)-(anti-HSA)-Linker-(cleav. link.)-Blocker2-6xHis |
| ACP18 | (anti-EpCAM)-Linker-IL2-(clcav. link.)-(anti-HSA)-Linker-vh(cleav. link.)vl-6xHis |
| ACP19 | IL2-(cleav. link.)-Linker-Blocker2-Linker-(anti-HSA)-Linker-(anti-EpCAM) -6xHis |
| ACP20 | IL2-(cleav. link.)-Blocker2-6xHis |
| ACP21 | IL2-(cleav. link.)-Linker-Blocker2-6xHis |
| ACP22 | IL2-(cleav. link.)-Linker-blocker-(cleav. link.)-(anti-HSA)-Linker-(anti-EpCAM)-6xHis |
| ACP23 | (anti-FOLR1)-(cleav. link.)-Blocker2-Linker-(cleav. link.)-(anti-HSA)-(cleav. link.)-IL2-6xHis |
| ACP24 | (Blocker2)-(cleav. link.)-(IL2)-6xHis |
| ACP25 | Blocker2-Linker-(cleav. link.)-IL2-6xHis |
| ACP26 | (anti-EpCAM)-Linker-IL2-(cleav. link.)-(anti-HSA)-Linker-blocker(NARA1 Vh/Vl) |
| ACP27 | (anti-EpCAM)-Linker-IL2-(cleav. link.)-(anti-HSA)-Linker-blocker(NARA1 Vl/Vh) |
| ACP28 | IL2-(cleav. link.)-Linker-Blocker2-(NARA1 Vh/Vl)-Linker-(anti-HSA)-Linker-(anti-EpCAM) |
| ACP29 | IL2-(cleav. link.)-Linker-Blocker2-(NARA1 Vl/Vh)-Linker-(anti-HSA)-Linker-(anti-EpCAM) |
| ACP38 | IL2-(cleav. link.)-blocker-(anti-HSA)-(anti-EpCAM)-6xHis |
| ACP39 | (anti-EpCAM)-(cleav. link.)-(anti-HSA)-(cleav. link.)-Blocker2-(cleav. link.)-IL-2-6xHis |
| ACP40 | CD25ecd-Linker-(cleav. link.)-IL2-6xHis |
| ACP41 | IL2-(cleav. link.)-Linker-CD25ecd-6xHis |
| ACP42 | (anti-HSA)-Linker-CD25ecd-Linker-(cleav. link.)-IL2-6xHis |
| ACP43 | IL2-(cleav. link.)-Linker-CD25ecd-Linker-(anti-HSA)-6xHis |
| ACP44 | IL2-(cleav. link.)-Linker-CD25ecd-(cleav. link.)-(anti-HSA)-6xHis |
| ACP45 | (anti-HSA)-(cleav. link.)-Blocker2-Linker-(cleav. link.)-IL2-6xHis |
| ACP46 | IL2-(cleav. link.)-linkerL-vh(cleav. link.)vl-Linker-(anti-HSA)-L-(anti-EpCAM)-6xHis |
| ACP47 | (anti-EpCAM)-Linker-IL2-(Cleavable Linker)-(anti-HSA)-Linker-Blocker2-6xHis |
| ACP48 | IL2-(cleav. link.)-Blocker2-Linker-(anti-HSA)-6xHis |
| ACP49 | IL2-(cleav. link.)-Linker-Blocker2-Linker-(anti-HSA)-6xHis |
| ACP92 | (anti-HSA)-(16mer Cleav. Link.)-IL2-(16mer Cleav. Link.)-(anti-HSA)-6XHis |
| ACP93 | (anti-EpCAM)-(anti-HSA)-(anti-EpCAM)-Blocker2-(cleav. link.)-IL2-6xHis |
| ACP94 | (anti-EpCAM)-(anti-HSA)-Blocker2-(cleav. link.)-IL2-6xHis |
| ACP95 | (anti-EpCAM)-(anti-HSA)-(cleav. link.)-IL2-6xHis |
| ACP96 | (anti-EpCAM)-(16mer cleav. link.)-IL2-(16mer cleav. link.)-(anti-HSA) |
| ACP97 | (anti-EpCAM)-(anti-HSA)-(cleav. link.)-IL2-(cleav. link.)-(anti-HSA)-6xHis |
| ACP99 | (anti-EpCAM)-Linker-IL2-(cleav. link.)-(anti-HSA)-6xHis |
| ACP100 | (anti-EpCAM)-Linker-IL2-6xHis |
| ACP101 | IL2-(cleav. link.)-(anti-HSA)-6xHis |
| ACP102 | (anti-EpCAM)-(cleav. link.)-IL2-(cleav. link.)-(anti-HSA)-Linker-blocker-6xHis |
| ACP103 | IL2-(cleav. link.)-Linker-Blocker2-Linker-(anti-HSA)-Linker-(antiI-FOLR1)-6xHis |
| ACP104 | (anti-FOLR1)-IL2-(cleav. link.)-(anti-HSA)-Linker-Blocker2-6xHis |
| ACP105 | Blocker2-Linker-(cleav. link.)-IL2-(cleav. link.)-(anti-HSA)-Linker-(anti-FOLR1)-6xHis |
| ACP106 | (anti-FOLR1)-Linker-(anti-HSA)-(cleav. link.)-blocker-Linker-(cleav. link.)-IL2 -6xHis |
| ACP107 | Blocker2-Linker-(anti-HSA)-(cleav. link.)-IL2-Linker-(anti-FOLR1)-6xHis |
| ACP108 | (anti-EpCAM)-IL2-(Dually cleav. link.)-(anti-HSA)-Linker-blocker-6xHis |
| ACP117 | anti-FN CGS-2 scFv (Vh/Vl)-6xHis |
| ACP118 | NARA1 Vh/Vl non-cleavable |
| ACP119 | NARA1 Vh/Vl cleavable |
| ACP120 | NARA1 Vl/Vh non-cleavable |
| ACP121 | NARA1 Vl/Vh cleavable |
| ACP124 | IL2-Linker-(anti-HSA)-Linker-Linker-blocker_(non-cleavable_control) |
| ACP132 | IL2-L-HSA |
| ACP141 | IL2-L-human_Albumin |
| ACP142 | IL2-(cleav. link.)-human_Albumin |
| ACP144 | IL2-(cleav. link.)-HSA-(cleav.-link.)blocker-L-(anti-FOLR1) |
| ACP145 | Anti-FOLR1-L-IL2-(cleav. link.)-HSA-Linker-(cleav. link.)-blocker2 |
| ACP146 | Anti-FOLR1-(cleav. link)-IL2-(cleav. link.)-HSA-Linker-(cleav. link.)-blocker2 |
| ACP133 | IL2-6x His |
| ACP147 | IL2-(cleav. Linker)-(anti-HSA)-Linker-(cleav. link.)-blocker2-L-(anti-EpCAM) |
| ACP148 | (anti-EpCAM)-L-IL2-(cleav. link.)-(anti-HSA)-L-(cleav. Linker)-blocker2 |
| ACP149 | (anti-EpCAM)-(cleav. link.)-IL2-(cleav. Linker)-(anti-HSA)-L-(cleav. Linker)-blocker2 |
| ACP31 | (anti-HSA)-(cleav. link.)-mIFNa1-(cleav. link.)-(anti-HSA) |
| ACP32 | (anti-HSA)-(cleav. link.)-mIFNa1(N + C trunc)-(cleav. link.)-(anti-HSA) |
| ACP33 | (anti-HSA)-(cleav. link.)-mIFNa1(C trunc)-(cleav. link.)-(anti-HSA) |
| ACP131 | mIFNa1 |
| ACP125 | Anti-HSA-(cleav. link.)-mIFNa1 |
| ACP126 | mIFNa1-(cleav. link.)-(anti-HSA) |
| ACP127 | Mouse_Albumin-(cleav. Link.)-mIFNa1-(cleav link)-mouse_Albumin |
| ACP128 | Mouse_Albumin-(cleav. link.)-mIFNa1 |
| ACP129 | mIFNa1-(cleav. link.)-mAlb |
| ACP150 | (Anti-FOLR1)-L-(anti-HSA)-(cleav. Link.)-mIFNa1-(cleav. Link.)-(anti-HSA) |
| ACP151 | Anti-FOLR1-L-(anti-HSA)-(cleav. Link.)-mIFNa1-(cleav. Link.)-(anti-HSA)-L-(anti-FLOR1) |
| ACP152 | (anti-HSA)-L-mIFNa1-L-(anti-HSA)_(non-cleavable_control) |
| ACP153 | IL2-(cleav. link.)-(anti-HSA)-linker(cleav. link.)-blocker2 |
| ACP154 | IL2-(cleav. link.)-(anti-HSA)-linker(cleav. link.)-blocker2 |
| ACP155 | IL2-(cleav. link.)-(anti-HSA)-linker(cleav. link.)-blocker2 |
| ACP156 | IL2-(cleav. link.)-(anti-HSA)-linker(cleav. link.)-blocker2 |
| ACP157 | IL2-(cleav. link.)-(anti-HSA)-linker(cleav. link.)-blocker2 |
| ACP200 | mAlb(D3)-X-mouse-IFNa-X-mAlb(D3)_(X = MMP9-M) |
| ACP201 | mAlb(D1-L-D3)-X-mouse-IFNa-X-mAlb(D1-L-D3)_(X = MMP9-M) |
| ACP202 | HSA-X-mIFNa1-X-HSA_(X = MMP9-M + 17aa) |
| ACP203 | HSA-X-mIFNa1-X-HSA_(X = MMP14-1) |
| ACP204 | HSA-X-mIFNa1-X-HSA_(X = CTSL1-1) |
| ACP205 | HSA-X-mIFNa1-X-HSA_(X = ADAM17-2) |
| ACP206 | HSA-X-Human_IFNA2b-X-HSA_(X = MMP14-1) |
| ACP207 | HSA-X-Human_IFNA2b-X-HSA_(X = CTSL1-1) |
| ACP208 | HSA-X-Human_IFNA2b-X-HSA_(X = ADAM17-2) |
| ACP211 | HSA-X-mouse-IFNg-X-IFNa-X-mouse-IFNg-X-HSA_(X = MMP9-M) |
| ACP213 | mAlb(D3)-X-mouse-IFNg-X-mAlb(D3)-X-mouse-IFNg-X-mAlb(D3)_(X = MMP9-M) |
| ACP214 | mAlb(D1-L-D3)-X-mouse-IFNg-X-mAlb(D1-L-D3)-X-mouse-IFNg-X-mAlb(D1-L-D3)_(X = MMP9-M) |
| ACP215 | HSA-X-mouse-IFNg-X-HSA-X-mouse-IFNg-X-HSA_(X = MMP9-M + 17aa) |
| ACP240 | HSA-L-human_p40-L-mouse_p35-LL-Blocker_(non-cleavable; |
| | Blocker = briakinumab_Vl/Vh) |
| ACP241 | mAlb-X-human_p40-L-mouse_p35-XL-Blocker_(X = MMP9-M; |
| | Blocker = briakinumab_Vl/Vh) |
| ACP242 | human_p40-L-mouse_p35-XL-Blocker-X-mAlb_(X = MMP9-M; |
| | Blocker = briakinumab_Vl/Vh) |
| ACP243 | mIgG1_Fc-X-human_p40-L-mouse_p35-XL-Blocker_(X = MMP9-M; |
| | Blocker = briakinumab_Vl/Vh) |
| ACP244 | human_p40-L-mouse_p35-XL-Blocker-X-mIgGl_Fc_(X = MMP9-M; |
| | Blocker = briakinumab_Vl/Vh) |
| ACP245 | HSA-X-human_p40-L-mouse_p35-XL-Blocker(cleavable)_(X = MMP9-M; |
| | Blocker = briakinumab_Vl-X-Vh) |
| ACP247 | HSA-X-human_p40-L-mouse_p35-XL-Blocker_(Blocker = 3CYT5; |
| | X = MMP9-M) |
| ACP284 | HSA-X-mouse_p35-XL-Blocker_(Blocker = briakinumab_Vl/Vh; |
| | X = MMP9-M) |
| ACP285 | HSA-X-human_p40_C199S-L-mouse_p35 C92S-XL-Blocker_(Blocker = |
| | briakinumab_Vl/Vh; X = MMP9-M) |
| ACP286 | HSA-X-human p40-L(4xG4S (SEQ ID NO: 453))-mouse p35-XL-Blocker_(Blocker = |
| | briakinumab_Vl/Vh; X = MMP9-M) |
| ACP287 | HSA-X-human_p40_mouse_p35-XL-Blocker_(Blocker = |
| | briakinumab_Vl/Vh_VH44-VL100_disulfide; X = MMP9-M) |
| ACP288 | HSA-X-human_p40_mouse_p35-XL-Blocker_(Blocker = |
| | briakinumab_Vl/Vh_VH105-VL43_disulfide; X = MMP9-M) |
| ACP289 | Geneart_WW0048_IL2-X-HSA-LX-blocker_Fusion_protein-6xHis |
| ACP290 | IL2-X-HSA-LX-blocker_(X = MMP9-M; Blocker = 3TOW69) |
| ACP291 | IL2-X-HSA-LX-blocker_(X = MMP9-M; Blocker = 3TOW85) |
| ACP292 | IL2-X-HSA-LX-blocker_(X = MMP9-M; Blocker = 2TOW91) |
| ACP296 | IL2-X-HSA-LX-blocker(cleavable)_(X = MMP9-M; Blocker = MT204_Vh-X-Vl) |
| ACP297 | IL2-X-HSA-LX-blocker(A46L)_(X = MMP9-M; Blocker = MT204_Vh/Vl) |
| ACP298 | IL2-X-HSA-LX-blocker(A46G)_(X = MMP9-M; Blocker = MT204_Vh/Vl) |
| ACP299 | IL2(Cysl45Ser)-X-HSA-LX-blocker_(X = MMP9-M; Blocker = MT204_Vh/Vl) |
| ACP300 | IL2-X-hAlb-LX-blocker_(X = MMP9-M; Blocker = MT204_Vh/Vl) |
| ACP302 | IL2-X-mAlb-LX-blocker_(X = MMP9-M; Blocker = MT204_Vh/Vl) |
| ACP303 | mAlb-X-IL2(Nterm-41)-X-mALB_(X = MMP9-M) |
| ACP304 | IL2-X-HSA-LX-blocker-XL-CD25ecd_(X = MMP9-M; Blocker = MT204_Vh/Vl) |
| ACP305 | CD25ecd-LX-IL2-X-HSA-LX-blocker_(X = MMP9-M; Blocker = MT204_Vh/Vl) |
| ACP306 | IL2-XL-CD25ecd-X-HSA-LX-blocker_(X = MMP9-M; Blocker = MT204_Vh/Vl) |
| ACP309 | IL2-X-HSA-LX-blocker(A46S)_(X = MMP9-M; Blocker = MT204_Vh/Vl) |
| ACP310 | IL2-X-HSA-LX-blocker(QAPRL_FR2)_(X = MMP9-M; Blocker = MT204_Vh/Vl) |
| ACP311 | IL2-X-IgG4_Fc(S228P)-LX-Blocker_(X = MMP9-M; Blocker = MT204_Vh/Vl) |
| ACP312 | IgG4_Fc(S228P)-X-IL2-LX-Blocker_(X = MMP9-M; Blocker = MT204_Vh/Vl) |
| ACP313 | IL2-XL-Blocker-X-IgG4_Fc(S228P)_(X = MMP9-M; Blocker = MT204_Vh/Vl) |
| ACP314 | mIgG1_Fc-X-IL2-LX-Blocker_(X = MMP9-M; Blocker = MT204_Vh/Vl) |
| ACP336 | IL2-X-anti-HSA-LX-blocker_(Blocker = |
| | VHVL.F2.high.A02_Vh-X-Vl_A46S; X = MMP14-1) |
| ACP337 | IL2-X-anti-HSA-LX-blocker_(Blocker = |
| | VHVL.F2.high.A02_Vh/Vl_A46S; X = MMP14-1) |
| ACP338 | IL2-X-anti-HSA-LX-blocker_(Blocker = VHVL.F2.high.F03_Vh-X-Vl; X = MMP14-1) |
| ACP339 | IL2-X-anti-HSA-LX-blocker_(Blocker = VHVL.F2.high.F03_Vh/Vl; X = MMP14-1) |
| ACP340 | IL2-X-anti-HSA-LX-blocker_(Blocker = Hu2TOW91_B; X = MMP14-1) |
| ACP341 | IL2-X-anti-HSA-LX-blocker_(Blocker = Hu3TOW85_A; X = MMP14-1) |
| ACP342 | CD25ecd_C213S-LX-IL2-X-anti-HSA-LX-blocker_(Blocker = |
| | VHVL.F2.high.A02_Vh-X-Vl_A46S; X = MMP14-1) |
| ACP343 | CD25ecd_C213S-LX-IL2-X-anti-HSA-LX-blocker_(Blocker = |
| | VHVL.F2.high.A02_Vh/Vl_A46S; X = MMP14-1) |
| ACP344 | CD25ecd_C213S-LX-IL2-X-anti-HSA-LX-blocker_(Blocker = |
| | VHVL.F2.high.F03_Vh-X-Vl; X = MMP14-1) |
| ACP345 | CD25ecd_C213S-LX-IL2-X-anti-HSA-LX-blocker_(Blocker = |
| | VHVL.F2.high.F03_Vh/Vl; X = MMP14-1) |
| ACP346 | CD25ecd_C213S-LX-IL2-X-anti-HSA-LX-blocker_(Blocker = |
| | Hu2TOW91_B; X = MMP14-1) |
| ACP347 | CD25ecd_C213S-LX-IL2-X-anti-HSA-LX-blocker_(Blocker = |
| | Hu3TOW85_A; X = MMP14-1) |
| ACP348 | IgG4_Fc(S228P)-X-IL2-LX-Blocker_(Blocker = |
| | VHVL.F2.high.A02_Vh-X-Vl_A46S; X = MMP14-1) |
| ACP349 | IgG4_Fc(S228P)-X-IL2-LX-Blocker_(Blocker = |
| | VHVL.F2.high.A02_Vh\Vl_A46S; X = MMP14-1) |
| ACP350 | IgG4_Fc(S228P)-X-IL2-LX-Blocker_(Blocker = |
| | VHVL.F2.high.F03_Vh-X-Vl; X = MMP14-1) |
| ACP351 | IgG4_Fc(S228P)-X-IL2-LX-Blocker (Blocker = |
| | VHVL.F2.high.F03_Vh\Vl; X = MMP14-1) |
| ACP352 | IgG4_Fc(S228P)-X-IL2-LX-Blocker_(Blocker = Hu2TOW91_B; X = MMP14-1) |
| ACP353 | IgG4_Fc(S228P)-X-IL2-LX-Blocker_(Blocker = Hu3TOW85_A; X = MMP14-1) |
| ACP354 | IgG4_Fc(S228P)-X-CD25ecd_C213S-LX-IL2-LX-Blocker_(Blocker = |
| | VHVL.F2.high.A02_Vh-X-Vl_A46S; X = MMP14-1) |
| ACP355 | IgG4_Fc(S228P)-X-CD25ecd_C213S-LX-IL2-LX-Blocker_(Blocker = |
| | VHVL.F2.high.A02_Vh\Vl_A46S; X = MMP14-1) |
| ACP356 | IgG4_Fc(S228P)-X-CD25ecd_C213S-LX-IL2-LX-Blocker_(Blocker = |
| | VHVL.F2.high.F03_Vh-X-V1; X = MMP14-1) |
| ACP357 | IgG4_Fc(S228P)-X-CD25ecd_C213S-LX-IL2-LX-Blocker_(Blocker = |
| | VHVL.F2.high.F03_Vh\Vl; X = MMP14-1) |
| ACP358 | IgG4_Fc(S228P)-X-CD25ecd_C213S-LX-IL2-LX-Blocker_(Blocker = |
| | Hu2TOW91_B; X = MMP14-1) |
| ACP359 | IgG4_Fc(S228P)-X-CD25ecd_C213S-LX-IL2-LX-Blocker_(Blocker = |
| | Hu3TOW85_A; X = MMP14-1) |
| ACP371 | IL2-X-anti-HSA-LX-blocker_(Blocker = |
| | MT204_Vh/Vl_VH44-VL100_disulfide; X = MMP14-1) |
| ACP372 | IL2-X-anti-HSA-LX-blocker_(Blocker = |
| | MT204_Vh/Vl_VH105-VL43_disulfide; X = MMP14-1) |
| ACP373 | IL2-X-anti-HSA-LX-blocker_(Blocker = |
| | VHVL.F2.high.A02_Vh/Vl_VH44-VL100_disulfide; X = MMP14-1) |
| ACP374 | IL2-X-anti-HSA-LX-blocker_(Blocker = |
| | VHVL.F2.high.A02_Vh/Vl_VH105-VL43_disulfide; X = MMP14-1) |
| ACP375 | IL2-X-anti-HSA-LX-blocker_(Blocker = |
| | VHVL.F2.high.F03_Vh/Vl_VH44-VL100_disulfide; X = MMP14-1) |
| ACP376 | IL2-X-anti-HSA-LX-blocker_(Blocker = |
| | VHVL.F2.high.F03_Vh/Vl_VH105-VL43_disulfideX = MMP14-1) |
| ACP377 | IL2-X-anti-HSA-LX-blocker_(Blocker = Hu2TOW91_A; X = MMP14-1) |
| ACP378 | IL2-X-anti-HSA-LX-Heavy_blocker_Fab_(Blocker = MT204_VH-CH1; X = MMP14-1) |
| ACP379 | IgG4_Fc(S228P)-X-IL2-LX-Heavy_blocker_Fab_(Blocker = |
| | MT204_VH-CH1; X = MMP14-1) |
| ACP383 | IgG4_Fc(S228P)-X-IL2-LX-blocker_(Blocker = |
| | MT204_Vh/Vl_VH44-VL100_disulfide; X = MMP14-1) |
| ACP384 | IgG4_Fc(S228P)-X-IL2-LX-blocker_(Blocker = |
| | MT204_Vh/Vl_VH105-VL43_disulfide; X = MMP14-1) |
| ACP385 | IgG4_Fc(S228P)-X-IL2-LX-blocker_(Blocker = VHVL.F2.high.A02_Vh/Vl_VH44-VL100_disulfide; X = MMP14-1) |
| ACP386 | IgG4_Fc(S228P)-X-IL2-LX-blocker_(Blocker = VHVL.F2.high.A02_Vh/Vl_VH105-VL43_disulfide; X = MMP14-1) |
| ACP387 | IgG4_Fc(S228P)-X-IL2-LX-blocker_(Blocker = VHVL.F2.high.F03_Vh/Vl_VH44-VL100_disulfide; X = MMP14-1) |
| ACP388 | IgG4_Fc(S228P)-X-IL2-LX-blocker_(Blocker = VHVL.F2.high.F03_Vh/Vl_VH105-VL43_disulfide; X = MMP14-1) |
| ACP389 | IgG4_Fc(S228P)-X-IL2-LX-blocker_(Blocker = Hu2TOW91_A; X = MMP14-1) |
| ACP390 | IL2-X-anti-HSA-LX-blocker_(Blocker = VHVL.F2.high.A02_Vh/Vl_A46S_VH44-VL100_disulfide; X = MMP14-1) |
| ACP391 | IgG4_Fc(S228P)-X-IL2-LX-blocker_(Blocker = |
| | VHVL.F2.high.A02_Vh/Vl_A46S_VH44-VL100_disulfide; X = MMP14-1) |
| ACP392 | IL2-XL-CD25ecd_C213S-X-HSA-LX-blocker_(Blocker = |
| | VHVL.F2.high.A02_Vh_G44C_Vl_A46S_G100C; X = MMP14-1) |
| ACP393 | IL2-XL-CD25ecd_C213S-X-HSA-LX-blocker_(Blocker = |
| | VHVL.F2.high.A02_Vh_Q105C_Vl_A43C; X = MMP14-1) |
| ACP394 | IL2-XL-CD25ecd_C213S-X-HSA-LX-blocker_(Blocker = |
| | VHVL.F2.high.F03_Vh_G44C_Vl_G100C; X = MMP14-1) |
| ACP395 | IL2-XL-CD25ecd_C213S-X-HSA-LX-blocker_(Blocker = |
| | VHVL.F2.high.F03_Vh_Q105C_Vl_A43C; X = MMP14-1) |
| ACP396 | IL2-XL-CD25ecd_C213S-X-HSA-LX-blocker_(Blocker = |
| | Hu2TOW91_A; X = MMP14-1) |
| ACP397 | IL2-XL-CD25ecd_C213S-X-HSA-LX-blocker_(Blocker = |
| | Hu2TOW91_B; X = MMP14-1) |
| ACP398 | IL2-XL-CD25ecd_C213S-X-HSA-LX-Heavy_blocker_Fab_(Blocker = |
| | MT204_VH-CH1; X = MMP14-1) |
| ACP399 | Blocker-XL-HSA-X-IL2(Nterm-41)-X-HSA)_(Blocker = |
| | VHVL.F2.high.A02_Vh_G44C_Vl_A46S_G100C; X = MMP14-1) |
| ACP400 | Blocker-XL-HSA-X-IL2(Nterm-41)-X-HSA_(Blocker = |
| | VHVL.F2.high.A02_Vh_Q105C_Vl_A43C; X = MMP14-1) |
| ACP401 | Blocker-XL-HSA-X-IL2(Nterm-41)-X-HSA_(Blocker = |
| | VHVL.F2.high.F03_Vh_G44C_Vl_G100C; X = MMP14-1) |
| ACP402 | Blocker-XL-HSA-X-IL2(Nterm-41)-X-HSA_(Blocker = |
| | VHVL.F2.high.F03_Vh_Q105C_Vl_A43C; X = MMP14-1) |
| ACP403 | Blocker-XL-HSA-X-IL2(Nterm-41)-X-HSA_(Blocker = Hu2TOW91_A; X = MMP14-1) |
| ACP404 | Blocker-XL-HSA-X-IL2(Nterm-41)-X-HSA_(Blocker = Hu2TOW91_B; X = MMP14-1) |
| ACP405 | Heavy_Blocker_Fab-XL-HSA-X-IL2(Nterm-41)-X-HSA_(Blocker = |
| | MT204_VH-CH1; X = MMP14-1) |
| ACP406 | mIgG1_Fc(S228P)-X-IL2-LX-Heavy_blocker_Fab_(Blocker = |
| | MT204_VH-CH1; X = MMP14-1) |
| ACP407 | mIgG1_Fc(S228P)-X-IL2-LX-blocker_(Blocker = |
| | VHVL.F2.high.A02_Vh/Vl_VH44-VL100_disulfide; X = MMP14-1) |
| ACP408 | mIgG1_Fc(S228P)-X-IL2-LX-blocker_(Blocker = |
| | VHVL.F2.high.A02_Vh/Vl_A46S_VH44-VL100_disulfide; X = MMP14-1) |
| ACP409 | mIgG1_Fc(S228P)-X-IL2-LX-blocker_(Blocker = |
| | VHVL.F2.high.A02_Vh/Vl_VH105-VL43_disulfidel; X = MMP14-1) |
| ACP410 | mIgG1_Fc(S228P)-X-IL2-LX-blocker_(Blocker = |
| | VHVL.F2.high.F03_Vh/Vl_VH44-VL100_disulfidel; X = MMP14-1) |
| ACP411 | mIgG1_Fc(S228P)-X-IL2-LX-blocker_(Blocker = |
| | VHVL.F2.high.F03_Vh/Vl_VH105-VL43_disulfidel; X = MMP14-1) |
| ACP412 | mIgG1_Fc(S228P)-X-IL2-LX-blocker_(Blocker = Hu2TOW91_A; X = MMP14-1) |
| ACP413 | CD25_213S-L-Kappa_blocker_Fab_(Blocker = VHVL.F2.high.A02_A46S_Kappa) |
| ACP414 | CD25_213S-L-Kappa_blocker_Fab_(Blocker = VHVL.F2.high.F03_Kappa) |
| ACP415 | IL2-XL-blocker-L-CD25_213S-X-HSA_Blocker = |
| | VHVL.F2.high.A02_Vh_G44C_Vl_A46S_G100C; X = MMP14-1) |
| ACP416 | IL2-XL-blocker-L-CD25_213S-X-HSA_(Blocker = |
| | VHVL.F2.high.A02_Vh_Q105C_Vl_A43C; X = MMP14-1) |
| ACP417 | IL2-XL-blocker-L-CD25_213S-X-HSA_(Blocker = |
| | VHVL.F2.high.F03_Vh_G44C_Vl_G100C; X = MMP14-1) |
| ACP418 | IL2-XL-blocker-L-CD25_213S-X-HSA_(Blocker = |
| | VHVL.F2.high.F03_Vh_Q105C_Vl_A43C; X = MMP14-1) |
| ACP419 | IL2-XL-blocker-L-CD25_213S-X-HSA_(Blocker = Hu2TOW91_A; X = MMP14-1) |
| ACP420 | IL2-XL-blocker-L-CD25_213S-X-HSA_(Blocker = Hu2TOW91_B; X = MMP14-1) |
| ACP421 | HSA-X-blocker-L-CD25_213S-LX-IL2_(Blocker = |
| | VHVL.F2.high.A02_Vh_G44C_Vl_A46S_G100C; X = MMP14-1) |
| ACP422 | HSA-X-blocker-L-CD25_213S-LX-IL2_(Blocker = |
| | VHVL.F2.high.A02_Vh_Q105C_Vl_A43C; X = MMP14-1) |
| ACP423 | HSA-X-blocker-L-CD25_213S-LX-IL2_(Blocker = |
| | VHVL.F2.high.F03_Vh_G44C_Vl_G100C; X = MMP14-1) |
| ACP424 | HSA-X-blocker-L-CD25_213S-LX-IL2_(Blocker = |
| | VHVL.F2.high.F03_Vh_Q105C_Vl_A43C; X = MMP14-1) |
| ACP425 | HSA-X-blocker-L-CD25_213S-LX-IL2_(Blocker = Hu2TOW91_A; X = MMP14-1) |
| ACP426 | HSA-X-blocker-L-CD25_213S-LX-IL2_(Blocker = Hu2TOW91_B; X = MMP14-1) |
| ACP427 | IL2-X-anti-HSA-LX-Blocker1-L-Blocker2_(Blocker1 = |
| | VHVL.F2.high.A02_Vh_G44C_Vl_A46S_G100C, |
| | Blocker2 = Hu2TOW91_A; X = MMP14-1) |
| ACP428 | IL2-X-anti-HSA-LX-Blocker1-L-Blocker2_(Blocker1 = |
| | VHVL.F2.high.A02_Vh_Q105C_Vl_A43C, |
| | Blocker2 = Hu2TOW91_A; X = MMP14-1) |
| ACP429 | IL2-X-anti-HSA-LX-Blocker1-L-Blocker2_(Blocker1 = |
| | VHVL.F2.high.F03_Vh_G44C_Vl_G100C, |
| | Blocker2 = Hu2TOW91_A; X = MMP14-1) |
| ACP430 | IL2-X-anti-HSA-LX-Blocker1-L-Blocker2_(Blocker1 = |
| | VHVL.F2.high.F03_Vh_Q105C_Vl_A43C, |
| | Blocker2 = Hu2TOW91_A; X = MMP14-1) |
| ACP431 | IL2-X-anti-HSA-LX-Blocker1-L-Blocker2_(Blocker1 = |
| | VHVL.F2.high.A02_Vh_G44C_Vl_A46S_G100C, |
| | Blocker2 = Hu2TOW91_B; X = MMP14-1) |
| ACP432 | IL2-X-anti-HSA-LX-Blocker1-L-Blocker2_(Blocker1 = |
| | VHVL.F2.high.A02_Vh_Q105C_Vl_A43C, |
| | Blocker2 = Hu2TOW91_B; X = MMP14-1) |
| ACP433 | IL2-X-anti-HSA-LX-Blocker1-L-Blocker2_(Blocker1 = |
| | VHVL.F2.high.F03_Vh_G44C_Vl_G100C, |
| | Blocker2 = Hu2TOW91_B; X = MMP14-1) |
| ACP434 | IL2-X-anti-HSA-LX-Blocker1-L-Blocker2_(Blocker1 = |
| | VHVL.F2.high.F03_Vh_Q105C_Vl_A43C, |
| | Blocker2 = Hu2TOW91_B; X = MMP14-1) |
| ACP439 | IL2-X-anti-HSA-LX-blocker_(Blocker = |
| | VHVL.F2.high.C07_Vh/Vl; X = MMP14-1) |
| ACP440 | IL2-X-anti-HSA-LX-blocker_(Blocker = |
| | VHVL.F2.high.C07_Vh/Vl_A46S; X = MMP14-1) |
| ACP441 | IL2-X-anti-HSA-LX-blocker_(Blocker = |
| | VHVL.F2.high.C07_Vh/Vl_A46L; X = MMP14-1) |
| ACP442 | IL2-X-anti-HSA-LX-blocker_(Blocker = |
| | VHVL.F2.high.C07_Vh/Vl_A46S_VH44-VL100_disulfide; X = MMP14-1) |
| ACP443 | IL2-X-anti-HSA-LX-blocker_(Blocker = |
| | VHVL.F2.high.C07_Vh/Vl_A46L_VH44-VL100_disulfide; X = MMP14-1) |
| ACP444 | IL2-X-anti-HSA-LX-blocker_(Blocker = |
| | VHVL.F2.high.C07_Vh/Vl_VH105-VL43_disulfide; X = MMP14-1) |
| ACP445 | IL2-X-anti-HSA-LX-blocker_(Blocker = VHVL.F2.high.A02_Vh-X-Vl_A46L; |
| | X = MMP14-1) |
| ACP446 | IL2-X-anti-HSA-LX-blocker_(Blocker = VHVL.F2.high.A02_Vh/Vl_A46L; X = MMP14-1) |
| ACP447 | IL2-X-anti-HSA-LX-blocker_(Blocker = |
| | VHVL.F2.high.A02_Vh/Vl_A46L_VH44-VL100_disulfide; X = MMP14-1) |
| ACP451 | IL2-X-anti-HSA-LX-blocker_(Blocker = VHVL.F2.high.A02_Vh/Vl_A46S; X = CTSL1-1) |
| ACP452 | IL2-X-anti-HSA-LX-blocker_(Blocker = VHVL.F2.high.F03_Vh/Vl; X = CTSL1-1) |
| ACP453 | IL2-X-anti-HSA-LX-blocker_(Blocker = |
| | VHVL.F2.high.A02_Vh/Vl_A46S_VH44-VL100_disulfide; X = CTSL1-1) |
| ACP454 | IL2-X-anti-HSA-LX-blocker_(Blocker = |
| | VHVL.F2.high.A02_Vh/Vl_VH105-VL43_disulfidel; X = CTSL1-1) |
| ACP455 | IL2-X-anti-HSA-LX-blocker_(Blocker = |
| | VHVL.F2.high.F03_Vh/Vl_VH44-VL100_disulfide; X = CTSL1-1) |
| ACP456 | IL2-X-anti-HSA-LX-blocker_(Blocker = |
| | VHVL.F2.high.F03_Vh/Vl_VH105-VL43_disulfideX = CTSL 1-1) |
| ACP457 | IL2-X-anti-HSA-LX-Heavy_blocker_Fab_(Blocker = MT204_VH-CH1; X = CTSL1-1) |
| ACP458 | IgG4_Fc(S228P)-X-IL2-LX-Heavy_blocker_Fab_(Blocker = |
| | MT204_VH-CH1; X = CTSL1-1) |
| ACP459 | IgG4_Fc(S228P)-X-IL2-LX-Blocker_(Blocker = |
| | VHVL.F2.high.A02_Vh\Vl_A46S; X = CTSL1-1) |
| ACP460 | IgG4_Fc(S228P)-X-IL2-LX-Blocker_(Blocker = |
| | VHVL.F2.high.F03_Vh\Vl; X = CTSL1-1) |
| ACP461 | IgG4_Fc(S228P)-X-IL2-LX-blocker_(Blocker = |
| | VHVL.F2.high.A02_Vh/Vl_A46S_VH44-VL100_disulfide; X = CTSL1-1) |
| ACP462 | IgG4_Fc(S228P)-X-IL2-LX-blocker_(Blocker = |
| | VHVL.F2.high.A02_Vh/Vl_VH105-VL43_disulfidel; X = CTSL1-1) |
| ACP463 | IgG4_Fc(S228P)-X-IL2-LX-blocker_(Blocker = |
| | VHVL.F2.high.F03_Vh/Vl_VH44-VL100_disulfidel; X = CTSL1-1) |
| ACP464 | IgG4_Fc(S228P)-X-IL2-LX-blocker_(Blocker = |
| | VHVL.F2.high.F03_Vh/Vl_VH105-VL43_disulfidel; X = CTSL1-1) |
| ACP465 | mIgG1_Fc-X-IL2-LX-Blocker_(Blocker = VHVL.F2.high.A02_Vh\Vl_A46S; |
| | X = CTSL1-1) |
| ACP466 | mIgG1_Fc-X-IL2-LX-Blocker_(Blocker = VHVL.F2.high.F03_Vh\Vl; X = CTSL1-1) |
| ACP467 | mIgG1_Fc-X-IL2-LX-blocker_(Blocker = |
| | VHVL.F2.high.A02_Vh/Vl_A46S_VH44-VL100_disulfide; X = CTSL1-1) |
| ACP468 | mIgG1_Fc-X-IL2-LX-blocker_(Blocker = |
| | VHVL.F2.high.A02_Vh/Vl_VH105-VL43_disulfidel; X = CTSL1-1) |
| ACP469 | mIgG1_Fc-X-IL2-LX-blocker_(Blocker = |
| | VHVL.F2.high.F03_Vh/Vl_VH44-VL100_disulfidel; X = CTSL1-1) |
| ACP470 | mIgG1_Fc-X-IL2-LX-blocker_(Blocker = |
| | VHVL.F2.high.F03_Vh/Vl_VH105-VL43_disulfidel; X = CTSL1-1) |
| ACP471 | mIgG1_Fc-X-IL2-LX-Heavy_blocker_Fab_(Blocker = MT204_VH-CH1; X = CTSL1-1) |

SEQUENCE TABLE

| SEQ ID | | |
|---|---|---|
| NO. | Name | Sequence |
| 1 | Human | MYRMQLLSCI ALSLALVTNS APTSSSTKKT QLQLEHLLLD LQMILNGINN |
| | IL-2 | YKNPKLTRML TFKFYMPKKA TELKHLQCLE EELKPLEEVL NLAQSKNFHL |
| | | RPRDLISNIN VIVLELKGSE TTFMCEYADE TATIVEFLNR WITFCQSIISTLT |
| 2 | Human | MKWVTFISLL FLFSSAYSRG VFRRDAHKSE VAHRFKDLGE ENFKALVLIA |
| | serum | FAQYLQQCPF EDHVKLVNEV TEFAKTCVAD ESAENCDKSL HTLFGDKLCT |
| | albumin | VATLRETYGE MADCCAKQEP ERNECFLQHK DDNPNLPRLV RPEVDVMCTA |
| | | FHDNEETFLK KYLYEIARRH PYFYAPELLF FAKRYKAAFT ECCQAADKAA |
| | | CLLPKLDELR DEGKASSAKQ GLKCASLQKF GERAFKAWAV ARLSQRFPKA |
| | | EFAEVSKLVT DLTKVHTECC HGDLLECADD RADLAKYICE NQDSISSKLK |
| | | ECCEKPLLEK SHCIAEVEND EMPADLPSLA ADFVGSKDVC KNYAEAKDVF |
| | | LGMFLYEYAR RHPDYSVVLL LRLAKTYETT LEKCCAAADP HECYAKVFDE |
| | | FKPLVEEPQN LIKQNCELFE QLGEYKFQNA LLVRYTKKVP QVSTPTLVEV |
| | | SRNLGKVGSK CCKHPEAKRM PCAEDCLSVF LNQLCVLHEK TPVSDRVTKC |
| | | CTESLVNGRPCFSALEVDETYVPKEFNAETFTFHADICTLSEKERQIKKQTALV |
| | | ELVKHK PKATKEQLKAVMDDFAAFVEKCCKADDKET |
| | | CFAEEGKKLVAASQAALGL |
| 45 | ACP12 | QVQLQESGGGLVQAGGSLRLSCAASGRIFSIDIMSWYRQAPGKQRELVARITRG |
| | (IL2 | GTISYDDSVKGRFTISRDNAKNTVYLQMNSLKPEDTGVYYCNALYGTDYWGKG |
| | fusion | TQVTVSSggggsggggsggggsaptssstkktqlqlehllldlqmilnginnyknpkltrmltfkfympkkatelkhlq |
| | protein) | cleeelkpleevlnlaqsknfhlrprdlisninvivlelkgsettfmceyadetativeflnrwitfcqsiistltSGGPGPAG |
| | | MKGLPGSEVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVYYCTIGGS |
| | | LSVSSQGTLVTVSSggggsggggsggggsEVQLVESGGGLVQPGGSLRLSCAASGFTFS |
| | | SYTLAWVRQAPGKGLEWVAAIDSSSYTYSPDTVRGRFTISRDNAKNSLYLQMNS |
| | | LRAEDTAVYYCARDSNWDALDYWGQGTTVTVSSGGGGSGGGGSGGGGSDIQM |
| | | TQSPSSLSASVGDRVTITCKASQNVGTNVGWYQQKPGKAPKALIYSASFRYSGV |
| | | PSRFSGSGSGTDFTLTISSLQPEDFATYYCQQYYTYPYTFGGGTKVEIKHHHHHH |
| 46 | ACP13 | QVQLQESGGGLVQAGGSLRLSCAASGRIFSIDIMSWYRQAPGKQRELVARITRG |
| | (IL2 | GTISYDDSVKGRFTISRDNAKNTVYLQMNSLKPEDTGVYYCNALYGTDYWGKG |
| | fusion | TQVTVSSggggsggggsggggsEVQLVESGGGLVQPGGSLRLSCAASGFTFSSYTLAW |
| | protein) | VRQAPGKGLEWVAAIDSSSYTYSPDTVRGRFTISRDNAKNSLYLQMNSLRAEDT |
| | | AVYYCARDSNWDALDYWGQGTTVTVSSGGGGSGGGGSGGGGSDIQMTQSPSS |
| | | LSASVGDRVTITCKASQNVGTNVGWYQQKPGKAPKALIYSASFRYSGVPSRFSG |
| | | SGSGTDFTLTISSLQPEDFATYYCQQYYTYPYTFGGGTKVEIKggggsggggsggggsE |
| | | VQLVESGGGLVQPGNSLRLSCAASGFTFSKFGMSWVRQAPGKGLEWVSSISGSG |
| | | RDTLYAESVKGRFTISRDNAKTTLYLQMNSLRPEDTAVYYCTIGGSLSVSSQGTL |
| | | VTVSSSGGPGPAGMKGLPGSaptssstkktqlqlehllldlqmilnginnyknpkltrmltfkfympkkatel |
| | | khlqcleeelkpleevlnlaqsknfhlrprdlisninvivlelkgsettfmceyadetativeflnrwitfcqsiistltHHHHH |
| | | H |
| 47 | ACP14 | EVQLVESGGGLVQPGGSLRLSCAASGFTFSSYTLAWVRQAPGKGLEWVAAIDSS |
| | (IL2 | SYTYSPDTVRGRFTISRDNAKNSLYLQMNSLRAEDTAVYYCARDSNWDALDYW |
| | fusion | GQGTTVTVSSGGGGSGGGGSGGGGSDIQMTQSPSSLSASVGDRVTITCKASQNV |
| | protein) | GTNVGWYQQKPGKAPKALIYSASFRYSGVPSRFSGSGSGTDFTLTISSLQPEDFA |
| | | TYYCQQYYTYPYTFGGGTKVEIKggggsggggsggggsggggsggggsggggsSGGPGPAGM |
| | | KGLPGSaptssstkktqlqlehllldlqmilnginnyknpkltrmltfkfympkkatelkhlqcleeelkpleevlnlaqsk |
| | | nfhlrprdlisninvivlelkgsettfmceyadetativeflnrwitfcqsiistltSGGPGPAGMKGLPGSEVQL |
| | | VESGGGLVQPGNSLRLSCAASGFTFSKFGMSWVRQAPGKGLEWVSSISGSGRDT |
| | | LYAESVKGRFTISRDNAKTTLYLQMNSLRPEDTAVYYCTIGGSLSVSSQGTLVTV |
| | | SSHHHHHH |
| 48 | ACP15 | EVQLVESGGGLVQPGGSLRLSCAASGFTFSSYTLAWVRQAPGKGLEWVAAIDSS |
| | (IL2 | SYTYSPDTVRGRFTISRDNAKNSLYLQMNSLRAEDTAVYYCARDSNWDALDYW |
| | fusion | GQGTTVTVSSGGGGSGGGGSGGGGSDIQMTQSPSSLSASVGDRVTITCKASQNV |
| | protein) | GTNVGWYQQKPGKAPKALIYSASFRYSGVPSRFSGSGSGTDFTLTISSLQPEDFA |
| | | TYYCQQYYTYPYTFGGGTKVEIKggggsggggsggggsggggsggggsggggsEVQLVESGG |
| | | GLVQPGNSLRLSCAASGFTFSKFGMSWVRQAPGKGLEWVSSISGSGRDTLYAES |
| | | VKGRFTISRDNAKTTLYLQMNSLRPEDTAVYYCTIGGSLSVSSQGTLVTVSSgggg |
| | | sggggsggggsSGGPGPAGMKGLPGSaptssstkktqlqlehllldlqmilnginnyknpkltrmltfkfympk |
| | | katelkhlqcleeelkpleevlnlaqsknfhlrprdlisninvivlelkgsettfmceyadetativeflnrwitfcqsiistltHH |
| | | HHHH |
| 49 | ACP16 | aptssstkktqlqlehllldlqmilnginnyknpkltrmltfkfympkkatelkhlqcleeelkpleevlnlaqsknfhlrprdli |
| | (IL2 | sninvivlelkgsettfmceyadetativeflnrwitfcqsiistltSGGPGPAGMKGLPGSEVQLVESGGG |
| | fusion | LVQPGNSLRLSCAASGFTFSKFGMSWVRQAPGKGLEWVSSISGSGRDTLYAESV |
| | protein) | KGRFTISRDNAKTTLYLQMNSLRPEDTAVYYCTIGGSLSVSSQGTLVTVSSggggsg |
| | | gggsggggsggggsggggsggggsSGGPGPAGMKGLPGSEVQLVESGGGLVQPGGSLRLS |
| | | CAASGFTFSSYTLAWVRQAPGKGLEWVAAIDSSSYTYSPDTVRGRFTISRDNAK |
| | | NSLYLQMNSLRAEDTAVYYCARDSNWDALDYWGQGTTVTVSSGGGGSGGGGS |
| | | GGGGSDIQMTQSPSSLSASVGDRVTITCKASQNVGTNVGWYQQKPGKAPKALIY |
| | | SASFRYSGVPSRFSGSGSGTDFTLTISSLQPEDFATYYCQQYYTYPYTFGGGTKV |
| | | EIKHHHHHH |
| 50 | ACP17 | QVQLQESGGGLVQAGGSLRLSCAASGRIFSIDIMSWYRQAPGKQRELVARITRG |
| | (IL2 | GTISYDDSVKGRFTISRDNAKNTVYLQMNSLKPEDTGVYYCNALYGTDYWGKG |
| | fusion | TQVTVSSggggsggggsggggsaptssstkktqlqlehllldlqmilnginnyknpkltrmltfkfympkkatelkhlq |
| | protein) | cleeelkpleevlnlaqsknfhlrprdlisninvivlelkgsettfmceyadetativeflnrwitfcqsiistltSGGPGPAG |
| | | MKGLPGSEVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVYYCTIGGS |
| | | LSVSSQGTLVTVSSggggsggggsggggsggggsggggsggggsSGGPGPAGMKGLPGSEVQ |
| | | LVESGGGLVQPGGSLRLSCAASGFTFSSYTLAWVRQAPGKGLEWVAAIDSSSYT |
| | | YSPDTVRGRFTISRDNAKNSLYLQMNSLRAEDTAVYYCARDSNWDALDYWGQ |
| | | GTTVTVSSGGGGSGGGGSGGGGSDIQMTQSPSSLSASVGDRVTITCKASQNVGT |
| | | NVGWYQQKPGKAPKALIYSASFRYSGVPSRFSGSGSGTDFTLTISSLQPEDFATY |
| | | YCQQYYTYPYTFGGGTKVEIKHHHHHH |
| 51 | ACP18 | QVQLQESGGGLVQAGGSLRLSCAASGRIFSIDIMSWYRQAPGKQRELVARITRG |
| | (IL2 | GTISYDDSVKGRFTISRDNAKNTVYLQMNSLKPEDTGVYYCNALYGTDYWGKG |
| | fusion | TQVTVSSggggsggggsggggsaptssstkktqlqlehllldlqmilnginnyknpkltrmltfkfympkkatelkhlq |
| | protein) | cleeelkpleevlnlaqsknfhlrprdlisninvivlelkgsettfmceyadetativeflnrwitfcqsiistltSGGPGPAG |
| | | MKGLPGSEVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVYYCTIGGS |
| | | LSVSSQGTLVTVSSggggsggggsggggsggggsggggsggggsEVQLVESGGGLVQPGGSLR |
| | | LSCAASGFTFSSYTLAWVRQAPGKGLEWVAAIDSSSYTYSPDTVRGRFTISRDNA |
| | | KNSLYLQMNSLRAEDTAVYYCARDSNWDALDYWGQGTTVTVSSsggpgpagmkgl |
| | | pgsDIQMTQSPSSLSASVGDRVTITCKASQNVGTNVGWYQQKPGKAPKALIYSAS |
| | | FRYSGVPSRFSGSGSGTDFTLTISSLQPEDFATYYCQQYYTYPYTFGGGTKVEIKH |
| | | HHHHH |
| 52 | ACP19 | aptssstkktqlqlehllldlqmilnginnyknpkltrmltfkfympkkatelkhlqcleeelkpleevlnlaqsknfhlrprdli |
| | (IL2 | sninvivlelkgsettfmceyadetativeflnrwitfcqsiistltSGGPGPAGMKGLPGSggggsggggsggggs |
| | fusion | ggggsggggsggggsEVQLVESGGGLVQPGGSLRLSCAASGFTFSSYTLAWVRQAPGK |
| | protein) | GLEWVAAIDSSSYTYSPDTVRGRFTISRDNAKNSLYLQMNSLRAEDTAVYYCAR |
| | | DSNWDALDYWGQGTTVTVSSGGGGSGGGGSGGGGSDIQMTQSPSSLSASVGDR |
| | | VTITCKASQNVGTNVGWYQQKPGKAPKALIYSASFRYSGVPSRFSGSGSGTDFT |
| | | LTISSLQPEDFATYYCQQYYTYPYTFGGGTKVEIKggggsggggsggggsEVQLVESGG |
| | | GLVQPGNSLRLSCAASGFTFSKFGMSWVRQAPGKGLEWVSSISGSGRDTLYAES |
| | | VKGRFTISRDNAKTTLYLQMNSLRPEDTAVYYCTIGGSLSVSSQGTLVTVSSgggg |
| | | sggggsggggsQVQLQESGGGLVQAGGSLRLSCAASGRIFSIDIMSWYRQAPGKQRE |
| | | LVARITRGGTISYDDSVKGRFTISRDNAKNTVYLQMNSLKPEDTGVYYCNALYG |
| | | TDYWGKGTQVTVSSHHHHHH** |
| 53 | ACP20 | aptssstkktqlqlehllldlqmilnginnyknpkltrmltfkfympkkatelkhlqcleeelkpleevlnlaqsknfhlrprdli |
| | (IL2 | sninvivlelkgsettfmceyadetativeflnrwitfcqsiistltSGGPGPAGMKGLPGSEVQLVESGGG |
| | fusion | LVQPGGSLRLSCAASGFTFSSYTLAWVRQAPGKGLEWVAAIDSSSYTYSPDTVR |
| | protein) | GRFTISRDNAKNSLYLQMNSLRAEDTAVYYCARDSNWDALDYWGQGTTVTVS |
| | | SGGGGSGGGGSGGGGSDIQMTQSPSSLSASVGDRVTITCKASQNVGTNVGWYQ |
| | | QKPGKAPKALIYSASFRYSGVPSRFSGSGSGTDFTLTISSLQPEDFATYYCQQYYT |
| | | YPYTFGGGTKVEIKHHHHHH |
| 54 | ACP21 | aptssstkktqlqlehllldlqmilnginnyknpkltrmltfkfympkkatelkhlqcleeelkpleevlnlaqsknfhlrprdli |
| | (IL2 | sninvivlelkgsettfmceyadetativeflnrwitfcqsiistltSGGPGPAGMKGLPGSggggsggggsggggs |
| | fusion | ggggsggggsggggsEVQLVESGGGLVQPGGSLRLSCAASGFTFSSYTLAWVRQAPGK |
| | protein) | GLEWVAAIDSSSYTYSPDTVRGRFTISRDNAKNSLYLQMNSLRAEDTAVYYCAR |
| | | DSNWDALDYWGQGTTVTVSSGGGGSGGGGSGGGGSDIQMTQSPSSLSASVGDR |
| | | VTITCKASQNVGTNVGWYQQKPGKAPKALIYSASFRYSGVPSRFSGSGSGTDFT |
| | | LTISSLQPEDFATYYCQQYYTYPYTFGGGTKVEIKHHHHHH |
| 55 | ACP22 | aptssstkktqlqlehllldlqmilnginnyknpkltrmltfkfympkkatelkhlqcleeelkpleevlnlaqsknfhlrprdli |
| | (IL2 | sninvivlelkgsettfmceyadetativeflnrwitfcqsiistltSGGPGPAGMKGLPGSggggsggggsggggs |
| | fusion | ggggsggggsggggsEVQLVESGGGLVQPGGSLRLSCAASGFTFSSYTLAWVRQAPGK |
| | protein) | GLEWVAAIDSSSYTYSPDTVRGRFTISRDNAKNSLYLQMNSLRAEDTAVYYCAR |
| | | DSNWDALDYWGQGTTVTVSSGGGGSGGGGSGGGGSDIQMTQSPSSLSASVGDR |
| | | VTITCKASQNVGTNVGWYQQKPGKAPKALIYSASFRYSGVPSRFSGSGSGTDFT |
| | | LTISSLQPEDFATYYCQQYYTYPYTFGGGTKVEIKSGGPGPAGMKGLPGSEVQL |
| | | VESGGGLVQPGNSLRLSCAASGFTFSKFGMSWVRQAPGKGLEWVSSISGSGRDT |
| | | LYAESVKGRFTISRDNAKTTLYLQMNSLRPEDTAVYYCTIGGSLSVSSQGTLVTV |
| | | SSggggsggggsggggsQVQLQESGGGLVQAGGSLRLSCAASGRIFSIDIMSWYRQAPG |
| | | KQRELVARITRGGTISYDDSVKGRFTISRDNAKNTVYLQMNSLKPEDTGVYYCN |
| | | ALYGTDYWGKGTQVTVSSHHHHHH |
| 56 | ACP23 | QVQLQESGGGLAQAGGSLSLSCAASGFTVSNSVMAWYRQTPGKQREFVAIINSV |
| | (IL2 | GSTNYADSVKGRFTISRDNAKNTVYLQMNNLKPEDTAVYVCNRNFDRIYWGQG |
| | fusion | TQVTVSSSGGPGPAGMKGLPGSEVQLVESGGGLVQPGGSLRLSCAASGFTFSSY |
| | protein) | TLAWVRQAPGKGLEWVAAIDSSSYTYSPDTVRGRFTISRDNAKNSLYLQMNSLR |
| | | AEDTAVYYCARDSNWDALDYWGQGTTVTVSSGGGGSGGGGSGGGGSDIQMT |
| | | QSPSSLSASVGDRVTITCKASQNVGTNVGWYQQKPGKAPKALIYSASFRYSGVP |
| | | SRFSGSGSGTDFTLTISSLQPEDFATYYCQQYYTYPYTFGGGTKVEIKggggsggggs |
| | | ggggsggggsggggsggggsSGGPGPAGMKGLPGSEVQLVESGGGLVQPGNSLRLSCAA |
| | | SGFTFSKFGMSWVRQAPGKGLEWVSSISGSGRDTLYAESVKGRFTISRDNAKTT |
| | | LYLQMNSLRPEDTAVYYCTIGGSLSVSSQGTLVTVSSSGGPGPAGMKGLPGSapts |
| | | sstkktqlqlehllldlqmilnginnyknpkltrmltfkfympkkatelkhlqcleeelkpleevlnlaqsknfhlrprdlisnin |
| | | vivlelkgsettfmceyadetativeflnrwitfcqsiistltHHHHHH |
| 57 | ACP24 | EVQLVESGGGLVQPGGSLRLSCAASGFTFSSYTLAWVRQAPGKGLEWVAAIDSS |
| | (IL2 | SYTYSPDTVRGRFTISRDNAKNSLYLQMNSLRAEDTAVYYCARDSNWDALDYW |
| | fusion | GQGTTVTVSSGGGGSGGGGSGGGGSDIQMTQSPSSLSASVGDRVTITCKASQNV |
| | protein) | GTNVGWYQQKPGKAPKALIYSASFRYSGVPSRFSGSGSGTDFTLTISSLQPEDFA |
| | | TYYCQQYYTYPYTFGGGTKVEIKSGGPGPAGMKGLPGSaptssstkktqlqlehllldlqmiln |
| | | ginnyknpkltrmltfkfympkkatelkhlqcleeelkpleevlnlaqsknfhlrprdlisninvivlelkgsettfmceyade |
| | | tativeflnrwitfcqsiistltHHHHHH |
| 58 | ACP25 | EVQLVESGGGLVQPGGSLRLSCAASGFTFSSYTLAWVRQAPGKGLEWVAAIDSS |
| | (IL2 | SYTYSPDTVRGRFTISRDNAKNSLYLQMNSLRAEDTAVYYCARDSNWDALDYW |
| | fusion | GQGTTVTVSSGGGGSGGGGSGGGGSDIQMTQSPSSLSASVGDRVTITCKASQNV |
| | protein) | GTNVGWYQQKPGKAPKALIYSASFRYSGVPSRFSGSGSGTDFTLTISSLQPEDFA |
| | | TYYCQQYYTYPYTFGGGTKVEIKggggsggggsggggsggggsggggsggggsSGGPGPAGM |
| | | KGLPGSaptssstkktqlqlehllldlqmilnginnyknpkltrmltfkfympkkatelkhlqcleeelkpleevlnlaqsk |
| | | nfhlrprdlisninvivlelkgsettfmceyadetativeflnrwitfcqsiistltHHHHHH |
| 59 | ACP26 | QVQLQESGGGLVQAGGSLRLSCAASGRIFSIDIMSWYRQAPGKQRELVARITRG |
| | (IL2 | GTISYDDSVKGRFTISRDNAKNTVYLQMNSLKPEDTGVYYCNALYGTDYWGKG |
| | fusion | TQVTVSSggggsggggsggggsaptssstkktqlqlehllldlqmilnginnyknpkltrmltfkfympkkatelkhlq |
| | protein) | cleeelkpleevlnlaqsknfhlrprdlisninvivlelkgsettfmceyadetativeflnrwitfcqsiistltSGGPGPAG |
| | | MKGLPGSEVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVYYCTIGGS |
| | | LSVSSQGTLVTVSSggggsggggsggggsggggsQVQLQQSGAELVRPGTSVKVSCKASG |
| | | YAFTNYLIEWVKQRPGQGLEWIGVINPGSGGTNYNEKFKGKATLTADKSSSTAY |
| | | MQLSSLTSDDSAVYFCARWRGDGYYAYFDVWGAGTTVTVSSggggsggggsggggs |
| | | DIVLTQSPASLAVSLGQRATISCKASQSVDYDGDSYMNWYQQKPGQPPKLLIYA |
| | | ASNLESGIPARFSGSGSGTDFTLNIHPVEEEDAATYYCQQSNEDPYTFGGGTKLEI |
| | | KHHHHHHEPEA |
| 60 | ACP27 | QVQLQESGGGLVQAGGSLRLSCAASGRIFSIDIMSWYRQAPGKQRELVARITRG |
| | (IL2 | GTISYDDSVKGRFTISRDNAKNTVYLQMNSLKPEDTGVYYCNALYGTDYWGKG |
| | fusion | TQVTVSSggggsggggsggggsaptssstkktqlqlehllldlqmilnginnyknpkltrmltfkfympkkatelkhlq |
| | protein) | cleeelkpleevlnlaqsknfhlrprdlisninvivlelkgsettfmceyadetativeflnrwitfcqsiistltSGGPGPAG |
| | | MKGLPGSEVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVYYCTIGGS |
| | | LSVSSQGTLVTVSSggggsggggsggggsggggsDIVLTQSPASLAVSLGQRATISCKASQ |
| | | SVDYDGDSYMNWYQQKPGQPPKLLIYAASNLESGIPARFSGSGSGTDFTLNIHPV |
| | | EEEDAATYYCQQSNEDPYTFGGGTKLEIKggggsggggsggggsQVQLQQSGAELVRP |
| | | GTSVKVSCKASGYAFTNYLIEWVKQRPGQGLEWIGVINPGSGGTNYNEKFKGK |
| | | ATLTADKSSSTAYMQLSSLTSDDSAVYFCARWRGDGYYAYFDVWGAGTTVTV |
| | | SSHHHHHHEPEA |
| 61 | ACP28 | aptssstkktqlqlehllldlqmilnginnyknpkltrmltfkfympkkatelkhlqcleeelkpleevlnlaqsknfhlrprdli |
| | (IL2 | sninvivlelkgsettfmceyadetativeflnrwitfcqsiistltSGGPGPAGMKGLPGSggggsggggsggggs |
| | fusion | ggggsggggsQVQLQQSGAELVRPGTSVKVSCKASGYAFTNYLIEWVKQRPGQGLE |
| | protein) | WIGVINPGSGGTNYNEKFKGKATLTADKSSSTAYMQLSSLTSDDSAVYFCARWR |
| | | GDGYYAYFDVWGAGTTVTVSSggggsggggsggggsDIVLTQSPASLAVSLGQRATIS |
| | | CKASQSVDYDGDSYMNWYQQKPGQPPKLLIYAASNLESGIPARFSGSGSGTDFT |
| | | LNIHPVEEEDAATYYCQQSNEDPYTFGGGTKLEIKggggsggggsggggsEVQLVESGG |
| | | GLVQPGNSLRLSCAASGFTFSKFGMSWVRQAPGKGLEWVSSISGSGRDTLYAES |
| | | VKGRFTISRDNAKTTLYLQMNSLRPEDTAVYYCTIGGSLSVSSQGTLVTVSSgggg |
| | | sggggsggggsQVQLQESGGGLVQAGGSLRLSCAASGRIFSIDIMSWYRQAPGKQRE |
| | | LVARITRGGTISYDDSVKGRFTISRDNAKNTVYLQMNSLKPEDTGVYYCNALYG |
| | | TDYWGKGTQVTVSSHHHHHHEPEA |
| 62 | ACP29 | aptssstkktqlqlehllldlqmilnginnyknpkltrmltfkfympkkatelkhlqcleeelkpleevlnlaqsknfhlrprdli |
| | (IL2 | sninvivlelkgsettfmceyadetativeflnrwitfcqsiistltSGGPGPAGMKGLPGSggggsggggsggggs |
| | fusion | ggggsggggsDIVLTQSPASLAVSLGQRATISCKASQSVDYDGDSYMNWYQQKPGQ |
| | protein) | PPKLLIYAASNLESGIPARFSGSGSGTDFTLNIHPVEEEDAATYYCQQSNEDPYTF |
| | | GGGTKLEIKggggsggggsggggsQVQLQQSGAELVRPGTSVKVSCKASGYAFTNYLI |
| | | EWVKQRPGQGLEWIGVINPGSGGTNYNEKFKGKATLTADKSSSTAYMQLSSLTS |
| | | DDSAVYFCARWRGDGYYAYFDVWGAGTTVTVSSggggsggggsggggsEVQLVESG |
| | | GGLVQPGNSLRLSCAASGFTFSKFGMSWVRQAPGKGLEWVSSISGSGRDTLYAE |
| | | SVKGRFTISRDNAKTTLYLQMNSLRPEDTAVYYCTIGGSLSVSSQGTLVTVSSggg |
| | | gsggggsggggsQVQLQESGGGLVQAGGSLRLSCAASGRIFSIDIMSWYRQAPGKQR |
| | | ELVARITRGGTISYDDSVKGRFTISRDNAKNTVYLQMNSLKPEDTGVYYCNALY |
| | | GTDYWGKGTQVTVSSHHHHHHEPEA |
| 63 | IL2Ra | 10 20 30 40 50 |
| | | MDSYLLMWGL LTFIMVPGCQ AELCDDDPPE IPHATFKAMA YKEGTMLNCE |
| | | 60 70 80 90 100 |
| | | CKRGFRRIKS GSLYMLCTGN SSHSSWDNQC QCTSSATRNT TKQVTPQPEE |
| | | 110 120 130 140 150 |
| | | QKERKTTEMQ SPMQPVDQAS LPGHCREPPP WENEATERIY HFVVGQMVYY |
| | | 160 170 180 190 200 |
| | | QCVQGYRALH RGPAESVCKM THGKTRWTQP QLICTGEMET SQFPGEEKPQ |
| | | 210 220 230 240 250 |
| | | ASPEGRPESE TSCLVTTTDF QIQTEMAATM ETSIFTTEYQ VAVAGCVFLL |
| | | 260 270 |
| | | ISVLLLSGLT WQRRQRKSRR TI |
| 64 | IL2Rb | 10 20 30 40 50 |
| | | MAAPALSWRL PLLILLLPLA TSWASAAVNG TSQFTCFYNS RANISCVWSQ |
| | | 60 70 80 90 100 |
| | | DGALQDTSCQ VHAWPDRRRW NQTCELLPVS QASWACNLIL GAPDSQKLTT |
| | | 110 120 130 140 150 |
| | | VDIVTLRVLC REGVRWRVMA IQDFKPFENL RLMAPISLQV VHVETHRCNI |
| | | 160 170 180 190 200 |
| | | SWEISQASHY FERHLEFEAR TLSPGHTWEE APLLTLKQKQ EWICLETLTP |
| | | 210 220 230 240 250 |
| | | DTQYEFQVRV KPLQGEFTTW SPWSQPLAFR TKPAALGKDT IPWLGHLLVG |
| | | 260 270 280 290 300 |
| | | LSGAFGFIIL VYLLINCRNT GPWLKKVLKC NTPDPSKFFS QLSSEHGGDV |
| | | 310 320 330 340 350 |
| | | QKWLSSPFPS SSFSPGGLAP EISPLEVLER DKVTQLLLQQ DKVPEPASLS |
| | | 360 370 380 390 400 |
| | | SNHSLTSCFT NQGYFFFHLP DALEIEACQV YFTYDPYSEE DPDEGVAGAP |
| | | 410 420 430 440 450 |
| | | TGSSPQPLQP LSGEDDAYCT FPSRDDLLLF SPSLLGGPSP PSTAPGGSGA |
| | | 460 470 480 490 500 |
| | | GEERMPPSLQ ERVPRDWDPQ PLGPPTPGVP DLVDFQPPPE LVLREAGEEV |
| | | 510 520 530 540 550 |
| | | PDAGPREGVS FPWSRPPGQG EFRALNARLP LNTDAYLSLQ ELQGQDPTHL |
| | | V |
| 65 | IL2Rg | 10 20 30 40 50 |
| | | MLKPSLPFTS LLFLQLPLLG VGLNTTILTP NGNEDTTADF FLTTMPTDSL |
| | | 60 70 80 90 100 |
| | | SVSTLPLPEV QCFVFNVEYM NCTWNSSSEP QPTNLTLHYW YKNSDNDKVQ |
| | | 110 120 130 140 150 |
| | | KCSHYLFSEE ITSGCQLQKK EIHLYQTFVV QLQDPREPRR QATQMLKLQN |
| | | 160 170 180 190 200 |
| | | LVIPWAPENL TLHKLSESQL ELNWNNRFLN HCLEHLVQYR TDWDHSWTEQ |
| | | 210 220 230 240 250 |
| | | SVDYRHKFSL PSVDGQKRYT FRVRSRFNPL CGSAQHWSEW SHPIHWGSNT |
| | | 260 270 280 290 300 |
| | | SKENPFLFAL EAVVISVGSM GLIISLLCVY FWLERTMPRI PTLKNLEDLV |
| | | 310 320 330 340 350 |
| | | TEYHGNFSAW SGVSKGLAES LQPDYSERLC LVSEIPPKGG ALGEGPGASP |
| | | 360 |
| | | CNQHSPYWAP PCYTLKPET |
| 66 | ACP04 | iwelkkdvyvveldwypdapgemvvltcdtpeedgitwtldqssevlgsgktliqvkefgdagqytchkggevlshslll |
| | (human | lhkkedgiwstdilkdqkepknktflrceaknysgrftcwwlttistdltfsvkssrgssdpqgvtcgaatlsaervrgdnkey |
| | p40/murine | eysvecqedsacpaaeeslpievmvdavhklkyenytssffirdiikpdppknlqlkplknsrqvevsweypdtwstphs |
| | p35 | yfsltfcvqvqgkskrekkdrvftdktsatvicrknasisvraqdryyssswsewasvpcsggggsggggsggggsrvipv |
| | IL12 | sgparclsqsrnllkttddmvktareklkhysctaedidheditrdqtstlktclplelhknesclatretssttrgsclppqktslm |
| | fusion | mtlclgsiyedlkmyqtefqainaalqnhnhqqiildkgmlvaidelmqslnhngetlrqkppvgeadpyrvkmklcillh |
| | protein) | afstrvvtinrymgylssaHHHHHH |
| 67 | ACP05 | iwelkkdvyvveldwypdapgemvvltcdtpeedgitwtldqssevlgsgktltiqvkefgdagqytchkggevlshslll |
| | (human | lhkkedgiwstdilkdqkepknktflrceaknysgrftcwwlttistdltsvkssrgssdpqgvtcgaatlsaervrgdnkey |
| | p40/murine | eysvecqedsacpaaeeslpievmvdavhklkyenytssffirdiikpdppknlqlkplknsrqvevsweypdtwstphs |
| | p35 | yfsltfcvqvqgkskrekkdrvftdktsatvicrknasisvraqdryyssswsewasvpcsggggsggggsggggsrnlpv |
| | IL12 | atpdpgmfpclhhsqnllravsnmlqkarqtlefypctseeidheditkdktstveaclpleltknesclnsretsfitngsclas |
| | fusion | rktsfmmalclssiyedlkmyqvefktmnakllmdpkrqifldqnmlavidelmqalnfnsetypqkssleepdfyktki |
| | protein) | klcillhafriravtidrvmsylnasHHHHHH |
| 68 | ACP06 | QSVLTQPPSVSGAPGQRVTISCSGSRSNIGSNTVKWYQQLPGTAPKLLIYYNDQR |
| | (human | PSGVPDRFSGSKSGTSASLAITGLQAEDEADYYCQSYDRYTHPALLFGTGTKVT |
| | p40/murine | VLggggsggggsggggsQVQLVESGGGVVQPGRSLRLSCAASGFTFSSYGMHWVRQA |
| | p35 | PGKGLEWVAFIRYDGSNKYYADSVKGRFTISRDNSKNTLYLQMNSLRAEDTAV |
| | IL12 | YYCKTHGSHDNWGQGTMVTVSSggggsggggsggggsggggsggggsggggsSGGPGPAG |
| | fusion | MKGLPGSiwelkkdvyvveldwypdapgemvvltcdtpeedgitwtldqssevlgsgktltiqvkefgdagqytch |
| | protein) | kggevlshsllllhkkedgiwstdilkdqkepknktflrceaknysgrftcwwlttistdltfsvkssrgssdpqgvtcgaatls |
| | | aervrgdnkeyeysvecqedsacpaaeeslpievmvdavhklkyenytssffirdiikpdppknlqlkplknsrqvevsw |
| | | eypdtwstphsyfsltfcvqvqgkskrekkdrvftdktsatvicrknasisvraqdryyssswsewasvpcsggggsgggg |
| | | sggggsrvipvsgparclsqsmllkttddmvktareklkhysctaedidheditrdqtstlktclplelhknesclatretssttr |
| | | gsclppqktslmmtlclgsiyedlkmyqtefqainaalqnhnhqqiildkgmlvaidelmqslnhngetlrqkppvgead |
| | | pyrvkmklcillhafstrvvtinrvmgylssaSGGPGPAGMKGLPGSEVQLVESGGGLVQPGNSL |
| | | RLSCAASGFTFSKFGMSWVRQAPGKGLEWVSSISGSGRDTLYAESVKGRFTISR |
| | | DNAKTTLYLQMNSLRPEDTAVYYCTIGGSLSVSSQGTLVTVSSHHHHHHEPEA |
| 69 | ACP07 | QSVLTQPPSVSGAPGQRVTISCSGSRSNIGSNTVKWYQQLPGTAPKLLIYYNDQR |
| | (human | PSGVPDRFSGSKSGTSASLAITGLQAEDEADYYCQSYDRYTHPALLFGTGTKVT |
| | p40/murine | VLggggsggggsggggsQVQLVESGGGVVQPGRSLRLSCAASGFTFSSYGMHWVRQA |
| | p35 | PGKGLEWVAFIRYDGSNKYYADSVKGRFTISRDNSKNTLYLQMNSLRAEDTAV |
| | IL12 | YYCKTHGSHDNWGQGTMVTVSSggggsggggsggggsggggsggggsggggsSGGPGPAG |
| | fusion | MKGLPGSiwelkkdvyvveldwypdapgemvvltcdtpeedgitwtldqssevlgsgktltiqvkefgdagqytch |
| | protein) | kggevlshsllllhkkedgiwstdilkdqkepknktflrceaknysgrftcwwlttistdltfsvkssrgssdpqgvtcgaatls |
| | | aervrgdnkeyeysvecqedsacpaaeeslpievmvdavhklkyenytssffirdiikpdppknlqlkplknsrqvevsw |
| | | eypdtwstphsyfsltfcvqvqgkskrekkdrvftdktsatvicrknasisvraqdryyssswsewasvpcsggggsgggg |
| | | sggggsrvipvsgparclsqsrnllkttddmvktareklkhysctaedidheditrdqtstlktclplelhknesclatretssttr |
| | | gsclppqktslmmtlclgsiyedlkmyqtefqainaalqnhnhqqiildkgmlvaidelmqslnhngetlrqkppvgead |
| | | pyrvkmklcillhafstrvvtinrvmgylssaSGGPGPAGMKGLPGSEVQLVESGGGLVQPGNSL |
| | | RLSCAASGFTFSKFGMSWVRQAPGKGLEWVSSISGSGRDTLYAESVKGRFTISR |
| | | DNAKTTLYLQMNSLRPEDTAVYYCTIGGSLSVSSQGTLVTVSSggggsggggsggggs |
| | | QVQLQESGGGLAQAGGSLSLSCAASGFTVSNSVMAWYRQTPGKQREFVAIINSV |
| | | GSTNYADSVKGRFTISRDNAKNTVYLQMNNLKPEDTAVYVCNRNFDRIYWGQG |
| | | TQVTVSSHHHHHHEPEA |
| 70 | ACP08 | QVQLQESGGGLAQAGGSLSLSCAASGFTVSNSVMAWYRQTPGKQREFVAIINSV |
| | (human | GSTNYADSVKGRFTISRDNAKNTVYLQMNNLKPEDTAVYVCNRNFDRIYWGQG |
| | p40/murine | TQVTVSSggggsggggsggggsQSVLTQPPSVSGAPGQRVTISCSGSRSNIGSNTVKWY |
| | p35 | QQLPGTAPKLLIYYNDQRPSGVPDRFSGSKSGTSASLAITGLQAEDEADYYCQSY |
| | IL12 | DRYTHPALLFGTGTKVTVLggggsggggsggggsQVQLVESGGGVVQPGRSLRLSCAA |
| | fusion | SGFTFSSYGMHWVRQAPGKGLEWVAFIRYDGSNKYYADSVKGRFTISRDNSKN |
| | protein) | TLYLQMNSLRAEDTAVYYCKTHGSHDNWGQGTMVTVSSggggsggggsggggsggggs |
| | | ggggsggggsSGGPGPAGMKGLPGSiwelkkdvyvveldwypdapgemvvltcdtpeedgitwtldqsse |
| | | vlgsgktltiqvkefgdagqytchkggevlshsllllhkkedgiwstdilkdqkepknktflrceaknysgrftcwwlttistdl |
| | | tfsvkssrgssdpqgvtcgaatlsaervrgdnkeyeysvecqedsacpaaeeslpievmvdavhklkyenytssffirdiik |
| | | pdppknlqlkplknsrqvevsweypdtwstphsyfsltfcvqvqgkskrekkdrvftdktsatvicrknasisvraqdryys |
| | | sswsewasvpcsggggsggggsggggsrvipvsgparclsqsrnllkttddmvktareklkhysctaedidheditrdqtst |
| | | lktclplelhknesclatretssttrgsclppqktslmmtlclgsiyedlkmyqtefqainaalqnhnhqqiildkgmlvaidel |
| | | mqslnhngetlrqkppvgeadpyrvkmklcillhafstrvvtinrvmgylssaSGGPGPAGMKGLPGSEVQ |
| | | LVESGGGLVQPGNSLRLSCAASGFTFSKFGMSWVRQAPGKGLEWVSSISGSGRD |
| | | TLYAESVKGRFTISRDNAKTTLYLQMNSLRPEDTAVYYCTIGGSLSVSSQGTLVT |
| | | VSSHHHHHHEPEA |
| 71 | ACP09 | EVQLVESGGGLVQPGNSLRLSCAASGFTFSKFGMSWVRQAPGKGLEWVSSISGS |
| | (human | GRDTLYAESVKGRFTISRDNAKTTLYLQMNSLRPEDTAVYYCTIGGSLSVSSQGT |
| | p40/murine | LVTVSSggggsggggsggggsQSVLTQPPSVSGAPGQRVTISCSGSRSNIGSNTVKWYQ |
| | p35 | QLPGTAPKLLIYYNDQRPSGVPDRFSGSKSGTSASLAITGLQAEDEADYYCQSYD |
| | IL12 | RYTHPALLFGTGTKVTVLggggsggggsggggsQVQLVESGGGVVQPGRSLRLSCAAS |
| | fusion | GFTFSSYGMHWVRQAPGKGLEWVAFIRYDGSNKYYADSVKGRFTISRDNSKNT |
| | protein) | LYLQMNSLRAEDTAVYYCKTHGSHDNWGQGTMVTVSSggggsggggsggggsggggsg |
| | | gggsggggsSGGPGPAGMKGLPGSiwelkkdvyvveldwypdapgemvvltcdtpeedgitwtldqssevl |
| | | gsgktltiqvkefgdagqytchkggevlshsllllhkkedgiwstdilkdqkepknktflrceaknysgrftcwwlttistdltf |
| | | svkssrgssdpqgvtcgaatlsaervrgdnkeyeysvecqedsacpaaeeslpievmvdavhklkyenytssffirdiikpd |
| | | ppknlqlkplknsrqvevsweypdtwstphsyfsltfcvqvqgkskrekkdrvftdktsatvicrknasisvraqdryysss |
| | | wsewasvpcsggggsggggsggggsrvipvsgparclsqsrnllkttddmvktareklkhysctaedidheditrdqtstlk |
| | | tclplelhknesclatretssttrgsclppqktslmmtlclgsiyedlkmyqtefqainaalqnhnhqqiildkgmlvaidelm |
| | | qslnhngetlrqkppvgeadpyrvkmklcillhafstrvvtinrvmgylssaHHHHHHEPEA |
| 72 | ACP10 | EVQLVESGGGLVQPGNSLRLSCAASGFTFSKFGMSWVRQAPGKGLEWVSSISGS |
| | (human | GRDTLYAESVKGRFTISRDNAKTTLYLQMNSLRPEDTAVYYCTIGGSLSVSSQGT |
| | p40/murine | LVTVSSSGGPGPAGMKGLPGSiwelkkdvyvveldwypdapgemvvltcdtpeedgitwtldqssevlg |
| | p35 | sgktltiqvkefgdagqytchkggevlshsllllhkkedgiwstdilkdqkepknktflrceaknysgrftcwwlttistdltfs |
| | IL12 | vkssrgssdpqgvtcgaatlsaervrgdnkeyeysvecqedsacpaaeeslpievmvdavhklkyenytssffirdiikpd |
| | fusion | ppknlqlkplknsrqvevsweypdtwstphsyfsltfcvqvqgkskrekkdrvftdktsatvicrknasisvraqdryysss |
| | protein) | wsewasvpcsggggsggggsggggsrvipvsgparclsqsrnllkttddmvktareklkhysctaedidheditrdqtstlk |
| | | tclplelhknesclatretssttrgsclppqktslmmtlclgsiyedlkmyqtefqainaalqnhnhqqiildkgmlvaidelm |
| | | qslnhngetlrqkppvgeadpyrvkmklcillhafstrvvtinrvmgylssaSGGPGPAGMKGLPGSggggsg |
| | | gggsggggsggggsggggsggggsQSVLTQPPSVSGAPGQRVTISCSGSRSNIGSNTVKWY |
| | | QQLPGTAPKLLIYYNDQRPSGVPDRFSGSKSGTSASLAITGLQAEDEADYYCQSY |
| | | DRYTHPALLFGTGTKVTVLggggsggggsggggsQVQLVESGGGVVQPGRSLRLSCAA |
| | | SGFTFSSYGMHWVRQAPGKGLEWVAFIRYDGSNKYYADSVKGRFTISRDNSKN |
| | | TLYLQMNSLRAEDTAVYYCKTHGSHDNWGQGTMVTVSSHHHHHHEPEA |
| 73 | ACP11 | iwelkkdvyvveldwypdapgemvvltcdtpeedgitwtldqssevlgsgktltiqvkefgdagqytchkggevlshslll |
| | (human | lhkkedgiwstdilkdqkepknktftlrceaknysgrftcwwlttistdltfsvkssrgssdpqgvtcgaatlsaervrgdnkey |
| | p40/murine | eysvecqedsacpaaeeslpievmvdavhklkyenytssffirdiikpdppknlqlkplknsrqvevsweypdtwstphs |
| | p35 | yfsltfcvqvqgkskrekkdrvftdktsatvicrknasisvraqdryyssswsewasvpcsggggsggggsggggsrvipv |
| | IL12 | sgparclsqsrnllkttddmvktareklkhysctaedidheditrdqtstlktclplelhknesclatretssttrgsclppqktslm |
| | fusion | mtlclgsiyedlkmyqtefqainaalqnhnhqqiildkgmlvaidelmqslnhngetlrqkppvgeadpyrvkmklcillh |
| | protein) | afstryvtinrvmgylssaSGGPGPAGMKGLPGSggggsggggsggggsggggsggggsggggsQSVLT |
| | | QPPSVSGAPGQRVTISCSGSRSNIGSNTVKWYQQLPGTAPKWYYNDQRPSGVP |
| | | DRFSGSKSGTSASLAITGLQAEDEADYYCQSYDRYTHPALLFGTGTKVTVLggggs |
| | | ggggsggggsQVQLVESGGGVVQPGRSLRLSCAASGFTFSSYGMHWVRQAPGKGLE |
| | | WVAFIRYDGSNKYYADSVKGRFTISRDNSKNTLYLQMNSLRAEDTAVYYCKTH |
| | | GSHDNWGQGTMVTVSSggggsggggsggggsEVQLVESGGGLVQPGNSLRLSCAASG |
| | | FTFSKFGMSWVRQAPGKGLEWVSSISGSGRDTLYAESVKGRFTISRDNAKTTLY |
| | | LQMNSLRPEDTAVYYCTIGGSLSVSSQGTLVTVSSHEIREIHHEPEA |
| 74 | IL12 p40 | 10 20 30 40 50 |
| | human | MCHQQLVISW FSLVFLASPL VAIWELKKDY YVVELDWYPD APGEMVVLTC |
| | (Uniprot | 60 70 80 90 100 |
| | Accession | DTPEEDGITW TLDQSSEVLG SGKTLTIQVK EFGDAGQYTC HKGGEVLSHS |
| | No. | 110 120 130 140 150 |
| | P29460) | LLLLHKKEDG IWSTDILKDQ KEPKNKTFLR CEAKNYSGRF TCWWLTTIST |
| | | 160 170 180 190 200 |
| | | DLTFSVKSSR GSSDPQGVTC GAATLSAERV RGDNKEYEYS VECQEDSACP |
| | | 210 220 230 240 250 |
| | | AAEESLPIEV MVDAVHKLKY ENYTSSFFIR DIIKPDPPKN LQLKPLKNSR |
| | | 260 270 280 290 300 |
| | | QVEVSWEYPD TWSTPHSYFS LTFCVQVQGK SKREKKDRVF TDKTSATVIC |
| | | 310 320 |
| | | RKNASISVRA QDRYYSSSWS EWASVPCS |
| 75 | IL12 p35 | 10 20 30 40 50 |
| | mouse | MCQSRYLLFL ATLALLNHLS LARVIPVSGP ARCLSQSRNL LKTTDDMVKT |
| | (Uniprot | 60 70 80 90 100 |
| | Accession | AREKLKHYSC TAEDIDHEDI TRDQTSTLKT CLPLELHKNE SCLATRETSS |
| | No. | 110 120 130 140 150 |
| | P43431) | TTRGSCLPPQ KTSLMMTLCL GSIYEDLKMY QTEFQAINAA LQNHNHQQII |
| | | 160 170 180 190 200 |
| | | LDKGMLVAID ELMQSLNHNG ETLRQKPPVG EADPYRVKMK LCILLHAFST |
| | | 210 |
| | | RVVTINRVMG YLSSA |
| 76 | IL12Rb- | 10 20 30 40 50 |
| | 2 | MAHTFRGCSL AFMFIITELL IKAKIDACKR GDVTVKPSHY ILLGSIYNIT |
| | | 60 70 80 90 100 |
| | | CSLFPROGCF HYSRRNKLIL YKFDRRINEH HGHSLNSQVT GLPLGTTLFV |
| | | 110 120 130 140 150 |
| | | CKLACINSDE IQICGAFIFV GVAREQRQNL SCIQKGEQGT VACTQERGRD |
| | | 160 170 180 190 200 |
| | | IHLYTEYTOQ LSGPKNLTWQ KQCKDIYCDY LDFGINLTPE SPESQFTAKY |
| | | 210 220 230 240 250 |
| | | TAYMELGSES SLPSTFTFLD IVRPLPFWDI RIKFQKASVS RCTLYWRDEG |
| | | 260 270 280 290 300 |
| | | LVLLNRLRYR PSNSRLWNMV NVTKAKGRHD LLDLKPFTEY EFQISSKLHL |
| | | 310 320 330 340 350 |
| | | YKGSWSDWSE SLPAQTPEEE PTGMLDVWYM KRHIDYSRQQ ISLFWWQLSV |
| | | 360 370 380 390 400 |
| | | SEARGKILHY QVTLQELTGG KAMTQNITGH ISWTIVIRRT GNWAVAVSAA |
| | | 410 420 430 440 450 |
| | | NSKGSSLPTR INIMNLCEAG LLAPRQVSAN SEGMDNILVI KQPPRKDRSA |
| | | 460 470 480 490 500 |
| | | VQEYVVENRE LHPGGDIQYR LNWLRSRPYN VSALISENIK SYICYEIRVY |
| | | 510 520 530 540 550 |
| | | ALSGDQGGCS SILGNSKHKA PLSGPHINAI IEEKGSILIS WNSIRVQEQM |
| | | 560 570 580 590 600 |
| | | GCLLHYRIYW KERDSNSQPQ LCEIPYRYSQ NSHPINSLQP RVIYVLWMTA |
| | | 610 620 630 640 650 |
| | | LTAAGESSHG NEREECLQGK ANWMAFVARS ICIAIIMVGI FSTHYFQQKV |
| | | 660 670 680 690 700 |
| | | FVLLAALRPQ WCSREIPDPA NSICAKKYPI AEEKTQLPLQ RLLIDWPTPE |
| | | 710 720 730 740 750 |
| | | DPEPLVISEV LHQVIPVFRH PPCSNWPQRE KGIQGHQASE KDMMHSASSR |
| | | 760 770 780 790 800 |
| | | PPPRALQAES PQLVDLYKVL ESRGSDPKRE NPACPWTVLR AGDLPTHDGY |
| | | 810 820 830 840 850 |
| | | LPSNIDDLPS HEAPLADSLE ELEPQHISLS VFPSSSLHPL TFSCGDKLTL |
| | | 860 |
| | | DQLKMRCDSL ML |
| 77 | IL12Rb- | 10 20 30 40 50 |
| | 1 | MEPLVTWVVP LLFLFLLSRQ GAACRISECC FQDPPYPDAD SGSASGPRDL |
| | | 60 70 80 90 100 |
| | | RCYRISSDRY ECSWQYEGPT AGVSHFLRCC LSSGRCCYFA AGSATRLQFS |
| | | 110 120 130 140 150 |
| | | DQAGVSVLYT VTLWVESWAR NQTEKSPEVT LQLYNSVKYE PPLGDIKVSK |
| | | 160 170 180 190 200 |
| | | LAGQLRMEWE TPDNQVGAEV QFRHRIPSSP WKLGDCGPQD DDTESCLCPL |
| | | 210 220 230 240 250 |
| | | EMNVAQEFQL RRRQLGSQGS SWSKWSSPVC VPPENPPQPQ VRFSVEQLGQ |
| | | 260 270 280 290 300 |
| | | DGRRRLILKE QPTQLELPEG CQGLAPGTEV TYRLQLHMLS CPCKAKATRT |
| | | 310 320 330 340 350 |
| | | LHLGKMPYLS GAAYNVAVIS SNQFGPGLNQ TWHTPADTHT EPVALNISVG |
| | | 360 370 380 390 400 |
| | | INGTTMYWPA RAQSMTYCIE WQPVGQDGGL ATCSLTAPQD PDPAGMATYS |
| | | 410 420 430 440 450 |
| | | WSRESGAMGQ EKCYYITIFA SAHPEKLTLW STVLSTYHFG GNASAAGTPH |
| | | 460 470 480 490 500 |
| | | HVSVKNHSLD SVSVDWAPSL LSTCPGVLKE YVVRCRDEDS KQVSEHPVQP |
| | | 510 520 530 540 550 |
| | | TETQVTLSGL RAGVAYTVQV RADTAWLRGV WSQPQRFSIE VQVSDWLIFF |
| | | 560 570 580 590 600 |
| | | ASLGSFLSIL LVGVLGYLGL NRAARHLCPP LPTPCASSAI EFPGGKETWQ |
| | | 610 620 630 640 650 |
| | | WINPVDFQEE ASLQEALVVE MSWDKGERTE PLEKTELPEG APELALDTEL |
| | | 660 |
| | | SLEDGDRCKA KM |
| 78 | IL-12 | 10 20 30 40 50 |
| | p35 | MCHQQLVISW FSLVFLASPL VAIWELKKDV YVVELDWYPD APGEMVVLTC |
| | human | 60 70 80 90 100 |
| | (Uniprot | DTPEEDGITW TLDQSSEVLG SGKTLTIQVK EFGDAGQYTC HKGGEVLSHS |
| | accession | 110 120 130 140 150 |
| | no. | LLLLHKKEDG IWSTDILKDQ KEPKNKTFLR CEAKNYSGRF TCWWLTTIST |
| | P29459) | 160 170 180 190 200 |
| | | DLTFSVKSSR GSSDPQGVTC GAATLSAERV RGDNKEYEYS VECQEDSACP |
| | | 210 220 230 240 250 |
| | | AAEESLPIEV MVDAVHKLKY ENYTSSFFIR DIIKPDPPKN LQLKPLKNSR |
| | | 260 270 280 290 300 |
| | | QVEVSWEYPD TWSTPHSYFS LTFCVQVQGK SKREKKDRVF TDKTSATVIC |
| | | 310 320 |
| | | RKNASISVRA QDRYYSSSWS EWASVPCS |
| 79 | IL-12 | 10 20 30 40 50 |
| | p40 | MCPQKLTISW FAIVLLVSPL MAMWELEKDV YVVEVDWTPD APGETVNLTC |
| | mouse | 60 70 80 90 100 |
| | (Uniprot | DTPEEDGITW TSDQRHGVIG SGKTLTITVK EFLDAGQYTC HKGGETLSHS |
| | accession | 110 120 130 140 150 |
| | no. | HLLLHKKENG IWSTEILKNF KNKTFLKCEA PNYSGRFTCS WLVQRNMDLK |
| | P43432) | 160 170 180 190 200 |
| | | FNIKSSSSSP DSRAVTCGMA SLSAEKVTLD QRDYEKYSVS CQEDVTCPTA |
| | | 210 220 230 240 250 |
| | | EETLPIELAL EARQQNKYEN YSTSFFIRDI IKPDPPKNLQ MKPLKNSQVE |
| | | 260 270 280 290 300 |
| | | VSWEYPDSWS TPHSYFSLKF FVRIQRKKEK MKETEEGCNQ KGAFLVEKTS |
| | | 310 320 330 |
| | | TEVQCKGGNV CVQAQDRYYN SSCSKWACVP CRVRS |
| 80 | ACP01 | EVQLVESGGGLVQPGNSLRLSCAASGFTFSKFGMSWVRQAPGKGLEWVSSISGS |
| | (mouse | GRDTLYAESVKGRFTISRDNAKTTLYLQMNSLRPEDTAVYYCTIGGSLSVSSQGT |
| | IFNg | LVTVSSSGGPGPAGMKGLPGShgtviesleslnnyfnssgidveekslfldiwrnwqkdgdmkilqsqiisf |
| | fusion | ylrlfevlkdnqaisnnisvieshlittffsnskakkdafmsiakfevnnpqvqrqafnelirvvhqllpesslrkrkrsrcSG |
| | protein) | GPGPAGMKGLPGSEVQLVESGGGLVQPGNSLRLSCAASGFTFSKFGMSWVRQA |
| | | PGKGLEWVSSISGSGRDTLYAESVKGRFTISRDNAKTTLYLQMNSLRPEDTAVY |
| | | YCTIGGSLSVSSQGTLVTVSSHHHHHH |
| 81 | ACP02 | EVQLVESGGGLVQPGNSLRLSCAASGFTFSKFGMSWVRQAPGKGLEWVSSISGS |
| | (mouse | GRDTLYAESVKGRFTISRDNAKTTLYLQMNSLRPEDTAVYYCTIGGSLSVSSQGT |
| | IFNg | LVTVSSSGGPGPAGMKGLPGShgtviesleslnnyfnssgidveekslfldiwrnwqkdgdmkilqsqiisf |
| | fusion | ylrlfevlkdnqaisnnisvieshlittffsnskakkdafmsiakfevnnpqvqrqafnelirvvhqllpesslrkrkrsrcSG |
| | protein) | GPGPAGMKGLPGShgtviesleslnnyfnssgidveekslfldiwrnwqkdgdmkilqsqiisfylrlfevlkdnq |
| | | aisnnisvieshlittffsnskakkdafmsiakfevnnpqvqrqafnelirvvhqllpesslrkrkrsrcSGGPGPAGM |
| | | KGLPGSEVQLVESGGGLVQPGNSLRLSCAASGFTFSKFGMSWVRQAPGKGLEW |
| | | VSSISGSGRDTLYAESVKGRFTISRDNAKTTLYLQMNSLRPEDTAVYYCTIGGSL |
| | | SVSSQGTLVTVSSHHHHHH |
| 82 | ACP03 | EVQLVESGGGLVQPGNSLRLSCAASGFTFSKFGMSWVRQAPGKGLEWVSSISGS |
| | (mouse | GRDTLYAESVKGRFTISRDNAKTTLYLQMNSLRPEDTAVYYCTIGGSLSVSSQGT |
| | IFNg | LVTVSSSGGPGPAGMKGLPGShgtviesleslnnyfnssgidveekslfldiwrnwqkdgdmkilqsqiisf |
| | fusion | ylrlfevlkdnqaisnnisvieshlittffsnskakkdafmsiakfevnnpqvqrqafnelirvvhqllpesslrkrkrsrcggg |
| | protein) | gsggggsggggshgtviesleslnnyfnssgidveekslfidiwrnwqkdgdmkilqsqiisfylrlfevlkdnqaisnnisv |
| | | ieshlittffsnskakkdafmsiakfevnnpqvqrqafnelirvvhqllpesslrkrkrsrcSGGPGPAGMKGLPGS |
| | | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSHHHHHH |
| 83 | Human | 10 20 30 40 50 |
| | IFN-g | MKYTSYILAF QLCIVLGSLG CYCQDPYVKE AENLKKYFNA GHSDVADNGT |
| | (Uniprot | 60 70 80 90 100 |
| | Accession | LFLGILKNWK EESDRKIMQS QIVSFYFKLF KNFKDDQSIQ KSVETIKEDM |
| | No. | 110 120 130 140 150 |
| | P01579) | NVKFFNSNKK KRDDFEKLTN YSVTDLNVQR KAIHELIQVM AELSPAAKTG |
| | | 160 |
| | | KRKRSQMLFR GRRASQ |
| 84 | Mouse | 10 20 30 40 50 |
| | IFN-g | MNATHCILAL QLFLMAVSGC YCHGTVIESL ESLNNYFNSS GIDVEEKSLF |
| | (Uniprot | 60 70 80 90 100 |
| | Accession | LDIWRNWQKD GDMKILQSQI ISFYLRLFEV LKDNQAISNN ISVIESHLIT |
| | No. | 110 120 130 140 150 |
| | P01580) | TFFSNSKAKK DAFMSIAKFE VNNPQVQRQA FNELIRVVHQ LLPESSLRKR |
| | | KRSRC |
| 85 | ACP30 | mdmrvpaqllgllllwlrgarcEVQLVESGGGLVQPGNSLRLSCAASGFTFSKFGMSWVRQ |
| | (mouse | APGKGLEWVSSISGSGRDTLYAESVKGRFTISRDNAKTTLYLQMNSLRPEDTAV |
| | IFNg | YYCTIGGSLSVSSQGTLVTVSSSGGPGPAGMKGLPGShgtviesleslnnyfnssgidveekslfl |
| | fusion | diwrnwqkdgdmkilqsqiisfylrlfevlkdnqaisnnisvieshlittffsnskakkdafmsiakfevnnpqvqrqafnel |
| | protein) | irvvhqllpesslrkrkrsrcSGGPGPAGMKGLPGSEVQLVESGGGLVQPGNSLRLSCAASG |
| | | FTFSKFGMSWVRQAPGKGLEWVSSISGSGRDTLYAESVKGRFTISRDNAKTTLY |
| | | LQMNSLRPEDTAVYYCTIGGSLSVSSQGTLVTVSSSGGPGPAGMKGLPGShgtvies |
| | | leslnnyfnssgidveekslfldiwrnwqkdgdmkilqsqiisfylrlfevlkdnqaisnnisvieshlittffsnskakkdaf |
| | | msiakfevnnpqvqrqafnelirvvhqllpesslrkrkrsrcSGGPGPAGMKGLPGSEVQLVESGGGL |
| | | VQPGNSLRLSCAASGFTFSKFGMSWVRQAPGKGLEWVSSISGSGRDTLYAESVK |
| | | GRFTISRDNAKTTLYLQMNSLRPEDTAVYYCTIGGSLSVSSQGTLVTVSSHHHHH |
| | | H |
| 86 | ACP31 | EVQLVESGGGLVQPGNSLRLSCAASGFTFSKFGMSWVRQAPGKGLEWVSSISGS |
| | (mouse | GRDTLYAESVKGRFTISRDNAKTTLYLQMNSLRPEDTAVYYCTIGGSLSVSSQGT |
| | IFNa1 | LVTVSSSGGPGPAGMKGLPGScdlpqthnlrnkraltllvqmrrlsplsclkdrkdfgfpqekvdaqqikka |
| | fusion | qaipvlseltqqilniftskdssaawnttlldsfcndlhqqlndlqgclmqqvgvqefpltqedallavrkyfhritvylrekkh |
| | protein) | spcawevvraevwralsssanvlgrlreekSGGPGPAGMKGLPGSEVQLVESGGGLVQPGNSLR |
| | | LSCAASGFTFSKFGMSWVRQAPGKGLEWVSSISGSGRDTLYAESVKGRFTISRD |
| | | NAKTTLYLQMNSLRPEDTAVYYCTIGGSLSVSSQGTLVTVSSHHHHHHEPEA |
| 87 | ACP32 | EVQLVESGGGLVQPGNSLRLSCAASGFTFSKFGMSWVRQAPGKGLEWVSSISGS |
| | (mouse | GRDTLYAESVKGRFTISRDNAKTTLYLQMNSLRPEDTAVYYCTIGGSLSVSSQGT |
| | IFNa1 | LVTVSSSGGPGPAGMKGLPGScdlpqthnlrnkraltllvqmttlsplsclkdrkdfgfpqekvdaqqikka |
| | fusion | qaipvlseltqqilniftskdssaawnttlldsfcndlhqqlndlqgclmqqvgvqefpltqedallavrkyfhritvylrekkh |
| | protein) | spcawevvraevwralsssanvSGGPGPAGMKGLPGSEVQLVESGGGLVQPGNSLRLSCA |
| | | ASGFTFSKFGMSWVRQAPGKGLEWVSSISGSGRDTLYAESVKGRFTISRDNAKT |
| | | TLYLQMNSLRPEDTAVYYCTIGGSLSVSSQGTLVTVSSHHHHHHEPEA |
| 88 | IFNgR1 | 10 20 30 40 50 |
| | | MALLFLLPLV MQGVSRAEMG TADLGPSSVP TPINVTIESY NMNPIVYWEY |
| | | 60 70 80 90 100 |
| | | QIMPQVPVFT VEVKNYGVKN SEWIDACINI SHHYCNISDH VGDPSNSLWV |
| | | 110 120 130 140 150 |
| | | RVKARVGQKE SAYAKSEEFA YCRDGKIGPP KLDIRKEEKQ IMIDIFHPSV |
| | | 160 170 180 190 200 |
| | | FVNGDEQEVD YDPEITCYIR VYNVYVRMNG SEIQYKILTQ KEDDCDEIQC |
| | | 210 220 230 240 250 |
| | | QLAIPVSSLN SQYCVSAEGV LHVWGVTTEK SKEVCITIFN SSIKGSLWIP |
| | | 260 270 280 290 300 |
| | | VVAALLLFLV LSLVFICFYI EKINPLKEKS IILPKSLISV VRSATLETKP |
| | | 310 320 330 340 350 |
| | | ESKYVSLITS YQPFSLEKEV VCEEPLSPAT VPGMHIEDNP GKVEHTEELS |
| | | 360 370 380 390 400 |
| | | SIIEVVTIEE NIPDVVPGSH LTPIERESSS PLSSNQSEPG SIALNSYHSR |
| | | 410 420 430 440 450 |
| | | NCSESDHSRN GPDTDSSCLE SHSSLSDSEP PPNNKGEIKT EGQELITVIK |
| | | 460 470 480 |
| | | APTSFGYDKP HVLVDLLVDD SGKESLIGYR PTEDSKEFS |
| 89 | IFNgR2 | 10 20 30 40 50 |
| | | MRPTLLWSLL LLLGVFAAAA AAPPDPLSQL PAPQHPKIRL YNAEQVLSWE |
| | | 60 70 80 90 100 |
| | | PVALSNSTRP VVYQVQFKYT DSKWFIADIM SIGVNCTQIT ATECDETAAS |
| | | 110 120 130 140 150 |
| | | PSAGFPMDFN VTLRLRAELG ALHSAWVTMP WFQHYRNVTV GPPENIEVTP |
| | | 160 170 180 190 200 |
| | | GEGSLIIRFS SPFDIADTST AFFCYYVHYW EKGGIQQVKG PFRSNSISLD |
| | | 210 220 230 240 250 |
| | | NLKPSRVYCL QVQAQLLNNK SNIFRVGHLS NISCYETMAD ASTELQQVIT |
| | | 260 270 280 290 300 |
| | | ISVGTFSLLS WLAGACFFLV LKYRGLIKYW FHTPPSIPLQ IEEYLKDPTQ |
| | | 310 320 330 |
| | | PILEALDKDS SPKDDVWDSV SIISFPEKEQ EDVLQTL |
| 90 | ACP51 | QVQLQESGGGLVQAGGSLRLSCAASGRIFSIDIMSWYRQAPGKQRELVARITRG |
| | Mouse | GTISYDDSVKGRFTISRDNAKNTVYLQMNSLKPEDTGVYYCNALYGTDYWGKG |
| | IFG | TQVTVSSggggsggggsggggsEVQLVESGGGLVQPGNSLRLSCAASGFTFSKFGMSW |
| | fusion | VRQAPGKGLEWVSSISGSGRDTLYAESVKGRFTISRDNAKTTLYLQMNSLRPED |
| | protein | TAVYYCTIGGSLSVSSQGTLVTVSSSGGPGPAGMKGLPGShgtviesleslnnyfnssgidve |
| | | ekslfldiwrnwqkdgdmkilqsqiisfylrlfevlkdnqaisnnisvieshlittffsnskakkdafmsiakfevnnpqvqr |
| | | qafnelirvvhqllpesslrkrkrsrcSGGPGPAGMKGLPGSEVQLVESGGGLVQPGNSLRLSC |
| | | AASGFTFSKFGMSWVRQAPGKGLEWVSSISGSGRDTLYAESVKGRFTISRDNAK |
| | | TTLYLQMNSLRPEDTAVYYCTIGGSLSVSSQGTLVTVSSHHHHHH |
| 91 | ACP52 | EVQLVESGGGLVQPGNSLRLSCAASGFTFSKFGMSWVRQAPGKGLEWVSSISGS |
| | Mouse | GRDTLYAESVKGRFTISRDNAKTTLYLQMNSLRPEDTAVYYCTIGGSLSVSSQGT |
| | IFG | LVTVSSSGGPGPAGMKGLPGShgtviesleslnnyfnssgidveekslfldiwrnwqkdgdmkilqsqiisf |
| | fusion | ylrlfevlkdnqaisnnisvieshlittffsnskakkdafmsiakfevnnpqvqrqafnelirvvhqllpesslrkrkrsrcSG |
| | protein | GPGPAGMKGLPGSEVQLVESGGGLVQPGNSLRLSCAASGFTFSKFGMSWVRQA |
| | | PGKGLEWVSSISGSGRDTLYAESVKGRFTISRDNAKTTLYLQMNSLRPEDTAVY |
| | | YCTIGGSLSVSSQGTLVTVSSSGGPGPAGMKGLPGSEVQLVESGGGLVQPGNSL |
| | | RLSCAASGFTFSKFGMSWVRQAPGKGLEWVSSISGSGRDTLYAESVKGRFTISR |
| | | DNAKTTLYLQMNSLRPEDTAVYYCTIGGSLSVSSQGTLVTVSSggggsggggsggggs |
| | | QVQLQESGGGLVQAGGSLRLSCAASGRIFSIDIMSWYRQAPGKQRELVARITRG |
| | | GTISYDDSVKGRFTISRDNAKNTVYLQMNSLKPEDTGVYYCNALYGTDYWGKG |
| | | TQVTVSSHHHHHH |
| 92 | ACP53 | eahkseiahryndlgeqhfkglvliafsqylqkcsydehaklvqevtdfaktcvadesaancdkslhtlfgdklcaipnlren |
| | Mouse | ygeladcctkqepemecflqhkddnpslppferpeaeamctsfkenpttfmghylhevarrhpyfyapellyyaeqynei |
| | IFG | ltqccaeadkescltpkldgvkekalvssvrqrmkcssmqkfgerafkawavarlsqtfpnadfaeitklatdltkvnkecc |
| | fusion | hgdllecaddraelakymcenqatissklqtccdkpllkkahclsevehdtmpadlpaiaadfvedqevcknyaeakdvfl |
| | protein | gtflyeysrrhpdysyslllrlakkyeatlekccaeanppacygtvlaefqplveepknlvktncdlyeklgeygfqnailvry |
| | | tqkapqvstptiveaarnlgrvgtkcctlpedqrlpcvedylsailnrvcllhektpvsehvtkccsgslverrpcfsaltvdety |
| | | vpkefkaetftfhsdictlpekekqikkqtalaelvkhkpkataeqlktvmddfaqfldtcckaadkdtcfstegpnlvtrckd |
| | | alaSGGPGPAGMKGLPGShgtviesleslnnyfnssgidveekslfldiwrnwqkdgdmkilqsqiisfylrlfev |
| | | lkdnqaisnnisvieshlittffsnskakkdafmsiakfevnnpqvqrqafnelirvvhqllpesslrkrkrsrcSGGPGPA |
| | | GMKGLPGSeahkseiahryndlgeqhfkglvliafsqylqkcsydehaklvqevtdfaktcvadesaancdkslhtlf |
| | | gdklcaipnlrenygeladcctkqepernecflqhkddnpslppferpeaeamctsfkenpttfmghylhevarrhpyfya |
| | | pellyyaeqyneiltqccaeadkescltpkldgvkekalvssvrqrmkcssmqkfgerafkawavarlsqtfpnadfaeitk |
| | | latdltkvnkecchgdllecaddraelakymcenqatissklqtccdkpllkkahclsevehdtmpadlpaiaadfvedqev |
| | | cknyaeakdvflgtflyeysrrhpdysyslllrlakkyeatlekccaeanppacygtvlaefqplveepknlvktncdlyekl |
| | | geygfqnailvrytqkapqvstptlveaarnlgrvgtkcctlpedqrlpcvedylsailnrycllhektpvsehvtkccsgslve |
| | | rrpcfsaltvdetyvpkefkaetftfhsdictlpekekqikkqtalaelvkhkpkataeqlktvmddfaqfldtcckaadkdtc |
| | | fstegpnlvtrckdalaHHHHHH |
| 93 | ACP54 | eahkseiahryndlgeqhfkglvliafsqylqkcsydehaklvqevtdfaktcvadesaancdkslhtlfgdklcaipnlren |
| | Mouse | ygeladcctkqepemecflqhkddnpslppferpeaeamctsfkenpttfmghylhevarrhpyfyapellyyaeqynei |
| | IFG | ltqccaeadkescltpkldgvkekalvssvrqrmkcssmqkfgerafkawavarlsqtfpnadfaeitklatdltkvnkecc |
| | fusion | hgdllecaddraelakymcenqatissklqtccdkpllkkahclsevehdtmpadlpaiaadfvedqevcknyaeakdvfl |
| | protein | gtflyeysrrhpdysvslllrlakkyeatlekccaeanppacygtvlaefqplveepknlvktncdlyeklgeygfqnailvry |
| | | tqkapqvstptlveaarnlgrvgtkcctlpedqrlpcvedylsailnrvcllhektpvsehvtkccsgslverrpcfsaltvdety |
| | | vpkefkaetftfhsdictlpekekqikkqtalaelvkhkpkataeqlktvmddfaqfldtcckaadkdtcfstegpnlvtrckd |
| | | alaSGGPGPAGMKGLPGShgtviesleslnnyfnssgidveekslfldiwrnwqkdgdmkilqsqiisfylrlfev |
| | | lkdnqaisnnisvieshlittffsnskakkdafmsiakfevnnpqvqrqafnelirvvhqllpesslrkrkrsrcggggsgggg |
| | | sggggshgtviesleslnnyfnssgidveekslfldiwrnwqkdgdmkilqsqiisfylrlfevlkdnqaisnnisvieshlitt |
| | | ffsnskakkdafmsiakfevnnpqvqrqafnelirvvhqllpesslrkrkrsrcSGGPGPAGMKGLPGSeahkse |
| | | iahryndlgeqhfkglvliafsqylqkcsydehaklvqevtdfaktcvadesaancdkslhtlfgdklcaipnlrenygelad |
| | | cctkqepernecflqhkddnpslppferpeaeamctsfkenpttfmghylhevarrhpyfyapellyyaeqyneiltqccae |
| | | adkescltpkldgvkekalvssvrqrmkcssmqkfgerafkawavarlsqtfpnadfaeitklatdltkvnkecchgdllec |
| | | addraelakymcenqatissklqtccdkpllkkahclsevehdtmpadlpaiaadfvedqevcknyaeakdvflgtflyey |
| | | srrhpdysyslllrlakkyeatlekccaeanppacygtvlaefqplveepknlvktncdlyeklgeygfqnailvrytqkapq |
| | | vstptlveaarnlgrvgtkcctlpedqrlpcvedylsailnrycllhektpvsehvtkccsgslverrpcfsaltvdetyvpkefk |
| | | aetftfhsdictlpekekqikkqtalaelvkhkpkataeqlktvmddfaqfldtcckaadkdtcfstegpnlvtrckdalaHH |
| | | HHHH |
| 94 | ACP50 | mdmrvpaqllgllllwlrgarcQVQLQESGGGLVQAGGSLRLSCAASGRIFSIDIMSWYRQA |
| | Mouse | PGKQRELVARITRGGTISYDDSVKGRFTISRDNAKNTVYLQMNSLKPEDTGVYY |
| | IFG | CNALYGTDYWGKGTQVTVSSggggsggggsggggsEVQLVESGGGLVQPGNSLRLSC |
| | fusion | AASGFTFSKFGMSWVRQAPGKGLEWVSSISGSGRDTLYAESVKGRFTISRDNAK |
| | protein | TTLYLQMNSLRPEDTAVYYCTIGGSLSVSSQGTLVTVSSSGGPGPAGMKGLPGSh |
| | | gtviesleslnnyfnssgidveekslfldiwrnwqkdgdmkilqsqiisfylrlfevlkdnqaisnnisvieshlittffsnskak |
| | | kdafmsiakfevnnpqvqrqafnelirvvhqllpesslrkrkrsrcggggsggggsggggshgtviesleslnnyfnssgid |
| | | veekslfldiwrnwqkdgdmkilqsqiisfylrlfevlkdnqaisnnisvieshlittffsnskakkdafmsiakfevnnpqv |
| | | qrqafnelirvvhqllpesslrkrkrsrcSGGPGPAGMKGLPGSEVQLVESGGGLVQPGNSLRLS |
| | | CAASGFTFSKFGMSWVRQAPGKGLEWVSSISGSGRDTLYAESVKGRFTISRDNA |
| | | KTTLYLQMNSLRPEDTAVYYCTIGGSLSVSSQGTLVTVSSHHHHHH |
| 95 | ACP55 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFG | YYCTIGGSLSVSSQGTLVTVSSSGGPGPAGMKGLPGShgtviesleslnnyfnssgidveekslfl |
| | fusion | diwrnwqkdgdmkilqsqiisfylrlfevlkdnqaisnnisvieshlittffsnskakkdafmsiakfevnnpqvqrqafnel |
| | protein | irvvhqllpesslrkrkrsrcSGGPGPAGMKGLPGSEVQLVESGGGLVQPGNSLRLSCAASG |
| | | FTFSKFGMSWVRQAPGKGLEWVSSISGSGRDTLYAESVKGRFTISRDNAKTTLY |
| | | LQMNSLRPEDTAVYYCTIGGSLSVSSQGTLVTVSSSGGPGPAGMKGLPGShgtvies |
| | | leslnnyfnssgidveekslfldiwrnwqkdgdmkilqsqiisfylrlfevlkdnqaisnnisvieshlittffsnskakkdaf |
| | | msiakfevnnpqvqrqafnelirvvhqllpesslrkrkrsrcSGGPGPAGMKGLPGSEVQLVESGGGL |
| | | VQPGNSLRLSCAASGFTFSKFGMSWVRQAPGKGLEWVSSISGSGRDTLYAESVK |
| | | GRFTISRDNAKTTLYLQMNSLRPEDTAVYYCTIGGSLSVSSQGTLVTVSSHHHHH |
| | | H |
| 96 | ACP56 | mdmrvpaqllgllllwlrgarcQVQLQESGGGLAQAGGSLSLSCAASGFTVSNSVMAWYR |
| | Mouse | QTPGKQREFVAIINSVGSTNYADSVKGRFTISRDNAKNTVYLQMNNLKPEDTAV |
| | IFG | YVCNRNFDRIYWGQGTQVTVSSggggsggggsggggsEVQLVESGGGLVQPGNSLRLS |
| | fusion | CAASGFTFSKFGMSWVRQAPGKGLEWVSSISGSGRDTLYAESVKGRFTISRDNA |
| | protein | KTTLYLQMNSLRPEDTAVYYCTIGGSLSVSSQGTLVTVSSSGGPGPAGMKGLPG |
| | | Shgtviesleslnnyfnssgidveekslfldiwrnwqkdgdmkilqsqiisfylrlfevlkdnqaisnnisvieshlittffsnsk |
| | | akkdafmsiakfevnnpqvqrqafnelirvvhqllpesslrkrkrsrcSGGPGPAGMKGLPGSEVQLVES |
| | | GGGLVQPGNSLRLSCAASGFTFSKFGMSWVRQAPGKGLEWVSSISGSGRDTLYA |
| | | ESVKGRFTISRDNAKTTLYLQMNSLRPEDTAVYYCTIGGSLSVSSQGTLVTVSSH |
| | | HHHHHEPEA |
| 97 | ACP57 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFG | YYCTIGGSLSVSSQGTLVTVSSSGGPGPAGMKGLPGShgtviesleslnnyfnssgidveekslfl |
| | fusion | diwrnwqkdgdmkilqsqiisfylrlfevlkdnqaisnnisvieshlittffsnskakkdafmsiakfevnnpqvqrqafnel |
| | protein | irvvhqllpesslrkrkrsrcSGGPGPAGMKGLPGSEVQLVESGGGLVQPGNSLRLSCAASG |
| | | FTFSKFGMSWVRQAPGKGLEWVSSISGSGRDTLYAESVKGRFTISRDNAKTTLY |
| | | LQMNSLRPEDTAVYYCTIGGSLSVSSQGTLVTVSSggggsggggsggggsQVQLQESG |
| | | GGLAQAGGSLSLSCAASGFTVSNSVMAWYRQTPGKQREFVAIINSVGSTNYADS |
| | | VKGRFTISRDNAKNTVYLQMNNLKPEDTAVYVCNRNFDRIYWGQGTQVTVSSH |
| | | HHHHHEPEA |
| 98 | ACP58 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFG | YYCTIGGSLSVSSQGTLVTVSSSGGPGPAGMKGLPGShgtviesleslnnyfnssgidveekslfl |
| | fusion | diwrnwqkdgdmkilqsqiisfylrlfevlkdnqaisnnisvieshlittffsnskakkdafmsiakfevnnpqvqrqafnel |
| | protein | irvvhqllpesslrkrkrsrcSGGPGPAGMKGLPGShgtviesleslnnyfnssgidveekslfldiwrnwqkdgd |
| | | mkilqsqiisfylrlfevlkdnqaisnnisvieshlittffsnskakkdafmsiakfevnnpqvqrqafnelirvvhqllpesslr |
| | | krkrsrcSGGPGPAGMKGLPGSEVQLVESGGGLVQPGNSLRLSCAASGFTFSKFGMS |
| | | WVRQAPGKGLEWVSSISGSGRDTLYAESVKGRFTISRDNAKTTLYLQMNSLRPE |
| | | DTAVYYCTIGGSLSVSSQGTLVTVSSggggsggggsggggsQVQLQESGGGLVQAGGS |
| | | LRLSCAASGRIFSIDIMSWYRQAPGKQRELVARITRGGTISYDDSVKGRFTISRDN |
| | | AKNTVYLQMNSLKPEDTGVYYCNALYGTDYWGKGTQVTVSSHHHHHHEPEA |
| 99 | ACP59 | mdmrvpaqllgllllwlrgarcQVQLQESGGGLAQAGGSLSLSCAASGFTVSNSVMAWYR |
| | Mouse | QTPGKQREFVAIINSVGSTNYADSVKGRFTISRDNAKNTVYLQMNNLKPEDTAV |
| | IFG | YVCNRNFDRIYWGQGTQVTVSSggggsggggsggggsEVQLVESGGGLVQPGNSLRLS |
| | fusion | CAASGFTFSKFGMSWVRQAPGKGLEWVSSISGSGRDTLYAESVKGRFTISRDNA |
| | protein | KTTLYLQMNSLRPEDTAVYYCTIGGSLSVSSQGTLVTVSSSGGPGPAGMKGLPG |
| | | Shgtviesleslnnyfnssgidveekslfldiwrnwqkdgdmkilqsqiisfylrlfevlkdnqaisnnisvieshlittffsnsk |
| | | akkdafmsiakfevnnpqvqrqafnelirvvhqllpesslrkrkrsrcSGGPGPAGMKGLPGShgtvieslesln |
| | | nyfnssgidveekslfldiwrnwqkdgdmkilqsqiisfylrlfevlkdnqaisnnisvieshlittffsnskakkdafmsiak |
| | | fevnnpqvqrqafnelirvvhqllpesslrkrkrsrcSGGPGPAGMKGLPGSEVQLVESGGGLVQPG |
| | | NSLRLSCAASGFTFSKFGMSWVRQAPGKGLEWVSSISGSGRDTLYAESVKGRFTI |
| | | SRDNAKTTLYLQMNSLRPEDTAVYYCTIGGSLSVSSQGTLVTVSSHHHHHHEPE |
| | | A |
| 100 | ACP60 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFG | YYCTIGGSLSVSSQGTLVTVSSSGGPGPAGMKGLPGShgtviesleslnnyfnssgidveekslfl |
| | fusion | diwrnwqkdgdmkilqsqiisfylrlfevlkdnqaisnnisvieshlittffsnskakkdafmsiakfevnnpqvqrqafnel |
| | protein | irvvhqllpesslrkrkrsrcSGGPGPAGMKGLPGShgtviesleslnnyfnssgidveekslfldiwrnwqkdgd |
| | | mkilqsqiisfylrlfevlkdnqaisnnisvieshlittffsnskakkdafmsiakfevnnpqvqrqafnelirvvhqllpesslr |
| | | krkrsrcSGGPGPAGMKGLPGSEVQLVESGGGLVQPGNSLRLSCAASGFTFSKFGMS |
| | | WVRQAPGKGLEWVSSISGSGRDTLYAESVKGRFTISRDNAKTTLYLQMNSLRPE |
| | | DTAVYYCTIGGSLSVSSQGTLVTVSSggggsggggsggggsQVQLQESGGGLAQAGGS |
| | | LSLSCAASGFTVSNSVMAWYRQTPGKQREFVAIINSVGSTNYADSVKGRFTISRD |
| | | NAKNTVYLQMNNLKPEDTAVYVCNRNFDRIYWGQGTQVTVSSHHHHHHEPEA |
| 101 | ACP61 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFG | YYCTIGGSLSVSSQGTLVTVSSSGGPGPAGMKGLPGShgtviesleslnnyfnssgidveekslfl |
| | fusion | diwrnwqkdgdmkilqsqiisfylrlfevlkdnqaisnnisvieshlittffsnskakkdafmsiakfevnnpqvqrqafnel |
| | protein | irvvhqllpesslrkrkrsrcSGGPGPAGMKGLPGShgtviesleslnnyfnssgidveekslfldiwrnwqkdgd |
| | | mkilqsqiisfylrlfevlkdnqaisnnisvieshlittffsnskakkdafmsiakfevnnpqvqrqafnelirvvhqllpesslr |
| | | krkrsrcSGGPGPAGMKGLPGSEVQLVESGGGLVQPGNSLRLSCAASGFTFSKFGMS |
| | | WVRQAPGKGLEWVSSISGSGRDTLYAESVKGRFTISRDNAKTTLYLQMNSLRPE |
| | | DTAVYYCTIGGSLSVSSQGTLVTVSSggggsggggsggggsEVQLVESGGGLVQPGGSL |
| | | RLSCAASGFTFSSYAMSWVRQAPGKGLEWVSAISGSGGSTYYADSVKGRFTISR |
| | | DNSKNTLYLQMNSLRAEDTAVYYCARGVGAFRPYRKHEWGQGTLVTVSRgggg |
| | | sggggsggggsSSELTQDPAVSVALGQTVRITCQGDSLRSYYASWYQQKPGQAPVLV |
| | | IYGKNNRPSGIPDRFSGSSSGNTASLTTTGAQAEDEADYYCNSSPFEHNLVVFGG |
| | | GTKLTVLHHHHHHEPEA |
| 102 | ACP63 | mdmrvpaqllgllllwlrgarcEVQLVESGGGLVQPGGSLRLSCAASGFTFSSYAMSWVRQ |
| | Anti-FN | APGKGLEWVSAISGSGGSTYYADSVKGRFTISRDNSKNTLYLQMNSLRAEDTAV |
| | CGS-2 | YYCARGVGAFRPYRKHEWGQGTLVTVSRggggsggggsggggsSSELTQDPAVSVAL |
| | scFv | GQTVRITCQGDSLRSYYASWYQQKPGQAPVLVIYGKNNRPSGIPDRFSGSSSGNT |
| | | ASLTTTGAQAEDEADYYCNSSPFEHNLVVFGGGTKLTVLHHHHHHEPEA |
| 103 | ACP69 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFG | YYCTIGGSLSVSSQGTLVTVSSSGGPGPAGMKGLPGShgtviesleslnnyfnssgidveekslfl |
| | fusion | diwrnwqkdgdmkilqsqiisfylrlfevlkdnqaisnnisvieshlittffsnskakkdafmsiakfevnnpqvqrqafnel |
| | protein | irvvhqllpesslrkrkrsrcSGGPGPAGMKGLPGSEVQLVESGGGLVQPGNSLRLSCAASG |
| | | FTFSKFGMSWVRQAPGKGLEWVSSISGSGRDTLYAESVKGRFTISRDNAKTTLY |
| | | LQMNSLRPEDTAVYYCTIGGSLSVSSQGTLVTVSSSGGPGPAGMKGLPGShgtvies |
| | | leslnnyfnssgidveekslfldiwrnwqkdgdmkilqsqiisfylrlfevlkdnqaisnnisvieshlittffsnskakkdaf |
| | | msiakfevnnpqvqrqafnelirvvhqllpesslrkrkrsrcHHHHHHEPEA |
| 104 | ACP70 | mdmrvpaqllgllllwlrgarchgtviesleslnnyfnssgidveekslfldiwrnwqkdgdmkilqsqiisfylrlfevlkd |
| | Mouse | nqaisnnisvieshlittffsnskakkdafmsiakfevnnpqvqrqafnelirvvhqllpesslrkrkrsrcSGGPGPAG |
| | IFG | MKGLPGSEVQLVESGGGLVQPGNSLRLSCAASGFTFSKFGMSWVRQAPGKGLE |
| | fusion | WVSSISGSGRDTLYAESVKGRFTISRDNAKTTLYLQMNSLRPEDTAVYYCTIGGS |
| | protein | LSVSSQGTLVTVSSSGGPGPAGMKGLPGShgtviesleslnnyfnssgidveekslfldiwrnwqkdg |
| | | dmkilqsqiisfylrlfevlkdnqaisnnisvieshlittffsnskakkdafmsiakfevnnpqvqrqafnelirvvhqllpessl |
| | | rkrkrsrcSGGPGPAGMKGLPGSEVQLVESGGGLVQPGNSLRLSCAASGFTFSKFGM |
| | | SWVRQAPGKGLEWVSSISGSGRDTLYAESVKGRFTISRDNAKTTLYLQMNSLRP |
| | | EDTAVYYCTIGGSLSVSSQGTLVTVSSHHHHHHEPEA |
| 105 | ACP71 | mdmrvpaqllgllllwlrgarchgtviesleslnnyfnssgidveekslfldiwrnwqkdgdmkilqsqiisfylrlfevlkd |
| | Mouse | nqaisnnisvieshlittffsnskakkdafmsiakfevnnpqvqrqafnelirvvhqllpesslrkrkrsrcSGGPGPAG |
| | IFG | MKGLPGSEAHKSEIAHRYNDLGEQHFKGLVLIAFSQYLQKCSYDEHAKLVQEVT |
| | fusion | DFAKTCVADESAANCDKSLHTLFGDKLCAIPNLRENYGELADCCTKQEPERNEC |
| | protein | FLQHKDDNPSLPPFERPEAEAMCTSFKENPTTFMGHYLHEVARRHPYFYAPELL |
| | | YYAEQYNEILTQCCAEADKESCLTPKLDGVKEKALVSSVRQRMKCSSMQKFGE |
| | | RAFKAWAVARLSQTFPNADFAEITKLATDLTKVNKECCHGDLLECADDRAELA |
| | | KYMCENQATISSKLQTCCDKPLLKKAHCLSEVEHDTMPADLPAIAADFVEDQEV |
| | | CKNYAEAKDVFLGTFLYEYSRRHPDYSVSLLLRLAKKYEATLEKCCAEANPPAC |
| | | YGTVLAEFQPLVEEPKNLVKTNCDLYEKLGEYGFQNAILVRYTQKAPQVSTPTL |
| | | VEAARNLGRVGTKCCTLPEDQRLPCVEDYLSAILNRVCLLHEKTPVSEHVTKCC |
| | | SGSLVERRPCFSALTVDETYVPKEFKAETFTFHSDICTLPEKEKQIKKQTALAELV |
| | | KHKPKATAEQLKTVMDDFAQFLDTCCKAADKDTCFSTEGPNLVTRCKDALASG |
| | | GPGPAGMKGLPGShgtviesleslnnyfnssgidveekslfldiwrnwqkdgdmkilqsqiisfylrlfevlkdnq |
| | | aisnnisvieshlittffsnskakkdafmsiakfevnnpqvqrqafnelirvvhqllpesslrkrkrsrcSGGPGPAGM |
| | | KGLPGSEAHKSEIAHRYNDLGEQHFKGLVLIAFSQYLQKCSYDEHAKLVQEVTD |
| | | FAKTCVADESAANCDKSLHTLFGDKLCAIPNLRENYGELADCCTKQEPERNECF |
| | | LQHKDDNPSLPPFERPEAEAMCTSFKENPTTFMGHYLHEVARRHPYFYAPELLY |
| | | YAEQYNEILTQCCAEADKESCLTPKLDGVKEKALVSSVRQRMKCSSMQKFGER |
| | | AFKAWAVARLSQTFPNADFAEITKLATDLTKVNKECCHGDLLECADDRAELAK |
| | | YMCENQATISSKLQTCCDKPLLKKAHCLSEVEHDTMPADLPAIAADFVEDQEVC |
| | | KNYAEAKDVFLGTFLYEYSRRHPDYSVSLLLRLAKKYEATLEKCCAEANPPACY |
| | | GTVLAEFQPLVEEPKNLVKTNCDLYEKLGEYGFQNAILVRYTQKAPQVSTPTLV |
| | | EAARNLGRVGTKCCTLPEDQRLPCVEDYLSAILNRVCLLHEKTPVSEHVTKCCS |
| | | GSLVERRPCFSALTVDETYVPKEFKAETFTFHSDICTLPEKEKQIKKQTALAELVK |
| | | HKPKATAEQLKTVMDDFAQFLDTCCKAADKDTCFSTEGPNLVTRCKDALAHHH |
| | | HHHEPEA |
| 106 | ACP72 | mdmrvpaqllgllllwlrgarcEAHKSEIAHRYNDLGEQHFKGLVLIAFSQYLQKCSYDEHA |
| | Mouse | KLVQEVTDFAKTCVADESAANCDKSLHTLFGDKLCAIPNLRENYGELADCCTK |
| | IFG | QEPERNECFLQHKDDNPSLPPFERPEAEAMCTSFKENPTTFMGHYLHEVARRHP |
| | fusion | YFYAPELLYYAEQYNEILTQCCAEADKESCLTPKLDGVKEKALVSSVRQRMKCS |
| | protein | SMQKFGERAFKAWAVARLSQTFPNADFAEITKLATDLTKVNKECCHGDLLECA |
| | | DDRAELAKYMCENQATISSKLQTCCDKPLLKKAHCLSEVEHDTMPADLPAIAAD |
| | | FVEDQEVCKNYAEAKDVFLGTFLYEYSRRHPDYSVSLLLRLAKKYEATLEKCCA |
| | | EANPPACYGTVLAEFQPLVEEPKNLVKTNCDLYEKLGEYGFQNAILVRYTQKAP |
| | | QVSTPTLVEAARNLGRVGTKCCTLPEDQRLPCVEDYLSAILNRVCLLHEKTPVSE |
| | | HVTKCCSGSLVERRPCFSALTVDETYVPKEFKAETFTFHSDICTLPEKEKQIKKQT |
| | | ALAELVKHKPKATAEQLKTVMDDFAQFLDTCCKAADKDTCFSTEGPNLVTRCK |
| | | DALASGGPGPAGMKGLPGShgtviesleslnnyfnssgidveekslfldiwrnwqkdgdmkilqsqiisfylr |
| | | lfevlkdnqaisnnisvieshlittffsnskakkdafmsiakfevnnpqvqrqafnelirvvhqllpesslrkrkrsrcSGGP |
| | | GPAGMKGLPGSEAHKSEIAHRYNDLGEQHFKGLVLIAFSQYLQKCSYDEHAKLV |
| | | QEVTDFAKTCVADESAANCDKSLHTLFGDKLCAIPNLRENYGELADCCTKQEPE |
| | | RNECFLQHKDDNPSLPPFERPEAEAMCTSFKENPTTFMGHYLHEVARRHPYFYA |
| | | PELLYYAEQYNEILTQCCAEADKESCLTPKLDGVKEKALVSSVRQRMKCSSMQ |
| | | KFGERAFKAWAVARLSQTFPNADFAEITKLATDLTKVNKECCHGDLLECADDR |
| | | AELAKYMCENQATISSKLQTCCDKPLLKKAHCLSEVEHDTMPADLPAIAADFVE |
| | | DQEVCKNYAEAKDVFLGTFLYEYSRRHPDYSVSLLLRLAKKYEATLEKCCAEA |
| | | NPPACYGTVLAEFQPLVEEPKNLVKTNCDLYEKLGEYGFQNAILVRYTQKAPQV |
| | | STPTLVEAARNLGRVGTKCCTLPEDQRLPCVEDYLSAILNRVCLLHEKTPVSEHV |
| | | TKCCSGSLVERRPCFSALTVDETYVPKEFKAETFTFHSDICTLPEKEKQIKKQTAL |
| | | AELVKHKPKATAEQLKTVMDDFAQFLDTCCKAADKDTCFSTEGPNLVTRCKDA |
| | | LASGGPGPAGMKGLPGShgtviesleslnnyfnssgidveekslfldiwrnwqkdgdmkilqsqiisfylrlfe |
| | | vlkdnqaisnnisvieshlittffsnskakkdafmsiakfevnnpqvqrqafnelirvvhqllpesslrkrkrsrcHHHHH |
| | | HEPEA |
| 107 | ACP73 | mdmrvpaqllgllllwlrgarcEAHKSEIAHRYNDLGEQHFKGLVLIAFSQYLQKCSYDEHA |
| | Mouse | KLVQEVTDFAKTCVADESAANCDKSLHTLFGDKLCAIPNLRENYGELADCCTK |
| | IFG | QEPERNECFLQHKDDNPSLPPFERPEAEAMCTSFKENPTTFMGHYLHEVARRHP |
| | fusion | YFYAPELLYYAEQYNEILTQCCAEADKESCLTPKLDGVKEKALVSSVRQRMKCS |
| | protein | SMQKFGERAFKAWAVARLSQTFPNADFAEITKLATDLTKVNKECCHGDLLECA |
| | | DDRAELAKYMCENQATISSKLQTCCDKPLLKKAHCLSEVEHDTMPADLPAIAAD |
| | | FVEDQEVCKNYAEAKDVFLGTFLYEYSRRHPDYSVSLLLRLAKKYEATLEKCCA |
| | | EANPPACYGTVLAEFQPLVEEPKNLVKTNCDLYEKLGEYGFQNAILVRYTQKAP |
| | | QVSTPTLVEAARNLGRVGTKCCTLPEDQRLPCVEDYLSAILNRVCLLHEKTPVSE |
| | | HVTKCCSGSLVERRPCFSALTVDETYVPKEFKAETFTFHSDICTLPEKEKQIKKQT |
| | | ALAELVKHKPKATAEQLKTVMDDFAQFLDTCCKAADKDTCFSTEGPNLVTRCK |
| | | DALASGGPGPAGMKGLPGShgtviesleslnnyfnssgidveekslfldiwrnwqkdgdmkilqsqiisfylr |
| | | lfevlkdnqaisnnisvieshlittffsnskakkdafmsiakfevnnpqvqrqafnelirvvhqllpesslrkrkrsrcSGGP |
| | | GPAGMKGLPGSEAHKSEIAHRYNDLGEQHFKGLVLIAFSQYLQKCSYDEHAKLV |
| | | QEVTDFAKTCVADESAANCDKSLHTLFGDKLCAIPNLRENYGELADCCTKQEPE |
| | | RNECFLQHKDDNPSLPPFERPEAEAMCTSFKENPTTFMGHYLHEVARRHPYFYA |
| | | PELLYYAEQYNEILTQCCAEADKESCLTPKLDGVKEKALVSSVRQRMKCSSMQ |
| | | KFGERAFKAWAVARLSQTFPNADFAEITKLATDLTKVNKECCHGDLLECADDR |
| | | AELAKYMCENQATISSKLQTCCDKPLLKKAHCLSEVEHDTMPADLPAIAADFVE |
| | | DQEVCKNYAEAKDVFLGTFLYEYSRRHPDYSVSLLLRLAKKYEATLEKCCAEA |
| | | NPPACYGTVLAEFQPLVEEPKNLVKTNCDLYEKLGEYGFQNAILVRYTQKAPQV |
| | | STPTLVEAARNLGRVGTKCCTLPEDQRLPCVEDYLSAILNRVCLLHEKTPVSEHV |
| | | TKCCSGSLVERRPCFSALTVDETYVPKEFKAETFTFHSDICTLPEKEKQIKKQTAL |
| | | AELVKHKPKATAEQLKTVMDDFAQFLDTCCKAADKDTCFSTEGPNLVTRCKDA |
| | | LASGGPGPAGMKGLPGShgtviesleslnnyfnssgidveekslfldiwrnwqkdgdmkilqsqiisfylrlfe |
| | | vlkdnqaisnnisvieshlittffsnskakkdafmsiakfevnnpqvqrqafnelirvvhqllpesslrkrkrsrcSGGPGP |
| | | AGMKGLPGSEAHKSEIAHRYNDLGEQHFKGLVLIAFSQYLQKCSYDEHAKLVQ |
| | | EVTDFAKTCVADESAANCDKSLHTLFGDKLCAIPNLRENYGELADCCTKQEPER |
| | | NECFLQHKDDNPSLPPFERPEAEAMCTSFKENPTTFMGHYLHEVARRHPYFYAP |
| | | ELLYYAEQYNEILTQCCAEADKESCLTPKLDGVKEKALVSSVRQRMKCSSMQK |
| | | FGERAFKAWAVARLSQTFPNADFAEITKLATDLTKVNKECCHGDLLECADDRA |
| | | ELAKYMCENQATISSKLQTCCDKPLLKKAHCLSEVEHDTMPADLPAIAADFVED |
| | | QEVCKNYAEAKDVFLGTFLYEYSRRHPDYSVSLLLRLAKKYEATLEKCCAEANP |
| | | PACYGTVLAEFQPLVEEPKNLVKTNCDLYEKLGEYGFQNAILVRYTQKAPQVST |
| | | PTLVEAARNLGRVGTKCCTLPEDQRLPCVEDYLSAILNRVCLLHEKTPVSEHVT |
| | | KCCSGSLVERRPCFSALTVDETYVPKEFKAETFTFHSDICTLPEKEKQIKKQTALA |
| | | ELVKHKPKATAEQLKTVMDDFAQFLDTCCKAADKDTCFSTEGPNLVTRCKDAL |
| | | AHHHHHHEPEA |
| 108 | ACP74 | mdmrvpaqllgllllwlrgarcEAHKSEIAHRYNDLGEQHFKGLVLIAFSQYLQKCSYDEHA |
| | Mouse | KLVQEVTDFAKTCVADESAANCDKSLHTLFGDKLCAIPNLRENYGELADCCTK |
| | IFG | QEPERNECFLQHKDDNPSLPPFERPEAEAMCTSFKENPTTFMGHYLHEVARRHP |
| | fusion | YFYAPELLYYAEQYNEILTQCCAEADKESCLTPKLDGVKEKALVSSVRQRMKCS |
| | protein | SMQKFGERAFKAWAVARLSQTFPNADFAEITKLATDLTKVNKECCHGDLLECA |
| | | DDRAELAKYMCENQATISSKLQTCCDKPLLKKAHCLSEVEHDTMPADLPAIAAD |
| | | FVEDQEVCKNYAEAKDVFLGTFLYEYSRRHPDYSVSLLLRLAKKYEATLEKCCA |
| | | EANPPACYGTVLAEFQPLVEEPKNLVKTNCDLYEKLGEYGFQNAILVRYTQKAP |
| | | QVSTPTLVEAARNLGRVGTKCCTLPEDQRLPCVEDYLSAILNRVCLLHEKTPVSE |
| | | HVTKCCSGSLVERRPCFSALTVDETYVPKEFKAETFTFHSDICTLPEKEKQIKKQT |
| | | ALAELVKHKPKATAEQLKTVMDDFAQFLDTCCKAADKDTCFSTEGPNLVTRCK |
| | | DALASGGPGPAGMKGLPGShgtviesleslnnyfnssgidveekslfldiwrnwqkdgdmkilqsqiisfylr |
| | | lfevlkdnqaisnnisvieshlittffsnskakkdafmsiakfevnnpqvqrqafnelirvvhqllpesslrkrkrsrcSGGP |
| | | GPAGMKGLPGSggggsEAHKSEIAHRYNDLGEQHFKGLVLIAFSQYLQKCSYDEH |
| | | AKLVQEVTDFAKTCVADESAANCDKSLHTLFGDKLCAIPNLRENYGELADCCT |
| | | KQEPERNECFLQHKDDNPSLPPFERPEAEAMCTSFKENPTTFMGHYLHEVARRH |
| | | PYFYAPELLYYAEQYNEILTQCCAEADKESCLTPKLDGVKEKALVSSVRQRMKC |
| | | SSMQKFGERAFKAWAVARLSQTFPNADFAEITKLATDLTKVNKECCHGDLLEC |
| | | ADDRAELAKYMCENQATISSKLQTCCDKPLLKKAHCLSEVEHDTMPADLPAIAA |
| | | DFVEDQEVCKNYAEAKDVFLGTFLYEYSRRHPDYSVSLLLRLAKKYEATLEKCC |
| | | AEANPPACYGTVLAEFQPLVEEPKNLVKTNCDLYEKLGEYGFQNAILVRYTQKA |
| | | PQVSTPTLVEAARNLGRVGTKCCTLPEDQRLPCVEDYLSAILNRVCLLHEKTPVS |
| | | EHVTKCCSGSLVERRPCFSALTVDETYVPKEFKAETFTFHSDICTLPEKEKQIKKQ |
| | | TALAELVKHKPKATAEQLKTVMDDFAQFLDTCCKAADKDTCFSTEGPNLVTRC |
| | | KDALAggggsSGGPGPAGMKGLPGShgtviesleslnnyfnssgidveekslfldiwrnwqkdgdmkilq |
| | | sqiisfylrlfevlkdnqaisnnisvieshlittffsnskakkdafmsiakfevnnpqvqrqafnelirvvhqllpesslrkrkrsr |
| | | cSGGPGPAGMKGLPGSEAHKSEIAHRYNDLGEQHFKGLVLIAFSQYLQKCSYDE |
| | | HAKLVQEVTDFAKTCVADESAANCDKSLHTLFGDKLCAIPNLRENYGELADCC |
| | | TKQEPERNECFLQHKDDNPSLPPFERPEAEAMCTSFKENPTTFMGHYLHEVARR |
| | | HPYFYAPELLYYAEQYNEILTQCCAEADKESCLTPKLDGVKEKALVSSVRQRMK |
| | | CSSMQKFGERAFKAWAVARLSQTFPNADFAEITKLATDLTKVNKECCHGDLLE |
| | | CADDRAELAKYMCENQATISSKLQTCCDKPLLKKAHCLSEVEHDTMPADLPAIA |
| | | ADFVEDQEVCKNYAEAKDVFLGTFLYEYSRRHPDYSVSLLLRLAKKYEATLEK |
| | | CCAEANPPACYGTVLAEFQPLVEEPKNLVKTNCDLYEKLGEYGFQNAILVRYTQ |
| | | KAPQVSTPTLVEAARNLGRVGTKCCTLPEDQRLPCVEDYLSAILNRVCLLHEKTP |
| | | VSEHVTKCCSGSLVERRPCFSALTVDETYVPKEFKAETFTFHSDICTLPEKEKQIK |
| | | KQTALAELVKHKPKATAEQLKTVMDDFAQFLDTCCKAADKDTCFSTEGPNLVT |
| | | RCKDALAHHHHHHEPEA |
| 109 | ACP75 | mdmrvpaqllgllllwlrgarcEAHKSEIAHRYNDLGEQHFKGLVLIAFSQYLQKCSYDEHA |
| | Mouse | KLVQEVTDFAKTCVADESAANCDKSLHTLFGDKLCAIPNLRENYGELADCCTK |
| | IFG | QEPERNECFLQHKDDNPSLPPFERPEAEAMCTSFKENPTTFMGHYLHEVARRHP |
| | fusion | YFYAPELLYYAEQYNEILTQCCAEADKESCLTPKLDGVKEKALVSSVRQRMKCS |
| | protein | SMQKFGERAFKAWAVARLSQTFPNADFAEITKLATDLTKVNKECCHGDLLECA |
| | | DDRAELAKYMCENQATISSKLQTCCDKPLLKKAHCLSEVEHDTMPADLPAIAAD |
| | | FVEDQEVCKNYAEAKDVFLGTFLYEYSRRHPDYSVSLLLRLAKKYEATLEKCCA |
| | | EANPPACYGTVLAEFQPLVEEPKNLVKTNCDLYEKLGEYGFQNAILVRYTQKAP |
| | | QVSTPTLVEAARNLGRVGTKCCTLPEDQRLPCVEDYLSAILNRVCLLHEKTPVSE |
| | | HVTKCCSGSLVERRPCFSALTVDETYVPKEFKAETFTFHSDICTLPEKEKQIKKQT |
| | | ALAELVKHKPKATAEQLKTVMDDFAQFLDTCCKAADKDTCFSTEGPNLVTRCK |
| | | DALASGGPGPAGMKGLPGShgtviesleslnnyfnssgidveekslfldiwrnwqkdgdmkilqsqiisfylr |
| | | lfevlkdnqaisnnisvieshlittffsnskakkdafmsiakfevnnpqvqrqafnelirvvhqllpesslrkrkrsrcSGGP |
| | | GPAGMKGLPGSggggsggggsEAHKSEIAHRYNDLGEQHFKGLVLIAFSQYLQKCSY |
| | | DEHAKLVQEVTDFAKTCVADESAANCDKSLHTLFGDKLCAIPNLRENYGELAD |
| | | CCTKQEPERNECFLQHKDDNPSLPPFERPEAEAMCTSFKENPTTFMGHYLHEVA |
| | | RRHPYFYAPELLYYAEQYNEILTQCCAEADKESCLTPKLDGVKEKALVSSVRQR |
| | | MKCSSMQKFGERAFKAWAVARLSQTFPNADFAEITKLATDLTKVNKECCHGDL |
| | | LECADDRAELAKYMCENQATISSKLQTCCDKPLLKKAHCLSEVEHDTMPADLP |
| | | AIAADFVEDQEVCKNYAEAKDVFLGTFLYEYSRRHPDYSVSLLLRLAKKYEATL |
| | | EKCCAEANPPACYGTVLAEFQPLVEEPKNLVKTNCDLYEKLGEYGFQNAILVRY |
| | | TQKAPQVSTPTLVEAARNLGRVGTKCCTLPEDQRLPCVEDYLSAILNRVCLLHE |
| | | KTPVSEHVTKCCSGSLVERRPCFSALTVDETYVPKEFKAETFTFHSDICTLPEKEK |
| | | QIKKQTALAELVKHKPKATAEQLKTVMDDFAQFLDTCCKAADKDTCFSTEGPN |
| | | LVTRCKDALAggggsggggsSGGPGPAGMKGLPGShgtviesleslnnyfnssgidveekslfldiwrn |
| | | wqkdgdmkilqsqiisfylrlfevlkdnqaisnnisvieshlittffsnskakkdafmsiakfevnnpqvqrqafnelirvvh |
| | | qllpesslrkrkrsrcSGGPGPAGMKGLPGSEAHKSEIAHRYNDLGEQHFKGLVLIAFSQY |
| | | LQKCSYDEHAKLVQEVTDFAKTCVADESAANCDKSLHTLFGDKLCAIPNLREN |
| | | YGELADCCTKQEPERNECFLQHKDDNPSLPPFERPEAEAMCTSFKENPTTFMGH |
| | | YLHEVARRHPYFYAPELLYYAEQYNEILTQCCAEADKESCLTPKLDGVKEKALV |
| | | SSVRQRMKCSSMQKFGERAFKAWAVARLSQTFPNADFAEITKLATDLTKVNKE |
| | | CCHGDLLECADDRAELAKYMCENQATISSKLQTCCDKPLLKKAHCLSEVEHDT |
| | | MPADLPAIAADFVEDQEVCKNYAEAKDVFLGTFLYEYSRRHPDYSVSLLLRLAK |
| | | KYEATLEKCCAEANPPACYGTVLAEFQPLVEEPKNLVKTNCDLYEKLGEYGFQ |
| | | NAILVRYTQKAPQVSTPTLVEAARNLGRVGTKCCTLPEDQRLPCVEDYLSAILNR |
| | | VCLLHEKTPVSEHVTKCCSGSLVERRPCFSALTVDETYVPKEFKAETFTFHSDICT |
| | | LPEKEKQIKKQTALAELVKHKPKATAEQLKTVMDDFAQFLDTCCKAADKDTCF |
| | | STEGPNLVTRCKDALAHHHHHHEPEA |
| 110 | ACP78 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFG | YYCTIGGSLSVSSQGTLVTVSSggggsggggsggggshgtviesleslnnyfnssgidveekslfldiwrn |
| | fusion | wqkdgdmkilqsqiisfylrlfevlkdnqaisnnisvieshlittffsnskakkdafmsiakfevnnpqvqrqafnelirvvh |
| | protein | qllpesslrkrkrsrcggggsggggsggggsEVQLVESGGGLVQPGNSLRLSCAASGFTFSKFGM |
| | | SWVRQAPGKGLEWVSSISGSGRDTLYAESVKGRFTISRDNAKTTLYLQMNSLRP |
| | | EDTAVYYCTIGGSLSVSSQGTLVTVSSggggsggggsggggshgtviesleslnnyfnssgidveeksl |
| | | fldiwrnwqkdgdmkilqsqiisfylrlfevlkdnqaisnnisvieshlittffsnskakkdafmsiakfevnnpqvqrqafn |
| | | elirvvhqllpesslrkrkrsrcggggsggggsggggsEVQLVESGGGLVQPGNSLRLSCAASGFTFS |
| | | KFGMSWVRQAPGKGLEWVSSISGSGRDTLYAESVKGRFTISRDNAKTTLYLQM |
| | | NSLRPEDTAVYYCTIGGSLSVSSQGTLVTVSSHHEIHRHEPEA |
| 111 | ACP134 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFG | YYCTIGGSLSVSSQGTLVTVSSSGGPGPAGMKGLPGShgtviesleslnnyfnssgidveekslfl |
| | fuision | diwrnwqkdgdmkilqsqiisfylrlfevlkdnqaisnnisvieshlittffsnskakkdafmsiakfevnnpqvqrqafnel |
| | protein | irvvhqllpesslrkrkrsrcSGGPGPAGMKGLPGSEVQLVESGGGLVQPGNSLRLSCAASG |
| | | FTFSKFGMSWVRQAPGKGLEWVSSISGSGRDTLYAESVKGRFTISRDNAKTTLY |
| | | LQMNSLRPEDTAVYYCTIGGSLSVSSQGTLVTVSSSGGPGPAGMKGLPGShgtvies |
| | | leslnnyfnssgidveekslfldiwrnwqkdgdmkilqsqiisfylrlfevlkdnqaisnnisvieshlittffsnskakkdaf |
| | | msiakfevnnpqvqrqafnelirvvhqllpesslrkrkrsrcSGGPGPAGMKGLPGSEVQLVESGGGL |
| | | VQPGNSLRLSCAASGFTFSKFGMSWVRQAPGKGLEWVSSISGSGRDTLYAESVK |
| | | GRFTISRDNAKTTLYLQMNSLRPEDTAVYYCTIGGSLSVSSQGTLVTVSSggggsgg |
| | | ggsggggsQVQLQESGGGLAQAGGSLSLSCAASGFTVSNSVMAWYRQTPGKQREF |
| | | VAIINSVGSTNYADSVKGRFTISRDNAKNTVYLQMNNLKPEDTAVYVCNRNFDR |
| | | IYWGQGTQVTVSSHHHHHHEPEA |
| 112 | ACP135 | mdmrvpaqllgllllwlrgarcQVQLQESGGGLAQAGGSLSLSCAASGFTVSNSVMAWYR |
| | Mouse | QTPGKQREFVAIINSVGSTNYADSVKGRFTISRDNAKNTVYLQMNNLKPEDTAV |
| | IFG | YVCNRNFDRIYWGQGTQVTVSSggggsggggsggggsEVQLVESGGGLVQPGNSLRLS |
| | fusion | CAASGFTFSKFGMSWVRQAPGKGLEWVSSISGSGRDTLYAESVKGRFTISRDNA |
| | protein | KTTLYLQMNSLRPEDTAVYYCTIGGSLSVSSQGTLVTVSSSGGPGPAGMKGLPG |
| | | Shgtviesleslnnyfnssgidveekslfldiwrnwqkdgdmkilqsqiisfylrlfevlkdnqaisnnisvieshlittffsnsk |
| | | akkdafmsiakfevnnpqvqrqafnelirvvhqllpesslrkrkrsrcSGGPGPAGMKGLPGSEVQLVES |
| | | GGGLVQPGNSLRLSCAASGFTFSKFGMSWVRQAPGKGLEWVSSISGSGRDTLYA |
| | | ESVKGRFTISRDNAKTTLYLQMNSLRPEDTAVYYCTIGGSLSVSSQGTLVTVSSS |
| | | GGPGPAGMKGLPGShgtviesleslnnyfnssgidveekslfldiwrnwqkdgdmkilqsqiisfylrlfevlkdn |
| | | qaisnnisvieshlittffsnskakkdafmsiakfevnnpqvqrqafnelirvvhqllpesslrkrkrsrcSGGPGPAGM |
| | | KGLPGSEVQLVESGGGLVQPGNSLRLSCAASGFTFSKFGMSWVRQAPGKGLEW |
| | | VSSISGSGRDTLYAESVKGRFTISRDNAKTTLYLQMNSLRPEDTAVYYCTIGGSL |
| | | SVSSQGTLVTVSSHHHHHHEPEA |
| 113 | ACP34 | mdmrvpaqllgllllwlrgarcrvipvsgparclsqsrnllkttddmvktareklkhysctaedidheditrdqtstlktclplel |
| | Mouse | hknesclatretssttrgsclppqktslmmtlclgsiyedlkmyqtefqainaalqnhnhqqiildkgmlvaidelmqslnhn |
| | IL-12 | getlrqkppvgeadpyrvkmklcillhafstrvvtinrvmgylssaSGGPGPAGMKGLPGSmwelekdvyvv |
| | fusion | evdwtpdapgetvnltcdtpeedditwtsdqrhgvigsgktltitvkefldagqytchkggetlshshlllhkkengiwsteil |
| | protein | knfknktflkceapnysgrftcswlvqrnmdlkfnikssssspdsravtcgmaslsaekvtldqrdyekysvscqedvtcpt |
| | | aeetlpielalearqqnkyenystsffirdiikpdppknlqmkplknsqvevsweypdswstphsyfslkffvriqrkkek |
| | | mketeegcnqkgaflvektstevqckggnvcvqaqdryynsscskwacvpcrvrsHHHHHH |
| 114 | ACP35 | mdmrvpaqllgllllwlrgarcrvipvsgparclsqsrnllkttddmvktareklkhysctaedidheditrdqtstlktclplel |
| | Mouse | hknesclatretssttrgsclppqktslmmtlclgsiyedlkmyqtefqainaalqnhnhqqiildkgmlvaidelmqslnhn |
| | IL-12 | getlrqkppvgeadpyrvkmklcillhafstrvvtinrvmgylssaggggsggggsggggsSGGPGPAGMKGLP |
| | fusion | GSggggsggggsggggsmwelekdvyvvevdwtpdapgetvnltcdtpeedditwtsdqrhgvigsgktltitvkefld |
| | protein | agqytchkggetlshshlllhkkengiwsteilknfknktflkceapnysgrftcswlvqrnmdlkfnikssssspdsravtc |
| | | gmaslsaekvtldqrdyekysvscqedvtcptaeetlpielalearqqnkyenystsffirdiikpdppknlqmkplknsqv |
| | | evsweypdswstphsyfslkffvriqrkkekmketeegcnqkgaflvektstevqckggnvcvqaqdryynsscskwac |
| | | vpcrvrsHHHHHH |
| 115 | ACP36 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IL-12 | YYCTIGGSLSVSSQGTLVTVSSSGGPGPAGMKGLPGSmwelekdvyvvevdwtpdapgetv |
| | fusion | nltcdtpeedditwtsdqrhgvigsgktltitvkefldagqytchkggetlshshlllhkkengiwsteilknfknktflkceap |
| | protein | nysgrftcswlvqrnmdlkfnikssssspdsravtcgmaslsaekvtldqrdyekysvscqedvtcptaeetlpielalearq |
| | | qnkyenystsffirdiikpdppknlqmkplknsqvevsweypdswstphsyfslkffvriqrkkekmketeegcnqkga |
| | | flvektstevqckggnvcvqaqdryynsscskwacypcrvrsggggsggggsggggsrvipvsgparclsqsrnllkttdd |
| | | mvktareklkhysctaedidheditrdqtstlktclplelhknesclatretssttrgsclppqktslmmtlclgsiyedlkmyqt |
| | | efqainaalqnhnhqqiildkgmlvaidelmqslnhngetlrqkppvgeadpyrvkmklcillhafstrvvtinrvmgylss |
| | | aSGGPGPAGMKGLPGSEVQLVESGGGLVQPGNSLRLSCAASGFTFSKFGMSWVR |
| | | QAPGKGLEWVSSISGSGRDTLYAESVKGRFTISRDNAKTTLYLQMNSLRPEDTA |
| | | VYYCTIGGSLSVSSQGTLVTVSSHHHHHH |
| 116 | ACP37 | mdmrvpaqllgllllwlrgarcQVQLQESGGGLVQAGGSLRLSCAASGRIFSIDIMSWYRQA |
| | Mouse | PGKQRELVARITRGGTISYDDSVKGRFTISRDNAKNTVYLQMNSLKPEDTGVYY |
| | IL-12 | CNALYGTDYWGKGTQVTVSSggggsggggsggggsEVQLVESGGGLVQPGNSLRLSC |
| | fusion | AASGFTFSKFGMSWVRQAPGKGLEWVSSISGSGRDTLYAESVKGRFTISRDNAK |
| | protein | TTLYLQMNSLRPEDTAVYYCTIGGSLSVSSQGTLVTVSSSGGPGPAGMKGLPGS |
| | | mwelekdvyvvevdwtpdapgetvnltcdtpeedditwtsdqrhgvigsgktltitvkefldagqytchkggetlshshlll |
| | | hkkengiwsteilknfknktflkceapnysgrftcswlyqrnmdlkfnikssssspdsravtcgmaslsaekvtldqrdyek |
| | | ysvscqedvtcptaeetlpielalearqqnkyenystsffirdiikpdppknlqmkplknsqveysweypdswstphsyfsl |
| | | kffvriqrkkekmketeegcnqkgaflvektstevqckggnvcvqaqdryynsscskwacvpcrvrsggggsggggsg |
| | | gggsrvipvsgparclsqsrnllkttddmvktareklkhysctaedidheditrdqtstlktclplelhknesclatretssttrgsc |
| | | lppqktslmmtlclgsiyedlkmyqtefqainaalqnhnhqqiildkgmlvaidelmqslnhngetlrqkppvgeadpyr |
| | | vkmklcillhafstrvvtinrvmgylssaSGGPGPAGMKGLPGSEVQLVESGGGLVQPGNSLRL |
| | | SCAASGFTFSKFGMSWVRQAPGKGLEWVSSISGSGRDTLYAESVKGRFTISRDN |
| | | AKTTLYLQMNSLRPEDTAVYYCTIGGSLSVSSQGTLVTVSSHHHHHH |
| 117 | ACP79 | mdmrvpaqllgllllwlrgarcQVQLQESGGGLVQAGGSLRLSCAASGRIFSIDIMSWYRQA |
| | Mouse | PGKQRELVARITRGGTISYDDSVKGRFTISRDNAKNTVYLQMNSLKPEDTGVYY |
| | IL-12 | CNALYGTDYWGKGTQVTVSSggggsggggsggggsEVQLVESGGGLVQPGNSLRLSC |
| | fusion | AASGFTFSKFGMSWVRQAPGKGLEWVSSISGSGRDTLYAESVKGRFTISRDNAK |
| | protein | TTLYLQMNSLRPEDTAVYYCTIGGSLSVSSQGTLVTVSSSGGPGPAGMKGLPGS |
| | | mwelekdvyvvevdwtpdapgetvnltcdtpeedditwtsdqrhgvigsgktltitykefldagqytchkggetlshshlll |
| | | hkkengiwsteilknfknktflkceapnysgrftcswlvqrnmdlkfnikssssspdsravtcgmaslsaekvtldqrdyek |
| | | ysvscqedvtcptaeetlpielalearqqnkyenystsffirdiikpdppknlqmkplknsqvevsweypdswstphsyfsl |
| | | kffvriqrkkekmketeegcnqkgaflvektstevqckggnvcvqaqdryynsscskwacvpcrvrsggggsggggsg |
| | | gggsrvipvsgparclsqsrnllkttddmvktareklkhysctaedidheditrdqtstlktclplelhknesclatretssttrgsc |
| | | lppqktslmmtlclgsiyedlkmyqtefqainaalqnhnhqqiildkgmlvaidelmqslnhngetlrqkppvgeadpyr |
| | | vkmklcillhafstrvvtinrvmgylssaSGGPGPAGMKGLPGSEVQLVESGGGLVQPGNSLRL |
| | | SCAASGFTFSKFGMSWVRQAPGKGLEWVSSISGSGRDTLYAESVKGRFTISRDN |
| | | AKTTLYLQMNSLRPEDTAVYYCTIGGSLSVSSQGTLVTVSSHHHHHH |
| 118 | ACP80 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IL-12 | YYCTIGGSLSVSSQGTLVTVSSSGGPGPAGMKGLPGSmwelekdvyvvevdwtpdapgetv |
| | fusion | nltcdtpeedditwtsdqrhgvigsgktltitvkefldagqytchkggetlshshlllhkkengiwsteilknfknktflkceap |
| | protein | nysgrftcswlvqrnmdlkfnikssssspdsravtcgmaslsaekvtldqrdyekysvscqedvtcptaeetlpielalearq |
| | | qnkyenystsffirdiikpdppknlqmkplknsqvevsweypdswstphsyfslkffvriqrkkekmketeegcnqkga |
| | | flvektstevqckggnvcvqaqdryynsscskwacvpcrvrsggggsggggsggggsrvipvsgparclsqsrnllkttdd |
| | | myktareklkhysctaedidheditrdqtstlktclplelhknesclatretssttrgsclppqktslmmtlclgsiyedlkmyqt |
| | | efqainaalqnhnhqqiildkgmlvaidelmqslnhngetlrqkppvgeadpyrvkmklcillhafstrvvtinrvmgylss |
| | | aSGGPGPAGMKGLPGSEVQLVESGGGLVQPGNSLRLSCAASGFTFSKFGMSWVR |
| | | QAPGKGLEWVSSISGSGRDTLYAESVKGRFTISRDNAKTTLYLQMNSLRPEDTA |
| | | VYYCTIGGSLSVSSQGTLVTVSSggggsggggsggggsQVQLQESGGGLVQAGGSLRLS |
| | | CAASGRIFSIDIMSWYRQAPGKQRELVARITRGGTISYDDSVKGRFTISRDNAKN |
| | | TVYLQMNSLKPEDTGVYYCNALYGTDYWGKGTQVTVSSHHHHHH |
| 119 | ACP91 | mdmrvpaqllgllllwlrgarciwelkkdvyvveldwypdapgemvvltcdtpeedgitwtldqssevlgsgktltiqvke |
| | Chimeric | fgdagqytchkggevlshsllllhkkedgiwstdilkdqkepknktflrceaknysgrftcwwlttistdltfsvkssrgssdp |
| | IL-12 | qgvtcgaatlsaervrgdnkeyeysvecqedsacpaaeeslpievmvdavhklkyenytssffirdiikpdppknlqlkplk |
| | fusion | nsrqvevsweypdtwstphsyfsltfcvqvqgkskrekkdrvftdktsatvicrknasisvraqdryyssswsewasvpcs |
| | protein | ggggsggggsggggsrvipvsgparclsqsrnllkttddmvktareklkhysctaedidheditrdqtstlktclplelhknes |
| | | clatretssttrgsclppqktslmmtlclgsiyedlkmyqtefqainaalqnhnhqqiildkgmlvaidelmqslnhngetltrq |
| | | kppvgeadpyrvkmklcillhafstrvvtinrvmgylssaggggsggggsggggsggggsggggsggggsggggsggg |
| | | gsggggsQSVLTQPPSVSGAPGQRVTISCSGSRSNIGSNTVKWYQQLPGTAPKLLIY |
| | | YNDQRPSGVPDRFSGSKSGTSASLAITGLQAEDEADYYCQSYDRYTHPALLFGT |
| | | GTKVTVLggggsggggsggggsQVQLVESGGGVVQPGRSLRLSCAASGFTFSSYGMH |
| | | WVRQAPGKGLEWVAFIRYDGSNKYYADSVKGRFTISRDNSKNTLYLQMNSLRA |
| | | EDTAVYYCKTHGSHDNWGQGTMVTVSSggggsggggsggggsEVQLVESGGGLVQP |
| | | GNSLRLSCAASGFTFSKFGMSWVRQAPGKGLEWVSSISGSGRDTLYAESVKGRF |
| | | TISRDNAKTTLYLQMNSLRPEDTAVYYCTIGGSLSVSSQGTLVTVSSHHHHHHEP |
| | | EA |
| 120 | ACP136 | mdmrvpaqllgllllwlrgarciwelkkdvyvveldwypdapgemvvltcdtpeedgitwtldqssevlgsgktltiqvke |
| | Chimeric | fgdagqytchkggevlshsllllhkkedgiwstdilkdqkepknktflrceaknysgrftcwwlttistdltfsvkssrgssdp |
| | IL-12 | qgvtcgaatlsaervrgdnkeyeysvecqedsacpaaeeslpievmvdavhklkyenytssffirdiikpdppknlqlkplk |
| | fusion | nsrqvevsweypdtwstphsyfsltfcvqvqgkskrekkdrvftdktsatvicrknasisvraqdryyssswsewasvpcs |
| | protein | ggggsggggsggggsrvipvsgparclsqsrnllkttddmvktareklkhysctaedidheditrdqtstlktclplelhknes |
| | | clatretssttrgsclppqktslmmticlgsiyedlkmyqtefqainaalqnhnhqqiildkgmlvaidelmqslnhngetlrq |
| | | kppvgeadpyrvkmklcillhafstrvvtinrvmgylssaSGGPGPAGMKGLPGSggggsggggsggggsgg |
| | | ggsggggsggggsQSVLTQPPSVSGAPGQRVTISCSGSRSNIGSNTVKWYQQLPGTAP |
| | | KLLIYYNDQRPSGVPDRFSGSKSGTSASLAITGLQAEDEADYYCQSYDRYTHPAL |
| | | LFGTGTKVTVLggggsggggsggggsQVQLVESGGGVVQPGRSLRLSCAASGFTFSSY |
| | | GMHWVRQAPGKGLEWVAFIRYDGSNKYYADSVKGRFTISRDNSKNTLYLQMN |
| | | SLRAEDTAVYYCKTHGSHDNWGQGTMVTVSSHREIREIHEPEA |
| 121 | ACP138 | mdmrvpaqllgllllwlrgarciwelkkdvyvveldwypdapgemvvltcdtpeedgitwtldqssevlgsgktltiqvke |
| | Chimeric | fgdagqytchkggevlshsllllhkkedgiwstdilkdqkepknktflrceaknysgrftcwwlttistdltfsvkssrgssdp |
| | IL-12 | qgvtcgaatlsaervrgdnkeyeysvecqedsacpaaeeslpievmvdavhklkyenytssffirdiikpdppknlqlkplk |
| | fusion | nsrqvevsweypdtwstphsyfsltfcvqvqgkskrekkdrvftdktsatvicrknasisvraqdryyssswsewasvpcs |
| | protein | ggggsggggsggggsrvipvsgparclsqsrnllkttddmvktareklkhysctaedidheditrdqtstlktclplelhknes |
| | | clatretssttrgsclppqktslmmtlclgsiyedlkmyqtefqainaalqnhnhqqiildkgmlvaidelmqslnhngetlrq |
| | | kppvgeadpyrvkmklcillhafstrvvtinrvmgylssaSGGPGPAGMKGLPGSggggsggggsggggsgg |
| | | ggsggggsggggsQSVLTQPPSVSGAPGQRVTISCSGSRSNIGSNTVKWYQQLPGTAP |
| | | KLLIYYNDQRPSGVPDRFSGSKSGTSASLAITGLQAEDEADYYCQSYDRYTHPAL |
| | | LFGTGTKVTVLggggsggggsggggsQVQLVESGGGVVQPGRSLRLSCAASGFTFSSY |
| | | GMHWVRQAPGKGLEWVAFIRYDGSNKYYADSVKGRFTISRDNSKNTLYLQMN |
| | | SLRAEDTAVYYCKTHGSHDNWGQGTMVTVSSggggsggggsggggsEVQLVESGGGL |
| | | VQPGNSLRLSCAASGFTFSKFGMSWVRQAPGKGLEWVSSISGSGRDTLYAESVK |
| | | GRFTISRDNAKTTLYLQMNSLRPEDTAVYYCTIGGSLSVSSQGTLVTVSSggggsgg |
| | | ggsggggsQVQLQESGGGLAQAGGSLSLSCAASGFTVSNSVMAWYRQTPGKQREF |
| | | VAIINSVGSTNYADSVKGRFTISRDNAKNTVYLQMNNLKPEDTAVYVCNRNFDR |
| | | IYWGQGTQVTVSSHEIREIHHEPEA |
| 122 | ACP139 | mdmrvpaqllgllllwlrgarcQVQLQESGGGLAQAGGSLSLSCAASGFTVSNSVMAWYR |
| | Chimeric | QTPGKQREFVAIINSVGSTNYADSVKGRFTISRDNAKNTVYLQMNNLKPEDTAV |
| | IL-12 | YVCNRNFDRIYWGQGTQVTVSSggggsggggsggggsiwelkkdvyvveldwypdapgemvvltcd |
| | fusion | tpeedgitwtldqssevlgsgktltiqvkefgdagqytchkggevlshsllllhkkedgiwstdilkdqkepknktflrceakn |
| | protein | ysgrftcwwlttistdltfsvkssrgssdpqgvtcgaatlsaervrgdnkeyeysvecqedsacpaaeeslpievmvdavhkl |
| | | kyenytssffirdiikpdppknlqlkplknsrqvevsweypdtwstphsyfsltfcvqvqgkskrekkdrvftdktsatvicr |
| | | knasisvraqdryyssswsewasvpcsggggsggggsggggsrvipvsgparclsqsrnllkttddmvktareklkhysct |
| | | aedidheditrdqtstlktclplelhknesclatretssttrgsclppqktslmmtlclgsiyedlkmyqtefqainaalqnhnhq |
| | | qiildkgmlvaidelmqslnhngetlrqkppvgeadpyrvkmklcillhafstrvvtinrvmgylssaSGGPGPAGM |
| | | KGLPGSggggsggggsggggsggggsggggsggggsQSVLTQPPSVSGAPGQRVTISCSGSRS |
| | | NIGSNTVKWYQQLPGTAPKLLIYYNDQRPSGVPDRFSGSKSGTSASLAITGLQAE |
| | | DEADYYCQSYDRYTHPALLFGTGTKVTVLggggsggggsggggsQVQLVESGGGVVQ |
| | | PGRSLRLSCAASGFTFSSYGMHWVRQAPGKGLEWVAFIRYDGSNKYYADSVKG |
| | | RFTISRDNSKNTLYLQMNSLRAEDTAVYYCKTHGSHDNWGQGTMVTVSSggggs |
| | | ggggsggggsEVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVYYCTIGGS |
| | | LSVSSQGTLVTVSSHHHHHHEPEA |
| 123 | ACP140 | mdmrvpaqllgllllwlrgarcQVQLQESGGGLAQAGGSLSLSCAASGFTVSNSVMAWYR |
| | Chimeric | QTPGKQREFVAIINSVGSTNYADSVKGRFTISRDNAKNTVYLQMNNLKPEDTAV |
| | IL-12 | YVCNRNFDRIYWGQGTQVTVSSSGGPGPAGMKGLPGSiwelkkdvyvveldwypdapge |
| | fusion | mvvltcdtpeedgitwtldqssevlgsgktltiqvkefgdagqytchkggevlshsllllhkkedgiwstdilkdqkepknkt |
| | protein | flrceaknysgrftcwwlttistdltfsvkssrgssdpqgvtcgaatlsaervrgdnkeyeysvecqedsacpaaeeslpiev |
| | | mvdavhklkyenytssffirdiikpdppknlqlkplknsrqvevsweypdtwstphsyfsltfcvqvqgkskrekkdrvft |
| | | dktsatvicrknasisvraqdryyssswsewasvpcsggggsggggsggggsrvipvsgparclsqsrnllkttddmvkta |
| | | reklkhysctaedidheditrdqtstlktclplelhknesclatretssttrgsclppqktslmmtlclgsiyedlkmyqtefqain |
| | | aalqnhnhqqiildkgmlvaidelmqslnhngetlrqkppvgeadpyrvkmklcillhafstrvvtinrvmgylssaSGG |
| | | PGPAGMKGLPGSggggsggggsggggsggggsggggsggggsQSVLTQPPSVSGAPGQRVTIS |
| | | CSGSRSNIGSNTVKWYQQLPGTAPKLLIYYNDQRPSGVPDRFSGSKSGTSASLAI |
| | | TGLQAEDEADYYCQSYDRYTHPALLFGTGTKVTVLggggsggggsggggsQVQLVES |
| | | GGGVVQPGRSLRLSCAASGFTFSSYGMHWVRQAPGKGLEWVAFIRYDGSNKY |
| | | YADSVKGRFTISRDNSKNTLYLQMNSLRAEDTAVYYCKTHGSHDNWGQGTMV |
| | | TVSSggggsggggsggggsEVQLVESGGGLVQPGNSLRLSCAASGFTFSKFGMSWVRQ |
| | | APGKGLEWVSSISGSGRDTLYAESVKGRFTISRDNAKTTLYLQMNSLRPEDTAV |
| | | YYCTIGGSLSVSSQGTLVTVSSHHHHHHEPEA |
| 124 | ACP38 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSEVQLVESGGGLVQPGGSLRLSCAASGFTFSSYTLAWVRQAPGKGLEWV |
| | protein | AAIDSSSYTYSPDTVRGRFTISRDNAKNSLYLQMNSLRAEDTAVYYCARDSNWD |
| | | ALDYWGQGTTVTVSSGGGGSGGGGSGGGGSDIQMTQSPSSLSASVGDRVTITCK |
| | | ASQNVGTNVGWYQQKPGKAPKALIYSASFRYSGVPSRFSGSGSGTDFTLTISSLQ |
| | | PEDFATYYCQQYYTYPYTFGGGTKVEIKggggsggggsggggsEVQLVESGGGLVQPG |
| | | NSLRLSCAASGFTFSKFGMSWVRQAPGKGLEWVSSISGSGRDTLYAESVKGRFTI |
| | | SRDNAKTTLYLQMNSLRPEDTAVYYCTIGGSLSVSSQGTLVTVSSggggsggggsggg |
| | | gsQVQLQESGGGLVQAGGSLRLSCAASGRIFSIDIMSWYRQAPGKQRELVARITR |
| | | GGTISYDDSVKGRFTISRDNAKNTVYLQMNSLKPEDTGVYYCNALYGTDYWGK |
| | | GTQVTVSSHHHHHH |
| 125 | ACP39 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSSGGPGPAGMKGLPGSEVQLVESGGGLVQPGNSL |
| | protein | RLSCAASGFTFSKFGMSWVRQAPGKGLEWVSSISGSGRDTLYAESVKGRFTISR |
| | | DNAKTTLYLQMNSLRPEDTAVYYCTIGGSLSVSSQGTLVTVSSSGGPGPAGMKG |
| | | LPGSEVQLVESGGGLVQPGGSLRLSCAASGFTFSSYTLAWVRQAPGKGLEWVA |
| | | AIDSSSYTYSPDTVRGRFTISRDNAKNSLYLQMNSLRAEDTAVYYCARDSNWDA |
| | | LDYWGQGTTVTVSSGGGGSGGGGSGGGGSDIQMTQSPSSLSASVGDRVTITCKA |
| | | SQNVGTNVGWYQQKPGKAPKALIYSASFRYSGVPSRFSGSGSGTDFTLTISSLQP |
| | | EDFATYYCQQYYTYPYTFGGGTKVEIKSGGPGPAGMKGLPGSaptssstkktqlqlehllld |
| | | lqmilnginnyknpkltrmltffympkkatelkhlqcleeelkpleevlnlaqsknfhlrprdlisninvivlelkgsettfm |
| | | ceyadetativeflnrwitfcqsiistltHHHHHH** |
| 126 | ACP40 | mdmrvpaqllgllllwlrgarcelcdddppeiphatfkamaykegtmlnceckrgfrriksgslymlctgnsshsswdnq |
| | IL-2 | cqctssatrnttkqvtpqpeeqkerkttemqspmqpvdqaslpghcrepppweneateriyhfvvgqmvyyqcvqgyr |
| | fusion | alhrgpaesvckmthgktrwtqpqlictgemetsqfpgeekpqaspegrpesetsclvtttdfqiqtemaatmetsiftteyq |
| | protein | ggggsggggsggggsggggsggggsggggsSGGPGPAGMKGLPGSaptssstkktqlqlehllldlqmilngi |
| | | nnyknpkltrmltfkfympkkatelkhlqcleeelkpleevlnlaqsknfhlrprdlisninvivlelkgsettfmceyadetat |
| | | iveflnrwitfcqsiistltHHHHHH |
| 127 | ACP41 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSggggsggggsggggsggggsggggsggggselcdddppeiphatfkamaykegtmlnceckrgfrriksgs |
| | protein | lymlctgnsshsswdnqcqctssatrnttkqvtpqpeeqkerkttemqspmqpvdqaslpghcrepppweneateriyhf |
| | | vvgqmvyyqcvqgyralhrgpaesvckmthgktrwtqpqlictgemetsqfpgeekpqaspegrpesetsclvtttdfqi |
| | | qtemaatmetsiftteyqHHHHHH |
| 128 | ACP42 | mdmrvpaqllgllllwlrgarcEVQLVESGGGLVQPGNSLRLSCAASGFTFSKFGMSWVRQ |
| | IL-2 | APGKGLEWVSSISGSGRDTLYAESVKGRFTISRDNAKTTLYLQMNSLRPEDTAV |
| | fusion | YYCTIGGSLSVSSQGTLVTVSSggggsggggsggggselcdddppeiphatfkamaykegtmlnceckr |
| | protein | gfrriksgslymlctgnsshsswdnqcqctssatrnttkqvtpqpeeqkerkttemqspmqpvdqaslpghcrepppwen |
| | | eateriyhfvvgqmvyyqcvqgyralhrgpaesvckmthgktrwtqpqlictgemetsqfpgeekpqaspegrpesets |
| | | clvtttdfqiqtemaatmetsiftteyqggggsggggsggggsggggsggggsggggsSGGPGPAGMKGLPGS |
| | | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltHHHHHH |
| 129 | ACP43 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSggggsggggsggggsggggsggggsggggselcdddppeiphatfkamaykegtmlnceckrgfrriksgs |
| | protein | lymlctgnsshsswdnqcqctssatrnttkqvtpqpeeqkerkttemqspmqpvdqaslpghcrepppweneateriyhf |
| | | vvgqmvyyqcvqgyralhrgpaesvckmthgktrwtqpqlictgemetsqfpgeekpqaspegrpesetsclvtttdfqi |
| | | qtemaatmetsiftteyqggggsggggsggggsEVQLVESGGGLVQPGNSLRLSCAASGFTFSKF |
| | | GMSWVRQAPGKGLEWVSSISGSGRDTLYAESVKGRFTISRDNAKTTLYLQMNS |
| | | LRPEDTAVYYCTIGGSLSVSSQGTLVTVSSHHHHHH |
| 130 | ACP44 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSggggsggggsggggsggggsggggsggggselcdddppeiphatfkamaykegtmlnceckrgfrriksgs |
| | protein | lymlctgnsshsswdnqcqctssatrnttkqvtpqpeeqkerkttemqspmqpvdqaslpghcrepppweneateriyhf |
| | | vvgqmvyyqcvqgyralhrgpaesvckmthgktrwtqpqlictgemetsqfpgeekpqaspegrpesetsclvtttdfqi |
| | | qtemaatmetsiftteyqSGGPGPAGMKGLPGSEVQLVESGGGLVQPGNSLRLSCAASGF |
| | | TFSKFGMSWVRQAPGKGLEWVSSISGSGRDTLYAESVKGRFTISRDNAKTTLYL |
| | | QMNSLRPEDTAVYYCTIGGSLSVSSQGTLVTVSSFIRREIREI |
| 131 | ACP45 | mdmrvpaqllgllllwlrgarcEVQLVESGGGLVQPGNSLRLSCAASGFTFSKFGMSWVRQ |
| | IL-2 | APGKGLEWVSSISGSGRDTLYAESVKGRFTISRDNAKTTLYLQMNSLRPEDTAV |
| | fusion | YYCTIGGSLSVSSQGTLVTVSSSGGPGPAGMKGLPGSEVQLVESGGGLVQPGGS |
| | protein | LRLSCAASGFTFSSYTLAWVRQAPGKGLEWVAAIDSSSYTYSPDTVRGRFTISRD |
| | | NAKNSLYLQMNSLRAEDTAVYYCARDSNWDALDYWGQGTTVTVSSGGGGSG |
| | | GGGSGGGGSDIQMTQSPSSLSASVGDRVTITCKASQNVGTNVGWYQQKPGKAP |
| | | KALIYSASFRYSGVPSRFSGSGSGTDFTLTISSLQPEDFATYYCQQYYTYPYTFGG |
| | | GTKVEIKggggsggggsggggsggggsggggsggggsSGGPGPAGMKGLPGSaptssstkktqlqlehl |
| | | lldlqmilnginnyknpkltrmltfkfympkkatelkhlqcleeelkpleevlnlaqsknfhlrprdlisninvivlelkgsettf |
| | | mceyadetativeflnrwitfcqsiistltHHHHHH |
| 132 | ACP46 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSggggsggggsggggsggggsggggsggggsEVQLVESGGGLVQPGGSLRLSCAASGF |
| | protein | TFSSYTLAWVRQAPGKGLEWVAAIDSSSYTYSPDTVRGRFTISRDNAKNSLYLQ |
| | | MNSLRAEDTAVYYCARDSNWDALDYWGQGTTVTVSSsggpgpagmkglpgsDIQMT |
| | | QSPSSLSASVGDRVTITCKASQNVGTNVGWYQQKPGKAPKALIYSASFRYSGVP |
| | | SRFSGSGSGTDFTLTISSLQPEDFATYYCQQYYTYPYTFGGGTKVEIKggggsggggs |
| | | ggggsEVQLVESGGGLVQPGNSLRLSCAASGFTFSKFGMSWVRQAPGKGLEWVSS |
| | | ISGSGRDTLYAESVKGRFTISRDNAKTTLYLQMNSLRPEDTAVYYCTIGGSLSVS |
| | | SQGTLVTVSSggggsggggsggggsQVQLQESGGGLVQAGGSLRLSCAASGRIFSIDIM |
| | | SWYRQAPGKQRELVARITRGGTISYDDSVKGRFTISRDNAKNTVYLQMNSLKPE |
| | | DTGVYYCNALYGTDYWGKGTQVTVSSHHHHHH |
| 133 | ACP47 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSggggsggggsggggsaptssstkktqlqlehllldlqmilnginnyknpk |
| | protein | ltrmltfkfympkkatelkhlqcleeelkpleevlnlaqsknfhlrprdlisninvivlelkgsettfmceyadetativeflnrw |
| | | itfcqsiistltSGGPGPAGMKGLPGSEVQLVESGGGLVQPGNSLRLSCAASGFTFSKFG |
| | | MSWVRQAPGKGLEWVSSISGSGRDTLYAESVKGRFTISRDNAKTTLYLQMNSLR |
| | | PEDTAVYYCTIGGSLSVSSQGTLVTVSSggggsggggsggggsggggsggggsggggsEVQLV |
| | | ESGGGLVQPGGSLRLSCAASGFTFSSYTLAWVRQAPGKGLEWVAAIDSSSYTYS |
| | | PDTVRGRFTISRDNAKNSLYLQMNSLRAEDTAVYYCARDSNWDALDYWGQGT |
| | | TVTVSSGGGGSGGGGSGGGGSDIQMTQSPSSLSASVGDRVTITCKASQNVGTNV |
| | | GWYQQKPGKAPKALIYSASFRYSGVPSRFSGSGSGTDFTLTISSLQPEDFATYYC |
| | | QQYYTYPYTFGGGTKVEIKHHHHHH |
| 134 | ACP48 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSEVQLVESGGGLVQPGGSLRLSCAASGFTFSSYTLAWVRQAPGKGLEWV |
| | protein | AAIDSSSYTYSPDTVRGRFTISRDNAKNSLYLQMNSLRAEDTAVYYCARDSNWD |
| | | ALDYWGQGTTVTVSSGGGGSGGGGSGGGGSDIQMTQSPSSLSASVGDRVTITCK |
| | | ASQNVGTNVGWYQQKPGKAPKALIYSASFRYSGVPSRFSGSGSGTDFTLTISSLQ |
| | | PEDFATYYCQQYYTYPYTFGGGTKVEIKggggsggggsggggsEVQLVESGGGLVQPG |
| | | NSLRLSCAASGFTFSKFGMSWVRQAPGKGLEWVSSISGSGRDTLYAESVKGRFTI |
| | | SRDNAKTTLYLQMNSLRPEDTAVYYCTIGGSLSVSSQGTLVTVSSHHHHHH |
| 135 | ACP49 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSggggsggggsggggsggggsggggsggggsEVQLVESGGGLVQPGGSLRLSCAASGF |
| | protein | TFSSYTLAWVRQAPGKGLEWVAAIDSSSYTYSPDTVRGRFTISRDNAKNSLYLQ |
| | | MNSLRAEDTAVYYCARDSNWDALDYWGQGTTVTVSSGGGGSGGGGSGGGGS |
| | | DIQMTQSPSSLSASVGDRVTITCKASQNVGTNVGWYQQKPGKAPKALIYSASFR |
| | | YSGVPSRFSGSGSGTDFTLTISSLQPEDFATYYCQQYYTYPYTFGGGTKVEIKggg |
| | | gsggggsggggsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSHHHHHH |
| 136 | ACP92 | mdmrvpaqllgllllwlrgarcEVQLVESGGGLVQPGNSLRLSCAASGFTFSKFGMSWVRQ |
| | IL-2 | APGKGLEWVSSISGSGRDTLYAESVKGRFTISRDNAKTTLYLQMNSLRPEDTAV |
| | fusion | YYCTIGGSLSVSSQGTLVTVSSSGGPGPAGMKGLPGSaptssstkktqlqlehllldlqmilngin |
| | protein | nyknpkltrmltfkfympkkatelkhlqcleeelkpleevlnlaqsknfhlrprdlisninvivlelkgsettfmceyadetati |
| | | veflnrwitfcqsiistltSGGPGPAGMKGLPGSEVQLVESGGGLVQPGNSLRLSCAASGFT |
| | | FSKFGMSWVRQAPGKGLEWVSSISGSGRDTLYAESVKGRFTISRDNAKTTLYLQ |
| | | MNSLRPEDTAVYYCTIGGSLSVSSQGTLVTVSSHHHHHH |
| 137 | ACP93 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSgsgsgsgsgsgsgsgsEVQLVESGGGLVQPGNSLRLSC |
| | protein | AASGFTFSKFGMSWVRQAPGKGLEWVSSISGSGRDTLYAESVKGRFTISRDNAK |
| | | TTLYLQMNSLRPEDTAVYYCTIGGSLSVSSQGTLVTVSSgsgsgsgsgsgsgsgsQVQLQ |
| | | ESGGGLVQAGGSLRLSCAASGRIFSIDIMSWYRQAPGKQRELVARITRGGTISYD |
| | | DSVKGRFTISRDNAKNTVYLQMNSLKPEDTGVYYCNALYGTDYWGKGTQVTV |
| | | SSgsgsgsgsgsgsgsgsEVQLVESGGGLVQPGGSLRLSCAASGFTFSSYTLAWVRQAP |
| | | GKGLEWVAAIDSSSYTYSPDTVRGRFTISRDNAKNSLYLQMNSLRAEDTAVYYC |
| | | ARDSNWDALDYWGQGTTVTVSSGGGGSGGGGSGGGGSDIQMTQSPSSLSASVG |
| | | DRVTITCKASQNVGTNVGWYQQKPGKAPKALIYSASFRYSGVPSRFSGSGSGTD |
| | | FTLTISSLQPEDFATYYCQQYYTYPYTFGGGTKVEIKSGGPGPAGMKGLPGSaptss |
| | | stkktqlqlehllldlqmilnginnyknpkltrmltfkfympkkatelkhlqcleeelkpleevlnlaqsknfhlrprdlisnin |
| | | vivlelkgsettfmceyadetativeflnrwitfcqsiistltHHHHHH |
| 138 | ACP94 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSgsgsgsgsgsgsgsgsEVQLVESGGGLVQPGNSLRLSC |
| | protein | AASGFTFSKFGMSWVRQAPGKGLEWVSSISGSGRDTLYAESVKGRFTISRDNAK |
| | | TTLYLQMNSLRPEDTAVYYCTIGGSLSVSSQGTLVTVSSgsgsgsgsgsgsgsgsEVQLV |
| | | ESGGGLVQPGGSLRLSCAASGFTFSSYTLAWVRQAPGKGLEWVAAIDSSSYTYS |
| | | PDTVRGRFTISRDNAKNSLYLQMNSLRAEDTAVYYCARDSNWDALDYWGQGT |
| | | TVTVSSGGGGSGGGGSGGGGSDIQMTQSPSSLSASVGDRVTITCKASQNVGTNV |
| | | GWYQQKPGKAPKALIYSASFRYSGVPSRFSGSGSGTDFTLTISSLQPEDFATYYC |
| | | QQYYTYPYTFGGGTKVEIKSGGPGPAGMKGLPGSaptssstkktqlqlehllldlqmilnginnyk |
| | | npkltrmlafympkkatelkhlqcleeelkpleevlnlaqsknfhlrprdlisninvivlelkgsettfmceyadetativefl |
| | | nrwitfcqsiistltHHHHHH |
| 139 | ACP95 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSgsgsgsgsgsgsgsgsEVQLVESGGGLVQPGNSLRLSC |
| | protein | AASGFTFSKFGMSWVRQAPGKGLEWVSSISGSGRDTLYAESVKGRFTISRDNAK |
| | | TTLYLQMNSLRPEDTAVYYCTIGGSLSVSSQGTLVTVSSSGGPGPAGMKGLPGSa |
| | | ptssstkktqlqlehllldlqmilnginnyknpkltrmltfkfympkkatelkhlqcleeelkpleevlnlaqsknfhlrprdlis |
| | | ninvivlelkgsettfmceyadetativeflnrwitfcqsiistltHHHHHH |
| 140 | ACP96 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSSGGPGPAGMKGLPGSaptssstkktqlqlehllldlqmilngin |
| | protein | nyknpkltrmltfkfympkkatelkhlqcleeelkpleevlnlaqsknfhlrprdlisninvivlelkgsettfmceyadetati |
| | | veflnrwitfcqsiistltSGGPGPAGMKGLPGSEVQLVESGGGLVQPGNSLRLSCAASGFT |
| | | FSKFGMSWVRQAPGKGLEWVSSISGSGRDTLYAESVKGRFTISRDNAKTTLYLQ |
| | | MNSLRPEDTAVYYCTIGGSLSVSSQGTLVTVSSHHHHHH |
| 141 | ACP97 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSggggsggggsggggsEVQLVESGGGLVQPGNSLRLSC |
| | protein | AASGFTFSKFGMSWVRQAPGKGLEWVSSISGSGRDTLYAESVKGRFTISRDNAK |
| | | TTLYLQMNSLRPEDTAVYYCTIGGSLSVSSQGTLVTVSSSGGPGPAGMKGLPGSa |
| | | ptssstkktqlqlehllldlqmilnginnyknpkltrmltfkfympkkatelkhlqcleeelkpleevlnlaqsknfhlrprdlis |
| | | ninvivlelkgsettmceyadetativeflnrwitfcqsiistltSGGPGPAGMKGLPGSEVQLVESGGGL |
| | | VQPGNSLRLSCAASGFTFSKFGMSWVRQAPGKGLEWVSSISGSGRDTLYAESVK |
| | | GRFTISRDNAKTTLYLQMNSLRPEDTAVYYCTIGGSLSVSSQGTLVTVSSHHHHH |
| | | H |
| 142 | ACP99 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSggggsggggsggggsaptssstkktqlqlehllldlqmilnginnyknpk |
| | protein | ltrmltfkfympkkatelkhlqcleeelkpleevlnlaqsknfhlrprdlisninvivlelkgsettfmceyadetativeflnrw |
| | | itfcqsiistltSGGPGPAGMKGLPGSEVQLVESGGGLVQPGNSLRLSCAASGFTFSKFG |
| | | MSWVRQAPGKGLEWVSSISGSGRDTLYAESVKGRFTISRDNAKTTLYLQMNSLR |
| | | PEDTAVYYCTIGGSLSVSSQGTLVTVSSHHHHHH |
| 143 | ACP100 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSggggsggggsggggsaptssstkktqlqlehllldlqmilnginnyknpk |
| | protein | ltrmltfkfympkkatelkhlqcleeelkpleevlnlaqsknfhlrprdlisninvivlelkgsettfmceyadetativeflnrw |
| | | tfcqsiistltHHHHHH |
| 144 | ACP101 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSEVQLVESGGGLVQPGNSLRLSCAASGFTFSKFGMSWVRQAPGKGLEWV |
| | protein | SSISGSGRDTLYAESVKGRFTISRDNAKTTLYLQMNSLRPEDTAVYYCTIGGSLS |
| | | VSSQGTLVTVSSHHHHHH |
| 145 | ACP102 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSSGGPGPAGMKGLPGSaptssstkktqlqlehllldlqmilngin |
| | protein | nyknpkltrmltfkfympkkatelkhlqcleeelkpleevlnlaqsknfhlrprdlisninvivlelkgsettfmceyadetati |
| | | veflnrwitfcqsiistltSGGPGPAGMKGLPGSEVQLVESGGGLVQPGNSLRLSCAASGFT |
| | | FSKFGMSWVRQAPGKGLEWVSSISGSGRDTLYAESVKGRFTISRDNAKTTLYLQ |
| | | MNSLRPEDTAVYYCTIGGSLSVSSQGTLVTVSSggggsggggsggggsggggsggggsggggs |
| | | EVQLVESGGGLVQPGGSLRLSCAASGFTFSSYTLAWVRQAPGKGLEWVAAIDSS |
| | | SYTYSPDTVRGRFTISRDNAKNSLYLQMNSLRAEDTAVYYCARDSNWDALDYW |
| | | GQGTTVTVSSGGGGSGGGGSGGGGSDIQMTQSPSSLSASVGDRVTITCKASQNV |
| | | GTNVGWYQQKPGKAPKALIYSASFRYSGVPSRFSGSGSGTDFTLTISSLQPEDFA |
| | | TYYCQQYYTYPYTFGGGTKVEIKHHHHHH |
| 146 | ACP103 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSggggsggggsggggsggggsggggsggggsEVQLVESGGGLVQPGGSLRLSCAASGF |
| | protein | TFSSYTLAWVRQAPGKGLEWVAAIDSSSYTYSPDTVRGRFTISRDNAKNSLYLQ |
| | | MNSLRAEDTAVYYCARDSNWDALDYWGQGTTVTVSSGGGGSGGGGSGGGGS |
| | | DIQMTQSPSSLSASVGDRVTITCKASQNVGTNVGWYQQKPGKAPKALIYSASFR |
| | | YSGVPSRFSGSGSGTDFTLTISSLQPEDFATYYCQQYYTYPYTFGGGTKVEIKggg |
| | | gsggggsggggsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsQVQLQESGGGLAQAGGSLSLSCAASGFTV |
| | | SNSVMAWYRQTPGKQREFVAIINSVGSTNYADSVKGRFTISRDNAKNTVYLQM |
| | | NNLKPEDTAVYVCNRNFDRIYWGQGTQVTVSSHHHHHH |
| 147 | ACP104 | mdmrvpaqllgllllwlrgarcQVQLQESGGGLAQAGGSLSLSCAASGFTVSNSVMAWYR |
| | IL-2 | QTPGKQREFVAIINSVGSTNYADSVKGRFTISRDNAKNTVYLQMNNLKPEDTAV |
| | fusion | YVCNRNFDRIYWGQGTQVTVSSaptssstkktqlqlehllldlqmilnginnyknpkltrmltfkfympkk |
| | protein | atelkhlqcleeelkpleevlnlaqsknfhlrprdlisninvivlelkgsettfmceyadetativeflnrwitfcqsiistltSGG |
| | | PGPAGMKGLPGSEVQLVESGGGLVQPGNSLRLSCAASGFTFSKFGMSWVRQAP |
| | | GKGLEWVSSISGSGRDTLYAESVKGRFTISRDNAKTTLYLQMNSLRPEDTAVYY |
| | | CTIGGSLSVSSQGTLVTVSSggggsggggsggggsggggsggggsggggsEVQLVESGGGLVQ |
| | | PGGSLRLSCAASGFTFSSYTLAWVRQAPGKGLEWVAAIDSSSYTYSPDTVRGRF |
| | | TISRDNAKNSLYLQMNSLRAEDTAVYYCARDSNWDALDYWGQGTTVTVSSGG |
| | | GGSGGGGSGGGGSDIQMTQSPSSLSASVGDRVTITCKASQNVGTNVGWYQQKP |
| | | GKAPKALIYSASFRYSGVPSRFSGSGSGTDFTLTISSLQPEDFATYYCQQYYTYPY |
| | | TFGGGTKVEIKHHHHHH |
| 148 | ACP105 | mdmrvpaqllgllllwlrgarcEVQLVESGGGLVQPGGSLRLSCAASGFTFSSYTLAWVRQ |
| | IL-2 | APGKGLEWVAAIDSSSYTYSPDTVRGRFTISRDNAKNSLYLQMNSLRAEDTAVY |
| | fusion | YCARDSNWDALDYWGQGTTVTVSSGGGGSGGGGSGGGGSDIQMTQSPSSLSAS |
| | protein | VGDRVTITCKASQNVGTNVGWYQQKPGKAPKALIYSASFRYSGVPSRFSGSGSG |
| | | TDFTLTISSLQPEDFATYYCQQYYTYPYTFGGGTKVEIKggggsggggsggggsggggsgg |
| | | ggsggggsSGGPGPAGMKGLPGSaptssstkktqlqlehllldlqmilnginnyknpkltrmltfkfympkkate |
| | | lkhlqcleeelkpleevlnlaqsknfhlrprdlisninvivlelkgsettfmceyadetativeflnrwitfcqsiistltSGGPG |
| | | PAGMKGLPGSEVQLVESGGGLVQPGNSLRLSCAASGFTFSKFGMSWVRQAPGK |
| | | GLEWVSSISGSGRDTLYAESVKGRFTISRDNAKTTLYLQMNSLRPEDTAVYYCTI |
| | | GGSLSVSSQGTLVTVSSggggsggggsggggsQVQLQESGGGLAQAGGSLSLSCAASG |
| | | FTVSNSVMAWYRQTPGKQREFVAIINSVGSTNYADSVKGRFTISRDNAKNTVYL |
| | | QMNNLKPEDTAVYVCNRNFDRIYWGQGTQVTVSSHHHHHH |
| 149 | ACP106 | mdmrvpaqllgllllwlrgarcQVQLQESGGGLAQAGGSLSLSCAASGFTVSNSVMAWYR |
| | IL-2 | QTPGKQREFVAIINSVGSTNYADSVKGRFTISRDNAKNTVYLQMNNLKPEDTAV |
| | fusion | YVCNRNFDRIYWGQGTQVTVSSggggsggggsggggsEVQLVESGGGLVQPGNSLRLS |
| | protein | CAASGFTFSKFGMSWVRQAPGKGLEWVSSISGSGRDTLYAESVKGRFTISRDNA |
| | | KTTLYLQMNSLRPEDTAVYYCTIGGSLSVSSQGTLVTVSSSGGPGPAGMKGLPG |
| | | SEVQLVESGGGLVQPGGSLRLSCAASGFTFSSYTLAWVRQAPGKGLEWVAAIDS |
| | | SSYTYSPDTVRGRFTISRDNAKNSLYLQMNSLRAEDTAVYYCARDSNWDALDY |
| | | WGQGTTVTVSSGGGGSGGGGSGGGGSDIQMTQSPSSLSASVGDRVTITCKASQN |
| | | VGTNVGWYQQKPGKAPKALIYSASFRYSGVPSRFSGSGSGTDFTLTISSLQPEDF |
| | | ATYYCQQYYTYPYTFGGGTKVEIKggggsggggsggggsggggsggggsggggsSGGPGPAG |
| | | MKGLPGSaptssstkktqlqlehllldlqmilnginnyknpkltrmltfkfympkkatelkhlqcleeelkpleevlnlaq |
| | | sknfhlrprdlisninvivlelkgsettfmceyadetativeflnrwitfcqsiistltHHHHHH |
| 150 | ACP107 | mdmrvpaqllgllllwlrgarcEVQLVESGGGLVQPGGSLRLSCAASGFTFSSYTLAWVRQ |
| | IL-2 | APGKGLEWVAAIDSSSYTYSPDTVRGRFTISRDNAKNSLYLQMNSLRAEDTAVY |
| | fusion | YCARDSNWDALDYWGQGTTVTVSSGGGGSGGGGSGGGGSDIQMTQSPSSLSAS |
| | protein | VGDRVTITCKASQNVGTNVGWYQQKPGKAPKALIYSASFRYSGVPSRFSGSGSG |
| | | TDFTLTISSLQPEDFATYYCQQYYTYPYTFGGGTKVEIKggggsggggsggggsggggsgg |
| | | ggsggggsEVQLVESGGGLVQPGNSLRLSCAASGFTFSKFGMSWVRQAPGKGLEW |
| | | VSSISGSGRDTLYAESVKGRFTISRDNAKTTLYLQMNSLRPEDTAVYYCTIGGSL |
| | | SVSSQGTLVTVSSSGGPGPAGMKGLPGSaptssstkktqlqlehllldlqmilnginnyknpkltrmltf |
| | | kfympkkatelkhlqcleeelkpleevlnlaqsknfhlrprdlisninvivlelkgsettfmceyadetativeflnrwitfcqsi |
| | | istltggggsggggsggggsQVQLQESGGGLAQAGGSLSLSCAASGFTVSNSVMAWYRQT |
| | | PGKQREFVAIINSVGSTNYADSVKGRFTISRDNAKNTVYLQMNNLKPEDTAVYV |
| | | CNRNFDRIYWGQGTQVTVSSHHHHHH |
| 151 | ACP108 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSggggsggggsggggsaptssstkktqlqlehllldlqmilnginnyknpk |
| | protein | ltrmltfkfympkkatelkhlqcleeelkpleevlnlaqsknfhlrprdlisninvivlelkgsettfmceyadetativeflnrw |
| | | itfcqsiistltSGGPGPAGMKGLPGSrgetgpaaPGSEVQLVESGGGLVQPGNSLRLSCAAS |
| | | GFTFSKFGMSWVRQAPGKGLEWVSSISGSGRDTLYAESVKGRFTISRDNAKTTL |
| | | YLQMNSLRPEDTAVYYCTIGGSLSVSSQGTLVTVSSggggsggggsggggsggggsggggsg |
| | | gggsEVQLVESGGGLVQPGGSLRLSCAASGFTFSSYTLAWVRQAPGKGLEWVAAI |
| | | DSSSYTYSPDTVRGRFTISRDNAKNSLYLQMNSLRAEDTAVYYCARDSNWDAL |
| | | DYWGQGTTVTVSSGGGGSGGGGSGGGGSDIQMTQSPSSLSASVGDRVTITCKAS |
| | | QNVGTNVGWYQQKPGKAPKALIYSASFRYSGVPSRFSGSGSGTDFTLTISSLQPE |
| | | DFATYYCQQYYTYPYTFGGGTKVEIKHHHHHH |
| 152 | ACP117 | mdmrvpaqllgllllwlrgarcEVQLVESGGGLVQPGGSLRLSCAASGFTFSSYAMSWVRQ |
| | Anti-FN | APGKGLEWVSAISGSGGSTYYADSVKGRFTISRDNSKNTLYLQMNSLRAEDTAV |
| | CGS-2 | YYCARGVGAFRPYRKHEWGQGTLVTVSRggggsggggsggggsSSELTQDPAVSVAL |
| | scFv | GQTVRITCQGDSLRSYYASWYQQKPGQAPVLVIYGKNNRPSGIPDRFSGSSSGNT |
| | | ASLTTTGAQAEDEADYYCNSSPFEHNLVVFGGGTKLTVLHHHHHHEPEA |
| 153 | ACP118 | mdmrvpaqllgllllwlrgarcQVQLQQSGAELVRPGTSVKVSCKASGYAFTNYLIEWVKQ |
| | NARA1 | RPGQGLEWIGVINPGSGGTNYNEKFKGKATLTADKSSSTAYMQLSSLTSDDSAV |
| | Vh/V1 | YFCARWRGDGYYAYFDVWGAGTTVTVSSggggsggggsggggsDIVLTQSPASLAVS |
| | non- | LGQRATISCKASQSVDYDGDSYMNWYQQKPGQPPKLLIYAASNLESGIPARFSG |
| | cleavable | SGSGTDFTLNIHPVEEEDAATYYCQQSNEDPYTFGGGTKLEIKHHHHHHEPEA |
| 154 | ACP119 | mdmrvpaqllgllllwlrgarcQVQLQQSGAELVRPGTSVKVSCKASGYAFTNYLIEWVKQ |
| | NARA1 | RPGQGLEWIGVINPGSGGTNYNEKFKGKATLTADKSSSTAYMQLSSLTSDDSAV |
| | Vh/V1 | YFCARWRGDGYYAYFDVWGAGTTVTVSSSGGPGPAGMKGLPGSDIVLTQSPAS |
| | cleavable | LAVSLGQRATISCKASQSVDYDGDSYMNWYQQKPGQPPKLLIYAASNLESGIPA |
| | | RFSGSGSGTDFTLNIHPVEEEDAATYYCQQSNEDPYTFGGGTKLEIKHHHHHHEP |
| | | EA |
| 155 | ACP120 | mdmrvpaqllgllllwlrgarcDIVLTQSPASLAVSLGQRATISCKASQSVDYDGDSYMNW |
| | NARA1 | YQQKPGQPPKLLIYAASNLESGIPARFSGSGSGTDFTLNIHPVEEEDAATYYCQQS |
| | V1/Vh | NEDPYTFGGGTKLEIKggggsggggsggggsQVQLQQSGAELVRPGTSVKVSCKASGY |
| | non- | AFTNYLIEWVKQRPGQGLEWIGVINPGSGGTNYNEKFKGKATLTADKSSSTAYM |
| | cleavable | QLSSLTSDDSAVYFCARWRGDGYYAYFDVWGAGTTVTVSSHHHHHHEPEA |
| 156 | ACP121 | mdmrvpaqllgllllwlrgarcDIVLTQSPASLAVSLGQRATISCKASQSVDYDGDSYMNW |
| | NARA1 | YQQKPGQPPKLLIYAASNLESGIPARFSGSGSGTDFTLNIHPVEEEDAATYYCQQS |
| | V1/Vh | NEDPYTFGGGTKLEIKSGGPGPAGMKGLPGSQVQLQQSGAELVRPGTSVKVSCK |
| | cleavable | ASGYAFTNYLIEWVKQRPGQGLEWIGVINPGSGGTNYNEKFKGKATLTADKSSS |
| | | TAYMQLSSLTSDDSAVYFCARWRGDGYYAYFDVWGAGTTVTVSSHHHHHHEP |
| | | EA |
| 157 | ACP124 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltggggsggggsggggs |
| | fusion | EVQLVESGGGLVQPGNSLRLSCAASGFTFSKFGMSWVRQAPGKGLEWVSSISGS |
| | protein | GRDTLYAESVKGRFTISRDNAKTTLYLQMNSLRPEDTAVYYCTIGGSLSVSSQGT |
| | | LVTVSSHHHHHHEPEA |
| 158 | ACP132 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltggggsggggsggggs |
| | fusion | dahksevahrfkdlgeenfkalvliafaqylqqcpfedhvklvnevtefaktcvadesaencdkslhtlfgdklctvatlrety |
| | protein | gemadccakqepernecflqhkddnpnlprlvrpevdvmctafhdneetflkkylyeiarrhpyfyapellffakrykaaft |
| | | eccqaadkaacllpkldelrdegkassakqrlkcaslqkfgerafkawavarlsqrfpkaefaevsklvtdltkvhtecchgdl |
| | | lecaddradlakyicenqdsissklkeccekpllekshciaevendempadlpslaadfveskdvcknyaeakdvflgmfl |
| | | yeyarrhpdysvvlllrlaktyettlekccaaadphecyakvfdefkplveepqnlikqncelfeqlgeykfqnallvrytkkv |
| | | pqvstptlvevsrnlgkvgskcckhpeakrmpcaedylsvvlnqlcvlhektpvsdrvtkccteslvnrrpcfsalevdety |
| | | vpkefnaetftfhadictlsekerqikkqtalvelvkhkpkatkeqlkavmddfaafvekcckaddketcfaeegkklvaas |
| | | qaalglHHHHHHEPEA |
| 159 | ACP141 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltggggsggggsggggs |
| | fusion | dahksevahrfkdlgeenfkalvliafaqylqqcpfedhvklvnevtefaktcvadesaencdkslhtlfgdklctvatlrety |
| | protein | gemadccakqepernecflqhkddnpnlprlvrpevdvmctafhdneetflkkylyeiarrhpyfyapellffakrykaaft |
| | | eccqaadkaacllpkldelrdegkassakqrlkcaslqkfgerafkawavarlsqrfpkaefaevsklvtdltkvhtecchgdl |
| | | lecaddradlakyicenqdsissklkeccekpllekshciaevendempadlpslaadfveskdvcknyaeakdvflgmfl |
| | | yeyarrhpdysvvlllrlaktyettlekccaaadphecyakvfdefkplveepqnlikqncelfeqlgeykfqnallvrytkkv |
| | | pqvstptlvevsrnlgkvgskcckhpeakrmpcaedylsvvlnqlcvlhektpvsdrvtkccteslvnrrpcfsalevdety |
| | | vpkefnaetftfhadictlsekerqikkqtalvelvkhkpkatkeqlkavmddfaafvekcckaddketcfaeegkklvaas |
| | | qaalglHHHHHHEPEA |
| 160 | ACP142 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSdahksevahrfkdlgeenfkalvliafaqylqqcpfedhvklvnevtefaktcvadesaencdkslhtlfgdklct |
| | protein | vatlretygemadccakqepernecflqhkddnpnlprlvrpevdvmctafhdneetflkkylyeiarrhpyfyapellffa |
| | | krykaafteccqaadkaacllpkldelrdegkassakqrlkcaslqkfgerafkawavarlsqrfpkaefaevsklvtdltkvh |
| | | tecchgdllecaddradlakyicenqdsissklkeccekpllekshciaevendempadlpslaadfveskdvcknyaeak |
| | | dvflgmflyeyarrhpdysvvlllrlaktyettlekccaaadphecyakvfdefkplveepqnlikqncelfeqlgeykfqna |
| | | llvrytkkvpqvstptlvevsrnlgkvgskcckhpeakrmpcaedylsvvlnqlcvlhektpvsdrvtkccteslvnrrpcfs |
| | | alevdetyvpkefnaetftfhadictlsekerqikkqtalvelvkhkpkatkeqlkavmddfaafvekcckaddketcfaee |
| | | gkklvaasqaalglHHHHHHEPEA |
| 161 | ACP144 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSEVQLVESGGGLVQPGNSLRLSCAASGFTFSKFGMSWVRQAPGKGLEWV |
| | protein | SSISGSGRDTLYAESVKGRFTISRDNAKTTLYLQMNSLRPEDTAVYYCTIGGSLS |
| | | VSSQGTLVTVSSggggsggggsggggsggggsggggsggggsSGGPGPAGMKGLPGSEVQLV |
| | | ESGGGLVQPGGSLRLSCAASGFTFSSYTLAWVRQAPGKGLEWVAAIDSSSYTYS |
| | | PDTVRGRFTISRDNAKNSLYLQMNSLRAEDTAVYYCARDSNWDALDYWGQGT |
| | | TVTVSSGGGGSGGGGSGGGGSDIQMTQSPSSLSASVGDRVTITCKASQNVGTNV |
| | | GWYQQKPGKAPKALIYSASFRYSGVPSRFSGSGSGTDFTLTISSLQPEDFATYYC |
| | | QQYYTYPYTFGGGTKVEIKggggsggggsggggsQVQLQESGGGLAQAGGSLSLSCAA |
| | | SGFTVSNSVMAWYRQTPGKQREFVAIINSVGSTNYADSVKGRFTISRDNAKNTV |
| | | YLQMNNLKPEDTAVYVCNRNFDRIYWGQGTQVTVSSHHHHHHEPEA |
| 162 | ACP145 | mdmrvpaqllgllllwlrgarcQVQLQESGGGLAQAGGSLSLSCAASGFTVSNSVMAWYR |
| | IL-2 | QTPGKQREFVAIINSVGSTNYADSVKGRFTISRDNAKNTVYLQMNNLKPEDTAV |
| | fusion | YVCNRNFDRIYWGQGTQVTVSSggggsggggsggggsaptssstkktqlqlehllldlqmilnginnykn |
| | protein | pkltrmltfkfympkkatelkhlqcleeelkpleevlnlaqsknfhlrprdlisninvivlelkgsettfmceyadetativefln |
| | | rwitfcqsiistltSGGPGPAGMKGLPGSEVQLVESGGGLVQPGNSLRLSCAASGFTFSK |
| | | FGMSWVRQAPGKGLEWVSSISGSGRDTLYAESVKGRFTISRDNAKTTLYLQMNS |
| | | LRPEDTAVYYCTIGGSLSVSSQGTLVTVSSggggsggggsggggsggggsggggsggggsSGG |
| | | PGPAGMKGLPGSEVQLVESGGGLVQPGGSLRLSCAASGFTFSSYTLAWVRQAPG |
| | | KGLEWVAAIDSSSYTYSPDTVRGRFTISRDNAKNSLYLQMNSLRAEDTAVYYCA |
| | | RDSNWDALDYWGQGTTVTVSSGGGGSGGGGSGGGGSDIQMTQSPSSLSASVGD |
| | | RVTITCKASQNVGTNVGWYQQKPGKAPKALIYSASFRYSGVPSRFSGSGSGTDF |
| | | TLTISSLQPEDFATYYCQQYYTYPYTFGGGTKVEIKHHHHHHEPEA |
| 163 | ACP146 | mdmrvpaqllgllllwlrgarcQVQLQESGGGLAQAGGSLSLSCAASGFTVSNSVMAWYR |
| | IL-2 | QTPGKQREFVAIINSVGSTNYADSVKGRFTISRDNAKNTVYLQMNNLKPEDTAV |
| | fusion | YVCNRNFDRIYWGQGTQVTVSSSGGPGPAGMKGLPGSaptssstkktqlqlehllldlqmilng |
| | protein | innyknpkltrmltfkfympkkatelkhlqcleeelkpleevlnlaqsknfhlrprdlisninvivlelkgsettfmceyadeta |
| | | tiveflnrwitfcqsiistltSGGPGPAGMKGLPGSEVQLVESGGGLVQPGNSLRLSCAASGF |
| | | TFSKFGMSWVRQAPGKGLEWVSSISGSGRDTLYAESVKGRFTISRDNAKTTLYL |
| | | QMNSLRPEDTAVYYCTIGGSLSVSSQGTLVTVSSggggsggggsggggsggggsggggsggg |
| | | gsSGGPGPAGMKGLPGSEVQLVESGGGLVQPGGSLRLSCAASGFTFSSYTLAWV |
| | | RQAPGKGLEWVAAIDSSSYTYSPDTVRGRFTISRDNAKNSLYLQMNSLRAEDTA |
| | | VYYCARDSNWDALDYWGQGTTVTVSSGGGGSGGGGSGGGGSDIQMTQSPSSL |
| | | SASVGDRVTITCKASQNVGTNVGWYQQKPGKAPKALIYSASFRYSGVPSRFSGS |
| | | GSGTDFTLTISSLQPEDFATYYCQQYYTYPYTFGGGTKVEIKHHHHHHEPEA |
| 164 | ACP133 | mdmrvpaqllgllllwlrgarcaptssstkktqlqlehllldlqmilnginnyknpkltrmltfkfympkkatelkhlqcleee |
| | IL-2- | lkpleevlnlaqsknfhlrprdlisninvivlelkgsettfmceyadetativeflnrwitfcqsiistltHHHHHH |
| | 6xHis | |
| | (“6xHis” | |
| | disclosed | |
| | as SEQ | |
| | ID NO.: | |
| | 354) | |
| 165 | ACP147 | mdmrvpaqllgllllwlrgarcaptssstkktqlqlehllldlqmilnginnyknpkltrmltfkfympkkatelkhlqcleee |
| | IL-2 | lkpleevlnlaqsknfhlrprdlisninvivlelkgsettfmceyadetativeflnrwitfcqsiistltSGGPGPAGMK |
| | fusion | GLPGSEVQLVESGGGLVQPGNSLRLSCAASGFTFSKFGMSWVRQAPGKGLEWV |
| | protein | SSISGSGRDTLYAESVKGRFTISRDNAKTTLYLQMNSLRPEDTAVYYCTIGGSLS |
| | | VSSQGTLVTVSSggggsggggsggggsggggsggggsggggsSGGPGPAGMKGLPGSEVQLV |
| | | ESGGGLVQPGGSLRLSCAASGFTFSSYTLAWVRQAPGKGLEWVAAIDSSSYTYS |
| | | PDTVRGRFTISRDNAKNSLYLQMNSLRAEDTAVYYCARDSNWDALDYWGQGT |
| | | TVTVSSGGGGSGGGGSGGGGSDIQMTQSPSSLSASVGDRVTITCKASQNVGTNV |
| | | GWYQQKPGKAPKALIYSASFRYSGVPSRFSGSGSGTDFTLTISSLQPEDFATYYC |
| | | QQYYTYPYTFGGGTKVEIKggggsggggsggggsQVQLQESGGGLVQAGGSLRLSCA |
| | | ASGRIFSIDIMSWYRQAPGKQRELVARITRGGTISYDDSVKGRFTISRDNAKNTV |
| | | YLQMNSLKPEDTGVYYCNALYGTDYWGKGTQVTVSSHHHHHHEPEA |
| 166 | ACP148 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSggggsggggsggggsaptssstkktqlqlehllldlqmilnginnyknpk |
| | protein | ltrmltfkfympkkatelkhlqcleeelkpleevlnlaqsknfhlrprdlisninvivlelkgsettfmceyadetativeflnrw |
| | | itfcqsiistltSGGPGPAGMKGLPGSEVQLVESGGGLVQPGNSLRLSCAASGFTFSKFG |
| | | MSWVRQAPGKGLEWVSSISGSGRDTLYAESVKGRFTISRDNAKTTLYLQMNSLR |
| | | PEDTAVYYCTIGGSLSVSSQGTLVTVSSggggsggggsggggsggggsggggsggggsSGGPG |
| | | PAGMKGLPGSEVQLVESGGGLVQPGGSLRLSCAASGFTFSSYTLAWVRQAPGK |
| | | GLEWVAAIDSSSYTYSPDTVRGRFTISRDNAKNSLYLQMNSLRAEDTAVYYCAR |
| | | DSNWDALDYWGQGTTVTVSSGGGGSGGGGSGGGGSDIQMTQSPSSLSASVGDR |
| | | VTITCKASQNVGTNVGWYQQKPGKAPKALIYSASFRYSGVPSRFSGSGSGTDFT |
| | | LTISSLQPEDFATYYCQQYYTYPYTFGGGTKVEIKHHHHHHEPEA |
| 167 | ACP149 | mdmrvpaqllgllllwlrgarcQVQLQESGGGLVQAGGSLRLSCAASGRIFSIDIMSWYRQA |
| | IL-2 | PGKQRELVARITRGGTISYDDSVKGRFTISRDNAKNTVYLQMNSLKPEDTGVYY |
| | fusion | CNALYGTDYWGKGTQVTVSSSGGPGPAGMKGLPGSaptssstkktqlqlehllldlqmilngin |
| | protein | nyknpkltrmltfkfympkkatelkhlqcleeelkpleevlnlaqsknfhlrprdlisninvivlelkgsettfmceyadetati |
| | | veflnrwitfcqsiistltSGGPGPAGMKGLPGSEVQLVESGGGLVQPGNSLRLSCAASGFT |
| | | FSKFGMSWVRQAPGKGLEWVSSISGSGRDTLYAESVKGRFTISRDNAKTTLYLQ |
| | | MNSLRPEDTAVYYCTIGGSLSVSSQGTLVTVSSggggsggggsggggsggggsggggsggggs |
| | | SGGPGPAGMKGLPGSEVQLVESGGGLVQPGGSLRLSCAASGFTFSSYTLAWVRQ |
| | | APGKGLEWVAAIDSSSYTYSPDTVRGRFTISRDNAKNSLYLQMNSLRAEDTAVY |
| | | YCARDSNWDALDYWGQGTTVTVSSGGGGSGGGGSGGGGSDIQMTQSPSSLSAS |
| | | VGDRVTITCKASQNVGTNVGWYQQKPGKAPKALIYSASFRYSGVPSRFSGSGSG |
| | | TDFTLTISSLQPEDFATYYCQQYYTYPYTFGGGTKVEIKHHHHHHEPEA |
| 168 | ACP33 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFNa- | YYCTIGGSLSVSSQGTLVTVSSSGGPGPAGMKGLPGScdlpqthnlrnkraltllvqmrrlsplsc |
| | fusion | lkdrkdfgfpqekvdaqqikkapipvlseltqqilniftskdssaawnttlldsfcndlhqqlndlqgclmqqvgvqefplt |
| | protein | qedallavrkyfhritvylrekkhspcawevvraevwralsssanvSGGPGPAGMKGLPGSEVQLVESG |
| | | GGLVQPGNSLRLSCAASGFTFSKFGMSWVRQAPGKGLEWVSSISGSGRDTLYAE |
| | | SVKGRFTISRDNAKTTLYLQMNSLRPEDTAVYYCTIGGSLSVSSQGTLVTVSSHH |
| | | HHHHEPEA |
| 169 | ACP131 | mdmrvpaqllgllllwlrgarccdlpqthnlrnkraltllvqmrrlsplsclkdrkdfgfpqekvdaqqikkaqaipvlseltq |
| | Mouse | qilniftskdssaawnttlldsfcndlhqqlndlqgclmqqvgvqefpltqedallavrkyfhritvylrekkhspcawevvr |
| | IFNa | aevwralsssanvlgrlreekHHHHHHEPEA |
| 170 | ACP125 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFNa- | YYCTIGGSLSVSSQGTLVTVSSSGGPGPAGMKGLPGScdlpqthnlrnkraltllvqmrrlsplsc |
| | fusion | lkdrkdfgfpqekvdaqqikkaqaipvlseltqqilniftskdssaawnttlldsfcndlhqqlndlqgclmqqvgvqefplt |
| | protein | qedallavrkyfhritvylrekkhspcawevvraevwralsssanvlgrlreekHHHHHHEPEA |
| 171 | ACP126 | mdmrvpaqllgllllwlrgarccdlpqthnlrnkraltllvqmrrlsplsclkdrkdfgfpqekvdaqqikkaqaipvlseltq |
| | Mouse | qilniftskdssaawnttlldsfcndlhqqlndlqgclmqqvgvqefpltqedallavrkyfhritvylrekkhspcawevvr |
| | IFNa- | aevwralsssanvlgrlreekSGGPGPAGMKGLPGSEVQLVESGGGLVQPGNSLRLSCAAS |
| | fusion | GFTFSKFGMSWVRQAPGKGLEWVSSISGSGRDTLYAESVKGRFTISRDNAKTTL |
| | protein | YLQMNSLRPEDTAVYYCTIGGSLSVSSQGTLVTVSSHHHHHHEPEA |
| 172 | ACP127 | mdmrvpaqllgllllwlrgarcEAHKSEIAHRYNDLGEQHFKGLVLIAFSQYLQKCSYDEHA |
| | Mouse | KLVQEVTDFAKTCVADESAANCDKSLHTLFGDKLCAIPNLRENYGELADCCTK |
| | IFNa- | QEPERNECFLQHKDDNPSLPPFERPEAEAMCTSFKENPTTFMGHYLHEVARRHP |
| | fusion | YFYAPELLYYAEQYNEILTQCCAEADKESCLTPKLDGVKEKALVSSVRQRMKCS |
| | protein | SMQKFGERAFKAWAVARLSQTFPNADFAEITKLATDLTKVNKECCHGDLLECA |
| | | DDRAELAKYMCENQATISSKLQTCCDKPLLKKAHCLSEVEHDTMPADLPAIAAD |
| | | FVEDQEVCKNYAEAKDVFLGTFLYEYSRRHPDYSVSLLLRLAKKYEATLEKCCA |
| | | EANPPACYGTVLAEFQPLVEEPKNLVKTNCDLYEKLGEYGFQNAILVRYTQKAP |
| | | QVSTPTLVEAARNLGRVGTKCCTLPEDQRLPCVEDYLSAILNRVCLLHEKTPVSE |
| | | HVTKCCSGSLVERRPCFSALTVDETYVPKEFKAETFTFHSDICTLPEKEKQIKKQT |
| | | ALAELVKHKPKATAEQLKTVMDDFAQFLDTCCKAADKDTCFSTEGPNLVTRCK |
| | | DALASGGPGPAGMKGLPGScdlpqthnlrnkraltllvqmrrlsplsclkdrkdfgfpqekvdaqqikkaqai |
| | | pvlseltqqilniftskdssaawnttlldsfcndlhqqlndlqgclmqqvgvqefpltqedallavrkyfhritvylrekkhspc |
| | | awevvraevwralsssanvlgrlreekSGGPGPAGMKGLPGSEAHKSEIAHRYNDLGEQHFKG |
| | | LVLIAFSQYLQKCSYDEHAKLVQEVTDFAKTCVADESAANCDKSLHTLFGDKLC |
| | | AIPNLRENYGELADCCTKQEPERNECFLQHKDDNPSLPPFERPEAEAMCTSFKEN |
| | | PTTFMGHYLHEVARRHPYFYAPELLYYAEQYNEILTQCCAEADKESCLTPKLDG |
| | | VKEKALVSSVRQRMKCSSMQKFGERAFKAWAVARLSQTFPNADFAEITKLATD |
| | | LTKVNKECCHGDLLECADDRAELAKYMCENQATISSKLQTCCDKPLLKKAHCL |
| | | SEVEHDTMPADLPAIAADFVEDQEVCKNYAEAKDVFLGTFLYEYSRRHPDYSVS |
| | | LLLRLAKKYEATLEKCCAEANPPACYGTVLAEFQPLVEEPKNLVKTNCDLYEKL |
| | | GEYGFQNAILVRYTQKAPQVSTPTLVEAARNLGRVGTKCCTLPEDQRLPCVEDY |
| | | LSAILNRVCLLHEKTPVSEHVTKCCSGSLVERRPCFSALTVDETYVPKEFKAETFT |
| | | FHSDICTLPEKEKQIKKQTALAELVKHKPKATAEQLKTVMDDFAQFLDTCCKAA |
| | | DKDTCFSTEGPNLVTRCKDALAHHHHHHEPEA |
| 173 | ACP128 | mdmrvpaqllgllllwlrgarcEAHKSEIAHRYNDLGEQHFKGLVLIAFSQYLQKCSYDEHA |
| | Mouse | KLVQEVTDFAKTCVADESAANCDKSLHTLFGDKLCAIPNLRENYGELADCCTK |
| | IFNa- | QEPERNECFLQHKDDNPSLPPFERPEAEAMCTSFKENPTTFMGHYLHEVARRHP |
| | fusion | YFYAPELLYYAEQYNEILTQCCAEADKESCLTPKLDGVKEKALVSSVRQRMKCS |
| | protein | SMQKFGERAFKAWAVARLSQTFPNADFAEITKLATDLTKVNKECCHGDLLECA |
| | | DDRAELAKYMCENQATISSKLQTCCDKPLLKKAHCLSEVEHDTMPADLPAIAAD |
| | | FVEDQEVCKNYAEAKDVFLGTFLYEYSRRHPDYSVSLLLRLAKKYEATLEKCCA |
| | | EANPPACYGTVLAEFQPLVEEPKNLVKTNCDLYEKLGEYGFQNAILVRYTQKAP |
| | | QVSTPTLVEAARNLGRVGTKCCTLPEDQRLPCVEDYLSAILNRVCLLHEKTPVSE |
| | | HVTKCCSGSLVERRPCFSALTVDETYVPKEFKAETFTFHSDICTLPEKEKQIKKQT |
| | | ALAELVKHKPKATAEQLKTVMDDFAQFLDTCCKAADKDTCFSTEGPNLVTRCK |
| | | DALASGGPGPAGMKGLPGScdlpqthnlrnkraltllvqmrrlsplsclkdrkdfgfpqekvdaqqikkaqai |
| | | pvlseltqqilniftskdssaawnttlldsfcndlhqqlndlqgclmqqvgvqefpltqedallavrkyfhritvylrekkhspc |
| | | awevvraevwralsssanvlgrlreekHHHHHHEPEA |
| 174 | ACP129 | mdmrvpaqllgllllwlrgarccdlpqthnlrnkraltllvqmrrlsplsclkdrkdfgfpqekvdaqqikkaqaipvlseltq |
| | Mouse | qilniftskdssaawnttlldsfcndlhqqlndlqgclmqqvgvqefpltqedallavrkyfhritvylrekkhspcawevvr |
| | IFNa- | aevwralsssanvlgrlreekSGGPGPAGMKGLPGSEAHKSEIAHRYNDLGEQHFKGLVLI |
| | fusion | AFSQYLQKCSYDEHAKLVQEVTDFAKTCVADESAANCDKSLHTLFGDKLCAIPN |
| | protein | LRENYGELADCCTKQEPERNECFLQHKDDNPSLPPFERPEAEAMCTSFKENPTTF |
| | | MGHYLHEVARRHPYFYAPELLYYAEQYNEILTQCCAEADKESCLTPKLDGVKE |
| | | KALVSSVRQRMKCSSMQKFGERAFKAWAVARLSQTFPNADFAEITKLATDLTK |
| | | VNKECCHGDLLECADDRAELAKYMCENQATISSKLQTCCDKPLLKKAHCLSEV |
| | | EHDTMPADLPAIAADFVEDQEVCKNYAEAKDVFLGTFLYEYSRRHPDYSVSLLL |
| | | RLAKKYEATLEKCCAEANPPACYGTVLAEFQPLVEEPKNLVKTNCDLYEKLGE |
| | | YGFQNAILVRYTQKAPQVSTPTLVEAARNLGRVGTKCCTLPEDQRLPCVEDYLS |
| | | AILNRVCLLHEKTPVSEHVTKCCSGSLVERRPCFSALTVDETYVPKEFKAETFTF |
| | | HSDICTLPEKEKQIKKQTALAELVKHKPKATAEQLKTVMDDFAQFLDTCCKAAD |
| | | KDTCFSTEGPNLVTRCKDALAHHHHHHEPEA |
| 175 | ACP150 | mdmrvpaqllgllllwlrgarcQVQLQESGGGLAQAGGSLSLSCAASGFTVSNSVMAWYR |
| | Mouse | QTPGKQREFVAIINSVGSTNYADSVKGRFTISRDNAKNTVYLQMNNLKPEDTAV |
| | IFNa- | YVCNRNFDRIYWGQGTQVTVSSggggsggggsggggsEVQLVESGGGLVQPGNSLRLS |
| | fusion | CAASGFTFSKFGMSWVRQAPGKGLEWVSSISGSGRDTLYAESVKGRFTISRDNA |
| | protein | KTTLYLQMNSLRPEDTAVYYCTIGGSLSVSSQGTLVTVSSSGGPGPAGMKGLPG |
| | | Scdlpqthnlrnkraltllvqmrrlsplsclkdrkdfgfpqekvdaqqikkapipvlseltqqilniftskdssaawnttlldsf |
| | | cndlhqqlndlqgclmqqvgvqefpltqedallavrkyfhritvylrekkhspcawevvraevwralsssanvlgrlreekS |
| | | GGPGPAGMKGLPGSEVQLVESGGGLVQPGNSLRLSCAASGFTFSKFGMSWVRQ |
| | | APGKGLEWVSSISGSGRDTLYAESVKGRFTISRDNAKTTLYLQMNSLRPEDTAV |
| | | YYCTIGGSLSVSSQGTLVTVSSHHHHHHEPEA |
| 176 | ACP151 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFNa- | YYCTIGGSLSVSSQGTLVTVSSSGGPGPAGMKGLPGScdlpqthnlrnkraltllvqmrrlsplsc |
| | fusion | lkdrkdfgfpqekvdaqqikkapipvlseltqqilniftskdssaawnttlldsfcndlhqqlndlqgclmqqvgvqefplt |
| | protein | qedallavrkyfhritvylrekkhspcawevvraevwralsssanvlgrlreekSGGPGPAGMKGLPGSEVQ |
| | | LVESGGGLVQPGNSLRLSCAASGFTFSKFGMSWVRQAPGKGLEWVSSISGSGRD |
| | | TLYAESVKGRFTISRDNAKTTLYLQMNSLRPEDTAVYYCTIGGSLSVSSQGTLVT |
| | | VSSggggsggggsggggsQVQLQESGGGLAQAGGSLSLSCAASGFTVSNSVMAWYRQ |
| | | TPGKQREFVAIINSVGSTNYADSVKGRFTISRDNAKNTVYLQMNNLKPEDTAVY |
| | | VCNRNFDRIYWGQGTQVTVSSHHHHHHEPEA |
| 177 | ACP152 | mdmrvpaqllgllllwlrgarcEVQLVESGGGLVQPGNSLRLSCAASGFTFSKFGMSWVRQ |
| | Mouse | APGKGLEWVSSISGSGRDTLYAESVKGRFTISRDNAKTTLYLQMNSLRPEDTAV |
| | IFNa- | YYCTIGGSLSVSSQGTLVTVSSggggsggggsggggscdlpqthnlrnkraltllvqmrrlsplsclkdrkdf |
| | fusion | gfpqekvdaqqikkapipvlseltqqilniftskdssaawnttlldsfcndlhqqlndlqgclmqqvgvqefpltqedalla |
| | protein | vrkyfhritvylrekkhspcawevvraevwralsssanvlgrlreekggggsggggsggggsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSHHHHHH |
| | | EPEA |
| 178 | ACP153 | mdmrvpaqllgllllwlrgarcaptssstkktqlqlehllldlqmilnginnyknpkltrmltfkfympkkatelkhlqcleee |
| | (IL-2 | lkpleevlnlaqsknfhlrprdlisninvivlelkgsettfmceyadetativeflnrwitfcqsiistltsggpGPAGLYAQ |
| | Conju- | pgsEVQLVESGGGLVQPGNSLRLSCAASGFTFSKFGMSWVRQAPGKGLEWVSSIS |
| | gate) | GSGRDTLYAESVKGRFTISRDNAKTTLYLQMNSLRPEDTAVYYCTIGGSLSVSSQ |
| | | GTLVTVSSggggsggggsggggsggggsggggsggggssggpGPAGLYAQpgsEVQLVESGGGL |
| | | VQPGGSLRLSCAASGFTFSSYTLAWVRQAPGKGLEWVAAIDSSSYTYSPDTVRG |
| | | RFTISRDNAKNSLYLQMNSLRAEDTAVYYCARDSNWDALDYWGQGTTVTVSS |
| | | GGGGSGGGGSGGGGSDIQMTQSPSSLSASVGDRVTITCKASQNVGTNVGWYQQ |
| | | KPGKAPKALIYSASFRYSGVPSRFSGSGSGTDFTLTISSLQPEDFATYYCQQYYTY |
| | | PYTFGGGTKVEIKHHHHHHEPEA |
| 179 | ACP154 | mdmrvpaqllgllllwlrgarcaptssstkktqlqlehllldlqmilnginnyknpkltrmltfkfympkkatelkhlqcleee |
| | (IL-2 | lkpleevlnlaqsknfhlrprdlisninvivlelkgsettfmceyadetativeflnrwitfcqsiistltsggpPGGPAGIGp |
| | Conju- | gsEVQLVESGGGLVQPGNSLRLSCAASGFTFSKFGMSWVRQAPGKGLEWVSSIS |
| | gate) | GSGRDTLYAESVKGRFTISRDNAKTTLYLQMNSLRPEDTAVYYCTIGGSLSVSSQ |
| | | GTLVTVSSggggsggggsggggsggggsggggsggggssggpPGGPAGIGpgsEVQLVESGGGLV |
| | | QPGGSLRLSCAASGFTFSSYTLAWVRQAPGKGLEWVAAIDSSSYTYSPDTVRGR |
| | | FTISRDNAKNSLYLQMNSLRAEDTAVYYCARDSNWDALDYWGQGTTVTVSSG |
| | | GGGSGGGGSGGGGSDIQMTQSPSSLSASVGDRVTITCKASQNVGTNVGWYQQK |
| | | PGKAPKALIYSASFRYSGVPSRFSGSGSGTDFTLTISSLQPEDFATYYCQQYYTYP |
| | | YTFGGGTKVEIKHHHHHHEPEA |
| 180 | ACP155 | mdmrvpaqllgllllwlrgarcaptssstkktqlqlehllldlqmilnginnyknpkltrmltfkfympkkatelkhlqcleee |
| | (IL-2 | lkpleevlnlaqsknfhlrprdlisninvivlelkgsettfmceyadetativeflnrwitfcqsiistltsggpALFKSSFPp |
| | Conju- | gsEVQLVESGGGLVQPGNSLRLSCAASGFTFSKFGMSWVRQAPGKGLEWVSSIS |
| | gate) | GSGRDTLYAESVKGRFTISRDNAKTTLYLQMNSLRPEDTAVYYCTIGGSLSVSSQ |
| | | GTLVTVSSggggsggggsggggsggggsggggsggggssggpALFKSSFPpgsEVQLVESGGGLV |
| | | QPGGSLRLSCAASGFTFSSYTLAWVRQAPGKGLEWVAAIDSSSYTYSPDTVRGR |
| | | FTISRDNAKNSLYLQMNSLRAEDTAVYYCARDSNWDALDYWGQGTTVTVSSG |
| | | GGGSGGGGSGGGGSDIQMTQSPSSLSASVGDRVTITCKASQNVGTNVGWYQQK |
| | | PGKAPKALIYSASFRYSGVPSRFSGSGSGTDFTLTISSLQPEDFATYYCQQYYTYP |
| | | YTFGGGTKVEIKHHHHHHEPEA |
| 181 | ACP156 | mdmrvpaqllgllllwlrgarcaptssstkktqlqlehllldlqmilnginnyknpkltrmltfkfympkkatelkhlqcleee |
| | (IL-2 | lkpleevlnlaqsknfhlrprdlisninvivlelkgsettfmceyadetativeflnrwitfcqsiistltsggpPLAQKLKS |
| | Conju- | SpgsEVQLVESGGGLVQPGNSLRLSCAASGFTFSKFGMSWVRQAPGKGLEWVSSI |
| | gate) | SGSGRDTLYAESVKGRFTISRDNAKTTLYLQMNSLRPEDTAVYYCTIGGSLSVSS |
| | | QGTLVTVSSggggsggggsggggsggggsggggsggggssggpPLAQKLKSSpgsEVQLVESGG |
| | | GLVQPGGSLRLSCAASGFTFSSYTLAWVRQAPGKGLEWVAAIDSSSYTYSPDTV |
| | | RGRFTISRDNAKNSLYLQMNSLRAEDTAVYYCARDSNWDALDYWGQGTTVTV |
| | | SSGGGGSGGGGSGGGGSDIQMTQSPSSLSASVGDRVTITCKASQNVGTNVGWY |
| | | QQKPGKAPKALIYSASFRYSGVPSRFSGSGSGTDFTLTISSLQPEDFATYYCQQYY |
| | | TYPYTFGGGTKVEIKHHHHHHEPEA |
| 182 | ACP157 | mdmrvpaqllgllllwlrgarcaptssstkktqlqlehllldlqmilnginnyknpkltrmltfkfympkkatelkhlqcleee |
| | (IL-2 | lkpleevlnlaqsknfhlrprdlisninvivlelkgsettfmceyadetativeflnrwitfcqsiistltsggpPGGPAGIGa |
| | Conju- | lfkssfpPLAQKLKSSpgsEVQLVESGGGLVQPGNSLRLSCAASGFTFSKFGMSWVR |
| | gate) | QAPGKGLEWVSSISGSGRDTLYAESVKGRFTISRDNAKTTLYLQMNSLRPEDTA |
| | | VYYCTIGGSLSVSSQGTLVTVSSggggsggggsggggsggggsggggsggggssggpPGGPAGI |
| | | GalfkssfpPLAQKLKSSpgsEVQLVESGGGLVQPGGSLRLSCAASGFTFSSYTLAWV |
| | | RQAPGKGLEWVAAIDSSSYTYSPDTVRGRFTISRDNAKNSLYLQMNSLRAEDTA |
| | | VYYCARDSNWDALDYWGQGTTVTVSSGGGGSGGGGSGGGGSDIQMTQSPSSL |
| | | SASVGDRVTITCKASQNVGTNVGWYQQKPGKAPKALIYSASFRYSGVPSRFSGS |
| | | GSGTDFTLTISSLQPEDFATYYCQQYYTYPYTFGGGTKVEIKHHHHHHEPEA |
| 183 | | Place Hold |
| 184 | | Place Hold |
| 185 | | Place Hold |
| 186 | | Place Hold |
| 187 | | Place Hold |
| 188 | | Place Hold |
| 189 | | Place Hold |
| 190 | | Place Hold |
| 191 | Blocker 2 | mdmrvpaqllgllllwlrgarcEVQLVESGGGLVQPGGSLRLSCAASGFTFSSYTLAWVRQ |
| | (IL2 | APGKGLEWVAAIDSSSYTYSPDTVRGRFTISRDNAKNSLYLQMNSLRAEDTAVY |
| | blocker) | YCARDSNWDALDYWGQGTTVTVSSggggsggggsggggsDIQMTQSPSSLSASVGDR |
| | | VTITCKASQNVGTNVGWYQQKPGKAPKALIYSASFRYSGVPSRFSGSGSGTDFT |
| | | LTISSLQPEDFATYYCQQYYTYPYTFGGGTKVEIKHHHHHH |
| 192 | Blocker | mdmrvpaqllgllllwlrgarcQSVLTQPPSVSGAPGQRVTISCSGSRSNIGSNTVKWYQQL |
| | 12 (IL-12 | PGTAPKWYYNDQRPSGVPDRFSGSKSGTSASLAITGLQAEDEADYYCQSYDRY |
| | blocker) | THPALLFGTGTKVTVLggggsggggsggggsQVQLVESGGGVVQPGRSLRLSCAASGF |
| | | TFSSYGMHWVRQAPGKGLEWVAFIRYDGSNKYYADSVKGRFTISRDNSKNTLY |
| | | LQMNSLRAEDTAVYYCKTHGSHDNWGQGTMVTVSSHHHHHH |
| 193 | Human_ | cdlpqthslgsrrtlmllaqmrrislfsclkdrhdfgfpqeefgnqfqkaetipvlhemiqqifnlfstkdssaawdetlldkfy |
| | FNA2b | telyqqlndleacviqgvgvtetplmkedsilavrkyfqritlylkekkyspcawevvraeimrsfslstnlqeslrskeHHH |
| | I | HHH** |
| 194 | ACP239- | iwelkkdvyvveldwypdapgemvvltcdtpeedgitwtldqssevlgsgktltiqvkefgdagqytchkggevlshsllll |
| | geneart | hkkedgiwstdilkdqkepknktflrceaknysgrftcwwlttistdltfsvkssrgssdpqgvtcgaatlsaervrgdnkey |
| | | eysvecqedsacpaaeeslpievmvdavhklkyenytssffirdiikpdppknlqlkplknsrqvevsweypdtwstphs |
| | | yfsltfcvqvqgkskrekkdrvftdktsatvicrknasisvraqdryyssswsewasvpcsggggsggggsggggsrvipvsg |
| | | parclsqsrnllkttddmvktareklkhysctaedidheditrdqtstlktclplelhknesclatretssttrgsclppqktslm |
| | | mtlclgsiyedlkmyqtefqainaalqnhnhqqiildkgmlvaidelmqslnhngetlrqkppvgeadpyrvkmklcillh |
| | | afstrvvtinrvmgylssahhhhhh |
| 195 | 3CYT5_sdAb | QVQLQESGGGLVQAGGSLRLSCAASGRTFSSVYDMGWFRQAPGKDREFVARITESARNTRYADSV |
| | | RGRFTISRDNAKNTVYLQMNNLELEDAAVYYCAADPQTVVVGTPDYWGQGTQVTVSSAAAYPYD |
| | | VPDYGSHHHHHH |
| 196 | ACP248 | QSVLTQPPSVSGAPGQRVTISCtGSsSNIGSNTVKWYQQLPGTAPKLLIYgN |
| | | DQRPSGVPDRFSGSKSGTSASLAITGLQAEDEADYYCQSYDRYTHPAyvF |
| | | GTGTKVTVLggggsggggsggggsQVQLVESGGGVVQPGRSLRLSCAASGFTFS |
| | | SYGMHWVRQAPGKGLEWVAFIRYDGSNKYYADSVKGRFTISRDNSKNT |
| | | LYLQMNSLRAEDTAVYYCKTHGSHDNWGQGTMVTVSSHHHHHHR |
| 197 | ACP249 | QSVLTQPPSVSGAPGQRVTISCtGSsSNIGSNTVKWYQQLPGTAPKLLIYYNDQRP |
| | | SGVPDRFSGSKSGTSASLAITGLQAEDEADYYCQSYDRYTHPAyvFGTGTKVTVL |
| | | ggggsggggsggggsQVQLVESGGGVVQPGRSLRLSCAASGFTFSSYGMHWVRQAPG |
| | | KGLEWVAFIRYDGSNKYYADSVKGRFTISRDNSKNTLYLQMNSLRAEDTAVYY |
| | | CKTHGSHDNWGQGTMVTVSSHHHHHH |
| 198 | ACP250 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYaMHWVRQAP |
| | | GKGLEWVAvIsYDGSNKYYADSVKGRFTISRDNSKNTLYLQMNSLRAEDTAVY |
| | | YCarHGSHDNWGQGTMVTVSSHHHHHH |
| 199 | ACP251 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYeGSNKYYADSVKGRFTISRDNSKNTLYLQMNSLRAEDTAVY |
| | | YCKTHGSHDNWGQGTMVTVSSHHHHHH |
| 200 | ACP252 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYAeSVKGRFTISRDNSKNTLYLQMNSLRAEDTAVY |
| | | YCKTHGSHDNWGQGTMVTVSSHHHHHH |
| 201 | ACP253 | QSVLTQPPSVSGAPGQRVTISCSGSRSNIGSqTVKWYQQLPGTAPKLLIYYNDQR |
| | | PSGVPDRFSGSKSGTSASLAITGLQAEDEADYYCQSYeRYTHPALLFGTGTKVTV |
| | | LggggsggggsggggsQVQLVESGGGVVQPGRSLRLSCAASGFTFSSYGMHWVRQAP |
| | | GKGLEWVAFIRYDGSNKYYADSVKGRFTISRDNSKNTLYLQMNSLRAEDTAVY |
| | | YCKTHGSHDNWGQGTMVTVSSHHHHHH |
| 202 | ACP254 | QSVLTQPPSVSGAPGQRVTISCSGSRSNIGSqTVKWYQQLPGTAPKLLIYYNDQR |
| | | PSGVPDRFSGSKSGTSASLAITGLQAEDEADYYCQSYsRYTHPALLFGTGTKVTV |
| | | LggggsggggsggggsQVQLVESGGGVVQPGRSLRLSCAASGFTFSSYGMHWVRQAP |
| | | GKGLEWVAFIRYDGSNKYYADSVKGRFTISRDNSKNTLYLQMNSLRAEDTAVY |
| | | YCKTHGSHDNWGQGTMVTVSSHHHHHH |
| 203 | ACP255 | QSVLTQPPSVSGAPGQRVTISCSGSeSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH |
| 204 | ACP256 | QSVLTQPPSVSGAPGQRVTISCSGSsSNIGSNTVKWYQQLPGTAPKLLIYYNDQRP |
| | | SGVPDRFSGSKSGTSASLAITGLQAEDEADYYCQSYDRYTHPALLFGTGTKVTV |
| | | LggggsggggsggggsQVQLVESGGGVVQPGRSLRLSCAASGFTFSSYGMHWVRQAP |
| | | GKGLEWVAFIRYDGSNKYYADSVKGRFTISRDNSKNTLYLQMNSLRAEDTAVY |
| | | YCKTHGSHDNWGQGTMVTVSSHHHHHH |
| 205 | ACP257 | QSVLTQPPSVSGAPGQRVTISCSGSRSNIGdNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH |
| 206 | ACP258 | QSVLTQPPSVSGAPGQRVTISCSGSRSNIGeNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH |
| 207 | ACP259 | QSVLTQPPSVSGAPGQRVTISCSGSRSNIGSdTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH |
| 208 | ACP260 | QSVLTQPPSVSGAPGQRVTISCSGSRSNIGSeTVKWYQQLPGTAPKLLIYYNDQRP |
| | | SGVPDRFSGSKSGTSASLAITGLQAEDEADYYCQSYDRYTHPALLFGTGTKVTV |
| | | LggggsggggsggggsQVQLVESGGGVVQPGRSLRLSCAASGFTFSSYGMHWVRQAP |
| | | GKGLEWVAFIRYDGSNKYYADSVKGRFTISRDNSKNTLYLQMNSLRAEDTAVY |
| | | YCKTHGSHDNWGQGTMVTVSSHHHHHH |
| 209 | ACP261 | QSVLTQPPSVSGAPGQRVTISCSGSRSNIGSNdVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH |
| 210 | ACP262 | QSVLTQPPSVSGAPGQRVTISCSGSRSNIGSNTVdWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH |
| 211 | ACP263 | QSVLTQPPSVSGAPGQRVTISCSGSRSNIGSNTVeWYQQLPGTAPKLLIYYNDQRP |
| | | SGVPDRFSGSKSGTSASLAITGLQAEDEADYYCQSYDRYTHPALLFGTGTKVTV |
| | | LggggsggggsggggsQVQLVESGGGVVQPGRSLRLSCAASGFTFSSYGMHWVRQAP |
| | | GKGLEWVAFIRYDGSNKYYADSVKGRFTISRDNSKNTLYLQMNSLRAEDTAVY |
| | | YCKTHGSHDNWGQGTMVTVSSHHHHHH |
| 212 | ACP264 | QSVLTQPPSVSGAPGQRVTISCSGSRSNIGSNTVKWYQQLPGTAPKLLIYYNDQd |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH |
| 213 | ACP265 | QSVLTQPPSVSGAPGQRVTISCSGSRSNIGSNTVKWYQQLPGTAPKLLIYYNDQe |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH |
| 214 | ACP266 | QSVLTQPPSVSGAPGQRVTISCSGSRSNIGSNTVKWYQQLPGTAPKLLIYYNDQR |
| | | PdGVPDRFSGSKSGTSASLAITGLQAEDEADYYCQSYDRYTHPALLFGTGTKVTV |
| | | LggggsggggsggggsQVQLVESGGGVVQPGRSLRLSCAASGFTFSSYGMHWVRQAP |
| | | GKGLEWVAFIRYDGSNKYYADSVKGRFTISRDNSKNTLYLQMNSLRAEDTAVY |
| | | YCKTHGSHDNWGQGTMVTVSSHHHHHH |
| 215 | ACP267 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDeYTHPALLFGTGTKVTV |
| | | LggggsggggsggggsQVQLVESGGGVVQPGRSLRLSCAASGFTFSSYGMHWVRQAP |
| | | GKGLEWVAFIRYDGSNKYYADSVKGRFTISRDNSKNTLYLQMNSLRAEDTAVY |
| | | YCKTHGSHDNWGQGTMVTVSSHHHHHH |
| 216 | ACP268 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTdPALLFGTGTKVTV |
| | | LggggsggggsggggsQVQLVESGGGVVQPGRSLRLSCAASGFTFSSYGMHWVRQAP |
| | | GKGLEWVAFIRYDGSNKYYADSVKGRFTISRDNSKNTLYLQMNSLRAEDTAVY |
| | | YCKTHGSHDNWGQGTMVTVSSHHHHHH |
| 217 | ACP269 | QSVLTQPPSVSGAPGQRVTISCSGSeSNIGSNTVKWYQQLPGTAPKLLIYYNDQeP |
| | | SGVPDRFSGSKSGTSASLAITGLQAEDEADYYCQSYDeYTHPALLFGTGTKVTVL |
| | | ggggsggggsggggsQVQLVESGGGVVQPGRSLRLSCAASGFTFSSYGMHWVRQAPG |
| | | KGLEWVAFIRYDGSNKYYADSVKGRFTISRDNSKNTLYLQMNSLRAEDTAVYY |
| | | CKTHGSHDNWGQGTMVTVSSHHHHHH |
| 218 | ACP270 | QSVLTQPPSVSGAPGQRVTISCSGSeSNIGSNdVKWYQQLPGTAPKLLIYYNDQRP |
| | | SGVPDRFSGSKSGTSASLAITGLQAEDEADYYCQSYDRYTHPALLFGTGTKVTV |
| | | LggggsggggsggggsQVQLVESGGGVVQPGRSLRLSCAASGFTFSSYGMHWVRQAP |
| | | GKGLEWVAFIRYDGSNKYYADSVKGRFTISRDNSKNTLYLQMNSLRAEDTAVY |
| | | YCKTHGSHDNWGQGTMVTVSSHHHHHH |
| 219 | ACP271 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFeSYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH |
| 220 | ACP272 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSeYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH |
| 221 | ACP273 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSdYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH |
| 222 | ACP274 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIeYDGSNKYYADSVKGRFTISRDNSKNTLYLQMNSLRAEDTAVY |
| | | YCKTHGSHDNWGQGTMVTVSSHHHHHH |
| 223 | ACP275 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIdYDGSNKYYADSVKGRFTISRDNSKNTLYLQMNSLRAEDTAV |
| | | YYCKTHGSHDNWGQGTMVTVSSHHHHHH |
| 224 | ACP276 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNdYYADSVKGRFTISRDNSKNTLYLQMNSLRAEDTAVY |
| | | YCKTHGSHDNWGQGTMVTVSSHHHHHH |
| 225 | ACP277 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNeYYADSVKGRFTISRDNSKNTLYLQMNSLRAEDTAVY |
| | | YCKTHGSHDNWGQGTMVTVSSHEIHHHH |
| 226 | ACP278 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVeGRFTISRDNSKNTLYLQMNSLRAEDTAVY |
| | | YCKTHGSHDNWGQGTMVTVSSHHHHHH |
| 227 | ACP279 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVKGRFTISRDNSKNTLYLQMNSLRAEDTAV |
| | | YYCKTHGSeDNWGQGTMVTVSSHHHHHH |
| 228 | ACP280 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIeYDGSNKYYADSVeGRFTISRDNSKNTLYLQMNSLRAEDTAVY |
| | | YCKTHGSHDNWGQGTMVTVSSHHHHHH |
| 229 | ACP281 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIeYDGSNKYYADSVeGRFTISRDNSKNTLYLQMNSLRAEDTAVY |
| | | YCKTHGSeDNWGQGTMVTVSSHHHHHH |
| 230 | ACP282 | QSVLTQPPSVSGAPGQRVTISCSGSRSNIGSNTVKWYQQLPGTAPKLLIYYNDQR |
| | | PSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHEIREIHH |
| 231 | ACP283 | iwelkkdvyvveldwypdapgemvvltcdtpeedgitwtldqssevlgsgtltiqvkefgdagqytchkggevlshslll |
| | | lhkkedgiwstdilkdqkepknktflrceaknysgrftcwwlttistdltfsvkssrgssdpqgvtcgaatlsaervrgdnkey |
| | | eysvecqedsacpaaeeslpievmvdavhklkyenytssffirdiikpdppknlqlkplknsrqvevsweypdtwstphs |
| | | yfsltfcvqvqgkskrekkdrvftdktsatvicrknasisvraqdryyssswsewasvpcs |
| 232 | 3TOW6 | QVQLQESGGGLVQTGGSLRLSCTTSGTIFSGYTMGWYRQAPGEQRELVA |
| | 9sdAb | VISGGGDTNYADSVKGRFTISRDNTKDTMYLQMNSLKPEDTAVYYCYSR |
| | | EVTPPWKLYWGQGTQVTVSSAAAYPYDVPDYGSHHHHHH |
| 233 | 3TOW85 | QVQLQESGGGLVQEGGSLRLSCAASERIFSTDVMGWYRQAAEKQRELVAVVSA |
| | sdAb | RGTTNYLDAVKGRFTISRDNARNTLTLQMNDLKPEDTASYYCYVRETTSPWRIY |
| | | WGQGTQVTVSSAAAYPYDVPDYGSHHHHHH |
| 234 | 2TOW91 | QVQLQESGGGLVQAGGSLRLSCAASGSIFSANAMGWYRQAPGKQRELVAVISS |
| | sdAb | GGSTNYADSVKGRFTISRDNAKNTVYLQMNSLKPEDTAVYYCMYSGSYYYTPN |
| | | DYWGQGTQVTVSSAAAYPYDVPDYGSHHHHHH |
| 235 | ACP301 | evqlvesggglvqpggslrlscaasgftfssytlawvrqapgkglewvaaidsssvtvspdtvrgrftisrdnakns |
| | | lylqmnslraedtavyycardsnwdaldywgqgttvtvssggggsggggsggggsdiqmtqspsslsasvgdr |
| | | vtitckasqnvgtnvgwyqqkpgkapkaliysasfrysgvpsrfsgsgsgtdftltisslqpedfatyycqqyvtv |
| | | pytfgggtkveikhhhhhh |
| 236 | Hu2TOW91_ | evqllesggglvqpggslrlscaasGSIFSANAMGwYrqapgkQReLvAVISSGGSTNYAD |
| | A | SVKGrftisrdnskntVylqmnslraedtavyycMYSGSYYYTPNDYwgqgtlvtvssAAAY |
| | | PYDVPDYGSHHHHHH** |
| 237 | Hu2TOW91_ | evqllesggglvqpggslrlscaasGSIFSANAMGwYrqapgkgleLvAVISSGGSTNYADSVKGrft |
| | B | isrdnskntVylqmnslraedtavyycMYSGSYYYTPNDYwgqgtlvtvssAAAYPYDVPDYGSH |
| | | HHHHH** |
| 238 | Hu2TOW91_ | evqllesggglvqpggslrlscaasGSIFSANAMGwvrqapgkglewvsVISSGGSTNYADSVKGrftis |
| | C | rdnskntlylqmnslraedtavyycMYSGSYYYTPNDYwgqgtlvtvssAAAYPYDVPDYGSHHH |
| | | HHH** |
| 239 | Hu2TOW91_ | QvqllesggglyqpggslrlscaasGSIFSANAMGwYrqapgkQReLvAVISSGGSTNYADSVKG |
| | D | rftisrdnskntVylqmnslraedtavyycMYSGSYYYTPNDYwgqgtlVtVssAAAYPYDVPDYGS |
| | | HHHHHH** |
| 240 | HE_LM_ | evqLlesggglVqpggslrlscaasgSIfsANamGwYrqapgkgReLvAVissggstNyadsvkgrftisrdnsknt |
| | 2TOW91 | VylqmnslraedtavyycMYSGSYYYTPNDYWgqgtlvtvssAAAYPYDVPDYGSHHHHHH |
| | | ** |
| 241 | HE_L_ | QvqllesggglvqAggslrlscaasgSIfsANamGwYrqapgkQReLvAVissggstNyadsvkgrftisrdnsk |
| | 2TOW91 | ntVylqmnslraedtavyycMYSGSYYYTPNDYwgqgtlvtvssAAAYPYDVPDYGSHHHHH |
| | | H** |
| 242 | Hu3TOW85_ | evqllesggglvqpggslrlscaasERIFSTDVMGwYrqapgkQReLvAVVSARGTTNYLDAVKG |
| | A | rftisrdnskntlylqmnslraedtavyycYVRETTSPWRIYwgqgtlvtvssAAAYPYDVPDYGSHH |
| | | HHHH** |
| 243 | Hu3TOW85_ | evqllesggglvqpggslrlscaasERIFSTDVMGwYrqapgkgleLvAVVSARGTTNYLDAVKGrf |
| | B | tisrdnskntlylqmnslraedtavyycYVRETTSPWRIYwgqgtlvtvssAAAYPYDVPDYGSHHH |
| | | HHH** |
| 244 | Hu3TOW85_ | evqllesggglvqpggslrlscaasERIFSTDVMGwvrqapgkglewvsVVSARGTTNYLDAVKGrft |
| | C | isrdnskntlylqmnslraedtavyycYVRETTSPWRIYwgqgtlvtvssAAAYPYDVPDYGSHHH |
| | | HHH** |
| 245 | Hu3TOW85_ | QvqllesggglvqpggslrlscaasERIFSTDVMGwYrqapgkQReLvAVVSARGTTNYLDAVK |
| | D | GrftisrdnskntlylqmnslraedtavyycYVRETTSPWRIYwgqgtlvtvssAAAYPYDVPDYGSH |
| | | HHHHH** |
| 246 | HE_LM_ | evqllesggglvqpggslrlscaasERIfsTDVmGwYrqapgkgReLvAVVsARgTtNyLdsvkgrftisrdn |
| | 3TOW85 | skntlylqmnslraedtavyycYVRETTSPWRIywgqgtlvtvssAAAYPYDVPDYGSHHHHHH* |
| | | * |
| 247 | HE_L_ | QvqllesggglvqEggslrlscaasERIfsTDVmGwYrqaAgkQReLvAVVsARgTtNyLdAvkgrftis |
| | 3TOW85 | rdnskntlylqmnslraedtaSyycYVRETTSPWRIywgqgtlvtvssAAAYPYDVPDYGSHHHHH |
| | | H** |
| 248 | HE_LM_ | evqllesggglvqpggslrlscaasERIfsTDVmGwYrqapgkgleLvAVVsARgTtNyLdsvkgrftisrdns |
| | R45_L3TOW85 | kntlylqmnslraedtavyycYVRETTSPWRIywgqgtlvtvssAAAYPYDVPDYGSHHHHHH** |
| 249 | Hu3TOW69_ | evqllesggglvqpggslrlscaTsGTIFSGYTMGwYrqapgkQReLvAVISGGGDTNYADSVKG |
| | A | rftisrdnskDtMylqmnslraedtavyycYSREVTPPWKLYwgqgtlvtvssAAAYPYDVPDYGSH |
| | | HHHHH** |
| 250 | Hu3TOW69_ | evqllesggglvqpggslrlscaTsGTIFSGYTMGwYrqapgkgleLvAVISGGGDTNYADSVKGrf |
| | B | tisrdnskDtMylqmnslraedtavyycYSREVTPPWKLYwgqgtlvtvssAAAYPYDVPDYGSHH |
| | | HHHH** |
| 251 | Hu3TOW69_ | evqllesggglvqpggslrlscaasGTIFSGYTMGwvrqapgkglewvsVISGGGDTNYADSVKGrfti |
| | C | srdnskntlylqmnslraedtavyycYSREVTPPWKLYwgqgtlvtvssAAAYPYDVPDYGSHHH |
| | | HHH** |
| 252 | Hu3TOW69_ | QvqllesggglvqpggslrlscaTsGTIFSGYTMGwYrqapgkQReLvAVISGGGDTNYADSVK |
| | D | GrftisrdnskDtMylqmnslraedtavyycYSREVTPPWKLYwgqgtlvtvssAAAYPYDVPDYGS |
| | | HHHHHH** |
| 253 | Hu3TOW69_ | evqllesggglvqpggslrlscaTsGTIFSGYTMGwYrqapgkQReLvAVISGGGDTNYADSVKG |
| | E | rftisrdnskntMylqmnslraedtavyycYSREVTPPWKLYwgqgtlvtvssAAAYPYDVPDYGSH |
| | | HHHHH** |
| 254 | HE_LM_ | evqllesggglvqpggslrlscaTsgTIfsGyTmGwYrqapgkgReLvAVisGggDtNyadsvkgrftisrdnsk |
| | 3TOW69 | ntMylqmnslraedtavyycYSREVTPPWKLywgqgtlvtvssAAAYPYDVPDYGSHHHHHH* |
| | | * |
| 255 | HE_L_ | QvqllesggglvqTggslrlscaTsgTIfsGyTmGwYrqapgkQReLvAVisGggDtNyadsvkgrftisrdn |
| | 3TOW69 | skDtMylqmnslraedtavyycYSREVTPPWKLywgqgtlvtvssAAAYPYDVPDYGSHHHHH |
| | | H** |
| 256 | HE_LM_ | evqllesggglvqpggslrlscaTsgTIfsGyTmGwYrqapgkgleLvAVisGggDtNyadsvkgrftisrdnsk |
| | R45L_ | ntMylqmnslraedtavyycYSREVTPPWKLywgqgtlvtvssAAAYPYDVPDYGSHHHHHH* |
| | 3TOW69 | * |
| 257 | ACP363 | EVQLVESGGGLVQPGGSLRLSCAASGFTFSSYTLAWVRQAPGKGLEWVA |
| | | AIDSSSYTYSPDTVRGRFTISRDNAKNSLYLQMNSLRAEDTAVYYCARDS |
| | | NWDALDYWGQGTTVTVSSggggsggggsggggsDIQMTQSPSSLSASVGDRVT |
| | | ITCKAREKLWSAVAWYQQKPGKAPKALIYSASFRYSGVPSRFSGSGSGTD |
| | | FTLTISSLQPEDFATYYCQQYYTYPYTFGGGTKVEIKHHHHHH |
| 258 | ACP364 | EVQLVESGGGLVQPGGSLRLSCAASGFTFSSYTLAWVRQAPGKGLEWVAAIDSS |
| | | SYTYSPDTVRGRFTISRDNAKNSLYLQMNSLRAEDTAVYYCARDSNWDALDYW |
| | | GQGTTVTVSSggggsggggsggggsDIQMTQSPSSLSASVGDRVTITCKAREKLWSAV |
| | | AWYQQKPGKAPKsLIYSASFRYSGVPSRFSGSGSGTDFTLTISSLQPEDFATYYCQ |
| | | QYYTYPYTFGGGTKVEIKHHHHHH |
| 259 | ACP367 | EVQLVESGGGLVQPGGSLRLSCAASGFTFSSYTLAWVRQAPGKGLEWVA |
| | | AIDSSSYTYSPDTVRGRFTISRDNAKNSLYLQMNSLRAEDTAVYYCARDS |
| | | NWDALDYWGQGTTVTVSSggggsggggsggggsDIQMTQSPSSLSASVGDRVT |
| | | ITCKVTEKVWGNVAWYQQKPGKAPISLIYSPSLRKSGVPSRFSGSGSGTD |
| | | FTLTISSLQPEDFATYYCQQYYTYPYTFGGGTKVEIKHHHHHH |
| 260 | ACP369 | EVQLVESGGGLVQPGGSLRLSCAASGFTFSSYTLAWVRQAPGKGLEWVA |
| | | AIDSSSYTYSPDTVRGRFTISRDNAKNSLYLQMNSLRAEDTAVYYCARDS |
| | | NWDALDYWGQGTTVTVSSggggsggggsggggsDIQMTQSPSSLSASVGDRVT |
| | | ITCKSSEKLWANVAWYQQKPGKAPKALIYSASFRYSGVPSRFSGSGSGTD |
| | | FTLTISSLQPEDFATYYCQQYYTYPYTFGGGTKVEIKHHHHHH |
| 261 | ACP370 | EVQLVESGGGLVQPGGSLRLSCAASGFTFSSYTLAWVRQAPGKGLEWVAAIDSS |
| | | SYTYSPDTVRGRFTISRDNAKNSLYLQMNSLRAEDTAVYYCARDSNWDALDYW |
| | | GQGTTVTVSSggggsggggsggggsDIQMTQSPSSLSASVGDRVTITCKSSEKLWANVA |
| | | WYQQKPGKAPKsLIYSASFRYSGVPSRFSGSGSGTDFTLTISSLQPEDFATYYCQQ |
| | | YYTYPYTFGGGTKVEIKHHHHHH |
| 262 | ACP380 | DIQMTQSPSSLSASVGDRVTITCKASQNVGTNVGWYQQKPGKAPKALIY |
| | | SASFRYSGVPSRFSGSGSGTDFTLTISSLQPEDFATYYCQQYYTYPYTFGG |
| | | GTKVEIKrtvaapsvfifppsdeqlksgtasvvellnnfypreakvqwkvdnalqsgnsqesvteqdskdst |
| | | yslsstitlskadyekhkvyacevthqglsspvtksfnrgec |
| 263 | ACP381 | DIQMTQSPSSLSASVGDRVTITCKAREKLWSAVAWYQQKPGKAPKsLIYSASFR |
| | | YSGVPSRFSGSGSGTDFTLTISSLQPEDFATYYCQQYYTYPYTFGGGTKVEIKrtvaa |
| | | psvfifppsdeqlksgtasvvcllnnfypreakvqwkvdnalqsgnsqesvteqdskdstyslsstltlskadyekhkvyac |
| | | evthqglsspvtksfnrgec |
| 264 | ACP382 | DIQMTQSPSSLSASVGDRVTITCKVTEKVWGNVAWYQQKPGKAPISLIYSPSLRK |
| | | SGVPSRFSGSGSGTDFTLTISSLQPEDFATYYCQQYYTYPYTFGGGTKVEIKrtvaap |
| | | svfifppsdeqlksgtasvvcllnnfypreakvqwkvdnalqsgnsqesvteqdskdstyslssthlskadyekhkvyace |
| | | vthqglsspvtksfnrgec |
| 265 | ACP435 | DIQMTQSPSSLSASVGDRVTITCKAREKLWSAVAWYQQKPGKAPKsLIYS |
| | | ASFRYSGVPSRFSGSGSGTDFTLTISSLQPEDFATYYCQQYYTYPYTFGGG |
| | | TKVEIKrtvaapsvfifppsdeqlksgtasvvcllnnfypreakvqwkvdnalqsgnsqesvteqdskdsty |
| | | slsstltlskadyekhkvyacevthqglsspvtksfnrgecggggsggggsggggsggggsggggsggggsev |
| | | qllesggglvqpggslrlscaasgsifsanamgwyrqapgkqrelvavissggstnyadsvkgrftisrdnskntv |
| | | ylqmnslraedtavyycmysgsyyytpndywgqgtlvtvss** |
| 266 | ACP436 | DIQMTQSPSSLSASVGDRVTITCKAREKLWSAVAWYQQKPGKAPKsLIYSASFR |
| | | YSGVPSRFSGSGSGTDFTLTISSLQPEDFATYYCQQYYTYPYTFGGGTKVEIKrtvaa |
| | | psvfifppsdeqlksgtasvvcllnnfypreakvqwkvdnalqsgnsqesvteqdskdstyslsstltlskadyekhkvyac |
| | | evthqglsspvtksfnrgecggggsggggsggggsggggsggggsggggsevqllesggglvqpggslrlscaasgsifsa |
| | | namgwyrqapgkglelvavissggstnyadsvkgrftisrdnskntvylqmnslraedtavyycmysgsyyytpndyw |
| | | gqgtlvtvss** |
| 267 | ACP437 | DIQMTQSPSSLSASVGDRVTITCKVTEKVWGNVAWYQQKPGKAPISLIYSPSLRK |
| | | SGVPSRFSGSGSGTDFTLTISSLQPEDFATYYCQQYYTYPYTFGGGTKVEIKrtvaap |
| | | svfifppsdeqlksgtasvvcllnnfypreakvqwkvdnalqsgnsqesvteqdskdstyslssthlskadyekhkvyace |
| | | vthqglsspvtksfnrgecggggsggggsggggsggggsggggsggggsevqllesggglvqpggslrlscaasgsifsan |
| | | amgwyrqapgkqrelvavissggstnyadsvkgrftisrdnskntvylqmnslraedtavyycmysgsyyytpndywg |
| | | qgtlvtvss** |
| 268 | ACP438 | DIQMTQSPSSLSASVGDRVTITCKVTEKVWGNVAWYQQKPGKAPISLIYSPSLRK |
| | | SGVPSRFSGSGSGTDFTLTISSLQPEDFATYYCQQYYTYPYTFGGGTKVEIKrtvaap |
| | | svfifppsdeqlksgtasvvcllnnfypreakvqwkvdnalqsgnsqesvteqdskdstyslsstltskadyekhkvyace |
| | | vthqglsspvtksfnrgecggggsggggsggggsggggsggggsggggsevqllesggglvqpggslrlscaasgsifsan |
| | | amgwyrqapgkglelvavissggstnyadsvkgrftisrdnskntvylqmnslraedtavyycmysgsyyytpndywg |
| | | qgtlvtvss** |
| 269 | ACP448 | DIQMTQSPSSLSASVGDRVTITCKSSEKLWANVAWYQQKPGKAPKsLIYS |
| | | ASFRYSGVPSRFSGSGSGTDFTLTISSLQPEDFATYYCQQYYTYPYTFGGG |
| | | TKVEIKrtvaapsvfifppsdeqlksgtasvvcllnnfypreakvqwkvdnalqsgnsqesvteqdskdsty |
| | | slsstltlskadyekhkvyaceythqglsspytksfnrgec** |
| 270 | ACP449 | DIQMTQSPSSLSASVGDRVTITCKSSEKLWANVAWYQQKPGKAPKLLIYSASFR |
| | | YSGVPSRFSGSGSGTDFTLTISSLQPEDFATYYCQQYYTYPYTFGGGTKVEIKrtvaa |
| | | psvfifppsdeqlksgtasvvcllnnfypreakvqwkvdnalqsgnsqesvteqdskdstyslsstltlskadyekhkvyac |
| | | evthqglsspvtksfnrgec** |
| 271 | ACP450 | DIQMTQSPSSLSASVGDRVTITCKAREKLWSAVAWYQQKPGKAPKLLIYSASFR |
| | | YSGVPSRFSGSGSGTDFTLTISSLQPEDFATYYCQQYYTYPYTFGGGTKVEIKrtvaa |
| | | psvfifppsdeqlksgtasvvcllnnfypreakvqwkvdnalqsgnsqesvteqdskdstyslsstltlskadyekhkvyac |
| | | evthqglsspvtksfnrgec** |
| 272 | ACP439 | aptssstkktqlqlehllldlqmilnginnyknpkltrmltfkfympkkatelkhlgcleeelkpleevlnlaqsknf |
| | | hlrprdlisninvivlelkgsettfmceyadetativeflnrwitfcqsiistltsggpGPAGLYAQpgsEVQ |
| | | LVESGGGLVQPGNSLRLSCAASGFTFSKFGMSWVRQAPGKGLEWVSSIS |
| | | GSGRDTLYAESVKGRFTISRDNAKTTLYLQMNSLRPEDTAVYYCTIGGSL |
| | | SVSSQGTLVTVSSggggsggggsggggsggggsggggsggggssggpGPAGLYAQpgsEV |
| | | QLVESGGGLVQPGGSLRLSCAASGFTFSSYTLAWVRQAPGKGLEWVAAI |
| | | DSSSYTYSPDTVRGRFTISRDNAKNSLYLQMNSLRAEDTAVYYCARDSN |
| | | WDALDYWGQGTTVTVSSGGGGSGGGGSGGGGSDIQMTQSPSSLSASVG |
| | | DRVTITCKSSEKLWANVAWYQQKPGKAPKALIYSASFRYSGVPSRFSGSG |
| | | SGTDFTLTISSLQPEDFATYYCQQYYTYPYTFGGGTKVEIK |
| 273 | ACP440 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGQGTTVTVSSGGGGSGGGGSGGG |
| | | GSDIQMTQSPSSLSASVGDRVTITCKSSEKLWANVAWYQQKPGKAPKsLIYSASF |
| | | RYSGVPSRFSGSGSGTDFTLTISSLQPEDFATYYCQQYYTYPYTFGGGTKVEIK |
| 274 | ACP441 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGQGTTVTVSSGGGGSGGGGSGGG |
| | | GSDIQMTQSPSSLSASVGDRVTITCKSSEKLWANVAWYQQKPGKAPKLLIYSAS |
| | | FRYSGVPSRFSGSGSGTDFTLTISSLQPEDFATYYCQQYYTYPYTFGGGTKVEIK |
| 275 | ACP442 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKcLEWVAAIDSSSYTYSPDTVRGRFTISRDNAKNSLYL |
| | | QMNSLRAEDTAVYYCARDSNWDALDYWGQGTTVTVSSGGGGSGGGGSGGGG |
| | | SDIQMTQSPSSLSASVGDRVTITCKSSEKLWANVAWYQQKPGKAPKsLIYSASFR |
| | | YSGVPSRFSGSGSGTDFTLTISSLQPEDFATYYCQQYYTYPYTFGcGTKVEIK |
| 276 | ACP443 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKcLEWVAAIDSSSYTYSPDTVRGRFTISRDNAKNSLYL |
| | | QMNSLRAEDTAVYYCARDSNWDALDYWGQGTTVTVSSGGGGSGGGGSGGGG |
| | | SDIQMTQSPSSLSASVGDRVTITCKSSEKLWANVAWYQQKPGKAPKLLIYSASFR |
| | | YSGVPSRFSGSGSGTDFTLTISSLQPEDFATYYCQQYYTYPYTFGcGTKVEIK |
| 277 | ACP444 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGcGTTVTVSSGGGGSGGGGSGGGG |
| | | SDIQMTQSPSSLSASVGDRVTITCKSSEKLWANVAWYQQKPGKcPKALIYSASFR |
| | | YSGVPSRFSGSGSGTDFTLTISSLQPEDFATYYCQQYYTYPYTFGGGTKVEIK |
| 278 | ACP445 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGQGTTVTVSSsggpGPAGLYAQpgsD |
| | | IQMTQSPSSLSASVGDRVTITCKAREKLWSAVAWYQQKPGKAPKWYSASFRY |
| | | SGVPSRFSGSGSGTDFTLTISSLQPEDFATYYCQQYYTYPYTFGGGTKVEIK |
| 279 | ACP446 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGQGTTVTVSSGGGGSGGGGSGGG |
| | | GSDIQMTQSPSSLSASVGDRVTITCKAREKLWSAVAWYQQKPGKAPKWYSAS |
| | | FRYSGVPSRFSGSGSGTDFTLTISSLQPEDFATYYCQQYYTYPYTFGGGTKVEIK |
| 280 | ACP447 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKcLEWVAAIDSSSYTYSPDTVRGRFTISRDNAKNSLYL |
| | | QMNSLRAEDTAVYYCARDSNWDALDYWGQGTTVTVSSGGGGSGGGGSGGGG |
| | | SDIQMTQSPSSLSASVGDRVTITCKAREKLWSAVAWYQQKPGKAPKLLIYSASF |
| | | RYSGVPSRFSGSGSGTDFTLTISSLQPEDFATYYCQQYYTYPYTFGcGTKVEIK |
| 281 | ACP451 | aptssstkktqlqlehllldlqmilnginnyknpkltrmltfldympkkatelkhlqcleeelkpleevlnlaqsknf |
| | | hlrprdlisninvivlelkgsettfmceyadetativeflnrwitfcqsiistltsggpALFKSSFPpgsEVQL |
| | | VESGGGLVQPGNSLRLSCAASGFTFSKFGMSWVRQAPGKGLEWVSSISG |
| | | SGRDTLYAESVKGRFTISRDNAKTTLYLQMNSLRPEDTAVYYCTIGGSLS |
| | | VSSQGTLVTVSSggggsggggsggggsggggsggggsggggssggpALFKSSFPpgsEVQL |
| | | VESGGGLVQPGGSLRLSCAASGFTFSSYTLAWVRQAPGKGLEWVAAIDS |
| | | SSYTYSPDTVRGRFTISRDNAKNSLYLQMNSLRAEDTAVYYCARDSNWD |
| | | ALDYWGQGTTVTVSSGGGGSGGGGSGGGGSDIQMTQSPSSLSASVGDRV |
| | | TITCKAREKLWSAVAWYQQKPGKAPKsLIYSASFRYSGVPSRFSGSGSGT |
| | | DFTLTISSLQPEDFATYYCQQYYTYPYTFGGGTKVEIK** |
| 282 | ACP452 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpALFKSSFPpgsEVQLVESGGGLVQ |
| | | PGNSLRLSCAASGFTFSKFGMSWVRQAPGKGLEWVSSISGSGRDTLYAESVKGR |
| | | FTISRDNAKTTLYLQMNSLRPEDTAVYYCTIGGSLSVSSQGTLVTVSSggggsggggs |
| | | ggggsggggsggggsggggssggpALFKSSFPpgsEVQLVESGGGLVQPGGSLRLSCAASGF |
| | | TFSSYTLAWVRQAPGKGLEWVAAIDSSSYTYSPDTVRGRFTISRDNAKNSLYLQ |
| | | MNSLRAEDTAVYYCARDSNWDALDYWGQGTTVTVSSGGGGSGGGGSGGGGS |
| | | DIQMTQSPSSLSASVGDRVTITCKVTEKVWGNVAWYQQKPGKAPISLIYSPSLRK |
| | | SGVPSRFSGSGSGTDFTLTISSLQPEDFATYYCQQYYTYPYTFGGGTKVEIK** |
| 283 | ACP453 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpALFKSSFPpgsEVQLVESGGGLVQ |
| | | PGNSLRLSCAASGFTFSKFGMSWVRQAPGKGLEWVSSISGSGRDTLYAESVKGR |
| | | FTISRDNAKTTLYLQMNSLRPEDTAVYYCTIGGSLSVSSQGTLVTVSSggggsggggs |
| | | ggggsggggsggggsggggssggpALFKSSFPpgsEVQLVESGGGLVQPGGSLRLSCAASGF |
| | | TFSSYTLAWVRQAPGKcLEWVAAIDSSSYTYSPDTVRGRFTISRDNAKNSLYLQ |
| | | MNSLRAEDTAVYYCARDSNWDALDYWGQGTTVTVSSGGGGSGGGGSGGGGS |
| | | DIQMTQSPSSLSASVGDRVTITCKAREKLWSAVAWYQQKPGKAPKsLIYSASFR |
| | | YSGVPSRFSGSGSGTDFTLTISSLQPEDFATYYCQQYYTYPYTFGcGTKVEIK** |
| 284 | ACP454 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpALFKSSFPpgsEVQLVESGGGLVQ |
| | | PGNSLRLSCAASGFTFSKFGMSWVRQAPGKGLEWVSSISGSGRDTLYAESVKGR |
| | | FTISRDNAKTTLYLQMNSLRPEDTAVYYCTIGGSLSVSSQGTLVTVSSggggsggggs |
| | | ggggsggggsggggsggggssggpALFKSSFPpgsEVQLVESGGGLVQPGGSLRLSCAASGF |
| | | TFSSYTLAWVRQAPGKGLEWVAAIDSSSYTYSPDTVRGRFTISRDNAKNSLYLQ |
| | | MNSLRAEDTAVYYCARDSNWDALDYWGcGTTVTVSSGGGGSGGGGSGGGGSD |
| | | IQMTQSPSSLSASVGDRVTITCKAREKLWSAVAWYQQKPGKcPKALIYSASFRYS |
| | | GVPSRFSGSGSGTDFTLTISSLQPEDFATYYCQQYYTYPYTFGGGTKVEIK** |
| 285 | ACP455 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpALFKSSFPpgsEVQLVESGGGLVQ |
| | | PGNSLRLSCAASGFTFSKFGMSWVRQAPGKGLEWVSSISGSGRDTLYAESVKGR |
| | | FTISRDNAKTTLYLQMNSLRPEDTAVYYCTIGGSLSVSSQGTLVTVSSggggsggggs |
| | | ggggsggggsggggsggggssggpALFKSSFPpgsEVQLVESGGGLVQPGGSLRLSCAASGF |
| | | TFSSYTLAWVRQAPGKcLEWVAAIDSSSYTYSPDTVRGRFTISRDNAKNSLYLQ |
| | | MNSLRAEDTAVYYCARDSNWDALDYWGQGTTVTVSSGGGGSGGGGSGGGGS |
| | | DIQMTQSPSSLSASVGDRVTITCKVTEKVWGNVAWYQQKPGKAPISLIYSPSLRK |
| | | SGVPSRFSGSGSGTDFTLTISSLQPEDFATYYCQQYYTYPYTFGcGTKVEIK** |
| 441 | ACP456 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpALFKSSFPpgsEVQLVESGGGLVQ |
| | | PGNSLRLSCAASGFTFSKFGMSWVRQAPGKGLEWVSSISGSGRDTLYAESVKGR |
| | | FTISRDNAKTTLYLQMNSLRPEDTAVYYCTIGGSLSVSSQGTLVTVSSggggsggggs |
| | | ggggsggggsggggsggggssggpALFKSSFPpgsEVQLVESGGGLVQPGGSLRLSCAASGF |
| | | TFSSYTLAWVRQAPGKGLEWVAAIDSSSYTYSPDTVRGRFTISRDNAKNSLYLQ |
| | | MNSLRAEDTAVYYCARDSNWDALDYWGcGTTVTVSSGGGGSGGGGSGGGGSD |
| | | IQMTQSPSSLSASVGDRVTITCKVTEKVWGNVAWYQQKPGKcPISLIYSPSLRKS |
| | | GVPSRFSGSGSGTDFTLTISSLQPEDFATYYCQQYYTYPYTFGGGTKVEIK** |
| 286 | ACP457 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpALFKSSFPpgsEVQLVESGGGLVQ |
| | | PGNSLRLSCAASGFTFSKFGMSWVRQAPGKGLEWVSSISGSGRDTLYAESVKGR |
| | | FTISRDNAKTTLYLQMNSLRPEDTAVYYCTIGGSLSVSSQGTLVTVSSggggsggggs |
| | | ggggsggggsggggsggggssggpALFKSSFPpgsEVQLVESGGGLVQPGGSLRLSCAASGF |
| | | TFSSYTLAWVRQAPGKGLEWVAAIDSSSYTYSPDTVRGRFTISRDNAKNSLYLQ |
| | | MNSLRAEDTAVYYCARDSNWDALDYWGQGTTVTVSSastkgpsvfplapsskstsggtaalg |
| | | clvkdyfpepvtvswnsgaltsgvhtfpavlqssglyslssvvtvpssslgtqtyicnvnhkpsntkvdkrvepksc** |
| 287 | ACP458 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpALFK |
| | | SSFPpgsaptssstkktqlqlehllldlqmilnginnyknpkltrmltfkfympkkatelkhlqcleeelkpleevlnlaqsk |
| | | nfhlrprdlisninvivlelkgsettfmceyadetativeflnrwitfcqsiistltggggsggggsggggsggggsggggsgg |
| | | ggssggpALFK SSFPpgsEVQLVESGGGLVQPGGSLRLSCAASGFTFSSYTLAWVRQ |
| | | APGKGLEWVAAIDSSSYTYSPDTVRGRFTISRDNAKNSLYLQMNSLRAEDTAVY |
| | | YCARDSNWDALDYWGQGTTVTVSSastkgpsvfplapsskstsggtaalgclvkdyfpepvtvswnsg |
| | | altsgvhtfpavlqssglyslssvvtvpssslgtqtyicnvnhkpsntkvdkrvepksc** |
| 288 | ACP459 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpALFK |
| | | SSFPpgsaptssstkktqlqlehllldlqmilnginnyknpkltrmltfkfympkkatelkhlqcleeelkpleevlnlaqsk |
| | | nfhlrprdlisninvivlelkgsettfmceyadetativeflnrwitfcqsiistltggggsggggsggggsggggsggggsgg |
| | | ggssggpALFKSSFPpgsEVQLVESGGGLVQPGGSLRLSCAASGFTFSSYTLAWVRQ |
| | | APGKGLEWVAAIDSSSYTYSPDTVRGRFTISRDNAKNSLYLQMNSLRAEDTAVY |
| | | YCARDSNWDALDYWGQGTTVTVSSGGGGSGGGGSGGGGSDIQMTQSPSSLSAS |
| | | VGDRVTITCKAREKLWSAVAWYQQKPGKAPKsLIYSASFRYSGVPSRFSGSGSG |
| | | TDFTLTISSLQPEDFATYYCQQYYTYPYTFGGGTKVEIK** |
| 289 | ACP460 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpALFK |
| | | SSFPpgsaptssstkktqlqlehllldlqmilnginnyknpkltrmltfkfympkkatelkhlqcleeelkpleevlnlaqsk |
| | | nfhlrprdlisninvivlelkgsettfmceyadetativeflnrwitfcqsiistltggggsggggsggggsggggsggggsgg |
| | | ggssggpALFKSSFPpgsEVQLVESGGGLVQPGGSLRLSCAASGFTFSSYTLAWVRQ |
| | | APGKGLEWVAAIDSSSYTYSPDTVRGRFTISRDNAKNSLYLQMNSLRAEDTAVY |
| | | YCARDSNWDALDYWGQGTTVTVSSGGGGSGGGGSGGGGSDIQMTQSPSSLSAS |
| | | VGDRVTITCKVTEKVWGNVAWYQQKPGKAPISLIYSPSLRKSGVPSRFSGSGSG |
| | | TDFTLTISSLQPEDFATYYCQQYYTYPYTFGGGTKVEIK** |
| 290 | ACP461 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpALFK |
| | | SSFPpgsaptssstkktqlqlehllldlqmilnginnyknpkltrmltfkfympkkatelkhlqcleeelkpleevlnlaqsk |
| | | nfhlrprdlisninvivlelkgsettfmceyadetativeflnrwitfcqsiistltggggsggggsggggsggggsggggsgg |
| | | ggssggpALFKSSFPpgsEVQLVESGGGLVQPGGSLRLSCAASGFTFSSYTLAWVRQ |
| | | APGKcLEWVAAIDSSSYTYSPDTVRGRFTISRDNAKNSLYLQMNSLRAEDTAVY |
| | | YCARDSNWDALDYWGQGTTVTVSSGGGGSGGGGSGGGGSDIQMTQSPSSLSAS |
| | | VGDRVTITCKAREKLWSAVAWYQQKPGKAPKsLIYSASFRYSGVPSRFSGSGSG |
| | | TDFTLTISSLQPEDFATYYCQQYYTYPYTFGcGTKVEIK** |
| 291 | ACP462 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpALFK |
| | | SSFPpgsaptssstkktqlqlehllldlqmilnginnyknpkltrmltfkfympkkatelkhlqcleeelkpleevlnlaqsk |
| | | nfhlrprdlisninvivlelkgsettfmceyadetativeflnrwitfcqsiistltggggsggggsggggsggggsggggsgg |
| | | ggssggpALFKSSFPpgsEVQLVESGGGLVQPGGSLRLSCAASGFTFSSYTLAWVRQ |
| | | APGKGLEWVAAIDSSSYTYSPDTVRGRFTISRDNAKNSLYLQMNSLRAEDTAVY |
| | | YCARDSNWDALDYWGcGTTVTVSSGGGGSGGGGSGGGGSDIQMTQSPSSLSAS |
| | | VGDRVTITCKAREKLWSAVAWYQQKPGKcPKALIYSASFRYSGVPSRFSGSGSG |
| | | TDFTLTISSLQPEDFATYYCQQYYTYPYTFGGGTKVEIK** |
| 292 | ACP463 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpALFK |
| | | SSFPpgsaptssstkktqlqlehllldlqmilnginnyknpkltrmltfkfympkkatelkhlqcleeelkpleevlnlaqsk |
| | | nfhlrprdlisninvivlelkgsettfmceyadetativeflnrwitfcqsiistltggggsggggsggggsggggsggggsgg |
| | | ggssggpALFKSSFPpgsEVQLVESGGGLVQPGGSLRLSCAASGFTFSSYTLAWVRQ |
| | | APGKcLEWVAAIDSSSYTYSPDTVRGRFTISRDNAKNSLYLQMNSLRAEDTAVY |
| | | YCARDSNWDALDYWGQGTTVTVSSGGGGSGGGGSGGGGSDIQMTQSPSSLSAS |
| | | VGDRVTITCKVTEKVWGNVAWYQQKPGKAPISLIYSPSLRKSGVPSRFSGSGSG |
| | | TDFTLTISSLQPEDFATYYCQQYYTYPYTFGcGTKVEIK** |
| 293 | ACP464 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpALFK |
| | | SSFPpgsaptssstkktqlqlehllldlqmilnginnyknpkltrmltfkfympkkatelkhlqcleeelkpleevlnlaqsk |
| | | nfhlrprdlisninvivlelkgsettfmceyadetativeflnrwitfcqsiistltggggsggggsggggsggggsggggsgg |
| | | ggssggpALFKSSFPpgsEVQLVESGGGLVQPGGSLRLSCAASGFTFSSYTLAWVRQ |
| | | APGKGLEWVAAIDSSSYTYSPDTVRGRFTISRDNAKNSLYLQMNSLRAEDTAVY |
| | | YCARDSNWDALDYWGcGTTVTVSSGGGGSGGGGSGGGGSDIQMTQSPSSLSAS |
| | | VGDRVTITCKVTEKVWGNVAWYQQKPGKcPISLIYSPSLRKSGVPSRFSGSGSGT |
| | | DFTLTISSLQPEDFATYYCQQYYTYPYTFGGGTKVEIK** |
| 294 | ACP465 | vprdcgckpcictypevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnvqksnweagntftcsvlheglhnhhtekslshspgksggpALFKSSF |
| | | Ppgsaptssstkktqlqlehllldlqmilnginnyknpkltrmltfkfympkkatelkhlqcleeelkpleevlnlaqsknfhlr |
| | | prdlisninvivlelkgsettfmceyadetativeflnrwitfcqsiistltggggsggggsggggsggggsggggsggggssg |
| | | gpALFKSSFPpgsEVQLVESGGGLVQPGGSLRLSCAASGFTFSSYTLAWVRQAPGK |
| | | GLEWVAAIDSSSYTYSPDTVRGRFTISRDNAKNSLYLQMNSLRAEDTAVYYCAR |
| | | DSNWDALDYWGQGTTVTVSSGGGGSGGGGSGGGGSDIQMTQSPSSLSASVGDR |
| | | VTITCKAREKLWSAVAWYQQKPGKAPKsLIYSASFRYSGVPSRFSGSGSGTDFTL |
| | | TISSLQPEDFATYYCQQYYTYPYTFGGGTKVEIK** |
| 295 | ACP466 | vprdcgckpcictypevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfyysklnvqksnweagntftcsvlheglhnhhtekslshspgksggpALFKSSF |
| | | Ppgsaptssstkktqlqlehllldlqmilnginnyknpkltrmltfkfympkkatelkhlqcleeelkpleevlnlaqsknfhlr |
| | | prdlisninvivlelkgsettfmceyadetativeflnrwitfcqsiistltggggsggggsggggsggggsggggsggggssg |
| | | gpALFKSSFPpgsEVQLVESGGGLVQPGGSLRLSCAASGFTFSSYTLAWVRQAPGK |
| | | GLEWVAAIDSSSYTYSPDTVRGRFTISRDNAKNSLYLQMNSLRAEDTAVYYCAR |
| | | DSNWDALDYWGQGTTVTVSSGGGGSGGGGSGGGGSDIQMTQSPSSLSASVGDR |
| | | VTITCKVTEKVWGNVAWYQQKPGKAPISLIYSPSLRKSGVPSRFSGSGSGTDFTL |
| | | TISSLQPEDFATYYCQQYYTYPYTFGGGTKVEIK** |
| 296 | ACP467 | vprdcgckpcictypevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnvqksnweagntftcsvlheglhnhhtekslshspgksggpALFKSSF |
| | | Ppgsaptssstkktqlqlehllldlqmilnginnyknpkltrmltfkfympkkatelkhlqcleeelkpleevlnlaqsknfhlr |
| | | prdlisninvivlelkgsettfmceyadetativeflnrwitfcqsiistltggggsggggsggggsggggsggggsggggssg |
| | | gpALFKSSFPpgsEVQLVESGGGLVQPGGSLRLSCAASGFTFSSYTLAWVRQAPGK |
| | | cLEWVAAIDSSSYTYSPDTVRGRFTISRDNAKNSLYLQMNSLRAEDTAVYYCAR |
| | | DSNWDALDYWGQGTTVTVSSGGGGSGGGGSGGGGSDIQMTQSPSSLSASVGDR |
| | | VTITCKAREKLW SAVAWYQQKPGKAPKsLIYSASFRYSGVPSRFSGSGSGTDFTL |
| | | TISSLQPEDFATYYCQQYYTYPYTFGcGTKVEIK** |
| 297 | ACP468 | vprdcgckpcictvpevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnvqksnweagntftcsvlheglhnhhtekslshspgksggpALFKSSF |
| | | Ppgsaptssstkktqlqlehllldlqmilnginnyknpkltrmltfkfympkkatelkhlqcleeelkpleevlnlaqsknfhlr |
| | | prdlisninvivlelkgsettfmceyadetativeflnrwitfcqsiistltggggsggggsggggsggggsggggsggggssg |
| | | gpALFKSSFPpgsEVQLVESGGGLVQPGGSLRLSCAASGFTFSSYTLAWVRQAPGK |
| | | GLEWVAAIDSSSYTYSPDTVRGRFTISRDNAKNSLYLQMNSLRAEDTAVYYCAR |
| | | DSNWDALDYWGcGTTVTVSSGGGGSGGGGSGGGGSDIQMTQSPSSLSASVGDR |
| | | VTITCKAREKLW SAVAWYQQKPGKcPKALIYSASFRYSGVPSRFSGSGSGTDFTL |
| | | TISSLQPEDFATYYCQQYYTYPYTFGGGTKVEIK** |
| 298 | ACP469 | vprdcgckpcictvpevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnvqksnweagntftcsvlheglhnhhtekslshspgksggpALFKSSF |
| | | Ppgsaptssstkktqlqlehllldlqmilnginnyknpkltrmltfkfympkkatelkhlqcleeelkpleevlnlaqsknfhlr |
| | | prdlisninvivlelkgsettfmceyadetativeflnrwitfcqsiistltggggsggggsggggsggggsggggsggggssg |
| | | gpALFKSSFPpgsEVQLVESGGGLVQPGGSLRLSCAASGFTFSSYTLAWVRQAPGK |
| | | cLEWVAAIDSSSYTYSPDTVRGRFTISRDNAKNSLYLQMNSLRAEDTAVYYCAR |
| | | DSNWDALDYWGQGTTVTVSSGGGGSGGGGSGGGGSDIQMTQSPSSLSASVGDR |
| | | VTITCKVTEKVWGNVAWYQQKPGKAPISLIYSPSLRKSGVPSRFSGSGSGTDFTL |
| | | TISSLQPEDFATYYCQQYYTYPYTFGcGTKVEIK** |
| 299 | ACP470 | vprdcgckpcictvpevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnvqksnweagntftcsvlheglhnhhtekslshspgksggpALFKSSF |
| | | Ppgsaptssstkktqlqlehllldlqmilnginnyknpkltrmltfkfympkkatelkhlqcleeelkpleevlnlaqsknfhlr |
| | | prdlisninvivlelkgsettfmceyadetativeflnrwitfcqsiistltggggsggggsggggsggggsggggsggggssg |
| | | gpALFKSSFPpgsEVQLVESGGGLVQPGGSLRLSCAASGFTFSSYTLAWVRQAPGK |
| | | GLEWVAAIDSSSYTYSPDTVRGRFTISRDNAKNSLYLQMNSLRAEDTAVYYCAR |
| | | DSNWDALDYWGcGTTVTVSSGGGGSGGGGSGGGGSDIQMTQSPSSLSASVGDR |
| | | VTITCKVTEKVWGNVAWYQQKPGKcPISLIYSPSLRKSGVPSRFSGSGSGTDFTLT |
| | | ISSLQPEDFATYYCQQYYTYPYTFGGGTKVEIK** |
| 300 | ACP471 | mdmrvpaqllgllllwlrgarcvprdcgckpcictvpevssvfifppkpkdvltitltpkvtcvvvdiskddpevqfswfvd |
| | | dvevhtaqtqpreeqfnstfrsyselpimhqdwlngkefkcrvnsaafpapiektisktkgrpkapqvytipppkeqmak |
| | | dkvsltcmitdffpeditvewqwngqpaenykntqpimdtdgsyfvysklnvqksnweagntftcsvlheglhnhhtek |
| | | slshspgksggpALFKSSFPpgsaptssstkktqlqlehllldlqmilnginnyknpkltrmltfkfympkkatelkhlq |
| | | cleeelkpleevlnlaqsknfhlrprdlisninvivlelkgsettfmceyadetativeflnrwitfcqsiistltggggsggggsg |
| | | gggsggggsggggsggggssggpALFKSSFPpgsEVQLVESGGGLVQPGGSLRLSCAASGFT |
| | | FSSYTLAWVRQAPGKGLEWVAAIDSSSYTYSPDTVRGRFTISRDNAKNSLYLQM |
| | | NSLRAEDTAVYYCARDSNWDALDYWGQGTTVTVSSastkgpsvfplapsskstsggtaalgcl |
| | | vkdyfpepvtvswnsgaltsgvhtfpavlqssglyslssvvtvpssslgtqtyicnvnhkpsntkvdkrvepksc** |
| 301 | ACP382 | DIQMTQSPSSLSASVGDRVTITCKVTEKVWGNVAWYQQKPGKAPISLIYS |
| | | PSLRKSGVPSRFSGSGSGTDFTLTISSLQPEDFATYYCQQYYTYPYTFGGG |
| | | TKVEIKrtvaapsvfifppsdeqlksgtasvvcllnnfypreakvqwkvdnalqsgnsqesvteqdskdsty |
| | | slssthlskadyekhkvyacevthqglsspvtksfnrgec** |
| 302 | ACP383 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKcLEWVAAIDSSSYTYSPDTVRGRFTISRDNAKNSLYLQMNSLRAEDTAV |
| | | YYCARDSNWDALDYWGQGTTVTVSSGGGGSGGGGSGGGGSDIQMTQSPSSLS |
| | | ASVGDRVTITCKASQNVGTNVGWYQQKPGKAPKALIYSASFRYSGVPSRFSGSG |
| | | SGTDFTLTISSLQPEDFATYYCQQYYTYPYTFGcGTKVEIK** |
| 303 | ACP384 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKGLEWVAAIDSSSYTYSPDTVRGRFTISRDNAKNSLYLQMNSLRAEDTAV |
| | | YYCARDSNWDALDYWGcGTTVTVSSGGGGSGGGGSGGGGSDIQMTQSPSSLSA |
| | | SVGDRVTITCKASQNVGTNVGWYQQKPGKcPKALIYSASFRYSGVPSRFSGSGS |
| | | GTDFTLTISSLQPEDFATYYCQQYYTYPYTFGGGTKVEIK** |
| 304 | ACP385 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKcLEWVAAIDSSSYTYSPDTVRGRFTISRDNAKNSLYLQMNSLRAEDTAV |
| | | YYCARDSNWDALDYWGQGTTVTVSSGGGGSGGGGSGGGGSDIQMTQSPSSLS |
| | | ASVGDRVTITCKAREKLW SAVAWYQQKPGKAPKALIYSASFRYSGVPSRFSGSG |
| | | SGTDFTLTISSLQPEDFATYYCQQYYTYPYTFGcGTKVEIK** |
| 305 | ACP386 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKGLEWVAAIDSSSYTYSPDTVRGRFTISRDNAKNSLYLQMNSLRAEDTAV |
| | | YYCARDSNWDALDYWGcGTTVTVSSGGGGSGGGGSGGGGSDIQMTQSPSSLSA |
| | | SVGDRVTITCKAREKLWSAVAWYQQKPGKcPKALIYSASFRYSGVPSRFSGSGS |
| | | GTDFTLTISSLQPEDFATYYCQQYYTYPYTFGGGTKVEIK** |
| 306 | ACP387 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKcLEWVAAIDSSSYTYSPDTVRGRFTISRDNAKNSLYLQMNSLRAEDTAV |
| | | YYCARDSNWDALDYWGQGTTVTVSSGGGGSGGGGSGGGGSDIQMTQSPSSLS |
| | | ASVGDRVTITCKVTEKVWGNVAWYQQKPGKAPISLIYSPSLRKSGVPSRFSGSGS |
| | | GTDFTLTISSLQPEDFATYYCQQYYTYPYTFGcGTKVEIK** |
| 307 | ACP388 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKGLEWVAAIDSSSYTYSPDTVRGRFTISRDNAKNSLYLQMNSLRAEDTAV |
| | | YYCARDSNWDALDYWGcGTTVTVSSGGGGSGGGGSGGGGSDIQMTQSPSSLSA |
| | | SVGDRVTITCKVTEKVWGNVAWYQQKPGKcPISLIYSPSLRKSGVPSRFSGSGSG |
| | | TDFTLTISSLQPEDFATYYCQQYYTYPYTFGGGTKVEIK** |
| 308 | ACP389 | eskygppcppcpapeflggpsvflfppkpkdtlmisrtpevtcyvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsevqllesggglvqpggslrlscaasgsifsanamgwyrqapgkqrelvavissggst |
| | | nyadsvkgrftisrdnskntvylqmnslraedtavyycmysgsyyytpndywgqgtlvtvss** |
| 309 | ACP390 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKcLEWVAAIDSSSYTYSPDTVRGRFTISRDNAKNSLYL |
| | | QMNSLRAEDTAVYYCARDSNWDALDYWGQGTTVTVSSGGGGSGGGGSGGGG |
| | | SDIQMTQSPSSLSASVGDRVTITCKAREKLWSAVAWYQQKPGKAPKsLIYSASFR |
| | | YSGVPSRFSGSGSGTDFTLTISSLQPEDFATYYCQQYYTYPYTFGcGTKVEIK** |
| 310 | ACP391 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKcLEWVAAIDSSSYTYSPDTVRGRFTISRDNAKNSLYLQMNSLRAEDTAV |
| | | YYCARDSNWDALDYWGQGTTVTVSSGGGGSGGGGSGGGGSDIQMTQSPSSLS |
| | | ASVGDRVTITCKAREKLWSAVAWYQQKPGKAPKsLIYSASFRYSGVPSRFSGSG |
| | | SGTDFTLTISSLQPEDFATYYCQQYYTYPYTFGcGTKVEIK** |
| 311 | ACP392 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggselcdddppeiphatfkamaykegtmlnceckrgfrriksgslymlctgnsshsswdnqcqctssatr |
| | | nttkqvtpqpeeqkerkttemqspmqpvdqaslpghcrepppweneateriyhfvvgqmvyyqcvqgyralhrgpae |
| | | svckmthgktrwtqpqlictgemetsqfpgeekpqaspegrpesetsSlvtttdfqiqtemaatmetsiftteyqsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsEVQL |
| | | VESGGGLVQPGGSLRLSCAASGFTFSSYTLAWVRQAPGKcLEWVAAIDSSSYTY |
| | | SPDTVRGRFTISRDNAKNSLYLQMNSLRAEDTAVYYCARDSNWDALDYWGQG |
| | | TTVTVSSGGGGSGGGGSGGGGSDIQMTQSPSSLSASVGDRVTITCKAREKLWSA |
| | | VAWYQQKPGKAPKsLIYSASFRYSGVPSRFSGSGSGTDFTLTISSLQPEDFATYYC |
| | | QQYYTYPYTFGcGTKVEIK** |
| 312 | ACP393 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggselcdddppeiphatfkamaykegtmlnceckrgfrriksgslymlctgnsshsswdnqcqctssatr |
| | | nttkqvtpqpeeqkerkttemqspmqpvdqaslpghcrepppweneateriyhfvvgqmvyyqcvqgyralhrgpae |
| | | svckmthgktrwtqpqlictgemetsqfpgeekpqaspegrpesetsSlvtttdfqiqtemaatmetsiftteyqsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsEVQL |
| | | VESGGGLVQPGGSLRLSCAASGFTFSSYTLAWVRQAPGKGLEWVAAIDSSSYTY |
| | | SPDTVRGRFTISRDNAKNSLYLQMNSLRAEDTAVYYCARDSNWDALDYWGcGT |
| | | TVTVSSGGGGSGGGGSGGGGSDIQMTQSPSSLSASVGDRVTITCKAREKLWSAV |
| | | AWYQQKPGKcPKALIYSASFRYSGVPSRFSGSGSGTDFTLTISSLQPEDFATYYCQ |
| | | QYYTYPYTFGGGTKVEIK** |
| 313 | ACP394 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggselcdddppeiphatfkamaykegtmlnceckrgfrriksgslymlctgnsshsswdnqcqctssatr |
| | | nttkqvtpqpeeqkerkttemqspmqpvdqaslpghcrepppweneateriyhfvvgqmvyyqcvqgyralhrgpae |
| | | svckmthgktrwtqpqlictgemetsqfpgeekpqaspegrpesetsSlvtttdfqiqtemaatmetsiftteyqsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsEVQL |
| | | VESGGGLVQPGGSLRLSCAASGFTFSSYTLAWVRQAPGKcLEWVAAIDSSSYTY |
| | | SPDTVRGRFTISRDNAKNSLYLQMNSLRAEDTAVYYCARDSNWDALDYWGQG |
| | | TTVTVSSGGGGSGGGGSGGGGSDIQMTQSPSSLSASVGDRVTITCKVTEKVWGN |
| | | VAWYQQKPGKAPISLIYSPSLRKSGVPSRFSGSGSGTDFTLTISSLQPEDFATYYC |
| | | QQYYTYPYTFGcGTKVEIK** |
| 314 | ACP395 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggselcdddppeiphatfkamaykegtmlnceckrgfrriksgslymlctgnsshsswdnqcqctssatr |
| | | nttkqvtpqpeeqkerkttemqspmqpvdqaslpghcrepppweneateriyhfvvgqmvyyqcvqgyralhrgpae |
| | | svckmthgktrwtqpqlictgemetsqfpgeekpqaspegrpesetsSlvtttdfqiqtemaatmetsiftteyqsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsEVQL |
| | | VESGGGLVQPGGSLRLSCAASGFTFSSYTLAWVRQAPGKGLEWVAAIDSSSYTY |
| | | SPDTVRGRFTISRDNAKNSLYLQMNSLRAEDTAVYYCARDSNWDALDYWGcGT |
| | | TVTVSSGGGGSGGGGSGGGGSDIQMTQSPSSLSASVGDRVTITCKVTEKVWGNV |
| | | AWYQQKPGKcPISLIYSPSLRKSGVPSRFSGSGSGTDFTLTISSLQPEDFATYYCQQ |
| | | YYTYPYTFGGGTKVEIK** |
| 315 | ACP396 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggselcdddppeiphatfkamaykegtmlnceckrgfrriksgslymlctgnsshsswdnqcqctssatr |
| | | nttkqvtpqpeeqkerkttemqspmqpvdqaslpghcrepppweneateriyhfvvgqmvyyqcvqgyralhrgpae |
| | | svckmthgktrwtqpqlictgemetsqfpgeekpqaspegrpesetsSlvtttdfqiqtemaatmetsiftteyqsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsevqlles |
| | | ggglvqpggslrlscaasgsifsanamgwyrqapgkqrelvavissggstnyadsvkgrftisrdnskntvylqmnslraed |
| | | tavyycmysgsyyytpndywgqgtlvtvss** |
| 316 | ACP397 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggselcdddppeiphatfkamaykegtmlnceckrgfrriksgslymlctgnsshsswdnqcqctssatr |
| | | nttkqvtpqpeeqkerkttemqspmqpvdqaslpghcrepppweneateriyhfvvgqmvyyqcvqgyralhrgpae |
| | | svckmthgktrwtqpqlictgemetsqfpgeekpqaspegrpesetsSlvtttdfqiqtemaatmetsiftteyqsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsevqlles |
| | | ggglvqpggslrlscaasgsifsanamgwyrqapgkglelvavissggstnyadsvkgrftisrdnskntvylqmnslraed |
| | | tavyycmysgsyyytpndywgqgtlvtvss** |
| 317 | ACP398 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggselcdddppeiphatfkamaykegtmlnceckrgfrriksgslymlctgnsshsswdnqcqctssatr |
| | | nttkqvtpqpeeqkerkttemqspmqpvdqaslpghcrepppweneateriyhfvvgqmvyyqcvqgyralhrgpae |
| | | svckmthgktrwtqpqlictgemetsqfpgeekpqaspegrpesetsSlvtttdfqiqtemaatmetsiftteyqsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsEVQL |
| | | VESGGGLVQPGGSLRLSCAASGFTFSSYTLAWVRQAPGKGLEWVAAIDSSSYTY |
| | | SPDTVRGRFTISRDNAKNSLYLQMNSLRAEDTAVYYCARDSNWDALDYWGQG |
| | | TTVTVSSastkgpsvfplapsskstsggtaalgclvkdyfpepvtvswnsgaltsgvhtfpavlqssglyslssvvtvpss |
| | | slgtqtyicnvnhkpsntkvdkrvepksc** |
| 318 | ACP399 | EVQLVESGGGLVQPGGSLRLSCAASGFTFSSYTLAWVRQAPGKcLEWVAAIDSS |
| | | SYTYSPDTVRGRFTISRDNAKNSLYLQMNSLRAEDTAVYYCARDSNWDALDYW |
| | | GQGTTVTVSSGGGGSGGGGSGGGGSDIQMTQSPSSLSASVGDRVTITCKAREKL |
| | | WSAVAWYQQKPGKAPKsLIYSASFRYSGVPSRFSGSGSGTDFTLTISSLQPEDFAT |
| | | YYCQQYYTYPYTFGcGTKVEIKsggpGPAGLYAQpgsggggsggggsggggsggggsggggsg |
| | | gggsEVQLVESGGGLVQPGNSLRLSCAASGFTFSKFGMSWVRQAPGKGLEWVSSI |
| | | SGSGRDTLYAESVKGRFTISRDNAKTTLYLQMNSLRPEDTAVYYCTIGGSLSVSS |
| | | QGTLVTVSSsggpGPAGLYAQpgstfkfympkkatelkhlqcleeelkpleevlnlaqsknfhlrprdlisnin |
| | | vivlelkgsettfmceyadetativeflnrwitfcqsiistltGGssstkktqlqlehllldlqmilnginnyknpkltrmlsggp |
| | | GPAGLYAQpgsEVQLVESGGGLVQPGNSLRLSCAASGFTFSKFGMSWVRQAPGK |
| | | GLEWVSSISGSGRDTLYAESVKGRFTISRDNAKTTLYLQMNSLRPEDTAVYYCTI |
| | | GGSLSVSSQGTLVTVSS** |
| 319 | ACP400 | EVQLVESGGGLVQPGGSLRLSCAASGFTFSSYTLAWVRQAPGKGLEWVAAIDSS |
| | | SYTYSPDTVRGRFTISRDNAKNSLYLQMNSLRAEDTAVYYCARDSNWDALDYW |
| | | GcGTTVTVSSGGGGSGGGGSGGGGSDIQMTQSPSSLSASVGDRVTITCKAREKL |
| | | WSAVAWYQQKPGKcPKALIYSASFRYSGVPSRFSGSGSGTDFTLTISSLQPEDFA |
| | | TYYCQQYYTYPYTFGGGTKVEIKsggpGPAGLYAQpgsggggsggggsggggsggggsgggg |
| | | sggggsEVQLVESGGGLVQPGNSLRLSCAASGFTFSKFGMSWVRQAPGKGLEWVS |
| | | SISGSGRDTLYAESVKGRFTISRDNAKTTLYLQMNSLRPEDTAVYYCTIGGSLSV |
| | | SSQGTLVTVSSsggpGPAGLYAQpgstfkfympkkatelkhlqcleeelkpleevlnlaqsknfhlrprdlisn |
| | | invivlelkgsettfmceyadetativeflnrwitfcqsiistltGGssstkktqlqlehllldlqmilnginnyknpkltrmlsg |
| | | gpGPAGLYAQpgsEVQLVESGGGLVQPGNSLRLSCAASGFTFSKFGMSWVRQAP |
| | | GKGLEWVSSISGSGRDTLYAESVKGRFTISRDNAKTTLYLQMNSLRPEDTAVYY |
| | | CTIGGSLSVSSQGTLVTVSS** |
| 320 | ACP401 | EVQLVESGGGLVQPGGSLRLSCAASGFTFSSYTLAWVRQAPGKcLEWVAAIDSS |
| | | SYTYSPDTVRGRFTISRDNAKNSLYLQMNSLRAEDTAVYYCARDSNWDALDYW |
| | | GQGTTVTVSSGGGGSGGGGSGGGGSDIQMTQSPSSLSASVGDRVTITCKVTEKV |
| | | WGNVAWYQQKPGKAPISLIYSPSLRKSGVPSRFSGSGSGTDFTLTISSLQPEDFAT |
| | | YYCQQYYTYPYTFGcGTKVEIKsggpGPAGLYAQpgsggggsggggsggggsggggsggggsg |
| | | gggsEVQLVESGGGLVQPGNSLRLSCAASGFTFSKFGMSWVRQAPGKGLEWVSSI |
| | | SGSGRDTLYAESVKGRFTISRDNAKTTLYLQMNSLRPEDTAVYYCTIGGSLSVSS |
| | | QGTLVTVSSsggpGPAGLYAQpgstfkfympkkatelkhlqcleeelkpleevlnlaqsknfhlrprdlisnin |
| | | vivlelkgsettfmceyadetativeflnrwitfcqsiistltGGssstkktqlqlehllldlqmilnginnyknpkhrmlsggp |
| | | GPAGLYAQpgsEVQLVESGGGLVQPGNSLRLSCAASGFTFSKFGMSWVRQAPGK |
| | | GLEWVSSISGSGRDTLYAESVKGRFTISRDNAKTTLYLQMNSLRPEDTAVYYCTI |
| | | GGSLSVSSQGTLVTVSS** |
| 321 | ACP402 | EVQLVESGGGLVQPGGSLRLSCAASGFTFSSYTLAWVRQAPGKGLEWVAAIDSS |
| | | SYTYSPDTVRGRFTISRDNAKNSLYLQMNSLRAEDTAVYYCARDSNWDALDYW |
| | | GcGTTVTVSSGGGGSGGGGSGGGGSDIQMTQSPSSLSASVGDRVTITCKVTEKV |
| | | WGNVAWYQQKPGKcPISLIYSPSLRKSGVPSRFSGSGSGTDFTLTISSLQPEDFAT |
| | | YYCQQYYTYPYTFGGGTKVEIKsggpGPAGLYAQpgsggggsggggsggggsggggsggggs |
| | | ggggsEVQLVESGGGLVQPGNSLRLSCAASGFTFSKFGMSWVRQAPGKGLEWVSS |
| | | ISGSGRDTLYAESVKGRFTISRDNAKTTLYLQMNSLRPEDTAVYYCTIGGSLSVS |
| | | SQGTLVTVSSsggpGPAGLYAQpgstfkfympkkatelkhlqcleeelkpleevlnlaqsknfhlrprdlisni |
| | | nvivlelkgsettfmceyadetativeflnrwitfcqsiistltGGssstkktqlqlehllldlqmilnginnyknpkltrmlsgg |
| | | pGPAGLYAQpgsEVQLVESGGGLVQPGNSLRLSCAASGFTFSKFGMSWVRQAPG |
| | | KGLEWVSSISGSGRDTLYAESVKGRFTISRDNAKTTLYLQMNSLRPEDTAVYYC |
| | | TIGGSLSVSSQGTLVTVSS** |
| 322 | ACP403 | evqllesggglvqpggslrlscaasgsifsanamgwyrqapgkqrelvavissggstnyadsvkgrftisrdnskntvylqm |
| | | nslraedtavyycmysgsyyytpndywgqgtlvtvsssggpGPAGLYAQpgsggggsggggsggggsggggsg |
| | | gggsggggsEVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVYYCTIGGS |
| | | LSVSSQGTLVTVSSsggpGPAGLYAQpgstfkfympkkatelkhlqcleeelkpleevlnlaqsknfhlrpr |
| | | dlisninvivlelkgsettfmceyadetativeflnrwitfcqsiistltGGssstkktqlqlehllldlqmilnginnyknpkltr |
| | | mlsggpGPAGLYAQpgsEVQLVESGGGLVQPGNSLRLSCAASGFTFSKFGMSWVRQ |
| | | APGKGLEWVSSISGSGRDTLYAESVKGRFTISRDNAKTTLYLQMNSLRPEDTAV |
| | | YYCTIGGSLSVSSQGTLVTVSS** |
| 323 | ACP404 | evqllesggglvqpggslrlscaasgsifsanamgwyrqapgkglelvavissggstnyadsvkgrftisrdnskntvylqm |
| | | nslraedtavyycmysgsyyytpndywgqgtlvtvsssggpGPAGLYAQpgsggggsggggsggggsggggsg |
| | | gggsggggsEVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVYYCTIGGS |
| | | LSVSSQGTLVTVSSsggpGPAGLYAQpgstfkfympkkatelkhlqcleeelkpleevlnlaqsknfhlrpr |
| | | dlisninvivlelkgsettfmceyadetativeflnrwitfcqsiistltGGssstkktqlqlehllldlqmilnginnyknpkltr |
| | | mlsggpGPAGLYAQpgsEVQLVESGGGLVQPGNSLRLSCAASGFTFSKFGMSWVRQ |
| | | APGKGLEWVSSISGSGRDTLYAESVKGRFTISRDNAKTTLYLQMNSLRPEDTAV |
| | | YYCTIGGSLSVSSQGTLVTVSS** |
| 324 | ACP405 | EVQLVESGGGLVQPGGSLRLSCAASGFTFSSYTLAWVRQAPGKGLEWVAAIDSS |
| | | SYTYSPDTVRGRFTISRDNAKNSLYLQMNSLRAEDTAVYYCARDSNWDALDYW |
| | | GQGTTVTVSSastkgpsvfplapsskstsggtaalgclvkdyfpepvtvswnsgaltsgvhtfpavlqssglyslssv |
| | | vtvpssslgtqtyicnvnhkpsntkvdkrvepkscsggpGPAGLYAQpgsggggsggggsggggsggggsgggg |
| | | sggggsEVQLVESGGGLVQPGNSLRLSCAASGFTFSKFGMSWVRQAPGKGLEWVS |
| | | SISGSGRDTLYAESVKGRFTISRDNAKTTLYLQMNSLRPEDTAVYYCTIGGSLSV |
| | | SSQGTLVTVSSsggpGPAGLYAQpgstfkfympkkatelkhlqcleeelkpleevlnlaqsknfhlrprdlisn |
| | | invivlelkgsettfmceyadetativeflnrwitfcqsiistltGGssstkktqlqlehllldlqmilnginnyknpkltrmlsg |
| | | gpGPAGLYAQpgsEVQLVESGGGLVQPGNSLRLSCAASGFTFSKFGMSWVRQAP |
| | | GKGLEWVSSISGSGRDTLYAESVKGRFTISRDNAKTTLYLQMNSLRPEDTAVYY |
| | | CTIGGSLSVSSQGTLVTVSS** |
| 325 | ACP406 | vprdcgckpcictvpevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnvqksnweagntftcsvlheglhnhhtekslshspgksggpGPAGLY |
| | | AQpgsaptssstkktqlqlehllldlqmilnginnyknpkltrmltfkfympkkatelkhlqcleeelkpleevlnlaqsknf |
| | | hlrprdlisninvivlelkgsettfmceyadetativeflnrwitfcqsiistltggggsggggsggggsggggsggggsgggg |
| | | ssggpGPAGLYAQpgsEVQLVESGGGLVQPGGSLRLSCAASGFTFSSYTLAWVRQA |
| | | PGKGLEWVAAIDSSSYTYSPDTVRGRFTISRDNAKNSLYLQMNSLRAEDTAVYY |
| | | CARDSNWDALDYWGQGTTVTVSSastkgpsvfplapsskstsggtaalgclvkdyfpepvtvswnsgal |
| | | tsgvhtfpavlqssglyslssvvtvpssslgtqtyicnvnhkpsntkvdkrvepksc** |
| 326 | ACP407 | vprdcgckpcictvpevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnvqksnweagntftcsvlheglhnhhtekslshspgksggpGPAGLY |
| | | AQpgsaptssstkktqlqlehllldlqmilnginnyknpkltrmltfkfympkkatelkhlqcleeelkpleevlnlaqsknf |
| | | hlrprdlisninvivlelkgsettfmceyadetativeflnrwitfcqsiistltggggsggggsggggsggggsggggsgggg |
| | | ssggpGPAGLYAQpgsEVQLVESGGGLVQPGGSLRLSCAASGFTFSSYTLAWVRQA |
| | | PGKcLEWVAAIDSSSYTYSPDTVRGRFTISRDNAKNSLYLQMNSLRAEDTAVYY |
| | | CARDSNWDALDYWGQGTTVTVSSGGGGSGGGGSGGGGSDIQMTQSPSSLSASV |
| | | GDRVTITCKAREKLWSAVAWYQQKPGKAPKALIYSASFRYSGVPSRFSGSGSGT |
| | | DFTLTISSLQPEDFATYYCQQYYTYPYTFGcGTKVEIK** |
| 327 | ACP408 | vprdcgckpcictvpevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnvqksnweagntftcsvlheglhnhhtekslshspgksggpGPAGLY |
| | | AQpgsaptssstkktqlqlehllldlqmilnginnyknpkltrmltfkfympkkatelkhlqcleeelkpleevlnlaqsknf |
| | | hlrprdlisninvivlelkgsettfmceyadetativeflnrwitfcqsiistltggggsggggsggggsggggsggggsgggg |
| | | ssggpGPAGLYAQpgsEVQLVESGGGLVQPGGSLRLSCAASGFTFSSYTLAWVRQA |
| | | PGKcLEWVAAIDSSSYTYSPDTVRGRFTISRDNAKNSLYLQMNSLRAEDTAVYY |
| | | CARDSNWDALDYWGQGTTVTVSSGGGGSGGGGSGGGGSDIQMTQSPSSLSASV |
| | | GDRVTITCKAREKLWSAVAWYQQKPGKAPKsLIYSASFRYSGVPSRFSGSGSGT |
| | | DFTLTISSLQPEDFATYYCQQYYTYPYTFGcGTKVEIK** |
| 328 | ACP409 | vprdcgckpcictvpevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnyqksnweagntftcsvlheglhnhhtekslshspgksggpGPAGLY |
| | | AQpgsaptssstkktqlqlehllldlqmilnginnyknpkltrmltfkfympkkatelkhlqcleeelkpleevlnlaqsknf |
| | | hlrprdlisninvivlelkgsettfmceyadetativeflnrwitfcqsiistltggggsggggsggggsggggsggggsgggg |
| | | ssggpGPAGLYAQpgsEVQLVESGGGLVQPGGSLRLSCAASGFTFSSYTLAWVRQA |
| | | PGKGLEWVAAIDSSSYTYSPDTVRGRFTISRDNAKNSLYLQMNSLRAEDTAVYY |
| | | CARDSNWDALDYWGcGTTVTVSSGGGGSGGGGSGGGGSDIQMTQSPSSLSASV |
| | | GDRVTITCKAREKLWSAVAWYQQKPGKcPKALIYSASFRYSGVPSRFSGSGSGT |
| | | DFTLTISSLQPEDFATYYCQQYYTYPYTFGGGTKVEIK** |
| 329 | ACP410 | vprdcgckpcictvpevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnvqksnweagntftcsvlheglhnhhtekslshspgksggpGPAGLY |
| | | AQpgsaptssstkktqlqlehllldlqmilnginnyknpkltrmltfkfympkkatelkhlqcleeelkpleevlnlaqsknf |
| | | hlrprdlisninvivlelkgsettfmceyadetativeflnrwitfcqsiistltggggsggggsggggsggggsggggsgggg |
| | | ssggpGPAGLYAQpgsEVQLVESGGGLVQPGGSLRLSCAASGFTFSSYTLAWVRQA |
| | | PGKcLEWVAAIDSSSYTYSPDTVRGRFTISRDNAKNSLYLQMNSLRAEDTAVYY |
| | | CARDSNWDALDYWGQGTTVTVSSGGGGSGGGGSGGGGSDIQMTQSPSSLSASV |
| | | GDRVTITCKVTEKVWGNVAWYQQKPGKAPISLIYSPSLRKSGVPSRFSGSGSGT |
| | | DFTLTISSLQPEDFATYYCQQYYTYPYTFGcGTKVEIK** |
| 330 | ACP411 | vprdcgckpcictvpevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnvqksnweagntftcsvlheglhnhhtekslshspgksggpGPAGLY |
| | | AQpgsaptssstkktqlqlehllldlqmilnginnyknpkltrmltfkfympkkatelkhlqcleeelkpleevlnlaqsknf |
| | | hlrprdlisninvivlelkgsettfmceyadetativeflnrwitfcqsiistltggggsggggsggggsggggsggggsgggg |
| | | ssggpGPAGLYAQpgsEVQLVESGGGLVQPGGSLRLSCAASGFTFSSYTLAWVRQA |
| | | PGKGLEWVAAIDSSSYTYSPDTVRGRFTISRDNAKNSLYLQMNSLRAEDTAVYY |
| | | CARDSNWDALDYWGcGTTVTVSSGGGGSGGGGSGGGGSDIQMTQSPSSLSASV |
| | | GDRVTITCKVTEKVWGNVAWYQQKPGKcPISLIYSPSLRKSGVPSRFSGSGSGTD |
| | | FTLTISSLQPEDFATYYCQQYYTYPYTFGGGTKVEIK** |
| 331 | ACP412 | vprdcgckpcictvpevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnyqksnweagntftcsvlheglhnhhtekslshspgksggpGPAGLY |
| | | AQpgsaptssstkktqlqlehllldlqmilnginnyknpkltrmltfkfympkkatelkhlqcleeelkpleevlnlaqsknf |
| | | hlrprdlisninvivlelkgsettfmceyadetativeflnrwitfcqsiistltggggsggggsggggsggggsggggsgggg |
| | | ssggpGPAGLYAQpgsevqllesggglvqpggslrlscaasgsifsanamgwyrqapgkqrelvavissggstnya |
| | | dsvkgrftisrdnskntvylqmnslraedtavyycmysgsyyytpndywgqgtlvtvss** |
| 332 | ACP413 | elcdddppeiphatfkamaykegtmlnceckrgfrriksgslymlctgnsshsswdnqcqctssatrnttkqvtpqpeeqk |
| | | erkttemqspmqpvdqaslpghcrepppweneateriyhfvvgqmvyyqcvqgyralhrgpaesvckmthgktrwtq |
| | | pqlictgemetsqfpgeekpqaspegrpesetsSlvtttdfqiqtemaatmetsiftteyqggggsggggsggggsggggs |
| | | ggggsggggsDIQMTQSPSSLSASVGDRVTITCKAREKLWSAVAWYQQKPGKAPKsL |
| | | IYSASFRYSGVPSRFSGSGSGTDFTLTISSLQPEDFATYYCQQYYTYPYTFGGGTK |
| | | VEIKrtvaapsyfifppsdeqlksgtasvvcllnnfypreakvqwkydnalqsgnsqesvteqdskdstyslsstltlskad |
| | | yekhkvyacevthqglsspvtksfnrgec** |
| 333 | ACP414 | elcdddppeiphatfkamaykegtmlnceckrgfrriksgslymlctgnsshsswdnqcqctssatmttkqvtpqpeeqk |
| | | erkttemqspmqpvdqaslpghcrepppweneateriyhfvvgqmvyyqcvqgyralhrgpaesyckmthgktrwtq |
| | | pqlictgemetsqfpgeekpqaspegrpesetsSlvtttdfqiqtemaatmetsiftteyqggggsggggsggggsggggs |
| | | ggggsggggsDIQMTQSPSSLSASVGDRVTITCKVTEKVWGNVAWYQQKPGKAPISL |
| | | IYSPSLRKSGVPSRFSGSGSGTDFTLTISSLQPEDFATYYCQQYYTYPYTFGGGTK |
| | | VEIKrtvaapsyfifppsdeqlksgtasvvcllnnfypreakvqwkydnalqsgnsqesvteqdskdstyslsstltlskad |
| | | yekhkvyacevthqglsspvtksfnrgec** |
| 334 | ACP415 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggsEVQLVESGGGLVQPGGSLRLSCAASGFTFSSYTLAWVRQAPGKcL |
| | | EWVAAIDSSSYTYSPDTVRGRFTISRDNAKNSLYLQMNSLRAEDTAVYYCARDS |
| | | NWDALDYWGQGTTVTVSSGGGGSGGGGSGGGGSDIQMTQSPSSLSASVGDRVT |
| | | ITCKAREKLWSAVAWYQQKPGKAPKsLIYSASFRYSGVPSRFSGSGSGTDFTLTIS |
| | | SLQPEDFATYYCQQYYTYPYTFGcGTKVEIKggggsggggsggggsggggsggggsggggsgg |
| | | ggsggggsggggselcdddppeiphatfkamaykegtmlnceckrgfrriksgslymlctgnsshsswdnqcqctssatr |
| | | nttkqvtpqpeeqkerkttemqspmqpvdqaslpghcrepppweneateriyhfvvgqmvyyqcvqgyralhrgpae |
| | | svckmthgktrwtqpqlictgemetsqfpgeekpqaspegrpesetsSlvtttdfqiqtemaatmetsiftteyqsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSS** |
| 335 | ACP416 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggsEVQLVESGGGLVQPGGSLRLSCAASGFTFSSYTLAWVRQAPGKG |
| | | LEWVAAIDSSSYTYSPDTVRGRFTISRDNAKNSLYLQMNSLRAEDTAVYYCARD |
| | | SNWDALDYWGcGTTVTVSSGGGGSGGGGSGGGGSDIQMTQSPSSLSASVGDRV |
| | | TITCKAREKLWSAVAWYQQKPGKcPKALIYSASFRYSGVPSRFSGSGSGTDFTLT |
| | | ISSLQPEDFATYYCQQYYTYPYTFGGGTKVEIKggggsggggsggggsggggsggggsggggs |
| | | ggggsggggsggggselcdddppeiphatfkamaykegtmlnceckrgfrriksgslymlctgnsshsswdnqcqctss |
| | | atrnttkqvtpqpeeqkerkttemqspmqpvdqaslpghcrepppweneateriyhfvvgqmvyyqcvqgyralhrgp |
| | | aesvckmthgktrwtqpqlictgemetsqfpgeekpqaspegrpesetsSlvtttdfqiqtemaatmetsiftteyqsggpG |
| | | PAGLYAQpgsEVQLVESGGGLVQPGNSLRLSCAASGFTFSKFGMSWVRQAPGKG |
| | | LEWVSSISGSGRDTLYAESVKGRFTISRDNAKTTLYLQMNSLRPEDTAVYYCTIG |
| | | GSLSVSSQGTLVTVSS** |
| 336 | ACP417 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggsEVQLVESGGGLVQPGGSLRLSCAASGFTFSSYTLAWVRQAPGKcL |
| | | EWVAAIDSSSYTYSPDTVRGRFTISRDNAKNSLYLQMNSLRAEDTAVYYCARDS |
| | | NWDALDYWGQGTTVTVSSGGGGSGGGGSGGGGSDIQMTQSPSSLSASVGDRVT |
| | | ITCKVTEKVWGNVAWYQQKPGKAPISLIYSPSLRKSGVPSRFSGSGSGTDFTLTIS |
| | | SLQPEDFATYYCQQYYTYPYTFGcGTKVEIKggggsggggsggggsggggsggggsggggsgg |
| | | ggsggggsggggselcdddppeiphatfkamaykegtmlnceckrgfrriksgslymlctgnsshsswdnqcqctssatr |
| | | nttkqvtpqpeeqkerkttemqspmqpvdqaslpghcrepppweneateriyhfvvgqmvyyqcvqgyralhrgpae |
| | | svckmthgktrwtqpqlictgemetsqfpgeekpqaspegrpesetsSlvtttdfqiqtemaatmetsiftteyqsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSS** |
| 337 | ACP418 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggsEVQLVESGGGLVQPGGSLRLSCAASGFTFSSYTLAWVRQAPGKG |
| | | LEWVAAIDSSSYTYSPDTVRGRFTISRDNAKNSLYLQMNSLRAEDTAVYYCARD |
| | | SNWDALDYWGcGTTVTVSSGGGGSGGGGSGGGGSDIQMTQSPSSLSASVGDRV |
| | | TITCKVTEKVWGNVAWYQQKPGKcPISLIYSPSLRKSGVPSRFSGSGSGTDFTLTI |
| | | SSLQPEDFATYYCQQYYTYPYTFGGGTKVEIKggggsggggsggggsggggsggggsggggsg |
| | | gggsggggsggggselcdddppeiphatfkamaykegtmlnceckrgfrriksgslymlctgnsshsswdnqcqctssat |
| | | rnttkqvtpqpeeqkerkttemqspmqpvdqaslpghcrepppweneateriyhfvvgqmvyyqcvqgyralhrgpae |
| | | svckmthgktrwtqpqlictgemetsqfpgeekpqaspegrpesetsSlvtttdfqiqtemaatmetsiftteyqsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSS** |
| 338 | ACP419 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggsevqllesggglvqpggslrlscaasgsifsanamgwyrqapgkqrelvavissggstnyadsvkgrft |
| | | isrdnskntvylqmnslraedtavyycmysgsyyytpndywgqgtlvtvssggggsggggsggggsggggsggggsg |
| | | gggsggggsggggsggggselcdddppeiphatfkamaykegtmlnceckrgfrriksgslymlctgnsshsswdnqc |
| | | qctssatrnttkqvtpqpeeqkerkttemqspmqpvdqaslpghcrepppweneateriyhfvvgqmvyyqcvqgyral |
| | | hrgpaesvckmthgktrwtqpqlictgemetsqfpgeekpqaspegrpesetsSlvtttdfqiqtemaatmetsiftteyqs |
| | | ggpGPAGLYAQpgsEVQLVESGGGLVQPGNSLRLSCAASGFTFSKFGMSWVRQAP |
| | | GKGLEWVSSISGSGRDTLYAESVKGRFTISRDNAKTTLYLQMNSLRPEDTAVYY |
| | | CTIGGSLSVSSQGTLVTVSS** |
| 339 | ACP420 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsggggsggggsggggsgg |
| | | ggsggggsggggsevqllesggglvqpggslrlscaasgsifsanamgwyrqapgkglelvavissggstnyadsvkgrft |
| | | isrdnskntvylqmnslraedtavyycmysgsyyytpndywgqgtlvtvssggggsggggsggggsggggsggggsg |
| | | gggsggggsggggsggggselcdddppeiphatfkamaykegtmlnceckrgfrriksgslymlctgnsshsswdnqc |
| | | qctssatrnttkqvtpqpeeqkerkttemqspmqpvdqaslpghcrepppweneateriyhfvvgqmvyyqcvqgyral |
| | | hrgpaesvckmthgktrwtqpqlictgemetsqfpgeekpqaspegrpesetsSlvtttdfqiqtemaatmetsiftteyqs |
| | | ggpGPAGLYAQpgsEVQLVESGGGLVQPGNSLRLSCAASGFTFSKFGMSWVRQAP |
| | | GKGLEWVSSISGSGRDTLYAESVKGRFTISRDNAKTTLYLQMNSLRPEDTAVYY |
| | | CTIGGSLSVSSQGTLVTVSS** |
| 340 | ACP421 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpGPAGLYAQpgsEVQLVESGGGLVQPGGSLRLSCAASGFTFSSYTLA |
| | | WVRQAPGKcLEWVAAIDSSSYTYSPDTVRGRFTISRDNAKNSLYLQMNSLRAED |
| | | TAVYYCARDSNWDALDYWGQGTTVTVSSGGGGSGGGGSGGGGSDIQMTQSPS |
| | | SLSASVGDRVTITCKAREKLWSAVAWYQQKPGKAPKsLIYSASFRYSGVPSRFSG |
| | | SGSGTDFTLTISSLQPEDFATYYCQQYYTYPYTFGcGTKVEIKggggsggggsggggsgg |
| | | ggsggggsggggsggggsggggsggggselcdddppeiphatfkamaykegtmlnceckrgfrriksgslymlctgnss |
| | | hsswdnqcqctssatrnttkqvtpqpeeqkerkttemqspmqpvdqaslpghcrepppweneateriyhfvvgqmvyy |
| | | qcvqgyralhrgpaesvckmthgktrwtqpqlictgemetsqfpgeekpqaspegrpesetsSlvtttdfqiqtemaatme |
| | | tsiftteyqggggsggggsggggsggggsggggsggggssggpGPAGLYAQpgsaptssstkktqlqlehllldlqm |
| | | ilnginnyknpkltrmltfkfympkkatelkhlqcleeelkpleevlnlaqsknfhlrprdlisninvivlelkgsettfmceya |
| | | detativeflnrwitfcqsiistlt** |
| 341 | ACP422 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpGPAGLYAQpgsEVQLVESGGGLVQPGGSLRLSCAASGFTFSSYTLA |
| | | WVRQAPGKGLEWVAAIDSSSYTYSPDTVRGRFTISRDNAKNSLYLQMNSLRAE |
| | | DTAVYYCARDSNWDALDYWGcGTTVTVSSGGGGSGGGGSGGGGSDIQMTQSP |
| | | SSLSASVGDRVTITCKAREKLW SAVAWYQQKPGKcPKALIYSASFRYSGVPSRFS |
| | | GSGSGTDFTLTISSLQPEDFATYYCQQYYTYPYTFGGGTKVEIKggggsggggsggggs |
| | | ggggsggggsggggsggggsggggsggggselcdddppeiphatfkamaykegtmlnceckrgfrriksgslymlctg |
| | | nsshsswdnqcqctssatrnttkqvtpqpeeqkerkttemqspmqpvdqaslpghcrepppweneateriyhfvvgqm |
| | | vyyqcvqgyralhrgpaesvckmthgktrwtqpqlictgemetsqfpgeekpqaspegrpesetsSlvtttdfqiqtemaa |
| | | tmetsiftteyqggggsggggsggggsggggsggggsggggssggpGPAGLYAQpgsaptssstkktqlqlehllld |
| | | lqmilnginnyknpkltrmlafympkkatelkhlqcleeelkpleevlnlaqsknfhlrprdlisninvivlelkgsettfm |
| | | ceyadetativeflnrwitfcqsiistlt** |
| 342 | ACP423 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpGPAGLYAQpgsEVQLVESGGGLVQPGGSLRLSCAASGFTFSSYTLA |
| | | WVRQAPGKcLEWVAAIDSSSYTYSPDTVRGRFTISRDNAKNSLYLQMNSLRAED |
| | | TAVYYCARDSNWDALDYWGQGTTVTVSSGGGGSGGGGSGGGGSDIQMTQSPS |
| | | SLSASVGDRVTITCKVTEKVWGNVAWYQQKPGKAPISLIYSPSLRKSGVPSRFSG |
| | | SGSGTDFTLTISSLQPEDFATYYCQQYYTYPYTFGcGTKVEIKggggsggggsggggsgg |
| | | ggsggggsggggsggggsggggsggggselcdddppeiphatfkamaykegtmlnceckrgfrriksgslymlctgnss |
| | | hsswdnqcqctssatrnttkqvtpqpeeqkerkttemqspmqpvdqaslpghcrepppweneateriyhfvvgqmvyy |
| | | qcvqgyralhrgpaesvckmthgktrwtqpqlictgemetsqfpgeekpqaspegrpesetsSlvtttdfqiqtemaatme |
| | | tsiftteyqggggsggggsggggsggggsggggsggggssggpGPAGLYAQpgsaptssstkktqlqlehllldlqm |
| | | ilnginnyknpkltrmltfkfympkkatelkhlqcleeelkpleevlnlaqsknfhlrprdlisninvivlelkgsettfmceya |
| | | detativeflnrwitfcqsiistlt** |
| 343 | ACP424 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpGPAGLYAQpgsEVQLVESGGGLVQPGGSLRLSCAASGFTFSSYTLA |
| | | WVRQAPGKGLEWVAAIDSSSYTYSPDTVRGRFTISRDNAKNSLYLQMNSLRAE |
| | | DTAVYYCARDSNWDALDYWGcGTTVTVSSGGGGSGGGGSGGGGSDIQMTQSP |
| | | SSLSASVGDRVTITCKVTEKVWGNVAWYQQKPGKcPISLIYSPSLRKSGVPSRFS |
| | | GSGSGTDFTLTISSLQPEDFATYYCQQYYTYPYTFGGGTKVEIKggggsggggsggggs |
| | | ggggsggggsggggsggggsggggsggggselcdddppeiphatfkamaykegtmlnceckrgfrriksgslymlctg |
| | | nsshsswdnqcqctssatrnttkqvtpqpeeqkerkttemqspmqpvdqaslpghcrepppweneateriyhfvvgqm |
| | | vyyqcvqgyralhrgpaesvckmthgktrwtqpqlictgemetsqfpgeekpqaspegrpesetsSlvtttdfqiqtemaa |
| | | tmetsiftteyqggggsggggsggggsggggsggggsggggssggpGPAGLYAQpgsaptssstkktqlqlehllld |
| | | lqmilnginnyknpkltrmlafympkkatelkhlqcleeelkpleevlnlaqsknfhlrprdlisninvivlelkgsettfm |
| | | ceyadetativeflnrwitfcqsiistlt** |
| 344 | Acp425 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpGPAGLYAQpgsevqllesggglvqpggslrlscaasgsifsanamgwyrqapgkqrelvaviss |
| | | ggstnyadsvkgrftisrdnskntvylqmnslraedtavyycmysgsyyytpndywgqgtlvtvssggggsggggsggg |
| | | gsggggsggggsggggsggggsggggsggggselcdddppeiphatfkamaykegtmlnceckrgfrriksgslymlc |
| | | tgnsshsswdnqcqctssatrnttkqvtpqpeeqkerkttemqspmqpvdqaslpghcrepppweneateriyhfvvgq |
| | | mvyyqcvqgyralhrgpaesvckmthgktrwtqpqlictgemetsqfpgeekpqaspegrpesetsSlvtttdfqiqtem |
| | | aatmetsiftteyqggggsggggsggggsggggsggggsggggssggpGPAGLYAQpgsaptssstkktqlqlehll |
| | | ldlqmilnginnyknpkltrmltfkfympkkatelkhlqcleeelkpleevlnlaqsknfhlrprdlisninvivlelkgsettf |
| | | mceyadetativeflnrwitfcqsiistlt** |
| 345 | ACP426 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpGPAGLYAQpgsevqllesggglvqpggslrlscaasgsifsanamgwyrqapgkglelvaviss |
| | | ggstnyadsvkgrftisrdnskntvylqmnslraedtavyycmysgsyyytpndywgqgtlvtvssggggsggggsggg |
| | | gsggggsggggsggggsggggsggggsggggselcdddppeiphatfkamaykegtmlnceckrgfrriksgslymlc |
| | | tgnsshsswdnqcqctssatrnttkqvtpqpeeqkerkttemqspmqpvdqaslpghcrepppweneateriyhfvvgq |
| | | mvyyqcvqgyralhrgpaesvckmthgktrwtqpqlictgemetsqfpgeekpqaspegrpesetsSlvtttdfqiqtem |
| | | aatmetsiftteyqggggsggggsggggsggggsggggsggggssggpGPAGLYAQpgsaptssstkktqlqlehll |
| | | ldlqmilnginnyknpkltrmltfkfympkkatelkhlqcleeelkpleevlnlaqsknfhlrprdlisninvivlelkgsettf |
| | | mceyadetativeflnrwitfcqsiistlt** |
| 346 | ACP427 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKcLEWVAAIDSSSYTYSPDTVRGRFTISRDNAKNSLYL |
| | | QMNSLRAEDTAVYYCARDSNWDALDYWGQGTTVTVSSGGGGSGGGGSGGGG |
| | | SDIQMTQSPSSLSASVGDRVTITCKAREKLWSAVAWYQQKPGKAPKsLIYSASFR |
| | | YSGVPSRFSGSGSGTDFTLTISSLQPEDFATYYCQQYYTYPYTFGcGTKVEIKgggg |
| | | sggggsggggsggggsggggsggggsevqllesggglvqpggslrlscaasgsifsanamgwyrqapgkqrelvaviss |
| | | ggstnyadsvkgrftisrdnskntvylqmnslraedtavyycmysgsyyytpndywgqgtlvtvss** |
| 347 | ACP428 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGcGTTVTVSSGGGGSGGGGSGGGG |
| | | SDIQMTQSPSSLSASVGDRVTITCKAREKLWSAVAWYQQKPGKcPKALIYSASFR |
| | | YSGVPSRFSGSGSGTDFTLTISSLQPEDFATYYCQQYYTYPYTFGGGTKVEIKggg |
| | | gsggggsggggsggggsggggsggggsevqllesggglvqpggslrlscaasgsifsanamgwyrqapgkqrelvavis |
| | | sggstnyadsvkgrftisrdnskntvylqmnslraedtavyycmysgsyyytpndywgqgtlvtvss** |
| 348 | ACP429 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKcLEWVAAIDSSSYTYSPDTVRGRFTISRDNAKNSLYL |
| | | QMNSLRAEDTAVYYCARDSNWDALDYWGQGTTVTVSSGGGGSGGGGSGGGG |
| | | SDIQMTQSPSSLSASVGDRVTITCKVTEKVWGNVAWYQQKPGKAPISLIYSPSLR |
| | | KSGVPSRFSGSGSGTDFTLTISSLQPEDFATYYCQQYYTYPYTFGcGTKVEIKgggg |
| | | sggggsggggsggggsggggsggggsevqllesggglvqpggslrlscaasgsifsanamgwyrqapgkqrelvaviss |
| | | ggstnyadsvkgrftisrdnskntvylqmnslraedtavyycmysgsyyytpndywgqgtlvtvss** |
| 349 | ACP430 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGcGTTVTVSSGGGGSGGGGSGGGG |
| | | SDIQMTQSPSSLSASVGDRVTITCKVTEKVWGNVAWYQQKPGKcPISLIYSPSLR |
| | | KSGVPSRFSGSGSGTDFTLTISSLQPEDFATYYCQQYYTYPYTFGGGTKVEIKggg |
| | | gsggggsggggsggggsggggsggggsevqllesggglvqpggslrlscaasgsifsanamgwyrqapgkqrelvavis |
| | | sggstnyadsvkgrftisrdnskntvylqmnslraedtavyycmysgsyyytpndywgqgtlvtvss** |
| 350 | ACP431 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKcLEWVAAIDSSSYTYSPDTVRGRFTISRDNAKNSLYL |
| | | QMNSLRAEDTAVYYCARDSNWDALDYWGQGTTVTVSSGGGGSGGGGSGGGG |
| | | SDIQMTQSPSSLSASVGDRVTITCKAREKLWSAVAWYQQKPGKAPKsLIYSASFR |
| | | YSGVPSRFSGSGSGTDFTLTISSLQPEDFATYYCQQYYTYPYTFGcGTKVEIKgggg |
| | | sggggsggggsggggsggggsggggsevqllesggglvqpggslrlscaasgsifsanamgwyrqapgkglelvaviss |
| | | ggstnyadsvkgrftisrdnskntvylqmnslraedtavyycmysgsyyytpndywgqgtlvtvss** |
| 351 | ACP432 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGcGTTVTVSSGGGGSGGGGSGGGG |
| | | SDIQMTQSPSSLSASVGDRVTITCKAREKLWSAVAWYQQKPGKcPKALIYSASFR |
| | | YSGVPSRFSGSGSGTDFTLTISSLQPEDFATYYCQQYYTYPYTFGGGTKVEIKggg |
| | | gsggggsggggsggggsggggsggggsevqllesggglvqpggslrlscaasgsifsanamgwyrqapgkglelvavis |
| | | sggstnyadsvkgrftisrdnskntvylqmnslraedtavyycmysgsyyytpndywgqgtlvtvss** |
| 352 | ACP433 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKcLEWVAAIDSSSYTYSPDTVRGRFTISRDNAKNSLYL |
| | | QMNSLRAEDTAVYYCARDSNWDALDYWGQGTTVTVSSGGGGSGGGGSGGGG |
| | | SDIQMTQSPSSLSASVGDRVTITCKVTEKVWGNVAWYQQKPGKAPISLIYSPSLR |
| | | KSGVPSRFSGSGSGTDFTLTISSLQPEDFATYYCQQYYTYPYTFGcGTKVEIKgggg |
| | | sggggsggggsggggsggggsggggsevqllesggglvqpggslrlscaasgsifsanamgwyrqapgkglelvaviss |
| | | ggstnyadsvkgrftisrdnskntvylqmnslraedtavyycmysgsyyytpndywgqgtlvtvss** |
| 353 | ACP434 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGcGTTVTVSSGGGGSGGGGSGGGG |
| | | SDIQMTQSPSSLSASVGDRVTITCKVTEKVWGNVAWYQQKPGKcPISLIYSPSLR |
| | | KSGVPSRFSGSGSGTDFTLTISSLQPEDFATYYCQQYYTYPYTFGGGTKVEIKggg |
| | | gsggggsggggsggggsggggsggggsevqllesggglvqpggslrlscaasgsifsanamgwyrqapgkglelvavis |
| | | sggstnyadsvkgrftisrdnskntvylqmnslraedtavyycmysgsyyytpndywgqgtlvtvss** |
| 265 | ACP435 | DIQMTQSPSSLSASVGDRVTITCKAREKLWSAVAWYQQKPGKAPKsLIYSASFR |
| | | YSGVPSRFSGSGSGTDFTLTISSLQPEDFATYYCQQYYTYPYTFGGGTKVEIKrtvaa |
| | | psvfifppsdeqlksgtasvvcllnnfypreakvqwkvdnalqsgnsqesvteqdskdstyslsstltlskadyekhkvyac |
| | | evthqglsspvtksfnrgecggggsggggsggggsggggsggggsggggsevqllesggglvqpggslrlscaasgsifsa |
| | | namgwyrqapgkqrelvavissggstnyadsvkgrftisrdnskntvylqmnslraedtavyycmysgsyyytpndyw |
| | | gqgtlvtvss** |
| 355 | ACP371 | aptssstkktqlqlehllldlqmilnginnyknpkltrmltfldympkkatelkhlqcleeelkpleevlnlaqsknf |
| | | hlrprdlisninvivlelkgsettfmceyadetativeflnrwitfcqsiistltsggpGPAGLYAQpgsEVQ |
| | | LVESGGGLVQPGNSLRLSCAASGFTFSKFGMSWVRQAPGKGLEWVSSIS |
| | | GSGRDTLYAESVKGRFTISRDNAKTTLYLQMNSLRPEDTAVYYCTIGGSL |
| | | SVSSQGTLVTVSSggggsggggsggggsggggsggggsggggssggpGPAGLYAQpgsEV |
| | | QLVESGGGLVQPGGSLRLSCAASGFTFSSYTLAWVRQAPGKcLEWVAAI |
| | | DSSSYTYSPDTVRGRFTISRDNAKNSLYLQMNSLRAEDTAVYYCARDSN |
| | | WDALDYWGQGTTVTVSSGGGGSGGGGSGGGGSDIQMTQSPSSLSASVG |
| | | DRVTITCKASQNVGTNVGWYQQKPGKAPKALIYSASFRYSGVPSRFSGS |
| | | GSGTDFTLTISSLQPEDFATYYCQQYYTYPYTFGcGTKVEIK** |
| 356 | ACP372 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGcGTTVTVSSGGGGSGGGGSGGGG |
| | | SDIQMTQSPSSLSASVGDRVTITCKASQNVGTNVGWYQQKPGKcPKALIYSASFR |
| | | YSGVPSRFSGSGSGTDFTLTISSLQPEDFATYYCQQYYTYPYTFGGGTKVEIK** |
| 357 | ACP373 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKcLEWVAAIDSSSYTYSPDTVRGRFTISRDNAKNSLYL |
| | | QMNSLRAEDTAVYYCARDSNWDALDYWGQGTTVTVSSGGGGSGGGGSGGGG |
| | | SDIQMTQSPSSLSASVGDRVTITCKAREKLWSAVAWYQQKPGKAPKALIYSASF |
| | | RYSGVPSRFSGSGSGTDFTLTISSLQPEDFATYYCQQYYTYPYTFGcGTKVEIK** |
| 358 | ACP374 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGcGTTVTVSSGGGGSGGGGSGGGG |
| | | SDIQMTQSPSSLSASVGDRVTITCKAREKLWSAVAWYQQKPGKcPKALIYSASFR |
| | | YSGVPSRFSGSGSGTDFTLTISSLQPEDFATYYCQQYYTYPYTFGGGTKVEIK** |
| 359 | ACP375 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKcLEWVAAIDSSSYTYSPDTVRGRFTISRDNAKNSLYL |
| | | QMNSLRAEDTAVYYCARDSNWDALDYWGQGTTVTVSSGGGGSGGGGSGGGG |
| | | SDIQMTQSPSSLSASVGDRVTITCKVTEKVWGNVAWYQQKPGKAPISLIYSPSLR |
| | | KSGVPSRFSGSGSGTDFTLTISSLQPEDFATYYCQQYYTYPYTFGcGTKVEIK** |
| 360 | ACP376 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGcGTTVTVSSGGGGSGGGGSGGGG |
| | | SDIQMTQSPSSLSASVGDRVTITCKVTEKVWGNVAWYQQKPGKcPISLIYSPSLR |
| | | KSGVPSRFSGSGSGTDFTLTISSLQPEDFATYYCQQYYTYPYTFGGGTKVEIK** |
| 361 | ACP377 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsevqllesggglvqpggslrlscaasgsifsanamgwyrq |
| | | apgkqrelvavissggstnyadsvkgrftisrdnskntvylqmnslraedtavyycmysgsyyytpndywgqgtlvtvss |
| | | ** |
| 362 | ACP378 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGQGTTVTVSSastkgpsvfplapsskstsggt |
| | | aalgclvkdyfpepvtvswnsgaltsgvhtfpavlqssglyslssvvtvpssslgtqtyicnvnhkpsntkvdkrvepksc* |
| | | * |
| 363 | ACP379 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKGLEWVAAIDSSSYTYSPDTVRGRFTISRDNAKNSLYLQMNSLRAEDTAV |
| | | YYCARDSNWDALDYWGQGTTVTVSSastkgpsvfplapsskstsggtaalgclvkdyfpepvtvswn |
| | | sgaltsgvhtfpavlqssglyslssvvtvpssslgtqtyicnvnhkpsntkvdkrvepksc** |
| 364 | ACP368 | EVQLVESGGGLVQPGGSLRLSCAASGFTFSSYTLAWVRQAPGKGLEWVA |
| | | AIDSSSYTYSPDTVRGRFTISRDNAKNSLYLQMNSLRAEDTAVYYCARDS |
| | | NWDALDYWGQGTTVTVSSsggpGPAGLYAQpgsDIQMTQSPSSLSASVGD |
| | | RVTITCKVTEKVWGNVAWYQQKPGKAPISLIYSPSLRKSGVPSRFSGSGS |
| | | GTDFTLTISSLQPEDFATYYCQQYYTYPYTFGGGTKVEIKHHHHHH |
| 365 | ACP365 | EVQLVESGGGLVQPGGSLRLSCAASGFTFSSYTLAWVRQAPGKGLEWVA |
| | | AIDSSSYTYSPDTVRGRFTISRDNAKNSLYLQMNSLRAEDTAVYYCARDS |
| | | NWDALDYWGQGTTVTVSSsggpGPAGLYAQpgsDIQMTQSPSSLSASVGD |
| | | RVTITCKAREKLWSAVAWYQQKPGKAPKALIYSASFRYSGVPSRFSGSGS |
| | | GTDFTLTISSLQPEDFATYYCQQYYTYPYTFGGGTKVEIKHHHHHH |
| 366 | ACP366 | EVQLVESGGGLVQPGGSLRLSCAASGFTFSSYTLAWVRQAPGKGLEWVAAIDSS |
| | | SYTYSPDTVRGRFTISRDNAKNSLYLQMNSLRAEDTAVYYCARDSNWDALDYW |
| | | GQGTTVTVSSsggpGPAGLYAQpgsDIQMTQSPSSLSASVGDRVTITCKAREKLWS |
| | | AVAWYQQKPGKAPKsLIYSASFRYSGVPSRFSGSGSGTDFTLTISSLQPEDFATYY |
| | | CQQYYTYPYTFGGGTKVEIKHHHHHH |
| 367 | ACP284 | EVQLVESGGGLVQPGNSLRLSCAASGFTFSKFGMSWVRQAPGKGLEWVS |
| | | SISGSGRDTLYAESVKGRFTISRDNAKTTLYLQMNSLRPEDTAVYYCTIG |
| | | GSLSVSSQGTLVTVSSSGGPGPAGMKGLPGSrvipvsgparclsqsrnllkttddmvktar |
| | | eklkhysctaedidheditrdqtstlktclplelhknesclatretssttrgsclppqktslmmtlclgsiyedlkmyq |
| | | tefqainaalqnhnhqqiildkgmlvaidelmqslnhngetlrqkppvgeadpyrvkmklcillhafstrvvtinr |
| | | vmgylssaSGGPGPAGMKGLPGSggggsggggsggggsggggsggggsggggsQSVLTQP |
| | | PSVSGAPGQRVTISCSGSRSNIGSNTVKWYQQLPGTAPKLLIYYNDQRPSG |
| | | VPDRFSGSKSGTSASLAITGLQAEDEADYYCQSYDRYTHPALLFGTGTKV |
| | | TVLggggsggggsggggsQVQLVESGGGVVQPGRSLRLSCAASGFTFSSYGMH |
| | | WVRQAPGKGLEWVAFIRYDGSNKYYADSVKGRFTISRDNSKNTLYLQM |
| | | NSLRAEDTAVYYCKTHGSHDNWGQGTMVTVSS |
| 368 | ACP285 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSSGGPGPAGMKGLPGSiwelkkdvyvveldwypdapgemvvltcdtpeedgitwtldqssevlg |
| | | sgktltiqvkefgdagqytchkggevlshsllllhkkedgiwstdilkdqkepknktflrceaknysgrftcwwlttistdltfs |
| | | vkssrgssdpqgvtcgaatlsaervrgdnkeyeysvecqedsaSpaaeeslpievmvdavhklkyenytssffirdiikpd |
| | | ppknlqlkplknsrqvevsweypdtwstphsyfsltfcvqvqgkskrekkdrvftdktsatvicrknasisvraqdryysss |
| | | wsewasvpcsggggsggggsggggsrvipvsgparclsqsrnllkttddmvktareklkhysctaedidheditrdqtstlk |
| | | tclplelhknesSlatretssttrgsclppqktslmmtlclgsiyedlkmyqtefqainaalqnhnhqqiildkgmlvaidelm |
| | | qslnhngetlrqkppygeadpyrvkmklcillhafstrvvtinrvmgylssaSGGPGPAGMKGLPGSggggsg |
| | | gggsggggsggggsggggsggggsQSVLTQPPSVSGAPGQRVTISCSGSRSNIGSNTVKWY |
| | | QQLPGTAPKLLIYYNDQRPSGVPDRFSGSKSGTSASLAITGLQAEDEADYYCQSY |
| | | DRYTHPALLFGTGTKVTVLggggsggggsggggsQVQLVESGGGVVQPGRSLRLSCAA |
| | | SGFTFSSYGMHWVRQAPGKGLEWVAFIRYDGSNKYYADSVKGRFTISRDNSKN |
| | | TLYLQMNSLRAEDTAVYYCKTHGSHDNWGQGTMVTVSS |
| 369 | ACP286 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSSGGPGPAGMKGLPGSiwelkkdvyvveldwypdapgemvvltcdtpeedgitwtldqssevlg |
| | | sgktltiqvkefgdagqytchkggevlshsllllhkkedgiwstdilkdqkepknktflrceaknysgrftcwwlttistdltfs |
| | | vkssrgssdpqgvtcgaatlsaervrgdnkeyeysvecqedsacpaaeeslpievmvdavhklkyenytssffirdiikpd |
| | | ppknlqlkplknsrqvevsweypdtwstphsyfsltfcvqvqgkskrekkdrvftdktsatvicrknasisvraqdryysss |
| | | wsewasvpcsggggsggggsggggsggggsrvipvsgparclsqsrnllkaddmvktareklkhysctaedidheditr |
| | | dqtstlktclplelhknesclatretssttrgsclppqktslmmtlclgsiyedlkmyqtefqainaalqnhnhqqiildkgmlv |
| | | aidelmqslnhngetlrqkppvgeadpyrvkmklcillhafstrvvtinrvmgylssaSGGPGPAGMKGLPGSg |
| | | gggsggggsggggsggggsggggsggggsQSVLTQPPSVSGAPGQRVTISCSGSRSNIGSNTV |
| | | KWYQQLPGTAPKLLIYYNDQRPSGVPDRFSGSKSGTSASLAITGLQAEDEADYY |
| | | CQSYDRYTHPALLFGTGTKVTVLggggsggggsggggsQVQLVESGGGVVQPGRSLRL |
| | | SCAASGFTFSSYGMHWVRQAPGKGLEWVAFIRYDGSNKYYADSVKGRFTISRD |
| | | NSKNTLYLQMNSLRAEDTAVYYCKTHGSHDNWGQGTMVTVSS |
| 370 | ACP287 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSSGGPGPAGMKGLPGSiwelkkdvyvveldwypdapgemvvltcdtpeedgitwtldqssevlg |
| | | sgktltiqvkefgdagqytchkggevlshsllllhkkedgiwstdilkdqkepknktflrceaknysgrftcwwlttistdltfs |
| | | vkssrgssdpqgvtcgaatlsaervrgdnkeyeysvecqedsacpaaeeslpievmvdavhklkyenytssffirdiikpd |
| | | ppknlqlkplknsrqvevsweypdtwstphsyfsltfcvqvqgkskrekkdrvftdktsatvicrknasisvraqdryysss |
| | | wsewasvpcsggggsggggsggggsrvipvsgparclsqsrnllkttddmvktareklkhysctaedidheditrdqtstlk |
| | | tclplelhknesclatretssttrgsclppqktslmmtlclgsiyedlkmyqtefqainaalqnhnhqqiildkgmlvaidelm |
| | | qslnhngetlrqkppvgeadpyrvkmklcillhafstrvvtinrvmgylssaSGGPGPAGMKGLPGSggggsg |
| | | gggsggggsggggsggggsggggsQSVLTQPPSVSGAPGQRVTISCSGSRSNIGSNTVKWY |
| | | QQLPGTAPKLLIYYNDQRPSGVPDRFSGSKSGTSASLAITGLQAEDEADYYCQSY |
| | | DRYTHPALLFGcGTKVTVLggggsggggsggggsQVQLVESGGGVVQPGRSLRLSCAA |
| | | SGFTFSSYGMHWVRQAPGKcLEWVAFIRYDGSNKYYADSVKGRFTISRDNSKNT |
| | | LYLQMNSLRAEDTAVYYCKTHGSHDNWGQGTMVTVSS |
| 371 | ACP288 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSSGGPGPAGMKGLPGSiwelkkdvyvveldwypdapgemvvltcdtpeedgitwtldqssevlg |
| | | sgktltiqvkefgdagqytchkggevlshsllllhkkedgiwstdilkdqkepknktflrceaknysgrftcwwlttistdltfs |
| | | vkssrgssdpqgvtcgaatlsaervrgdnkeyeysvecqedsacpaaeeslpievmvdavhklkyenytssffirdiikpd |
| | | ppknlqlkplknsrqveysweypdtwstphsyfsltfcvqvqgkskrekkdrvftdktsatvicrknasisvraqdryysss |
| | | wsewasvpcsggggsggggsggggsrvipvsgparclsqsrnllkttddmvktareklkhysctaedidheditrdqtstlk |
| | | tclplelhknesclatretssttrgsclppqktslmmtlclgsiyedlkmyqtefqainaalqnhnhqqiildkgmlvaidelm |
| | | qslnhngetlrqkppvgeadpyrvkmklcillhafstrvvtinrvmgylssaSGGPGPAGMKGLPGSggggsg |
| | | gggsggggsggggsggggsggggsQSVLTQPPSVSGAPGQRVTISCSGSRSNIGSNTVKWY |
| | | QQLPGTcPKLLIYYNDQRPSGVPDRFSGSKSGTSASLAITGLQAEDEADYYCQSY |
| | | DRYTHPALLFGTGTKVTVLggggsggggsggggsQVQLVESGGGVVQPGRSLRLSCAA |
| | | SGFTFSSYGMHWVRQAPGKGLEWVAFIRYDGSNKYYADSVKGRFTISRDNSKN |
| | | TLYLQMNSLRAEDTAVYYCKTHGSHDNWGcGTMVTVSS |
| 372 | ACP289 | aptssstkktqlqlehllldlqmilnginnyknpkltrmltfldympkkatelkhlqcleeelkpleevlnlaqsknf |
| | | hlrprdlisninvivlelkgsettfmceyadetativeflnrwitfcqsiistltsggpgpagmkglpgsevqlvesg |
| | | gglvqpgnslrlscaasgftfskfgmswvrqapgkglewvssisgsgrdtlyaesvkgrftisrdnakttlylqmn |
| | | slrpedtavyyctiggslsvssqgtlvtvssggggsggggsggggsggggsggggsggggssggpgpagmkgl |
| | | pgsevqlvesggglvqpggslrlscaasgftfssytlawvrqapgkglewvaaidsssvtvspdtvrgrftisrdna |
| | | knslylqmnslraedtavyycardsnwdaldywgqgttvtvssggggsggggsggggsdiqmtqspsslsas |
| | | vgdrvtitckasqnvgtnvgwyqqkpgkapkaliysasfrysgvpsrfsgsgsgtdftltisslqpedfatyycqq |
| | | yytypytfgggtkveikhhhhhh |
| 373 | ACP290 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpgpagmkglpgsevqlvesggglvqpgnslrlsca |
| | | asgftfskfgmswvrqapgkglewvssisgsgrdtlyaesvkgrftisrdnakttlylqmnslrpedtavyyctiggslsvss |
| | | qgtlvtvssggggsggggsggggsggggsggggsggggssggpgpagmkglpgQVQLQESGGGLVQTGG |
| | | SLRLSCTTSGTIFSGYTMGWYRQAPGEQRELVAVISGGGDTNYADSVKGRFTISR |
| | | DNTKDTMYLQMNSLKPEDTAVYYCYSREVTPPWKLYWGQGTQVTVSSAAAYP |
| | | YDVPDYGSHHHHHH |
| 374 | ACP291 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpgpagmkglpgsevqlvesggglvqpgnslrlsca |
| | | asgftfskfgmswvrqapgkglewvssisgsgrdtlyaesvkgrftisrdnakttlylqmnslrpedtavyyctiggslsvss |
| | | qgtlvtvssggggsggggsggggsggggsggggsggggssggpgpagmkglpgQVQLQESGGGLVQEGG |
| | | SLRLSCAASERIFSTDVMGWYRQAAEKQRELVAVVSARGTTNYLDAVKGRFTIS |
| | | RDNARNTLTLQMNDLKPEDTASYYCYVRETTSPWRIYWGQGTQVTVSSAAAYP |
| | | YDVPDYGSHHHHHH |
| 375 | ACP292 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpgpagmkglpgsevqlvesggglvqpgnslrlsca |
| | | asgftfskfgmswvrqapgkglewvssisgsgrdtlyaesvkgrftisrdnakttlylqmnslrpedtavyyctiggslsyss |
| | | qgtlvtvssggggsggggsggggsggggsggggsggggssggpgpagmkglpgQVQLQESGGGLVQAG |
| | | GSLRLSCAASGSIFSANAMGWYRQAPGKQRELVAVISSGGSTNYADSVKGRFTI |
| | | SRDNAKNTVYLQMNSLKPEDTAVYYCMYSGSYYYTPNDYWGQGTQVTVSSAA |
| | | AYPYDVPDYGSHHHHHH |
| 376 | ACP296 | aptssstkktqlqlehllldlqmilnginnyknpkltrmltfldympkkatelkhlqcleeelkpleevlnlaqsknf |
| | | hlrprdlisninvivlelkgsettfmceyadetativeflnrwitfcqsiistltSGGPGPAGMKGLPGSE |
| | | VQLVESGGGLVQPGNSLRLSCAASGFTFSKFGMSWVRQAPGKGLEWVSS |
| | | ISGSGRDTLYAESVKGRFTISRDNAKTTLYLQMNSLRPEDTAVYYCTIGG |
| | | SLSVSSQGTLVTVSSggggsggggsggggsggggsggggsggggsSGGPGPAGMKGLP |
| | | GSEVQLVESGGGLVQPGGSLRLSCAASGFTFSSYTLAWVRQAPGKGLEW |
| | | VAAIDSSSYTYSPDTVRGRFTISRDNAKNSLYLQMNSLRAEDTAVYYCAR |
| | | DSNWDALDYWGQGTTVTVSSSGGPGPAGMKGLPGSDIQMTQSPSSLSAS |
| | | VGDRVTITCKASQNVGTNVGWYQQKPGKAPKALIYSASFRYSGVPSRFS |
| | | GSGSGTDFTLTISSLQPEDFATYYCQQYYTYPYTFGGGTKVEIKEIREIRREI |
| | | EPEA** |
| 377 | Acp297 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltSGGPGPAGMKGLPGSEVQLVESGGG |
| | | LVQPGNSLRLSCAASGFTFSKFGMSWVRQAPGKGLEWVSSISGSGRDTLYAESV |
| | | KGRFTISRDNAKTTLYLQMNSLRPEDTAVYYCTIGGSLSVSSQGTLVTVSSggggsg |
| | | gggsggggsggggsggggsggggsSGGPGPAGMKGLPGSEVQLVESGGGLVQPGGSLRLS |
| | | CAASGFTFSSYTLAWVRQAPGKGLEWVAAIDSSSYTYSPDTVRGRFTISRDNAK |
| | | NSLYLQMNSLRAEDTAVYYCARDSNWDALDYWGQGTTVTVSSGGGGSGGGGS |
| | | GGGGSDIQMTQSPSSLSASVGDRVTITCKASQNVGTNVGWYQQKPGKAPKLLIY |
| | | SASFRYSGVPSRFSGSGSGTDFTLTISSLQPEDFATYYCQQYYTYPYTFGGGTKV |
| | | EIKHHHHHHEPEA** |
| 378 | ACP298 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltSGGPGPAGMKGLPGSEVQLVESGGG |
| | | LVQPGNSLRLSCAASGFTFSKFGMSWVRQAPGKGLEWVSSISGSGRDTLYAESV |
| | | KGRFTISRDNAKTTLYLQMNSLRPEDTAVYYCTIGGSLSVSSQGTLVTVSSggggsg |
| | | gggsggggsggggsggggsggggsSGGPGPAGMKGLPGSEVQLVESGGGLVQPGGSLRLS |
| | | CAASGFTFSSYTLAWVRQAPGKGLEWVAAIDSSSYTYSPDTVRGRFTISRDNAK |
| | | NSLYLQMNSLRAEDTAVYYCARDSNWDALDYWGQGTTVTVSSGGGGSGGGGS |
| | | GGGGSDIQMTQSPSSLSASVGDRVTITCKASQNVGTNVGWYQQKPGKAPKGLIY |
| | | SASFRYSGVPSRFSGSGSGTDFTLTISSLQPEDFATYYCQQYYTYPYTFGGGTKV |
| | | EIKHHHHHHEPEA** |
| 379 | ACP299 | aptssstkktqlqlehllldlqmilnginnyknpkltrmltfidympkkatelkhlqcleeelkpleevlnlaqsknfhlrprdli |
| | | sninvivlelkgsettfmceyadetativeflnrwitfSqsiistltSGGPGPAGMKGLPGSEVQLVESGGG |
| | | LVQPGNSLRLSCAASGFTFSKFGMSWVRQAPGKGLEWVSSISGSGRDTLYAESV |
| | | KGRFTISRDNAKTTLYLQMNSLRPEDTAVYYCTIGGSLSVSSQGTLVTVSSggggsg |
| | | gggsggggsggggsggggsggggsSGGPGPAGMKGLPGSEVQLVESGGGLVQPGGSLRLS |
| | | CAASGFTFSSYTLAWVRQAPGKGLEWVAAIDSSSYTYSPDTVRGRFTISRDNAK |
| | | NSLYLQMNSLRAEDTAVYYCARDSNWDALDYWGQGTTVTVSSGGGGSGGGGS |
| | | GGGGSDIQMTQSPSSLSASVGDRVTITCKASQNVGTNVGWYQQKPGKAPKALIY |
| | | SASFRYSGVPSRFSGSGSGTDFTLTISSLQPEDFATYYCQQYYTYPYTFGGGTKV |
| | | EIKHHHHHHEPEA** |
| 380 | ACP300 | aptssstkktqlqlehllldlqmilnginnyknpkltrmltfidympkkatelkhlqcleeelkpleevlnlaqsknf |
| | | hlrprdlisninvivlelkgsettfmceyadetativeflnrwitfcqsiistltSGGPGPAGMKGLPGSda |
| | | hksevahrfkdlgeenfkalvliafaqylqqcpfedhvklvnevtefaktcvadesaencdkslhtlfgdklctvat |
| | | lretygemadccakqepernecflqhkddnpnlprlvrpevdvmctafhdneetflkkylyeiarrhpyfyape |
| | | llffakrykaafteccqaadkaacllpkldelrdegkassakqrlkcaslqkfgerafkawavarlsqrfpkaefae |
| | | vsklvtdltkvhtecchgdllecaddradlakyicenqdsissklkeccekpllekshciaevendempadlpsla |
| | | adfveskdvcknyaeakdvflgmflyeyarrhpdysvvlllrlaktyettlekccaaadphecyakvfdefkplv |
| | | eepqnlikqncelfeqlgeykfqnallvrytkkvpqvstptlvevsrnlgkvgskcckhpeakrmpcaedylsv |
| | | vlnqlcvlhektpvsdrvtkccteslvnrrpcfsalevdetyvpkefnaetftfhadictlsekerqikkqtalvelvk |
| | | hkpkatkeqlkavmddfaafvekcckaddketcfaeegkklvaasqaalglggggsggggsggggsggggs |
| | | ggggsggggsSGGPGPAGMKGLPGSEVQLVESGGGLVQPGGSLRLSCAASGF |
| | | TFSSYTLAWVRQAPGKGLEWVAAIDSSSYTYSPDTVRGRFTISRDNAKNS |
| | | LYLQMNSLRAEDTAVYYCARDSNWDALDYWGQGTTVTVSSGGGGSGG |
| | | GGSGGGGSDIQMTQSPSSLSASVGDRVTITCKASQNVGTNVGWYQQKPG |
| | | KAPKALIYSASFRYSGVPSRFSGSGSGTDFTLTISSLQPEDFATYYCQQYY |
| | | TYPYTFGGGTKVEIKHHHHHHEPEA** |
| 381 | ACP302 | aptssstkktqlqlehllldlqmilnginnyknpkltrmltfkfympkkatelkhlqcleeelkpleevlnlaqsknf |
| | | hlrprdlisninvivlelkgsettfmceyadetativeflnrwitfcqsiistltSGGPGPAGMKGLPGSE |
| | | AHKSEIAHRYNDLGEQHFKGLVLIAFSQYLQKCSYDEHAKLVQEVTDFA |
| | | KTCVADESAANCDKSLHTLFGDKLCAIPNLRENYGELADCCTKQEPERN |
| | | ECFLQHKDDNPSLPPFERPEAEAMCTSFKENPTTFMGHYLHEVARRHPYF |
| | | YAPELLYYAEQYNEILTQCCAEADKESCLTPKLDGVKEKALVSSVRQRM |
| | | KCSSMQKFGERAFKAWAVARLSQTFPNADFAEITKLATDLTKVNKECCH |
| | | GDLLECADDRAELAKYMCENQATISSKLQTCCDKPLLKKAHCLSEVEHD |
| | | TMPADLPAIAADFVEDQEVCKNYAEAKDVFLGTFLYEYSRRHPDYSVSL |
| | | LLRLAKKYEATLEKCCAEANPPACYGTVLAEFQPLVEEPKNLVKTNCDL |
| | | YEKLGEYGFQNAILVRYTQKAPQVSTPTLVEAARNLGRVGTKCCTLPED |
| | | QRLPCVEDYLSAILNRVCLLHEKTPVSEHVTKCCSGSLVERRPCFSALTV |
| | | DETYVPKEFKAETFTFHSDICTLPEKEKQIKKQTALAELVKHKPKATAEQ |
| | | LKTVMDDFAQFLDTCCKAADKDTCFSTEGPNLVTRCKDALAggggsggggsg |
| | | gggsggggsggggsggggsSGGPGPAGMKGLPGSEVQLVESGGGLVQPGGSLRL |
| | | SCAASGFTFSSYTLAWVRQAPGKGLEWVAAIDSSSYTYSPDTVRGRFTIS |
| | | RDNAKNSLYLQMNSLRAEDTAVYYCARDSNWDALDYWGQGTTVTVSS |
| | | GGGGSGGGGSGGGGSDIQMTQSPSSLSASVGDRVTITCKASQNVGTNVG |
| | | WYQQKPGKAPKALIYSASFRYSGVPSRFSGSGSGTDFTLTISSLQPEDFAT |
| | | YYCQQYYTYPYTFGGGTKVEIKHHHHHH** |
| 382 | ACP303 | EAHKSEIAHRYNDLGEQHFKGLVLIAFSQYLQKCSYDEHAKLVQEVTDFAKTCV |
| | | ADESAANCDKSLHTLFGDKLCAIPNLRENYGELADCCTKQEPERNECFLQHKDD |
| | | NPSLPPFERPEAEAMCTSFKENPTTFMGHYLHEVARRHPYFYAPELLYYAEQYN |
| | | EILTQCCAEADKESCLTPKLDGVKEKALVSSVRQRMKCSSMQKFGERAFKAWA |
| | | VARLSQTFPNADFAEITKLATDLTKVNKECCHGDLLECADDRAELAKYMCENQ |
| | | ATISSKLQTCCDKPLLKKAHCLSEVEHDTMPADLPAIAADFVEDQEVCKNYAEA |
| | | KDVFLGTFLYEYSRRHPDYSVSLLLRLAKKYEATLEKCCAEANPPACYGTVLAE |
| | | FQPLVEEPKNLVKTNCDLYEKLGEYGFQNAILVRYTQKAPQVSTPTLVEAARNL |
| | | GRVGTKCCTLPEDQRLPCVEDYLSAILNRVCLLHEKTPVSEHVTKCCSGSLVERR |
| | | PCFSALTVDETYVPKEFKAETFTFHSDICTLPEKEKQIKKQTALAELVKHKPKAT |
| | | AEQLKTVMDDFAQFLDTCCKAADKDTCFSTEGPNLVTRCKDALASGGPGPAGM |
| | | KGLPGStfkfympkkatelkhlqcleeelkpleevlnlaqsknfhlrprdlisninvivlelkgsettfmceyadetativef |
| | | lnrwitfcqsiistltGGssstkktqlqlehllldlqmilnginnyknpkltrmlSGGPGPAGMKGLPGSEAHK |
| | | SEIAHRYNDLGEQHFKGLVLIAFSQYLQKCSYDEHAKLVQEVTDFAKTCVADES |
| | | AANCDKSLHTLFGDKLCAIPNLRENYGELADCCTKQEPERNECFLQHKDDNPSL |
| | | PPFERPEAEAMCTSFKENPTTFMGHYLHEVARRHPYFYAPELLYYAEQYNEILTQ |
| | | CCAEADKESCLTPKLDGVKEKALVSSVRQRMKCSSMQKFGERAFKAWAVARL |
| | | SQTFPNADFAEITKLATDLTKVNKECCHGDLLECADDRAELAKYMCENQATISS |
| | | KLQTCCDKPLLKKAHCLSEVEHDTMPADLPAIAADFVEDQEVCKNYAEAKDVF |
| | | LGTFLYEYSRRHPDYSVSLLLRLAKKYEATLEKCCAEANPPACYGTVLAEFQPL |
| | | VEEPKNLVKTNCDLYEKLGEYGFQNAILVRYTQKAPQVSTPTLVEAARNLGRV |
| | | GTKCCTLPEDQRLPCVEDYLSAILNRVCLLHEKTPVSEHVTKCCSGSLVERRPCF |
| | | SALTVDETYVPKEFKAETFTFHSDICTLPEKEKQIKKQTALAELVKHKPKATAEQ |
| | | LKTVMDDFAQFLDTCCKAADKDTCFSTEGPNLVTRCKDALAHHHHHH** |
| 383 | ACP304 | aptssstkktqlqlehllldlqmilnginnyknpkltrmltfldympkkatelkhlqcleeelkpleevlnlaqsknf |
| | | hlrprdlisninvivlelkgsettfmceyadetativeflnrwitfcqsiistltSGGPGPAGMKGLPGSE |
| | | VQLVESGGGLVQPGNSLRLSCAASGFTFSKFGMSWVRQAPGKGLEWVSS |
| | | ISGSGRDTLYAESVKGRFTISRDNAKTTLYLQMNSLRPEDTAVYYCTIGG |
| | | SLSVSSQGTLVTVSSggggsggggsggggsggggsggggsggggsSGGPGPAGMKGLP |
| | | GSEVQLVESGGGLVQPGGSLRLSCAASGFTFSSYTLAWVRQAPGKGLEW |
| | | VAAIDSSSYTYSPDTVRGRFTISRDNAKNSLYLQMNSLRAEDTAVYYCAR |
| | | DSNWDALDYWGQGTTVTVSSGGGGSGGGGSGGGGSDIQMTQSPSSLSA |
| | | SVGDRVTITCKASQNVGTNVGWYQQKPGKAPKALIYSASFRYSGVPSRF |
| | | SGSGSGTDFTLTISSLQPEDFATYYCQQYYTYPYTFGGGTKVEIKSGGPGP |
| | | AGMKGLPGSggggsggggsggggsggggsggggsggggselcdddppeiphatfkamaykegtml |
| | | nceckrgfrriksgslymlctgnsshsswdnqcqctssatrnttkqvtpqpeeqkerkttemqspmqpvdqasl |
| | | pghcrepppweneateriyhfvvgqmvyyqcvqgyralhrgpaesvckmthgktrwtqpqlictgemetsq |
| | | fpgeekpgaspegrpesetsclvtttdfqiqtemaatmetsiftteyqHHHHHH** |
| 384 | ACP305 | elcdddppeiphatfkamaykegtmlnceckrgfrriksgslymlctgnsshsswdnqcqctssatrnttkqvtpqpeeqk |
| | | erkttemqspmqpvdqaslpghcrepppweneateriyhfvvgqmvyyqcvqgyralhrgpaesvckmthgktrwtq |
| | | pqlictgemetsqfpgeekpqaspegrpesetsclvtttdfqiqtemaatmetsiftteyqggggsggggsggggsggggsg |
| | | gggsggggsSGGPGPAGMKGLPGSaptssstkktqlqlehllldlqmilnginnyknpkltrmltfkfympkka |
| | | telkhlqcleeelkpleevlnlaqsknfhlrprdlisninvivlelkgsettfmceyadetativeflnrwitfcqsiistltSGGP |
| | | GPAGMKGLPGSEVQLVESGGGLVQPGNSLRLSCAASGFTFSKFGMSWVRQAPG |
| | | KGLEWVSSISGSGRDTLYAESVKGRFTISRDNAKTTLYLQMNSLRPEDTAVYYC |
| | | TIGGSLSVSSQGTLVTVSSggggsggggsggggsggggsggggsggggsSGGPGPAGMKGLPG |
| | | SEVQLVESGGGLVQPGGSLRLSCAASGFTFSSYTLAWVRQAPGKGLEWVAAIDS |
| | | SSYTYSPDTVRGRFTISRDNAKNSLYLQMNSLRAEDTAVYYCARDSNWDALDY |
| | | WGQGTTVTVSSGGGGSGGGGSGGGGSDIQMTQSPSSLSASVGDRVTITCKASQN |
| | | VGTNVGWYQQKPGKAPKALIYSASFRYSGVPSRFSGSGSGTDFTLTISSLQPEDF |
| | | ATYYCQQYYTYPYTFGGGTKVEIKHHHHHH** |
| 385 | ACP306 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltSGGPGPAGMKGLPGSggggsggggsggggs |
| | | ggggsggggsggggselcdddppeiphatfkamaykegtmlnceckrgfrriksgslymlctgnsshsswdnqcqctss |
| | | atrnttkqvtpqpeeqkerkttemqspmqpvdqaslpghcrepppweneateriyhfvvgqmvyyqcvqgyralhrgp |
| | | aesvckmthgktrwtqpqlictgemetsqfpgeekpqaspegrpesetsclvtttdfqiqtemaatmetsiftteyqSGGP |
| | | GPAGMKGLPGSEVQLVESGGGLVQPGNSLRLSCAASGFTFSKFGMSWVRQAPG |
| | | KGLEWVSSISGSGRDTLYAESVKGRFTISRDNAKTTLYLQMNSLRPEDTAVYYC |
| | | TIGGSLSVSSQGTLVTVSSggggsggggsggggsggggsggggsggggsSGGPGPAGMKGLPG |
| | | SEVQLVESGGGLVQPGGSLRLSCAASGFTFSSYTLAWVRQAPGKGLEWVAAIDS |
| | | SSYTYSPDTVRGRFTISRDNAKNSLYLQMNSLRAEDTAVYYCARDSNWDALDY |
| | | WGQGTTVTVSSGGGGSGGGGSGGGGSDIQMTQSPSSLSASVGDRVTITCKASQN |
| | | VGTNVGWYQQKPGKAPKALIYSASFRYSGVPSRFSGSGSGTDFTLTISSLQPEDF |
| | | ATYYCQQYYTYPYTFGGGTKVEIKHHHHHH** |
| 386 | ACP307 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSSGGPGPAGMKGLPGStfkfympkkatelkhlqcleeelkpleevlnlaqsknfhlrprdlisninvi |
| | | vlelkgsettfmceyadetativeflnrwitfcqsiistltGGssstkktqlqlehllldlqmilnginnyknpkltrmlSGGP |
| | | GPAGMKGLPGSEVQLVESGGGLVQPGNSLRLSCAASGFTFSKFGMSWVRQAPG |
| | | KGLEWVSSISGSGRDTLYAESVKGRFTISRDNAKTTLYLQMNSLRPEDTAVYYC |
| | | TIGGSLSVSSQGTLVTVSSggggsggggsggggsggggsggggsggggsSGGPGPAGMKGLPG |
| | | SEVQLVESGGGLVQPGGSLRLSCAASGFTFSSYTLAWVRQAPGKGLEWVAAIDS |
| | | SSYTYSPDTVRGRFTISRDNAKNSLYLQMNSLRAEDTAVYYCARDSNWDALDY |
| | | WGQGTTVTVSSGGGGSGGGGSGGGGSDIQMTQSPSSLSASVGDRVTITCKASQN |
| | | VGTNVGWYQQKPGKAPKALIYSASFRYSGVPSRFSGSGSGTDFTLTISSLQPEDF |
| | | ATYYCQQYYTYPYTFGGGTKVEIKHHHHHH** |
| 387 | ACP308 | EVQLVESGGGLVQPGGSLRLSCAASGFTFSSYTLAWVRQAPGKGLEWVAAIDSS |
| | | SYTYSPDTVRGRFTISRDNAKNSLYLQMNSLRAEDTAVYYCARDSNWDALDYW |
| | | GQGTTVTVSSGGGGSGGGGSGGGGSDIQMTQSPSSLSASVGDRVTITCKASQNV |
| | | GTNVGWYQQKPGKAPKALIYSASFRYSGVPSRFSGSGSGTDFTLTISSLQPEDFA |
| | | TYYCQQYYTYPYTFGGGTKVEIKSGGPGPAGMKGLPGSggggsggggsggggsggggsg |
| | | gggsggggsEVQLVESGGGLVQPGNSLRLSCAASGFTFSKFGMSWVRQAPGKGLE |
| | | WVSSISGSGRDTLYAESVKGRFTISRDNAKTTLYLQMNSLRPEDTAVYYCTIGGS |
| | | LSVSSQGTLVTVSSSGGPGPAGMKGLPGStfkfympkkatelkhlqcleeelkpleevlnlaqsknfhl |
| | | rprdlisninvivlelkgsettfmceyadetativeflnrwitfcqsiistltGGssstkktqlqlehllldlqmilnginnyknpk |
| | | ltrmlSGGPGPAGMKGLPGSEVQLVESGGGLVQPGNSLRLSCAASGFTFSKFGMS |
| | | WVRQAPGKGLEWVSSISGSGRDTLYAESVKGRFTISRDNAKTTLYLQMNSLRPE |
| | | DTAVYYCTIGGSLSVSSQGTLVTVSSHHHHHH** |
| 388 | ACP309 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltSGGPGPAGMKGLPGSEVQLVESGGG |
| | | LVQPGNSLRLSCAASGFTFSKFGMSWVRQAPGKGLEWVSSISGSGRDTLYAESV |
| | | KGRFTISRDNAKTTLYLQMNSLRPEDTAVYYCTIGGSLSVSSQGTLVTVSSggggsg |
| | | gggsggggsggggsggggsggggsSGGPGPAGMKGLPGSEVQLVESGGGLVQPGGSLRLS |
| | | CAASGFTFSSYTLAWVRQAPGKGLEWVAAIDSSSYTYSPDTVRGRFTISRDNAK |
| | | NSLYLQMNSLRAEDTAVYYCARDSNWDALDYWGQGTTVTVSSGGGGSGGGGS |
| | | GGGGSDIQMTQSPSSLSASVGDRVTITCKASQNVGTNVGWYQQKPGKAPKSLIY |
| | | SASFRYSGVPSRFSGSGSGTDFTLTISSLQPEDFATYYCQQYYTYPYTFGGGTKV |
| | | EIKHHHHHH** |
| 389 | ACP310 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltSGGPGPAGMKGLPGSEVQLVESGGG |
| | | LVQPGNSLRLSCAASGFTFSKFGMSWVRQAPGKGLEWVSSISGSGRDTLYAESV |
| | | KGRFTISRDNAKTTLYLQMNSLRPEDTAVYYCTIGGSLSVSSQGTLVTVSSggggsg |
| | | gggsggggsggggsggggsggggsSGGPGPAGMKGLPGSEVQLVESGGGLVQPGGSLRLS |
| | | CAASGFTFSSYTLAWVRQAPGKGLEWVAAIDSSSYTYSPDTVRGRFTISRDNAK |
| | | NSLYLQMNSLRAEDTAVYYCARDSNWDALDYWGQGTTVTVSSGGGGSGGGGS |
| | | GGGGSDIQMTQSPSSLSASVGDRVTITCKASQNVGTNVGWYQQKPGQAPRLLIY |
| | | SASFRYSGVPSRFSGSGSGTDFTLTISSLQPEDFATYYCQQYYTYPYTFGGGTKV |
| | | EIKHHHHHH** |
| 390 | ACP311 | aptssstkktqlqlehllldlqmilnginnyknpkltrmltfldympkkatelkhlqcleeelkpleevlnlaqsknf |
| | | hlrprdlisninvivlelkgsettfmceyadetativeflnrwitfcqsiistltSGGPGPAGMKGLPGSes |
| | | kygppcppcpapeflggpsvflfppkpkdtlmisrtpevtcvvvdvsqedpevqfnwyvdgvevhnaktkpr |
| | | eeqfnstyrvvsvltvlhqdwlngkeykckvsnkglpssiektiskakgqprepqvytlppsqeemtknqvslt |
| | | clvkgfypsdiavewesngqpennykttppvldsdgsfflysrltvdksrwqegnvfscsvmhealhnhytqk |
| | | slslslgkggggsggggsggggsggggsggggsggggsSGGPGPAGMKGLPGSEVQLVESG |
| | | GGLVQPGGSLRLSCAASGFTFSSYTLAWVRQAPGKGLEWVAAIDSSSYT |
| | | YSPDTVRGRFTISRDNAKNSLYLQMNSLRAEDTAVYYCARDSNWDALD |
| | | YWGQGTTVTVSSGGGGSGGGGSGGGGSDIQMTQSPSSLSASVGDRVTIT |
| | | CKASQNVGTNVGWYQQKPGKAPKALIYSASFRYSGVPSRFSGSGSGTDF |
| | | TLTISSLQPEDFATYYCQQYYTYPYTFGGGTKVEIKHHHHHHH** |
| 391 | ACP312 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgkSGGPGPA |
| | | GMKGLPGSaptssstkktqlqlehllldlqmilnginnyknpkltrmlafympkkatelkhlqcleeelkpleevlnl |
| | | aqsknfhlrprdlisninvivlelkgsettfmceyadetativeflnrwitfcqsiistltggggsggggsggggsggggsgggg |
| | | sggggsSGGPGPAGMKGLPGSEVQLVESGGGLVQPGGSLRLSCAASGFTFSSYTLA |
| | | WVRQAPGKGLEWVAAIDSSSYTYSPDTVRGRFTISRDNAKNSLYLQMNSLRAE |
| | | DTAVYYCARDSNWDALDYWGQGTTVTVSSGGGGSGGGGSGGGGSDIQMTQSP |
| | | SSLSASVGDRVTITCKASQNVGTNVGWYQQKPGKAPKALIYSASFRYSGVPSRF |
| | | SGSGSGTDFTLTISSLQPEDFATYYCQQYYTYPYTFGGGTKVEIKHHHHHH** |
| 392 | ACP313 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltSGGPGPAGMKGLPGSggggsggggsggggs |
| | | ggggsggggsggggsEVQLVESGGGLVQPGGSLRLSCAASGFTFSSYTLAWVRQAPGK |
| | | GLEWVAAIDSSSYTYSPDTVRGRFTISRDNAKNSLYLQMNSLRAEDTAVYYCAR |
| | | DSNWDALDYWGQGTTVTVSSGGGGSGGGGSGGGGSDIQMTQSPSSLSASVGDR |
| | | VTITCKASQNVGTNVGWYQQKPGKAPKALIYSASFRYSGVPSRFSGSGSGTDFT |
| | | LTISSLQPEDFATYYCQQYYTYPYTFGGGTKVEIKSGGPGPAGMKGLPGSeskygpp |
| | | cppcpapeflggpsvflfppkpkdtlmisrtpevtcvvvdvsqedpevqfnwyvdgvevhnaktkpreeqfnstyrvvs |
| | | vltvlhqdwlngkeykckvsnkglpssiektiskakgqprepqvytlppsqeemtknqvsltclvkgfypsdiavewesn |
| | | gqpennykappvldsdgsfflysrltvdksrwqegnvfscsvmhealhnhytqkslslslgkHHHHHH** |
| 393 | ACP314 | vprdcgckpcictvpevssvfifppkpkdvltitltpkvtcvvvdiskddpevqfswfvddvevhtaqtqpreeq |
| | | fnstfrsvselpimhqdwlngkefkcrvnsaafpapiektisktkgrpkapqvytipppkeqmakdkvsltcmi |
| | | tdffpeditvewqwngqpaenykntqpimdtdgsyfvysklnvqksnweagntftcsvlheglhnhhteksls |
| | | hspgkSGGPGPAGMKGLPGSaptssstkktqlqlehllldlqmilnginnyknpkltrmltfldympk |
| | | katelkhlqcleeelkpleevlnlaqsknfhlrprdlisninvivlelkgsettfmceyadetativeflnrwitfcqsii |
| | | stltggggsggggsggggsggggsggggsggggsSGGPGPAGMKGLPGSEVQLVESGGG |
| | | LVQPGGSLRLSCAASGFTFSSYTLAWVRQAPGKGLEWVAAIDSSSYTYSP |
| | | DTVRGRFTISRDNAKNSLYLQMNSLRAEDTAVYYCARDSNWDALDYWG |
| | | QGTTVTVSSGGGGSGGGGSGGGGSDIQMTQSPSSLSASVGDRVTITCKAS |
| | | QNVGTNVGWYQQKPGKAPKALIYSASFRYSGVPSRFSGSGSGTDFTLTIS |
| | | SLQPEDFATYYCQQYYTYPYTFGGGTKVEIKHHHHHH** |
| 394 | ACP336 | aptssstkktqlqlehllldlqmilnginnyknpkltrmltfidympkkatelkhlqcleeelkpleevlnlaqsknf |
| | | hlrprdlisninvivlelkgsettfmceyadetativeflnrwitfcqsiistltsggpGPAGLYAQpgsEVQ |
| | | LVESGGGLVQPGNSLRLSCAASGFTFSKFGMSWVRQAPGKGLEWVSSIS |
| | | GSGRDTLYAESVKGRFTISRDNAKTTLYLQMNSLRPEDTAVYYCTIGGSL |
| | | SVSSQGTLVTVSSggggsggggsggggsggggsggggsggggssggpGPAGLYAQpgsEV |
| | | QLVESGGGLVQPGGSLRLSCAASGFTFSSYTLAWVRQAPGKGLEWVAAI |
| | | DSSSYTYSPDTVRGRFTISRDNAKNSLYLQMNSLRAEDTAVYYCARDSN |
| | | WDALDYWGQGTTVTVSSsggpGPAGLYAQpgsDIQMTQSPSSLSASVGDR |
| | | VTITCKAREKLWSAVAWYQQKPGKAPKsLIYSASFRYSGVPSRFSGSGSG |
| | | TDFTLTISSLQPEDFATYYCQQYYTYPYTFGGGTKVEIK** |
| 395 | ACP337 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGQGTTVTVSSGGGGSGGGGSGGG |
| | | GSDIQMTQSPSSLSASVGDRVTITCKAREKLWSAVAWYQQKPGKAPKsLIYSASF |
| | | RYSGVPSRFSGSGSGTDFTLTISSLQPEDFATYYCQQYYTYPYTFGGGTKVEIK** |
| 396 | ACP338 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGQGTTVTVSSsggpGPAGLYAQpgsD |
| | | IQMTQSPSSLSASVGDRVTITCKVTEKVWGNVAWYQQKPGKAPISLIYSPSLRKS |
| | | GVPSRFSGSGSGTDFTLTISSLQPEDFATYYCQQYYTYPYTFGGGTKVEIK** |
| 397 | ACP339 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsEVQLVESGGGLVQPGGSLRLSCAAS |
| | | GFTFSSYTLAWVRQAPGKGLEWVAAIDSSSYTYSPDTVRGRFTISRDNAKNSLY |
| | | LQMNSLRAEDTAVYYCARDSNWDALDYWGQGTTVTVSSGGGGSGGGGSGGG |
| | | GSDIQMTQSPSSLSASVGDRVTITCKVTEKVWGNVAWYQQKPGKAPISLIYSPSL |
| | | RKSGVPSRFSGSGSGTDFTLTISSLQPEDFATYYCQQYYTYPYTFGGGTKVEIK** |
| 398 | ACP340 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsevqllesggglvqpggslrlscaasgsifsanamgwyrq |
| | | apgkglelvavissggstnyadsvkgrftisrdnskntvylqmnslraedtavyycmysgsyyytpndywgqgtlvtvss* |
| | | * |
| 399 | ACP341 | aptssstkktqlqlehllldlqmilnginnyknpkltrmltfkfympkkatelkhlqcleeelkpleevlnlaqsknfhlrprdli |
| | | sninvivlelkgsettfmceyadetativeflnrwitfcqsiistltsggpGPAGLYAQpgsEVQLVESGGGLV |
| | | QPGNSLRLSCAASGFTFSKFGMSWVRQAPGKGLEWVSSISGSGRDTLYAESVKG |
| | | RFTISRDNAKTTLYLQMNSLRPEDTAVYYCTIGGSLSVSSQGTLVTVSSggggsgggg |
| | | sggggsggggsggggsggggssggpGPAGLYAQpgsevqllesggglvqpggslrlscaaserifstdvmgwyrq |
| | | apgkqrelvavvsargttnyldavkgrftisrdnskntlylqmnslraedtavyycyvrettspwriywgqgtlvtvss** |
| 400 | ACP342 | elcdddppeiphatfkamaykegtmlnceckrgfrriksgslymlctgnsshsswdnqcqctssatrnttkqvtpqpeeqk |
| | | erkttemqspmqpvdqaslpghcrepppweneateriyhfvvgqmvyyqcvqgyralhrgpaesvckmthgktrwtq |
| | | pqlictgemetsqfpgeekpqaspegrpesetsSlvtttdfqiqtemaatmetsiftteyqggggsggggsggggsggggs |
| | | ggggsggggssggpGPAGLYAQpgsaptssstkktqlqlehllldlqmilnginnyknpkltrmltfkfympkkatel |
| | | khlqcleeelkpleevlnlaqsknfhlrprdlisninvivlelkgsettfmceyadetativeflnrwitfcqsiistltsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsEVQL |
| | | VESGGGLVQPGGSLRLSCAASGFTFSSYTLAWVRQAPGKGLEWVAAIDSSSYTY |
| | | SPDTVRGRFTISRDNAKNSLYLQMNSLRAEDTAVYYCARDSNWDALDYWGQG |
| | | TTVTVSSsggpGPAGLYAQpgsDIQMTQSPSSLSASVGDRVTITCKAREKLWSAVA |
| | | WYQQKPGKAPKsLIYSASFRYSGVPSRFSGSGSGTDFTLTISSLQPEDFATYYCQQ |
| | | YYTYPYTFGGGTKVEIK** |
| 401 | ACP343 | elcdddppeiphatfkamaykegtmlnceckrgfrriksgslymlctgnsshsswdnqcqctssatrnttkqvtpqpeeqk |
| | | erkttemqspmqpvdqaslpghcrepppweneateriyhfvvgqmvyyqcvqgyralhrgpaesvckmthgktrwtq |
| | | pqlictgemetsqfpgeekpqaspegrpesetsSlvtttdfqiqtemaatmetsiftteyqggggsggggsggggsggggs |
| | | ggggsggggssggpGPAGLYAQpgsaptssstkktqlqlehllldlqmilnginnyknpkltrmltfkfympkkatel |
| | | khlqcleeelkpleevlnlaqsknfhlrprdlisninvivlelkgsettfmceyadetativeflnrwitfcqsiistltsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsEVQL |
| | | VESGGGLVQPGGSLRLSCAASGFTFSSYTLAWVRQAPGKGLEWVAAIDSSSYTY |
| | | SPDTVRGRFTISRDNAKNSLYLQMNSLRAEDTAVYYCARDSNWDALDYWGQG |
| | | TTVTVSSGGGGSGGGGSGGGGSDIQMTQSPSSLSASVGDRVTITCKAREKLWSA |
| | | VAWYQQKPGKAPKsLIYSASFRYSGVPSRFSGSGSGTDFTLTISSLQPEDFATYYC |
| | | QQYYTYPYTFGGGTKVEIK** |
| 402 | ACP344 | elcdddppeiphatfkamaykegtmlnceckrgfrriksgslymlctgnsshsswdnqcqctssatrnttkqvtpqpeeqk |
| | | erkttemqspmqpvdqaslpghcrepppweneateriyhfvvgqmvyyqcvqgyralhrgpaesvckmthgktrwtq |
| | | pqlictgemetsqfpgeekpqaspegrpesetsSlvtttdfqiqtemaatmetsiftteyqggggsggggsggggsggggs |
| | | ggggsggggssggpGPAGLYAQpgsaptssstkktqlqlehllldlqmilnginnyknpkltrmltfkfympkkatel |
| | | khlqcleeelkpleevlnlaqsknfhlrprdlisninvivlelkgsettfmceyadetativeflnrwitfcqsiistltsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsEVQL |
| | | VESGGGLVQPGGSLRLSCAASGFTFSSYTLAWVRQAPGKGLEWVAAIDSSSYTY |
| | | SPDTVRGRFTISRDNAKNSLYLQMNSLRAEDTAVYYCARDSNWDALDYWGQG |
| | | TTVTVSSsggpGPAGLYAQpgsDIQMTQSPSSLSASVGDRVTITCKVTEKVWGNVA |
| | | WYQQKPGKAPISLIYSPSLRKSGVPSRFSGSGSGTDFTLTISSLQPEDFATYYCQQ |
| | | YYTYPYTFGGGTKVEIK** |
| 403 | ACP345 | elcdddppeiphatfkamaykegtmlnceckrgfrriksgslymlctgnsshsswdnqcqctssatrnttkqvtpqpeeqk |
| | | erkttemqspmqpvdqaslpghcrepppweneateriyhfvvgqmvyyqcvqgyralhrgpaesvckmthgktrwtq |
| | | pqlictgemetsqfpgeekpqaspegrpesetsSlvtttdfqiqtemaatmetsiftteyqggggsggggsggggsggggs |
| | | ggggsggggssggpGPAGLYAQpgsaptssstkktqlqlehllldlqmilnginnyknpkltrmltfkfympkkatel |
| | | khlqcleeelkpleevlnlaqsknfhlrprdlisninvivlelkgsettfmceyadetativeflnrwitfcqsiistltsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsEVQL |
| | | VESGGGLVQPGGSLRLSCAASGFTFSSYTLAWVRQAPGKGLEWVAAIDSSSYTY |
| | | SPDTVRGRFTISRDNAKNSLYLQMNSLRAEDTAVYYCARDSNWDALDYWGQG |
| | | TTVTVSSGGGGSGGGGSGGGGSDIQMTQSPSSLSASVGDRVTITCKVTEKVWGN |
| | | VAWYQQKPGKAPISLIYSPSLRKSGVPSRFSGSGSGTDFTLTISSLQPEDFATYYC |
| | | QQYYTYPYTFGGGTKVEIK** |
| 404 | ACP346 | elcdddppeiphatfkamaykegtmlnceckrgfrriksgslymlctgnsshsswdnqcqctssatrnttkqvtpqpeeqk |
| | | erkttemqspmqpvdqaslpghcrepppweneateriyhfvvgqmvyyqcvqgyralhrgpaesvckmthgktrwtq |
| | | pqlictgemetsqfpgeekpqaspegrpesetsSlvtttdfqiqtemaatmetsiftteyqggggsggggsggggsggggs |
| | | ggggsggggssggpGPAGLYAQpgsaptssstkktqlqlehllldlqmilnginnyknpkltrmltfkfympkkatel |
| | | khlqcleeelkpleevlnlaqsknfhlrprdlisninvivlelkgsettfmceyadetativeflnrwitfcqsiistltsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsevqlles |
| | | ggglvqpggslrlscaasgsifsanamgwyrqapgkglelvavissggstnyadsvkgrftisrdnskntvylqmnslraed |
| | | tavyycmysgsyyytpndywgqgtlvtvss** |
| 405 | ACP347 | elcdddppeiphatfkamaykegtmlnceckrgfrriksgslymlctgnsshsswdnqcqctssatrnttkqvtpqpeeqk |
| | | erkttemqspmqpvdqaslpghcrepppweneateriyhfvvgqmvyyqcvqgyralhrgpaesvckmthgktrwtq |
| | | pqlictgemetsqfpgeekpqaspegrpesetsSlvtttdfqiqtemaatmetsiftteyqggggsggggsggggsggggs |
| | | ggggsggggssggpGPAGLYAQpgsaptssstkktqlqlehllldlqmilnginnyknpkltrmltfkfympkkatel |
| | | khlqcleeelkpleevlnlaqsknfhlrprdlisninvivlelkgsettfmceyadetativeflnrwitfcqsiistltsggpGP |
| | | AGLYAQpgsEVQLVESGGGLVQPGNSLRLSCAASGFTFSKFGMSWVRQAPGKGL |
| | | EWVSSISGSGRDTLYAESVKGRFTISRDNAKTTLYLQMNSLRPEDTAVYYCTIGG |
| | | SLSVSSQGTLVTVSSggggsggggsggggsggggsggggsggggssggpGPAGLYAQpgsevqlles |
| | | ggglyqpggslrlscaaserifstdvmgwyrqapgkqrelvavvsargttnyldavkgrftisrdnskntlylqmnslraedt |
| | | avyycyvrettspwriywgqgtlvtvss** |
| 406 | ACP348 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKGLEWVAAIDSSSYTYSPDTVRGRFTISRDNAKNSLYLQMNSLRAEDTAV |
| | | YYCARDSNWDALDYWGQGTTVTVSSsggpGPAGLYAQpgsDIQMTQSPSSLSASV |
| | | GDRVTITCKAREKLWSAVAWYQQKPGKAPKsLIYSASFRYSGVPSRFSGSGSGT |
| | | DFTLTISSLQPEDFATYYCQQYYTYPYTFGGGTKVEIK** |
| 407 | ACP349 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclykgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKGLEWVAAIDSSSYTYSPDTVRGRFTISRDNAKNSLYLQMNSLRAEDTAV |
| | | YYCARDSNWDALDYWGQGTTVTVSSGGGGSGGGGSGGGGSDIQMTQSPSSLS |
| | | ASVGDRVTITCKAREKLWSAVAWYQQKPGKAPKsLIYSASFRYSGVPSRFSGSG |
| | | SGTDFTLTISSLQPEDFATYYCQQYYTYPYTFGGGTKVEIK** |
| 408 | ACP350 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKGLEWVAAIDSSSYTYSPDTVRGRFTISRDNAKNSLYLQMNSLRAEDTAV |
| | | YYCARDSNWDALDYWGQGTTVTVSSsggpGPAGLYAQpgsDIQMTQSPSSLSASV |
| | | GDRVTITCKVTEKVWGNVAWYQQKPGKAPISLIYSPSLRKSGVPSRFSGSGSGT |
| | | DFTLTISSLQPEDFATYYCQQYYTYPYTFGGGTKVEIK** |
| 409 | ACP351 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsEVQLVESGGGLVQPGGSLRLSCAASGFTFSSYTLAWVR |
| | | QAPGKGLEWVAAIDSSSYTYSPDTVRGRFTISRDNAKNSLYLQMNSLRAEDTAV |
| | | YYCARDSNWDALDYWGQGTTVTVSSGGGGSGGGGSGGGGSDIQMTQSPSSLS |
| | | ASVGDRVTITCKVTEKVWGNVAWYQQKPGKAPISLIYSPSLRKSGVPSRFSGSGS |
| | | GTDFTLTISSLQPEDFATYYCQQYYTYPYTFGGGTKVEIK** |
| 410 | ACP352 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsevqllesggglvqpggslrlscaasgsifsanamgwyrqapgkglelvavissggst |
| | | nyadsvkgrftisrdnskntvylqmnslraedtavyycmysgsyyytpndywgqgtlvtvss** |
| 411 | ACP353 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgsaptssstkktqlqlehllldlqmilnginnyknpkltrmltfkfympkkatelkhlqcleeelkpleevlnlaqs |
| | | knfhlrprdlisninvivlelkgsettfmceyadetativeflnrwitfcqsiistltggggsggggsggggsggggsggggsg |
| | | gggssggpGPAGLYAQpgsevqllesggglvqpggslrlscaaserifstdvmgwyrqapgkqrelvavvsargttn |
| | | yldavkgrftisrdnskntlylqmnslraedtavyycyvrettspwriywgqgtlvtvss** |
| 412 | ACP354 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgselcdddppeiphatfkamaykegtmlnceckrgfrriksgslymlctgnsshsswdnqcqctssatrnttkq |
| | | vtpqpeeqkerkttemqspmqpvdqaslpghcrepppweneateriyhfvvgqmvyyqcvqgyralhrgpaesvck |
| | | mthgktrwtqpqlictgemetsqfpgeekpqaspegrpesetsSlvtttdfqiqtemaatmetsiftteyqggggsggggsg |
| | | gggsggggsggggsggggssggpGPAGLYAQpgsaptssstkktqlqlehllldlqmilnginnyknpkltrmltfk |
| | | fympkkatelkhlqcleeelkpleevlnlaqsknfhlrprdlisninvivlelkgsettfmceyadetativeflnrwitfcqsiis |
| | | tltggggsggggsggggsggggsggggsggggssggpGPAGLYAQpgsEVQLVESGGGLVQPGGS |
| | | LRLSCAASGFTFSSYTLAWVRQAPGKGLEWVAAIDSSSYTYSPDTVRGRFTISRD |
| | | NAKNSLYLQMNSLRAEDTAVYYCARDSNWDALDYWGQGTTVTVSSsggpGPAG |
| | | LYAQpgsDIQMTQSPSSLSASVGDRVTITCKAREKLWSAVAWYQQKPGKAPKsLI |
| | | YSASFRYSGVPSRFSGSGSGTDFTLTISSLQPEDFATYYCQQYYTYPYTFGGGTK |
| | | VEIK** |
| 413 | ACP355 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgselcdddppeiphatfkamaykegtmlnceckrgfrriksgslymlctgnsshsswdnqcqctssatmttkq |
| | | vtpqpeeqkerkttemqspmqpvdqaslpghcrepppweneateriyhfvvgqmvyyqcvqgyralhrgpaesvck |
| | | mthgktrwtqpqlictgemetsqfpgeekpqaspegrpesetsSlvtttdfqiqtemaatmetsiftteyqggggsggggsg |
| | | gggsggggsggggsggggssggpGPAGLYAQpgsaptssstkktqlqlehllldlqmilnginnyknpkltrmltfk |
| | | fympkkatelkhlqcleeelkpleevlnlaqsknfhlrprdlisninvivlelkgsettfmceyadetativeflnrwitfcqsiis |
| | | tltggggsggggsggggsggggsggggsggggssggpGPAGLYAQpgsEVQLVESGGGLVQPGGS |
| | | LRLSCAASGFTFSSYTLAWVRQAPGKGLEWVAAIDSSSYTYSPDTVRGRFTISRD |
| | | NAKNSLYLQMNSLRAEDTAVYYCARDSNWDALDYWGQGTTVTVSSGGGGSG |
| | | GGGSGGGGSDIQMTQSPSSLSASVGDRVTITCKAREKLWSAVAWYQQKPGKAP |
| | | KsLIYSASFRYSGVPSRFSGSGSGTDFTLTISSLQPEDFATYYCQQYYTYPYTFGG |
| | | GTKVEIK** |
| 414 | ACP356 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgselcdddppeiphatfkamaykegtmlnceckrgfrriksgslymlctgnsshsswdnqcqctssatrnttkq |
| | | vtpqpeeqkerkttemqspmqpvdqaslpghcrepppweneateriyhfvvgqmvyyqcvqgyralhrgpaesyck |
| | | mthgktrwtqpqlictgemetsqfpgeekpqaspegrpesetsSlvtttdfqiqtemaatmetsiftteyqggggsggggsg |
| | | gggsggggsggggsggggssggpGPAGLYAQpgsaptssstkktqlqlehllldlqmilnginnyknpkltrmltfk |
| | | fympkkatelkhlqcleeelkpleevlnlaqsknfhlrprdlisninvivlelkgsettfmceyadetativeflnrwitfcqsiis |
| | | tltggggsggggsggggsggggsggggsggggssggpGPAGLYAQpgsEVQLVESGGGLVQPGGS |
| | | LRLSCAASGFTFSSYTLAWVRQAPGKGLEWVAAIDSSSYTYSPDTVRGRFTISRD |
| | | NAKNSLYLQMNSLRAEDTAVYYCARDSNWDALDYWGQGTTVTVSSsggpGPAG |
| | | LYAQpgsDIQMTQSPSSLSASVGDRVTITCKVTEKVWGNVAWYQQKPGKAPISLI |
| | | YSPSLRKSGVPSRFSGSGSGTDFTLTISSLQPEDFATYYCQQYYTYPYTFGGGTK |
| | | VEIK** |
| 415 | ACP357 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclykgfypsdi |
| | | avewesngqpennykttppyldsdgsfflysrltvdksrwqegnyfscsvmhealhnhytqkslslslgksggpGPAG |
| | | LYAQpgselcdddppeiphatfkamaykegtmlnceckrgfrriksgslymlctgnsshsswdnqcqctssatrnttkq |
| | | vtpqpeeqkerkttemqspmqpvdqaslpghcrepppweneateriyhfvvgqmvyyqcvqgyralhrgpaesvck |
| | | mthgktrwtqpqlictgemetsqfpgeekpqaspegrpesetsSlvtttdfqiqtemaatmetsiftteyqggggsggggsg |
| | | gggsggggsggggsggggssggpGPAGLYAQpgsaptssstkktqlqlehllldlqmilnginnyknpkltrmltfk |
| | | fympkkatelkhlqcleeelkpleevlnlaqsknfhlrprdlisninvivlelkgsettfmceyadetativeflnrwitfcqsiis |
| | | tltggggsggggsggggsggggsggggsggggssggpGPAGLYAQpgsEVQLVESGGGLVQPGGS |
| | | LRLSCAASGFTFSSYTLAWVRQAPGKGLEWVAAIDSSSYTYSPDTVRGRFTISRD |
| | | NAKNSLYLQMNSLRAEDTAVYYCARDSNWDALDYWGQGTTVTVSSGGGGSG |
| | | GGGSGGGGSDIQMTQSPSSLSASVGDRVTITCKVTEKVWGNVAWYQQKPGKAP |
| | | ISLIYSPSLRKSGVPSRFSGSGSGTDFTLTISSLQPEDFATYYCQQYYTYPYTFGGG |
| | | TKVEIK** |
| 416 | ACP358 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgselcdddppeiphatfkamaykegtmlnceckrgfrriksgslymlctgnsshsswdnqcqctssatrnttkq |
| | | vtpqpeeqkerkttemqspmqpvdqaslpghcrepppweneateriyhfvvgqmvyyqcvqgyralhrgpaesvck |
| | | mthgktrwtqpqlictgemetsqfpgeekpqaspegrpesetsSlvtttdfqiqtemaatmetsiftteyqggggsggggsg |
| | | gggsggggsggggsggggssggpGPAGLYAQpgsaptssstkktqlqlehllldlqmilnginnyknpkltrmltfk |
| | | fympkkatelkhlqcleeelkpleevlnlaqsknfhlrprdlisninvivlelkgsettfmceyadetativeflnrwitfcqsiis |
| | | tltggggsggggsggggsggggsggggsggggssggpGPAGLYAQpgsevqllesggglvqpggslrlscaasgsi |
| | | fsanamgwyrqapgkglelvavissggstnyadsvkgrftisrdnskntvylqmnslraedtavyycmysgsyyytpnd |
| | | ywgqgtlvtvss** |
| 417 | ACP359 | eskygppcppcpapeflggpsvflfppkpkdtlmisrtpevtcvvvdvsqedpevqfnwyvdgvevhnaktkpreeqf |
| | | nstyrvvsvltvlhqdwlngkeykckvsnkglpssiektiskakgqprepqvytlppsqeemtknqvsltclvkgfypsdi |
| | | avewesngqpennykttppvldsdgsfflysrltvdksrwqegnvfscsvmhealhnhytqkslslslgksggpGPAG |
| | | LYAQpgselcdddppeiphatfkamaykegtmlnceckrgfrriksgslymlctgnsshsswdnqcqctssatrnttkq |
| | | vtpqpeeqkerkttemqspmqpvdqaslpghcrepppweneateriyhfvvgqmvyyqcvqgyralhrgpaesvck |
| | | mthgktrwtqpqlictgemetsqfpgeekpqaspegrpesetsSlvtttdfqiqtemaatmetsiftteyqggggsggggsg |
| | | gggsggggsggggsggggssggpGPAGLYAQpgsaptssstkktqlqlehllldlqmilnginnyknpkltrmltfk |
| | | fympkkatelkhlqcleeelkpleevlnlaqsknfhlrprdlisninvivlelkgsettfmceyadetativeflnrwitfcqsiis |
| | | tltggggsggggsggggsggggsggggsggggssggpGPAGLYAQpgsevqllesggglyqpggslrlscaaserif |
| | | stdvmgwyrqapgkqrelvavvsargttnyldavkgrftisrdnskntlylqmnslraedtavyycyvrettspwriywgq |
| | | gtlvtvss** |
| 418 | ACP360 | EVQLVESGGGLVQPGGSLRLSCAASGFTFSSYTLAWVRQAPGKGLEWVAAIDSS |
| | | SYTYSPDTVRGRFTISRDNAKNSLYLQMNSLRAEDTAVYYCARDSNWDALDYW |
| | | GQGTTVTVSSggggsggggsggggsDIQMTQSPSSLSASVGDRVTITCKASQNVGTNV |
| | | GWYQQKPGKAPKsLIYSASFRYSGVPSRFSGSGSGTDFTLTISSLQPEDFATYYCQ |
| | | QYYTYPYTFGGGTKVEIKHHHHHH** |
| 419 | ACP361 | EVQLVESGGGLVQPGGSLRLSCAASGFTFSSYTLAWVRQAPGKGLEWVAAIDSS |
| | | SYTYSPDTVRGRFTISRDNAKNSLYLQMNSLRAEDTAVYYCARDSNWDALDYW |
| | | GQGTTVTVSSsggpGPAGLYAQpgsDIQMTQSPSSLSASVGDRVTITCKASQNVGT |
| | | NVGWYQQKPGKAPKALIYSASFRYSGVPSRFSGSGSGTDFTLTISSLQPEDFATY |
| | | YCQQYYTYPYTFGGGTKVEIKHHHHHH** |
| 420 | ACP362 | EVQLVESGGGLVQPGGSLRLSCAASGFTFSSYTLAWVRQAPGKGLEWVAAIDSS |
| | | SYTYSPDTVRGRFTISRDNAKNSLYLQMNSLRAEDTAVYYCARDSNWDALDYW |
| | | GQGTTVTVSSsggpGPAGLYAQpgsDIQMTQSPSSLSASVGDRVTITCKASQNVGT |
| | | NVGWYQQKPGKAPKsLIYSASFRYSGVPSRFSGSGSGTDFTLTISSLQPEDFATYY |
| | | CQQYYTYPYTFGGGTKVEIKHHHHHH** |
| 421 | ACP200 | lveepknlvktncdlyeklgeygfqnailvrytqkapqvstptlveaarnlgrvgtkcctlpedqrlpcvedylsail |
| | | nrvcllhektpvsehvtkccsgslverrpcfsaltvdetyvpkefkaetftfhsdictlpekekqikkqtalaelvkh |
| | | kpkataeqlktvmddfaqfldtcckaadkdtcfstegpnlvtrckdalaSGGPGPAGMKGLPGScdlp |
| | | qthnlrnkraltllvqmrrlsplsclkdrkdfgfpqekvdaqqikkapipvlseltqqilniftskdssaawnttlld |
| | | sfcndlhqqlndlqgclmqqvgvqefpltqedallavrkyfhritvylrekkhspcawevvraevwralsssan |
| | | vlgrlreekSGGPGPAGMKGLPGSlveepknlvktncdlyeklgeygfqnailvrytqkapqvstptl |
| | | veaarnlgrvgtkcctlpedqrlpcvedylsailnrvcllhektpvsehytkccsgslverrpcfsaltvdetyvpke |
| | | fkaetftfhsdictlpekekqikkqtalaelvkhkpkataeqlktvmddfaqfldtcckaadkdtcfstegpnlvtrc |
| | | kdalaHHHHHH** |
| 422 | ACP201 | eahkseiahryndlgeqhfkglvliafsqylqkcsydehaklvqevtdfaktcvadesaancdkslhtlfgdklcaipnlren |
| | | ygeladcctkqepernecflqhkddnpslppferpeaeamctsfkenpttfmghylhevarrhpyfyapellyyaeqynei |
| | | ltqccaeadkescltpkldgykekalvssyrqGGGGSGGGGSGGSlveepknlvktncdlyeklgeygfqnailvr |
| | | ytqkapqvstptlveaarnlgrvgtkcctlpedqrlpcvedylsailnrycllhektpvsehvtkccsgslverrpcfsaltvdet |
| | | yypkefkaetftfhsdictlpekekqikkqtalaelvkhkpkataeqlktvmddfaqfldtcckaadkdtcfstegpnlvtrck |
| | | dalaSGGPGPAGMKGLPGScdlpqthnlmkraltllvqmrrlsplsclkdrkdfgfpqekvdaqqikkapipvl |
| | | seltqqilniftskdssaawnttlldsfcndlhqqlndlqgclmqqvgvqefpltqedallavrkyfhritvylrekkhspcaw |
| | | evvraevwralsssanvlgrlreekSGGPGPAGMKGLPGSeahkseiahryndlgeqhfkglvliafsqylqkcs |
| | | ydehaklvqevtdfaktcvadesaancdkslhtlfgdklcaipnlrenygeladcctkqepernecflqhkddnpslppfer |
| | | peaeamctsfkenpttfmghylhevarrhpyfyapellyyaeqyneiltqccaeadkescltpkldgykekalvssyrqG |
| | | GGGSGGGGSGGSlveepknlvktncdlyeklgeygfqnailvrytqkapqvstptlveaarnlgrvgtkcctlpedq |
| | | rlpcvedylsailnrvcllhektpvsehvtkccsgslverrpcfsaltvdetyvpkefkaetftfhsdictlpekekqikkqtala |
| | | elvkhkpkataeqlktvmddfaqfldtcckaadkdtcfstegpnlytrckdalaHHHHHH** |
| 423 | ACP202 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSggggsgggSGGPGPAGMKGLPGSggggsgggscdlpqthnlrnkraltllvqmrrlsplsclkdrk |
| | | dfgfpqekvdaqqikkapipvlseltqqilniftskdssaawnttlldsfcndlhqqlndlqgclmqqvgvqefpltqedall |
| | | avrkyfhritvylrekkhspcawevvraevwralsssanvlgrlreekggggsgggSGGPGPAGMKGLPGSgg |
| | | ggsgggsEVQLVESGGGLVQPGNSLRLSCAASGFTFSKFGMSWVRQAPGKGLEWV |
| | | SSISGSGRDTLYAESVKGRFTISRDNAKTTLYLQMNSLRPEDTAVYYCTIGGSLS |
| | | VSSQGTLVTVSSHHHHHH** |
| 424 | ACP203 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpGPAGLYAQpgscdlpqthnlrnkraltllvqmrrlsplsclkdrkdfgfpqekvdaqqikkaqai |
| | | pvlseltqqilniftskdssaawnttlldsfcndlhqqlndlqgclmqqvgvqefpltqedallavrkyfhritvylrekkhspc |
| | | awevvraevwralsssanvlgrlreeksggpGPAGLYAQpgsEVQLVESGGGLVQPGNSLRLSCA |
| | | ASGFTFSKFGMSWVRQAPGKGLEWVSSISGSGRDTLYAESVKGRFTISRDNAKT |
| | | TLYLQMNSLRPEDTAVYYCTIGGSLSVSSQGTLVTVSS** |
| 425 | ACP204 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpALFKSSFPpgscdlpqthnlrnkraltllvqmrrlsplsclkdrkdfgfpqekvdaqqikkaqaipv |
| | | lseltqqilniftskdssaawnttlldsfcndlhqqlndlqgclmqqvgvqefpltqedallavrkyfhritvylrekkhspcaw |
| | | evvraevwralsssanvlgrlreeksggpALFKSSFPpgsEVQLVESGGGLVQPGNSLRLSCAAS |
| | | GFTFSKFGMSWVRQAPGKGLEWVSSISGSGRDTLYAESVKGRFTISRDNAKTTL |
| | | YLQMNSLRPEDTAVYYCTIGGSLSVSSQGTLVTVSS** |
| 426 | ACP205 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpPLAQKLKSSpgscdlpqthnlrnkraltllvqmrrlsplsclkdrkdfgfpqekvdaqqikkaqai |
| | | pvlseltqqilniftskdssaawnttlldsfcndlhqqlndlqgclmqqvgvqefpltqedallavrkyfhritvylrekkhspc |
| | | awevvraevwralsssanvlgrlreeksggpPLAQKLKSSpgsEVQLVESGGGLVQPGNSLRLSC |
| | | AASGFTFSKFGMSWVRQAPGKGLEWVSSISGSGRDTLYAESVKGRFTISRDNAK |
| | | TTLYLQMNSLRPEDTAVYYCTIGGSLSVSSQGTLVTVSS** |
| 427 | ACP206 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpGPAGLYAQpgscdlpqthslgsrrtlmllaqmrrislfsclkdrhdfgfpqeefgnqfqkaetipvl |
| | | hemiqqifnlfstkdssaawdetlldkfytelyqqlndleacviqgvgvtetplmkedsilavrkyfqritlylkekkyspca |
| | | wevvraeimrsfslstnlqeslrskesggpGPAGLYAQpgsEVQLVESGGGLVQPGNSLRLSCAA |
| | | SGFTFSKFGMSWVRQAPGKGLEWVSSISGSGRDTLYAESVKGRFTISRDNAKTT |
| | | LYLQMNSLRPEDTAVYYCTIGGSLSVSSQGTLVTVSS** |
| 428 | ACP207 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpALFKSSFPpgscdlpqthslgsrrtlmllaqmrrislfsclkdrhdfgfpqeefgnqfqkaetipvlh |
| | | emiqqifnlfstkdssaawdetlldkfytelyqqlndleacviqgvgvtetplmkedsilavrkyfqritlylkekkyspcaw |
| | | evvraeimrsfslstnlqeslrskesggpALFKSSFPpgsEVQLVESGGGLVQPGNSLRLSCAASG |
| | | FTFSKFGMSWVRQAPGKGLEWVSSISGSGRDTLYAESVKGRFTISRDNAKTTLY |
| | | LQMNSLRPEDTAVYYCTIGGSLSVSSQGTLVTVSS** |
| 429 | ACP208 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSsggpPLAQKLKSSpgscdlpqthslgsrrtlmllaqmrrislfsclkdrhdfgfpqeefgnqfqkaetipv |
| | | lhemiqqifnlfstkdssaawdetlldkfytelyqqlndleacviqgvgvtetplmkedsilavrkyfqritlylkekkyspca |
| | | wevvraeimrsfslstnlqeslrskesggpPLAQKLKSSpgsEVQLVESGGGLVQPGNSLRLSCAA |
| | | SGFTFSKFGMSWVRQAPGKGLEWVSSISGSGRDTLYAESVKGRFTISRDNAKTT |
| | | LYLQMNSLRPEDTAVYYCTIGGSLSVSSQGTLVTVSS** |
| 430 | ACP211 | EVQLVESGGGLVQPGNSLRLSCAASGFTFSKFGMSWVRQAPGKGLEWVS |
| | | SISGSGRDTLYAESVKGRFTISRDNAKTTLYLQMNSLRPEDTAVYYCTIG |
| | | GSLSVSSQGTLVTVSSSGGPGPAGMKGLPGShgtviesleslnnyfnssgidveekslfldi |
| | | wrnwqkdgdmkilqsqiisfylrlfevlkdnqaisnnisvieshlittffsnskakkdafmsiakfevnnpqvqr |
| | | qafnelirvvhqllpesslrkrkrsrcSGGPGPAGMKGLPGScdlpqthnlrnkraltllvqmrrlsplsc |
| | | lkdrkdfgfpqekvdaqqikkapipvlseltqqilniftskdssaawnttlldsfcndlhqqlndlqgclmqqvg |
| | | vqefpltqedallavrkyfhritvylrekkhspcawevvraevwralsssanvlgrlreekSGGPGPAGM |
| | | KGLPGShgtviesleslnnyfnssgidveekslfldiwrnwqkdgdmkilqsqiisfylrlfevlkdnqaisnn |
| | | isvieshlittffsnskakkdafmsiakfevnnpqvqrqafnelirvvhqllpesslrkrkrsrcSGGPGPAG |
| | | MKGLPGSEVQLVESGGGLVQPGNSLRLSCAASGFTFSKFGMSWVRQAPG |
| | | KGLEWVSSISGSGRDTLYAESVKGRFTISRDNAKTTLYLQMNSLRPEDTA |
| | | VYYCTIGGSLSVSSQGTLVTVSSHEIREIREI |
| 431 | ACP213 | lveepknlvktncdlyeklgeygfqnailvrytqkapqvstptlveaarnlgrvgtkcctlpedqrlpcvedylsail |
| | | nrvcllhektpvsehvtkccsgslverrpcfsaltvdetyvpkefkaetftfhsdictlpekekqikkqtalaelvkh |
| | | kpkataeqlktvmddfaqfldtcckaadkdtcfstegpnlvtrckdalaSGGPGPAGMKGLPGShgt |
| | | viesleslnnyfnssgidveekslfldiwrnwqkdgdmkilqsqiisfylrlfevlkdnqaisnnisvieshlittffs |
| | | nskakkdafmsiakfevnnpqvqrqafnelirvvhqllpesslrkrkrsrcSGGPGPAGMKGLPGSlv |
| | | eepknlvktncdlyeklgeygfqnailvrytqkapqvstptlveaarnlgrvgtkcctlpedqrlpcvedylsailn |
| | | rvcllhektpvsehvtkccsgslverrpcfsaltvdetyvpkefkaetftfhsdictlpekekqikkqtalaelvkhk |
| | | pkataeqlktvmddfaqfldtcckaadkdtcfstegpnlvtrckdalaSGGPGPAGMKGLPGShgtvi |
| | | esleslnnyfnssgidveekslfldiwrnwqkdgdmkilqsqiisfylrlfevlkdnqaisnnisvieshlittffsns |
| | | kakkdafmsiakfevnnpqvqrqafnelirvvhqllpesslrkrkrsrcSGGPGPAGMKGLPGSlvee |
| | | pknlvktncdlyeklgeygfqnailvrytqkapqvstptlveaarnlgrvgtkcctlpedqrlpcvedylsailnrv |
| | | cllhektpvsehvtkccsgslverrpcfsaltvdetyvpkeflcaetftfhsdictlpekekqikkqtalaelvkhkpk |
| | | ataeqlktvmddfaqfldtcckaadkdtcfstegpnlvtrckdalaHHHHHH** |
| 432 | ACP214 | eahkseiahryndlgeqhfkglvliafsqylqkcsydehaklyqevtdfaktcvadesaancdkslhtlfgdklcaipnlren |
| | | ygeladcctkqepernecflqhkddnpslppferpeaeamctsfkenpttfmghylhevarrhpyfyapellyyaeqynei |
| | | ltqccaeadkescltpkldgykekalvssyrqGGGGSGGGGSGGSlveepknlvktncdlyeklgeygfqnailvr |
| | | ytqkapqvstptlveaarnlgrvgtkcctlpedqrlpcvedylsailnrvcllhektpysehvtkccsgslverrpcfsaltvdet |
| | | yypkefkaetftfhsdictlpekekqikkqtalaelvkhkpkataeqlktvmddfaqfldtcckaadkdtcfstegpnlvtrck |
| | | dalaSGGPGPAGMKGLPGShgtviesleslnnyfnssgidveekslfldiwrnwqkdgdmkilqsqiisfylrlfe |
| | | vlkdnqaisnnisvieshlittffsnskakkdafmsiakfevnnpqvqrqafnelirvvhqllpesslrkrkrsrcSGGPGP |
| | | AGMKGLPGSeahkseiahryndlgeqhfkglvliafsqylqkcsydehaklyqevtdfaktcvadesaancdkslht |
| | | lfgdklcaipnlrenygeladcctkqepernecflqhkddnpslppferpeaeamctsfkenpttfmghylhevarrhpyfy |
| | | apellyyaeqyneiltqccaeadkescltpkldgvkekalvssvrqGGGGSGGGGSGGSlveepknlvktncdly |
| | | eklgeygfqnailvrytqkapqvstptlveaarnlgrvgtkcctlpedqrlpcvedylsailnrvcllhektpvsehvtkccsgs |
| | | lverrpcfsaltvdetyypkefkaetftfhsdictlpekekqikkqtalaelvkhkpkataeqlktvmddfaqfldtcckaadk |
| | | dtcfstegpnlvtrckdalaSGGPGPAGMKGLPGShgtviesleslnnyfnssgidveekslfldiwrnwqkdgd |
| | | mkilqsqiisfylrlfeylkdnqaisnnisvieshlittffsnskakkdafnsiakfevnnpqvqrqafnelirvvhqllpesslr |
| | | krkrsrcSGGPGPAGMKGLPGSeahkseiahryndlgeqhfkglvliafsqylqkcsydehaklyqevtdfaktc |
| | | vadesaancdkslhtlfgdklcaipnlrenygeladcctkqepernecflqhkddnpslppferpeaeamctsfkenpttfm |
| | | ghylhevarrhpyfyapellyyaeqyneiltqccaeadkescltpkldgvkekalvssvrqGGGGSGGGGSGGS1 |
| | | veepknlvktncdlyeklgeygfqnailvrytqkapqvstptlveaarnlgrvgtkcctlpedqrlpcvedylsailnrvcllhe |
| | | ktpvsehvtkccsgslverrpcfsaltvdetyvpkefkaetftfhsdictlpekekqikkqtalaelvkhkpkataeqlktvmd |
| | | dfaqfldtcckaadkdtcfstegpnlvtrckdalaHHHHHH** |
| 433 | ACP215 | EVQLVESGGGLVQPGNSLRLSCAASGFTFSKFGMSWVRQAPGKGLEWVSSISGS |
| | | GRDTLYAESVKGRFTISRDNAKTTLYLQMNSLRPEDTAVYYCTIGGSLSVSSQGT |
| | | LVTVSSggggsgggSGGPGPAGMKGLPGSggggsgggshgtviesleslnnyfnssgidveekslfldiwr |
| | | nwqkdgdmkilqsqiisfylrlfevlkdnqaisnnisvieshlittffsnskakkdafmsiakfevnnpqvqrqafnelirvv |
| | | hqllpesslrkrkrsrcggggsgggSGGPGPAGMKGLPGSggggsgggsEVQLVESGGGLVQPGN |
| | | SLRLSCAASGFTFSKFGMSWVRQAPGKGLEWVSSISGSGRDTLYAESVKGRFTIS |
| | | RDNAKTTLYLQMNSLRPEDTAVYYCTIGGSLSVSSQGTLVTVSSggggsgggSGGP |
| | | GPAGMKGLPGSggggsgggshgtviesleslnnyfnssgidveekslfldiwrnwqkdgdmkilqsqiisfylrlfe |
| | | vlkdnqaisnnisvieshlittffsnskakkdafmsiakfevnnpqvqrqafnelirvvhqllpesslrkrkrsrcggggsggg |
| | | SGGPGPAGMKGLPGSggggsgggsEVQLVESGGGLVQPGNSLRLSCAASGFTFSKFG |
| | | MSWVRQAPGKGLEWVSSISGSGRDTLYAESVKGRFTISRDNAKTTLYLQMNSLR |
| | | PEDTAVYYCTIGGSLSVSSQGTLVTVSSHHHHHH** |
| 434 | ACP240 | EVQLVESGGGLVQPGNSLRLSCAASGFTFSKFGMSWVRQAPGKGLEWVS |
| | | SISGSGRDTLYAESVKGRFTISRDNAKTTLYLQMNSLRPEDTAVYYCTIG |
| | | GSLSVSSQGTLVTVSSggggsggggsggggsiwelkkdvyvveldwypdapgemvvltcdtpee |
| | | dgitwtldqssevlgsgktltiqvkefgdagqytchkggevlshsllllhkkedgiwstdilkdqkepknktflrce |
| | | aknysgrftcwwlttistdltfsvkssrgssdpqgvtcgaatlsaervrgdnkeyeysvecqedsacpaaeeslpie |
| | | vmvdavhklkyenytssffirdiikpdppknlqlkplknsrqvevsweypdtwstphsyfsltfcvqvqgkskr |
| | | ekkdrvftdktsatvicrknasisvraqdryyssswsewasvpcsggggsggggsggggsrvipvsgparclsq |
| | | srnllkttddmvktareklkhysctaedidheditrdqtstlktclplelhknesclatretssttrgsclppqktslmm |
| | | tlclgsiyedlkmyqtefqainaalqnhnhqqiildkgmlvaidelmqslnhngetlrqkppvgeadpyrvkm |
| | | klcillhafstrvvtinrvmgylssaggggsggggsggggsggggsggggsggggsggggsggggsggggsQ |
| | | SVLTQPPSVSGAPGQRVTISCSGSRSNIGSNTVKWYQQLPGTAPKLLIYYN |
| | | DQRPSGVPDRFSGSKSGTSASLAITGLQAEDEADYYCQSYDRYTHPALLF |
| | | GTGTKVTVLggggsggggsggggsQVQLVESGGGVVQPGRSLRLSCAASGFTFS |
| | | SYGMHWVRQAPGKGLEWVAFIRYDGSNKYYADSVKGRFTISRDNSKNT |
| | | LYLQMNSLRAEDTAVYYCKTHGSHDNWGQGTMVTVSSHRHHHH |
| 435 | ACP241 | EAHKSEIAHRYNDLGEQHFKGLVLIAFSQYLQKCSYDEHAKLVQEVTDF |
| | | AKTCVADESAANCDKSLHTLFGDKLCAIPNLRENYGELADCCTKQEPER |
| | | NECFLQHKDDNPSLPPFERPEAEAMCTSFKENPTTFMGHYLHEVARRHPY |
| | | FYAPELLYYAEQYNEILTQCCAEADKESCLTPKLDGVKEKALVSSVRQR |
| | | MKCSSMQKFGERAFKAWAVARLSQTFPNADFAEITKLATDLTKVNKECC |
| | | HGDLLECADDRAELAKYMCENQATISSKLQTCCDKPLLKKAHCLSEVEH |
| | | DT1VIPADLPAIAADFVEDQEVCKNYAEAKDVFLGTFLYEYSRRHPDYSVS |
| | | LLLRLAKKYEATLEKCCAEANPPACYGTVLAEFQPLVEEPKNLVKTNCD |
| | | LYEKLGEYGFQNAILVRYTQKAPQVSTPTLVEAARNLGRVGTKCCTLPE |
| | | DQRLPCVEDYLSAILNRVCLLHEKTPVSEHVTKCCSGSLVERRPCFSALT |
| | | VDETYVPKEFKAETFTFHSDICTLPEKEKQIKKQTALAELVKHKPKATAE |
| | | QLKTVMDDFAQFLDTCCKAADKDTCFSTEGPNLVTRCKDALASGGPGPA |
| | | GMKGLPGSiwelkkdvyvveldwypdapgemvvltcdtpeedgitwtldqssevlgsgktltiqvkefg |
| | | dagqytchkggevlshsllllhkkedgiwstdilkdqkepknktflrceaknysgrftcwwlttistdltfsvkssrg |
| | | ssdpqgvtcgaatlsaervrgdnkeyeysvecqedsacpaaeeslpievmvdavhklkyenytssffirdiikpd |
| | | ppknlqlkplknsrqvevsweypdtwstphsyfsltfcvqvqgkskrekkdrvftdktsatvicrknasisvraq |
| | | dryyssswsewasvpcsggggsggggsggggsrvipvsgparclsqsrnllkttddmvktareklkhysctae |
| | | didheditrdqtstlktclplelhknesclatretssttrgsclppqktslmmtlclgsiyedlkmyqtefqainaalqn |
| | | hnhqqiildkgmlvaidelmqslnhngetlrqkppvgeadpyrvkmklcillhafstrvvtinrvmgylssaS |
| | | GGPGPAGMKGLPGSggggsggggsggggsggggsggggsggggsQSVLTQPPSVSGAP |
| | | GQRVTISCSGSRSNIGSNTVKWYQQLPGTAPKLLIYYNDQRPSGVPDRFS |
| | | GSKSGTSASLAITGLQAEDEADYYCQSYDRYTHPALLFGTGTKVTVLgggg |
| | | sggggsggggsQVQLVESGGGVVQPGRSLRLSCAASGFTFSSYGMHWVRQAP |
| | | GKGLEWVAFIRYDGSNKYYADSVKGRFTISRDNSKNTLYLQMNSLRAED |
| | | TAVYYCKTHGSHDNWGQGTMVTVSSHHHHHH** |
| 436 | ACP242 | iwelkkdvyvveldwypdapgemvvltcdtpeedgitwtldqssevlgsgktltiqvkefgdagqytchkggevlshslll |
| | | lhkkedgiwstdilkdqkepknktflrceaknysgrftcwwlttistdltfsvkssrgssdpqgvtcgaatlsaervrgdnkey |
| | | eysvecqedsacpaaeeslpievmvdavhklkyenytssffirdiikpdppknlqlkplknsrqvevsweypdtwstphs |
| | | yfsltfcvqvqgkskrekkdrvftdktsatvicrknasisvraqdryyssswsewasvpcsggggsggggsggggsrvipv |
| | | sgparclsqsrnllkttddmvktareklkhysctaedidheditrdqtstlktclplelhknesclatretssttrgsclppqktslm |
| | | mtlclgsiyedlkmyqtefqainaalqnhnhqqiildkgmlvaidelmqslnhngetlrqkppvgeadpyrvkmklcillh |
| | | afstrvvtinrvmgylssaSGGPGPAGMKGLPGSggggsggggsggggsggggsggggsggggsQSVLT |
| | | QPPSVSGAPGQRVTISCSGSRSNIGSNTVKWYQQLPGTAPKLLIYYNDQRPSGVP |
| | | DRFSGSKSGTSASLAITGLQAEDEADYYCQSYDRYTHPALLFGTGTKVTVLggggs |
| | | ggggsggggsQVQLVESGGGVVQPGRSLRLSCAASGFTFSSYGMHWVRQAPGKGLE |
| | | WVAFIRYDGSNKYYADSVKGRFTISRDNSKNTLYLQMNSLRAEDTAVYYCKTH |
| | | GSHDNWGQGTMVTVSSSGGPGPAGMKGLPGSEAHKSEIAHRYNDLGEQHFKGL |
| | | VLIAFSQYLQKCSYDEHAKLVQEVTDFAKTCVADESAANCDKSLHTLFGDKLC |
| | | AIPNLRENYGELADCCTKQEPERNECFLQHKDDNPSLPPFERPEAEAMCTSFKEN |
| | | PTTFMGHYLHEVARRHPYFYAPELLYYAEQYNEILTQCCAEADKESCLTPKLDG |
| | | VKEKALVSSVRQRMKCSSMQKFGERAFKAWAVARLSQTFPNADFAEITKLATD |
| | | LTKVNKECCHGDLLECADDRAELAKYMCENQATISSKLQTCCDKPLLKKAHCL |
| | | SEVEHDTMPADLPAIAADFVEDQEVCKNYAEAKDVFLGTFLYEYSRRHPDYSVS |
| | | LLLRLAKKYEATLEKCCAEANPPACYGTVLAEFQPLVEEPKNLVKTNCDLYEKL |
| | | GEYGFQNAILVRYTQKAPQVSTPTLVEAARNLGRVGTKCCTLPEDQRLPCVEDY |
| | | LSAILNRVCLLHEKTPVSEHVTKCCSGSLVERRPCFSALTVDETYVPKEFKAETFT |
| | | FHSDICTLPEKEKQIKKQTALAELVKHKPKATAEQLKTVMDDFAQFLDTCCKAA |
| | | DKDTCFSTEGPNLVTRCKDALAHHHHHH** |
| 437 | ACP243 | vprdcgckpcictvpevssvfifppkpkdvltitltpkvtcvvvdiskddpevqfswfvddvevhtaqtqpreeqfnstfrsv |
| | | selpimhqdwlngkefkcrvnsaafpapiektisktkgrpkapqvytipppkeqmakdkvsltcmitdffpeditvewq |
| | | wngqpaenykntqpimdtdgsyfvysklnvqksnweagntftcsvlheglhnhhtekslshspgkSGGPGPAGM |
| | | KGLPGSiwelkkdvyvveldwypdapgemvvltcdtpeedgitwtldqssevlgsgktltitiqvkefgdagqytchkg |
| | | gevlshsllllhkkedgiwstdilkdqkepknktflrceaknysgrftcwwlttistdltfsvkssrgssdpqgvtcgaatlsaer |
| | | vrgdnkeyeysvecqedsacpaaeeslpievmvdavhklkyenytssffirdiikpdppknlqlkplknsrqvevsweyp |
| | | dtwstphsyfsltfcvqvqgkskrekkdrvftdktsatvicrknasisvraqdryyssswsewasvpcsggggsggggsgg |
| | | ggsrvipvsgparclsqsrnllkttddmvktareklkhysctaedidheditrdqtstlktclplelhknesclatretssttrgscl |
| | | ppqktslmmtlclgsiyedlkmyqtefqainaalqnhnhqqiildkgmlvaidelmqslnhngetlrqkppygeadpyrv |
| | | kmklcillhafstrvvtinrvmgylssaSGGPGPAGMKGLPGSggggsggggsggggsggggsggggsgggg |
| | | sQSVLTQPPSVSGAPGQRVTISCSGSRSNIGSNTVKWYQQLPGTAPKLLIYYNDQ |
| | | RPSGVPDRFSGSKSGTSASLAITGLQAEDEADYYCQSYDRYTHPALLFGTGTKVT |
| | | VLggggsggggsggggsQVQLVESGGGVVQPGRSLRLSCAASGFTFSSYGMHWVRQA |
| | | PGKGLEWVAFIRYDGSNKYYADSVKGRFTISRDNSKNTLYLQMNSLRAEDTAV |
| | | YYCKTHGSHDNWGQGTMVTVSSHHHHHH** |
| 438 | ACP244 | iwelkkdvyvveldwypdapgemvvltcdtpeedgitwtldqssevlgsgktlitiqvkefgdagqytchkggevlshslll |
| | | lhkkedgiwstdilkdqkepknktflrceaknysgrftcwwlttistdltfsvkssrgssdpqgvtcgaatlsaervrgdnkey |
| | | eysvecqedsacpaaeeslpievmvdavhklkyenytssffirdiikpdppknlqlkplknsrqvevsweypdtwstphs |
| | | yfsltcvqvqgkskrekkdrvftdktsatvicrknasisvraqdryyssswsewasvpcsggggsggggsggggsrvipv |
| | | sgparclsqsrnllkttddmvktareklkhysctaedidheditrdqtstlktclplelhknesclatretssttrgsclppqktslm |
| | | mtlclgsiyedlkmyqtefqainaalqnhnhqqiildkgmlvaidelmqslnhngetlrqkppygeadpyrvkmklcillh |
| | | afstrvvtinrvmgylssaSGGPGPAGMKGLPGSggggsggggsggggsggggsggggsggggsQSVLT |
| | | QPPSVSGAPGQRVTISCSGSRSNIGSNTVKWYQQLPGTAPKLLIYYNDQRPSGVP |
| | | DRFSGSKSGTSASLAITGLQAEDEADYYCQSYDRYTHPALLFGTGTKVTVLggggs |
| | | ggggsggggsQVQLVESGGGVVQPGRSLRLSCAASGFTFSSYGMHWVRQAPGKGLE |
| | | WVAFIRYDGSNKYYADSVKGRFTISRDNSKNTLYLQMNSLRAEDTAVYYCKTH |
| | | GSHDNWGQGTMVTVSSSGGPGPAGMKGLPGSvprdcgckpcictypevssvfifppkpkdvltit |
| | | ltpkvtcvvvdiskddpevqfswfvddvevhtaqtqpreeqfnstfrsvselpimhqdwlngkefkcrvnsaafpapiekt |
| | | isktkgrpkapqvytipppkeqmakdkvsltcmitdffpeditvewqwngqpaenykntqpimdtdgsyfyysklnvq |
| | | ksnweagnthcsylheglhnhhtekslshspgkHHHHHH** |
| 439 | ACP245 | EVQLVESGGGLVQPGNSLRLSCAASGFTFSKFGMSWVRQAPGKGLEWVS |
| | | SISGSGRDTLYAESVKGRFTISRDNAKTTLYLQMNSLRPEDTAVYYCTIG |
| | | GSLSVSSQGTLVTVSSSGGPGPAGMKGLPGSiwelkkdvyvveldwypdapgemvvl |
| | | tcdtpeedgitwtldqssevlgsgktltiqvkefgdagqytchkggevlshsllllhkkedgiwstdilkdqkepk |
| | | nktflrceaknysgrftcwwlttistdltfsvkssrgssdpqgvtcgaatlsaervrgdnkeyeysvecqedsacpa |
| | | aeeslpievmvdavhklkyenytssffirdiikpdppknlqlkplknsrqvevsweypdtwstphsyfsltfcvq |
| | | vqgkskrekkdrvftdktsatvicrknasisvraqdryyssswsewasvpcsggggsggggsggggsrvipvs |
| | | gparclsqsrnllkttddmvktareklkhysctaedidheditrdqtstlktclplelhknesclatretssttrgsclpp |
| | | qktslmmtlclgsiyedlkmyqtefqainaalqnhnhqqiildkgmlvaidelmqslnhngetlrqkppvgea |
| | | dpyrvkmklcillhafstrvvtinrvmgylssaSGGPGPAGMKGLPGSggggsggggsggggsggg |
| | | gsggggsggggsQSVLTQPPSVSGAPGQRVTISCSGSRSNIGSNTVKWYQQLPG |
| | | TAPKLLIYYNDQRPSGVPDRFSGSKSGTSASLAITGLQAEDEADYYCQSY |
| | | DRYTHPALLFGTGTKVTVLSGGPGPAGMKGLPGSQVQLVESGGGVVQP |
| | | GRSLRLSCAASGFTFSSYGMHWVRQAPGKGLEWVAFIRYDGSNKYYAD |
| | | SVKGRFTISRDNSKNTLYLQMNSLRAEDTAVYYCKTHGSHDNWGQGTM |
| | | VTVSSHHHHHH |
| 440 | ACP247 | EVQLVESGGGLVQPGNSLRLSCAASGFTFSKFGMSWVRQAPGKGLEWVS |
| | | SISGSGRDTLYAESVKGRFTISRDNAKTTLYLQMNSLRPEDTAVYYCTIG |
| | | GSLSVSSQGTLVTVSSSGGPGPAGMKGLPGSiwelkkdvyvveldwypdapgemvvl |
| | | tcdtpeedgitwtldqssevlgsgktltiqvkefgdagqytchkggevlshsllllhkkedgiwstdilkdqkepk |
| | | nktflrceaknysgrftcwwlttistdltfsvkssrgssdpqgvtcgaatlsaervrgdnkeyeysvecqedsacpa |
| | | aeeslpievmvdavhklkyenytssffirdiikpdppknlqlkplknsrqvevsweypdtwstphsyfsltfcvq |
| | | vqgkskrekkdrvftdktsatvicrknasisvraqdryyssswsewasvpcsggggsggggsggggsrvipvs |
| | | gparclsqsrnllkttddmvktareklkhysctaedidheditrdqtstlktclplelhknesclatretssttrgsclpp |
| | | qktslmmtlclgsiyedlkmyqtefqainaalqnhnhqqiildkgmlvaidelmqslnhngetlrqkppvgea |
| | | dpyrvkmklcillhafstrvvtinrvmgylssaSGGPGPAGMKGLPGSggggsggggsggggsggg |
| | | gsggggsggggsQVQLQESGGGLVQAGGSLRLSCAASGRTFSSVYDMGWFRQ |
| | | APGKDREFVARITESARNTRYADSVRGRFTISRDNAKNTVYLQMNNLEL |
| | | EDAAVYYCAADPQTVVVGTPDYWGQGTQVTVSSHHHHHH |

### INCORPORATION BY REFERENCE

The entire disclosures of all patent and non-patent publications cited herein are each incorporated by reference in their entireties for all purposes.

### OTHER EMBODIMENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

1. A fusion polypeptide comprising at least one of each of:
a) a cytokine polypeptide [A];
b) a cytokine blocking moiety [D]; and
c) a protease-cleavable polypeptide linker [L];
wherein the cytokine polypeptide and the cytokine blocking moiety are operably linked by the protease-cleavable polypeptide linker and the fusion polypeptide has attenuated cytokine receptor activating activity, wherein the cytokine-receptor activating activity of the fusion polypeptide is at least about 10× less than the cytokine receptor activating activity of the polypeptide that contains the cytokine polypeptide that is produced by cleavage of the protease cleavable linker.

2. The fusion polypeptide of claim 1, wherein the cytokine polypeptide is selected from the group consisting of IL-2, IL-7, IL-12, IL-15, IL-18, IL-21, IL-23, TGFβ, IFNα, IFN β, IFNγ, TNF, TGFbeta, CXCL10, CCL19, CCL20, CCL21 and active fragments thereof.

3. The fusion polypeptide of claim 1 or 2, wherein the cytokine polypeptide is a short half-life cytokine polypeptide.

4. The fusion polypeptide of any one of claims 1-3, wherein the each protease-cleavable linker polypeptide independently comprises a sequence that is capable of being cleaved by a protease selected from the group consisting of a kallikrein, thrombin, chymase, carboxypeptidase A, cathepsin G, cathepsin L, an elastase, PR-3, granzyme M, a calpain, a matrix metalloproteinase (MMP), an ADAM, a FAP, a cathepsin L, a plasminogen activator, a cathepsin, a caspase, a tryptase, and a tumor cell surface protease.

5. The fusion polypeptide of claim 8 wherein each protease-cleavable polypeptide independently comprises two or more cleavage sites for the same protease, or two or more cleavage sites that are cleaved by different proteases or at least one of the protease-cleavable polypeptides comprises a cleavage site for two or more different proteases.

6. The fusion polypeptide of any one of claims 1-4, wherein the cytokine blocking moiety is also a half-life extension element.

7. The fusion polypeptide of any one of claims 1-5, wherein the cytokine blocking moiety sterically blocks agonist activity of the cytokine.

8. The fusion polypeptide of claim 5 or 6, wherein the cytokine blocking moiety is human serum albumin, an antigen binding protein, or an antigen binding polypeptide which binds human serum albumin, or a fragment thereof.

9. The fusion polypeptide of any one of claims claim 1-7, wherein the cytokine is free to dissociate from the cytokine blocking moiety after the protease-cleavable polypeptide linker is cleaved by a protease.

10. The fusion polypeptide of any one of claims 1-8, wherein the cytokine blocking moiety inhibits the cytokine polypeptide from activating its cognate receptor.

11. The fusion polypeptide of any of claims 1-9, wherein the fusion polypeptide binds to the cognate receptor of the cytokine polypeptide before cleavage of the protease-cleavable linker.

12. The fusion polypeptide of any one of claims 1-10, further comprising at least one half-life extension element.

13. The fusion polypeptide of any one of claims 1-11, wherein the cytokine blocking moiety non-covalently associates with the cytokine polypeptide, independently of the linker peptide bond.

14. The fusion polypeptide of claim 12, wherein the non-covalent association is pH dependent.

15. The fusion polypeptide of claim 11, wherein the half-life extension element sterically inhibits or blocks activation and/or binding of the cytokine polypeptide to its cognate receptor.

16. The fusion polypeptide of claim 14 wherein the half-life extension element is human serum albumin, a human IgG, a humanized IgG, an scFv, a Fab, an sdAb or a fragment thereof.

17. The fusion polypeptide of claim 9, wherein the cytokine blocking moiety comprises a ligand binding domain, a single domain antibody or scFv that binds the cytokine polypeptide, or an antibody or antibody fragment selected from a single domain antibody and a scFv that binds a receptor of the cytokine polypeptide.

18. The fusion polypeptide of any one of claim 1-16, wherein the cytokine receptor activating activity is determined using a standard in vitro receptor activation assay and equal amounts on a mole basis of the cytokine polypeptide and fusion protein.

19. The fusion polypeptide of any one of claims 10-17, wherein cytokine loses contact with the cytokine blocking moiety and/or half-life extension element after the protease-cleavable sequence is cleaved by a protease.

20. The fusion polypeptide of any one of claims 1-18, wherein the fusion polypeptide comprising at least one of each of [A], [D], and [L], in any of the following orientations:

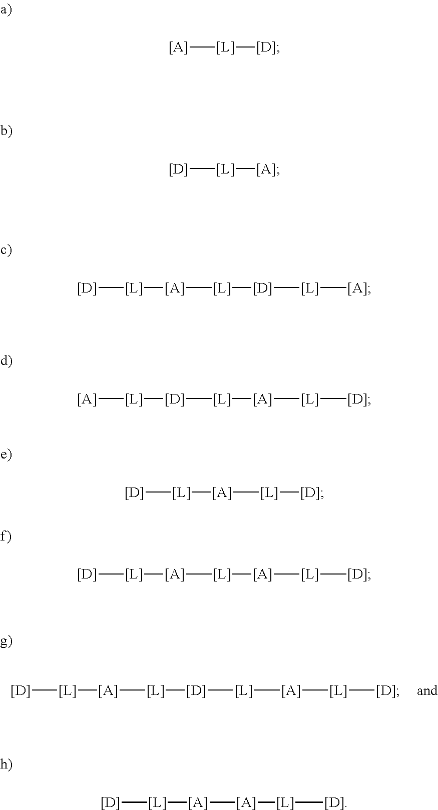

21. The fusion polypeptide of any one of claims 1-19, wherein the fusion polypeptide comprises two cytokine peptides.

22. The fusion polypeptide of claim 1 having the Formula: 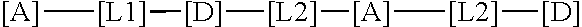
wherein,
A is a cytokine polypeptide;
L1 and L2 are each independently protease-cleavable polypeptide linkers;
D is a cytokine-blocking moiety optionally capable of extending serum half-life; and
wherein L1 is a substrate for a first protease and wherein L2 is a substrate for a second protease.

23. The fusion polypeptide of any one of claims 1-22, wherein the at least one half-life extension element is one half-life extension element, two half-life extension elements, or three half-life extension elements.

24. The fusion polypeptide of any one of claim 1-22, comprising one protease-cleavable linker, two protease-cleavable linkers, three protease-cleavable linkers, four protease-cleavable linkers, five protease-cleavable linkers, six protease-cleavable linkers, or seven protease-cleavable linkers.

25. The fusion polypeptide of any one of claims 1-23 further comprising a tumor specific antigen binding peptide.

26. The fusion polypeptide of claim 25 where the tumor specific antigen binding peptide is operably linked to the cytokine polypeptide by a non-cleavable linker.

27. The fusion polypeptide of claim 25 where the tumor specific antigen binding peptide is operably linked to the cytokine polypeptide by a cleavable linker.

28. The fusion polypeptide of any one of claims 1-26, wherein the cytokine polypeptide has a serum half-life comparable to the corresponding naturally occurring cytokine.

29. The fusion polypeptide of any one of claims 1-26, wherein the serum half-life of the polypeptide that contains the cytokine polypeptide that is produced by cleavage of the fusion protein is comparable to the corresponding naturally occurring cytokine.

30. The fusion polypeptide of any one of claims 1-26, wherein the cytokine polypeptide is a mutein that has a short serum half-life and is rapidly cleared following cleavage of the fusion polypeptide.

31. A fusion polypeptide comprising at least one of each of:
a) An interferon polypeptide [A];
b) An interferon blocking moiety [D]; and
c) a protease-cleavable polypeptide linker [L]; and
wherein the interferon polypeptide and the interferon blocking moiety are operably linked by the protease-cleavable polypeptide linker the fusion polypeptide has attenuated interferon-receptor activating activity, wherein the interferon-receptor activating activity of the fusion polypeptide is at least about 10× less than the interferon-receptor activating activity of the interferon polypeptide that is produced by cleavage of the protease cleavable linker.

32. The fusion polypeptide of claim 31 having the Formula: 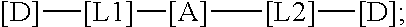
wherein,
A is an interferon alpha (IFN(a) polypeptide;
L1 and L2 are each independently are a protease-cleavable polypeptide linker;
D is an IFNα blocking moiety which also extends in vivo half-life; and
wherein the interferon alpha polypeptide and the interferon alpha blocking moiety are operably linked by the protease-cleavable polypeptide linker the fusion polypeptide has attenuated interferon alpha-receptor activating activity, wherein the interferon alpha-receptor activating activity of the fusion polypeptide is at least about 10× less than the interferon-receptor activating activity of the interferon alpha polypeptide that is produced by cleavage of the protease cleavable linker, and wherein when cleavage of both linkers occurs, in vivo half-life of the interferon alpha polypeptide is substantially similar to that of naturally occurring interferon alpha.

33. The fusion polypeptide of claim 31 having the Formula: 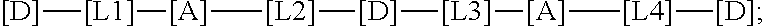
wherein,
A is an interferon gamma (IFNγ) polypeptide;
L1, L2, L3, and L4 are each independently a protease-cleavable polypeptide linkers;
D is an IFNγ blocking moiety capable of extending serum half-life; and
wherein the interferon gamma polypeptide and the interferon gamma blocking moiety are operably linked by the protease-cleavable polypeptide linker the fusion polypeptide has attenuated interferon gamma-receptor activating activity, wherein the interferon gamma-receptor activating activity of the fusion polypeptide is at least about 10× less than the interferon gamma-receptor activating activity of the interferon gamma polypeptide that is produced by cleavage of the protease cleavable linker, and wherein when cleavage of both linkers occurs, in vivo half-life of the interferon gamma polypeptide is substantially similar to that of naturally occurring interferon gamma.

34. The fusion polypeptide of any one of claims 31-33, wherein the protease-cleavable linker polypeptide comprises a sequence that is capable of being cleaved by a protease selected from the group consisting of a kallikrein, thrombin, chymase, carboxypeptidase A, cathepsin G, cathepsin L, an elastase, PR-3, granzyme M, a calpain, a matrix metalloproteinase (MMP), an ADAM, a FAP, a cathepsin L, a plasminogen activator, a cathepsin, a caspase, a tryptase, and a tumor cell surface protease.

35. The fusion polypeptide of claim 34 wherein each protease-cleavable polypeptide independently comprises two or more cleavage sites for the same protease, or two or more cleavage sites that are cleaved by different proteases or at least one of the protease-cleavable polypeptides comprises a cleavage site for two or more different proteases.

36. The fusion polypeptide of claim 31, wherein the interferon is interferon alpha or interferon gamma.

37. The fusion polypeptide of claim 31 having the Formula: 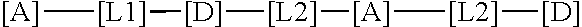
wherein,
A is an interferon polypeptide;
L1 and L2 are each independently protease-cleavable polypeptide linkers;
D is an interferon-blocking moiety optionally capable of extending serum half-life; and
wherein L1 is a substrate for a first protease and wherein L2 is a substrate for a second protease.

38. The fusion polypeptide of any one of claims 31-37, wherein the interferon blocking moiety is also a half-life extension element.

39. The fusion polypeptide of any one of claims 31-38, wherein the interferon blocking moiety sterically blocks agonist activity of the cytokine.

40. The fusion polypeptide of claim 39, wherein the interferon blocking moiety is human serum albumin, or an antigen binding polypeptide which binds human serum albumin.

41. The fusion polypeptide of any one of claims 31-0, wherein the interferon is free to dissociate from the interferon blocking moiety after the protease-cleavable polypeptide linker is cleaved by a protease.

42. The fusion polypeptide of any of claims 31-41, wherein the interferon blocking moiety inhibits the interferon polypeptide from activating its cognate receptor.

43. The fusion polypeptide of any of claims 31-42, wherein the fusion polypeptide binds to the cognate receptor for the interferon polypeptide before cleavage of the protease cleavable linker.

44. The fusion polypeptide of any one of claims 31-43, further comprising at least one half-life extension element.

45. The fusion polypeptide of any one of claim 44, wherein the interferon blocking moiety non-covalently associates with the interferon polypeptide.

46. The fusion polypeptide of any one of claims 31-45, wherein the noncovalent association is pH dependent.

47. The fusion polypeptide of claim-46, wherein the half-life extension element sterically inhibits or blocks activation and/or binding of the interferon polypeptide to its cognate receptor.

48. The fusion polypeptide of claim 44 wherein the half-life extension element is human serum albumin, a human IgG, or a fragment thereof.

49. The fusion polypeptide of claims 44-48, wherein the half-life extension element does not bind the interferon polypeptide.

50. The fusion polypeptide of claim 31, wherein the interferon blocking moiety comprises a ligand binding domain or fragment of a cognate receptor for the interferon, a single domain antibody or scFv that binds the interferon polypeptide, or an antibody or antibody fragment (e.g., single domain antibody, scFv) that binds a receptor of the interferon.

51. The fusion polypeptide of any one of claims 31-50, wherein the cognate interferon receptor activation activity is determined using a standard in vitro receptor activation assay and equal amounts on a mole basis of the fusion polypeptide and interferon polypeptide.

52. The fusion polypeptide of any one of claims 31-51, wherein interferon is free to dissociate from with the interferon blocking moiety and/or half-life extension element after the protease-cleavable sequence is cleaved by a protease.

53. The fusion polypeptide of any one of claims 31-52, wherein the fusion polypeptide comprises at least one of each of [A], [B], and [L], in any of the following orientations:

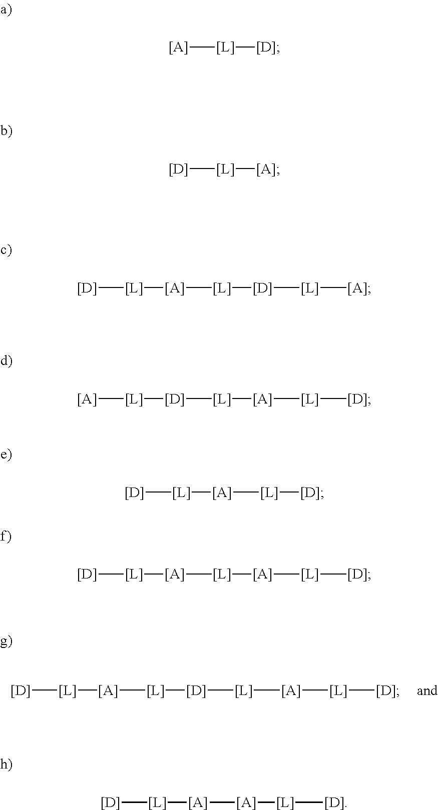

54. The fusion polypeptide of any one of claims 31-53 wherein is the fusion polypeptide comprises two cytokine peptides.

55. The fusion polypeptide of claim 54, wherein the two cytokine polypeptides are two different cytokine peptides.

56. The fusion polypeptide of any one of claims 44-55, wherein the at least one half-life extension element is one half-life extension element or two half-life extension elements.

57. The fusion polypeptide of any one of claims 31-56, comprising one protease-cleavable polypeptide linker is one protease-cleavable linker, two protease-cleavable linkers, three protease-cleavable linkers, four protease-cleavable linkers, five protease-cleavable linkers, six protease-cleavable linkers, or seven protease-cleavable linkers.

58. The fusion polypeptide of any one of claims 31-57, further comprising a tumor specific antigen binding peptide.

59. The fusion polypeptide of claim 58, where the tumor specific antigen binding peptide is operably linked to the interferon polypeptide by a non-cleavable linker.

60. The fusion polypeptide of claim 58, where the tumor specific antigen binding peptide is operably linked to the interferon polypeptide by a cleavable linker.

61. A nucleic acid encoding the fusion polypeptide of any one of claims 1-60.

62. A vector comprising the nucleic acid of claim 61.

63. A host cell comprising the vector of claim 62 or nucleic acid of claim 61.

64. A method of making a pharmaceutical composition, comprising culturing the host cell of claim 63 under suitable conditions for expression and collection of desired polypeptides.

65. A pharmaceutical composition comprising i) a fusion polypeptide of any one of claims 1-60, and ii) a pharmaceutically acceptable excipient.

66. A method for treating a tumor, comprising administering to a subject in need thereof an effective amount of a fusion polypeptide of any one of claims 1-60.

67. A fusion polypeptide of any one of claims 1-60 for use as a medicament.

68. A fusion polypeptide of any one of claims 1-60 for use in treating a tumor in a subject in need thereof.

69. A pharmaceutical composition for treating a tumor in a subject in need thereof comprising as an active ingredient a fusion polypeptide of any one of claims 1-60.

70. The fusion polypeptide of claim 1, wherein the fusion protein is selected from the group consisting of SEQ ID NOs.: 193-271 or ACP200-208, ACP211, ACP213-ACP215, ACP240-245, ACP247, ACP284-ACP292, ACP296, ACP297, ACP298-ACP300, ACP302-ACP306, ACP309-ACP314, ACP336-ACP359, ACP371-ACP379, ACP383-ACP38-ACP634, ACP439-ACP447, ACP451-ACP471, or any combination thereof.

71. A nucleic acid encoding the fusion polypeptide of claim 70.

72. A pharmaceutical composition for treating a tumor in a subject in need thereof comprising an active ingredient of a fusion polypeptide of claim 70.
